(19) United States
(12) Patent Application Publication
(10) Pub. No.: US 2025/0122171 A1
(43) Pub. Date: Apr. 17, 2025

(54) Substituted Amine Compounds, Compositions and Methods of Use

(71) Applicant: Deep Apple Therapeutics, Inc., San Francisco, CA (US)

(72) Inventors: Stefan Gahbauer, San Francisco, CA (US); Jason R. Zbieg, Montara, CA (US); Robert L. Dow, Groton, CT (US); Liang Zhao, Foster City, CA (US); Craig Stivala, San Mateo, CA (US)

(21) Appl. No.: 18/888,376

(22) Filed: Sep. 18, 2024

Related U.S. Application Data
(60) Provisional application No. 63/678,839, filed on Aug. 2, 2024.
(60) Provisional application No. 63/640,903, filed on May 1, 2024.
(60) Provisional application No. 63/584,045, filed on Sep. 20, 2023.

(51) Int. Cl. C07D 401/14; A61K 31/454; A61K 31/4545; A61K 31/497; A61K 31/5377; C07D 233/88; C07D 413/14; C07D 417/14; C07D 487/04; C07D 513/04

(52) U.S. Cl. CPC C07D 401/14; A61K 31/454; A61K 31/4545; A61K 31/497; A61K 31/5377; C07D 233/88; C07D 413/14; C07D 417/14; C07D 487/04; C07D 513/04

(57) ABSTRACT

The present disclosure provides compounds that are useful for the treatment of conditions mediated by MRGPRX2. Also provided are pharmaceutical compositions containing such compounds, and methods of treatment using such compounds.

### FIELD OF INVENTION

The present disclosure provides compounds that modulate MRGPRX2 and are therefore useful for the treatment of conditions, diseases and/or disorders mediated by MRGPRX2. Also provided are pharmaceutical compositions containing such compounds and processes for preparing such.

### BACKGROUND

Mas-related G-protein coupled receptor member X2 is a protein that, in humans, is encoded by the MRGPRX2 gene and is most abundant on cutaneous mast cells.

Agonists are gyrase inhibitors like ciprofloxacin and non-depolarizing neuromuscular blocking agents like atracurium as well as vancomycin. Activation of MRGPRX2 leads to mast cell degranulation with subsequent pseudo-allergic reactions.

Mature mammalian mast cells ordinarily reside: Near blood vessels or nerves; beneath or within epithelia; within airways, gastrointestinal, and genitourinary tracts; and near smooth muscle and mucus-producing glands. Classically, mast cells are activated by IgE antibodies, secreting a wide range of substances with local and systemic effects including histamine, serotonin, proteases, chemokines, and cytokines. Indeed, mast cells are implicated in the progression and/or maintenance of many diseases (Nature Immunology, 6, 135-142 (2005)).

Recent work has emphasized the role of the Mas-related G protein-coupled receptor (MRGPR) family, specifically, Mrgprb2, in mast cell activation (Nature, 519, 237-241 (2015)). Mrgprb2 is the mouse receptor for several cationic molecules, collectively called basic secretagogues, and the ortholog of the human receptor MRGPRX2 (Adv. Immunol., 136, 123-62 (2017)). To date, Mrgprb2 and MRGPRX2 have been reported to be expressed only on certain populations of mast cells (Nature Immunology, 25 17, 878-887 (2016); Annu. Rev. Immunol., 38, 49-77 (2020)). This knowledge provides the opportunity to target mast cell degranulation in a very precise manner.

WO 2022/073904 discloses MrgX2 amide antagonists useful for treating MrgX2 mediated diseases such as urticaria and atopic dermatitis. WO2022/152852, WO2022/152853 and WO2022/073905 also disclose MrgX2 amide antagonists useful for treating MrgX2 mediated diseases. WO2021/092262 discloses MRGPRX2 antagonists, typically amides, useful for the treatment of inflammatory conditions such as atopic dermatitis.

In summary, a potent, selective antagonist of MRGPRX2 that blocks IgE-independent mast cell de-granulation is expected to provide therapeutic benefit in mast-cell driven pathologies including skin disorders such as urticaria, atopic dermatitis and rosacea as well as additional indications like inflammatory disease conditions and autoimmune disease conditions. Thus, there is a need and a continuing search in this field of art for new therapies.

### SUMMARY

The present invention relates to compounds of Formula (I), as described herein including stereochemical isomeric forms thereof and pharmaceutically acceptable salts thereof, which may be useful for mediating MRGPRX2 and/or treating or preventing MRGPRX2 mediated conditions and diseases. More specifically, in some embodiments, provided herein are compounds of Formula (I) or a pharmaceutically acceptable salt thereof:

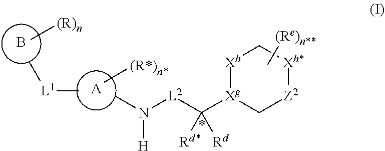

wherein the “*” at the carbon to which R^{d} and R^{d*} are bonded is a first stereo center when R^{d} and R^{d*} are different;
Z² is CH₂ or

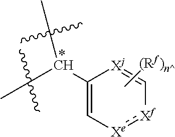

wherein the wavy lines represent the points of attachment of the Z² group to the X^{h*} and the —CH₂ of the 6-membered ring;
wherein the “*” at the carbon atom in Z² designates a second stereocenter;
with the proviso that when Z² is —CH₂, then Ring A is not a thiazolyl;
Ring B is a 6-membered aryl or 5- or 6-membered heteroaryl group;
each R is independently selected from hydrogen, CN, SF₅, halo, (C₁-C₆)alkyl, —(C₀-C₆)alkyl-NH₂, —(C₀-C₆)alkyl-NH((C₁-C₆)alkyl), —(C₀-C₆)alkyl-N((C₁-C₆)alkyl)((C₁-C₆)alkyl), —(C₀-C₆)alkyl-(C₃-C₈)cycloalkyl, —(C₀-C₆)alkyl-aryl, 5- or 6-membered heteroaryl(C₁-C₄)alkyl-, (C₂-C₆)alkenyl, —(C₂-C₆)alkenyl-(C₃-C₈)cycloalkyl, —OH, (C₁-C₄)alkoxy, —O—(C₁-C₆)alkyl-(C₃-C₈)cycloalkyl, —O—(C₁-C₆)alkyl-aryl, —O—(C₁-C₆)alkyl-5-6 membered heteroaryl, —O—(C₂-C₆)alkenyl, —O—(C₃-C₈)cycloalkyl, —O-aryl, —O-heteroaryl, —C(O)-aryl, —CO₂H, —CO₂(C₁-C₆)alkyl, —CO₂(C₃-C₈)cycloalkyl, —O₂C(C₁-C₆)alkyl, —O₂C(C₃-C₈)cycloalkyl, or 5-6 membered heteroaryl, wherein any said (C₁-C₆)alkyl, —(C₀-C₆)alkyl-(C₃-C₈)cycloalkyl, —(C₀-C₆)alkyl-aryl, (C₁-C₄)alkoxy, —O—(C₁-C₆)alkyl-(C₃-C₈)cycloalkyl, —O—(C₃-C₈)cycloalkyl, —O-aryl, 5-6 membered heteroaryl, —C(O)-aryl, or 5-6 membered heteroaryl is optionally substituted one, two, or three times by R¹ wherein each R¹ is independently halo, —CN, —SF₅, —OH, (C₁-C₆)alkyl, (C₁-C₄)alkoxy, (C₃-C₈)cycloalkyl, —(C₁-C₆)—OH, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), —O—(C₃-C₈)cycloalkyl, -aryl, 5-6 membered heteroaryl, wherein (C₁-C₆)alkyl, (C₃-C₈)cycloalkyl, aryl, or 5-6 membered heteroaryl is further optionally substituted by one, two, or three substituents independently selected from halo, (C₁-C₆)alkyl, —(C₁-C₆)alkyl-OH, —(C₁-C₆)alkyl-O—(C₁-C₆)alkyl, or (C₁-C₄)alkoxy;
n is 0 or an integer from 1 to 5;
L¹ is C(R^{c})(R^{c*}), O, SO₂ or C(O);
each of R^{c} and R^{c*} is independently H, D, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halo, (C₁-C₆)alkyl, halo(C₁-C₆)alkyl, and (C₃-C₈)cycloalkyl;
Ring A is a 5-membered arylene or a 5-membered heteroarylene group;
each R* is independently selected from hydrogen, OH, halo, (C₁-C₆)alkyl, halo(C₁-C₆)alkyl, (C₂-C₆)alkenyl, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), (C₂-C₆)alkenyl, —O—(C₁-C₆)alkyl, —NH₂, —NH((C₁-C₆)alkyl), —N((C₁-C₆)alkyl)((C₁-C₆)alkyl), —NCH₂, or —CHNH; or two R* groups taken together with the atoms to which they are attached, form a 5-, 6-, or 7-membered ring optionally containing one, two, or three heteroatoms independently selected from oxygen, nitrogen, and sulfur, wherein said ring is optionally substituted by one or two substituents independently selected from halo, (C₁-C₆)alkyl, —(C₁-C₆)alkyl-(C₃-C₈)cycloalkyl, halo(C₁-C₆)alkyl, and (C₃-C₈)cycloalkyl;
n* is 0 or an integer from 1 to 3;
or one of R^{c} or R^{c*} and R* taken together with the atoms to which they are bonded form a 3- to 6-membered ring fused to Ring A;
L² is —C(O)— or

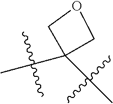 [SMILES: *C1(*)COC1]

wherein the wavy line represents the points of attachment to the NH and the C(R^{d})(R^{d*}) groups;
R^{d} and R^{d*} are each independently H or (C₁-C₃)alkyl, or (C₁-C₃)alkoxy, or R^{d} and R^{d*} cyclize to form a 3 to 6 membered cycloalkyl or heterocycloalkyl ring;
X^{h} and X^{h*} are each independently C(R^{e})₂, N(R^{e}), or O;
X^{g} is CH or N;
each R^{e} is independently H, halo, (C₁-C₃)alkyl, halo(C₁-C₃)alkyl, or (C₁-C₃)alkoxy, or any two R^{e} groups, on the same ring atom or on adjacent ring atoms, cyclize to form a 3 to 6 membered fused or spiro ring;
n** is 0 or an integer from 1 to 8;
X^{j} is N, NR^{f}, C(R^{f})₂, C(R^{f}) or CH;
X^{e} is N, NR^{f}, C(R^{f})₂ or C(R^{f});
X^{f} is SO₂, N, C-(halo(C₁-C₃)alkyl), C—(C₁-C₃)alkyl, C═O or N⁺—O—;
[symbol] represents a double or single bond as needed to satisfy atom valences;
each R^{f} is independently H or halo, (C₁-C₆)alkyl, (C₃-C₈)cycloalkyl, (C₁-C₄)alkoxy, —(C₁-C₆)alkyl-OH, —(C₁-C₆)alkyl-O—(C₁-C₆)alkyl, —(C₁-C₆)alkyl-NH₂, —(C₁-C₆)alkyl-NH((C₁-C₆)alkyl), —(C₁-C₆)alkyl-N—((C₁-C₆)alkyl)((C₁-C₆)alkyl), —(C₁-C₆)alkyl-NH((C₁-C₆)alkyl)-OH, —(C₁-C₆)alkyl-NH((C₁-C₆)alkyl)-O—(C₁-C₆)alkyl), —(C₁-C₆)alkyl-N—((C₁-C₆)alkyl)((C₁-C₆)alkyl)-OH, —(C₁-C₆)alkyl-N—((C₁-C₆)alkyl)((C₁-C₆)alkyl)-O—(C₁-C₆)alkyl), —(C₁-C₆)alkyl-NH—(C₃-C₈)cycloalkyl, —(C₁-C₆)alkyl-NH-4- to 6-membered heterocycloalkyl, —(C₁-C₆)alkyl-4- to 6-membered heterocycloalkyl, —(C₁-C₆)alkyl-NHC(O)—((C₁-C₆)alkyl), —(C₁-C₆)alkyl-SO₂-(C₁-C₆)alkyl, —(C₁-C₆)alkyl-SO₂—NH—(C₁-C₆)alkyl, —CN, —C(O)NH₂, —C(O)NH(C₁-C₆)alkyl), —C(O)N(C₁-C₆)alkyl) (C₁-C₆)alkyl), —NH₂, —NH(C₁-C₆)alkyl, —N((C₁-C₆)alkyl)((C₁-C₆)alkyl), —SO₂((C₁-C₆)alkyl), —SO₂—NH((C₁-C₆)alkyl), or aryl, wherein any said (C₁-C₆)alkyl, (C₃-C₈)cycloalkyl, —(C₁-C₆)alkyl-OH, —(C₁-C₆)alkyl-NH₂, —(C₁-C₆)alkyl-NH((C₁-C₆)alkyl), —(C₁-C₆)alkyl-NH—(C₃-C₈)cycloalkyl, —(C₁-C₆)alkyl-NH-4- to 6-membered heterocycloalkyl, or —(C₁-C₆)alkyl-4- to 6-membered heterocycloalkyl is optionally substituted one, two, or three times by halo, OH, or any two R^{f} groups, on the same ring atom or on adjacent ring atoms, cyclize to form a 3 to 6 membered fused or spiro ring;
n\hat{ } is 0 or an integer from 1 to 4.

Also provided herein, in some embodiments, are pharmaceutical compositions comprising a compound of Formula (I) or a pharmaceutically acceptable salt thereof and a pharmaceutically acceptable excipient.

Further provided herein, in some embodiments, are methods of modulating MRGPRX2 activity; and/or methods of modulating mast cell degranulation, and/or methods of treating, preventing or ameliorating an MRGPRX2-mediated disease or disorder in a subject in need thereof, wherein each of these methods independently comprises administering an effective amount of a compound of Formula (I) or a pharmaceutically acceptable salt thereof, or administering an effective amount of a pharmaceutical composition comprising a compound of Formula (I) or a pharmaceutically acceptable salt thereof and a pharmaceutically acceptable excipient.

Further provided herein, in some embodiments, are methods for treating, preventing or ameliorating a disease or conditions chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain, preferably wherein the condition is atopic dermatitis.

Also provided herein are compounds of Formula (I) or a pharmaceutically acceptable salt thereof for use as a medicament. In some embodiment the compound is for use in treating disease or disorder mediated by MRGPRX2.

Also provided herein is the use of a compound of Formula (I) or a pharmaceutically acceptable salt thereof in the manufacture of a medicament for treating a MRGPRX2-mediated disease or disorder.

### BRIEF DESCRIPTION OF THE DRAWINGS

FIG. 1A shows the experimental X-ray powder diffraction (XRPD) pattern of a crystalline sample of Compound 489.

FIG. 1B is a table of the characteristic peaks in the XRPD pattern of Compound 489 in FIG. 1A. Angle [2Th] refers to the two-theta degree position on the x-axis for certain peaks and Rel Intensity [%] refers to the relative intensity shown on the y-axis of the peak, and d Value [Å] refers to the interplanar spacing in angstroms.

FIG. 2A shows the asymmetric unit of Compound 489 as determined by micro electronic diffraction (MicroED) with hydrogen bonds shown as dashed lines.

FIG. 2B shows the results of MicroED structure determination.

### DETAILED DESCRIPTION

Unless otherwise stated, the following terms used in the specification and claims are defined for the purposes of this disclosure and have the following meanings.

“Alkyl” means a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to, methyl, ethyl, n-propyl, isopropyl, n-butyl, isobutyl, s-butyl, t-butyl, pentyl, and hexyl. and the like.

“Alkenyl” means a saturated, straight or branched hydrocarbon moiety having the specified number of carbon atoms and at least one carbon-carbon double bond. The term “(C₁-C₆)alkenyl” refers to an alkenyl moiety containing from 1 to 6 carbon atoms. Exemplary alkenyls include, but are not limited to, ethenyl, n-propenyl, isopropenyl, n-butenyl, isobutenyl, s-butenyl, t-butenyl, pentenyl, and hexenyl. and the like.

“Alkoxy” means a —OR radical where R is alkyl as defined above, e.g., methoxy, ethoxy, propoxy, or 2-propoxy, n-, iso-, or tert-butoxy, and the like. The term “(C₁-C₄)alkoxy” refers to a straight- or branched-chain hydrocarbon radical having at least 1 and up to 4 carbon atoms attached through an oxygen linking atom.

When the term “alkyl” is used in combination with other substituent groups, such as “halo(C₁-C₄)alkyl”, “aryl(C₁-C₄)alkyl-”, or “(C₁-C₄)alkoxy(C₁-C₄)alkyl-”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, —CF₃ (trifluoromethyl), —CCl₃ (trichloromethyl), 1,1-difluoroethyl, 2-fluoro-2-methylpropyl, 2,2-difluoropropyl, 2,2,2-trifluoroethyl, and hexafluoroisopropyl. Examples of “aryl(C₁-C₄)alkyl” or “phenyl(C₁-C₄)alkyl” groups useful in the present invention include, but are not limited to, benzyl and phenethyl. Examples of “(C₁-C₄)alkoxy(C₁-C₄)alkyl-” groups useful in the present invention include, but are not limited to, methoxymethyl, methoxyethyl, methoxyisopropyl, ethoxymethyl, ethoxyethyl, ethoxyisopropyl, isopropoxymethyl, isopropoxyethyl, isopropoxyisopropyl, t-butoxymethyl, t-butoxy ethyl, and t-butoxyisopropyl.

As used herein, the term “cycloalkyl” refers to a non-aromatic, saturated, cyclic hydrocarbon ring containing the specified number of carbon atoms. The term (C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

As used herein, “heterocycloalkyl” represents a group or moiety comprising a nonaromatic, monovalent monocyclic radical, which is saturated or partially unsaturated and includes one or two heteroatoms selected independently from oxygen, sulfur, and nitrogen. The term “4- to 6-membered heterocycloalkyl” refers to a heterocycloalkyl group containing 4, 5, or 6 ring atoms, which includes one or two heteroatoms selected independently from oxygen, sulfur, and nitrogen. Illustrative examples of 4- to 6-membered heterocycloalkyl groups useful in the present invention include, but are not limited to azetidinyl, oxetanyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiol any 1, 1,3-oxathianyl, 1,3-dithianyl, 1,4-oxathiolanyl, 1,4-oxathianyl, and 1,4-dithianyl. As used herein, “amino” refers to primary, secondary and tertiary nitrogen containing groups.

“Halo” means fluoro, chloro, bromo, or iodo; in one embodiment fluoro or chloro.

As used herein, “aryl” refers to monovalent optionally substituted monocyclic, fused bicyclic, or fused tricyclic groups having 6 to 16 carbon atoms and having at least one aromatic ring that complies with Huckel's Rule. Examples of “aryl” groups are phenyl, naphthyl, indenyl, dihydroindenyl, anthracenyl, phenanthrenyl, and the like. This term also encompasses bicyclic and tricyclic aryl compounds containing an aryl ring moiety fused to a cycloalkyl ring moiety, containing 5 to 16 ring atoms. As used herein, “arylene” refers to a bivalent aryl group.

“Heteroaryl” represents a group or moiety comprising an aromatic monovalent monocyclic or bicyclic radical, or tricyclic radical containing 5 to 16 ring atoms, including 1 to 6 heteroatoms independently selected from nitrogen, oxygen and sulfur. This term also encompasses bicyclic and tricyclic heterocyclic-aryl compounds containing an aryl ring moiety fused to a heterocycloalkyl ring moiety, containing 5 to 16 ring atoms, including 1 to 4 heteroatoms independently selected from nitrogen, oxygen and sulfur. Illustrative examples of heteroaryl groups useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl. Examples of 10- to 16-membered tricyclic heteroaryl group include [1,3]dioxolo[4′,5′:4,5]benzo[1,2-d]thiazole groups, and the like.

As used herein, “5- or 6-membered heteroaryl” represents a group or moiety comprising an aromatic monovalent monocyclic radical, containing 5 or 6 ring atoms, including at least one carbon atom and 1 to 4 heteroatoms independently selected from nitrogen, oxygen, and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, pyrimidinyl, and triazinyl. As used herein, “heteroarylene” refers to a bivalent heteroaryl group.

“Oxo” means an ═(O) group where the oxygen is bound to any atom and “carbonyl” means a >C(O) or >C═O or C═O group.

“Mammal” as used herein means domesticated animals (such as dogs, cats, and horses), and humans. In one embodiment, mammal is a human.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. It is understood that the pharmaceutically acceptable salts are non-toxic. For a review on suitable salts, see Handbook of Pharmaceutical Salts. Properties, Selection, and Use by Stahl and Wermuth (Wiley-VCH, 2002). Additional information on suitable pharmaceutically acceptable salts can be found in Remington The Science and Practice of Pharmacy, 23rd ed., Elsevier Science, 2020, which is incorporated herein by reference.

“Optional” or “optionally” means that the subsequently described event or circumstance may but need not occur, and that the description includes instances where the event or circumstance occurs and instances in which it does not. For example, “heterocycloalkyl group optionally substituted with an alkyl group” means that the alkyl may but need not be present, and the description includes situations where the heterocycloalkyl group is substituted with an alkyl group and situations where the heterocycloalkyl group is not substituted with alkyl.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

A “therapeutically effective amount” means the amount of a compound of Formula (I) (or any of the embodiments thereof described herein), that, when administered to a mammal for treating a disease, is sufficient to treat the disease. The “therapeutically effective amount” will vary depending on the compound, the disease and its severity and the age, weight, etc., of the mammal to be treated.

The compounds described herein may in some cases exist as diastereomers, enantiomers, or other stereoisomeric forms. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis. Those skilled in the art are familiar with methods for determining absolute stereochemistry e.g., X-Ray powder diffraction. Stereoisomers may also be obtained by stereoselective synthesis into their compounding pure enantiomers by forming a diastereomeric salt with an optically pure chiral base or acid (e.g., 1-phenyl-ethylamine or tartaric acid) and separating the diastereomers by fractional crystallization followed by neutralization to break the salt, thus providing the corresponding pure enantiomers.

Certain compounds of Formula (I) (or any of the embodiments thereof described herein) and/or a pharmaceutically acceptable salt thereof may exist as tautomers and/or geometric isomers. All possible tautomers and cis and trans isomers, as individual forms and mixtures thereof, are within the scope of this disclosure. For example, pyrazole tautomers as shown below are equivalent structures. The depiction of one such structure is intended to encompass both structures.

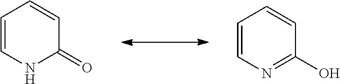 [SMILES: O=c1cccc[nH]1 ; Oc1ccccn1]

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

The compounds described herein include hydrates and solvates of the compounds or pharmaceutically acceptable salts thereof. The term solvate is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The term hydrate is employed when said solvent is water. Pharmaceutically acceptable solvates include hydrates and other solvates wherein the solvent of crystallization may be isotopically substituted, e.g., D₂O. d-acetone, d-DMSO. The solvates and/or hydrates preferably exist in crystalline form. A classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

The present disclosure also includes the prodrugs of compounds of Formula (I) (or any of the embodiments thereof described herein) and/or a pharmaceutically acceptable salt thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt thereof are also within the scope of this disclosure.

Also included within the scope of the invention are multi-component complexes (other than salts and solvates) wherein the drug and at least one other component are present in stoichiometric or non-stoichiometric amounts. The compounds of the invention may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins.

The present disclosure also includes polymorphic forms (amorphous as well as crystalline).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast colling. The presence of polymorphs may be determined by solid probe NMR spectroscopy, IR spectroscopy, differential scanning calorimetry, powder X-ray diffraction or such other techniques.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure. For example, compounds of the present invention wherein L¹ is methylene and a hydrogen is replaced by deuterium is envisioned.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Also included within the scope of the invention are metabolites of compounds of Formula I, that is, compounds formed in vivo upon administration of the drug. Some examples of metabolites in accordance with the invention include
(i) where the compound of Formula I contains a methyl group, an hydroxymethyl derivative thereof (—CH₃—>—CH₂OH):
(ii) where the compound of Formula I contains an alkoxy group, an hydroxy derivative thereof (—OR—>—OH);
(iii) where the compound of Formula I contains a tertiary amino group, a secondary amino derivative thereof (—NRR—>—NHR or —NHR);
(iv) where the compound of Formula I contains a secondary amino group, a primary derivative thereof (—NHR—>—NH₂);
(v) where the compound of Formula I contains a phenyl moiety, a phenol derivative thereof (-Ph->-PhOH); and
(vi) where the compound of Formula I contains an amide group, a carboxylic acid derivative thereof (—CONH₂—>COOH).

In one aspect, provided here is a compound of Formula (I) or a pharmaceutically acceptable salt thereof:

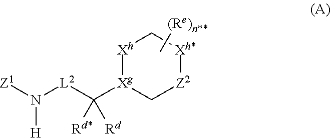

wherein Z¹ is a 10- to 16-membered tricyclic aryl or heteroaryl group substituted with 0, 1, 2 or 3 R groups, or

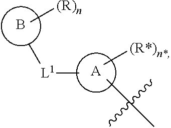

wherein the wavy line represents the point of attachment of the Z¹ group to the NH group;
Z² is CH₂ or

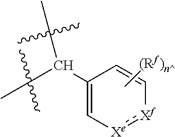

wherein the wavy lines represents the points of attachment of the Z² group to the 6-membered ring;
Ring B is a 5- or 6-membered aryl or heteroaryl group;
each R is independently selected from hydrogen, CN, SF₅, halogen, (C₁-C₆)alkyl, —(C₀-C₆)alkyl-NH₂, —(C₀-C₆)alkyl-NH((C₁-C₆)alkyl), —(C₀-C₆)alkyl-N((C₁-C₆)alkyl)((C₁-C₆)alkyl), —(C₀-C₆)alkyl-(C₃-C₈)cycloalkyl, —(C₀-C₆)alkyl-aryl, 5- or 6-membered heteroaryl(C₁-C₄)alkyl-, (C₂-C₆)alkenyl, —(C₂-C₆)alkenyl-(C₃-C₈)cycloalkyl, —OH, (C₁-C₄)alkoxy, —O—(C₁-C₆)alkyl-(C₃-C₈)cycloalkyl, —O—(C₁-C₆)alkyl-aryl, —O—(C₁-C₆)alkyl-5-6 membered heteroaryl, —O—(C₂-C₆)alkenyl, —O—(C₃-C₈)cycloalkyl, —O-aryl, —O-heteroaryl, —C(O)-aryl, —CO₂H, —CO₂(C₁-C₆)alkyl, —CO₂(C₃-C₈)cycloalkyl, —O₂C(C₁-C₆)alkyl, —O₂C(C₃-C₈)cycloalkyl, or 5-6 membered heteroaryl, wherein any said (C₁-C₆)alkyl, —(C₀-C₆)alkyl-(C₃-C₈)cycloalkyl, —(C₀-C₆)alkyl-aryl, (C₁-C₄)alkoxy, —O—(C₁-C₆)alkyl-(C₃-C₈)cycloalkyl, —O—(C₃-C₈)cycloalkyl, —O-aryl, 5-6 membered heteroaryl, —C(O)-aryl, or 5-6 membered heteroaryl is optionally substituted one, two, or three times by R¹ wherein each R¹ is independently halo, —CN, —SF₅, —OH, (C₁-C₆)alkyl, (C₁-C₄)alkoxy, (C₃-C₈)cycloalkyl, —(C₁-C₆)—OH, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), —O—(C₃-C₈)cycloalkyl, -aryl, 5-6 membered heteroaryl, wherein (C₁-C₆)alkyl, (C₃-C₈)cycloalkyl, aryl, or 5-6 membered heteroaryl is further optionally substituted by one, two, or three substituents independently selected from halogen, (C₁-C₆)alkyl, —(C₁-C₆)alkyl-OH, —(C₁-C₆)alkyl-O—(C₁-C₆)alkyl, or (C₁-C₄)alkoxy;
n is 0 or an integer from 1 to 5;
L¹ is CR^{c}R^{c*}, O, SO₂ or C(O);
each of R^{c} and R^{c*} is independently H, deuterium, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halogen, (C₁-C₆)alkyl, halo(C₁-C₆)alkyl, and (C₃-C₈)cycloalkyl;
Ring A is a 5-membered arylene or heteroarylene group;
each R* is independently selected from hydrogen, halogen, (C₁-C₆)alkyl, halo(C₁-C₆)alkyl, (C₂-C₆)alkenyl, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), (C₂-C₆)alkenyl, —O—(C₁-C₆)alkyl, —NH₂, —NH((C₁-C₆)alkyl), —N((C₁-C₆)alkyl)((C₁-C₆)alkyl), —NCH₂, or —CHNH; or two R* groups taken together with the atoms to which they are attached, form a 5-, 6-, or 7-membered ring optionally containing one, two, or three heteroatoms independently selected from oxygen, nitrogen, and sulfur, wherein said ring is optionally substituted by one or two substituents independently selected from halogen, (C₁—C₆)alkyl, —(C₁-C₆)alkyl-(C₃-C₈)cycloalkyl, halo(C₁-C₆)alkyl, and (C₃-C₈)cycloalkyl;
n* is 0 or an integer from 1 to 3;
or one of R^{c} or R^{c*} and R* taken together with the atom to which they are bound, fuse together to form a 3- to 6-membered ring;
L² is —C(O)— or

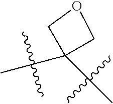 [SMILES: *C1(*)COC1]

wherein the wavy line represents the points of attachment to the NH and the C R^{d}R^{d*} groups;
R^{d} and R^{d*} are each independently H or (C₁-C₃)alkyl, or (C₁-C₃)alkoxy, or R^{d} and R^{d*} cyclize to form a 3 to 6 membered cycloalkyl or heterocycloalkyl ring;
X^{h} and X^{h*} are each independently CH₂, NH, or O;
X^{g} is CH or N;
each R^{c} is independently halo, (C₁-C₃)alkyl, halo(C₁-C₃)alkyl, or (C₁-C₃)alkoxy, or any two R^{c} groups, on the same ring atom or on adjacent ring atoms, cyclize to form a 3 to 6 membered fused or spiro ring;
n** is 0 or an integer from 1 to 8;
X^{e} is N, NH, CH₂ or CH;
X^{f} is SO₂, N, C-(halo(C₁-C₃)alkyl), C—(C₁-C₃)alkyl, C═O or N⁺—O—;
[symbol] represents a double or single bond as needed to satisfy atom valences;
each R^{f} is independently H or halo, (C₁-C₆)alkyl, (C₃-C₈)cycloalkyl, (C₁-C₄)alkoxy, —(C₁-C₆)alkyl-OH, —(C₁-C₆)alkyl-O—(C₁-C₆)alkyl, —(C₁-C₆)alkyl-NH₂, —(C₁-C₆)alkyl-NH((C₁-C₆)alkyl), —(C₁-C₆)alkyl-N—((C₁-C₆)alkyl)((C₁-C₆)alkyl), —(C₁-C₆)alkyl-NH((C₁-C₆)alkyl)-OH, —(C₁-C₆)alkyl-NH((C₁-C₆)alkyl)-O—(C₁-C₆)alkyl), —(C₁-C₆)alkyl-N—((C₁-C₆)alkyl)((C₁-C₆)alkyl)-OH, —(C₁-C₆)alkyl-N—((C₁-C₆)alkyl)((C₁-C₆)alkyl)-O—(C₁-C₆)alkyl), —(C₁-C₆)alkyl-NH—(C₃-C₈)cycloalkyl, —(C₁-C₆)alkyl-NH-4- to 6-membered heterocycloalkyl, —(C₁-C₆)alkyl-4- to 6-membered heterocycloalkyl, —(C₁-C₆)alkyl-NHC(O)—((C₁-C₆)alkyl), —(C₁-C₆)alkyl-SO₂—(C₁-C₆)alkyl, —(C₁-C₆)alkyl-SO₂—NH—(C₁-C₆)alkyl, —CN, —C(O)NH₂, —C(O)NH(C₁-C₆)alkyl), —C(O)N(C₁-C₆)alkyl) (C₁-C₆)alkyl), —NH₂, —NH(C₁-C₆)alkyl, —N((C₁-C₆)alkyl)((C₁-C₆)alkyl), —SO₂((C₁-C₆)alkyl), —SO₂—NH((C₁-C₆)alkyl), or aryl, wherein any said (C₁-C₆)alkyl, (C₃-C₈)cycloalkyl, —(C₁-C₆)alkyl-OH, —(C₁-C₆)alkyl-NH₂, —(C₁-C₆)alkyl-NH((C₁-C₆)alkyl), —(C₁-C₆)alkyl-NH—(C₃-C₈)cycloalkyl, —(C₁-C₆)alkyl-NH-4- to 6-membered heterocycloalkyl, or —(C₁-C₆)alkyl-4- to 6-membered heterocycloalkyl is optionally substituted one, two, or three times by halogen, or any two R^{f} groups, on the same ring atom or on adjacent ring atoms, cyclize to form a 3 to 6 membered fused or spiro ring;
n\hat{ } is 0 or an integer from 1 to 4.

In one aspect, provided here is a compound of Formula (I) or a pharmaceutically acceptable salt thereof:

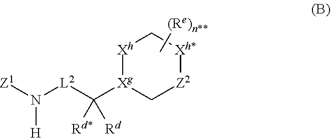

wherein the “*” at the carbon to which R^{d} and R^{d*} are bonded represents a first stereo center;
wherein Z¹ is a monocyclic or bicyclic heteroaryl group substituted with 0, 1, 2 or 3 R groups, or

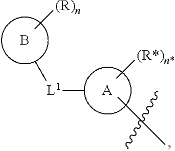

wherein the wavy line represents the point of attachment of the Z¹ group to the NH group;

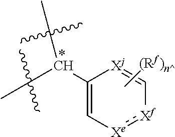

Z² is CH₂ or X^{e} wherein the wavy lines represents the points of attachment of the Z² group to the 6-membered ring and wherein the
“*” at the carbon atom in Z² designates a second stereocenter;
Ring B is a 6-membered aryl or 5- or 6-membered heteroaryl group;
each R is independently selected from hydrogen, CN, SF₅, halogen, (C₁-C₆)alkyl, —(C₀-C₆)alkyl-NH₂, —(C₀-C₆)alkyl-NH((C₁-C₆)alkyl), —(C₀-C₆)alkyl-N((C₁-C₆)alkyl)((C₁-C₆)alkyl), —(C₀-C₆)alkyl-(C₃-C₈)cycloalkyl, —(C₀-C₆)alkyl-aryl, 5- or 6-membered heteroaryl(C₁-C₄)alkyl-, (C₂-C₆)alkenyl, —(C₂-C₆)alkenyl-(C₃-C₈)cycloalkyl, —OH, (C₁-C₄)alkoxy, —O—(C₁-C₆)alkyl-(C₃-C₈)cycloalkyl, —O—(C₁-C₆)alkyl-aryl, —O—(C₁-C₆)alkyl-5-6 membered heteroaryl, —O—(C₂-C₆)alkenyl, —O—(C₃-C₈)cycloalkyl, —O-aryl, —O-heteroaryl, —C(O)-aryl, —CO₂H, —CO₂(C₁-C₆)alkyl, —CO₂(C₃-C₈)cycloalkyl, —O₂C(C₁-C₆)alkyl, —O₂C(C₃-C₈)cycloalkyl, or 5-6 membered heteroaryl, wherein any said (C₁-C₆)alkyl, —(C₀-C₆)alkyl-(C₃-C₈)cycloalkyl, —(C₀-C₆)alkyl-aryl, (C₁-C₄)alkoxy, —O—(C₁-C₆)alkyl-(C₃-C₈)cycloalkyl, —O—(C₃-C₈)cycloalkyl, —O-aryl, 5-6 membered heteroaryl, —C(O)-aryl, or 5-6 membered heteroaryl is optionally substituted one, two, or three times by R¹ wherein each R¹ is independently halo, —CN, —SF₅, —OH, (C₁-C₆)alkyl, (C₁-C₄)alkoxy, (C₃-C₈)cycloalkyl, —(C₁-C₆)—OH, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), —O—(C₃-C₈)cycloalkyl, -aryl, 5-6 membered heteroaryl, wherein (C₁-C₆)alkyl, (C₃-C₈)cycloalkyl, aryl, or 5-6 membered heteroaryl is further optionally substituted by one, two, or three substituents independently selected from halogen, (C₁-C₆)alkyl, —(C₁-C₆)alkyl-OH, —(C₁-C₆)alkyl-O—(C₁-C₆)alkyl, or (C₁-C₄) alkoxy;
n is 0 or an integer from 1 to 5;
L¹ is CR^{c}R^{c*}, O, SO₂ or C(O);
each of R^{c} and R^{c*} is independently H, D, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halogen, (C₁-C₆)alkyl, halo(C₁-C₆)alkyl, and (C₃-C₈)cycloalkyl;
Ring A is a 5-membered arylene or heteroarylene group;
each R* is independently selected from hydrogen, halogen, (C₁-C₆)alkyl, halo(C₁-C₆)alkyl, (C₂-C₆)alkenyl, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), (C₂-C₆)alkenyl, —O—(C₁-C₆)alkyl, —NH₂, —NH((C₁-C₆)alkyl), —N((C₁-C₆)alkyl)((C₁-C₆)alkyl), —NCH₂, or —CHNH; or two R* groups taken together with the atoms to which they are attached, form a 5-, 6-, or 7-membered ring optionally containing one, two, or three heteroatoms independently selected from oxygen, nitrogen, and sulfur, wherein said ring is optionally substituted by one or two substituents independently selected from halogen, (C₁-C₆)alkyl, —(C₁-C₆)alkyl-(C₃-C₈)cycloalkyl, halo(C₁-C₆)alkyl, and (C₃-C₈)cycloalkyl;
n* is 0 or an integer from 1 to 3;
or one of R^{c} or R^{c*} and R* taken together with the atom to which they are bonded, fuse together to form a 3- to 6-membered ring;
L² is —C(O)— or

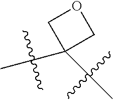 [SMILES: *C1(*)COC1]

wherein the wavy line represents the points of attachment to the NH and the C R^{d}R^{d*} groups;
R^{d} and R^{d*} are each independently H or (C₁-C₃)alkyl, or (C₁-C₃)alkoxy, or R^{d} and R^{d*} cyclize to form a 3 to 6 membered cycloalkyl or heterocycloalkyl ring;
X^{h} and X^{h*} are each independently C(R^{e})₂, N(R^{e}), CH₂, NH, or O;
X^{g} is CH or N;
each R^{e} is independently H, halo, (C₁-C₃)alkyl, halo(C₁-C₃)alkyl, or (C₁-C₃)alkoxy, or any two R^{e} groups, on the same ring atom or on adjacent ring atoms, cyclize to form a 3 to 6 membered fused or spiro ring;
n** is 0 or an integer from 1 to 8;
X^{j} is N, NR^{f}, C(R^{f})₂, C(R^{f}) or CH; X^{e} is N, NR^{f}, C(R^{f})₂ or C(R^{f});
X^{f} is SO₂, N, C-(halo(C₁-C₃)alkyl), C—(C₁-C₃)alkyl, C═O or N⁺—O—;
[symbol] represents a double or single bond as needed to satisfy atom valences;
each R^{f} is independently H or halo, (C₁-C₆)alkyl, (C₃-C₈)cycloalkyl, (C₁-C₄)alkoxy, —(C₁-C₆)alkyl-OH, —(C₁-C₆)alkyl-O—(C₁-C₆)alkyl, —(C₁-C₆)alkyl-NH₂, —(C₁-C₆)alkyl-NH((C₁-C₆)alkyl), —(C₁-C₆)alkyl-N—((C₁-C₆)alkyl)((C₁-C₆)alkyl), —(C₁-C₆)alkyl-NH((C₁-C₆)alkyl)-OH, —(C₁-C₆)alkyl-NH((C₁-C₆)alkyl)-O—(C₁-C₆)alkyl), —(C₁-C₆)alkyl-N—((C₁-C₆)alkyl)((C₁-C₆)alkyl)-OH, —(C₁-C₆)alkyl-N—((C₁-C₆)alkyl)((C₁-C₆)alkyl)-O—(C₁-C₆)alkyl), —(C₁-C₆)alkyl-NH—(C₃-C₈)cycloalkyl, —(C₁-C₆)alkyl-NH-4- to 6-membered heterocycloalkyl, —(C₁-C₆)alkyl-4- to 6-membered heterocycloalkyl, —(C₁-C₆)alkyl-NHC(O)—((C₁-C₆)alkyl), —(C₁-C₆)alkyl-SO₂—(C₁-C₆)alkyl, —(C₁-C₆)alkyl-SO₂—NH—(C₁-C₆)alkyl, —CN, —C(O)NH₂, —C(O)NH(C₁-C₆)alkyl), —C(O)N(C₁-C₆)alkyl) (C₁-C₆)alkyl), —NH₂, —NH(C₁-C₆)alkyl, —N((C₁-C₆)alkyl)((C₁-C₆)alkyl), —SO₂((C₁-C₆)alkyl), —SO₂—NH((C₁-C₆)alkyl), or aryl, wherein any said (C₁-C₆)alkyl, (C₃-C₈)cycloalkyl, —(C₁-C₆)alkyl-OH, —(C₁-C₆)alkyl-NH₂, —(C₁-C₆)alkyl-NH((C₁-C₆)alkyl), —(C₁-C₆)alkyl-NH—(C₃-C₈)cycloalkyl, —(C₁-C₆)alkyl-NH-4- to 6-membered heterocycloalkyl, or —(C₁-C₆)alkyl-4- to 6-membered heterocycloalkyl is optionally substituted one, two, or three times by halogen, or any two R^{f} groups, on the same ring atom or on adjacent ring atoms, cyclize to form a 3 to 6 membered fused or spiro ring;
n\hat{ } is 0 or an integer from 1 to 4.

In one aspect, provided here is a compound of Formula (I) or a pharmaceutically acceptable salt thereof:

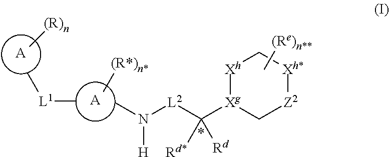

wherein the “*” at the carbon to which R^{d} and R^{d*} are bonded is a first stereo center when R^{d} and R^{d*} are different;

Z² is CH₂ or

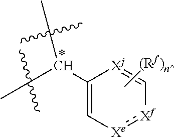

wherein the wavy lines represent the points of attachment of the Z² group to the X^{h*} and the —CH₂ of the 6-membered ring;
wherein the “*” at the carbon atom in Z² designates a second stereocenter;
with the proviso that when Z² is —CH₂, then Ring A is not a thiazolyl;
Ring B is a 6-membered aryl or 5- or 6-membered heteroaryl group;
each R is independently selected from hydrogen, CN, SF₅, halo, (C₁-C₆)alkyl, —(C₀-C₆)alkyl-NH₂, —(C₀-C₆)alkyl-NH((C₁-C₆)alkyl), —(C₀-C₆)alkyl-N((C₁-C₆)alkyl)((C₁-C₆)alkyl), —(C₀-C₆)alkyl-(C₃-C₈)cycloalkyl, —(C₀-C₆)alkyl-aryl, 5- or 6-membered heteroaryl(C₁-C₄)alkyl-, (C₂-C₆)alkenyl, —(C₂-C₆)alkenyl-(C₃-C₈)cycloalkyl, —OH, (C₁-C₄)alkoxy, —O—(C₁-C₆)alkyl-(C₃-C₈)cycloalkyl, —O—(C₁-C₆)alkyl-aryl, —O—(C₁-C₆)alkyl-5-6 membered heteroaryl, —O—(C₂-C₆)alkenyl, —O—(C₃-C₈)cycloalkyl, —O-aryl, —O-heteroaryl, —C(O)-aryl, —CO₂H, —CO₂(C₁-C₆)alkyl, —CO₂(C₃-C₈)cycloalkyl, —O₂C(C₁-C₆)alkyl, —O₂C(C₃-C₈)cycloalkyl, or 5-6 membered heteroaryl, wherein any said (C₁-C₆)alkyl, —(C₀-C₆)alkyl-(C₃-C₈)cycloalkyl, —(C₀-C₆)alkyl-aryl, (C₁-C₄)alkoxy, —O—(C₁-C₆)alkyl-(C₃-C₈)cycloalkyl, —O—(C₃-C₈)cycloalkyl, —O-aryl, 5-6 membered heteroaryl, —C(O)-aryl, or 5-6 membered heteroaryl is optionally substituted one, two, or three times by R¹ wherein each R¹ is independently halo, —CN, —SF₅, —OH, (C₁-C₆)alkyl, (C₁-C₄)alkoxy, (C₃-C₈)cycloalkyl, —(C₁-C₆)—OH, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), —O—(C₃-C₈)cycloalkyl, -aryl, 5-6 membered heteroaryl, wherein (C₁-C₆)alkyl, (C₃-C₈)cycloalkyl, aryl, or 5-6 membered heteroaryl is further optionally substituted by one, two, or three substituents independently selected from halo, (C₁-C₆)alkyl, —(C₁-C₆)alkyl-OH, —(C₁-C₆)alkyl-O—(C₁-C₆)alkyl, or (C₁-C₄)alkoxy;
n is 0 or an integer from 1 to 5;
L¹ is C(R^{c})(R^{c*}), O, SO₂ or C(O);
each of R^{c} and R^{c*} is independently H, D, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halo, (C₁-C₆)alkyl, halo(C₁-C₆)alkyl, and (C₃-C₈)cycloalkyl;
Ring A is a 5-membered arylene or a 5-membered heteroarylene group;
each R* is independently selected from hydrogen, OH, halo, (C₁-C₆)alkyl, halo(C₁-C₆)alkyl, (C₂-C₆)alkenyl, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), (C₂-C₆)alkenyl, —O—(C₁-C₆)alkyl, —NH₂, —NH((C₁-C₆)alkyl), —N((C₁-C₆)alkyl)((C₁-C₆)alkyl), —NCH₂, or —CHNH; or two R* groups taken together with the atoms to which they are attached, form a 5-, 6-, or 7-membered ring optionally containing one, two, or three heteroatoms independently selected from oxygen, nitrogen, and sulfur, wherein said ring is optionally substituted by one or two substituents independently selected from halo, (C₁-C₆)alkyl, —(C₁-C₆)alkyl-(C₃-C₈)cycloalkyl, halo(C₁-C₆)alkyl, and (C₃-C₈)cycloalkyl;
n* is 0 or an integer from 1 to 3;
or one of R^{c} or R^{c*} and R* taken together with the atoms to which they are bonded form a 3- to 6-membered ring fused to Ring A;

L² is —C(O)— or

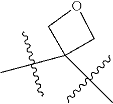 [SMILES: *C1(*)COC1]

wherein the wavy line represents the points of attachment to the NH and the C(R^{d})(R^{d*}) groups;
R^{d} and R^{d*} are each independently H or (C₁-C₃)alkyl, or (C₁-C₃)alkoxy, or R^{d} and R^{d*} cyclize to form a 3 to 6 membered cycloalkyl or heterocycloalkyl ring;
X^{h} and X^{h*} are each independently C(R^{e})₂, N(R^{e}), or 0;
X^{g} is CH or N;
each R^{e} is independently H, halo, (C₁-C₃)alkyl, halo(C₁-C₃)alkyl, or (C₁-C₃)alkoxy, or any two R^{e} groups, on the same ring atom or on adjacent ring atoms, cyclize to form a 3 to 6 membered fused or spiro ring;
n** is 0 or an integer from 1 to 8;
X^{j} is N, NR^{f}, C(R^{f})₂, C(R^{f}) or CH;
X^{e} is N, NR^{f}, C(R^{f})₂ or C(R^{f});
X^{f} is SO₂, N, C(halo(C₁-C₃)alkyl), C—(C₁-C₃)alkyl, C═O or N⁺—O—;
[symbol] represents a double or single bond as needed to satisfy atom valences;
each R^{f} is independently H or halo, (C₁-C₆)alkyl, (C₃-C₈)cycloalkyl, (C₁-C₄)alkoxy, —(C₁-C₆)alkyl-OH, —(C₁-C₆)alkyl-O—(C₁-C₆)alkyl, —(C₁-C₆)alkyl-NH₂, —(C₁-C₆)alkyl-NH((C₁-C₆)alkyl), —(C₁-C₆)alkyl-N—((C₁-C₆)alkyl)((C₁-C₆)alkyl), —(C₁-C₆)alkyl-NH((C₁-C₆)alkyl)-OH, —(C₁-C₆)alkyl-NH((C₁-C₆)alkyl)-O—(C₁-C₆)alkyl), —(C₁-C₆)alkyl-N—((C₁-C₆)alkyl)((C₁-C₆)alkyl)-OH, —(C₁-C₆)alkyl-N—((C₁-C₆)alkyl)((C₁-C₆)alkyl)-O—(C₁-C₆)alkyl), —(C₁-C₆)alkyl-NH—(C₃-C₈)cycloalkyl, —(C₁-C₆)alkyl-NH-4- to 6-membered heterocycloalkyl, —(C₁-C₆)alkyl-4- to 6-membered heterocycloalkyl, —(C₁-C₆)alkyl-NHC(O)—((C₁-C₆)alkyl), —(C₁-C₆)alkyl-SO₂—(C₁—C₆)alkyl, —(C₁-C₆)alkyl-SO₂—NH—(C₁-C₆)alkyl, —CN, —C(O)NH₂, —C(O)NH(C₁-C₆)alkyl), —C(O)N(C₁-C₆)alkyl) (C₁-C₆)alkyl), —NH₂, —NH(C₁-C₆)alkyl, —N((C₁-C₆)alkyl)((C₁-C₆)alkyl), —SO₂((C₁-C₆)alkyl), —SO₂—NH((C₁-C₆)alkyl), or aryl, wherein any said (C₁-C₆)alkyl, (C₃-C₈)cycloalkyl, —(C₁-C₆)alkyl-OH, —(C₁-C₆)alkyl-NH₂, —(C₁-C₆)alkyl-NH((C₁-C₆)alkyl), —(C₁-C₆)alkyl-NH—(C₃-C₈)cycloalkyl, —(C₁-C₆)alkyl-NH-4- to 6-membered heterocycloalkyl, or —(C₁-C₆)alkyl-4- to 6-membered heterocycloalkyl is optionally substituted one, two, or three times by halo, OH, or any two R groups, on the same ring atom or on adjacent ring atoms, cyclize to form a 3 to 6 membered fused or spiro ring;
n\hat{ } is 0 or an integer from 1 to 4.

In some embodiments, Ring A is imidazolylene, oxazolylene, oxadiazolylene, pyrazolylene, pyrrolylene, thiazolylene, or triazolylene. In some embodiments, Ring A is pyrrolylene, thiazolylene, imidazolylene, pyrazolylene, or triazolylene. In some embodiments, Ring A is imidazolylene. In some embodiments, Ring A is

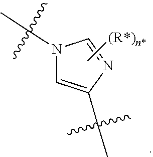

In some embodiments, Ring A is imidazolylene, triazolylene, pyrazolylene, or thiazolylene; n* is 0, 1 or 2; L¹ is C(R^{c})(R^{c*}) and one of R^{c} or R^{c*} and R* taken together with the atoms to which they are bonded form an optionally substituted 3- to 6-membered ring fused to Ring A. In some embodiments, the optionally substituted 3- to 6-membered ring fused to Ring A forms an optionally substituted 8- or 9-membered bicyclic heterocyclylene having 1, 2 or 3 heteroatoms selected from N and S, or an optionally substituted 8- or 9-membered bicyclic heteroarylene having 1, 2 or 3 heteroatoms selected from N and S. In some embodiments, Ring A forms 6,7-dihydro-5H-pyrrolo[1,2-a]imidazol-2-ylene.

In some embodiments, the optionally substituted 3- to 6-membered ring fused to Ring A has the structure

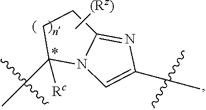

wherein R^{z} is absent or is OH, n′ is 0, 1 or 2, and the at the ring carbon bonded to Ring B represents a third stereocenter. In some embodiments, n′ is 0, or is 1, or is 2. In some embodiments, n′ is 1. In some embodiments, n′ is 2.

In some embodiments, the 3- to 6-membered ring fused to Ring A has a structure selected from:

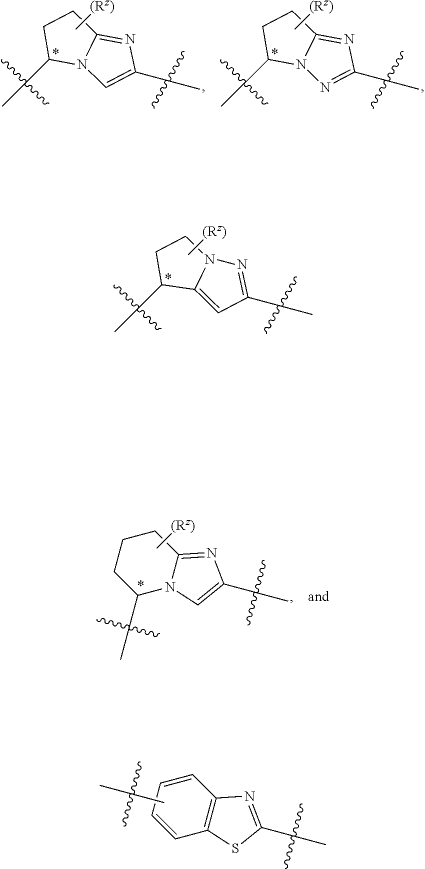

wherein R^{z} is absent or is OH, and the “*” at the ring carbon bonded to Ring B represents a third stereocenter. In some embodiments, R^{z} is absent. In some embodiments, R^{z} is OH.

In some embodiments, X^{g} is CH. In some embodiments, X^{g} is N.

In some embodiments, R^{d} and R^{d*} cyclize to form a 3 to 6 membered ring. In some embodiments, R^{d} and R^{d*} are the same and are H or (C₁-C₃)alkyl. In some embodiments, at least one of R^{d} and R^{d*} is (C₁-C₃)alkoxy. In some embodiments, each R^{d} and R^{d*} are independently chosen from H and CH₃.

In some embodiments, R^{d} and R^{d*} are different and Z² is

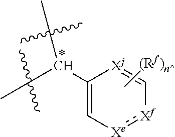

and the first and second stereocenters are both S.

In some embodiments, R^{d} and R^{d*} are different and Z² is

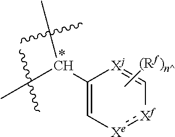

and the first and second stereocenters are both R, or the first stereocenter is S and second stereocenter is R, or the first stereocenter is R and second stereocenter is S.

In some embodiments, two R^{e} groups on adjacent atoms cyclize to form a 3 to 6 membered ring. In some embodiments, two R^{e} groups bonded to a single carbon atom cyclize to form a 3 to 6 membered ring. In some embodiments, at least one R^{e} is halo, (C₁-C₃)alkyl, or (C₁-C₃)alkoxy. In some embodiments, each R^{e} is independently chosen from F, CH₃ and OCH₃.

In some embodiments, X^{h*} is CH₂. In some embodiments, X^{h*} is C(R^{e})₂ and each R^{e} is F.

In some embodiments, n** is 1, 2, 3, or 4. In some embodiments, n** is 1 or 2. In some embodiments, n** is 0. In some embodiments, n** is 1. In some embodiments, n** is 2. In some embodiments, n** is 3. In some embodiments, n** is 4.

In some embodiments, X^{h} is NH or O. In some embodiments, X^{h} is CH₂. In some embodiments, X^{h*} is NH, NCH₃, or O. In some embodiments, X^{h*} is CH₂, C(R^{e})₂, or CH(R^{e}).

In some embodiments, L² is

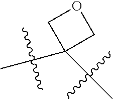 [SMILES: *C1(*)COC1]

wherein the wavy line represents the points of attachment to the NH and the C(R^{d})(R^{d*}) groups. In some embodiments, L² is C═O.

In some embodiments, R* is CH₃, or CF₃.

In some embodiments, n* is 1 or 2. In some embodiments, n* is 1. In some embodiments, n* is 0.

In some embodiments, Ring B is phenyl, pyridinyl, pyridazinyl, or pyrimidinyl. In some embodiments, Ring B is phenyl or pyridinyl. In some embodiments, Ring B is phenyl.

In some embodiments, each R is independently CN, SF₅, Cl, F, CH₃, OCH₃, O-phenyl, CF₃ or OCF₃. In some embodiments, each R is independently F, Cl, CN, CF₃ or OCF₃.

In some embodiments, n is 1, 2 or 3. In some embodiments, n is 1 or 2. In some embodiments, n is 0. In some embodiments, n is 1. In some embodiments, n is 2. In some embodiments, n is 3.

In some embodiments, n\hat{ } is 1 or 2. In some embodiments, n\hat{ } is 0. In some embodiments, n\hat{ } is 1. In some embodiments, n\hat{ } is 2.

In some embodiments, L¹ is O, CH₂, CHCH₃, or CD₂. In some embodiments, L¹ is O. In some embodiments, L¹ is CH₂ or CD₂. In some embodiments, L¹ is CH₂. In some embodiments, L¹ is absent. In some embodiments, L¹ is C(R^{c})(R^{c*}).

In some embodiments, L¹ is C(R^{c})(R^{c*}) and each R^{c} and R^{c*} is independently chosen from H, D and CH₃. In some embodiments, at least one of R^{c} and R^{c*} is (C₁-C₃)alkoxy. In some embodiments, at least one of R^{c} and R^{c*} is methoxy or ethoxy. In some embodiments, R^{c} and R^{c*} are the same and are H, D, or (C₁-C₃)alkyl. In some embodiments, R^{c} and R^{c*} cyclize to form a 3 to 6 membered ring.

In some embodiments, L¹ is C(R^{c})(R^{c*}) and one of R^{c} or R^{c*} and R* taken together with the atoms to which they are bonded form a 3- to 6-membered ring fused to Ring A. In some embodiments, the 3- to 6-membered ring fused to Ring A has the structure

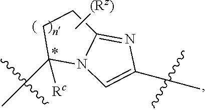

wherein R^{z} is absent or is OH, n′ is 0, 1 or 2, and the “*” at the ring carbon bonded to R^{c*} represents a third stereocenter. In some embodiments, n′ is 1 or 2. In some embodiments, n′ is 1. In some embodiments, n′ is 2.

In some embodiments, the 3- to 6-membered ring fused to Ring A has a structure selected from

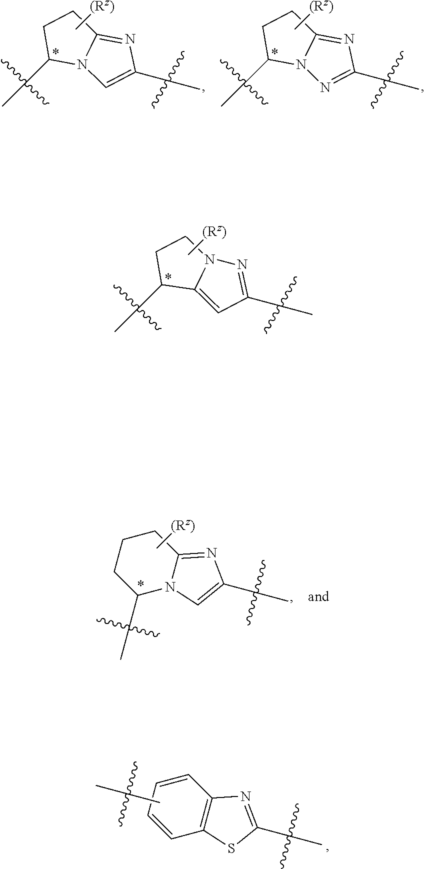

wherein the ring carbon attached to Ring B is a stereocenter. In some embodiments, the 3- to 6-membered ring fused to Ring A forms a group having a structure selected from

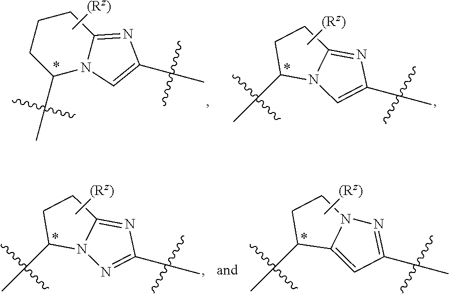

wherein R^{z} is absent or is OH, and the “*” at the ring carbon bonded to R^{c*} represents a third stereocenter.

In some embodiments, R^{z} is absent. In some embodiments, R^{z} is OH.

In some embodiments, X^{e} is NH and X^{f} is C═O and the bond between X^{e} and X^{f} is a single bond. In some embodiments, X^{e} is CH₂ and X^{f} is C═O and the bond between X^{e} and X^{f} is a single bond. In some embodiments, X^{e} is CH and X^{f} is N⁺—O⁻ and the bond between X^{e} and X^{f} is a double bond. In some embodiments, X^{e} is CH and X^{f} is N and the bond between X^{e} and X^{f} is a double bond. In some embodiments, X^{e} is N and X^{f} is C—CH₃, C—CH₂F, C—CHF₂. or CF₃ and the bond between X^{e} and X^{f} is a double bond.

In some embodiments, two R^{f} groups on adjacent atoms cyclize to form a 3 to 6 membered ring. In some embodiments, two R^{f} groups bonded to a single carbon atom cyclize to form a 3 to 6 membered ring. In some embodiments, each R^{f} is independently chosen from halo, (C₁-C₃)alkyl, (C₁-C₃)alkyl-OH, halo(C₁-C₃)alkyl, (C₁-C₃)alkoxy, or aryloxy. In some embodiments, each R^{f} is independently chosen from CH₂—OH, F, Cl, CH₃, OCH₃, O-phenyl, CF₃, and OCF₃.

In some embodiments, Z¹ is a monocyclic or bicyclic heteroaryl group. In some embodiments, Z¹ is [1,3]dioxolo[4′,5′:4,5]benzo[1,2-d]thiazolylene. In some embodiments, Z¹ is

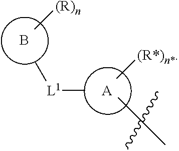

In some embodiments, Z¹ is

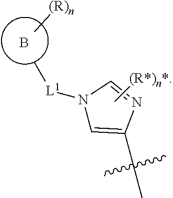

In some embodiments, Z² is CH₂. In some embodiments, Z² is

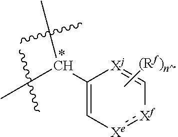

wherein the wavy lines represent the points of attachment of the Z² group to the 6-membered ring In some embodiments, Z² is

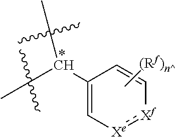

wherein the wavy lines represent the points of attachment of the Z² group to the 6-membered ring.

In some embodiments, Z¹ is a monocyclic or bicyclic heteroaryl group. In some embodiments, Z¹ is pyridinyl or pyrimidinyl.

In some embodiments, Z¹ is

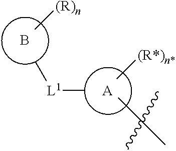

### and Z² is

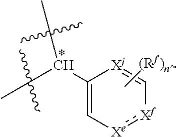

In some embodiments, Z¹ is

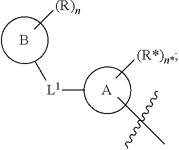

ring A is imidazolylene; L² is —C(O)—; X^{h} is NH; X^{h*} and X^{g} are CH₂; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, n is 2 and R is halo.

In some embodiments, Z¹ is

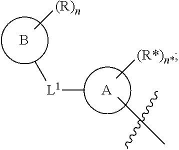

ring A is imidazolylene; L² is —C(O)—; X^{g} is N; X^{h} and X^{h*}, are CH₂; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, n is 2 and R is halo.

In some embodiments, Z¹ is

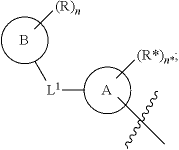

ring A is imidazolylene; L² is —C(O)—; Z² is

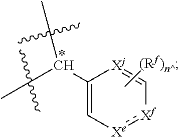

X^{h*} is O; X^{h} is CH₂; X^{g} is CH; X^{e} is CH; X^{f} is N⁺—O⁻ and the bond between X^{e} and X^{f} is a double bond; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, R^{f} is CH₃, OCH₃, CF₃, and OCF₃. In some embodiments, n is 2 and R is halo.

In some embodiments, Z¹ is

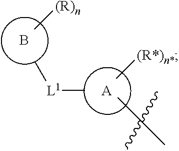

ring A is imidazolylene; L² is —C(O)—; Z² is

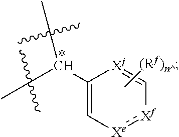

X^{h*} is O; X^{h} is CH₂; X^{g} CH; X^{e} is NH; X^{f} is C═O and the bond between X^{e} and X^{f} is a single bond; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, R^{f} is CH₃, OCH₃, CF₃, and OCF₃. In some embodiments, n is 2 and R is halo.

In some embodiments, Z¹ is

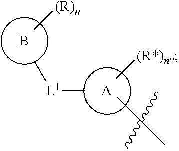

ring A is imidazolylene; L² is —C(O)—; Z² is

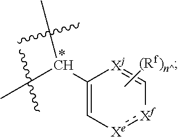

X^{g} is N; X^{h} and X^{h*} are CH₂; X^{h} is CH; X^{h*} is N⁺—O⁻ and the bond between X^{e} and X is a double bond; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, R^{f} is CH₃, OCH₃, CF₃, and OCF₃. In some embodiments, n is 2 and R is halo.

In some embodiments, Z¹ is

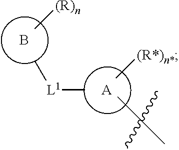

ring A is imidazolylene; L² is —C(O)—; Z² is

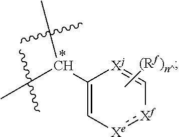

X^{g} is N; X^{h} and X^{h*} are CH₂; X^{e} is NH; X^{f} is C═O and the bond between X^{e} and X^{f} is a single bond; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, R^{f} is CH₃, OCH₃, CF₃, and OCF₃. In some embodiments, n is 2 and R is halo.

In some embodiments, Z¹ is

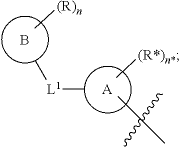

Z² is CH₂; L² is —C(O)—; L¹ is methylene; X^{g} is N; X^{h} and X^{h*} are C. In some embodiments, Ring A is imidazolylene; and R^{d*} is CH₃. In some embodiments, R^{e} is halo or (C₁-C₃)alkyl; B is phenyl; and R is independently selected from halo and (C₁-C₃)alkyl. In some embodiments, R is halo and n is 2.

In some embodiments, Z¹ is

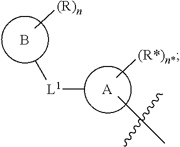

Z² is CH₂; L² is —C(O)—; L¹ is methylene; X^{g} is N; X^{h} is CH₂; and X^{h*} is O. In some embodiments, Ring A is imidazolylene; and R^{d*} is CH₃. In some embodiments, R^{e} is halo or (C₁-C₃)alkyl; B is phenyl; and R is independently selected from halo and (C₁-C₃)alkyl. In some embodiments, R is halo and n is 2.

In some embodiments, Z¹ is a monocyclic or bicyclic heteroaryl group substituted with 0, 1 or 2 R groups; Z² is CH₂; L² is —C(O)—; X^{g} is N; X^{h} and X^{h*} are C. In some embodiments, R^{d*} is CH₃. In some embodiments, each R^{e} is independently H, halo or (C₁-C₃)alkyl; and each R is independently selected from halo and (C₁-C₃)alkyl. In some embodiments, R is fluoro.

In some embodiments, Z¹ is a monocyclic or bicyclic heteroaryl group substituted with 0, 1 or 2 R groups; Z² is CH₂; L² is —C(O)—; X^{g} is N; X^{h} is CH₂; and X^{h*} is O. In some embodiments, R^{d*} is CH₃. In some embodiments, each R^{e} is independently H, halo or (C₁-C₃)alkyl; and each R is independently selected from halo and (C₁-C₃)alkyl. In some embodiments, R is fluoro.

In some embodiments, Z¹ is a monocyclic or bicyclic heteroaryl group substituted with 0, 1 or 2 R groups; Z² is

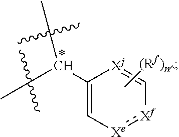

X^{h*} is O; X^{h} is CH₂; and X^{g} CH; X^{e} is CH; X^{f} is N⁺—O⁻ and the bond between X^{e} and X^{f} is a double bond; and R^{d*} is CH₃. In some embodiments, R^{d} is H; and each R is independently selected from halo and (C₁-C₃)alkyl. In some embodiments, R^{f} is CH₃, CH₂—OH, OCH₃, CF₃, and OCF₃; and R is fluoro.

In some embodiments, Z¹ is a monocyclic or bicyclic heteroaryl group substituted with 0, 1 or 2 R groups; Z² is

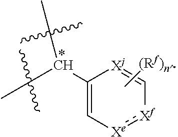

X^{h*} is O; X^{h} is CH₂; and X^{g} is CH; X^{e} is NH; X^{f} is C═O and the bond between X^{e} and X^{f} is a single bond; and R^{d*} is CH₃. In some embodiments, R^{d} is H; n is 0, 1, or 2; and each R is independently selected from halo and (C₁-C₃)alkyl. In some embodiments, R^{f} is CH₃, OCH₃, CH₂—OH, CF₃, and OCF₃; and R is fluoro.

In some embodiments, Z¹ is

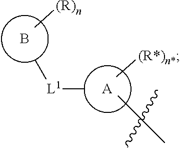

ring A is imidazolylene; L² is —C(O)—; Z² is

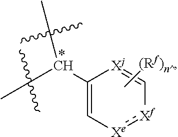

X^{g} is N; X^{h} and X^{h*} are CH₂; X^{e} is CH; X^{f} is N⁺—O⁻ and the bond between X^{e} and X^{f} is a double bond; and R^{d*} is CH₃. In some embodiments, L¹ is methylene; R^{d} is H; and B is phenyl; n is 0, 1, 2, or 3; and each R is independently selected from halo and (C₁-C₃)alkyl. In some embodiments, R^{f} is CH₃, OCH₃, CH₂—OH, CF₃, and OCF₃; and R is fluoro.

In some embodiments, Z¹ is

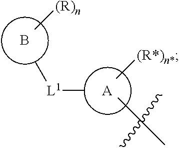

ring A is imidazolylene; L² is —C(O)—; Z² is

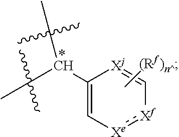

X^{g} is N; X^{h} and X^{h*} are CH₂; X^{e} is NH; X^{f} is C═O and the bond between X^{e} and X^{f} is a single bond; and R^{d*} is CH₃. In some embodiments, R^{d} is H; and each R is independently selected from halo and (C₁-C₃)alkyl. In some embodiments, R^{f} is CH₃, OCH₃, CH₂—OH, CF₃, and OCF₃; and R is fluoro.

In some embodiments, one of R^{c} or R^{c*} and R* taken together with the atoms to which they are bonded form a 3- to 6-membered ring fused to Ring A; Z² is CH₂; L² is —C(O)—; X^{g} is N; X^{h} is CH₂; and X^{h*} is O. In some embodiments, R^{d*} is CH₃. In some embodiments, each R^{c} is independently H, halo or (C₁-C₃)alkyl; and each R is independently selected from halo and (C₁-C₃)alkyl. In some embodiments, R is fluoro.

In some embodiments, one of R^{c} or R^{c*} and R* taken together with the atoms to which they are bonded form a 3- to 6-membered ring fused to Ring A; Z² is

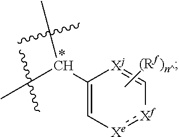

X^{h*} and X^{h} are CH₂; X^{g} is N; X^{e} is CH; X^{f} is N⁺—O⁻ and the bond between X^{e} and X^{f} is a double bond; and R^{d*} is CH₃. In some embodiments, R^{c} or R^{c*} and R* taken together with the atoms to which they are bonded fuse together with Ring A to form an 8 membered bicyclic heteroaryl or heterocyclic group having two nitrogen atoms. In some embodiments, the 8 membered bicyclic heteroaryl or heterocyclic group having two nitrogen atoms comprises a 5 membered heteroaryl group fused to a 5 membered heterocyclic group. In some embodiments, 8 membered bicyclic heteroaryl or heterocyclic group having two nitrogen atoms is 6,7-dihydro-5H-pyrrolo[1,2-a]imidazolylene In some embodiments, R^{d} is H; and each R is independently selected from halo and (C₁-C₃)alkyl. In some embodiments, wherein R^{c} is fluoro and n** is 2. In some embodiments, R^{f} is H.

In some embodiments, one of R^{c} or R^{c*} and R* taken together with the atoms to which they are bonded form a 3- to 6-membered ring fused to Ring A; Z² is

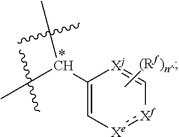

X^{h*} and X^{h} are CH₂; X^{g} is N; X^{e} is N; X^{f} is C(O) and the bond between X^{e} and X^{f} is a single bond; and R^{d*} is CH₃. In some embodiments, R^{c} or R^{c*} and R* taken together with the atoms to which they are bonded fuse together with Ring A to form an 8 membered bicyclic heteroaryl or heterocyclic group having two nitrogen atoms. In some embodiments, the 8 membered bicyclic heteroaryl or heterocyclic group having two nitrogen atoms comprises a 5 membered heteroaryl group fused to a 5 membered heterocyclic group. In some embodiments, the bicyclic ring is 6,7-dihydro-5H-pyrrolo[1,2-a]imidazolylene. In some embodiments, R^{d} is H; and each R is independently selected from halo and (C₁-C₃)alkyl. In some embodiments, R^{c} is fluoro and n** is 2. In some embodiments, R^{f} is H.

In some embodiments, R^{c} or R^{c*} and R* taken together with the atoms to which they are bonded fuse together with Ring A to form an 8 membered bicyclic heteroaryl or heterocyclic group having two nitrogen atoms. In some embodiments, B is phenyl, pyridinyl, pyridazinyl or pyrimidinyl and each R is independently F, Cl, CN, CF₃ or OCF₃ and n is 2 In some embodiments, B is phenyl and each R is F and n is 2. In some embodiments, R^{d} and R^{d*} are different and wherein Z² is

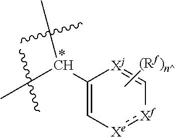

and wherein the first and second stereocenters are both S.

In some embodiments, R^{c} or R^{c*} and R* taken together with the atoms to which they are bonded fuse together with Ring A to form an 8 membered bicyclic heteroaryl or heterocyclic group having two nitrogen atoms, wherein the 8 membered bicyclic heteroaryl or heterocyclic group having two nitrogen atoms is 6,7-dihydro-5H-pyrrolo[1,2-a]imidazolylene. In some embodiments, B is phenyl, pyridinyl, pyridazinyl or pyrimidinyl and each R is independently F, Cl, CN, CF₃ or OCF₃ and n is 2. In some embodiments, B is phenyl and each R is F and n is 2. In some embodiments, R^{d} and R^{d*} are different, wherein Z² is

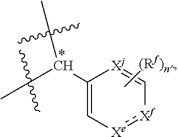

and wherein the first and second stereocenters are both S.

In some embodiments, the compound is a compound of Formula (I), wherein L¹ is C(R^{c})(R^{c*}) and one of R^{c} and R^{c*}, and R* taken together with the atoms to which they are bonded form a 3- to 6-membered ring fused to Ring A; Z² is CH₂; L² is —C(O)—; X^{g} is N; X^{h} is CH₂ and X^{h*} is O. In some embodiments, the 3- to 6-membered ring fused to Ring A forms a group having a structure selected from

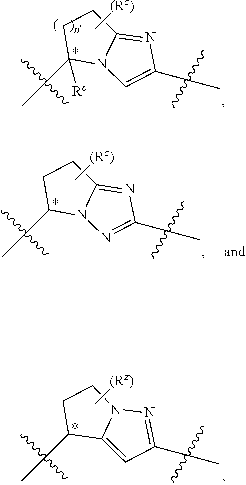

wherein R^{z} is absent or OH, n′ is 1 or 2, and the “*” at the ring carbon bonded to Ring B represents a third stereocenter. In some embodiments, the 3- to 6-membered ring fused to Ring A forms a group having a structure selected from

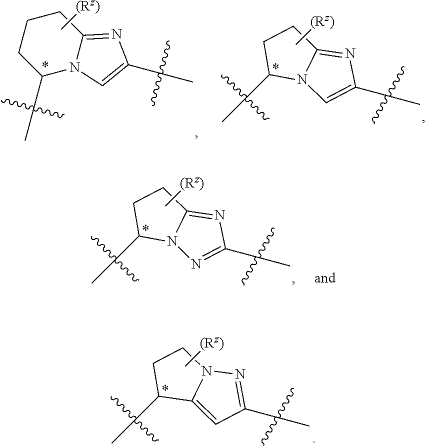

In some embodiments, R^{z} is absent and Ring B is phenyl or pyridinyl. In some embodiments, R^{d*} is CH₃. In some embodiments, n** is 1 or 2 and each R is independently H, halo or (C₁-C₃)alkyl; and each R is independently selected from halo and (C₁-C₃)alkyl. In some embodiments, n is 0, 1, 2, or 3 and R is fluoro.

In some embodiments, the compound is a compound of Formula (I), wherein L¹ is C(R^{c})(R^{c*}) and one of R^{c} or R^{c*}, and R* taken together with the atoms to which they are bonded form a 3- to 6-membered ring fused to Ring A; Z² is CH₂; L² is —C(O)—; X^{g} is N; X^{h} is CH₂; X^{h*} is CH₂ or C(R^{e})₂ and each R^{c} is independently halo or (C₁-C₃)alkyl; and each R is independently selected from halo and (C₁-C₃)alkyl. In some embodiments, the 3- to 6-membered ring fused to Ring A forms a group having a structure selected from

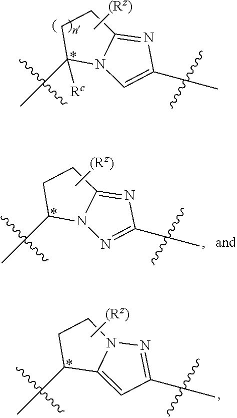

wherein R^{z} is absent or OH, n′ is 1 or 2, and the “*” at the ring carbon bonded to Ring B represents a third stereocenter. In some embodiments, the 3- to 6-membered ring fused to Ring A forms a group having a structure selected from

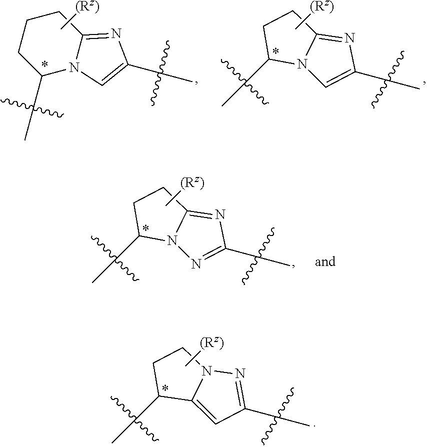

In some embodiments, R^{z} is absent and Ring B is phenyl or pyridinyl. In some embodiments, R^{d*} is CH₃. In some embodiments, n** is 1 or 2 and each R^{c} is independently H, F or Cl. In some embodiments, n is 0, 1, 2, or 3 and R is fluoro.

In some embodiments, the compound is a compound of Formula (I), wherein L¹ is C(R^{c})(R^{c*}) and one of R^{c} or R^{c*} and R* taken together with the atoms to which they are bonded form a 3- to 6-membered ring fused to Ring A; Z² is

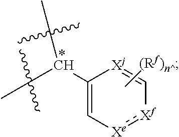

X^{h} is CH₂; X^{h*} is CH₂ or C(R^{c})₂; X^{g} is N; X^{e} is CH; X^{f} is N⁺—O⁻ and the bond between X^{e} and X^{f} is a double bond; and R^{d*} is CH₃. In some embodiments, the 3- to 6-membered ring fused to Ring A forms a group having a structure selected from

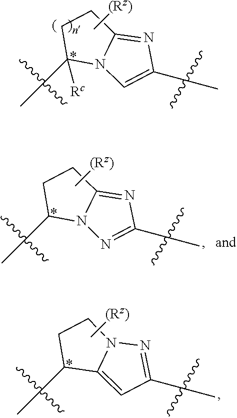

wherein R^{z} is absent or OH, n′ is 1 or 2, and the “*” at the ring carbon bonded to Ring B represents a third stereocenter. In some embodiments, the 3- to 6-membered ring fused to Ring A forms a group having a structure selected from

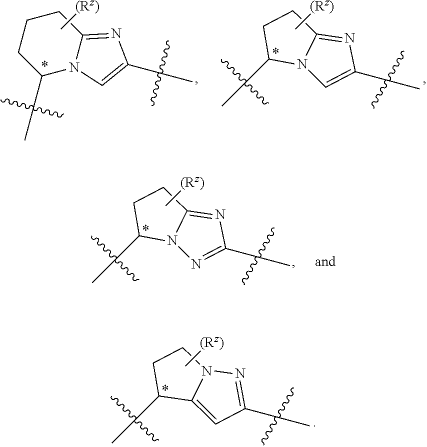

In some embodiments, R^{c} is F and n** is 2. In some embodiments, R^{f} is H. In some embodiments, R^{z} is absent and Ring B is phenyl or pyridinyl. In some embodiments, R^{d} is H; and each R is independently selected from halo and (C₁-C₃)alkyl.

In some embodiments, the compound is a compound of Formula (I), wherein L¹ is C(R^{c})(R^{c*}) and one of R^{c} or R^{c*} and R* taken together with the atoms to which they are bonded form a 3- to 6-membered ring fused to Ring A; Z² is

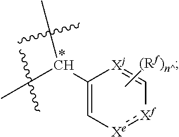

X^{h} is CH₂; X^{h*} is CH₂ or C(R^{e})₂; X^{g} is N; X^{e} is N; X^{f} is C(O) and the bond between X^{e} and X^{f} is a single bond; and R^{d*} is CH₃. In some embodiments, the 3- to 6-membered ring fused to Ring A forms a group having a structure selected from

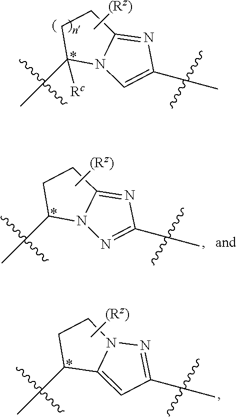

wherein R^{z} is absent or OH, n′ is 1 or 2, and the “*” at the ring carbon bonded to Ring B represents a third stereocenter. In some embodiments, the 3- to 6-membered ring fused to Ring A forms a group having a structure selected from

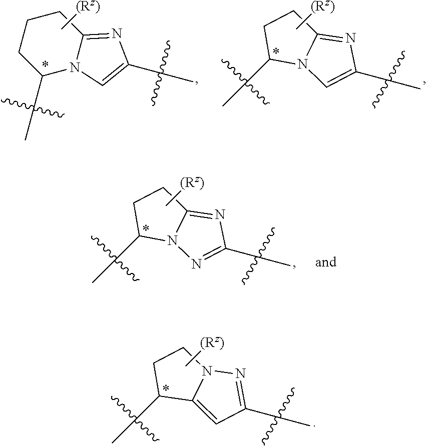

In some embodiments, R^{e} is F and n** is 2. In some embodiments, R^{f} is H. In some embodiments, R^{z} is absent and Ring B is phenyl or pyridinyl. In some embodiments, R^{d} is H; and each R is independently selected from halo and (C₁-C₃)alkyl.

In some embodiments, the compound of Formula (I) or a pharmaceutically acceptable salt thereof, is selected from compounds of Formula (Ia), (Ib), (Ic), (Id), and (Ie):

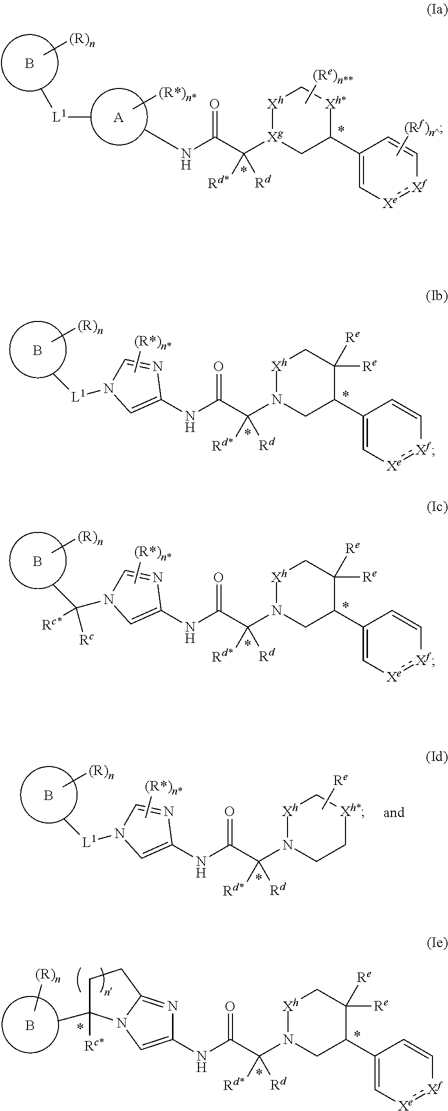

wherein n′ is 0, 1 or 2 and the “*” at the carbon bonded to both Ring B and R^{c*} represents and third stereocenter.

In some embodiments, the compound is a compound of Formula (Ie).

In some embodiments, the compound is a compound of Formula (Ia) or (Ib) or (Ic) and the first and second stereocenters are both in an S configuration.

In some embodiments, the compound is a compound of Formula (Id) and the first stereocenter is in an S configuration.

In some embodiments, the compound is a compound of Formula (Ie) and the first, and second stereocenters are in an S configuration. In some embodiments, the compound is a compound of Formula (Ie) and the first, and second stereocenters are in an S configuration and the third stereocenter is in an (R) configuration. In some embodiments, the compound is a compound of Formula (Ie) and the first, second, and third stereocenters are in an S configuration. In some embodiments, the compound is a compound of Formula (Id) and the first and second stereocenters are in an S configuration and the third stereocenter is in an R configuration.

In some embodiments, the compound is a compound of Formula (Ia) or (Ib) or (Ic) or (Id) and R* is (C₁-C₃)alkyl, halo, halo(C₁-C₃)alkyl or oxo. In some embodiments, R* is CH₃ or CF₃. In some embodiments, n* is 0 or 1.

In some embodiments, the compound is a compound of Formula (Ia) or (Ib) or (Ic) or (Id) or (Ie) and Ring B is phenyl or pyridinyl and each R is independently CN, SF₅, F, methyl, OCH₃, O-phenyl, or CF₃; and n is 1, 2 or 3.

In some embodiments, the compound is a compound of Formula (Ia) or (Id) and n** is 1 or 2.

In some embodiments, the compound is a compound of Formula (Ia) or (Ib) or (Ic) or (Id) or (Ie) and each R^{e} is independently halo, (C₁-C₃)alkyl, or (C₁-C₃)alkoxy. In some embodiments, each R^{e} is F. In some embodiments, each R^{e} together with the carbon to which they are bonded form a cyclopropyl group.

In some embodiments, the compound is a compound of Formula (Ia) or (Ib) or (Ic) or (Id) and L¹ is C(R^{c})(R^{c*}), O, or C(O). In some embodiments, each of R^{c} and R^{c*} is independently H, D, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halogen, (C₁-C₆)alkyl, halo(C₁-C₆)alkyl, and (C₃-C₈)cycloalkyl. In some embodiments, each of R^{c} and R^{c*} is independently H or CH₃, or R^{c} and R^{c*} together with the carbon to which they are bonded form a cyclopropyl group.

In some embodiments, the compound is a compound of Formula (Ia), (Ib), or (Id) and L is C(R^{c})(R^{c*}).

In some embodiments, the compound is a compound of Formula (Ia), (Ib), (Ic) or (Id) and one of R^{c} or R^{c*} and R* taken together with the atoms to which they are bonded form a 3- to 6-membered ring fused to Ring A. In some embodiments, R^{c} or R^{c*} and R* taken together with the atoms to which they are bonded fuse together with Ring A to form an 8 membered bicyclic heteroaryl or heterocyclic group having two nitrogen atoms. In some embodiments, 8 membered bicyclic heteroaryl or heterocyclic group having two nitrogen atoms comprises a 5 membered heteroaryl group fused to a 5 membered heterocyclic group.

In some embodiments, the compound is a compound of Formula (Ie) and n′ is 1 or 2. In some embodiments, n′ is 1. In some embodiments, n′ is 2. In some embodiments, R^{c*} is H or methyl. In some embodiments, Ring B is phenyl or pyridinyl and each R is independently CN, SF₅, F, Cl, methyl, OCH₃, O-phenyl, or CF₃; and n is 1, 2 or 3

In some embodiments, the compound is a compound of Formula (Ia) or (Ib) or (Ic) or (Ie) and X^{e} is NH and X^{f} is C═O and the bond between X^{e} and X^{f} is a single bond.

In some embodiments, the compound is a compound of Formula (Ia) or (Ib) or (Ic) or (Ie) and X^{e} is CH₂ and X^{f} is C═O and the bond between X^{e} and X^{f} is a single bond.

In some embodiments, the compound is a compound of Formula (Ia) or (Ib) or (Ic) or (Ie) and X^{e} is CH and X^{f} is N⁺—O⁻ and the bond between X^{e} and X^{f} is a double bond.

In some embodiments, the compound is a compound of Formula (Ia) or (Ib) or (Ic) or (Ie) and X^{e} is CH and X^{f} is N and the bond between X^{e} and X^{f} is a double bond.

In some embodiments, the compound is a compound of Formula (Ia) or (Ib) or (Ic) or (Ie) and X^{e} is N and X^{f} is C—CH₃, C—CH₂F, C—CHF₂. or CF₃ and the bond between X^{e} and X^{f} is a double bond.

In some embodiments, the compound is a compound of Formula (Ia) or (Id) and X^{h*} is CH₂, N and O. In some embodiments, the compound is a compound of Formula (Ia) or (Id) and X^{h*} is C(R^{e})₂ and R^{e} is F.

In some embodiments, the compound is a compound of Formula (Ia) or (Ib) or (Ic) or (Id) or (Ie) and X^{h} is CH₂, N and O.

In some embodiments, the compound is a compound of Formula (Ia) and ring A is imidazolylene, oxazolylene, oxadiazolylene, pyrazolylene, pyrrolylene, thiazolylene, or triazolylene. In some embodiments, Ring A is pyrrolylene, thiazolylene, imidazolylene, pyrazolylene, or triazolylene.

In some embodiments, the compound is a compound of Formula (Ia) and X^{g} is CH or N.

In some embodiments, the compound is a compound of Formula (Ia) or (Ib) or (Ic) or (Id) or (Ie) and each R^{d} and R^{d*} are independently H, (C₁-C₃)alkyl, or (C₁-C₃)alkoxy. In some embodiments, R^{d} and R^{d*} are the same and are H or (C₁-C₃)alkyl. In some embodiments, at least one of R^{d} and R^{d*} is (C₁-C₃)alkoxy. In some embodiments, each R^{d} and R^{d*} are independently chosen from H, CH₃, and OCH₃. In some embodiments, R^{d} and R^{d*} cyclize to form a cyclopropyl group.

In some embodiments, the compound is a compound of Formula (Ie), and Ring B is phenyl, n is 2, each R is independently CN, SF₅, F, methyl, OCH₃, O-phenyl, or CF₃, and each R^{e} is independently F, Me, or MeO.

In some embodiments, the compound of Formula (I) has the structure:

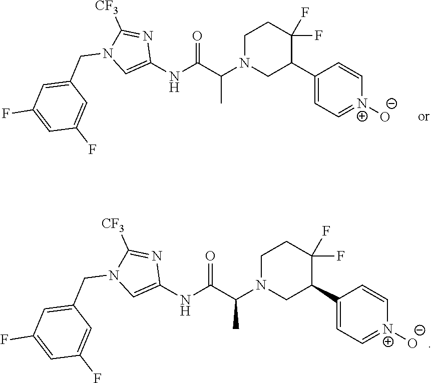 [SMILES: CC(C(=O)Nc1cn(Cc2cc(F)cc(F)c2)c(C(F)(F)F)n1)N1CCC(F)(F)C(c2cc[n+]([O-])cc2)C1 ; C[C@@H](C(=O)Nc1cn(Cc2cc(F)cc(F)c2)c(C(F)(F)F)n1)N1CCC(F)(F)[C@@H](c2cc[n+]([O-])cc2)C1]

pharmaceutically salt thereof.

In some embodiments, the compound of Formula (I) has the structure:

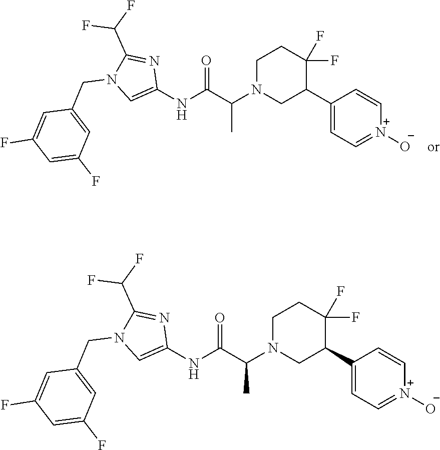 [SMILES: CC(C(=O)Nc1cn(Cc2cc(F)cc(F)c2)c(C(F)F)n1)N1CCC(F)(F)C(c2cc[n+]([O-])cc2)C1 ; C[C@@H](C(=O)Nc1cn(Cc2cc(F)cc(F)c2)c(C(F)F)n1)N1CCC(F)(F)[C@@H](c2cc[n+]([O-])cc2)C1]

pharmaceutically salt thereof.

In some embodiments, the compound of Formula (I) has the structure:

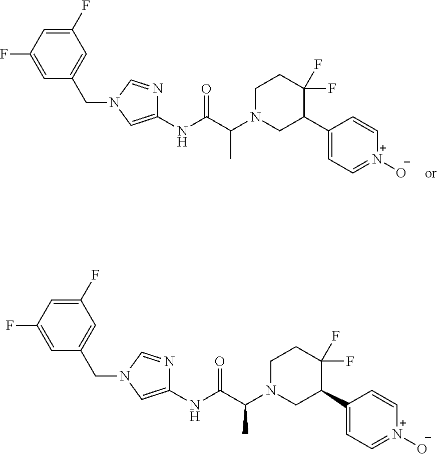 [SMILES: CC(C(=O)Nc1cn(Cc2cc(F)cc(F)c2)cn1)N1CCC(F)(F)C(c2cc[n+]([O-])cc2)C1 ; C[C@@H](C(=O)Nc1cn(Cc2cc(F)cc(F)c2)cn1)N1CCC(F)(F)[C@@H](c2cc[n+]([O-])cc2)C1]

pharmaceutically salt thereof.

In some embodiments, the compound of Formula (I) has the structure:

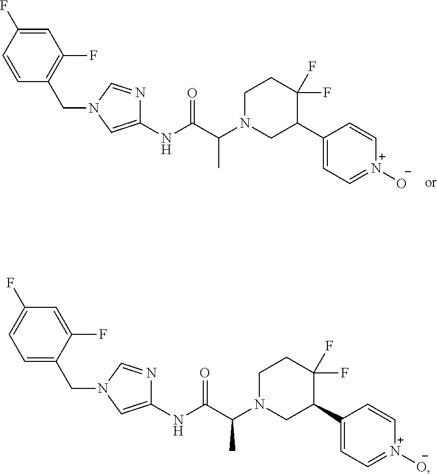

pharmaceutically salt thereof.

In some embodiments, the compound of Formula (I) is chosen from
4-(1-(1-((1-(2,4-difluorobenzyl)-1H-imidazol-4-yl)amino)-1-oxopropan-2-yl)-4,4-difluoropiperidin-3-yl)pyridine 1-oxide;
4-((S)-1-((S)-1-((1-(2,4-Difluorobenzyl)-1H-imidazol-4-yl)amino)-1-oxopropan-2-yl)-4,4-difluoropiperidin-3-yl)pyridine 1-oxide;
4-(1-(1-((1-(3,5-difluorobenzyl)-1H-imidazol-4-yl)amino)-1-oxopropan-2-yl)-4,4-difluoropiperidin-3-yl)pyridine 1-oxide;
4-((S)-1-((S)-1-((1-(3,5-difluorobenzyl)-1H-imidazol-4-yl)amino)-1-oxopropan-2-yl)-4,4-difluoropiperidin-3-yl)pyridine 1-oxide;
4-((S)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide; and
(S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide.

In some embodiments the compound of Formula I is chosen from:

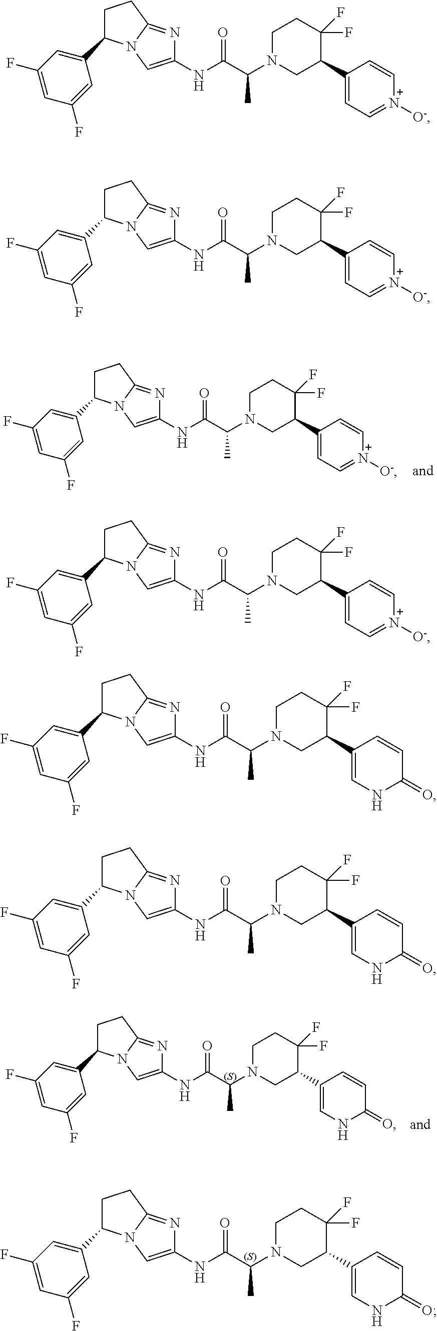

or a pharmaceutically acceptable salt thereof.

In some embodiments the compound of Formula I is a compound having the structure:

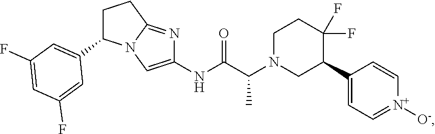

or a pharmaceutically acceptable salt thereof.

In some embodiments the compound of Formula I is a compound having the structure:

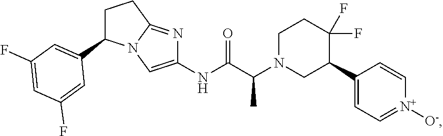

or a pharmaceutically acceptable salt thereof.

In some embodiments the compound of Formula I is a compound having the structure

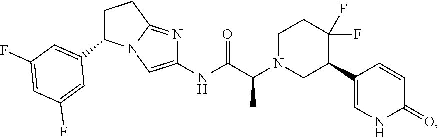 [SMILES: C[C@@H](C(=O)Nc1cn2c(n1)CC[C@H]2c1cc(F)cc(F)c1)N1CCC(F)(F)[C@@H](c2ccc(=O)[nH]c2)C1]

or a pharmaceutically acceptable salt thereof.

In some embodiments the compound of Formula I is a compound having the structure

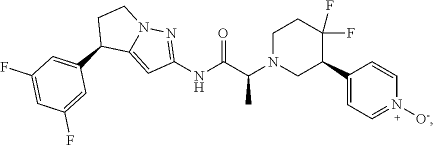

or a pharmaceutically acceptable salt thereof.

In some embodiments the compound of Formula I is a compound chosen from
2-(4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide;
(S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide;
(S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide;
(S)-2-((R)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide;
(S)-2-((R)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide;
4-(1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide; and
4-((S)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-(1-(1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((S)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((S)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((R)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((R)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
   or a pharmaceutically acceptable salt thereof.

In some embodiments, the compound of Formula (I) is a compound as recited in the Examples and Tables herein, or racemic mixtures thereof, or a pharmaceutically acceptable salt thereof.

In some embodiments, the compound of Formula (I) has at least one H replaced by deuterium, D. In some embodiments, R^{c} and/or R^{c*} are D.

### General Synthetic Methods

Certain processes for the manufacture of the compounds of this invention are provided as further features of the invention and are illustrated by the following exemplary reaction schemes. Those skilled in the art will appreciate that other synthetic routes may be used to synthesize the inventive compounds. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art. In particular, it is noted that the compounds prepared according to these Schemes may be modified further to provide new Examples within the scope of this invention. In addition, it will be evident from the detailed descriptions given in the Experimental section that the modes of preparation employed extend further than the general procedures described herein. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The starting materials are generally available from commercial sources such as Merck Sigma-Aldrich Inc. and Enamine Ltd. Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including Supplements (also available via the Beilstein online database).

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991 and Greene's Protective Groups inorganic Synthesis, John Wiley &Sons, New York 2006.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

More specifically intermediates and compounds of the present disclosure may be made by the methods shown in the following schemes.

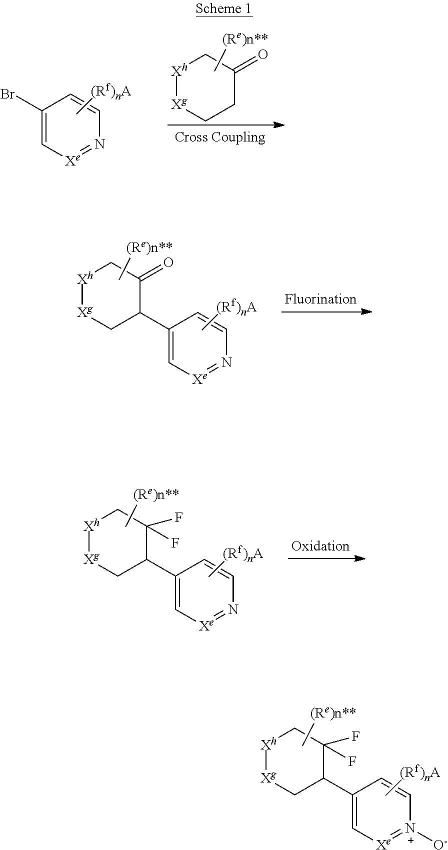

Step 1: Aryl bromides or any aromatic halogen compounds are coupled at the alpha position of a substituted ketone. Reaction conditions typically utilize polar aprotic solvents such as THF and strong bases such as sodium tert-butoxide in the presence of a ligated palladium catalyst, typically generated from palladium acetate with dicyclohexyl[2′,4′,6′-tris(propan-2-yl)[1,1′-biphenyl]-2-yl]phosphane or XPhos, under inert atmospheric conditions at elevated temperatures, typically 50° C., overnight. Step 2: Difluorination of the ketone is achieved with the N,N-diethyl-S,S-difluorosulfiliminium tetrafluoroborate or XtalFluor-E and triethylamine trihydrofluoride in polar aprotic solvents such as DCM. These transformations are typically run at 0° C. under inert atmospheric conditions overnight. Step 3: Oxidation of the pyridine can be achieved with oxidant such as potassium peroxymonosulfate or Oxone in a mixture of polar aprotic solvents with polar protic solvents in a 1:1 ratio. Typical solvents that are employed in this reaction are acetone and water. Step 4: If required, Boc-deprotection of X^{g} can be achieved using an acid such as 4M HCl in dioxane at room temperature overnight.

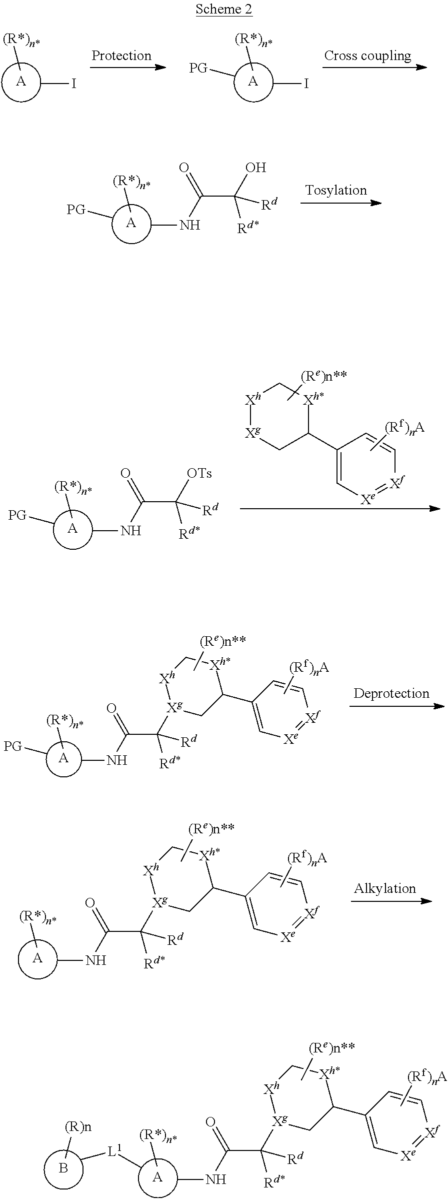

Step 1: Protection of a halo substituted nitrogen containing heteroaromatic can be achieved with triphenylmethyl chloride and triethylamine at 0° C. for two hours in polar aprotic solvents. Of particular interest are imidazoles with iodo substitution. Step 2: A substituted amide, can be coupled to a substituted heteroaromatic halide using a copper source such as copper iodide, a base such as potassium carbonate, and a ligand such as (1S,2S)—N,N′-dimethylcyclohexane-1,2-diamine, L-proline, or 1,2-bis(dimethylamino)ethane, in a polar aprotic solvent such as 1,4-dioxane and heated at elevated temperatures around 80° C. to 110° C. for 2-24 h. Step 3: Activation of the alcohol can be achieved using tosyl-Cl and pyridine or triethylamine in polar aprotic solvents or in neat pyridine overnight. Step 4: Nucleophilic displacement of the toyslate group can be achieved with amines in polar aprotic solvents with bases. Step 5: Deprotection of the heterocycles can typically be achieved with an acid source such as HCL in 2M dioxane. Step 6: Alkylation of heterocycles can be achieved with benzyl halides or any other electrophiles under basic conditions in polar aprotic solvents.

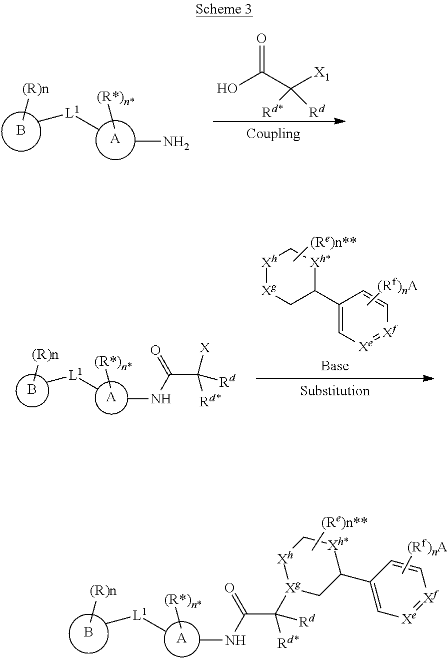

Step 1: Coupling of a substituted acid with a substituted amino heterocycle can be accomplished using standard peptide coupling conditions. These conditions may include but are not limited to the use of a coupling reagent such as DCC and additive such as silver nitrate or DMAP in a polar aprotic solvent such as THF or DCM with stirring overnight at RT. Step 2: Nucleophilic displacement of the halide group (X₁) can be achieved with amines in polar aprotic solvents with bases.

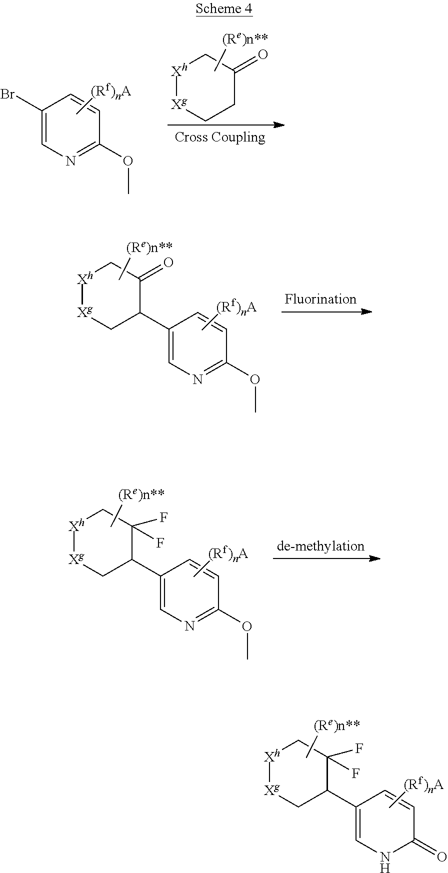

Step 1: Aryl bromides or any aromatic halogen compounds, in this case a substituted methoxypyridine, are coupled at the alpha position of a substituted ketone. Reaction conditions utilize the polar aprotic solvents such as THF and strong bases such as sodium tert-butoxide in the presence of a ligated palladium catalyst, typically generated from palladium acetate with dicyclohexyl[2′,4′,6′-tris(propan-2-yl)[1,1′-biphenyl]-2-yl]phosphane or XPhos, under inert atmospheric conditions at elevated temperatures, typically 50° C., overnight. Step 2: Difluorination of the ketone can be achieved with N,N-diethyl-S,S-difluorosulfiliminium tetrafluoroborate or XtalFluor-E and triethylamine trihydrofluoride in polar aprotic solvents such as DCM. These transformations are typically run at 0° C. under inert atmospheric conditions overnight. Step 3: De-methylation of the methoxy group can be accomplished by using HBr/AcOH with heating at elevated temperatures, typically around 100° C. for several hours. Step 4 (If required): Boc-deprotection of X^{g} can be achieved with an acid such as 4M HCl in dioxane at room temperature overnight.

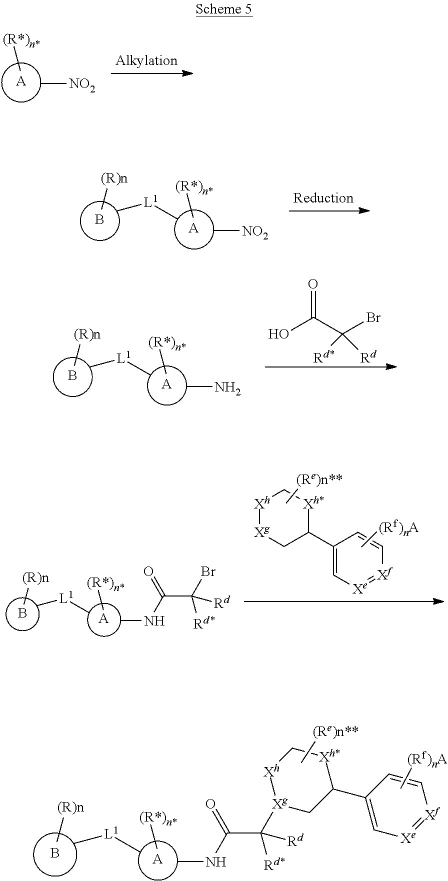

Step 1: Substituted heteroaromatics (where Z₁ is NH) can be alkylated with an electrophile, typically a benzylic halide, using a base such as potassium carbonate, in a polar aprotic solvent such as acetonitrile, with heating at elevated temperatures. Step 2: Substituted heteroaromatic nitro groups can be reduced to their corresponding amino groups with tetrahydroxydiboron and 4, 4′-bypyridine in DMF at 0° C.→RT. Step 3: Coupling of a substituted acid with a substituted amino heterocycle can be accomplished using standard peptide coupling conditions. These conditions may include but are not limited to the use of a coupling reagent such as DCC and additive such as silver nitrate or DMAP in a polar aprotic solvent such as THF or DCM with stirring overnight at RT. Step 4: Nucleophilic displacement of the alkyl bromide can be achieved with amines in polar aprotic solvents with bases.

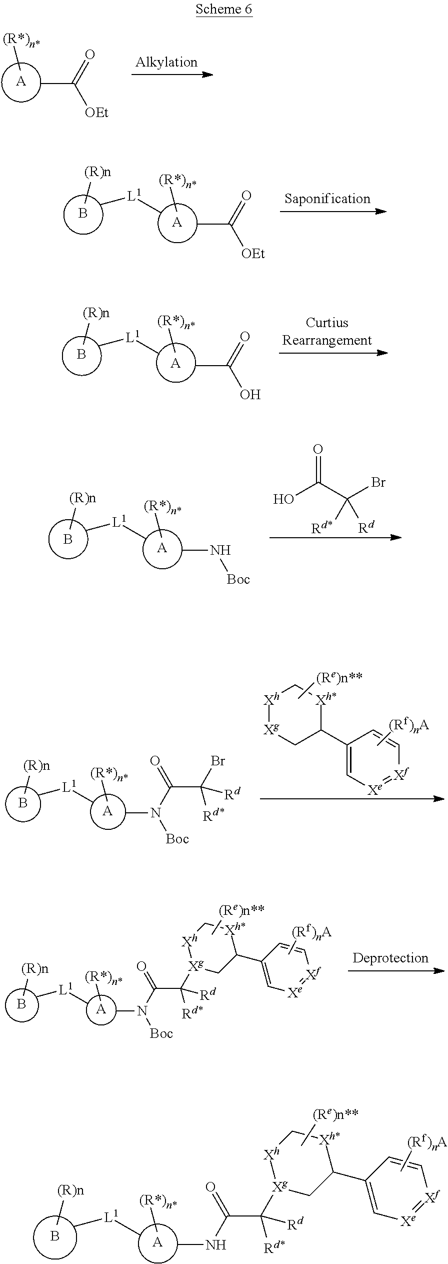

Step 1: Substituted heteroaromatics, typically substituted with an ester and in the case where Z₁ is CH, can be coupled with a halogen containing compound, typically a benzylic halide, using a transition metal catalyst such as palladium acetate, a ligand such as CyJohnPhos, and a base such as cesium carbonate, in a polar aprotic solvent such as 1,4-dioxane, and heated at elevated temperatures, typically 100° C., for several hours. Step 2: Saponification of a substituted heteroaromatic ester can be accomplished using a strong base such as lithium hydroxide, in a mixture of THF and water at room temperature or with heating. Step 3: Substituted heteroaromatic carboxylic acids can undergo a Curtis rearrangement to generate protected amines, in this case a Boc protected amine, by using DPPA and triethylamine in tert-butanol with heating from 0° C.->100° C. Step 4: Coupling of a substituted acid with a substituted amino heterocycle can be accomplished using standard peptide coupling conditions. These conditions may include but are not limited to the use of a coupling reagent such as DCC and additive such as silver nitrate or DMAP in a polar aprotic solvent such as THF or DCM with stirring overnight at RT. Step 5: Nucleophilic displacement of the alkyl bromide can be achieved with amines in polar aprotic solvents with bases. Step 6: Boc-deprotection can be achieved with an acid such as 4M HCl in dioxane at room temperature overnight.

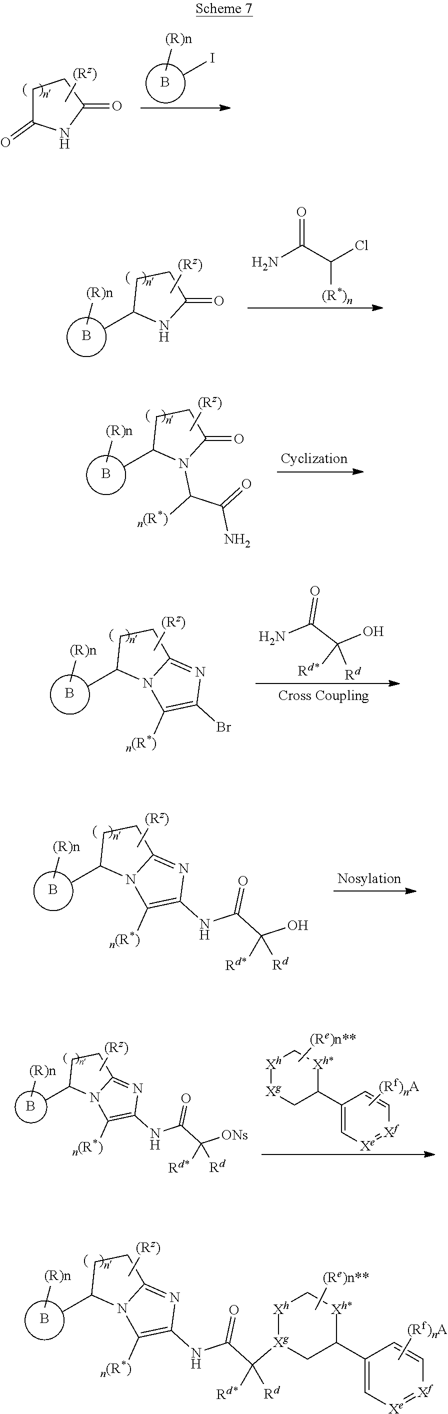

Step 1: A nucleophile, typically one that can be generated from the addition of isopropyl magnesium bromide with a substituted aromatic halide in a polar aprotic solvent such as THF at −30° C.→RT, can be added to succinimide at −78° C.→RT. The resulting adduct can then be reduced with sodium cyanoborohydride at temperatures that range from 0° C.→RT to provide substituted pyrrolidinones. Step 2: Alkylation of a pyrrolidinone can be accomplished using a base such as sodium hydride, in a polar aprotic solvent such as THF. Step 3: Cyclization can be achieved using phosphoryl bromide in a polar aprotic solvent such as acetonitrile with heating at elevated temperatures, typically 80° C. after several hours to afford the corresponding substituted 5,5-fused heteroaromatic halide. Step 4: A substituted amide, can be coupled to a substituted heteroaromatic halide using a copper source such as copper iodide, a base such as potassium carbonate or potassium phosphate tribasic, and a ligand such as (1S,2S)—N,N′-dimethylcyclohexane-1,2-diamine, L-proline, or 1,2-bis(dimethylamino)ethane in a polar aprotic solvent such as dioxane and heated at elevated temperatures around 80° C. to 110° C. for 2-24 h. Step 5: Activation of the alcohol can be achieved with NsCl (2-nitrobenzenesulfonyl chloride), DMAP and triethylamine in polar aprotic solvents such as dichloromethane at 0° C. after several hours. Step 6: Nucleophilic displacement of the nosylate group can be achieved with amines in polar aprotic solvents with bases.

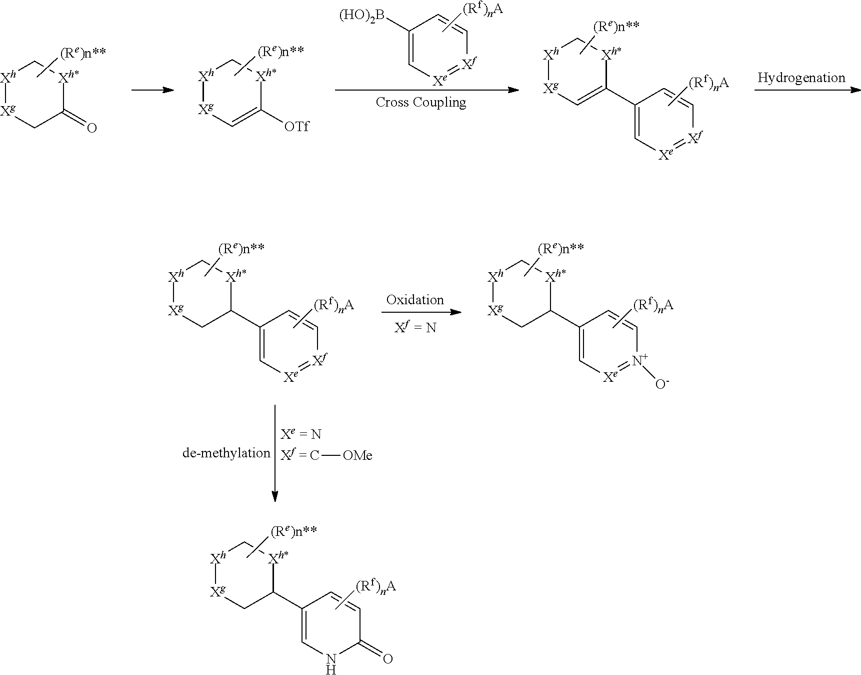

Step 1: Enol triflates can be formed by reacting an enolizable carbonyl containing compound with base, such as LiHMDS, and a triflating reagent, such as phenyl triflimide, in a polar aprotic solvent, such as THF, typically at temperatures that range from −78° C. to RT for 1-24 h. Step 2: Coupling of an enol triflates with a boronic acid can be accomplished by using a palladium source, such as Pd(dppf)Cl₂, a base, such as cesium carbonate, in a mixture of a polar aprotic solvent, such as 1,4-dioxane or THF, and water, at elevated temperatures, typically 40° C. to 100° C., under inert atmospheric conditions overnight. Step 3: Reduction of alkenes can typically be accomplished with a palladium catalyst, such as palladium on charcoal, in a polar protic solvent, such as methanol, under an atmosphere of hydrogen at room temperature over the course of 1-24 h. Step 4-1 (optional in certain cases where X^{f}═N): Oxidation can be achieved with oxidant such as potassium peroxymonosulfate or Oxone in a mixture of polar aprotic solvents with polar protic solvents in a 1:1 ratio. Typical solvents that are employed in this reaction are acetone and water. Step 4-2 (optional in certain cases where X^{e}═N and X^{f}=C—OMe): De-methylation of the methoxy group can be accomplished by using HBr/AcOH with heating at elevated temperatures, typically around 100° C. for several hours.

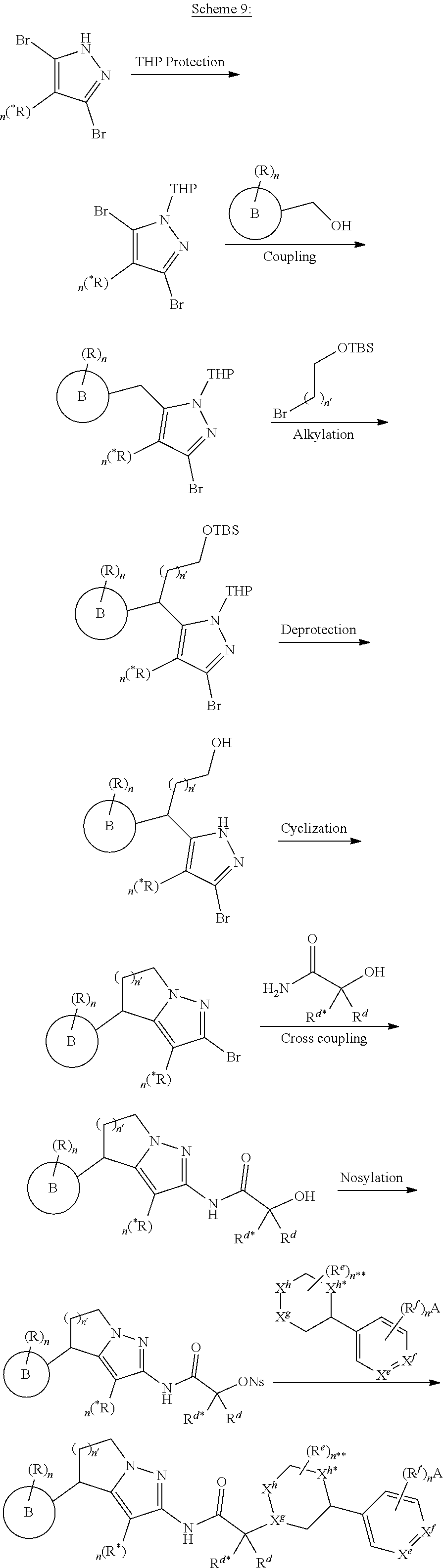

Step 1: THP protection of pyrazoles can be accomplished with 3,4-dihydro-2H-pyran and an acid catalyst, such as TsOH, in a polar aprotic solvent, such as THF, at temperatures that range from RT to 70° C., over 1-24 h. Step 2: Coupling of a halogenated pyrazole with an alcohol can be achieved using NHC, such as 5,7-di-tert-butyl-3-phenylbenzo[d]oxazol-3-ium tetrafluoroborate, an iridium catalyst, such as [Ir(ppy)₂(dtbbpy)]PF₆, a nickel catalyst, such as [4,4′-Bis(tert-butyl)-2,2′-bipyridine]nickel dibromide, and base, such as 1-azabicyclo[2.2.2]octane, in mixtures of pyridine, DMAC and MTBE with photo irradiation with LEDs, typically at 450 nm, under an atmosphere of nitrogen overnight. Step 3: Alkylation of pyrazoles with electrophiles can be accomplished by using a strong base, such as LDA, in a polar aprotic solvent, such as THF, at temperatures that range from −78° C. to 0° C., for 1-24 h under an inert atmosphere. Step 4: Deprotection of acid labile protecting groups can be accomplished with an acid, such as HCl in 1,4-dioxane, at RT for 1-24 h. Step 5: Fused bicyclic pyrazoles can be formed via cyclization under Mitsunobu conditions that typically utilize triphenylphospine and DEAD or DIAD in polar aprotic solvents such as THF, at temperatures that range from 0° C. to 60° C. under an inert atmosphere over 1-24 h. Step 6: A substituted amide, can be coupled to a substituted heteroaromatic halide using a copper source such as copper iodide, a base such as potassium carbonate or potassium phosphate tribasic, and a ligand such as (1S,2S)—N,N′-dimethylcyclohexane-1,2-diamine, L-proline, or 1,2-bis(dimethylamino)ethane in a polar aprotic solvent such as dioxane and heated at elevated temperatures around 80° C. to 110° C. for 2-24 h. Step 7: Activation of the alcohol can be achieved with NsCl (2-nitrobenzenesulfonyl chloride), DMAP and triethylamine in polar aprotic solvents such as dichloromethane at 0° C. after several hours. Step 8: Nucleophilic displacement of the nosylate group can be achieved with amines in polar aprotic solvents with bases.

As can be appreciated by the skilled artisan, the above synthetic Schemes and representative examples (below) are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### Methods of Treating, Administration, and Pharmaceutical Compositions

In one aspect, provided herein are methods of modulating MRGPRX2 activity comprising administering an effective amount of the compound of Formula (I) and/or a pharmaceutically acceptable salt thereof, or a pharmaceutical composition as described herein.

Also provided, in some embodiments, is a method of modulating mast cell degranulation comprising administering an effective amount of the compound of Formula (I) and/or a pharmaceutically acceptable salt thereof, or a pharmaceutical composition as described herein.

Also provided, in some embodiments, is a method of treating, preventing or ameliorating an MRGPRX2-mediated disease or disorder in a subject in need thereof comprising administering to the subject an effective amount of the compound of Formula (I) and/or a pharmaceutically acceptable salt thereof, or a pharmaceutical composition as described herein. In some embodiments, the disease is chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain.

Also provided, in some embodiments, is a method of treating a condition chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain comprising administering to the subject an effective amount of the compound of Formula (I) and/or a pharmaceutically acceptable salt thereof, or a pharmaceutical composition as described herein.

In some embodiments, the condition is atopic dermatitis.

Also provided, in some embodiments, is the compound of Formula (I) and/or a pharmaceutically acceptable salt thereof for use as a medicament. In some embodiments, the compound is for use in treating disease or disorder mediated by MRGPRX2.

Also provided, in some embodiments, is the use of the compound of Formula (I) and/or a pharmaceutically acceptable salt thereof in the manufacture of a medicament for treating a MRGPRX2-mediated disease or disorder.

### Pharmaceutical Compositions and Formulations

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. In one embodiment, the dosage level will be about 0.1 to about 250 mg/kg per day. In another embodiment the dosage level will be about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

As the compounds of this invention may have utility for the treatment of atopic dermatitis the compounds may be administered topically. Thus, compounds of the invention may be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

Also provided, in some embodiments, is a pharmaceutical composition comprising the compound of Formula (I) and/or a pharmaceutically acceptable salt thereof and a pharmaceutically acceptable excipient.

Pharmaceutical compositions can be formulated using one or more pharmaceutically acceptable carriers comprising excipients and auxiliaries. The formulation can be modified depending upon the route of administration chosen. The pharmaceutical compositions can also include the compounds described herein in a free base form or a pharmaceutically acceptable salt form.

Methods for formulation of the pharmaceutical compositions can include formulating any of the compounds described herein with one or more inert, pharmaceutically acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

The pharmaceutical compositions provided herein may be formulated as immediate or modified release dosage forms, including delayed-, sustained, pulsed-, controlled, targeted-, and programmed-release forms. Thus, in some preferred embodiments, the active ingredient(s) is administered in a pharmaceutical composition which is an immediate release oral dosage form, preferably but not necessarily including an enteric coating. In some preferred embodiments, the active ingredients(s) are administered in a pharmaceutical composition which is an extended-release oral dosage form, preferably but not necessarily including an enteric coating. In further preferred embodiments, the active ingredients are administered in a pharmaceutical composition which contains both an immediate release dose and an extended-release dose or pulsed release dose of the first agent preferably but not necessarily also including an enteric coating. Such dual release dosage forms achieve release of an initial dose of active ingredient, followed late in time by another pulsed release, or by a sustained release dose. Methodologies for preparing such dual release dosage forms are well known in the art.

In some embodiments, the active ingredients are formulated into a controlled release matrix tablet, which contains one or more polymeric matrix materials that promote the sustained, delayed or pulsed release profile. Non-limiting examples of such polymeric matrix materials include cellulosic materials as described above, and carbomers, for example those sold by Lubrizol Corporation under the name Carbopol®, for example Carbopol® 71G NF, Carbopol® 971P NF and Carbopol® 974P NF polymers.

Some preferred examples of extended-release compositions suitable for use in the methods and compositions of the invention include, for example and not limitation, extended-release compositions found in nifedipine formulations such as Adalat CC®, Procardia® XL, Afeditab® CR and Nifedical® XL; and in diltiazem formulations such as Cardizem® CD, Cardizem® LA, Cardizem® SR, Cartia® XT and Dilacor® XR.

Pharmaceutical formulations of the compositions described herein can be prepared for storage by mixing a compound with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together are safer or more effective than either drug alone. In some embodiments the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form.

Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. In some embodiments, the combination agent is a drug for reduction of symptoms of ALS. In some embodiments, the combination agent is selected from an NAD supplement (such as nicotinamide riboside, offered under the trade names Basis® or Tru Niagen®), vitamin B₁₂ (oral or injection), glycopyrrolate, atropine, scopolamine, baclofen, tizanidine, mexiletine, an SSRI, a benzodiazepine, Neudexta, riluzole, and edaravone, and combinations thereof.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of Such compound or prodrug and a second compound as described above. The kit comprises a means for containing the separate compositions such as a container, a divided bottle or a divided foil packet. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. Second Week, Monday, Tuesday, . . . etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this. In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered microchip memory coupled with a liquid crystal readout, or audible reminder signal which, for example, reads out the date that the last daily dose has been taken and/or reminds one when the next dose is to be taken. Also, as the present invention has an aspect that relates to the treatment of the disease/conditions described herein with a combination of active ingredients which may be administered jointly, the invention also relates to combining separate pharmaceutical compositions in a single dosage form, Such as (but not limited to) a single tablet or capsule, a bilayer or multilayer tablet or capsule, or through the use of segregated components or compartments within a tablet or capsule. The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human.

The present invention further comprises use of a compound of Formula I for use as a medicament (Such as a unit dosage tablet or unit dosage capsule). In another embodiment, the present invention comprises the use of a compound of Formula I for the manufacture of a medicament (such as a unit dosage tablet or unit dosage capsule) to treat one or more of the conditions discussed herein.

In practicing the methods described herein, therapeutically effective amounts of the compounds or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically effective amount can vary widely depending on the severity of the disease, the age and relative health of the subject, the potency of the compounds used, and other factors.

Prevent, preventing, and the like can refer to the prevention of the disease or condition in the patient. For example, if an individual at risk of contracting a disease is treated with the methods of the present disclosure and does not later contract the disease, then the disease has been prevented, at least over a period of time, in that individual.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

Natural endogenous ligands of Mrgprb2/MRGPRX2 have been reported and are mostly neuropeptides, including substance P (SP), vasoactive intestinal polypeptide (VIP), Cortistatin-14, and pituitary adenylate cyclase activating polypeptide (PACAP). Others include B-defensin, cathelicidin (LL-37), and proadrenomedullin N-terminal 20 peptide (PAMP9-20) (Journal of Allergy and Clinical Immunology, 138, 700-710 (2016); J. Immunol., 191, 345-352 (2013); BBRC, 349, 1322-28 (2006)). Given the close proximity between mast cells and sensory nerves in various pathological conditions, it follows that neuropeptide-activated MRGPRX2 could contribute to symptoms of neurogenic inflammation including pain, swelling and pruritus. Various observations using knock-out (KO) mice are consistent with the Mrgprb2/MRGPRX2 receptors playing a role in mast cell-mediated neurogenic inflammation. For instance, Mrgprb2/MRGPRX2 agonists induce various symptoms such as flushing, swelling and itch in wild type mice, but not in Mrgprb2-deficient mice (Nature, 519, 237-241 (2015); Immunity, 50, 1163-1171 (2019)). Mrgprb2-deficient mice have also demonstrated significant reductions in inflammation (leukocyte infiltration, including mast cells), swelling, pain and overall clinical score in various disease models (Neuron, 101, 412-420 (2019); Immunity, 50, 1163-1171 (2019); Nature Immunology, 20, 1435-1443 (2019)). An important and relevant observation was the demonstration that Substance P injection could stimulate the infiltration of leukocytes in wild type and NKRl (canonical Substance Preceptor) KO mice whereas the response was substantially blunted in Mrgprb2 null mice (Neuron, 101, 412-420 (2019)). This observation extends the role of Mrgprb2/MRGPRX2 as a key receptor in mediating Substance P-induced inflammatory responses, including pain (Neuron, 101, 353-355, (2019)). Indeed, a Substance P/Mrgprb2 sensory cluster was demonstrated to be critical in driving the clinical score of a severe preclinical model of atopic dermatitis (Nature Immunology, 20, 1435-1443 (2019)).

In addition to the various reports using Mrgprb2-deficient mice, further evidence suggests a role for various ligands of MRGPRX2 in human disease. For example, in addition to the number of MRGPRX2-expressing mast cells being significantly increased in severe chronic urticaria (Clinical and Molecular Allergy 16, 24 (2018)), PACAP nerve fibers were demonstrated to be in close proximity to tryptase-positive mast cells, providing the morphological basis for increased mast cell—sensory interactions (J. Allergy Clin. Immunol., 134, 622-633 (2014)). In support of this, patients with urticaria exhibit enhanced wheal reactions vs healthy individuals to MRGPRX2 agonists (e.g., Substance P) when injected intradermally (Allergy, 54, 46-56 (1999)). In addition, PACAP and the antimicrobial peptide, LL-37, which is implicated in cutaneous inflammation, were both demonstrated to be upregulated in rosacea (J. Invest. Dermatol., 15, 53-62, (2011)). Indeed, mast cell-deficient mice do not develop inflammation/flushing following L.L-37 injection (J. Inv. Derm., 134, 2728 (2014)) thus inferring a role for Mrgprb2.

In addition to skin disorders, mast cell involvement has been highlighted for inflammatory bowel disease (IBD) and arthritis (Immunol. Rev., 217, 38-52 (2007); Pharmacology and Therapeutics, 116, 207-235 (2007)) as well as asthma (Respiratory Research, 19, 1 (2018)) and migraine. In patients with rheumatoid arthritis (RA), the number of degranulated mast cells is increased in synovial tissue and is correlated with disease activity, as it is for patients with IBD. A positive correlation between serum Substance P levels and chronic pain intensity has been noted in both osteoarthritic and RA patients (PLOS ONE, 10, e0139206 ((2015)) and a recent article suggested that the SP-MRGPRX2 axis may play a role in the pathogenesis of RA, especially in the regulation of inflammation and pain (Allerg. Intern., 26, S9-S20 (2017)). Finally, there is a growing body of evidence for a role of PACAP in migraine pathogenesis and that it is mediated via activation of mast cells (Frontiers in Cellular Neuroscience, 13, 1-11 (2019)).

The Formula I compounds of this invention, their prodrugs and the salts of such compounds and prodrugs are all adapted to therapeutic use as agents that mediate the Mas-related G protein-coupled receptor (MRGPRX2) in mammals, particularly humans. For example, these compounds act as MRGPRX2 receptor antagonists and thus are useful for the treatment of the various conditions (e.g., those described herein) in which such action is implicated.

Given the positive correlation between activation of the MRGPRX2 receptor with the development of itch associated conditions, a pain associated condition, a pseudo-allergic reaction, an autoimmune or inflammatory disorder, or cancer-associated condition, Formula I compounds of this invention, their prodrugs and the salts of such compounds and prodrugs, by virtue of their pharmacologic action, are useful for the prevention, arrestment and/or regression of an itch associated condition, a pain associated condition, a pseudo-allergic reaction, an autoimmune or inflammatory disorder, or cancer-associated condition.

As used herein the phrase “pseudo-allergic reaction” refers to an IgE-independent allergic reaction, characterized by histamine release, inflammation, airway contraction, or any combination thereof. A pseudo-allergic reaction may be an anaphylactic reaction. A pseudo-allergic reaction may be caused by a range of cationic substances, collectively called basic secretagogues, including inflammatory peptides and drugs associated with allergic-type reactions. Thus, in one embodiment, the method of present invention is provided to treat a pseudo-allergic reaction, such as pseudo-allergic reactions caused by secretagogues, cationic peptidergic drugs, anionic peptidergic drugs, neutral peptidergic drugs, non-steroidal antagonist drugs, neuropeptides, and antimicrobial peptides. In one embodiment, the pseudo-allergic reaction is caused by MCD peptide, Substance P, VIP, PACAP, dynorphin, somatostatin, Compound 48/80, cortistatin-14, mastoparan, melettin, cathelicidin peptides, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, and rocuronium.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; contact dermatitis; allergic blepharitis; anaphylaxis; anaphylactoid drug reactions; Anaphylactic shock; Anemia; Atopic dermatitis; Bullous pemphigoid; Candidiasis; Chicken pox; end-stage renal failure; hemodialysis; Cholestatic pruritis; Chronic urticaria; Contact dermatitis, Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyria; Lymphoma; Mast cell activation syndrome, Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudo-allergic reactions; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Uremic Pruritus; Rosacea; Cutaneous amyloidosis; Scleroderma; Acne; wound healing; burn healing; ocular itch; and Urticaria.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoidcell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, swannamatoisis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, Biliary atresia, acute and chronic Hepatitis Band C virus, Liver tumors and cancer, Alcoholic liver disease, Polycystic liver disease, Liver cholangiocarcinoma, neuromyelitis optica spectum disorder, cardiovascular disease, and vasculitis.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Corneal Neuropathic Pain, Crohn's Disease, Degenerative Disc Disease, Dental Pain, Dercum's Disease, Dermatomyositis, Diabetic Peripheral Neuropathy (DPN), Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorrhaphy Pain Syndrome, Post Mastectomy, Postoperative Pain, Pain Syndrome, Post Stroke Pain, Post Thoracotomy Pain Syndrome, Postherpetic Neuralgia (Shingles), Post-Polio Syndrome, Primary Lateral Sclerosis, Psoriatic Arthritis, Pudenda! Neuralgia, Radiculopathy, Raynaud's Disease, Rheumatoid Arthritis (RA), Sacroiliac Joint Dysfunction, Sarcoidosis, Scheuermann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain and Vulvodynia.

As used herein the phrase “cancer associated condition” means any disease arising from the proliferation of malignant cancerous cells. Thus, in one embodiment, the method of present invention is provided to treat a cancer/tumor associated condition, such as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, birt-hogg-dube syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), Liver Cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomastosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Werner syndrome and Xeroderma pigmentosum.

### EXAMPLES

The following preparations of compounds of Formula (I) and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) and sub formulae and species described herein, including those where the substituent groups as defined herein, can be prepared as illustrated and described below.

Unless otherwise noted, all reagents were used without further purification. ¹H NMR spectra were obtained in CDCl₃, DMSO-d₆, or CD₃OD, unless stated otherwise, at room temperature on a Bruker AVANCE III HD 300 MHz, Bruker AVANCE III HD 400 MHz, or AVANCE NEO 400 MHz instrument or an NMR spectrometer of similar caliber. When more than one conformer was detected, the chemical shifts for the most abundant one is reported. Chemical shifts of ¹H NMR spectra were recorded in parts per million (ppm) on the 6 scale from an internal standard of residual solvent. Splitting patterns are designed as s, singlet; d, doublet; t, triplet; q, quartet; m, multiplet; br, broad.

The following abbreviations are used in the text: LCMS=liquid chromatography-mass spectrometry, ESI=Electrospray Ionization, M+H=a unit higher than the monoisotopic mass of the uncharged molecule, HPLC=High pressure liquid chromatography, Prep-HPLC=preparatory scale HPLC, AcOH=acetic acid, AIBN=azobisisobutyronitrile, ACN or MeCN=acetonitrile, PE=petroleum ether, EA or EtOAc=ethyl acetate, XtalFluor-E=N,N-Diethyl-S,S-difluorosulfiliminium tetrafluoroborate, BSA=Bovine Serum Albumin, DAST=Diethylaminosulfur trifluoride, DIEA=N,N-diisopropylethylamine, DEA=diethanolamine, DMA=Dimethylacetamide, DMAP=4-dimethylaminopyridine, DMEDA=N,N′-Dimethylethylenediamine, DMSO=dimethyl sulfoxide, DMF=N, N-dimethylacetamide, DCC=N,N′-Dicyclohexylcarbodiimide, DCM=dichloromethane, DMEM=Dulbecco's Modified Eagle Medium, DPPA=Diphenylphosphoryl azide, DIBAL=Diisobutylaluminium hydride, EtOH=ethanol, EGTA=(ethylene glycol-bis(3-aminoethyl ether)-N,N,N′,N′-tetraacetic acid), FA=formic acid, MeOH=methanol, MtBE=methyl tert-butyl ether, MCPBA=meta-Chloroperoxybenzoic acid, NBS=n-bromosuccinimide, NCS=n-chrlorosuccinimide, Otf=Trifluoromethanesulfonate, TEA or Et₃N=triethylamine, GOI=genes of interest, TsCl=p-toluenesulfonyl chloride, hERG=human ether-a-go-go-related gene, HBSS=Hanks' Balanced Salt Solution, HEPES=4-(2-hydroxyethyl)-1-piperazineethane sulfonic acid, HOBT=Hydroxybenzotriazole, LDA=Lithium diisopropylamide, SFC=Supercritical fluid chromatography, THF=tetrahydrofuran, TLC=thin layer chromatography, TBAF=tetra-n-butylammonium fluoride, TMSBr=Bromotrimethylsilane, TMSCl=trimethylsilyl chloride, TMSCN=trimethylsilyl cyanide, TCFH=Chloro-N,N,N′,N′-tetramethylformamidinium Hexafluorophosphate, TrtCl=Triphenylmethyl chloride, t-BuOH=tert-butyl alcohol, t-BuONa=sodium tert-butyloxide, Oac=acetate, NMI=1-Methylimidazole, POCl₃=Phosphoryl chloride, POBr₃=Phosphoryl chloride, py=pyridine, Pd/C=palladium on carbon, HTRF—Homogeneous Time-Resolved Fluorescence, RT=retention time, h=hour, hrs=hours, aq.=aqueous, min.=minute, sat.=saturated, equiv=equivalent, e.e.=enantiomeric excess, UV=ultraviolet, XPhos=2-Dicyclohexylphosphino-2′,4′,6′-triisopropylbiphenyl, Ns=2-nitrobenzene-1-sulfonyl.

Unless otherwise indicated, the chemical names used herein are generated using the Chemdraw Professional Version 23.1.1 64-bit, or Chemdraw Professional Version 16.0, or Marvin JS 21.19.0 software naming programs.

### Synthesis and Characterization Examples

For certain compounds disclosed herein the absolute stereochemistry has not been independently confirmed. At least one or more of the stereocenters' absolute stereochemistry are believed to be accurately represented in the name and structure of the disclosed compounds. For some compounds the stereochemistry is assigned based on analogy to known literature stereoisomers. For example, Intermediate B was determined to be the preferred stereoisomer for potency and was assigned (S) based on prep chiral HPLC and analogy to known literature stereoisomers. See WO2022/073904 Example 132 for assignment of stereochemistry for Intermediate B. Inspection of VCD data in the analysis range indicated that the VCD spectrum for fs1ss is a good match with experimental determined values. The stereochemistry of the substituted methylene moiety (—C(R^{d*})(R^{d})—) is believed to be accurate based on the absolute stereochemical assignment from commercially available vendors. (R)-2-hydroxypropanamide, also referred to as (R)-(+)-Lactamide, was either purchased from Bide Pharmatech Ltd. (CAS #598-81-2; Catalog #BD47136) or BBChem Co., Ltd (CAS #598-81-2; Catalog #BB211065; Batch #BB211065A23082301).

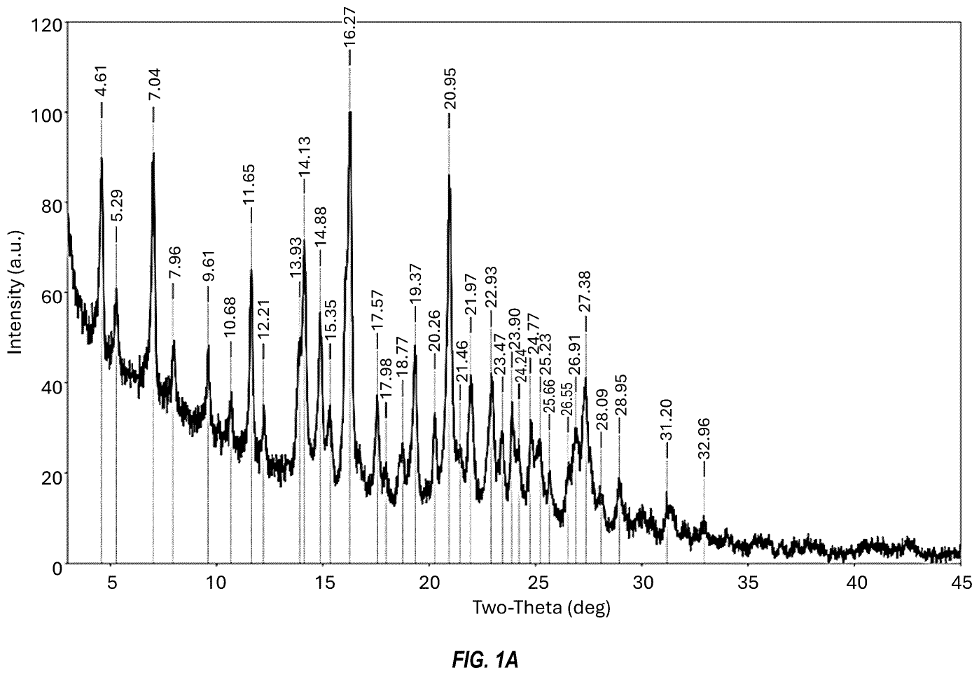

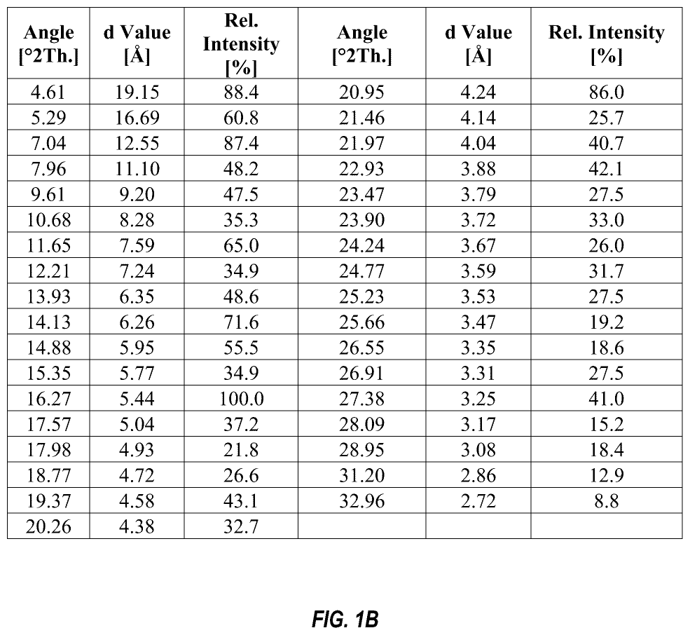

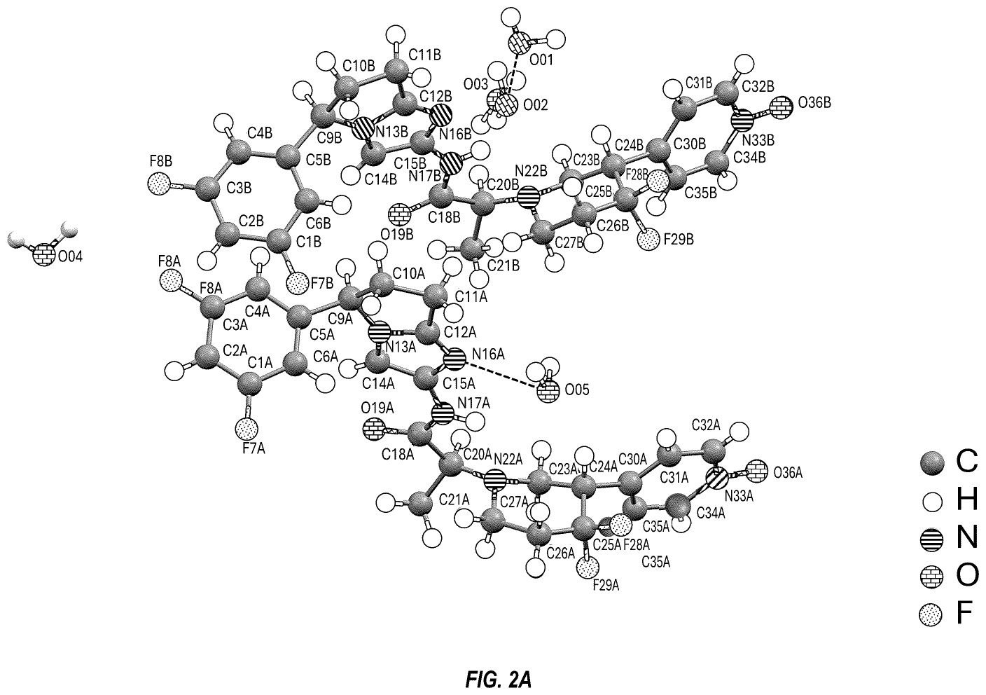

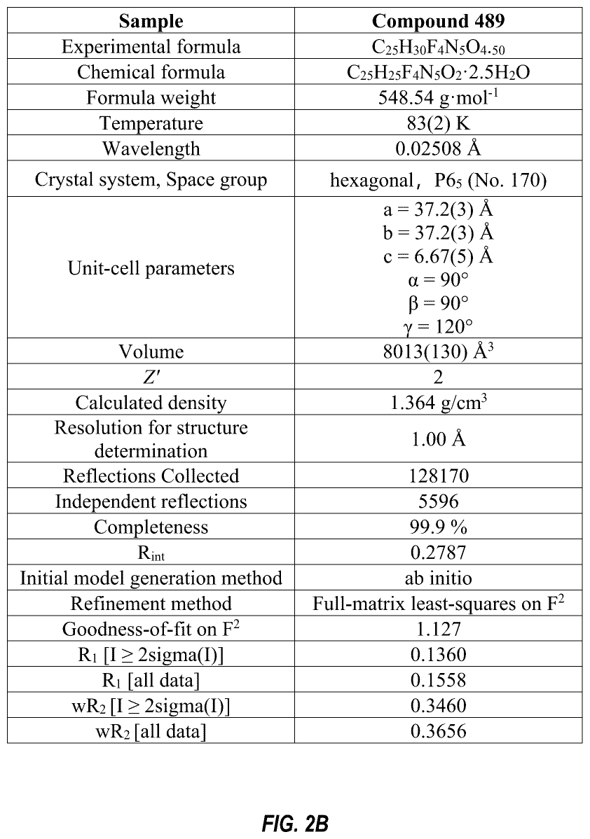

But, in some instances, the absolute stereo configurations of one or more chiral centers are arbitrarily assigned (e.g., stereochemistry of one chiral center is known and remaining chiral centers arbitrarily assigned). Accordingly, the enantiomers or diastereomers are identified by their respective properties, for example, retention times on a prep chiral HPLC, chiral SFC, NMR shift or optical rotation or its biological activities (e.g., as described further in the Examples). Thus, should the stereochemistry assigned to any compound or compounds ultimately be proven incorrect, then the analytical data (e.g., prep chiral HPLC, chiral SFC, NMR shift or optical rotation or biological activity) associated with each compound is determinative of the actual identity of the compound. In addition, in light of such corrected stereochemical designation appropriate adjustments to the stereochemistry identification contained in the description, examples, tables and claims should be adjusted as needed by one skilled in the art. For example, the stereochemistry of Example 53 compound 489 (see FIGS. 1A, 1B, 2A) has been determined by MicroED utilizing vapor diffusion in ACN/DIPE (as described in Example 53 Step 8) to be 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (Compound 489). The absolute stereochemistry of the other compounds has not been determined by MicroED.

Chiral analytical separation methods (e.g., supercritical fluid chromatography (SFC), and high performance liquid chromatography (HPLC)) used in the following synthetic examples are summarized in Table A below. These methods were used to identify a single compound/stereoisomer from a mixture of chiral compounds based on a peak retention time.

TABLE A

| | | | | | | Column | Back | |
|---|---|---|---|---|---|---|---|---|
| Method | Column | Mobile Phase | Flow Type | Flow Rate | | Temp. | Pressure | |
| A | Cellulose SZ, | A: Hex (0.1% | Isocratic: | 1.67 | mL/min | Ambient | | |
| | 0.46 × 5 cm, 3 μM | DEA) B: EtOH | 50% B | | | | | |
| B | CHIRALPAK IC-3, | A: MtBE (0.1% | Isocratic: | 1.67 | mL/min | Ambient | | |
| | 0.46 × 5 cm, 3 μM | DEA) B: MeOH | 50% B | | | | | |
| C | CHIRALPAK IA-3, | A: MtBE (0.1% | Isocratic: | 1.67 | mL/min | Ambient | | |
| | 0.46 × 5 cm, 3 μM | DEA) B: MeOH | 50% B | | | | | |
| D | CHIRALPAK IE-3, | A: (MtBE:Hex = | Isocratic: | 1.67 | mL/min | Ambient | | |
| | 0.46 × 5 cm, 3 μM | 1:1) (0.1% DEA) | 15% B | | | | | |
| | | B: MeOH | | | | | | |
| E | CHIRALPAK IE-3, | A: MtBE (0.1% | Isocratic: | 1.67 | mL/min | Ambient | | |
| | 0.46 × 5 cm, 3 μM | DEA) B: MeOH | 50% B | | | | | |
| F | CHIRAL ART | A: CO2 B: MeOH | Gradient: | 2.0 | mL/min | 35° C. | 110 | bar |
| | Cellulose-SB, | (20 mM NH3) | 10% to 50% | | | | | |
| | 0.46 × 5 cm, 3 μM | | B in 4 min; | | | | | |
| | | | hold at 50% | | | | | |
| | | | B for 2 min | | | | | |
| G | IDA-3, | A: MtBE (0.1% | Isocratic: | 1.67 | mL/min | Ambient | | |
| | 0.46 × 5 cm, 3 μM | DEA) B: MeOH | 50% B | | | | | |
| H | CHIRALPAK IE-3, | A: MtBE (0.1% | Isocratic: | 1.67 | mL/min | Ambient | | |
| | 0.46 × 5 cm, 3 μM | DEA) B: MeOH | 20% B | | | | | |
| I | Cellulose SZ, | A: Hex (0.1% | Isocratic: | 1.67 | mL/min | Ambient | | |
| | 0.46 × 5 cm, 3 μM | DEA) B: EtOH | 30% B | | | | | |
| J | Cellulose SB, | A: MtBE (0.1% | Isocratic: | 1.67 | mL/min | Ambient | | |
| | 0.46 × 5 cm, 3 μM | DEA) B: MeOH | 10% B | | | | | |
| K | Cellulose SZ, | A: Hex (0.1% | Isocratic: | 1.67 | mL/min | Ambient | | |
| | 0.46 × 5 cm, 3 μM | DEA) B: EtOH | 20% B | | | | | |
| L | IDA-3, | A: Hex (0.1% | Isocratic: | 1.67 | mL/min | Ambient | | |
| | 0.46 × 5 cm, 3 μM | DEA) B: EtOH | 50% B | | | | | |
| M | CHIRALPAK ID-3, | A: MtBE (0.1% | Isocratic: | 1.67 | mL/min | Ambient | | |
| | 0.46 × 5 cm, 3 μM | TFA) B: MeOH | 15% B | | | | | |
| N | CHIRAL Cellulose SB, | A: MtBE (0.1% | Isocratic: | 1.67 | mL/min | Ambient | | |
| | 0.46 × 5 cm, 3 μM | TFA) B: MeOH | 15% B | | | | | |
| O | CHIRALPAK IF-3, | A: Hex (10 mM | Isocratic: | 1.0 | mL/min | Ambient | | |
| | 0.46 × 5 cm, 3 μM | NH3) B: EtOH | 50% B | | | | | |
| P | CHIRALPAK ID-3, | A: MtBE (0.1% | Isocratic: | 1.67 | mL/min | Ambient | | |
| | 0.46 × 5 cm, 3 μM | DEA) B: MeOH | 30% B | | | | | |
| Q | CHIRALPAK IC-3, | A: MtBE (0.1% | Isocratic: | 1.67 | mL/min | Ambient | | |
| | 0.46 × 5 cm, 3 μM | DEA) B: MeOH | 30% B | | | | | |
| R | CHIRAL Cellulose SB, | A: MtBE (0.1% | Isocratic: | 1.67 | mL/min | Ambient | | |
| | 0.46 × 5 cm, 3 μM | DEA) B: MeOH | 20% B | | | | | |
| S | Cellulose SZ, | A: Hex (0.1% | Isocratic: | 1.67 | mL/min | Ambient | | |
| | 0.46 × 5 cm, 3 μM | DEA) B: EtOH | 30% B | | | | | |
| T | CHIRAL Cellulose SB, | A: MtBE (0.1% | Isocratic: | 1.67 | mL/min | Ambient | | |
| | 0.46 × 5 cm, 3 μM | DEA) B: MeOH | 15% B | | | | | |
| U | CHIRALPAK IH-3, | A: CO2 B: | Isocratic: | 2.0 | mL/min | 35° C. | 110 | bar |
| | 0.3 x 10 cm, 3 μM | MeOH (0.1% | 50% B | | | | | |
| | | DEA) | | | | | | |
| V | Cellulose SB, | A: Hex (0.1% | Isocratic: | 1.67 | mL/min | Ambient | | |
| | 0.46 × 5 cm, 3 μM | DEA) B: EtOH | 50% B | | | | | |
| W | Cellulose SZ, | A: MtBE (0.1% | Isocratic: | 1.0 | mL/min | Ambient | | |
| | 0.46 × 5 cm, 3 μM | DEA) B: MeOH | 25% B | | | | | |
| X | Lux 3μ Cellulose-2, | A: Hex (0.1% | Isocratic: | 1.0 | mL/min | Ambient | | |
| | 0.46 × 5 cm, 3 μM | DEA) B: EtOH | 50% B | | | | | |
| Y | CHIRALPAK IK-3, | A: Hex (0.1% | Isocratic: | 1.67 | mL/min | Ambient | | |
| | 0.46 × 5 cm, 3 μM | DEA) B: EtOH | 50% B | | | | | |
| Z | Enantiocel C9-3, | A: CO2 B: MeOH | Gradient: | 2.0 | mL/min | 35° C. | 110 | bar |
| | 0.46 × 5 cm, 3 μM | (0.1% DEA) | 10% to 50% | | | | | |
| | | | B in 4 min; | | | | | |
| | | | hold at 50% | | | | | |
| | | | B for 2 min | | | | | |
| AA | CHIRALPAK IC-3, | A: Hex (0.1% | Isocratic: | 1.0 | mL/min | Ambient | | |
| | 0.46 × 5 cm, 3 μM | DEA) B: EtOH | 20% B | | | | | |
| AB | CHIRALPAK IK-3, | A: Hex (0.1% | Isocratic: | 1.67 | mL/min | Ambient | | |
| | 0.46 × 5 cm, 3 μM | DEA) B: EtOH | 10% B | | | | | |
| AC | Cellulose SZ, | A: Hex (0.1% | Isocratic: | 1.67 | mL/min | Ambient | | |
| | 0.46 × 5 cm, 3 μM | DEA) B: EtOH | 10% B | | | | | |
| AD | Lux 3μ Cellulose-2, | A: Hex (0.1% | Isocratic: | 1.0 | mL/min | Ambient | | |
| | 0.46 × 5 cm, 3 μM | DEA) B: | 50% B | | | | | |
| | | (EtOH:ACN = 5:1) | | | | | | |
| AE | CHIRALPAK IC-3, | A: MtBE (0.1% | Isocratic: | 1.67 | mL/min | Ambient | | |
| | 0.46 × 5 cm, 3 μM | DEA) B: EtOH | 30% B | | | | | |
| AF | Lux 3μ Cellulose-2, | A: Hex (0.1% | Isocratic: | 1.67 | mL/min | Ambient | | |
| | 0.46 × 5 cm, 3 μM | DEA) B: EtOH | 50% B | | | | | |
| AG | CHIRALCEL OD-3, | A: Hex (0.1% | Isocratic: | 1.00 | mL/min | Ambient | | |
| | 0.46 × 5 cm, 3 μM | DEA) B: EtOH | 30% B | | | | | |
| AH | Cellulose SZ, | A: MtBE (0.1% | Isocratic: | 1.67 | mL/min | Ambient | | |
| | 0.46 × 5 cm, 3 μM | DEA) B: MeOH | 50% B | | | | | |
| AI | CHIRALPAK IK-3, | A: CO2 B: MeOH | Gradient:10% | 2.0 | mL/min | 35° C. | 1500 | psi |
| | 0.46 × 10 cm, 3 μM | (0.1% DEA) | to 50% B in 2 | | | | | |
| | | | min, hold 1.0 | | | | | |
| | | | min at 50% B | | | | | |
| AJ | CHIRALPAK IK-3, | A: MtBE (0.1% | Isocratic: | 1.67 | mL/min | Ambient | | |
| | 0.46 × 5 cm, 3 μM | DEA) B: EtOH | 50% B | | | | | |
| AK | Lux Cellulose-4, | A: CO2 B: MeOH | Gradient: | 4.0 | mL/min | 35° C. | 1500 | psi |
| | 0.46 × 5 cm, 3 μM | (0.1% DEA) | 10% to 50% | | | | | |
| | | | B in 2 min, | | | | | |
| | | | hold 1.0 min | | | | | |
| | | | at 50% B | | | | | |
| AL | CHIRALPAK IM-3, | A: CO2 B: MeOH | Gradient: | 4.0 | mL/min | 35° C. | 1500 | psi |
| | 0.46 × 10 cm, 3 μM | (0.1% DEA) | 10% to 50% | | | | | |
| | | | B in 2 min, | | | | | |
| | | | hold 1.0 min | | | | | |
| | | | at 50% B | | | | | |
| AM | CHIRALPAK IK-3, | A: CO2 B: MeOH | Isocratic: | 4.0 | mL/min | 35° C. | 1500 | psi |
| | 0.46 × 10 cm, 3 μM | (0.1% DEA) | 50% B | | | | | |
| AN | Enantiocel C4-3, | A: Hex (0.1% | Isocratic: | 1.67 | mL/min | Ambient | | |
| | 0.46 × 5 cm, 3 μM | DEA) B: EtOH | 30% B | | | | | |
| AO | Lux 3μ Cellulose-2, | A: Hex (0.1% | Isocratic: | 1.67 | mL/min | Ambient | | |
| | 0.46 × 5 cm, 3 μM | DEA) B: EtOH | 30% B | | | | | |
| AP | CHIRAL Cellulose-SB, | A: MtBE (0.1% | Isocratic: | 1.67 | mL/min | Ambient | | |
| | 0.46 × 5 cm, 3 μM | DEA) B: MeOH | 10% B | | | | | |
| AQ | Lux 3μ Cellulose-2, | A: Hex (0.1% | Isocratic: | 1.67 | mL/min | Ambient | | |
| | 0.46 × 5 cm, 3 μM | DEA) B: | 50% B | | | | | |
| | | (EtOH:ACN = 5:1) | | | | | | |

### Synthesis of Intermediate B & C: (S)-4-(4,4-difluoropiperidin-3-yl)pyridine 1-oxide and (R)-4-(4,4-difluoropiperidin-3-yl)pyridine 1-oxide

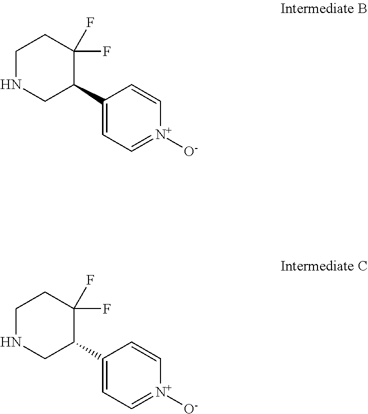 [SMILES: [O-][n+]1ccc([C@H]2CNCCC2(F)F)cc1 ; [O-][n+]1ccc([C@@H]2CNCCC2(F)F)cc1]

### Step 1: tert-butyl 4-oxo-3-(pyridin-4-yl)piperidine-1-carboxylate

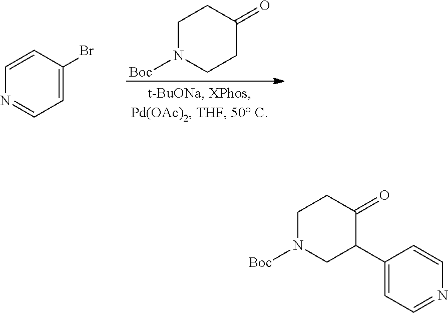 [SMILES: Brc1ccncc1 ; CC(C)(C)OC(=O)N1CCC(=O)CC1 ; CC(C)(C)OC(=O)N1CCC(=O)C(c2ccncc2)C1]

A mixture of t-BuONa (19.8 g, 205.69 mmol), XPhos (4.9 g, 10.28 mmol), Pd(OAc)₂ (1.2 g, 5.14 mmol), 4-bromopyridine (10.0 g, 63.29 mmol) and tert-butyl 4-oxopiperidine-1-carboxylate (14.3 g, 71.99 mmol) in THF (200 mL) was stirred at 50° C. overnight under a nitrogen atmosphere. The mixture was allowed to cool down to room temperature. The reaction was diluted with water at 0° C. The aqueous layer was extracted with EtOAc (3×300 mL). The resulting mixture was concentrated under reduced pressure. The residue was purified by silica gel column chromatography, eluted with CH₂Cl₂/EA (1:1) to afford tert-butyl 4-oxo-3-(pyridin-4-yl)piperidine-1-carboxylate (9.0 g, 63% yield) as an off-white solid. LCMS (ESI) [M+H]⁺: 277.

### Step 2: tert-butyl 4,4-difluoro-3-(pyridin-4-yl)piperidine-1-carboxylate

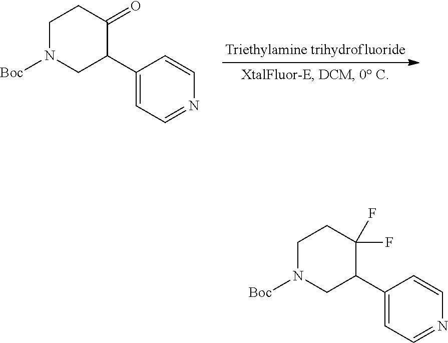 [SMILES: CC(C)(C)OC(=O)N1CCC(=O)C(c2ccncc2)C1 ; CC(C)(C)OC(=O)N1CCC(F)(F)C(c2ccncc2)C1]

To a mixture of triethylamine trihydrofluoride (11.0 g, 68.03 mmol) and XtalFluor-E (23.4 g, 102.05 mmol) in DCM (100 ml) was added tert-butyl 4-oxo-3-(pyridin-4-yl)piperidine-1-carboxylate (9.4 g, 34.01 mmol) in portions at 0° C. under a nitrogen atmosphere. The resulting mixture was stirred at 0° C. overnight under a nitrogen atmosphere. The reaction was quenched with sat. aq. NaHCO₃ (50 mL) at 0° C. The resulting mixture was extracted with CH₂Cl₂ (3×150 mL), dried over anhydrous Na₂SO₄ and filtered. The filtrate was concentrated under reduced pressure. The residue was purified by silica gel column chromatography, eluted with PE/EA (1:1) to afford tert-butyl 4,4-difluoro-3-(pyridin-4-yl)piperidine-1-carboxylate (3.0 g, 29% yield) as a yellow oil. LCMS (ESI) [M+H]⁺: 299.

### Step 3: 4-(1-(tert-butoxycarbonyl)-4,4-difluoropiperidin-3-yl)pyridine 1-oxide

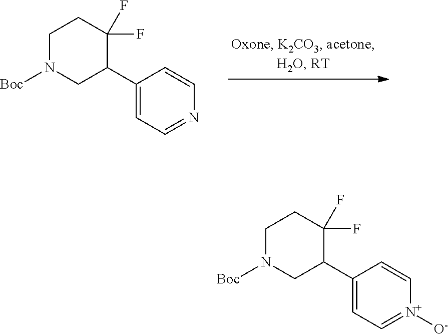 [SMILES: CC(C)(C)OC(=O)N1CCC(F)(F)C(c2ccncc2)C1 ; CC(C)(C)OC(=O)N1CCC(F)(F)C(c2cc[n+]([O-])cc2)C1]

To a mixture of tert-butyl 4,4-difluoro-3-(pyridin-4-yl)piperidine-1-carboxylate (3.0 g, 10.05 mmol) and K₂CO₃ (4.2 g, 30.16 mmol) in 1:1 acetone/water (70 mL) was added Oxone (3.4 g, 20.11 mmol) in portions at 0° C. The resulting mixture was stirred at room temperature overnight. The reaction was quenched with sat. aq. sodium hyposulfite (50 mL) at 0° C. The resulting mixture was extracted with CH₂Cl₂ (3×100 mL). Dried over anhydrous Na₂SO₄ and filtered. The filtrate was concentrated under reduced pressure. The residue was purified by silica gel column chromatography, eluted with CH₂Cl₂/MeOH (7:1) to afford 4-(1-(tert-butoxycarbonyl)-4,4-difluoropiperidin-3-yl)pyridine 1-oxide (2.6 g, 82% yield) as a yellow oil.

LCMS (ESI) [M+H]⁺: 315

### Step 4: 4-(4,4-difluoropiperidin-3-yl)pyridine 1-oxide

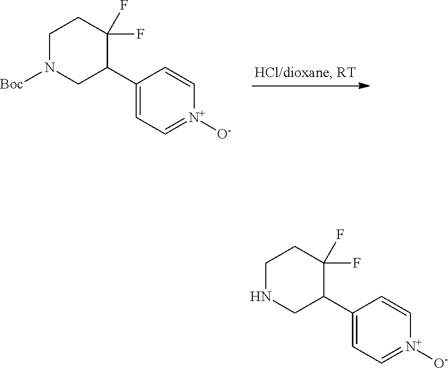 [SMILES: CC(C)(C)OC(=O)N1CCC(F)(F)C(c2cc[n+]([O-])cc2)C1 ; [O-][n+]1ccc(C2CNCCC2(F)F)cc1]

A mixture of 4-(1-(tert-butoxycarbonyl)-4,4-difluoropiperidin-3-yl)pyridine 1-oxide (2.6 g, 8.27 mmol) in 4M HCl in 1,4-dioxane (24 mL) was stirred at room temperature overnight. The resulting mixture was concentrated under reduced pressure. The residue was purified by reversed-phase flash chromatography with the following conditions: column, C18; mobile phase, MeCN in water (10 mmol/L NH₄HCO₃), 5% to 100% gradient in 20 min; detector, UV 254 nm and UV 220 nm. This resulted in 4-(4,4-difluoropiperidin-3-yl)pyridine 1-oxide (1.7 g, 95% yield) as a yellow solid. LCMS (ESI) [M+H]⁺: 215.

### Step 5: (S)-4-(4,4-difluoropiperidin-3-yl)pyridine 1-oxide and (R)-4-(4,4-difluoropiperidin-3-yl)pyridine-1-oxide

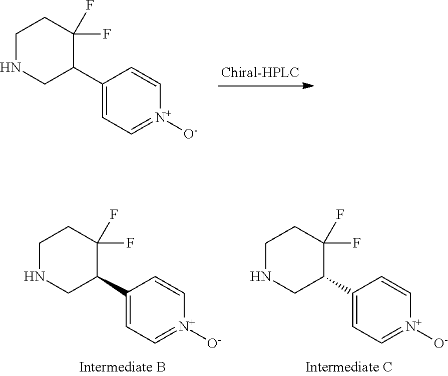 [SMILES: [O-][n+]1ccc(C2CNCCC2(F)F)cc1 ; [O-][n+]1ccc([C@H]2CNCCC2(F)F)cc1 ; [O-][n+]1ccc([C@@H]2CNCCC2(F)F)cc1]

4-(4,4-difluoropiperidin-3-yl)pyridine 1-oxide (500 mg) was separated by prep-Chiral-HPLC (Column: CHIRALPAK IG, 3×25 cm, 5 μm; Mobile Phase A: CO₂, Mobile Phase B: MeOH (0.1% 2M NH₃-MeOH); Flow rate: 90 mL/min; Gradient: isocratic 50% B; Column Temperature (° C.): 35; Back Pressure (bar): 120; Wave Length: 270/250 nm; RT1 (min): 4; RT2 (min): 7.28) to afford (S)-4-(4,4-difluoropiperidin-3-yl)pyridine 1-oxide (180 mg, 100% e.e.) (Intermediate B) and (R)-4-(4,4-difluoropiperidin-3-yl)pyridine 1-oxide (310 mg, 100% e.e.) (Intermediate C). The isomers are tentatively assigned by analogy to the known stereochemistry in example 132 in WO2202/073904. LCMS (ESI) [M+H]⁺: 215.

### Synthesis of Intermediate D & E: 4-((S)-1-((S)-1-((1H-imidazol-4-yl)amino)-1-oxopropan-2-yl)-4,4-difluoropiperidin-3-yl)pyridine 1-oxide and 4-((S)-1-((R)-1-((1H-imidazol-4-yl)amino)-1-oxopropan-2-yl)-4,4-difluoropiperidin-3-yl)pyridine 1-oxide

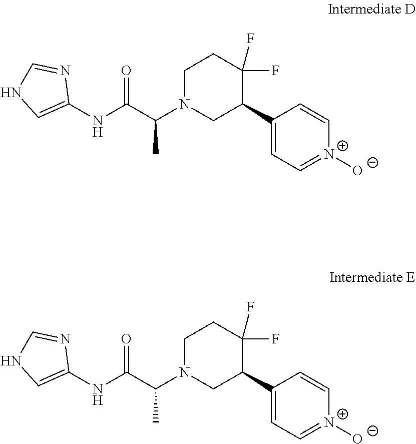 [SMILES: C[C@@H](C(=O)Nc1c[nH]cn1)N1CCC(F)(F)[C@@H](c2cc[n+]([O-])cc2)C1 ; C[C@H](C(=O)Nc1c[nH]cn1)N1CCC(F)(F)[C@@H](c2cc[n+]([O-])cc2)C1]

### Step 1: 4-nitro-1-trityl-1H-imidazole

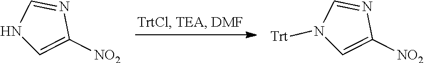

To a mixture of 4-nitro-1H-imidazole (10 g, 88.44 mmol) and triethylamine (16 g, 154 mmol) in DMF (20 mL) was added triphenylmethyl chloride (24.5 g, 88.44 mmol) in portions at 0° C. under a nitrogen atmosphere. The resulting mixture was stirred at 25° C. for 2 hours. The mixture was poured into ice water and filtered. The filter cake was collected and air dried to afford 4-nitro-1-trityl-1H-imidazole (28 g, 89% yield) as a white solid. LCMS (ESI) [M+H]⁺ 356.13

### Step 2: 1-trityl-1H-imidazol-4-amine

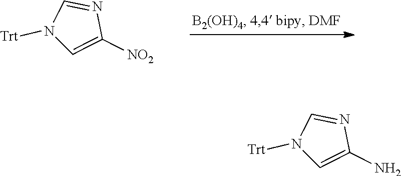

A solution of tetrahydroxydiboron (20 g, 224 mmol) in DMF (20 mL) was slowly added into a solution of 4-nitro-1-trityl-1H-imidazole (20 g, 56.28 mmol) and 4, 4′ bipyridine (1 g, 5.6 mmol) in DMF (40 mL) at 0° C. Upon completion of the addition, the solution turned purple and was allowed to warm back to room temperature. The solution was stirred for another 30 minutes and became colorless again. TLC suggested complete consumption of the starting material. The mixture was poured into water (200 mL) and extracted with EA (3×200 mL). The organic layers were separated, combined, dried over MgSO₄, filtered and concentrated under vacuum to give crude 1-trityl-1H-imidazol-4-amine (18 g, 98% quant), which was used directly in the next step directly without further purification.

### Step 3: 2-bromo-N-(1-trityl-1H-imidazol-4-yl)propanamide

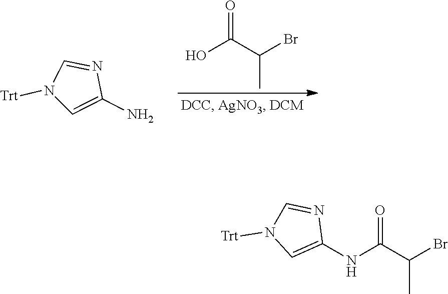

A mixture of 1-trityl-1H-imidazol-4-amine (12 g, 30.73 mmol), 2-bromopropanoic acid (18.8 g, 122.92 mmol), DCC (18.54 g, 90 mmol) and AgNO₃ (1 g, 3.3 mmol) in DCM (50 mL) was stirred overnight at room temperature. The resulting mixture was filtered and the precipitate was washed with DCM (3×10 mL). The resulting mixture was concentrated under vacuum. The residue was purified by silica gel column chromatography, eluted with PE/EA (1:1) to afford 2-bromo-N-(1-trityl-1H-imidazol-4-yl)propanamide (15.3 g, 87% yield) as a white solid. LCMS (ESI) [M+H]⁺: 460.09.

### Step 4: 4-((3S)-4,4-difluoro-1-(1-oxo-1-((1-trityl-1H-imidazol-4-yl)amino)propan-2-yl)piperidin-3-yl)pyridine 1-oxide

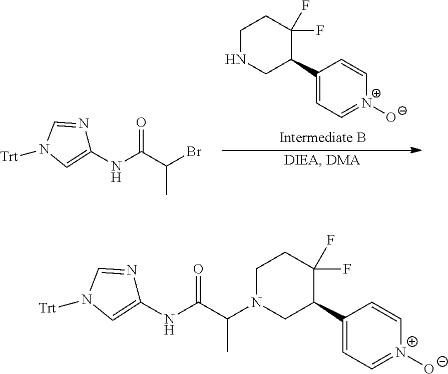

Into a 8-mL sample vial was placed 2-bromo-N-(1-trityl-1H-imidazol-4-yl)propanamide (14 g, 30.41 mmol), Intermediate B (6.51 g, 30.41 mmol), N, N-diisopropylethylamine (16 mL) and DMA (20 mL). The mixture was stirred at room temperature for 24 h until LCMS suggested the completion of the reaction. The resulting solution was allowed to cool down to room temperature, poured into water (10 mL) and extracted with EA (3×10 mL). The organic layers were combined, dried over MgSO₄, filtered and concentrated. The crude residue was purified by silica gel chromatography (methanol:dichloromethane=1:15) to give 4-((3S)-4,4-difluoro-1-(1-oxo-1-((1-trityl-1H-imidazol-4-yl)amino)propan-2-yl)piperidin-3-yl)pyridine 1-oxide (14.5 g, 77%) as a white solid. LCMS (ESI) [M+H]⁺: 594.

### Step 5: 4-((3S)-1-(1-((1H-imidazol-4-yl)amino)-1-oxopropan-2-yl)-4,4-difluoropiperidin-3-yl)pyridine 1-oxide

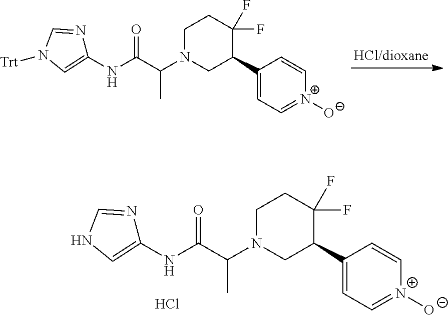

HCl (10 mL, 20 mmol, 2M in dioxane) was added into a 50 mL round bottom flask containing 4-((3S)-4,4-difluoro-1-(1-oxo-1-((1-trityl-1H-imidazol-4-yl)amino)propan-2-yl)piperidin-3-yl)pyridine 1-oxide (14.5 g, 24.4 mmol). The mixture was stirred at 25° C. for 3 hours until LCMS suggested the completion of the reaction. The mixture was concentrated and triturated with dioxane (3×10 mL) to give 4-((3S)-1-(1-((1H-imidazol-4-yl)amino)-1-oxopropan-2-yl)-4,4-difluoropiperidin-3-yl)pyridine 1-oxide (9.1 g, 95%) as a white solid.

LCMS (ESI) [M+H]⁺: 352.

### Step 6: 4-((S)-1-((S)-1-((1H-imidazol-4-yl)amino)-1-oxopropan-2-yl)-4,4-difluoropiperidin-3-yl)pyridine 1-oxide and 4-((S)-1-((R)-1-((1H-imidazol-4-yl)amino)-1-oxopropan-2-yl)-4,4-difluoropiperidin-3-yl)pyridine 1-oxide

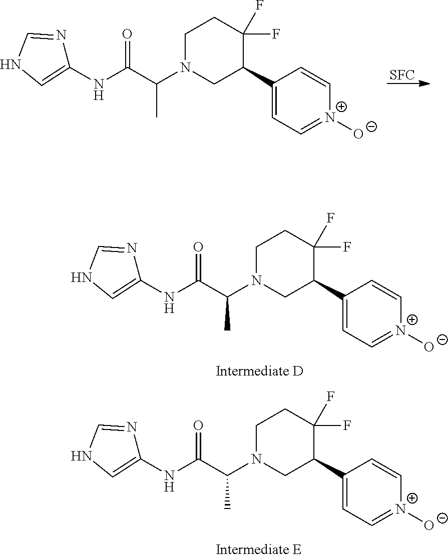

4-((3S)-1-(1-((1H-imidazol-4-yl)amino)-1-oxopropan-2-yl)-4,4-difluoropiperidin-3-yl)pyridine 1-oxide (12 g) was purified by Prep-Chiral SFC with the following conditions Column: CHIRALPAK IG, 3×25 cm, 5 μm; Mobile Phase A: CO₂, Mobile Phase B: MeOH:DCM=1:1 (20 mM NH₃); Flow rate: 40 mL/min; Gradient: isocratic 40% B; Column Temperature (° C.): 35; Back Pressure (bar): 90; Wave Length: 210/238 nm; RT1 (min): 4.58; RT2 (min): 6.37; Sample Solvent: MeOH:DCM=1:1; Injection Volume: 0.6 mL to afford 4-((S)-1-((S)-1-((1H-imidazol-4-yl)amino)-1-oxopropan-2-yl)-4,4-difluoropiperidin-3-yl)pyridine 1-oxide (Intermediate D) (5.3 g, 100% e.e.) as a white solid and 4-((S)-1-((R)-1-((1H-imidazol-4-yl)amino)-1-oxopropan-2-yl)-4,4-difluoropiperidin-3-yl)pyridine 1-oxide (Intermediate E) (3.6 g, 100% e.e.) as a white solid.

### Example 1: 4-(1-(1-((1-(2,4-difluorobenzyl)-1H-imidazol-4-yl)amino)-1-oxopropan-2-yl)-4,4-difluoropiperidin-3-yl)pyridine 1-oxide

### Step 1: 1-(2,4-difluorobenzyl)-4-nitro-1H-imidazole

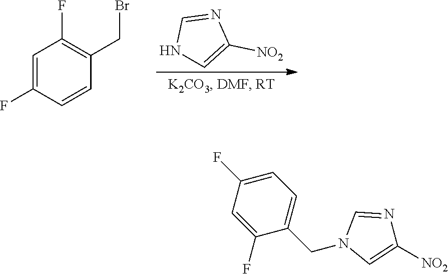 [SMILES: Fc1ccc(CBr)c(F)c1 ; O=[N+]([O-])c1c[nH]cn1 ; O=[N+]([O-])c1cn(Cc2ccc(F)cc2F)cn1]

A mixture of 1-(bromomethyl)-2,4-difluorobenzene (500 mg, 2.42 mmol) and 4-nitroimidazole (350 mg, 3.10 mmol), K₂CO₃ (1.0 g, 7.24 mmol) in DMF (10 mL) was stirred for 1 hour at room temperature. The reaction was quenched with water. The resulting mixture was extracted with EtOAc (3×15 mL). The combined organic layers were washed with brine (15 mL), dried over anhydrous Na₂SO₄ and filtered. The resulting mixture was concentrated under vacuum. The residue was purified by silica gel column chromatography, eluted with PE/EA (1:1) to afford 1-(2,4-difluorobenzyl)-4-nitro-1H-imidazole (500 mg, 86% yield) as a white solid. LCMS (ESI) [M+H]⁺: 240.

### Step 2: 1-(2,4-difluorobenzyl)-1H-imidazol-4-amine

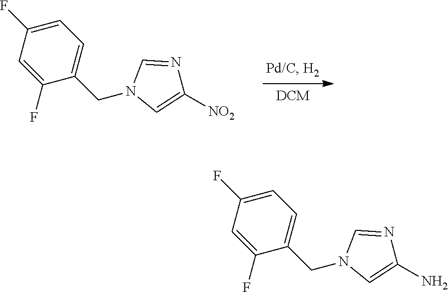 [SMILES: O=[N+]([O-])c1cn(Cc2ccc(F)cc2F)cn1 ; Nc1cn(Cc2ccc(F)cc2F)cn1]

A mixture of 1-(2,4-difluorobenzyl)-4-nitro-1H-imidazole (500 mg, 2.09 mmol) and 10% Pd/C (50 mg, anhydrous) in DCM (10 mL) was stirred for 1 hour at room temperature under a hydrogen atmosphere (2 ATM). The resulting mixture was filtered and the filter cake was washed with DCM (3×15 mL). The filtrate was concentrated under reduced pressure to afford 1-(2,4-difluorobenzyl)-1H-imidazol-4-amine (400 mg, crude) as a yellow oil which was used in the next step without further purification. LCMS (ESI) [M+H]⁺: 210.

### Step 3: (2R)-2-Bromo-N-(1-(2,4-difluorobenzyl)-1H-imidazol-4-yl)propanamide

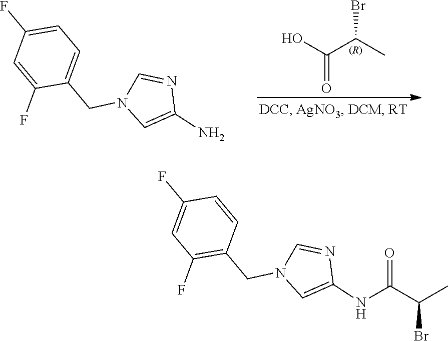

A mixture of 1-(2,4-difluorobenzyl)-1H-imidazol-4-amine (400 mg, 1.91 mmol), (2R)-2-bromopropanoic acid (351 mg, 2.29 mmol), DCC (473 mg, 2.29 mmol) and AgNO₃ (65 mg, 0.38 mmol) in DCM (8 mL) was stirred overnight at room temperature. The resulting mixture was filtered and the filter cake was washed with DCM (3×10 mL). The resulting mixture was concentrated under vacuum. The residue was purified by silica gel column chromatography, eluted with PE/EA (1:1) to afford (2R)-2-Bromo-N-(1-(2,4-difluorobenzyl)-1H-imidazol-4-yl)propanamide (270 mg, 41% yield) as a white solid. LCMS (ESI) [M+H]⁺: 344.

### Step 4: 4-((3S)-1-(1-((1-(2,4-difluorobenzyl)-1H-imidazol-4-yl)amino)-1-oxopropan-2-yl)-4,4-difluoropiperidin-3-yl)pyridine 1-oxide

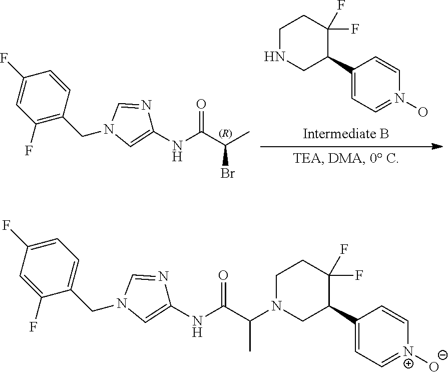

A mixture of (2R)-2-bromo-N-{1-[(2,4-difluorophenyl)methyl]imidazol-4-yl}propanamide (100 mg, 0.29 mmol), (S)-4-(4,4-difluoropiperidin-3-yl)pyridine 1-oxide (Intermediate B) (52 mg, 0.24 mmol) and TEA (74 mg, 0.73 mmol) in DMA (3 mL) was stirred for 2 days at room temperature. The resulting mixture was purified by Prep-HPLC with the following conditions (Column: XSelect CSH Prep C18 OBD Column, 30×150 mm, 5 μm; Mobile Phase A: Water (0.1% FA), Mobile Phase B: ACN; Flow rate: 60 mL/min; Gradient: 10% B to 30% B in 10 min; Wave Length: 254 nm/220 nm; RT1 (min): 12.28) to afford 4-((3S)-1-(1-((1-(2,4-difluorobenzyl)-1H-imidazol-4-yl)amino)-1-oxopropan-2-yl)-4,4-difluoropiperidin-3-yl)pyridine 1-oxide (50 mg, 36% yield) as a white solid. LCMS (ESI) [M+H]⁺: 478.

### Step 5: 4-((S)-1-((S)-1-((1-(2,4-Difluorobenzyl)-1H-imidazol-4-yl)amino)-1-oxopropan-2-yl)-4,4-difluoropiperidin-3-yl)pyridine 1-oxide and 4-((S)-1-((R)-1-((1-(2,4-difluorobenzyl)-1H-imidazol-4-yl)amino)-1-oxopropan-2-yl)-4,4-difluoropiperidin-3-yl)pyridine 1-oxide

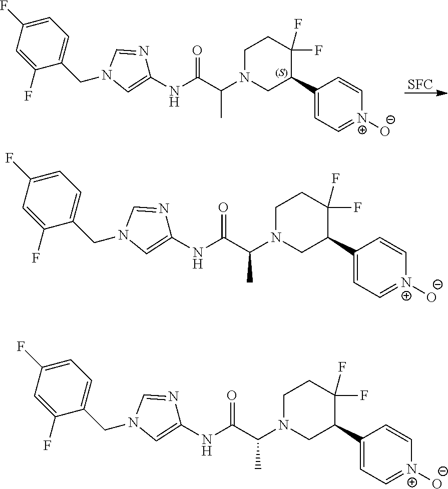

4-((3S)-1-(1-((1-(2,4-difluorobenzyl)-1H-imidazol-4-yl)amino)-1-oxopropan-2-yl)-4,4-difluoropiperidin-3-yl)pyridine 1-oxide (50 mg) was purified by Prep-Chiral HPLC with the following conditions (Column: CHIRAL ART Cellulose-SZ, 3×25 cm, 5 μm; Mobile Phase A: Hex (0.1% DEA), Mobile Phase B: EtOH; Flow rate: 40 mL/min; Gradient: isocratic 50% B; Wave Length: 212/268 nm; RT1 (min): 27.06; RT2 (min): 30.17; Sample Solvent: EtOH; Injection Volume: 0.5 mL; Number Of Runs: 3) to afford 4-((S)-1-((S)-1-((1-(2,4-Difluorobenzyl)-1H-imidazol-4-yl)amino)-1-oxopropan-2-yl)-4,4-difluoropiperidin-3-yl)pyridine 1-oxide (Compound 1) (Method A, 2.07 min, peak 1, 17.7 mg, 100% e.e.) as a white solid and 4-((S)-1-((R)-1-((1-(2,4-difluorobenzyl)-1H-imidazol-4-yl)amino)-1-oxopropan-2-yl)-4,4-difluoropiperidin-3-yl)pyridine 1-oxide (Compound 2) (Method A, 2.65 min, peak 2, 3 mg, 100% e.e.) as a white solid.

TABLE 1

| Compound# | Characterization Data |
|---|---|
| 1 | LCMS (ESI) [M + H]⁺: 478.2. |
| | ¹H NMR (400 MHz, Methanol-d₄) δ 8.28 (d, J = 6.8 Hz, 2H), 7.56 (d, J = 7.2 Hz, 3H), |
| | 7.37 (td, J = 8.5, 6.3 Hz, 1H), 7.27 (s, 1H), 7.04-6.96 (m, 2H), 5.21 (s, 2H), 3.64-3.52 |
| | (m, 1H), 3.50 (q, J = 7.0 Hz, 1H), 3.00 (d, J = 9.2 Hz, 2H), 2.91 (d, J = 12.1 Hz, 1H), |
| | 2.64-2.59 (m, 1H), 2.37-2.16 (m, 2H), 1.31 (d, J = 7.0 Hz, 3H). |
| 2 | LCMS (ESI) [M + H]⁺: 478.2. |
| | ¹H NMR (400 MHz, Methanol-d₄) δ 8.27 (d, J = 6.8 Hz, 2H), 7.65-7.52 (m, 3H), 7.38 |
| | (td, J = 8.5, 6.3 Hz, 1H), 7.27 (d, J = 1.6 Hz, 1H), 7.08-6.93 (m, 2H), 5.21 (s, 2H), |
| | 3.73-3.54 (m, 1H), 3.49 (q, J = 7.0 Hz, 1H), 3.02-2.92 (m, 2H), 2.93 (d, J = 11.8 Hz, 1H), |
| | 2.87-2.76 (m, 2H), 2.39-2.12 (m, 2H), 1.31 (d, J = 7.0 Hz, 3H). |

### Step 6: 4-((3R)-1-(1-((1-(2,4-difluorobenzyl)-1H-imidazol-4-yl)amino)-1-oxopropan-2-yl)-4,4-difluoropiperidin-3-yl)pyridine 1-oxide

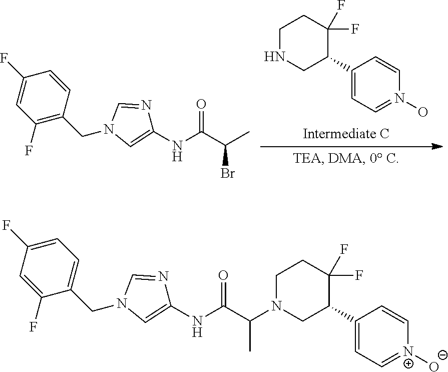

A mixture of (2R)-2-bromo-N-{1-[(2,4-difluorophenyl)methyl]imidazol-4-yl}propanamide (100 mg, 0.29 mmol), (R)-4-(4,4-difluoropiperidin-3-yl)pyridine 1-oxide (52 mg, 0.24 mmol) (Intermediate C) and TEA (74 mg, 0.73 mmol) in DMA (3 mL) was stirred for 2 days at room temperature. The crude product was purified by Prep-HPLC with the following conditions (Column: XSelect CSH Prep C18 OBD Column, 30×150 mm, 5 μm; Mobile Phase A: Water (0.1% FA), Mobile Phase B: ACN; Flow rate: 60 mL/min; Gradient: 10% B to 30% B in 10 min; Wave Length: 254 nm/220 nm; RT1 (min): 12.28) to afford 4-((3R)-1-(1-((1-(2,4-difluorobenzyl)-1H-imidazol-4-yl)amino)-1-oxopropan-2-yl)-4,4-difluoropiperidin-3-yl)pyridine 1-oxide (50 mg, 36% yield) as a white solid. LCMS (ESI) [M+H]⁺: 478.

### Step 7: 4-((R)-1-((S)-1-((1-(2,4-difluorobenzyl)-1H-imidazol-4-yl)amino)-1-oxopropan-2-yl)-4,4-difluoropiperidin-3-yl)pyridine 1-oxide and 4-((R)-1-((R)-1-((1-(2,4-difluorobenzyl)-1H-imidazol-4-yl)amino)-1-oxopropan-2-yl)-4,4-difluoropiperidin-3-yl)pyridine 1-oxide

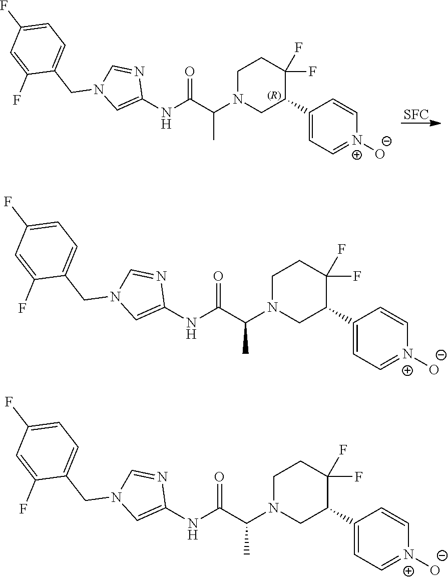

4-((3R)-1-(1-((1-(2,4-difluorobenzyl)-1H-imidazol-4-yl)amino)-1-oxopropan-2-yl)-4,4-difluoropiperidin-3-yl)pyridine 1-oxide (50 mg) was purified by Chiral HPLC with the following conditions (Column: CHIRALPAK IC, 2×25 cm, 5 μm; Mobile Phase A: MtBE (10 mM NH3-MeOH), Mobile Phase B: MEOH; Flow rate: 20 mL/min; Gradient: isocratic 50% B; Wave Length: 268/214 nm; RT1 (min): 6.82; RT2 (min): 13.98; Sample Solvent: MeOH) to afford 4-((R)-1-((S)-1-((1-(2,4-difluorobenzyl)-1H-imidazol-4-yl)amino)-1-oxopropan-2-yl)-4,4-difluoropiperidin-3-yl)pyridine 1-oxide (Compound 3) (Method B, 1.09 min, peak 1, 16.1 mg, 100% e.e.) as a white solid and 4-((R)-1-((R)-1-((1-(2,4-difluorobenzyl)-1H-imidazol-4-yl)amino)-1-oxopropan-2-yl)-4,4-difluoropiperidin-3-yl)pyridine 1-oxide (Compound 4) (Method B, 2.21 min, peak 2, 6.2 mg, 100% e.e.) as a white solid.

TABLE 2

| Compound# | Characterization Data |
|---|---|
| 3 | LCMS (ESI) [M + H]⁺: 478.2 |
| | ¹H NMR (400 MHz, Methanol-d₄) δ 8.27 (d, J = 6.8 Hz, 2H), 7.62-7.48 (m, 3H), 7.46- |
| | 7.34 (m, 1H), 7.27 (d, J = 1.6 Hz, 1H), 7.08-6.93 (m, 2H), 5.21 (s, 2H), 3.62 (ddt, |
| | J = 26.1, 11.1, 4.2 Hz, 1H), 3.49 (q, J = 7.0 Hz, 1H), 3.02-2.95 (m, 2H), 2.88-2.76 |
| | (m, 2H), 2.39-2.08 (m, 2H), 1.31 (d, J = 7.0 Hz, 3H). |
| 4 | LCMS (ESI) [M + H]⁺: 478.2 |
| | ¹H NMR (400 MHz, Methanol-d₄) δ 8.27 (d, J = 6.8 Hz), 7.57 (d, J =7.2 Hz, 3H), 7.38 |
| | (td, J = 8.6, 6.3 Hz, 1H), 7.28 (d, J = 1.6 Hz, 1H), 7.08-6.92 (m, 2H), 5.22 (s, 2H), |
| | 3.67-3.53 (m, 1H), 3.49 (q, J = 7.0 Hz, 1H), 3.01 (d, J = 9.2 Hz, 2H), 2.96-2.90 (m, |
| | 1H), 2.62 (td, J = 11.7, 3.3 Hz, 1H), 2.36-2.12 (m, 2H), 1.31 (d, J = 7.0 Hz, 3H). |

### Example 2: 4-((3S)-1-(1-((1-(3,5-difluorobenzyl)-1H-imidazol-4-yl)amino)-1-oxopropan-2-yl)-4,4-difluoropiperidin-3-yl)pyridine 1-oxide

### Step 1: 4-iodo-1-trityl-1H-imidazole

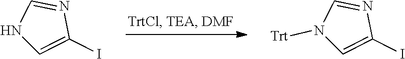

To a mixture of 4-iodo-1H-imidazole (5 g, 25.7 mmol) and triethylamine (7.83 g, 77.3 mmol) in DMF (20 mL) was added triphenylmethyl chloride (7.19 g, 25.7 mmol) in portions at 0° C. under a nitrogen atmosphere. The resulting mixture was stirred at 25° C. for 2 hours. The mixture was poured into ice water and filtered. The filter cake was collected and air dried to afford 4-iodo-1-trityl-1H-imidazole (9 g, 80.03% yield) as a white solid. LCMS (ESI) [M+H]⁺: 437.

### Step 2: 2-hydroxy-N-(1-trityl-1H-imidazol-4-yl)propanamide

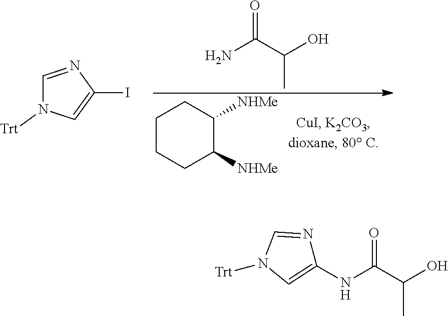

A mixture of 4-iodo-1-trityl-1H-imidazole (7 g, 16.044 mmol), lactamide (1.43 g, 16.044 mmol), copper iodide (0.31 g, 1.604 mmol), K₂CO₃ (6.65 g, 48.132 mmol, 3 equiv) and (1S,2S)—N,N′-Dimethyl-1,2-cyclohexanediamine (0.23 g, 1.604 mmol, 0.1 equiv) in dioxane (40 mL) was stirred for 2 hours at 80° C. under a nitrogen atmosphere. The resulting mixture was concentrated under vacuum. The crude residue was loaded onto a silica gel column and eluted with DCM/MeOH (98:2). This yielded 2-hydroxy-N-(1-trityl-1H-imidazol-4-yl)propanamide (4.05 g, 63.51% yield) as a yellow oil. LCMS (ESI) [M+H]⁺: 398.

### Step 3: 1-oxo-1-((1-trityl-1H-imidazol-4-yl)amino)propan-2-yl 4-methylbenzenesulfonate

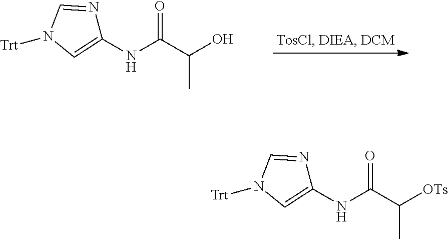

To a mixture of 2-hydroxy-N-(1-trityl-1H-imidazol-4-yl)propanamide (1.2 g, 3.019 mmol) and DIEA (1.17 g, 9.057 mmol) in DCM (15 mL) was added tosyl chloride (1.15 g, 6.038 mmol) in portions at 0° C. under a nitrogen atmosphere. The resulting mixture was stirred at 25° C. for 24 hours. Upon completion of the reaction, the mixture was concentrated under reduced pressure and the crude residue was purified by silica gel column chromatography, eluted with DCM/MeOH (10:1) to afford 1-oxo-1-((1-trityl-1H-imidazol-4-yl)amino)propan-2-yl 4-methylbenzenesulfonate (600 mg, 36.03% yield) as a yellow oil. LCMS (ESI) [M+H]⁺:552.

### Step 4: 4-((3S)-4,4-difluoro-1-(1-oxo-1-((1-trityl-1H-imidazol-4-yl)amino)propan-2-yl)piperidin-3-yl)pyridine 1-oxide

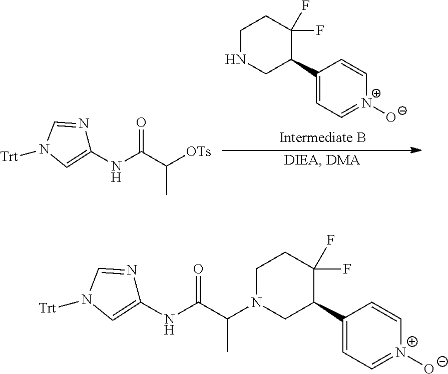

Into a 8-mL sample vial was placed 1-oxo-1-((1-trityl-1H-imidazol-4-yl)amino)propan-2-yl 4-methylbenzenesulfonate (150 mg, 0.272 mmol), Intermediate B (58.25 mg, 0.272 mmol), Hunig's Base (96 μL) and DMA (2 mL). The mixture was stirred at 50° C. for 5 hours until LCMS suggested the completion of the reaction by complete consumption of the starting material. The resulting solution was allowed to cool down to room temperature, poured into water (10 mL) and extracted with EA (3*10 mL). Organic layers were combined, dried over MgSO₄, filtered and concentrated. The crude residue was purified by silica gel chromatography (methanol:dichloromethane=1:15) to give 4-((3S)-4,4-difluoro-1-(1-oxo-1-((1-trityl-1H-imidazol-4-yl)amino)propan-2-yl)piperidin-3-yl)pyridine 1-oxide (130 mg, 80.53%) as a yellow oil. LCMS (ESI) [M+H]⁺: 594.

### Step 5: 4-((3S)-1-(1-((1H-imidazol-4-yl)amino)-1-oxopropan-2-yl)-4,4-difluoropiperidin-3-yl)pyridine 1-oxide

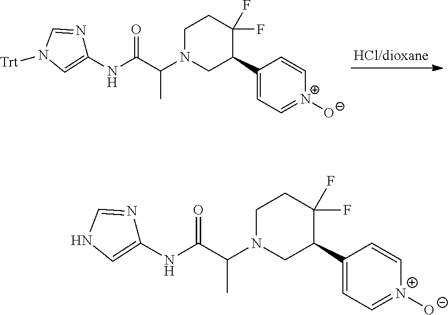

HCl (3 mL, 6 mmol, 2M in dioxane) was added into a 8-mL sample vial containing 4-((3S)-4,4-difluoro-1-(1-oxo-1-((1-trityl-1H-imidazol-4-yl)amino)propan-2-yl)piperidin-3-yl)pyridine 1-oxide (130 mg, 0.219 mmol). The mixture was stirred at 25° C. for 3 hours until LCMS suggested the completion of the reaction by complete consumption of the starting material. The mixture was concentrated and the crude residue was purified by silica gel chromatography (ethyl acetate:petroleum ether=1:2 methanol:dichloromethane=1:15) to give 4-((3S)-1-(1-((1H-imidazol-4-yl)amino)-1-oxopropan-2-yl)-4,4-difluoropiperidin-3-yl)pyridine 1-oxide (70 mg, 91%) as yellow oil. LCMS (ESI) [M+H]⁺: 352.

### Step 6: 4-((3S)-1-(1-((1-(3,5-difluorobenzyl)-1H-imidazol-4-yl)amino)-1-oxopropan-2-yl)-4,4-difluoropiperidin-3-yl)pyridine 1-oxide

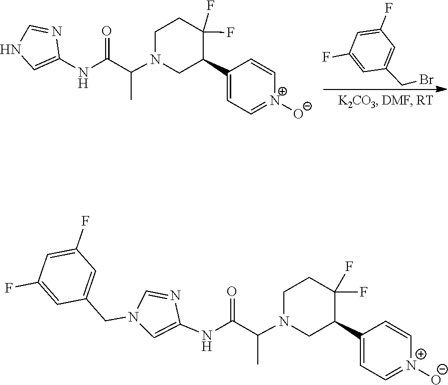 [SMILES: CC(C(=O)Nc1c[nH]cn1)N1CCC(F)(F)[C@@H](c2cc[n+]([O-])cc2)C1 ; Fc1cc(F)cc(CBr)c1 ; CC(C(=O)Nc1cn(Cc2cc(F)cc(F)c2)cn1)N1CCC(F)(F)[C@@H](c2cc[n+]([O-])cc2)C1]

In a 8-mL sample vial was placed 4-((3S)-1-(1-((1H-imidazol-4-yl)amino)-1-oxopropan-2-yl)-4,4-difluoropiperidin-3-yl)pyridine 1-oxide (70 mg, 0.199 mmol), 1-(bromomethyl)-3,5-difluorobenzene (41.24 mg, 0.199 mmol), K₂CO₃ (82.60 mg, 0.598 mmol) and 5 mL DMF. The mixture was stirred at 25° C. for 5 hours until LCMS suggested the completion of the reaction. The mixture was directly purified by Prep-HPLC [Mobile Phase A: water (0.1% NH₄HCO₃), Mobile Phase B: acetonitrile; Gradient: 50% B to 80% B in 8 min; Rt: 6.03 min] to give 4-((3S)-1-(1-((1-(3,5-difluorobenzyl)-1H-imidazol-4-yl)amino)-1-oxopropan-2-yl)-4,4-difluoropiperidin-3-yl)pyridine 1-oxide (35 mg, 36.79%) as a yellow oil. LCMS (ESI)[M+H]⁺: 478.

### Step 7: 4-((S)-1-((S)-1-((1-(3,5-difluorobenzyl)-1H-imidazol-4-yl)amino)-1-oxopropan-2-yl)-4,4-difluoropiperidin-3-yl)pyridine 1-oxide and 4-((S)-1-((R)-1-((1-(3,5-difluorobenzyl)-1H-imidazol-4-yl)amino)-1-oxopropan-2-yl)-4,4-difluoropiperidin-3-yl)pyridine 1-oxide

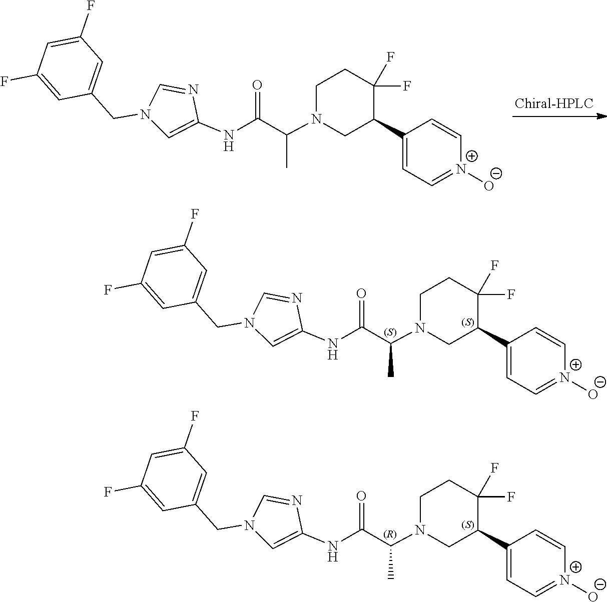

4-((3S)-1-(1-((1-(3,5-difluorobenzyl)-1H-imidazol-4-yl)amino)-1-oxopropan-2-yl)-4,4-difluoropiperidin-3-yl)pyridine 1-oxide (35 mg) was separated by prep-Chiral-HPLC (Column: CHIRALPAK IC, 3×25 cm, 5 μm; Mobile Phase A: MtBE (10 mM NH₃-MeOH), Mobile Phase B: MeOH; Flow rate: 40 mL/min; Gradient: isocratic 50% B; Wave Length: 212/268 nm; RT1 (min): 6.5; RT2 (min): 11.3) to afford 4-((S)-1-((S)-1-((1-(3,5-difluorobenzyl)-1H-imidazol-4-yl)amino)-1-oxopropan-2-yl)-4,4-difluoropiperidin-3-yl)pyridine 1-oxide (Compound 6) (Method C, 0.79 min, peak 1, 16.0 mg, 100% e.e.) and 4-((S)-1-((R)-1-((1-(3,5-difluorobenzyl)-1H-imidazol-4-yl)amino)-1-oxopropan-2-yl)-4,4-difluoropiperidin-3-yl)pyridine 1-oxide (Compound 5) (Method C, 1.99 min, peak 2, 15.0 mg, 100% e.e.).

TABLE 3

| Compound# | Characterization Data |
|---|---|
| 5 | LCMS (ESI) [M + H]⁺: 478.2 |
| | ¹H NMR (400 MHz, Methanol-d₄) δ 8.45 (s, 1H), 8.32 (d, J = 7.1 Hz, 2H), 7.57 (d, J = 6.7 |
| | Hz, 2H), 7.45 (d, J = 1.9 Hz, 1H), 7.04-6.94 (m, 3H), 5.35 (s, 2H), 3.78-3.61 (m, 2H), |
| | 3.25-3.17 (m, 2H), 3.16-3.09 (m, 1H), 2.86-2.80 (m, 1H), 2.34-2.28 (m, 2H), 1.42 (d, |
| | J = 7.0 Hz, 3H). |
| 6 | LCMS (ESI) [M + H]⁺: 478.2 |
| | ¹H NMR (400 MHz, Methanol-d₄) δ 8.50 (s, 1H), 8.32 (m, 2H), 7.57 (d, J = 6.8Hz, 2H), |
| | 7.49 (s, 1H), 7.03-6.97 (m, 3H), 5.35 (s, 2H), 3.85-3.56 (m, 2H), 3.28-2.97 (m, 4H), |
| | 2.46-2.21 (m, 2H), 1.45-1.43 (m, 3H). |

### Example 3: 4-((3S)-1-(1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide

### Step 1: 4-nitro-2-(trifluoromethyl)-1H-imidazole

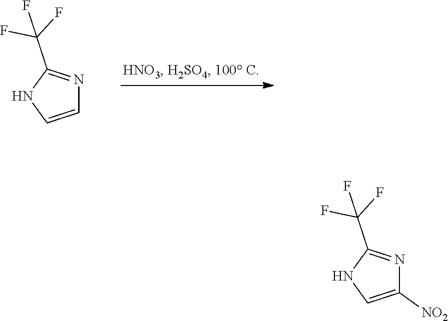 [SMILES: FC(F)(F)c1ncc[nH]1 ; O=[N+]([O-])c1c[nH]c(C(F)(F)F)n1]

2-(trifluoromethyl)-1H-imidazole (1.0 g, 7.35 mmol) was slowly added into a 20 mL sample vial containing HNO₃ (3 mL, 98% in H₂O) and H₂SO₄ (3 mL, 98% in H₂O). The resulting mixture was heated and stirred for 1 hour at 100° C. Upon completion of the reaction as monitored by TLC. The mixture was cooled to room temperature and slowly poured into ice water (40 mL). The white precipitate was collected by filtration to afford crude 4-nitro-2-(trifluoromethyl)-1H-imidazole (1.5 g) after air-dry. This crude was further purified by flash column chromatography (PE/EA=1:1) to afford 4-nitro-2-(trifluoromethyl)-1H-imidazole (800 mg, 61.5% yield) as a white solid. LCMS (ESI) [M+H]⁺: 182.

### Step 2: 1-(2,4-difluorobenzyl)-4-nitro-2-(trifluoromethyl)-1H-imidazole

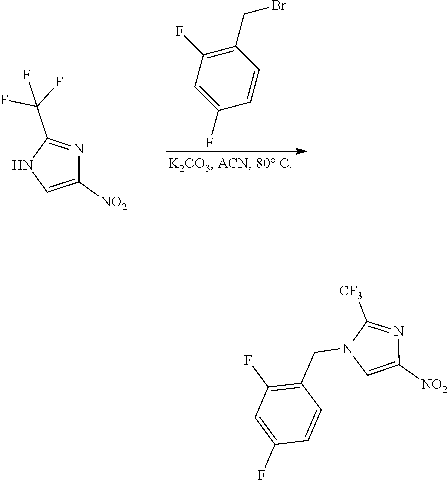 [SMILES: O=[N+]([O-])c1c[nH]c(C(F)(F)F)n1 ; Fc1ccc(CBr)c(F)c1 ; O=[N+]([O-])c1cn(Cc2ccc(F)cc2F)c(C(F)(F)F)n1]

A mixture of 4-nitro-2-(trifluoromethyl)-1H-imidazole (500 mg, 2.76 mmol), 1-(bromomethyl)-2,4-difluorobenzene (686 mg, 3.31 mmol) and potassium carbonate (762 mg, 5.52 mmol) in acetonitrile (5 mL) was stirred at 80° C. for 3 hours until completion by LCMS. The resulting mixture was filtered and the filter cake was washed with DCM (3×15 mL). The filtrate was concentrated under reduced pressure and the crude product was purified by flash column chromatography (PE/EA=3:2) to yield 1-(2,4-difluorobenzyl)-4-nitro-2-(trifluoromethyl)-1H-imidazole (300 mg, 35.4% yield). LCMS (ESI) [M+H]⁺: 308.

### Step 3: 1-(2,4-difluorobenzyl)-2-(trifluoromethyl)-1H-imidazol-4-amine

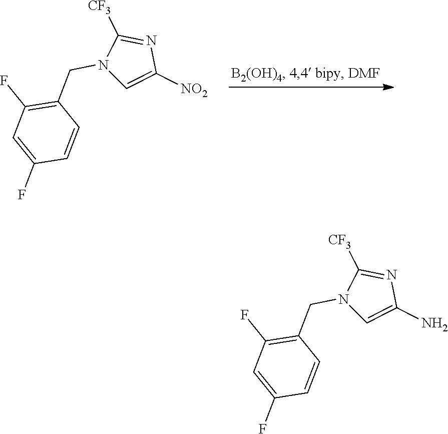 [SMILES: O=[N+]([O-])c1cn(Cc2ccc(F)cc2F)c(C(F)(F)F)n1 ; Nc1cn(Cc2ccc(F)cc2F)c(C(F)(F)F)n1]

A solution of tetrahydroxydiboron (292 mg, 3.25 mmol) in DMF (2 mL) was slowly added into a solution of 1-(2,4-difluorobenzyl)-4-nitro-2-(trifluoromethyl)-1H-imidazole (200 mg, 651.2 μmol) and 4, 4′ bipyridine (10 mg, 65.1 μmol) in DMF (2 mL) at 0° C. Upon completion of the addition, the solution turned purple and was allowed to warm back to room temperature. The solution was stirred for another 30 minutes and became colorless again. TLC suggested a complete consumption of the starting material. The mixture was poured into water (20 mL) and extracted with EA (3×20 mL). Organic layers were separated, combined, dried over MgSO₄, filtered and concentrated under vacuum to give crude 1-(2,4-difluorobenzyl)-2-(trifluoromethyl)-1H-imidazol-4-amine (180 mg, 99.4% quant), which was used for next step directly without further purification.

### Step 4: 2-bromo-N-(1-(2,4-difluorobenzyl)-2-(trifluoromethyl)-1H-imidazol-4-yl)propanamide

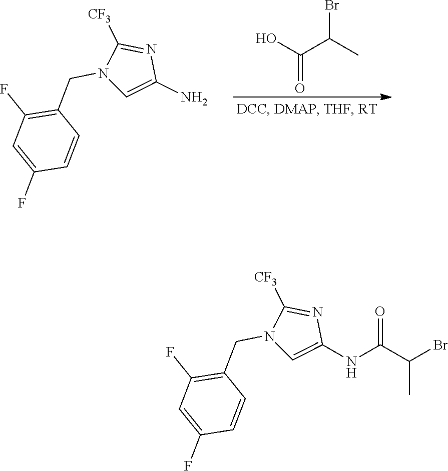

A mixture of 1-(2,4-difluorobenzyl)-2-(trifluoromethyl)-1H-imidazol-4-amine (400 mg, 649.36 μmol), 2-bromopropanoic acid (298 mg, 1.95 mmol), DCC (473 mg, 2.29 mmol) and DMAP (65 mg, 64.9 μmol) in THF (5 mL) was stirred for overnight at room temperature. LCMS suggested completely consumption of the starting material. Upon completion, the resulting mixture was concentrated under reduced pressure and purified by silica gel column chromatography, eluted with DCM/MeOH (95:5) to afford 2-bromo-N-(1-(2,4-difluorobenzyl)-2-(trifluoromethyl)-1H-imidazol-4-yl)propanamide (120 mg, 44.8% yield) as a yellow oil.

LCMS (ESI) [M+H]⁺: 412.

### Step 5: 4-((3S)-1-(1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide

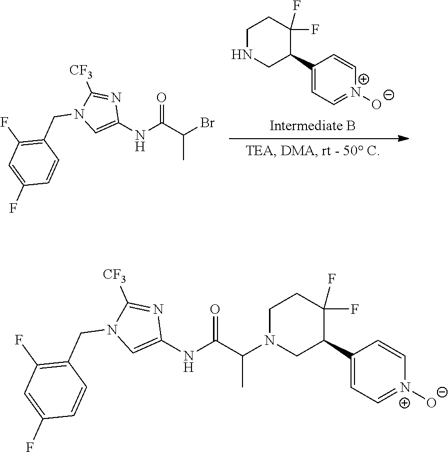 [SMILES: CC(Br)C(=O)Nc1cn(Cc2ccc(F)cc2F)c(C(F)(F)F)n1 ; [O-][n+]1ccc([C@H]2CNCCC2(F)F)cc1 ; CC(C(=O)Nc1cn(Cc2ccc(F)cc2F)c(C(F)(F)F)n1)N1CCC(F)(F)[C@@H](c2cc[n+]([O-])cc2)C1]

A mixture of 2-bromo-N-(1-(2,4-difluorobenzyl)-2-(trifluoromethyl)-1H-imidazol-4-yl)propanamide (100 mg, 0.29 mmol), (S)-4-(4,4-difluoropiperidin-3-yl)pyridine 1-oxide (Intermediate B) (52 mg, 0.24 mmol) and TEA (74 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide (55.0 mg, 41.7% yield) as a white solid. LCMS (ESI) [M+H]⁺: 546.

### Step 6: 4-((S)-1-((S)-1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide and 4-((S)-1-((R)-1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide

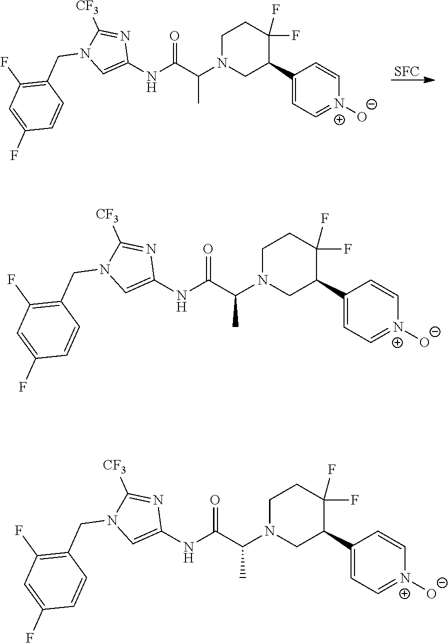

4-((3S)-1-(1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide (55 mg) was separated by Prep-Chiral HPLC with the following conditions Column: CHIRALPAK IE, 3×25 cm, 5 μm; Mobile Phase A: HEX:MtBE=1:1 (1:1 (0.5% 2M NH3-MEOH), Mobile Phase B: MeOH; Flow rate: 40 mL/min; Gradient: isocratic 15% B; Wave Length: 217/270 nm; RT1 (min): 17.2; RT2 (min): 23.6; Sample Solvent: MeOH:DCM=8:1; Injection Volume: 1 mL; Number Of Runs: 2 to afford 4-((S)-1-((S)-1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide (Compound 7) (Method D, 2.02 min, peak 1, 9.0 mg, 100% e.e.) as a white solid and 4-((S)-1-((R)-1-((1-(2,4-difluorobenzyl)-2-(trifluoromethyl)-1H-imidazol-4-yl)amino)-1-oxopropan-2-yl)-4,4-difluoropiperidin-3-yl)pyridine 1-oxide (Compound 8) (Method D, 2.82 min, peak 2, 6.0 mg, 100% e.e.) as a white solid.

TABLE 4

| Compound# | Characterization Data |
|---|---|
| 7 | LCMS (ESI) [M + H]⁺: 546.1. |
| | ¹H NMR (400 MHz, Methanol-d₄) δ 8.29 (d, J = 7.1 Hz, 2H), 7.57 (d, J = 6.7 Hz, 2H), 7.48 |
| | (s, 1H), 7.39-7.18 (m, 1H), 7.11-6.88 (m, 2H), 5.38 (s, 2H), 3.72-3.54 (m, 1H), 3.51 (q, |
| | J = 7.0 Hz, 1H), 3.08-2.84 (m, 3H), 2.70-2.58 (m, 1H), 2.37-2.11 (m, 2H), 1.31 (d, J = |
| | 7.0 Hz, 3H). |
| | ¹⁹F NMR (377 MHz, Methanol-d₄) δ −62.44 (s, 3F, CF₃), −98.22 (d, J = 238.7 Hz, 1 |
| | F), −107.63-−118.36 (m, 1 F). |
| 8 | LCMS (ESI) [M + H]⁺: 546.2. |
| | ¹H NMR (400 MHz, Methanol-d₄) δ 8.28 (d, J = 7.0 Hz, 2H), 7.56 (d, J = 6.6 Hz, 2H), 7.48 |
| | (s, 1H), 7.32-7.21 (m, 1H), 7.11-6.87 (m, 2H), 5.38 (s, 2H), 3.65-3.60 (m, 1H), 3.50 (g, |
| | J = 6.8 Hz, 1H), 3.04-2.92 (m, 2H), 2.88-2.75 (m, 2H), 2.38-2.12 (m, 2H), 1.31 (d, J = 7.0 |
| | Hz, 3H). |
| | ¹⁹F NMR (377 MHz, Methanol-d₄) δ −62.44 (d, J = 3.0 Hz, 3F, CF₃), −98.20 (d, J = 238.7 Hz, |
| | 1 F), −108.79-−116.48 (m, 1 F). |

### Example 4: 4-((3S)-1-(1-((1-(2,4-difluorobenzyl)-2-methyl-1H-imidazol-4-yl)amino)-1-oxopropan-2-yl)-4,4-difluoropiperidin-3-yl)pyridine 1-oxide

### Step 1: 1-(2,4-difluorobenzyl)-2-methyl-4-nitro-1H-imidazole

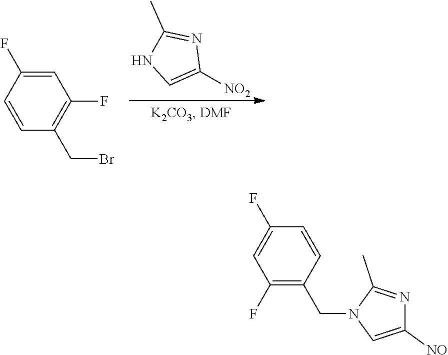

A solution of 1-(bromomethyl)-2,4-difluorobenzene (500 mg, 2.41 mmol), 2-methyl-4-nitro-1H-imidazole (368 mg, 2.89 mmol) and K₂CO₃ (1 g, 7.23 mmol) in DMF (5 mL) was stirred for 1 hour at room temperature. The reaction was quenched with water. The resulting mixture was extracted with EtOAc (3×25 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford 1-(2,4-difluorobenzyl)-2-methyl-4-nitro-1H-imidazole (550 mg, 89% yield) as a white solid. LCMS (ESI) [M+H]⁺: 254.

### Step 2: 1-(2,4-difluorobenzyl)-2-methyl-1H-imidazol-4-amine

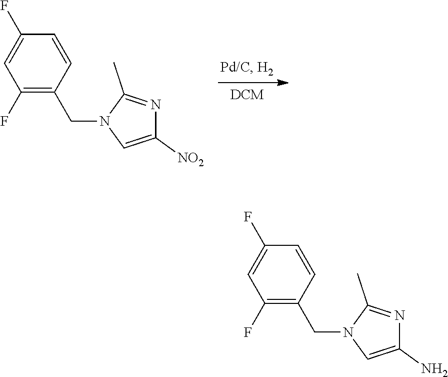 [SMILES: Cc1nc([N+](=O)[O-])cn1Cc1ccc(F)cc1F ; Cc1nc(N)cn1Cc1ccc(F)cc1F]

A solution of 1-(2,4-difluorobenzyl)-2-methyl-4-nitro-1H-imidazole (550 mg, 2.17 mmol) and 10% Pd/C (55 mg, anhydrous) in DCM (5 mL) was stirred for 2 hours at room temperature under hydrogen atmosphere (2 atm). The resulting mixture was filtered, and the filter cake was washed with DCM (3×10 mL). The filtrate was concentrated under reduced pressure to afford 1-(2,4-difluorobenzyl)-2-methyl-1H-imidazol-4-amine (450 mg, crude) as a white solid which was used with no further purification. LCMS (ESI) [M+H]⁺: 224.

### Step 3: 2-bromo-N-(1-(2,4-difluorobenzyl)-2-methyl-1H-imidazol-4-yl)propanamide

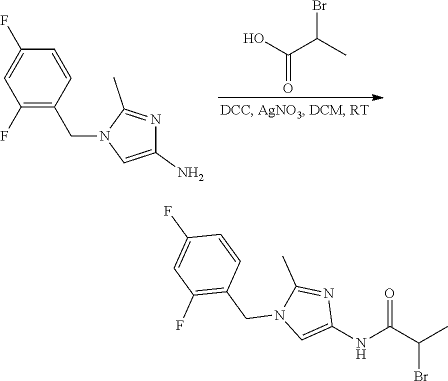

A mixture of 1-(2,4-difluorobenzyl)-2-methyl-1H-imidazol-4-amine (450 mg, 2.01 mmol), 2-bromopropanoic acid (370 mg, 2.41 mmol), DCC (499 mg, 2.41 mmol) and AgNO₃ (68 mg, 0.40 mmol) in DCM (10 mL) was stirred for overnight at room temperature under air atmosphere. The resulting mixture was filtered, and the filter cake was washed with DCM (3×10 mL). The filtrate was concentrated under reduced pressure. The residue was purified by silica gel column chromatography, eluted with PE/EA (1:1) to afford 2-bromo-N-(1-(2,4-difluorobenzyl)-2-methyl-1H-imidazol-4-yl)propanamide (350 mg, 48.47% yield) as a white solid. LCMS (ESI) [M+H]⁺: 358.

### Step 4: 4-((3S)-1-(1-((1-(2,4-difluorobenzyl)-2-methyl-1H-imidazol-4-yl)amino)-1-oxopropan-2-yl)-4,4-difluoropiperidin-3-yl)pyridine 1-oxide

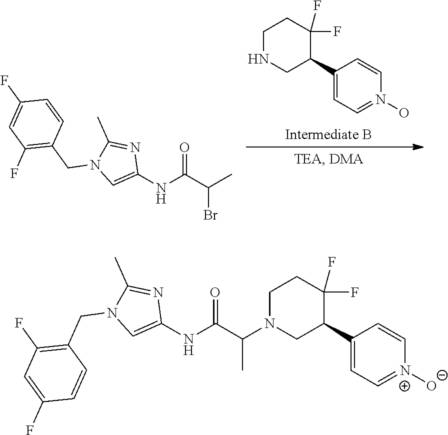

A mixture of 2-bromo-N-(1-(2,4-difluorobenzyl)-2-methyl-1H-imidazol-4-yl)propanamide (100 mg, 0.27 mmol), (S)-4-(4,4-difluoropiperidin-3-yl)pyridine 1-oxide (Intermediate B) (71 mg, 0.33 mmol) and TEA (85 mg, 0.84 mmol) in DMA (3 mL) was stirred for 2 days at room temperature. The resulting mixture was purified by Prep-HPLC with the following conditions (Column: Xselect CSH Prep C18 OBD Column, 30×150 mm, 5 μm; Mobile Phase A: Water (0.1% FA), Mobile Phase B: ACN; Flow rate: 60 mL/min; Gradient: 10% B to 30% B in 10 min; Wave Length: 254 nm/220 nm; RT1 (min): 12.28) to afford 4-((3S)-1-(1-((1-(2,4-difluorobenzyl)-2-methyl-1H-imidazol-4-yl)amino)-1-oxopropan-2-yl)-4,4-difluoropiperidin-3-yl)pyridine 1-oxide (80 mg, 58% yield) as a white solid. LCMS (ESI) [M+H]⁺: 492

### Step 5: 4-((S)-1-((S)-1-((1-(2,4-difluorobenzyl)-2-methyl-1H-imidazol-4-yl)amino)-1-oxopropan-2-yl)-4,4-difluoropiperidin-3-yl)pyridine 1-oxide and 4-((S)-1-((R)-1-((1-(2,4-difluorobenzyl)-2-methyl-1H-imidazol-4-yl)amino)-1-oxopropan-2-yl)-4,4-difluoropiperidin-3-yl)pyridine 1-oxide

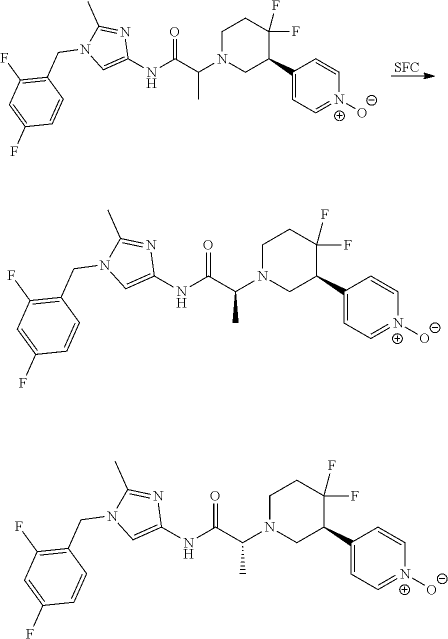

4-((3S)-1-(1-((1-(2,4-difluorobenzyl)-2-methyl-1H-imidazol-4-yl)amino)-1-oxopropan-2-yl)-4,4-difluoropiperidin-3-yl)pyridine 1-oxide (80 mg) was purified by Prep-Chiral HPLC with the following conditions (Column: CHIRALPAK IE, 3×25 cm, 5 μm; Mobile Phase A: MtBE (10 mM NH3-MeOH), Mobile Phase B: MEOH; Flow rate: 40 mL/min; Gradient: isocratic 40% B; Wave Length: 268/214 nm; RT1 (min): 6.2; RT2 (min): 7.58; Sample Solvent: MEOH; Injection Volume: 1.0 mL; Number Of Runs: 3) to afford 4-((S)-1-((S)-1-((1-(2,4-difluorobenzyl)-2-methyl-1H-imidazol-4-yl)amino)-1-oxopropan-2-yl)-4,4-difluoropiperidin-3-yl)pyridine 1-oxide (Compound 9) (Method E, 0.75 min, peak 1, 27.9 mg, 100% e.e.) as a white solid and 4-((S)-1-((R)-1-((1-(2,4-difluorobenzyl)-2-methyl-1H-imidazol-4-yl)amino)-1-oxopropan-2-yl)-4,4-difluoropiperidin-3-yl)pyridine 1-oxide (Compound 10) (Method E, 0.94 min, peak 2, 14 mg, 100% e.e.) as a white solid.

TABLE 5

| Compound# | Characterization Data |
|---|---|
| 9 | LCMS (ESI) [M + H]⁺: 492.2 |
| | ¹H NMR (400 MHz, DMSO-d₆) δ 10.07 (s, 1H), 8.17 (d, J = 7.2 Hz, 2H), 7.37 (d, J = 6.7 |
| | Hz, 2H), 7.34-7.23 (m, 2H), 7.12 (d, J = 4.9 Hz, 2H), 5.12 (s, 2H), 3.58-3.39 (m, 3H), |
| | 3.02-2.80 (m, 3H), 2.27 (s, 3H), 2.12-2.03 (m, 2H), 1.16 (d, J = 6.9 Hz, 3H). |
| 10 | LCMS (ESI) [M + H]⁺: 492.2 |
| | ¹H NMR (400 MHz, DMSO-d₆) δ 10.07 (s, 1H), 8.15 (d, J = 7.2 Hz, 2H), 7.37-7.26 (m, |
| | 4H), 7.14-7.09 (m, 2H), 5.11 (s, 2H), 3.50-3.39 (m, 2H), 2.94-2.90 (m, 2H), 2.69- |
| | 2.61 (m, 2H), 2.26 (s, 3H), 2.10-1.90 (m, 2H), 1.17 (d, J = 6.9 Hz, 3H). |

### Example 5: 4-((3S)-1-(1-([1,3]dioxolo[4′,5′:4,5]benzo[1,2-d]thiazol-6-ylamino)-1-oxopropan-2-yl)-4,4-difluoropiperidin-3-yl)pyridine 1-oxide

### Step 1: N-([1,3]dioxolo[4′,5′:4,5]benzo[1,2-d]thiazol-6-yl)-2-bromopropanamide

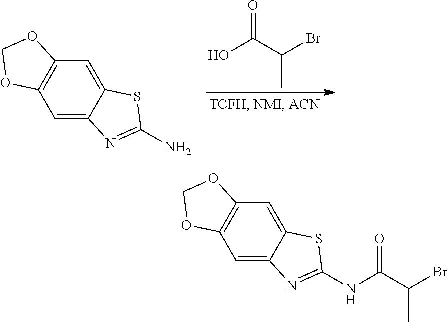

A mixture of [1,3]dioxolo[4′,5′:4,5]benzo[1,2-d]thiazol-6-amine (70 mg, 360.44 μmol), 2-bromopropanoic acid (55.14 mg, 360.44 μmol), NMI (45 mg, 540.66 μmol) and TCFH (227 mg, 540.66 μmol) in ACN (2 mL) was stirred for overnight at room temperature. LCMS suggested complete consumption of the starting material. The resulting mixture was directly purified by reverse phase column chromatography, eluted with formic acid (0.1 M in H₂O) to afford N-([1,3]dioxolo[4′,5′:4,5]benzo[1,2-d]thiazol-6-yl)-2-bromopropanamide (75 mg, 63.5% yield) as a white solid. LCMS (ESI) [M+H]⁺: 329.15/331.15.

### Step 2: 4-((3S)-1-(1-([1,3]dioxolo[4′,5′:4,5]benzo[1,2-d]thiazol-6-ylamino)-1-oxopropan-2-yl)-4,4-difluoropiperidin-3-yl)pyridine 1-oxide

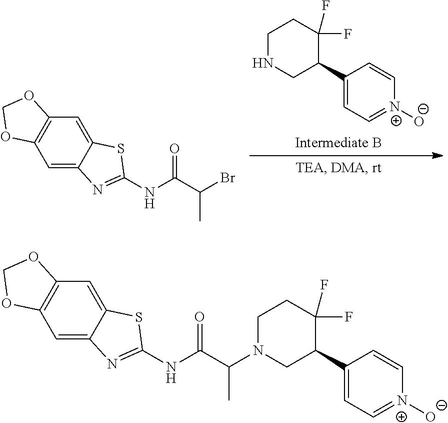 [SMILES: CC(Br)C(=O)Nc1nc2cc3c(cc2s1)OCO3 ; [O-][n+]1ccc([C@H]2CNCCC2(F)F)cc1 ; CC(C(=O)Nc1nc2cc3c(cc2s1)OCO3)N1CCC(F)(F)[C@@H](c2cc[n+]([O-])cc2)C1]

A mixture of N-([1,3]dioxolo[4′,5′:4,5]benzo[1,2-d]thiazol-6-yl)-2-bromopropanamide (75 mg, 227.85 μmol), (S)-4-(4,4-difluoropiperidin-3-yl)pyridine 1-oxide (Intermediate B) (49 mg, 227.85 μmol) and TEA (70 mg, 684.2 μmol) in DMA (1 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to 4-((3S)-1-(1-([1,3]dioxolo[4′,5′:4,5]benzo[1,2-d]thiazol-6-ylamino)-1-oxopropan-2-yl)-4,4-difluoropiperidin-3-yl)pyridine 1-oxide (46.0 mg, 43.8% yield) as a white solid. LCMS (ESI) [M+H]⁺: 463.12.

### Step 3: 4-((S)-1-((S)-1-([1,3]dioxolo[4′,5′:4,5]benzo[1,2-d]thiazol-6-ylamino)-1-oxopropan-2-yl)-4,4-difluoropiperidin-3-yl)pyridine 1-oxide and 4-((S)-1-((S)-1-([1,3]dioxolo[4′,5′:4,5]benzo[1,2-d]thiazol-6-ylamino)-1-oxopropan-2-yl)-4,4-difluoropiperidin-3-yl)pyridine 1-oxide

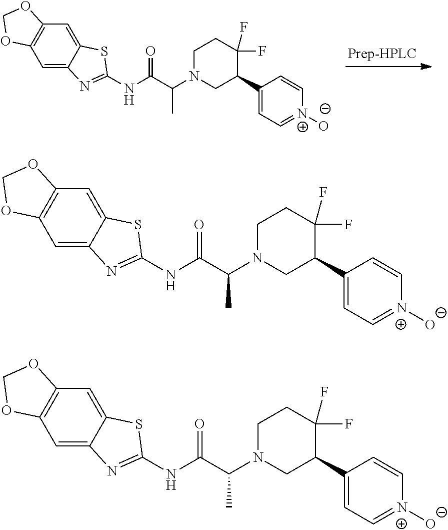 [SMILES: CC(C(=O)Nc1nc2cc3c(cc2s1)OCO3)N1CCC(F)(F)[C@@H](c2cc[n+]([O-])cc2)C1 ; C[C@@H](C(=O)Nc1nc2cc3c(cc2s1)OCO3)N1CCC(F)(F)[C@@H](c2cc[n+]([O-])cc2)C1 ; C[C@H](C(=O)Nc1nc2cc3c(cc2s1)OCO3)N1CCC(F)(F)[C@@H](c2cc[n+]([O-])cc2)C1]

4-((3S)-1-(1-([1,3]dioxolo[4′,5′:4,5]benzo[1,2-d]thiazol-6-ylamino)-1-oxopropan-2-yl)-4,4-difluoropiperidin-3-yl)pyridine 1-oxide (46 mg) was separated by Prep-HPLC with the following conditions Column: XSelect CSH Fluoro Phenyl 30×150 mm, 5 μm; Mobile Phase A: Water (0.1% FA), Mobile Phase B: ACN; Flow rate: 60 mL/min; Gradient: 19% B to 35% B in 9 min; Wave Length: 254 nm/220 nm; RT1 (min): 12.15/12.77 to afford first peak 4-((S)-1-((S)-1-([1,3]dioxolo[4′,5′:4,5]benzo[1,2-d]thiazol-6-ylamino)-1-oxopropan-2-yl)-4,4-difluoropiperidin-3-yl)pyridine 1-oxide (Compound 12) (Method F, 4.08 min, peak 1, 15.0 mg, 96.64% e.e.) as a white solid and second peak 4-((S)-1-((S)-1-([1,3]dioxolo[4′,5′:4,5]benzo[1,2-d]thiazol-6-ylamino)-1-oxopropan-2-yl)-4,4-difluoropiperidin-3-yl)pyridine 1-oxide (Compound 11) (Method F, 4.52 min, peak 2, 9.0 mg, 100% e.e.) as a white solid.

TABLE 6

| Compound# | Characterization Data |
|---|---|
| 11 | LCMS (ESI) [M + H]⁺: 463.12. |
| | ¹H NMR (400 MHz, Methanol-d₄) δ 8.30 (d, J = 7.0 Hz, 2H), 7.60 (d, J = 6.6 Hz, 2H), 7.30 |
| | (s, 1H), 7.20 (s, 1H), 6.02 (s, 2H), 3.71 (q, J = 7.0 Hz, 1H), 3.65-3.55 (m, 1H), 3.13-3.07 |
| | (m, 2H), 2.96-2.88 (m, 1H), 2.78-2.63 (m, 1H), 2.43-2.07 (m, 2H), 1.38 (d, J = 6.9 Hz, 3H). |
| | ¹⁹F NMR (377 MHz, Methanol-d₄) δ −98.13 (d, J = 239.2 Hz, 2F, CF₂). |
| 12 | LCMS (ESI) [M + H]⁺: 463.12. |
| | ¹H NMR (400 MHz, Methanol-d⁴) δ 8.27 (d, J = 7.1 Hz, 2H), 7.57 (d, J = 6.7 Hz, 2H), 7.29 |
| | (s, 1H), 7.19 (s, 1H), 6.01 (s, 2H), 3.69 (q, J = 7.0 Hz, 1H), 3.63-3.59 (m, 1H), 3.09-2.95 |
| | (m, 2H), 2.94-2.82 (m, 2H), 2.41-2.11 (m, 2H), 1.38 (d, J = 6.9 Hz, 3H). |
| | ¹⁹F NMR (377 MHz, Methanol-d₄) δ −98.17 (d, J = 239.1 Hz, 2F, CF₂). |

### Example 6: 4-((3S)-1-(1-((1-(1-(3,5-difluorophenyl)ethyl)-1H-imidazol-4-yl)amino)-1-oxopropan-2-yl)-4,4-difluoropiperidin-3-yl)pyridine 1-oxide

### Step 1: 1-(3,5-difluorophenyl)ethan-1-ol

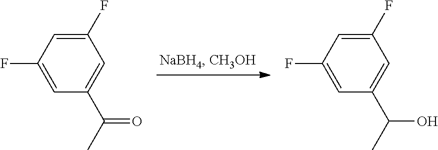

A mixture of 1-(3,5-difluorophenyl)ethan-1-one (2 g, 12.81 mmol, 1 equiv) and sodium boron hydride (1.45 g, 39 mmol, 3 equiv) in methanol (10 mL) was stirred for 5 h at room temperature. The resulting mixture was concentrated under reduced pressure. The residue was purified by silica gel column chromatography, eluted with PE/EA (7:3) to afford 1-(3,5-difluorophenyl)ethan-1-ol (1.5 g, 74% yield) as a white solid.

### Step 2: 1-(1-bromoethyl)-3,5-difluorobenzene

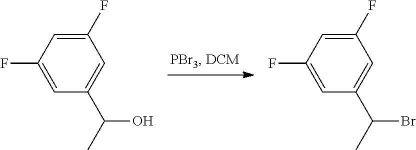

A mixture of 1-(3,5-difluorophenyl)ethan-1-ol (1.5 g, 9.48 mmol, 1 equiv) and PBr₃ (2.56 g, 9.48 mmol, 1 equiv) in DCM (10 mL) was stirred for 2 h at room temperature. The resulting mixture was concentrated under reduced pressure. The residue was purified by silica gel column chromatography, eluted with PE to afford 1-(1-bromoethyl)-3,5-difluorobenzene (1 g, 47% yield) as light yellow oil.

### Step 3: 1-(1-(3,5-difluorophenyl)ethyl)-4-nitro-1H-imidazole

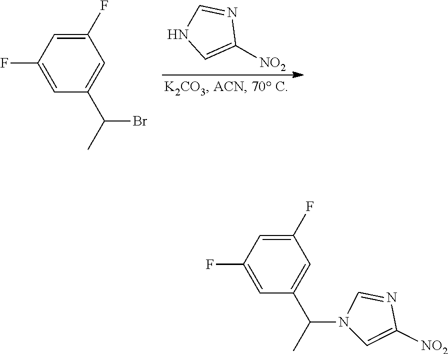 [SMILES: CC(Br)c1cc(F)cc(F)c1 ; O=[N+]([O-])c1c[nH]cn1 ; CC(c1cc(F)cc(F)c1)n1cnc([N+](=O)[O-])c1]

A mixture of 4-nitro-1H-imidazole (511 mg, 4.52 mmol, 1 equiv), 1-(1-bromoethyl)-3,5-difluorobenzene (1 g, 4.52 mmol, 1 equiv), K₂CO₃ (1.6 g, 13 mmol, 3 equiv) in ACN (10 mL) was stirred for 2 h at 80° C. The resulting mixture was concentrated under reduced pressure. The residue was purified by silica gel column chromatography, eluted with CH₂Cl₂/PE (9:1) to afford 1-(1-(3,5-difluorophenyl)ethyl)-4-nitro-1H-imidazole (1 g, 87% yield) as a white solid.

LCMS (ESI) [M+H]⁺: 254.07.

### Step 4: 1-(1-(3,5-difluorophenyl)ethyl)-1H-imidazol-4-amine

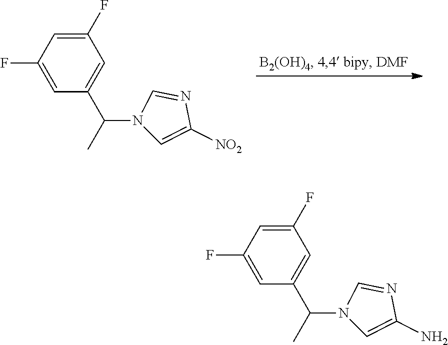 [SMILES: CC(c1cc(F)cc(F)c1)n1cnc([N+](=O)[O-])c1 ; CC(c1cc(F)cc(F)c1)n1cnc(N)c1]

A solution of 1-(1-(3,5-difluorophenyl)ethyl)-4-nitro-1H-imidazole (350 mg, 0.86 mmol, 1 equiv) and tetrahydroxydiboron (166 mg, 3.3 mmol, 4 equiv) in DMF (3 mL) was added 4-(pyridin-4-yl)pyridine (10 mg, 0.38 mmol, 0.3 equiv) dropwise at 0° C. The resulting mixture was stirred at 0° C. for 15 minutes. Upon completion, the mixture was diluted with water (10 mL). The aqueous layer was extracted with EtOAc (3×20 mL). The resulting mixture was concentrated under reduced pressure. This resulted in 1-(1-(3,5-difluorophenyl)ethyl)-1H-imidazol-4-amine (248 mg, 80% yield) as a white solid, which was used for next step directly without further purification.

LCMS (ESI) [M+H]⁺: 224.09.

### Step 5: 2-bromo-N-(1-(1-(3,5-difluorophenyl)ethyl)-1H-imidazol-4-yl)propanamide

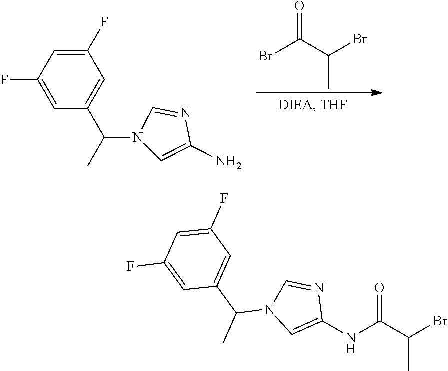

A solution of 1-(1-(3,5-difluorophenyl)ethyl)-1H-imidazol-4-amine (248 mg, 1.1 mmol, 1 equiv), DIEA (500 mg, 3.6 mmol, 3 equiv) and 2-bromopropanoyl bromide (280 mg, 1.1 mmol, 1 equiv) in THF (8 mL) was stirred for 30 min at room temperature. The resulting mixture was concentrated under reduced pressure. The residue was purified by silica gel column chromatography, eluted with PE/EA (5:1) to afford 2-bromo-N-(1-(1-(3,5-difluorophenyl)ethyl)-1H-imidazol-4-yl)propanamide (159 mg, 39% yield) as a white solid.

LCMS (ESI) [M+H]⁺: 358.03

### Step 6: 4-((3S)-1-(1-((1-(1-(3,5-difluorophenyl)ethyl)-1H-imidazol-4-yl)amino)-1-oxopropan-2-yl)-4,4-difluoropiperidin-3-yl)pyridine 1-oxide

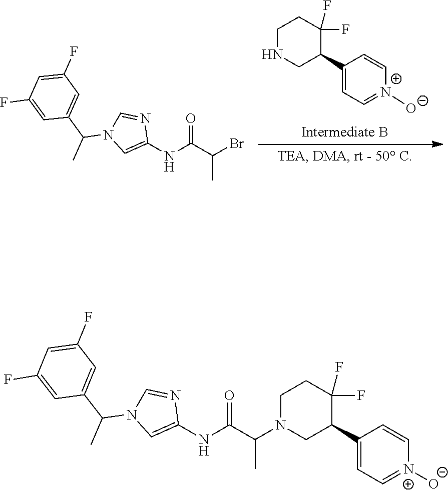 [SMILES: CC(Br)C(=O)Nc1cn(C(C)c2cc(F)cc(F)c2)cn1 ; [O-][n+]1ccc([C@H]2CNCCC2(F)F)cc1 ; CC(C(=O)Nc1cn(C(C)c2cc(F)cc(F)c2)cn1)N1CCC(F)(F)[C@@H](c2cc[n+]([O-])cc2)C1]

A mixture of 2-bromo-N-(1-(1-(3,5-difluorophenyl)ethyl)-1H-imidazol-4-yl)propanamide (159 mg, 0.44 mmol), (S)-4-(4,4-difluoropiperidin-3-yl)pyridine 1-oxide (Intermediate B) (95 mg, 0.44 mmol) and TEA (100 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-((1-(1-(3,5-difluorophenyl)ethyl)-1H-imidazol-4-yl)amino)-1-oxopropan-2-yl)-4,4-difluoropiperidin-3-yl)pyridine 1-oxide (58.0 mg, 26% yield) as a white solid.

LCMS (ESI) [M+H]⁺: 492.19.

### Step 7: 4-((S)-1-((S)-1-((1-((S)-1-(3,5-difluorophenyl)ethyl)-1H-imidazol-4-yl)amino)-1-oxopropan-2-yl)-4,4-difluoropiperidin-3-yl)pyridine 1-oxide, 4-((S)-1-((R)-1-((1-((R)-1-(3,5-difluorophenyl)ethyl)-1H-imidazol-4-yl)amino)-1-oxopropan-2-yl)-4,4-difluoropiperidin-3-yl)pyridine 1-oxide, 4-((S)-1-((R)-1-((1-((S)-1-(3,5-difluorophenyl)ethyl)-1H-imidazol-4-yl)amino)-1-oxopropan-2-yl)-4,4-difluoropiperidin-3-yl)pyridine 1-oxide and 4-((S)-1-((S)-1-((1-((R)-1-(3,5-difluorophenyl)ethyl)-1H-imidazol-4-yl)amino)-1-oxopropan-2-yl)-4,4-difluoropiperidin-3-yl)pyridine 1-oxide

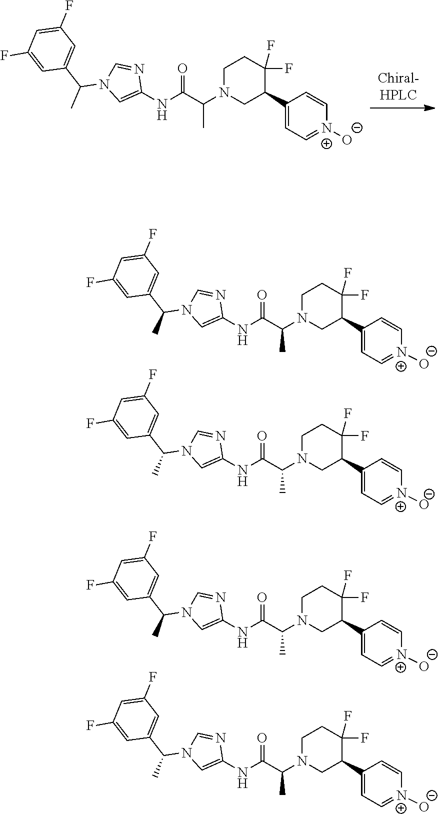 [SMILES: CC(C(=O)Nc1cn(C(C)c2cc(F)cc(F)c2)cn1)N1CCC(F)(F)[C@@H](c2cc[n+]([O-])cc2)C1 ; C[C@@H](C(=O)Nc1cn([C@@H](C)c2cc(F)cc(F)c2)cn1)N1CCC(F)(F)[C@@H](c2cc[n+]([O-])cc2)C1 ; C[C@H](C(=O)Nc1cn([C@H](C)c2cc(F)cc(F)c2)cn1)N1CCC(F)(F)[C@@H](c2cc[n+]([O-])cc2)C1 ; C[C@H](C(=O)Nc1cn([C@@H](C)c2cc(F)cc(F)c2)cn1)N1CCC(F)(F)[C@@H](c2cc[n+]([O-])cc2)C1 ; C[C@H](c1cc(F)cc(F)c1)n1cnc(NC(=O)[C@H](C)N2CCC(F)(F)[C@@H](c3cc[n+]([O-])cc3)C2)c1]

4-((3S)-1-(1-((1-(1-(3,5-difluorophenyl)ethyl)-1H-imidazol-4-yl)amino)-1-oxopropan-2-yl)-4,4-difluoropiperidin-3-yl)pyridine 1-oxide (58.0 mg) was separated by reversed-phase flash chromatography with the following conditions: column, C18; mobile phase, MeCN in water (10 mmol/L NH₄HCO₃), 5% to 100% gradient in 20 min to give two portions (24 mg, RT: 11 min, 27 mg, RT: 14 min), each is a mixture of diastereomers. The first portion was separated by Prep-Chiral HPLC with the following conditions Column: CHIRALPAK IA, 3×25 cm, 5 μm; Mobile Phase A: MtBE (10 mM NH₃-MeOH), Mobile Phase B: EtOH; Flow rate: 40 mL/min; Gradient: isocratic 50% B; Wave Length: 212/268 nm; RT1 (min): 5.89; RT2 (min): 10.08; Sample Solvent: MEOH; Injection Volume: 2.6 mL; Number Of Runs: 2 to afford 4-((S)-1-((R)-1-((1-((S)-1-(3,5-difluorophenyl)ethyl)-1H-imidazol-4-yl)amino)-1-oxopropan-2-yl)-4,4-difluoropiperidin-3-yl)pyridine 1-oxide (Compound 13) (Method G, 1.07 min, peak 1, 6.9 mg, 100% e.e.) as a white solid (RT1) and 4-((S)-1-((S)-1-((1-((R)-1-(3,5-difluorophenyl)ethyl)-1H-imidazol-4-yl)amino)-1-oxopropan-2-yl)-4,4-difluoropiperidin-3-yl)pyridine 1-oxide (Compound 14) (Method G, 2.45 min, peak 2, 8.6 mg, 98.94% e.e.) (RT2). The second portion was separated by Column: CHIRALPAK IE, 3×25 cm, 5 μm; Mobile Phase A: MtBE (10 mM NH₃-MeOH), Mobile Phase B: MEOH; Flow rate: 40 mL/min; Gradient: isocratic 20% B; Wave Length: 214/272 nm; RT1 (min): 9.87; RT2 (min): 16.12; Sample Solvent: MeOH:DCM=1:1; Injection Volume: 2.0 mL; Number Of Runs: 2 to afford 4-((S)-1-((S)-1-((1-((S)-1-(3,5-difluorophenyl)ethyl)-1H-imidazol-4-yl)amino)-1-oxopropan-2-yl)-4,4-difluoropiperidin-3-yl)pyridine 1-oxide (Compound 15) (Method H, 1.26 min, peak 1, 8.7 mg, 100% e.e.) as a white solid (RT1) and 4-((S)-1-((R)-1-((1-((R)-1-(3,5-difluorophenyl)ethyl)-1H-imidazol-4-yl)amino)-1-oxopropan-2-yl)-4,4-difluoropiperidin-3-yl)pyridine 1-oxide (Compound 16) (Method H, 1.59 min, peak 2, 7.5 mg, 100% e.e.) (RT2) as a white solid.

TABLE 7

| Compound# | Characterization Data |
|---|---|
| 13 | LCMS (ESI) [M + H]⁺: 492.1. |
| | ¹H NMR (400 MHz, Methanol-d₄) δ 8.29 (d, J = 7.1 Hz, 2H), 7.68 (d, J = 1.6 Hz, 1H), 7.57 (d, |
| | J = 6.6 Hz, 2H), 7.33 (d, J = 1.6 Hz, 1H), 6.98-6.81 (m, 3H), 5.51 (q, J = 7.1 Hz, 1H), 3.68-3.41 |
| | (m, 3H), 3.16-2.85 (m, 3H), 2.63 (td, J = 11.5, 3.1 Hz, 1H), 2.47-2.08 (m, 1H), 1.85 (d, J = |
| | 7.0 Hz, 3H), 1.32 (d, J = 7.0 Hz, 3H). |
| 14 | LCMS (ESI) [M + H]⁺: 492.1. |
| | ¹H NMR (400 MHz, Methanol-d₄) δ 8.29 (d, J = 7.0 Hz, 2H), 7.68 (s, 1H), 7.57 (d, J = 6.5 Hz, |
| | 2H), 7.33 (s, 1H), 6.98-6.81 (m, 3H), 5.51 (q, J = 7.0 Hz, 1H), 3.69-3.40 (m, 2H), 3.15-2.89 |
| | (m, 3H), 2.75-2.55 (m, 1H), 2.41-2.10 (m, 2H), 1.85 (d, J = 7.1 Hz, 3H), 1.32 (d, J = 7.0 Hz, 3H). |
| 15 | LCMS (ESI) [M + H]⁺: 492.2. |
| | ¹H NMR (400 MHz, Methanol-d₄) δ 8.28 (d, J = 7.0 Hz, 2H), 7.68 (d, J = 1.6 Hz, 1H), 7.57 (d, |
| | J = 6.7 Hz, 2H), 7.33 (d, J = 1.6 Hz, 1H), 6.98-6.81 (m, 3H), 5.51 (q, J = 7.1 Hz, 1H), 3.80-3.40 |
| | (m, 2H), 3.09-2.69 (m, 4H), 2.40-2.08 (m, 2H), 1.85 (d, J = 7.1 Hz, 3H), 1.32 (d, J = 7.0 Hz, 3H). |
| 16 | LCMS (ESI) [M + H]⁺: 492.2. |
| | ¹H NMR (400 MHz, Methanol-d₄) δ 8.28 (d, J = 7.1 Hz, 2H), 7.68 (d, J = 1.6 Hz, 1H), 7.57 (d, |
| | J = 6.8 Hz, 2H), 7.33 (d, J = 1.6 Hz, 1H), 6.95-6.71 (m, 3H), 5.50 (q, J = 7.0 Hz, 1H), 3.72-3.42 |
| | (m, 2H), 3.13-2.69 (m, 4H), 2.41-2.08 (m, 2H), 1.85 (d, J = 7.1 Hz, 3H), 1.32 (d, J = 7.0 Hz, 3H). |

### Example 7: 4-((3S)-1-(1-((1-(3-cyano-5-fluorobenzyl)-2-methyl-1H-imidazol-4-yl)amino)-1-oxopropan-2-yl)-4,4-difluoropiperidin-3-yl)pyridine 1-oxide

### Step 1: 3-(bromomethyl)-5-fluorobenzonitrile

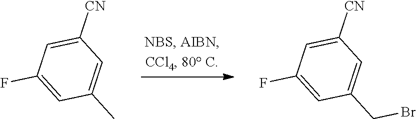

A mixture of 3-fluoro-5-methylbenzonitrile (500 mg, 3.7 mmol, 1 equiv), AIBN (728 mg, 4.44 mmol, 1.2 equiv), NBS (790 mg, 4.44 mmol, 1.2 equiv) in CCl₄ (5 mL) was stirred for 2 h at 80° C. The resulting mixture was concentrated under reduced pressure. The residue was purified by silica gel column chromatography, eluted with PE to afford 3-(bromomethyl)-5-fluorobenzonitrile (320 mg, 40% yield) as light yellow oil.

### Step 2: 3-fluoro-5-((2-methyl-4-nitro-1H-imidazol-1-yl)methyl)benzonitrile

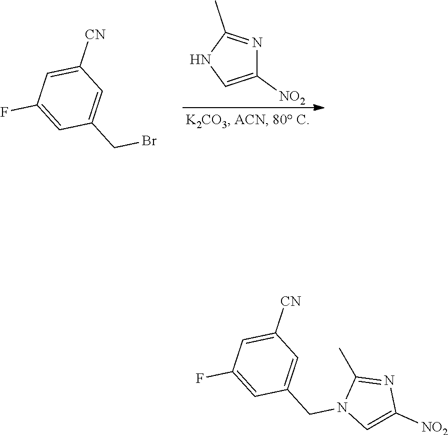

A mixture of 2-methyl-4-nitro-1H-imidazole (190 mg, 1.5 mmol, 1 equiv), 3-(bromomethyl)-5-fluorobenzonitrile (320 mg, 1.5 mmol, 1 equiv), K₂CO₃ (621 mg, 4.5 mmol, 3 equiv) in ACN (5 mL) was stirred for 2 h at 80° C. The resulting mixture was concentrated under reduced pressure. The residue was purified by silica gel column chromatography, eluted with CH₂Cl₂/PE (9:1) to afford 3-fluoro-5-((2-methyl-4-nitro-1H-imidazol-1-yl)methyl)benzonitrile (224 mg, 57% yield) as a white solid.

LCMS (ESI) [M+H]⁺: 261.07.

### Step 3: 3-((4-amino-2-methyl-1H-imidazol-1-yl)methyl)-5-fluorobenzonitrile

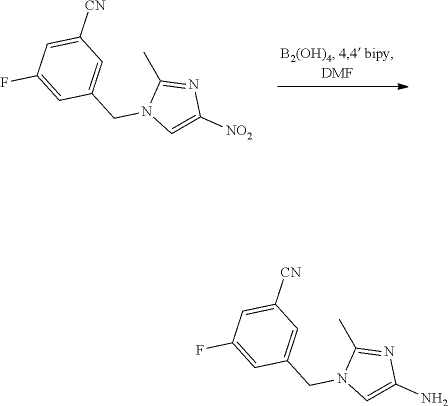 [SMILES: Cc1nc([N+](=O)[O-])cn1Cc1cc(F)cc(C#N)c1 ; Cc1nc(N)cn1Cc1cc(F)cc(C#N)c1]

A solution of 3-fluoro-5-((2-methyl-4-nitro-1H-imidazol-1-yl)methyl)benzonitrile (224 mg, 0.86 mmol, 1 equiv) and tetrahydroxydiboron (166 mg, 3.3 mmol, 4 equiv) in DMF (3 mL) was added 4-(pyridin-4-yl)pyridine (10 mg, 0.38 mmol, 0.3 equiv) dropwise at 0° C. The resulting mixture was stirred at 0° C. for 15 minutes. Upon completion, the mixture was diluted with water (10 mL). The aqueous layer was extracted with EtOAc (3×20 mL). The resulting mixture was concentrated under reduced pressure. This resulted in 3-((4-amino-2-methyl-1H-imidazol-1-yl)methyl)-5-fluorobenzonitrile (150 mg, 75% yield) as a white solid, which was used for next step directly without further purification.

LCMS (ESI) [M+H]⁺: 231.10.

### Step 4: 2-bromo-N-(1-(3-cyano-5-fluorobenzyl)-2-methyl-1H-imidazol-4-yl)propanamide

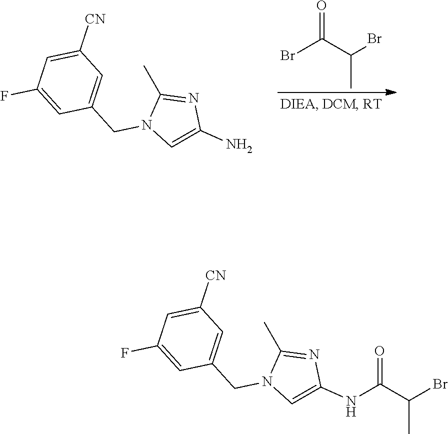

A solution of 3-((4-amino-2-methyl-1H-imidazol-1-yl)methyl)-5-fluorobenzonitrile (150 mg, 0.65 mmol, 1 equiv), DIEA (250 mg, 1.8 mmol, 3 equiv) and 2-bromopropanoyl bromide (140 mg, 0.65 mmol, 1 equiv) in DCM (5 mL) was stirred for 30 min at room temperature. The resulting mixture was concentrated under reduced pressure. The residue was purified by silica gel column chromatography, eluted with PE/EA (5:1) to afford 2-bromo-N-(1-(3-cyano-5-fluorobenzyl)-2-methyl-1H-imidazol-4-yl)propanamide (144 mg, 60% yield) as a white solid.

LCMS (ESI) [M+H]⁺: 365

### Step 5: 4-((3S)-1-(1-((1-(3-cyano-5-fluorobenzyl)-2-methyl-1H-imidazol-4-yl)amino)-1-oxopropan-2-yl)-4,4-difluoropiperidin-3-yl)pyridine 1-oxide

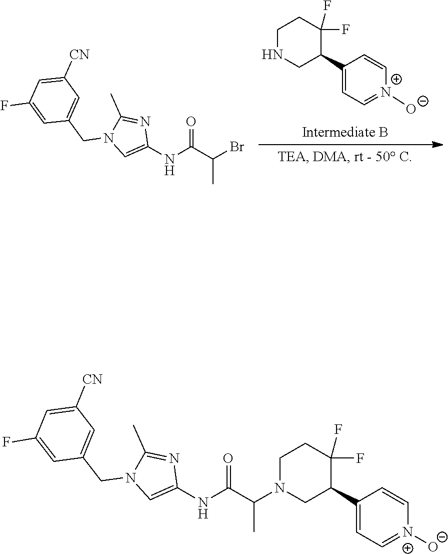 [SMILES: Cc1nc(NC(=O)C(C)Br)cn1Cc1cc(F)cc(C#N)c1 ; [O-][n+]1ccc([C@H]2CNCCC2(F)F)cc1 ; Cc1nc(NC(=O)C(C)N2CCC(F)(F)[C@@H](c3cc[n+]([O-])cc3)C2)cn1Cc1cc(F)cc(C#N)c1]

A mixture of 2-bromo-N-(1-(3-cyano-5-fluorobenzyl)-2-methyl-1H-imidazol-4-yl)propanamide (144 mg, 0.39 mmol), (S)-4-(4,4-difluoropiperidin-3-yl)pyridine 1-oxide (Intermediate B) (84 mg, 0.39 mmol) and TEA (100 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-((1-(3-cyano-5-fluorobenzyl)-2-methyl-1H-imidazol-4-yl)amino)-1-oxopropan-2-yl)-4,4-difluoropiperidin-3-yl)pyridine 1-oxide (86.7 mg, 44% yield) as a white solid.

LCMS (ESI) [M+H]⁺: 499.20.

### Step 6: 4-((S)-1-((S)-1-((1-(3-cyano-5-fluorobenzyl)-2-methyl-1H-imidazol-4-yl)amino)-1-oxopropan-2-yl)-4,4-difluoropiperidin-3-yl)pyridine 1-oxide and 4-((S)-1-((R)-1-((1-(3-cyano-5-fluorobenzyl)-2-methyl-1H-imidazol-4-yl)amino)-1-oxopropan-2-yl)-4,4-difluoropiperidin-3-yl)pyridine 1-oxide

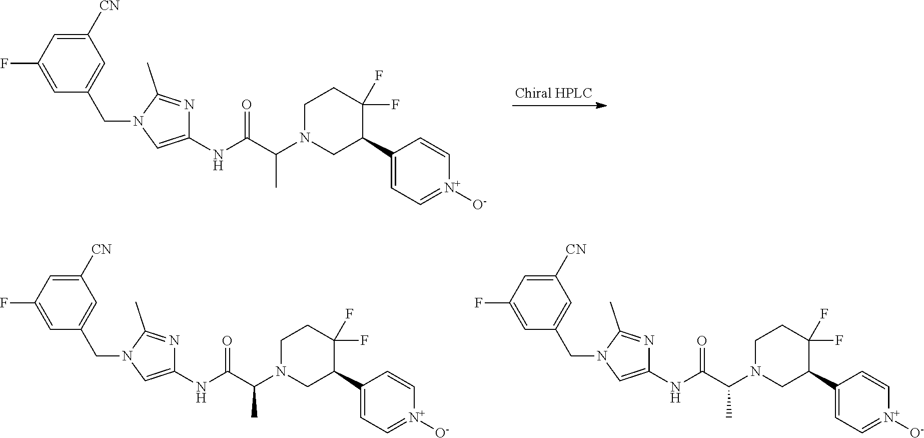 [SMILES: Cc1nc(NC(=O)C(C)N2CCC(F)(F)[C@@H](c3cc[n+]([O-])cc3)C2)cn1Cc1cc(F)cc(C#N)c1 ; Cc1nc(NC(=O)[C@H](C)N2CCC(F)(F)[C@@H](c3cc[n+]([O-])cc3)C2)cn1Cc1cc(F)cc(C#N)c1 ; Cc1nc(NC(=O)[C@@H](C)N2CCC(F)(F)[C@@H](c3cc[n+]([O-])cc3)C2)cn1Cc1cc(F)cc(C#N)c1]

4-((3S)-1-(1-((1-(3-cyano-5-fluorobenzyl)-2-methyl-1H-imidazol-4-yl)amino)-1-oxopropan-2-yl)-4,4-difluoropiperidin-3-yl)pyridine 1-oxide (86.7 mg) was separated by Prep-Chiral HPLC with the following conditions Column: CHIRALPAK IE, 3×25 cm, 5 μm; Mobile Phase A: MtBE (10 mM NH₃-MeOH), Mobile Phase B: MEOH; Flow rate: 40 mL/min; Gradient: isocratic 40% B; Wave Length: 212/270 nm; RT1 (min): 7.39; RT2 (min): 8.20; Sample Solvent: MeOH:DCM=1:1; Injection Volume: 0.6 mL; Number Of Runs: 6 to afford 4-((S)-1-((S)-1-((1-(3-cyano-5-fluorobenzyl)-2-methyl-1H-imidazol-4-yl)amino)-1-oxopropan-2-yl)-4,4-difluoropiperidin-3-yl)pyridine 1-oxide (Compound 17) (Method E, 0.75 min, peak 1, 24.9 mg, 100% e.e.) as a white solid and 4-((S)-1-((R)-1-((1-(3-cyano-5-fluorobenzyl)-2-methyl-1H-imidazol-4-yl)amino)-1-oxopropan-2-yl)-4,4-difluoropiperidin-3-yl)pyridine 1-oxide (Compound 18) (Method E, 0.90 min, peak 2, 30.0 mg, 98.94% e.e.) as a white solid.

TABLE 8

| Compound# | Characterization Data |
|---|---|
| 17 | LCMS (ESI) [M + H]⁺: 499.3. |
| | ¹H NMR (400 MHz, Methanol-d₄) δ 8.30 (d, J = 6.8 Hz, 2H), 7.58 (d, J = 6.6 Hz, 2H), 7.52 (dd, |
| | J = 8.1, 2.3 Hz, 1H), 7.37 (s, 1H), 7.28-7.22 (m, 2H), 5.23 (s, 2H), 3.71-3.55 (m, 1H), 3.51 (q, |
| | J = 7.0 Hz, 1H), 3.08-3.00 (m, 2H), 2.98-2.89 (m, 1H), 2.74-2.53 (m, 1H), 2.30 (s, 3H), 2.28- |
| | 2.12 (m, 2H), 1.33 (d, J = 6.9 Hz, 3H). |
| 18 | LCMS (ESI) [M + H]⁺: 499.3. |
| | ¹H NMR (400 MHz, Methanol-d₄) δ 8.29 (d, J = 6.9 Hz, 2H), 7.57 (d, J = 6.6 Hz, 2H), 7.54- |
| | 7.49 (m, 1H), 7.36 (s, 1H), 7.28-7.21 (d, J = 5.8 Hz, 2H), 5.23 (s, 2H), 3.73-3.61 (m, 1H), |
| | 3.51 (q, J = 7.0 Hz, 1H), 3.08-2.91 (m, 2H), 2.89-2.76 (m, 2H), 2.30 (s, 3H), 2.26-2.14 (m, |
| | 2H), 1.33 (d, J = 7.0 Hz, 3H). |

### Example 8: 4-((3S)-1-(1-((1-(3,5-difluorobenzyl)-2-methyl-1H-imidazol-4-yl)amino)-1-oxopropan-2-yl)-4,4-difluoropiperidin-3-yl)pyridine 1-oxide

### Step 1: 1-(3,5-difluorobenzyl)-2-methyl-4-nitro-1H-imidazole

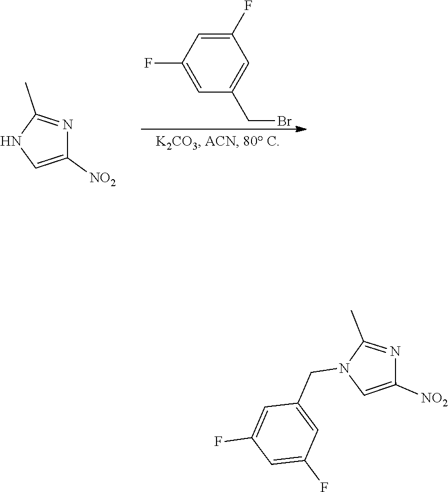

A mixture of 2-methyl-4-nitro-1H-imidazole (700 mg, 5.51 mmol, 1 equiv), 1-(bromomethyl)-3,5-difluorobenzene (1.14 g, 5.51 mmol, 1 equiv), K₂CO₃ (1.60 g, 11.59 mmol, 3 equiv) in ACN (10 mL) was stirred for 2 h at 80° C. The resulting mixture was concentrated under reduced pressure. The residue was purified by silica gel column chromatography, eluted with CH₂Cl₂/PE (9:1) to afford 1-(3,5-difluorobenzyl)-2-methyl-4-nitro-1H-imidazole (630 mg, 53% yield) as a white solid.

LCMS (ESI) [M+H]⁺: 254.10.

### Step 2: 1-(3,5-difluorobenzyl)-2-methyl-1H-imidazol-4-amine

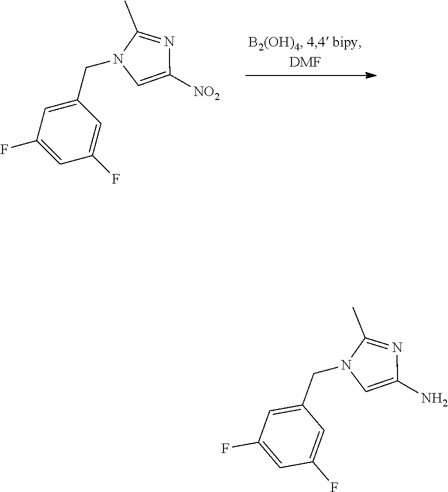 [SMILES: Cc1nc([N+](=O)[O-])cn1Cc1cc(F)cc(F)c1 ; Cc1nc(N)cn1Cc1cc(F)cc(F)c1]

A solution of 1-(3,5-difluorobenzyl)-2-methyl-4-nitro-1H-imidazole (300 mg, 1.18 mmol, 1 equiv) and tetrahydroxydiboron (466 mg, 4.80 mmol, 4 equiv) in DMF (3 mL) was added 4-(pyridin-4-yl)pyridine (60 mg, 0.38 mmol, 0.3 equiv) dropwise at 0° C. The resulting mixture was stirred at 0° C. for 15 minutes. Upon completion, the mixture was diluted with water (10 mL). The aqueous layer was extracted with EtOAc (3×20 mL). The resulting mixture was concentrated under reduced pressure. This resulted in 1-(3,5-difluorobenzyl)-2-methyl-1H-imidazol-4-amine (180 mg, 68% yield) as a white solid, which was used for next step directly without further purification.

LCMS (ESI) [M+H]⁺: 224.09.

### Step 3: 2-bromo-N-(1-(3,5-difluorobenzyl)-2-methyl-1H-imidazol-4-yl)propanamide

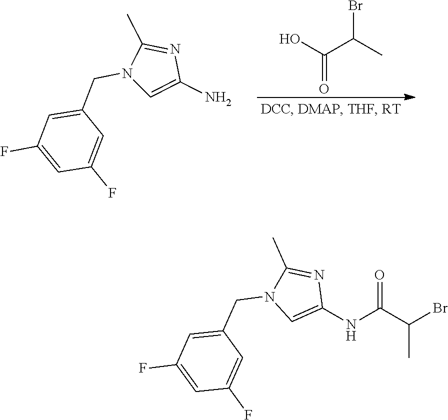

A solution of 1-(3,5-difluorobenzyl)-2-methyl-1H-imidazol-4-amine (180 mg, 0.80 mmol, 1 equiv), DCC (180 mg, 0.95 mmol, 1.2 equiv), DMAP (50 mg, 0.5 mmol, 0.5 equiv) and α-bromopropionic acid (298 mg, 1.94 mmol, 3 equiv) in THF (5 mL) was stirred for 30 min at room temperature. The resulting mixture was concentrated under reduced pressure. The residue was purified by silica gel column chromatography, eluted with PE/EA (5:1) to afford 2-bromo-N-(1-(3,5-difluorobenzyl)-2-methyl-1H-imidazol-4-yl)propanamide (201 mg, 69% yield) as a white solid.

LCMS (ESI) [M+H]⁺: 358

### Step 5: 4-((3S)-1-(1-((1-(3,5-difluorobenzyl)-2-methyl-1H-imidazol-4-yl)amino)-1-oxopropan-2-yl)-4,4-difluoropiperidin-3-yl)pyridine 1-oxide

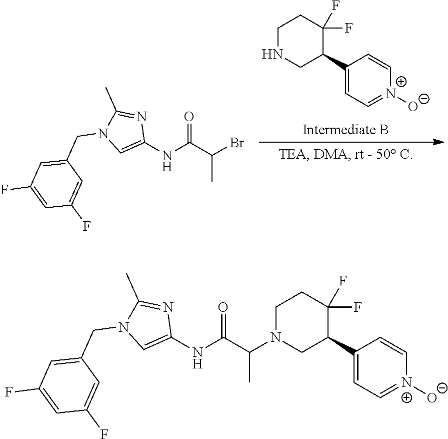 [SMILES: Cc1nc(NC(=O)C(C)Br)cn1Cc1cc(F)cc(F)c1 ; [O-][n+]1ccc([C@H]2CNCCC2(F)F)cc1 ; Cc1nc(NC(=O)C(C)N2CCC(F)(F)[C@@H](c3cc[n+]([O-])cc3)C2)cn1Cc1cc(F)cc(F)c1]

A mixture of 2-bromo-N-(1-(3,5-difluorobenzyl)-2-methyl-1H-imidazol-4-yl)propanamide (100 mg, 0.29 mmol), (S)-4-(4,4-difluoropiperidin-3-yl)pyridine 1-oxide (Intermediate B) (52 mg, 0.24 mmol) and TEA (74 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-((1-(3,5-difluorobenzyl)-2-methyl-1H-imidazol-4-yl)amino)-1-oxopropan-2-yl)-4,4-difluoropiperidin-3-yl)pyridine 1-oxide (33.0 mg, 24% yield) as a white solid. LCMS (ESI) [M+H]⁺: 546.

### Step 6: 4-((S)-1-((S)-1-((1-(3,5-difluorobenzyl)-2-methyl-1H-imidazol-4-yl)amino)-1-oxopropan-2-yl)-4,4-difluoropiperidin-3-yl)pyridine 1-oxide and 4-((S)-1-((R)-1-((1-(3,5-difluorobenzyl)-2-methyl-1H-imidazol-4-yl)amino)-1-oxopropan-2-yl)-4,4-difluoropiperidin-3-yl)pyridine 1-oxide

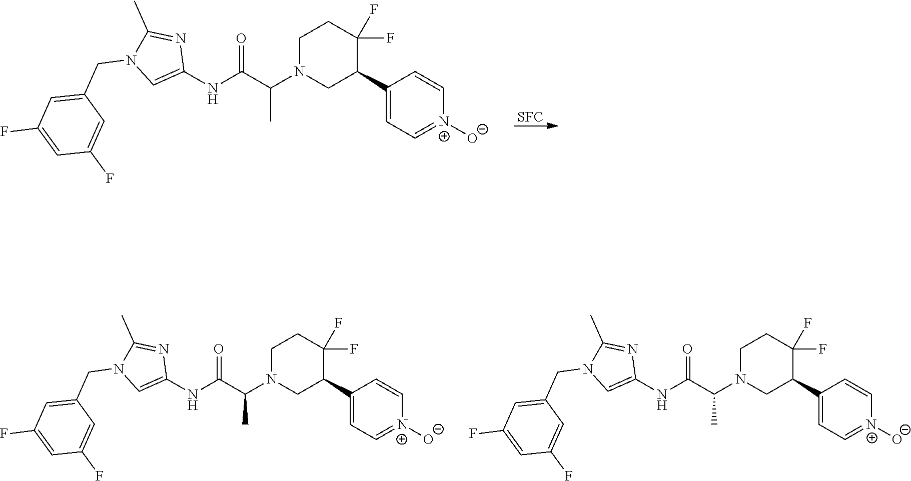

4-((3S)-1-(1-((1-(3,5-difluorobenzyl)-2-methyl-1H-imidazol-4-yl)amino)-1-oxopropan-2-yl)-4,4-difluoropiperidin-3-yl)pyridine 1-oxide (33.0 mg) was separated by Prep-Chiral HPLC with the following conditions Column: CHIRALPAK IE, 3×25 cm, 5 μm; Mobile Phase A: HEX:MtBE=1:1 (1:1 (0.5% 2M NH₃-MEOH), Mobile Phase B: MeOH; Flow rate: 40 mL/min; Gradient: isocratic 15% B; Wave Length: 217/270 nm; RT1 (min): 17.2; RT2 (min): 23.6; Sample Solvent: MeOH:DCM=8:1; Injection Volume: 1 mL; Number Of Runs: 2 to afford 4-((S)-1-((S)-1-((1-(3,5-difluorobenzyl)-2-methyl-1H-imidazol-4-yl)amino)-1-oxopropan-2-yl)-4,4-difluoropiperidin-3-yl)pyridine 1-oxide (Compound 19) (Method E, 0.71 min, peak 1, 10.0 mg, 100% e.e.) as a white solid and 4-((S)-1-((R)-1-((1-(3,5-difluorobenzyl)-2-methyl-1H-imidazol-4-yl)amino)-1-oxopropan-2-yl)-4,4-difluoropiperidin-3-yl)pyridine 1-oxide (Compound 20) (Method E, 0.87 min, peak 2, 11.0 mg, 100% e.e.) as a white solid.

TABLE 9

| Compound# | Characterization Data |
|---|---|
| 19 | LCMS (ESI) [M + H]⁺: 492.1. |
| | ¹H NMR (400 MHz, Methanol-d₄) δ 8.30 (d, J = 6.8 Hz, 2H), 7.58 (d, J = 6.6 Hz, 2H), |
| | 7.23 (s, 1H), 6.89 (dd, J = 10.4, 7.8 Hz, 1H), 6.76 (d, J = 7.1 Hz, 2H), 5.17 (s, 2H), |
| | 3.74-3.54 (m, 1H), 3.51 (q, J = 7.0 Hz, 1H), 3.06-3.01 (m, 2H), 2.98-2.91 (m, |
| | 1H), 2.74-2.58 (m, 1H), 2.29 (s, 3H), 2.38-2.13 (m, 2H), 1.33 (d, J = 6.9 Hz, 3H). |
| 20 | LCMS (ESI) [M + H]⁺: 492.2. |
| | ¹H NMR (400 MHz, Methanol-d₄) δ 8.28 (d, J = 6.8 Hz, 2H), 7.57 (d, J = 6.6 Hz, 2H), |
| | 7.23 (s, 1H), 7.00-6.82 (m, 1H), 6.75 (d, J = 6.9 Hz, 2H), 5.16 (s, 2H), 3.75-3.56 |
| | (m, 1H), 3.51 (q, J = 7.0 Hz, 1H), 3.08-2.90 (m, 2H), 2.89-2.77 (m, 2H), 2.29 (s, |
| | 3H), 2.36-2.14 (m, 2H), 1.33 (d, J = 7.0 Hz, 3H). |

### Example 9: N-(1-(3,5-difluorobenzyl)-2-methyl-1H-imidazol-4-yl)-2-(4,4-difluoropiperidin-1-yl)propanamide

### Step 1: N-(1-(3,5-difluorobenzyl)-2-methyl-1H-imidazol-4-yl)-2-(4,4-difluoropiperidin-1-yl)propanamide

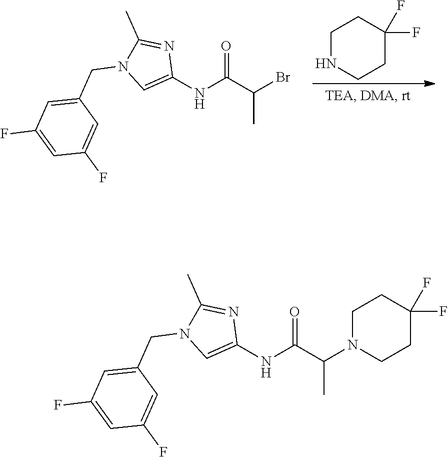 [SMILES: Cc1nc(NC(=O)C(C)Br)cn1Cc1cc(F)cc(F)c1 ; FC1(F)CCNCC1 ; Cc1nc(NC(=O)C(C)N2CCC(F)(F)CC2)cn1Cc1cc(F)cc(F)c1]

A mixture of 2-bromo-N-(1-(3,5-difluorobenzyl)-2-methyl-1H-imidazol-4-yl)propanamide (100 mg, 0.29 mmol), 4,4-difluoropiperidine (78 mg, 0.58 mmol) and TEA (74 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford N-(1-(3,5-difluorobenzyl)-2-methyl-1H-imidazol-4-yl)-2-(4,4-difluoropiperidin-1-yl)propanamide (29.0 mg, 26% yield) as a white solid.

LCMS (ESI) [M+H]⁺: 399.17.

### Step 2: (S)—N-(1-(3,5-difluorobenzyl)-2-methyl-1H-imidazol-4-yl)-2-(4,4-difluoropiperidin-1-yl)propanamide and (R)—N-(1-(3,5-difluorobenzyl)-2-methyl-1H-imidazol-4-yl)-2-(4,4-difluoropiperidin-1-yl)propanamide

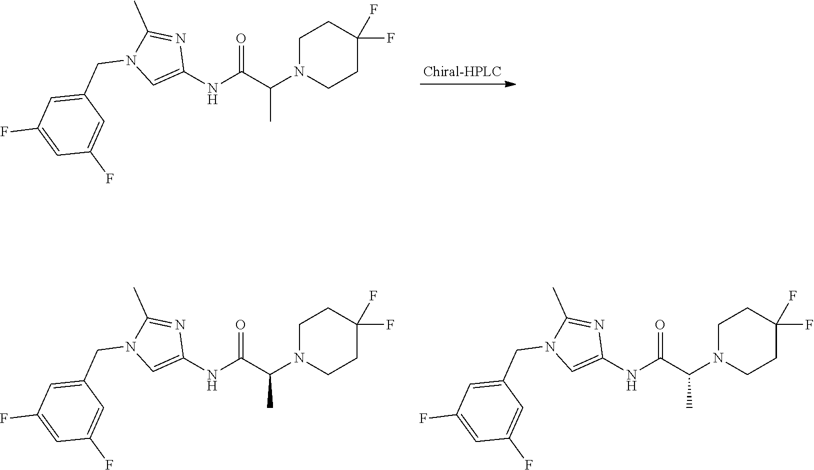 [SMILES: Cc1nc(NC(=O)C(C)N2CCC(F)(F)CC2)cn1Cc1cc(F)cc(F)c1 ; Cc1nc(NC(=O)[C@H](C)N2CCC(F)(F)CC2)cn1Cc1cc(F)cc(F)c1 ; Cc1nc(NC(=O)[C@@H](C)N2CCC(F)(F)CC2)cn1Cc1cc(F)cc(F)c1]

N-(1-(3,5-difluorobenzyl)-2-methyl-1H-imidazol-4-yl)-2-(4,4-difluoropiperidin-1-yl)propanamide (29.0 mg) was separated by prep chiral HPLC (Column: CHIRAL ART Cellulose-SZ, 3×25 cm, 5 μm; Mobile Phase A: Hex (10 mM NH₃-MeOH), Mobile Phase B: EtOH; Flow rate: 40 mL/min; Gradient: isocratic 30% B; Wave Length: 214/244 nm; RT1 (min): 6.2; RT2 (min): 8.4; Sample Solvent: ETOH:DCM=1:1; Injection Volume: 1 mL; Number Of Runs: 1) to afford (S)—N-(1-(3,5-difluorobenzyl)-2-methyl-1H-imidazol-4-yl)-2-(4,4-difluoropiperidin-1-yl)propanamide (Compound 21) (Method I, 0.74 min, peak 1, 14.1 mg, 100% e.e.) as a white solid and (R)—N-(1-(3,5-difluorobenzyl)-2-methyl-1H-imidazol-4-yl)-2-(4,4-difluoropiperidin-1-yl)propanamide (Compound 22) (Method I, 1.18 min, peak 2, 13.5 mg, 99.82% e.e.) as a white solid.

TABLE 10

| Compound# | Characterization Data |
|---|---|
| 21 | LCMS (ESI) [M + H]⁺: 399.1. |
| | ¹H NMR (400 MHz, Methanol-d₄) δ 7.24 (s, 1H), 6.89 (t, J = 9.2 Hz, 1H), 6.76 (d, J = 7.1 Hz, |
| | 2H), 5.17 (s, 2H), 3.40 (q, J = 7.0 Hz, 1H), 2.76-2.66 (m, 4H), 2.29 (s, 3H), 2.29-2.03 (m, |
| | 4H), 1.29 (d, J = 6.9 Hz, 3H). |
| 22 | LCMS (ESI) [M + H]⁺: 399.1. |
| | ¹H NMR (400 MHz, Methanol-d₄) δ 7.24 (s, 1H), 6.89 (td, J = 9.1, 4.5 Hz, 1H), 6.81-6.64 (m, |
| | 2H), 5.17 (s, 2H), 3.39 (q, J = 6.9 Hz, 1H), 2.76-2.65 (m, 4H), 2.08 (tt, J = 12.8, 5.7 Hz, 5H), |
| | 1.28 (d, J = 7.0 Hz, 4H). |

### Example 10: N-(1-(3,5-difluorobenzyl)-2-methyl-1H-imidazol-4-yl)-2-morpholinopropanamide

### Step 1: N-(1-(3,5-difluorobenzyl)-2-methyl-1H-imidazol-4-yl)-2-morpholinopropanamide

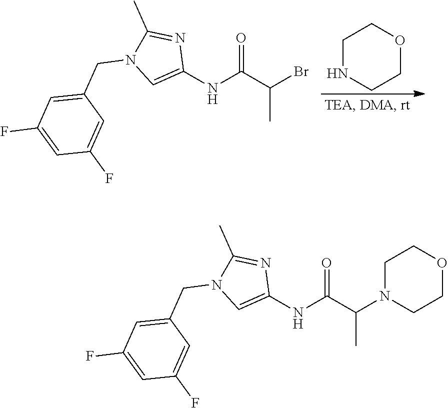

A mixture of 2-bromo-N-(1-(3,5-difluorobenzyl)-2-methyl-1H-imidazol-4-yl)propanamide (100 mg, 0.29 mmol), morpholine (48 mg, 0.58 mmol) and TEA (74 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford N-(1-(3,5-difluorobenzyl)-2-methyl-1H-imidazol-4-yl)-2-morpholinopropanamide (43.0 mg, 42% yield) as a white solid.

LCMS (ESI) [M+H]⁺: 365.17.

### Step 2: (S)—N-(1-(3,5-difluorobenzyl)-2-methyl-1H-imidazol-4-yl)-2-morpholinopropanamide and (R)—N-(1-(3,5-difluorobenzyl)-2-methyl-1H-imidazol-4-yl)-2-morpholinopropanamide

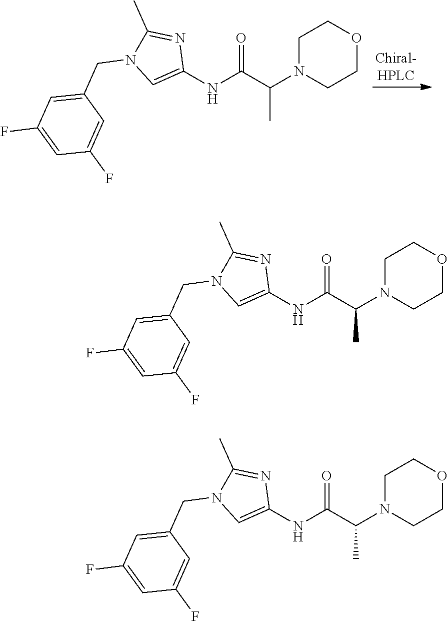

N-(1-(3,5-difluorobenzyl)-2-methyl-1H-imidazol-4-yl)-2-morpholinopropanamide (43.0 mg) was separated by prep chiral HPLC (Column: CHIRAL ART Cellulose-SZ, 3×25 cm, 5 μm; Mobile Phase A: Hex (10 mM NH₃-MeOH), Mobile Phase B: EtOH; Flow rate: 40 mL/min; Gradient: isocratic 30% B; Wave Length: 214/224 nm; RT1 (min): 8.6; RT2 (min): 11.8; Sample Solvent: EtOH DCM=3:2; Injection Volume: 1.2 mL; Number Of Runs: 2) to afford (S)—N-(1-(3,5-difluorobenzyl)-2-methyl-1H-imidazol-4-yl)-2-morpholinopropanamide (Compound 23) (Method I, 1.01 min, peak 1, 13.8 mg, 100% e.e.) as a white solid and (R)—N-(1-(3,5-difluorobenzyl)-2-methyl-1H-imidazol-4-yl)-2-morpholinopropanamide (Compound 24) (Method I, 1.39 min, peak 2, 10.3 mg, 99.9% e.e.) as a white solid.

TABLE 11

| Compound# | Characterization Data |
|---|---|
| 23 | LCMS (ESI) [M + H]⁺: 365.1. |
| | ¹H NMR (400 MHz, Methanol-d₄) δ 7.23 (s, 1H), 6.92-6.82 (m, 1H), 6.76 (d, J = 7.1 Hz, 2H), |
| | 5.17 (s, 2H), 3.89-3.70 (m, 4H), 3.21 (q, J = 7.0 Hz, 1H), 2.73-2.40 (m, 4H), 2.28 (s, 3H), |
| | 1.29 (d, J = 6.9 Hz, 3H). |
| 24 | LCMS (ESI) [M + H]⁺: 365.1. |
| | ¹H NMR (400 MHz, Methanol-d₄) δ 7.23 (s, 1H), 6.89 (tt, J = 9.0, 2.2 Hz, 1H), 6.74 (d, J = 2.2 |
| | Hz, 2H), 5.16 (s, 2H), 3.79-3.70 (m, 4H), 3.20 (q, J = 7.0 Hz, 1H), 2.79-2.42 (m, 4H), 2.28 (s, |
| | 3H), 1.28 (d, J = 6.9 Hz, 3H). |

### Example 11: 4-((3S)-1-(1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide

### Step 1: 4-nitro-2-(trifluoromethyl)-1H-imidazole

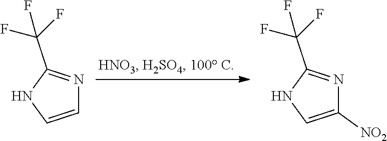 [SMILES: FC(F)(F)c1ncc[nH]1 ; O=[N+]([O-])c1c[nH]c(C(F)(F)F)n1]

2-(trifluoromethyl)-1H-imidazole (1.0 g, 7.35 mmol) was slowly added into a 20 mL sample vial containing HNO₃ (3 mL, 98% in H₂O) and H₂SO₄ (3 mL, 98% in H₂O). The resulting mixture was heated and stirred for 1 hour at 100° C. Upon completion of the reaction as monitored by TLC. The mixture was cooled to room temperature and slowly poured into ice water (40 mL). The white precipitate was collected by filtration and air dried to give crude 4-nitro-2-(trifluoromethyl)-1H-imidazole (1.5 g), which was further purified by flash column chromatography (PE/EA; 1:1) to afford 4-nitro-2-(trifluoromethyl)-1H-imidazole (800 mg, 61.5% yield) as a white solid. LCMS (ESI) [M+H]⁺: 182.

### Step 2: 1-[(3,5-difluorophenyl)methyl]-4-nitro-2-(trifluoromethyl)imidazole

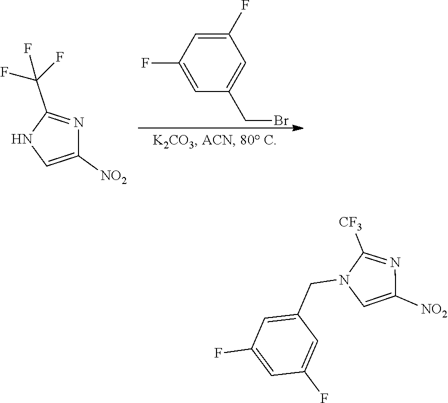 [SMILES: O=[N+]([O-])c1c[nH]c(C(F)(F)F)n1 ; Fc1cc(F)cc(CBr)c1 ; O=[N+]([O-])c1cn(Cc2cc(F)cc(F)c2)c(C(F)(F)F)n1]

A mixture of 4-nitro-2-(trifluoromethyl)-1H-imidazole (700 mg, 3.86 mmol, 1 equiv), 1-(bromomethyl)-3,5-difluorobenzene (800 mg, 3.866 mmol, 1 equiv), K₂CO₃ (1.60 g, 11.59 mmol, 3 equiv) in ACN (10 mL) was stirred for 2 h at 80° C. The resulting mixture was concentrated under reduced pressure. The residue was purified by silica gel column chromatography, eluted with CH₂Cl₂/PE (9:1) to afford 1-[(3,5-difluorophenyl)methyl]-4-nitro-2-(trifluoromethyl)imidazole (630 mg, 53% yield) as a white solid.

LCMS (ESI) [M+H]⁺: 308.

### Step 3: 1-(3,5-difluorobenzyl)-2-(trifluoromethyl)-1H-imidazol-4-amine

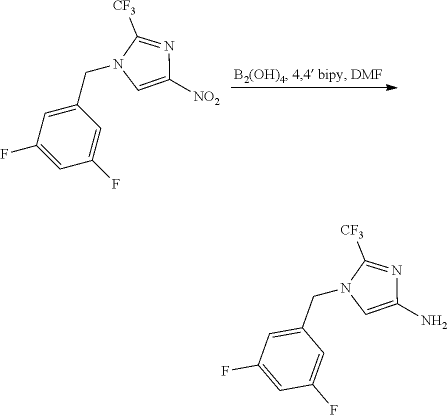 [SMILES: O=[N+]([O-])c1cn(Cc2cc(F)cc(F)c2)c(C(F)(F)F)n1 ; Nc1cn(Cc2cc(F)cc(F)c2)c(C(F)(F)F)n1]

A solution of 1-[(3,5-difluorophenyl)methyl]-4-nitro-2-(trifluoromethyl)imidazole (200 mg, 0.65 mmol, 1 equiv) and tetrahydroxydiboron (233 mg, 2.60 mmol, 4 equiv) in DMF (3 mL) was added 4,4′-Dipyridyl (30 mg, 0.19 mmol, 0.3 equiv) dropwise at 0° C. The resulting mixture was diluted with water (10 mL). The aqueous layer was extracted with EtOAc (3×20 mL). The resulting mixture was concentrated under reduced pressure. This resulted in 1-(3,5-difluorobenzyl)-2-(trifluoromethyl)-1H-imidazol-4-amine (180 mg, 99% yield) as a white solid. This crude product was carried to the next step without further purification.

LCMS (ESI) [M+H]⁺: 278.

### Step 4: 2-bromo-N-(1-(3,5-difluorobenzyl)-2-(trifluoromethyl)-1H-imidazol-4-yl)propanamide

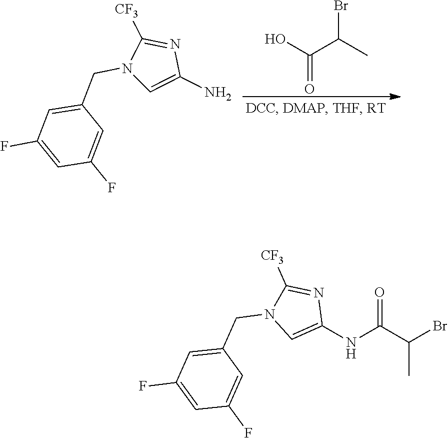

A solution of 1-(3,5-difluorobenzyl)-2-(trifluoromethyl)-1H-imidazol-4-amine (180 mg, 0.64 mmol, 1 equiv), DCC (160 mg, 0.77 mmol, 1.2 equiv), DMAP (39 mg, 0.32 mmol, 0.5 equiv) and α-bromopropionic acid (298 mg, 1.94 mmol, 3 equiv) in THF (5 mL) was stirred for 30 min at room temperature. The resulting mixture was concentrated under reduced pressure. The residue was purified by silica gel column chromatography, eluted with PE/EA (5:1) to afford 2-bromo-N-(1-(3,5-difluorobenzyl)-2-(trifluoromethyl)-1H-imidazol-4-yl)propanamide (201 mg, 75% yield) as a white solid.

LCMS (ESI) [M+H]⁺: 412

### Step 5: 4-((3S)-1-(1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide

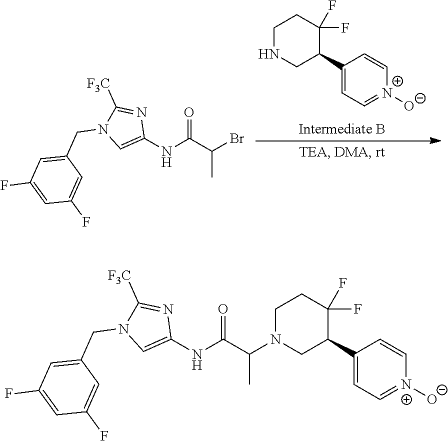 [SMILES: CC(Br)C(=O)Nc1cn(Cc2cc(F)cc(F)c2)c(C(F)(F)F)n1 ; [O-][n+]1ccc([C@H]2CNCCC2(F)F)cc1 ; CC(C(=O)Nc1cn(Cc2cc(F)cc(F)c2)c(C(F)(F)F)n1)N1CCC(F)(F)[C@@H](c2cc[n+]([O-])cc2)C1]

A mixture of 2-bromo-N-(1-(3,5-difluorobenzyl)-2-(trifluoromethyl)-1H-imidazol-4-yl)propanamide (100 mg, 0.29 mmol), (S)-4-(4,4-difluoropiperidin-3-yl)pyridine 1-oxide (Intermediate B) (52 mg, 0.24 mmol) and TEA (74 mg, 0.73 mmol) in DMA (3 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide (14 mg, 21% yield) as a white solid. LCMS (ESI) [M+H]⁺: 546.

### Step 6: 4-((S)-1-((S)-1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide and 4-((S)-1-((R)-1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide

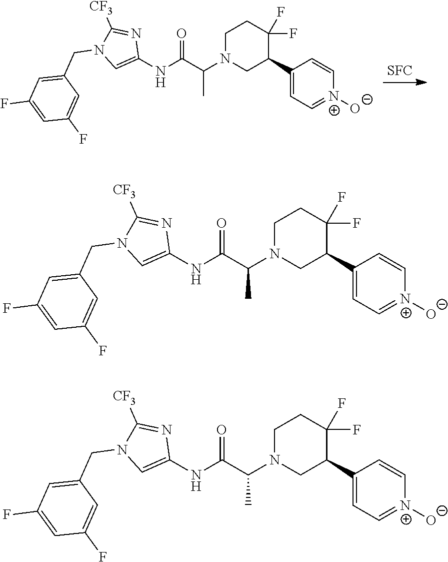

4-((3S)-1-(1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide (14 mg) was separated by Prep-Chiral HPLC with the following conditions Column: CHIRALPAK IE, 3×25 cm, 5 μm; Mobile Phase A: HEX:MtBE=1:1 (1:1 (0.5% 2M NH₃-MEOH), Mobile Phase B: MeOH; Flow rate: 40 mL/min; Gradient: isocratic 15% B; Wave Length: 217/270 nm; RT1 (min): 17.2; RT2 (min): 23.6; Sample Solvent: MeOH:DCM=8:1; Injection Volume: 1 mL; Number Of Runs: 2 to afford 4-((S)-1-((S)-1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide (Compound 25) (Method A, 0.93 min, peak 1, 2.0 mg, 100% e.e.) as a white solid and 4-((S)-1-((R)-1-((1-(3,5-difluorobenzyl)-2-(trifluoromethyl)-1H-imidazol-4-yl)amino)-1-oxopropan-2-yl)-4,4-difluoropiperidin-3-yl)pyridine 1-oxide (Compound 26) (Method A, 1.46 min, peak 2, 4.9 mg, 100% e.e.) as a white solid.

TABLE 12

| Compound# | Characterization Data |
|---|---|
| 25 | LCMS (ESI) [M + H]⁺: 546.3. |
| | 1H NMR (400 MHz, Methanol-d₄) δ 8.29 (d, J = 6.8 Hz, 2H), 7.59 − 7.54 (m, 3H), 6.94 (tt, J = |
| | 9.2, 2.3 Hz, 1H), 6.83 − 6.78 (m, 2H), 5.38 (s, 2H), 3.63 − 3.42 (m, 2H), 3.04 − 2.96 (m, 2H), |
| | 2.88 − 2.76 (m, 2H), 2.40 − 2.12 (m, 2H), 1.33 (d, J = 7.0 Hz, 3H). |
| 26 | LCMS (ESI) [M + H]⁺: 546.3. |
| | 1H NMR (400 MHz, Methanol- d₄) δ 8.29 (d, J = 6.8 Hz, 2H), 7.59 − 7.55 (m, 3H), 6.94 (tt, J = |
| | 9.1, 2.4 Hz, 1H), 6.83 − 6.77 (m, 2H), 5.39 (s, 2H), 3.64 − 3.52 (m, 2H), 3.04 − 2.93 (m, 2H), |
| | 2.64 (td, J = 11.6, 3.3 Hz, 1H), 2.30 − 2.16 (m, 2H), 1.33 (d, J = 7.0 Hz, 3H). |

### Example 12: 4-((3S)-1-(1-((2-(3,5-difluorobenzyl)oxazol-4-yl)amino)-1-oxopropan-2-yl)-4,4-difluoropiperidin-3-yl)pyridine 1-oxide

### Step 1: ethyl 2-(3,5-difluorobenzyl)oxazole-4-carboxylate

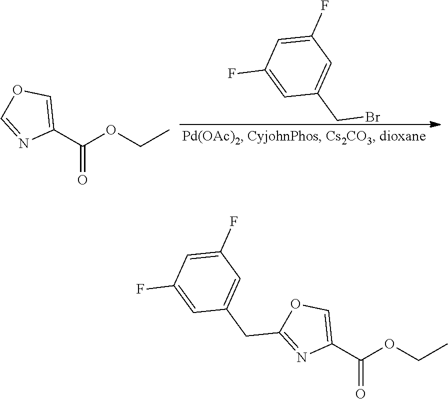

A solution of ethyl 1,3-oxazole-4-carboxylate (5 g, 35.42 mmol, 1 equiv) in dioxane (20 mL) was treated with 1-(bromomethyl)-3,5-difluorobenzene (8.8 g, 42.51 mmol, 1.2 equiv), Pd(OAc)₂ (0.8 g, 3.54 mmol, 0.1 equiv), Cs₂CO₃ (23.09 g, 70.85 mmol, 2 equiv), (2-Biphenyl)dicyclohexylphosphine (CyJohnPhos) (2.48 g, 7.08 mmol, 0.2 equiv) for 2 h at 100° C. The resulting mixture was concentrated under reduced pressure. The residue was purified by silica gel column chromatography, eluted with CH₂Cl₂/PE (5:1) to afford ethyl 2-(3,5-difluorobenzyl)oxazole-4-carboxylate (4.5 g, 47% yield) as a brown solid.

LCMS (ESI) [M+H]⁺: 268.

### Step 2: 2-(3,5-difluorobenzyl)oxazole-4-carboxylic acid

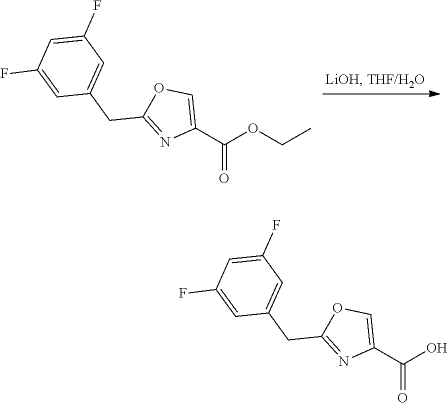 [SMILES: CCOC(=O)c1coc(Cc2cc(F)cc(F)c2)n1 ; O=C(O)c1coc(Cc2cc(F)cc(F)c2)n1]

A solution of ethyl 2-(3,5-difluorobenzyl)oxazole-4-carboxylate (4 g, 14.96 mmol, 1 equiv) and LiOH (1.79 g, 74.84 mmol, 5 equiv) in THF (20 mL) and H₂O (20 mL) was stirred for 1 h at room temperature. The mixture was acidified to pH 1 with conc. HCl. The resulting mixture was concentrated under reduced pressure to remove organic solvent. The precipitated solids were collected by filtration and washed with acetone. This resulted in 2-(3,5-difluorobenzyl)oxazole-4-carboxylic acid (3 g, 83% yield) as a brown solid.

LCMS (ESI) [M+H]⁺: 240.

### Step 3: tert-butyl (2-(3,5-difluorobenzyl)oxazol-4-yl)carbamate

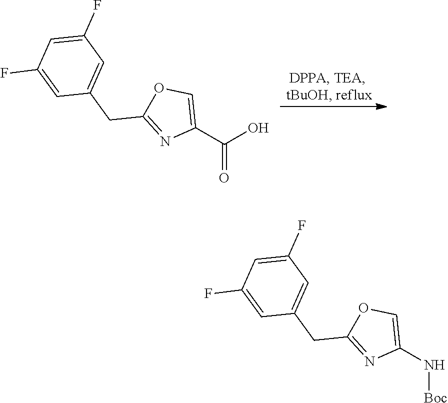 [SMILES: O=C(O)c1coc(Cc2cc(F)cc(F)c2)n1 ; CC(C)(C)OC(=O)Nc1coc(Cc2cc(F)cc(F)c2)n1]

A solution of 2-(3,5-difluorobenzyl)oxazole-4-carboxylic acid (3 g, 12.54 mmol, 1 equiv) in t-BuOH (50 mL) was treated with TEA (5.08 g, 50.17 mmol, 4 equiv) for 2 h at room temperature under nitrogen atmosphere followed by the addition of DPPA (10.36 g, 37.62 mmol, 3 equiv) dropwise at 0° C. The resulting mixture was stirred overnight at 100° C. The resulting mixture was concentrated under reduced pressure. The residue was purified by silica gel column chromatography, eluted with CH₂Cl₂/PE (3:1) to afford tert-butyl (2-(3,5-difluorobenzyl)oxazol-4-yl)carbamate (600 mg, 15% yield) as a brown solid.

LCMS (ESI) [M+H]⁺: 311.

### Step 4: tert-butyl (2-bromopropanoyl)(2-(3,5-difluorobenzyl)oxazol-4-yl)carbamate

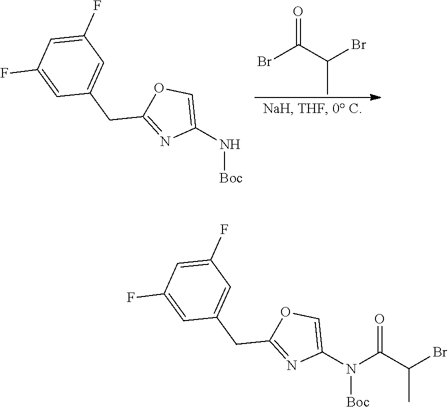

A solution of tert-butyl (2-(3,5-difluorobenzyl)oxazol-4-yl)carbamate (200 mg, 0.64 mmol, 1 equiv) in THF (3 mL) was treated with NaH (60% in mineral oil, 77 mg, 3.22 mmol, 5 equiv) for 15 min at 0° C. under nitrogen atmosphere followed by the addition of 2-bromopropanoyl bromide (417 mg, 1.93 mmol, 3 equiv) dropwise at 0° C. The reaction was quenched by the addition of MeOH. The resulting mixture was concentrated under reduced pressure. The residue was purified by silica gel column chromatography, eluted with CH₂Cl₂/PE (2:3) to afford tert-butyl (2-bromopropanoyl)(2-(3,5-difluorobenzyl)oxazol-4-yl)carbamate (170 mg, 59% yield) as a yellow solid.

LCMS (ESI) [M+H]⁺: 445.

### Step 5: 4-((3S)-1-(1-((tert-butoxycarbonyl)(2-(3,5-difluorobenzyl)oxazol-4-yl)amino)-1-oxopropan-2-yl)-4,4-difluoropiperidin-3-yl)pyridine 1-oxide

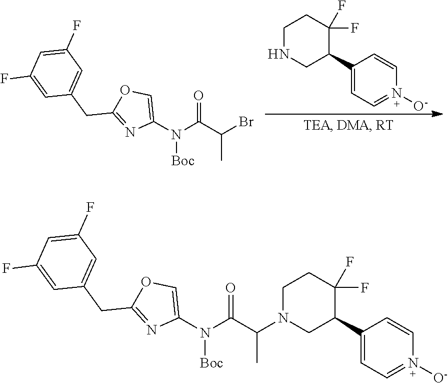

tert-butyl (2-bromopropanoyl)(2-(3,5-difluorobenzyl)oxazol-4-yl)carbamate (170 mg, 0.38 mmol, 1 equiv) in DMA (3 mL) was treated with (S)-4-(4,4-difluoropiperidin-3-yl)pyridine 1-oxide (Intermediate B) (81 mg, 0.38) and TEA (4 mg, 0.03 mmol, 0.1 equiv) and the mixture was stirred for 2 days at room temperature. The resulting solution was poured into water (10 mL) and extracted with CH₂Cl₂. The organic layer was concentrated under reduced pressure. The residue was purified by silica gel column chromatography, eluted with CH₂Cl₂/MeOH (15:1) to afford 4-((3S)-1-(1-((tert-butoxycarbonyl)(2-(3,5-difluorobenzyl)oxazol-4-yl)amino)-1-oxopropan-2-yl)-4,4-difluoropiperidin-3-yl)pyridine 1-oxide (90 mg, 40% yield) as a dark yellow oil.

LCMS (ESI) [M+H]⁺: 579.

### Step 6: 4-((3S)-1-(1-((2-(3,5-difluorobenzyl)oxazol-4-yl)amino)-1-oxopropan-2-yl)-4,4-difluoropiperidin-3-yl)pyridine 1-oxide

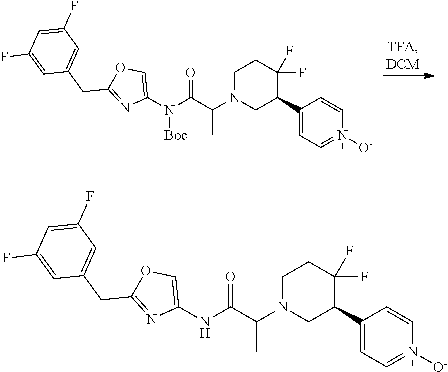

A solution of 4-((3S)-1-(1-((tert-butoxycarbonyl)(2-(3,5-difluorobenzyl)oxazol-4-yl)amino)-1-oxopropan-2-yl)-4,4-difluoropiperidin-3-yl)pyridine 1-oxide (90 mg, 0.15 mmol, 1 equiv) and TFA (2 mL) in DCM (2 mL) was stirred for 2 h at room temperature. The resulting mixture was concentrated under reduced pressure. The crude product (70 mg) was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 24% B to 44% B in 9 min; Wave Length: 254 nm/220 nm; RT1 (min): 8.25/8.55) to afford 4-((3S)-1-(1-((2-(3,5-difluorobenzyl)oxazol-4-yl)amino)-1-oxopropan-2-yl)-4,4-difluoropiperidin-3-yl)pyridine 1-oxide (35 mg, 47% yield) as a white solid.

LCMS (ESI) [M+H]⁺: 479.

### Step 7: 4-((S)-1-((S)-1-((2-(3,5-difluorobenzyl)oxazol-4-yl)amino)-1-oxopropan-2-yl)-4,4-difluoropiperidin-3-yl)pyridine 1-oxide and 4-((S)-1-((R)-1-((2-(3,5-difluorobenzyl)oxazol-4-yl)amino)-1-oxopropan-2-yl)-4,4-difluoropiperidin-3-yl)pyridine 1-oxide

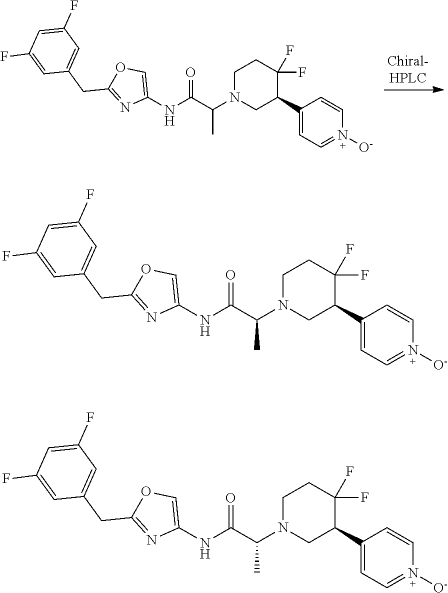

4-((3S)-1-(1-((2-(3,5-difluorobenzyl)oxazol-4-yl)amino)-1-oxopropan-2-yl)-4,4-difluoropiperidin-3-yl)pyridine 1-oxide (35 mg, 0.07 mmol, 1 equiv) was purified by Chiral-HPLC with the following conditions (Column: CHIRAL ART Cellulose-SB, 3×25 cm, 5 μm; Mobile Phase A: MtBE (10 mM NH₃-MeOH), Mobile Phase B: MEOH; Flow rate: 40 mL/min; Gradient: isocratic 10% B; Wave Length: 276/218 nm; RT1 (min): 14.16; RT2 (min): 17.24; Sample Solvent: MEOH; Injection Volume: 1.5 mL; Number Of Runs: 2) to afford 4-((S)-1-((S)-1-((2-(3,5-difluorobenzyl)oxazol-4-yl)amino)-1-oxopropan-2-yl)-4,4-difluoropiperidin-3-yl)pyridine 1-oxide (Compound 27) (Method J, 1.63 min, peak 1, 4.1 mg, 100% e.e.) as a white solid and 4-((S)-1-((R)-1-((2-(3,5-difluorobenzyl)oxazol-4-yl)amino)-1-oxopropan-2-yl)-4,4-difluoropiperidin-3-yl)pyridine 1-oxide (Compound 28) (Method J, 1.97 min, peak 2, 3.6 mg, 99.715% e.e.) as a white solid.

LCMS (ESI) [M+H]⁺: 479.

TABLE 13

| Compound# | Characterization Data |
|---|---|
| 27 | LCMS (ESI) [M + H]⁺: 479.2 |
| | ¹H NMR (400 MHz, Methanol-d₄) δ 8.28 (d, J = 7.2 Hz, 2H), 8.01 (d, J = 1.7 Hz, 1H), 7.56 (d, |
| | J = 6.5 Hz, 2H), 6.98 − 6.89 (m, 2H), 6.90 − 6.84 (m, 1H), 4.13 (s, 2H), 3.59 − 3.48 (m, 2H), |
| | 3.04 − 2.93 (m, 2H), 2.82 (t, J = 11.4 Hz, 2H), 2.40 − 2.11 (m, 2H), 1.32 (d, J = 6.7 Hz, 3H). |
| 28 | LCMS (ESI) [M + H]⁺: 479.2 |
| | ¹H NMR (400 MHz, Methanol-d₄) δ 8.29 (d, J = 6.8 Hz, 2H), 8.01 (s, 1H), 7.57 (d, J = 6.4 Hz, |
| | 2H), 6.94 (d, J = 7.3 Hz, 2H), 6.90 − 6.83 (m, 1H), 4.13 (s, 2H), 3.73 − 3.52 (m, 2H), 3.03 − |
| | 2.89 (m, 2H), 2.67 − 2.61 (m, 1H), 2.26 − 2.12 (m, 2H), 1.32 (d, J = 7.0 Hz, 3H). |

### Example 13: N-(1-(3,5-difluorobenzyl)-1H-imidazol-4-yl)-2,2-difluorospiro[2.3]hexane-1-carboxamide

### Step 1: ethyl 2,2-difluorospiro[2.3]hexane-1-carboxylate

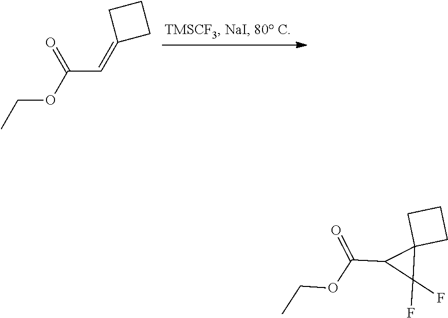

A mixture of ethyl 2-cyclobutylideneacetate (1 g, 7.13 mmol), sodium iodide (2.11 g, 14.26 mmol), trimethyl(trifluoromethyl)silane (3.03 g, 21.39 mmol) in freshly distilled THF (10 mL) was stirred for 14 h at 80° C. The resulting mixture was poured into water (40 mL) and extracted with PE (3×30 mL). The combined organic layers were dried with MgSO₄, filtered and concentrated under reduced pressure to afford crude ethyl 2,2-difluorospiro[2.3]hexane-1-carboxylate (1.2 g, 53% yield) as colorless oil, which was used in the next step without further purification. ¹H NMR (400 MHz, Chloroform-d) δ 4.17 (q, J=7.1 Hz, 2H), 3.17-3.08 (m, 2H), 2.95-2.76 (m, 2H), 2.63-2.19 (m, 1H), 2.11 (p, J=8.0 Hz, 2H), 1.27 (t, J=7.1 Hz, 3H).

### Step 2: 2,2-difluorospiro[2.3]hexane-1-carboxylic acid

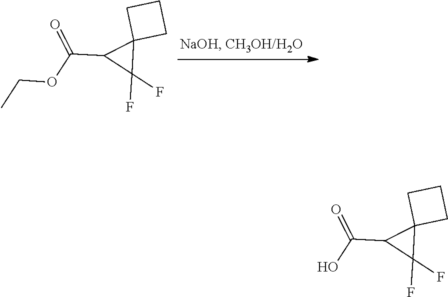

A mixture of ethyl 2,2-difluorospiro[2.3]hexane-1-carboxylate (1.2 g, 6.31 mmol), sodium hydroxide (2.52 g, 63.1 mmol) in methanol (10 mL) and water (10 mL) was stirred for 3 h at room temperature. The pH of the resulting mixture was adjusted to 3 and excess methanol was removed under reduced pressure. The crude residue was extracted with EA (3×10 mL). The combined organic layers were dried with MgSO₄, filtered and concentrated under reduced pressure to afford crude 2,2-difluorospiro[2.3]hexane-1-carboxylic acid (900 mg, 88% yield) as colorless oil, which was used in the next step without further purification.

LCMS (ESI) [M−H]⁺: 161.1.

### Step 3: 1-(3,5-difluorobenzyl)-4-nitro-1H-imidazole

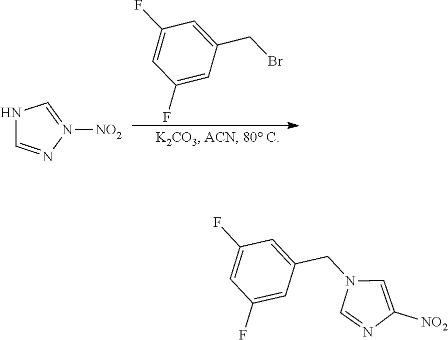

A mixture of 4-nitro-1H-imidazole (700 mg, 5.51 mmol), 1-(bromomethyl)-3,5-difluorobenzene (1.14 g, 5.51 mmol), potassium carbonate (1.60 g, 11.59 mmol) in ACN (10 mL) was stirred for 2 h at 80° C. The resulting mixture was concentrated under reduced pressure. The crude residue was purified by silica gel column chromatography, eluted with CH₂Cl₂/PE (9:1) to afford 1-(3,5-difluorobenzyl)-4-nitro-1H-imidazole (630 mg, 53% yield) as a white solid.

LCMS (ESI) [M+H]⁺: 240.1.

### Step 4: 1-(3,5-difluorobenzyl)-1H-imidazol-4-amine

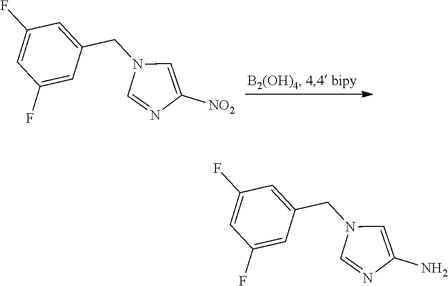

A solution of 1-(3,5-difluorobenzyl)-4-nitro-1H-imidazole (300 mg, 1.18 mmol) and tetrahydroxydiboron (466 mg, 4.80 mmol) in DMF (3 mL) was added 4-(pyridin-4-yl)pyridine (60 mg, 0.38 mmol) dropwise at 0° C. The resulting mixture was stirred at 0° C. for 15 minutes. Upon completion, the mixture washed with water. The aqueous layer was extracted with EtOAc. The combined organic layers were concentrated under reduced pressure to afford 1-(3,5-difluorobenzyl)-1H-imidazol-4-amine (180 mg, 68% yield) as a white solid, which was used in the next step without further purification.

LCMS (ESI) [M+H]⁺: 210.1.

### Step 5: N-(1-(3,5-difluorobenzyl)-1H-imidazol-4-yl)-2,2-difluorospiro[2.3]hexane-1-carboxamide

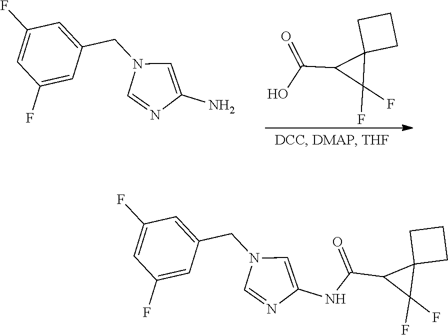 [SMILES: Nc1cn(Cc2cc(F)cc(F)c2)cn1 ; O=C(O)C1C(F)(F)C12CCC2 ; O=C(Nc1cn(Cc2cc(F)cc(F)c2)cn1)C1C(F)(F)C12CCC2]

A solution of 1-(3,5-difluorobenzyl)-1H-imidazol-4-amine (180 mg, 0.80 mmol), DCC (180 mg, 0.95 mmol), DMAP (50 mg, 0.5 mmol) and 2,2-difluorospiro[2.3]hexane-1-carboxylic acid (298 mg, 1.94 mmol) in THF (5 mL) was stirred for 30 min at room temperature. The resulting mixture was concentrated under reduced pressure. The crude residue was purified by silica gel column chromatography, eluted with PE/EA (5:1) to afford N-(1-(3,5-difluorobenzyl)-1H-imidazol-4-yl)-2,2-difluorospiro[2.3]hexane-1-carboxamide (14.1 mg, 4.64% yield) as a white solid.

LCMS (ESI) [M+H]⁺: 354.1

### Step 6: (R)—N-(1-(3,5-difluorobenzyl)-1H-imidazol-4-yl)-2,2-difluorospiro[2.3]hexane-1-carboxamide and (S)—N-(1-(3,5-difluorobenzyl)-1H-imidazol-4-yl)-2,2-difluorospiro[2.3]hexane-1-carboxamide

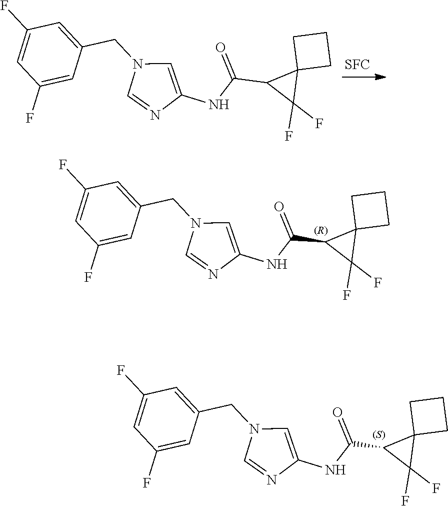

N-(1-(3,5-difluorobenzyl)-1H-imidazol-4-yl)-2,2-difluorospiro[2.3]hexane-1-carboxamide (8.0 mg) was separated by prep chiral HPLC (Column: CHIRAL ART Cellulose-SZ, 3×25 cm, 5 μm; Mobile Phase A: Hex (10 mM NH3-MeOH), Mobile Phase B: EtOH; Flow rate: 40 mL/min; Gradient: isocratic 20% B; Wave Length: 240/208 nm; RT1 (min): 5.54; RT2 (min): 7.54) to afford (R)—N-(1-(3,5-difluorobenzyl)-1H-imidazol-4-yl)-2,2-difluorospiro[2.3]hexane-1-carboxamide (Compound 29) (2.6 mg, 100 e.e.) as a white solid and (S)—N-(1-(3,5-difluorobenzyl)-1H-imidazol-4-yl)-2,2-difluorospiro[2.3]hexane-1-carboxamide (Compound 30) (2.6 mg, 99.9% e.e.) as a white solid.

TABLE 14

| Compound# | Characterization Data |
|---|---|
| 29 | LCMS (ESI) [M + H]⁺: 354.12. |
| | ¹H NMR (400 MHz, Methanol-d₄) δ 7.58 (d, J = 1.6 Hz, 1H), 7.23 (d, J = 1.6 Hz, 1H), 7.03 − |
| | 6.68 (m, 3H), 5.19 (s, 2H), 2.60 − 2.42 (m, 1H), 2.42 − 2.21 (m, 4H), 2.09 (dddq, J = 15.5, 9.3, |
| | 6.1, 3.1 Hz, 2H). |
| 30 | LCMS (ESI) [M + H]⁺: 354.12. |
| | ¹H NMR (400 MHz, Methanol-d₄) δ 7.58 (d, J = 1.6 Hz, 1H), 7.23 (d, J = 1.6 Hz, 1H), 7.04 − |
| | 6.70 (m, 3H), 5.19 (s, 2H), 2.61 − 2.44 (m, 1H), 2.43 − 2.20 (m, 4H), 2.08 (dtt, J = 7.7, 5.6, 4.4 |
| | Hz, 2H). |

### Example 14: 4-((S)-1-((S)-1-((1-(4-chloro-2-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(4-chloro-2-fluorobenzyl)-1H-imidazol-4-yl)amino)-1-oxopropan-2-yl)-4,4-difluoropiperidin-3-yl)pyridine 1-oxide

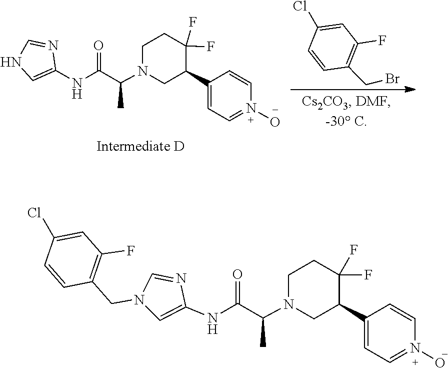

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.12 mmol, 1.5 equiv) in DMF (0.5 mL) were added 1-(bromomethyl)-4-chloro-2-fluorobenzene (25 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-1-((S)-1-((1-(4-chloro-2-fluorobenzyl)-1H-imidazol-4-yl)amino)-1-oxopropan-2-yl)-4,4-difluoropiperidin-3-yl)pyridine 1-oxide (Compound 31) (14.4 mg, 34% yield) as a white solid.

LCMS (ESI) [M+H]⁺: 494.1

¹H NMR (400 MHz, Methanol-d₄) δ 8.28 (d, J=6.8 Hz, 2H), 7.64-7.51 (m, 3H), 7.37-7.09 (m, 4H), 5.23 (s, 2H), 3.73-3.48 (m, 2H), 3.06-3.00 (m, 2H), 2.66-2.58 (m, 1H), 2.31-2.09 (m, 2H), 1.31 (d, J=6.9 Hz, 3H).

### Example 15: 4-[(3S)-4,4-difluoro-1-[(1S)-1-({1-[(2,3,4,5-tetrafluorophenyl)methyl]imidazol-4-yl}carbamoyl)ethyl]piperidin-3-yl]pyridin-1-ium-1-olate

### Step 1: 4-[(3S)-4,4-difluoro-1-[(1S)-1-({1-[(2,3,4,5-tetrafluorophenyl)methyl]imidazol-4-yl}carbamoyl)ethyl]piperidin-3-yl]pyridin-1-ium-1-olate

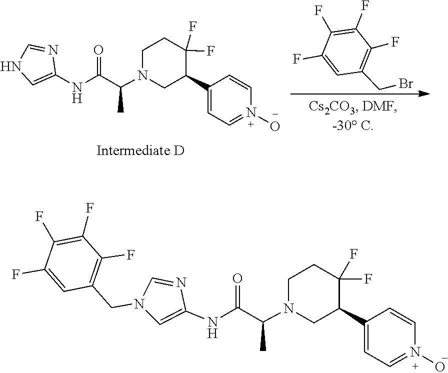

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.13 mmol, 1.5 equiv) in DMF (0.5 mL) were added 1-(bromomethyl)-2,3,4,5-tetrafluorobenzene (23 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-[(3S)-4,4-difluoro-1-[(1S)-1-({1-[(2,3,4,5-tetrafluorophenyl)methyl]imidazol-4-yl}carbamoyl)ethyl]piperidin-3-yl]pyridin-1-ium-1-olate (Compound 32) (19.4 mg, 44% yield) as a white solid.

LCMS [M+H]⁺: 514.2

¹H NMR (400 MHz, DMSO-d₆) δ 10.22 (s, 1H), 8.17 (d, J=6.8 Hz, 2H), 7.60 (d, J=1.5 Hz, 1H), 7.51-7.43 (m, 1H), 7.37 (d, J=6.6 Hz, 2H), 7.29 (d, J=1.5 Hz, 1H), 5.25 (s, 2H), 3.55 (d, J=6.9 Hz, 1H), 3.45 (d, J=26.0 Hz, 2H), 3.02-2.82 (m, 3H), 2.17-1.98 (m, 2H), 1.18 (s, 3H).

### Example 16: 4-((S)-1-((S)-1-((1-(5-chloro-2-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(5-chloro-2-fluorobenzyl)-1H-imidazol-4-yl)amino)-1-oxopropan-2-yl)-4,4-difluoropiperidin-3-yl)pyridine 1-oxide

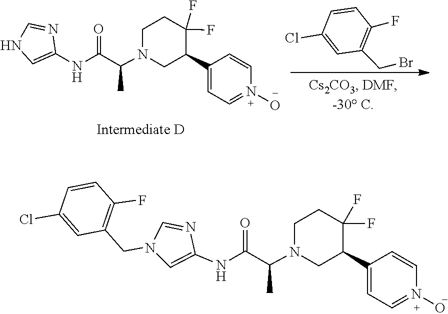

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.13 mmol, 1.5 equiv) in DMF (0.5 mL) were added 2-(bromomethyl)-4-chloro-1-fluorobenzene (25 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 2% B to 2% B in 1.5 min, 2% B to 15% B in 2 min, 15% B to 36% B in 9 min; Wave Length: 254 nm/220 nm; RT1 (min): 8.6) to afford 4-((S)-1-((S)-1-((1-(5-chloro-2-fluorobenzyl)-1H-imidazol-4-yl)amino)-1-oxopropan-2-yl)-4,4-difluoropiperidin-3-yl)pyridine 1-oxide (Compound 33) (6.8 mg, 15% yield) as a white solid.

LCMS [M+H]⁺: 494.2

¹H NMR (300 MHz, Methanol-d₄) δ 8.29 (d, J=6.6 Hz, 2H), 7.66-7.50 (m, 3H), 7.45-7.26 (m, 3H), 7.16 (t, J=9.1 Hz, 1H), 5.24 (s, 2H), 3.73-3.41 (m, 2H), 3.10-2.84 (m, 3H), 2.68-2.59 (m, 1H), 2.46-2.05 (m, 2H), 1.32 (d, J=7.0 Hz, 3H).

### Example 17: 4-((S)-4,4-difluoro-1-((S)-1-oxo-1-((1-(3,4,5-trifluorobenzyl)-1H-imidazol-4-yl)amino)propan-2-yl)piperidin-3-yl)pyridine 1-oxide

### Step 1: 4-((S)-4,4-difluoro-1-((S)-1-oxo-1-((1-(3,4,5-trifluorobenzyl)-1H-imidazol-4-yl)amino)propan-2-yl)piperidin-3-yl)pyridine 1-oxide

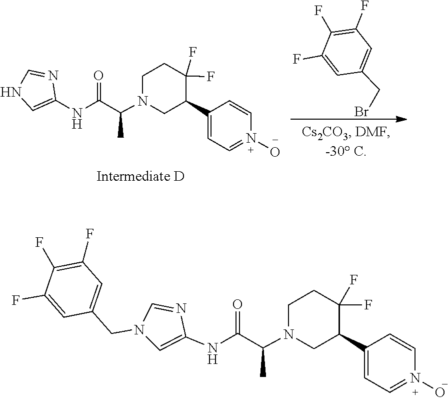

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.13 mmol, 1.5 equiv) in DMF (0.5 mL) were added 5-(bromomethyl)-1,2,3-trifluorobenzene (25 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: Kinetex EVO C18 Column, 30×150, Sum; Mobile Phase A: Water (0.1% FA), Mobile Phase B: ACN; Flow rate: 60 mL/min; Gradient: 5% B to 5% B in 1 min, 5% B to 14% B in 2 min, 14% to 33% B in 10 min; Wave Length: 254 nm/220 nm; RT1 (min): 9.33) to afford 4-((S)-4,4-difluoro-1-((S)-1-oxo-1-((1-(3,4,5-trifluorobenzyl)-1H-imidazol-4-yl)amino)propan-2-yl)piperidin-3-yl)pyridine 1-oxide (Compound 34) (13.2 mg, 31% yield) as a white solid.

LCMS [M+H]⁺: 496.2

¹H NMR (300 MHz, Methanol-d₄) δ 8.29 (d, J=7.2 Hz, 2H), 7.66-7.54 (m, 3H), 7.30 (d, J=1.6 Hz, 1H), 7.07 (t, J=6.9 Hz, 2H), 5.17 (s, 2H), 3.73-3.46 (m, 2H), 3.11-2.86 (m, 3H), 2.66-2.53 (m, 1H), 2.40-2.04 (m, 2H), 1.32 (d, J=7.0 Hz, 3H).

### Example 18: 4-((S)-1-((S)-1-((1-(2-chloro-5-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2-chloro-5-fluorobenzyl)-1H-imidazol-4-yl)amino)-1-oxopropan-2-yl)-4,4-difluoropiperidin-3-yl)pyridine 1-oxide

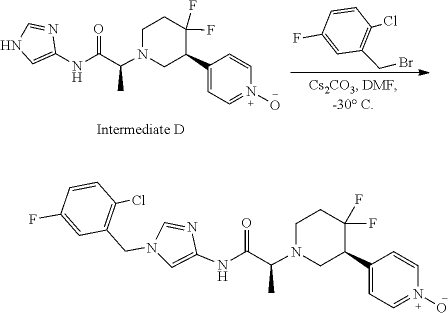

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.12 mmol, 1.5 equiv) in DMF (0.5 mL) was added 2-(bromomethyl)-1-chloro-4-fluorobenzene (22 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: Sunfire prep C18 column 30×150 mm, 5 μm; Mobile Phase A: Water (0.1% FA), Mobile Phase B: ACN; Flow rate: 60 mL/min; Gradient: 14% B to 34% B in 10 min; Wave Length: 254 nm/220 nm; RT1 (min): 10.7) to afford 4-((S)-1-((S)-1-((1-(2-chloro-5-fluorobenzyl)-1H-imidazol-4-yl)amino)-1-oxopropan-2-yl)-4,4-difluoropiperidin-3-yl)pyridine 1-oxide (Compound 35) (21.7 mg, 51% yield) as a white solid.

LCMS [M+H]⁺: 494.1

¹H NMR (400 MHz, Methanol-d₄) δ 8.29 (d, J=5.1 Hz, 2H), 7.62 (d, J=1.7 Hz, 1H), 7.57 (d, J=6.4 Hz, 2H), 7.47 (dd, J=8.9, 5.0 Hz, 1H), 7.30 (d, J=1.7 Hz, 1H), 7.16-7.10 (m, 1H), 6.97 (d, J=8.9 Hz, 1H), 5.31 (s, 2H), 3.64-3.49 (m, 2H), 3.08-2.91 (m, 1H), 2.74-2.62 (m, 1H), 2.40-2.12 (m, 2H), 1.32 (d, J=6.8 Hz, 3H).

### Example 19: 4-((S)-4,4-difluoro-1-((S)-1-((1-(4-fluorobenzyl)-1H-imidazol-4-yl)amino)-1-oxopropan-2-yl)piperidin-3-yl)pyridine 1-oxide

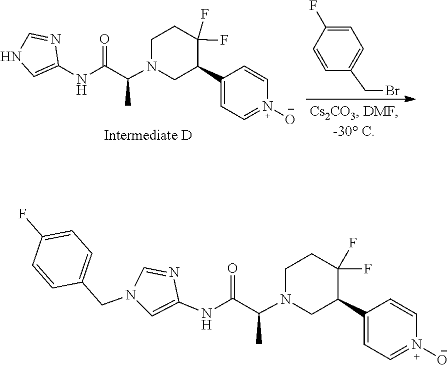 [SMILES: C[C@@H](C(=O)Nc1c[nH]cn1)N1CCC(F)(F)[C@@H](c2cc[n+]([O-])cc2)C1 ; Fc1ccc(CBr)cc1 ; C[C@@H](C(=O)Nc1cn(Cc2ccc(F)cc2)cn1)N1CCC(F)(F)[C@@H](c2cc[n+]([O-])cc2)C1]

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.13 mmol, 1.5 equiv) in DMF (0.5 mL) were added 1-(bromomethyl)-4-fluorobenzene (20 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.35) to afford 4-((S)-4,4-difluoro-1-((S)-1-((1-(4-fluorobenzyl)-1H-imidazol-4-yl)amino)-1-oxopropan-2-yl)piperidin-3-yl)pyridine 1-oxide (Compound 36) (11.4 mg, 28% yield) as a white solid.

LCMS [M+H]⁺: 460.2

¹H NMR (400 MHz, Methanol-d₄) δ 8.29 (d, J=5.1 Hz, 2H), 7.65-7.56 (m, 3H), 7.35-7.27 (m, 3H), 7.08 (t, J=6.6 Hz, 2H), 5.15 (s, 2H), 3.70-3.56 (m, 1H), 3.55-3.45 (m, 1H), 3.02 (s, 1H), 3.00 (d, J=3.5 Hz, 1H), 2.97-2.84 (m, 1H), 2.69-2.58 (m, 1H), 2.36-2.15 (m, 2H), 1.31 (d, J=7.0 Hz, 3H).

### Example 20: 4-((S)-4,4-difluoro-1-((S)-1-((1-(2-fluorobenzyl)-1H-imidazol-4-yl)amino)-1-oxopropan-2-yl)piperidin-3-yl)pyridine 1-oxide

### Step 1: 4-((S)-4,4-difluoro-1-((S)-1-((1-(2-fluorobenzyl)-1H-imidazol-4-yl)amino)-1-oxopropan-2-yl)piperidin-3-yl)pyridine 1-oxide

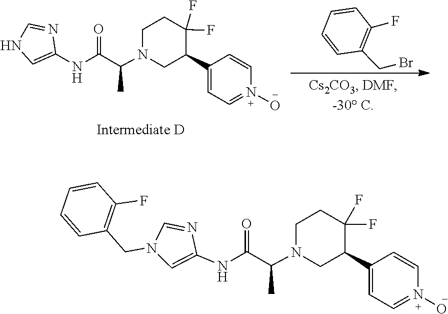

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.13 mmol, 1.5 equiv) in DMF (0.5 mL) was added 1-(bromomethyl)-2-fluorobenzene (18 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.65) to afford 4-((S)-4,4-difluoro-1-((S)-1-((1-(2-fluorobenzyl)-1H-imidazol-4-yl)amino)-1-oxopropan-2-yl)piperidin-3-yl)pyridine 1-oxide (Compound 37) (5.5 mg, 13% yield) as a white solid.

LCMS [M+H]⁺: 460.2

¹H NMR (400 MHz, Methanol-d₄) δ 8.29 (d, J=6.8 Hz, 2H), 7.71-7.54 (m, 3H), 7.48-7.29 (m, 3H), 7.26-7.03 (m, 2H), 5.25 (s, 2H), 3.72-3.43 (m, 2H), 3.06-3.01 (m, 2H), 2.96-3.89 (m, 1H), 2.70-2.58 (m, 1H), 2.38-2.13 (m, 2H), 1.31 (d, J=7.0 Hz, 3H).

### Example 21: 4-((S)-4,4-difluoro-1-((S)-1-((1-(3-fluorobenzyl)-1H-imidazol-4-yl)amino)-1-oxopropan-2-yl)piperidin-3-yl)pyridine 1-oxide

### Step 1: 4-((S)-4,4-difluoro-1-((S)-1-((1-(3-fluorobenzyl)-1H-imidazol-4-yl)amino)-1-oxopropan-2-yl)piperidin-3-yl)pyridine 1-oxide

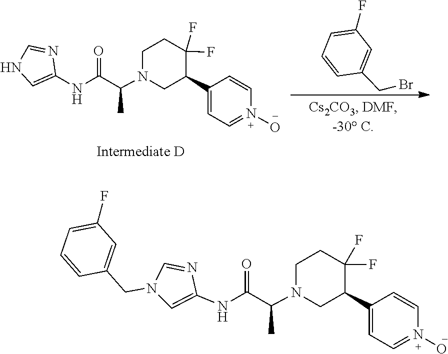

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (42 mg, 0.13 mmol, 1.5 equiv) in DMF (0.5 mL) was added 1-(bromomethyl)-3-fluorobenzene (21 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.65) to afford 4-((S)-4,4-difluoro-1-((S)-1-((1-(3-fluorobenzyl)-1H-imidazol-4-yl)amino)-1-oxopropan-2-yl)piperidin-3-yl)pyridine 1-oxide (Compound 38) (11.9 mg, 29% yield) as a white solid.

LCMS [M+H]⁺: 460.3

¹H NMR (400 MHz, Methanol-d₄) δ 8.29 (d, J=7.2 Hz, 2H), 7.66-7.50 (m, 3H), 7.45-7.24 (m, 2H), 7.18-6.88 (m, 3H), 5.20 (s, 2H), 3.71-3.44 (m, 2H), 3.09-2.87 (m, 3H), 2.71-2.54 (m, 1H), 2.41-2.07 (m, 2H), 1.32 (d, J=7.0 Hz, 3H).

### Example 22: 4-((S)-4,4-difluoro-1-((S)-1-oxo-1-((1-(2,3,5-trifluorobenzyl)-1H-imidazol-4-yl)amino)propan-2-yl)piperidin-3-yl)pyridine 1-oxide

### Step 1: 4-((S)-4,4-difluoro-1-((S)-1-oxo-1-((1-(2,3,5-trifluorobenzyl)-1H-imidazol-4-yl)amino)propan-2-yl)piperidin-3-yl)pyridine 1-oxide

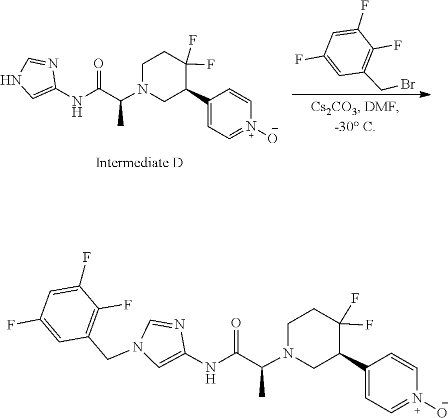

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.13 mmol, 1.5 equiv) in DMF (0.5 mL) were added 1-(bromomethyl)-2,3,5-trifluorobenzene (25 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.65) to afford 4-((S)-4,4-difluoro-1-((S)-1-oxo-1-((1-(2,3,5-trifluorobenzyl)-1H-imidazol-4-yl)amino)propan-2-yl)piperidin-3-yl)pyridine 1-oxide (Compound 39) (7.2 mg, 16% yield) as a white solid.

LCMS [M+H]⁺: 496.1

¹H NMR (400 MHz, Methanol-d₄) δ 8.29 (d, J=6.5 Hz, 2H), 7.70-7.53 (m, 3H), 7.33 (s, 1H), 7.21-7.16 (m, 1H), 6.93-6.82 (m, 1H), 5.30 (s, 2H), 3.64-3.49 (m, 2H), 3.08-3.00 (m, 2H), 2.99-2.93 (m, 1H), 2.66-2.61 (m, 1H), 2.42-2.09 (m, 2H), 1.33 (d, J=6.7 Hz, 3H).

### Example 23: 4-((S)-1-((S)-1-((1-(2,5-difluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2,5-difluorobenzyl)-1H-imidazol-4-yl)amino)-1-oxopropan-2-yl)-4,4-difluoropiperidin-3-yl)pyridine 1-oxide

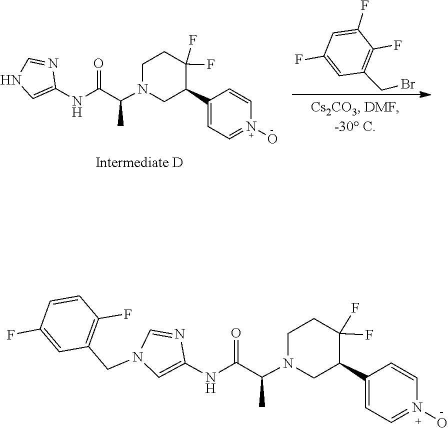

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.12 mmol, 1.5 equiv) in DMF (0.5 mL) were added 2-(bromomethyl)-1,4-difluorobenzene (21 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: Kinetex EVO C18 Column, 30×150, 5 um; Mobile Phase A: Water (0.1% FA), Mobile Phase B: ACN; Flow rate: 60 mL/min; Gradient: 5% B to 5% B in 1 min, 5% B to 10% B in 2 min, 10% to 15% B in 10 min; Wave Length: 254 nm/220 nm; RT1 (min): 8.78) to afford 4-((S)-1-((S)-1-((1-(2,5-difluorobenzyl)-1H-imidazol-4-yl)amino)-1-oxopropan-2-yl)-4,4-difluoropiperidin-3-yl)pyridine 1-oxide (Compound 40) (4.6 mg, 11% yield) as a white solid.

LCMS [M+H]⁺: 478.1

¹H NMR (300 MHz, Methanol-d₄) δ 8.29 (d, J=7.1 Hz, 2H), 7.63 (s, 1H), 7.60 (d, J=6.8 Hz, 2H), 7.33 (d, J=1.6 Hz, 1H), 7.24-7.03 (m, 2H), 5.26 (s, 2H), 3.66-3.52 (m, 1H), 3.50-3.42 (m, 1H), 3.08-2.92 (m, 1H), 2.74-2.54 (m, 1H), 2.34-2.12 (m, 2H), 1.34 (d, J=7.0 Hz, 3H).

### Example 24: 4-((S)-4,4-difluoro-1-((S)-1-oxo-1-((1-(2-(trifluoromethyl)benzyl)-1H-imidazol-4-yl)amino)propan-2-yl)piperidin-3-yl)pyridine 1-oxide

### Step 1: 4-((S)-4,4-difluoro-1-((S)-1-oxo-1-((1-(2-(trifluoromethyl)benzyl)-1H-imidazol-4-yl)amino)propan-2-yl)piperidin-3-yl)pyridine 1-oxide

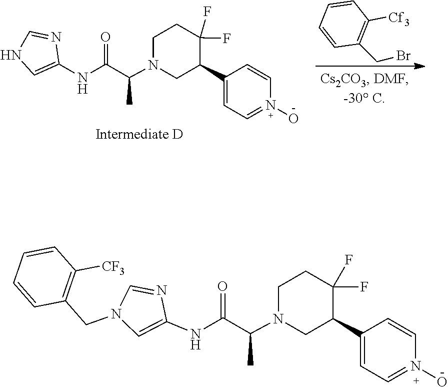

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.12 mmol, 1.5 equiv) in DMF (0.5 mL) was added 1-(bromomethyl)-2-(trifluoromethyl)benzene (25 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: Kinetex EVO C18 Column, 30×150, Sum; Mobile Phase A: Water (0.1% FA), Mobile Phase B: ACN; Flow rate: 60 mL/min; Gradient: 5% B to 5% B in 1 min, 5% B to 15% B in 2 min, 15% to 35% B in 10 min; Wave Length: 254 nm/220 nm; RT1 (min): 8.9) to afford 4-((S)-4,4-difluoro-1-((S)-1-oxo-1-((1-(2-(trifluoromethyl)benzyl)-1H-imidazol-4-yl)amino)propan-2-yl)piperidin-3-yl)pyridine 1-oxide (Compound 41) (11 mg, 25% yield) as a white solid.

LCMS [M+H]⁺: 510.2

¹H NMR (300 MHz, Methanol-d₄) δ 8.29 (d, J=6.9 Hz, 2H), 7.83-7.76 (m, 1H), 7.69-7.49 (m, 5H), 7.31-7.17 (m, 2H), 5.43 (s, 2H), 3.65-3.49 (m, 2H), 3.09-2.96 (m, 2H), 2.78-2.60 (m, 1H), 2.35-2.06 (m, 2H), 1.34 (d, J=7.0 Hz, 3H).

### Example 25: 4-((S)-4,4-difluoro-1-((S)-1-oxo-1-((1-(2,3,4-trifluorobenzyl)-1H-imidazol-4-yl)amino)propan-2-yl)piperidin-3-yl)pyridine 1-oxide

### Step 1: 4-((S)-4,4-difluoro-1-((S)-1-oxo-1-((1-(2,3,4-trifluorobenzyl)-1H-imidazol-4-yl)amino)propan-2-yl)piperidin-3-yl)pyridine 1-oxide

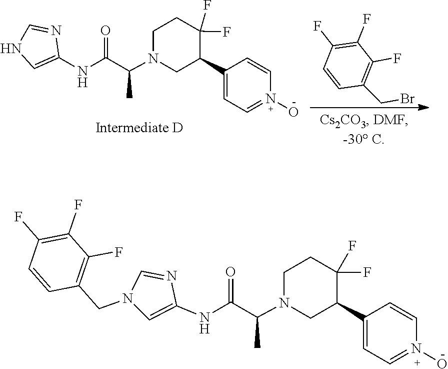

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (42 mg, 0.13 mmol, 1.5 equiv) in DMF (0.5 mL) was added 1-(bromomethyl)-2,3,4-trifluorobenzene (21 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.55) to afford 4-((S)-4,4-difluoro-1-((S)-1-oxo-1-((1-(2,3,4-trifluorobenzyl)-1H-imidazol-4-yl)amino)propan-2-yl)piperidin-3-yl)pyridine 1-oxide (Compound 42) (7.2 mg, 16% yield) as a white solid.

LCMS [M+H]⁺: 496.1

¹H NMR (400 MHz, Methanol-d₄) δ 8.29 (d, J=6.2 Hz, 2H), 7.72-7.51 (m, 3H), 7.29 (s, 1H), 7.19-7.07 (m, 2H), 5.27 (s, 2H), 3.70-3.43 (m, 2H), 3.09-2.90 (d, m, 2H), 2.69-2.54 (m, 1H), 2.42-2.09 (m, 2H), 1.31 (d, J=6.7 Hz, 3H).

### Example 26: 4-((S)-1-((S)-1-((1-(3-chlorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3-chlorobenzyl)-1H-imidazol-4-yl)amino)-1-oxopropan-2-yl)-4,4-difluoropiperidin-3-yl)pyridine 1-oxide

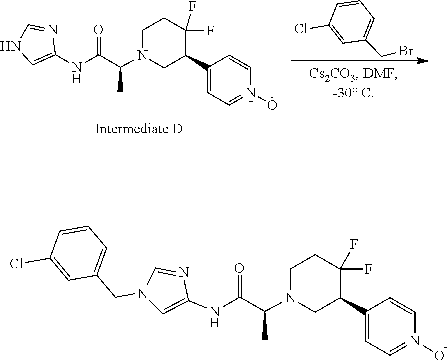

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.12 mmol, 1.5 equiv) in DMF (0.5 mL) were added 1-(bromomethyl)-3-chlorobenzene (21 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 1% B to 1% B in 1.5 min, 1% B to 15% B in 2 min, 15% B to 35% B in 9 min; Wave Length: 254 nm/220 nm; RT1 (min): 8.88): 9) to afford 4-((S)-1-((S)-1-((1-(3-chlorobenzyl)-1H-imidazol-4-yl)amino)-1-oxopropan-2-yl)-4,4-difluoropiperidin-3-yl)pyridine 1-oxide (Compound 43) (16.9 mg, 40% yield) as a white solid.

LCMS [M+H]⁺: 476.0

¹H NMR (300 MHz, Methanol-d₄) δ 8.31 (d, J=6.2 Hz, 2H), 7.66 (s, 1H), 7.62-7.57 (m, 2H), 7.40-7.35 (m, 2H), 7.31 (s, 2H), 7.28-7.21 (m, 1H), 5.21 (s, 2H), 3.73-3.47 (m, 2H), 3.09-2.90 (m, 1H), 2.75-2.60 (m, 1H), 2.42-2.11 (m, 2H), 1.34 (d, J=6.9 Hz, 3H).

### Example 27: 4-((S)-1-((S)-1-((1-(4-chloro-3,5-difluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(4-chloro-3,5-difluorobenzyl)-1H-imidazol-4-yl)amino)-1-oxopropan-2-yl)-4,4-difluoropiperidin-3-yl)pyridine 1-oxide

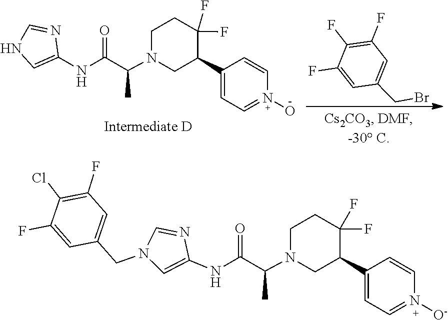

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.12 mmol, 1.5 equiv) in DMF (0.5 mL) was added 5-(bromomethyl)-2-chloro-1,3-difluorobenzene (25 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 17% B to 36% B in 8 min; RT1 (min): 9.52) to afford 4-((S)-1-((S)-1-((1-(4-chloro-3,5-difluorobenzyl)-1H-imidazol-4-yl)amino)-1-oxopropan-2-yl)-4,4-difluoropiperidin-3-yl)pyridine 1-oxide (Compound 44) (15.2 mg, 34% yield) as a light yellow solid.

LCMS [M+H]⁺: 512.2

¹H NMR (400 MHz, DMSO-d₆) δ 10.20 (s, 1H), 8.17 (d, J=7.2 Hz, 2H), 7.64 (d, J=1.6 Hz, 1H), 7.43-7.18 (m, 5H), 5.17 (s, 2H), 3.64-3.42 (m, 3H), 3.01-2.88 (m, 3H), 2.32-2.08 (m, 2H), 1.17 (d, J=6.9 Hz, 3H).

### Example 28: 4-((S)-1-((S)-1-((1-(4-chloro-2,5-difluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(4-chloro-2,5-difluorobenzyl)-1H-imidazol-4-yl)amino)-1-oxopropan-2-yl)-4,4-difluoropiperidin-3-yl)pyridine 1-oxide

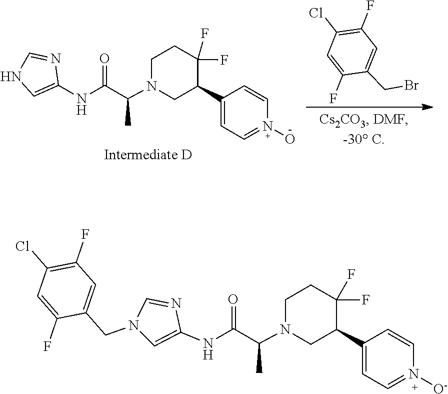

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.13 mmol, 1.5 equiv) in DMF (0.5 mL) was added 1-(bromomethyl)-4-chloro-2,5-difluorobenzene (27 mg, 0.11 mmol, 1.3 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.95) to afford 4-((S)-1-((S)-1-((1-(4-chloro-2,5-difluorobenzyl)-1H-imidazol-4-yl)amino)-1-oxopropan-2-yl)-4,4-difluoropiperidin-3-yl)pyridine 1-oxide (Compound 45) (8.6 mg, 19% yield) as a white solid.

LCMS [M+H]⁺: 512.2

¹H NMR (300 MHz, Methanol-d₄) δ 8.29 (d, J=7.2 Hz, 2H), 7.64 (d, J=1.6 Hz, 1H), 7.60 (d, J=6.8 Hz, 2H), 7.53-7.40 (m, 1H), 7.33 (d, J=1.6 Hz, 1H), 7.30-7.19 (m, 1H), 5.26 (d, J=1.4 Hz, 2H), 3.74-3.49 (m, 2H), 3.07-2.95 (m, 3H), 2.72-2.54 (m, 1H), 2.43-2.09 (m, 2H), 1.34 (d, J=7.0 Hz, 3H).

### Example 29: 4-((S)-1-((S)-1-((1-(3,4-difluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3,4-difluorobenzyl)-1H-imidazol-4-yl)amino)-1-oxopropan-2-yl)-4,4-difluoropiperidin-3-yl)pyridine 1-oxide

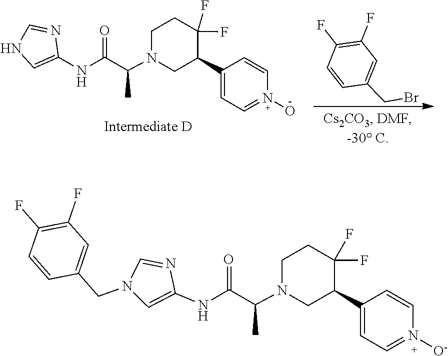

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (42 mg, 0.13 mmol, 1.5 equiv) in DMF (0.5 mL) were added 4-(bromomethyl)-1,2-difluorobenzene (19 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, Sum; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.65) to afford 4-((S)-1-((S)-1-((1-(3,4-difluorobenzyl)-1H-imidazol-4-yl)amino)-1-oxopropan-2-yl)-4,4-difluoropiperidin-3-yl)pyridine 1-oxide (Compound 46) (10 mg, 24% yield) as a white solid.

LCMS [M+H]⁺: 478.2

¹H NMR (400 MHz, Methanol-d₄) δ 8.29 (J=7.2 Hz, 2H), 7.66-7.53 (m, 3H), 7.28 (s, 1H), 7.26-7.18 (m, 2H), 7.15-7.09 (m, 1H), 5.17 (s, 2H), 3.78-3.46 (m, 2H), 3.12-3.03 (m, 2H), 2.96-2.90 (m, 1H), 2.72-2.54 (m, 1H), 2.41-2.09 (m, 2H), 1.32 (d, J=7.2 Hz, 3H).

### Example 30: 4-((S)-1-((S)-1-((1-(4-chloro-3-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(4-chloro-3-fluorobenzyl)-1H-imidazol-4-yl)amino)-1-oxopropan-2-yl)-4,4-difluoropiperidin-3-yl)pyridine 1-oxide

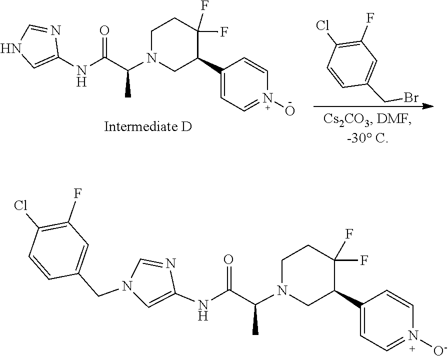

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (41 mg, 0.12 mmol, 1.5 equiv) in DMF (0.5 mL) were added 4-(bromomethyl)-1-chloro-2-fluorobenzene (23 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 1% B to 1% B in 1.5 min, 1% B to 15% B in 2 min, 15% B to 33% B in 9 min; Wave Length: 254 nm/220 nm; RT1 (min): 9.9) to afford 4-((S)-1-((S)-1-((1-(4-chloro-3-fluorobenzyl)-1H-imidazol-4-yl)amino)-1-oxopropan-2-yl)-4,4-difluoropiperidin-3-yl)pyridine 1-oxide (Compound 47) (11.4 mg, 26% yield) as a white solid.

LCMS [M+H]⁺: 494.2

¹H NMR (400 MHz, Methanol-d₄) δ 8.29 (J=7.2 Hz, 2H), 7.65-7.56 (m, 3H), 7.51-7.46 (m, 1H), 7.28 (d, J=1.6 Hz, 1H), 7.21-7.03 (m, 2H), 5.19 (s, 2H), 3.66-3.42 (m, 2H), 3.09-2.96 (m, 3H), 2.77-2.55 (m, 1H), 2.44-2.09 (m, 2H), 1.32 (d, J=7.0 Hz, 3H).

### Example 31: 4-((S)-1-((S)-1-((1-(2-chloro-4-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2-chloro-4-fluorobenzyl)-1H-imidazol-4-yl)amino)-1-oxopropan-2-yl)-4,4-difluoropiperidin-3-yl)pyridine 1-oxide

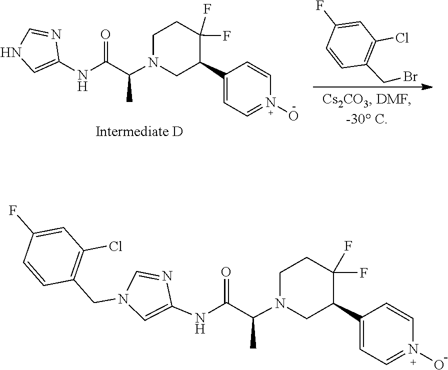

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (42 mg, 0.13 mmol, 1.5 equiv) in DMF (0.5 mL) was added 1-(bromomethyl)-2-chloro-4-fluorobenzene (21 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-1-((S)-1-((1-(2-chloro-4-fluorobenzyl)-1H-imidazol-4-yl)amino)-1-oxopropan-2-yl)-4,4-difluoropiperidin-3-yl)pyridine 1-oxide (Compound 48) (19.9 mg, 46.56% yield) as a white solid.

LCMS [M+H]⁺: 494.1

¹H NMR (400 MHz, DMSO-d₆) δ 10.22 (s, 1H), 8.16 (d, J=7.2 Hz, 2H), 7.60 (d, J=1.6 Hz, 1H), 7.58-7.49 (m, 1H), 7.40-7.35 (m, 3H), 7.32-7.25 (m, 1H), 7.23 (d, J=1.6 Hz, 1H), 5.24 (s, 2H), 3.62-3.46 (m, 3H), 3.09-2.75 (m, 3H), 2.23-2.03 (m, 2H), 1.16 (d, J=6.9 Hz, 3H).

### Example 32: 4-((S)-1-((S)-1-((1-(2,3-difluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2,3-difluorobenzyl)-1H-imidazol-4-yl)amino)-1-oxopropan-2-yl)-4,4-difluoropiperidin-3-yl)pyridine 1-oxide

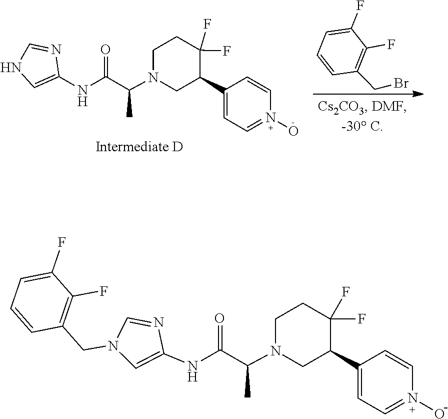

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.13 mmol, 1.5 equiv) in DMF (0.5 mL) was added 1-(bromomethyl)-2,3-difluorobenzene (20 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-1-((S)-1-((1-(2,3-difluorobenzyl)-1H-imidazol-4-yl)amino)-1-oxopropan-2-yl)-4,4-difluoropiperidin-3-yl)pyridine 1-oxide (Compound 49) (20.5 mg, 49% yield) as a white solid.

LCMS [M+H]⁺: 478.1

¹H NMR (400 MHz, DMSO-d₆) δ 10.22 (s, 1H), 8.16 (d, J=6.8 Hz, 2H), 7.61 (d, J=1.5 Hz, 1H), 7.48-7.39 (m, 1H), 7.37 (d, J=6.7 Hz, 2H), 7.29-7.20 (m, 2H), 7.18-7.10 (m, 1H), 5.28 (s, 2H), 3.62 (m, 3H), 3.03-2.79 (m, 3H), 2.18-1.99 (m, 2H), 1.17 (d, J=6.9 Hz, 3H).

### Example 33: 4-((S)-1-((S)-1-((1-(3,5-dichlorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3,5-dichlorobenzyl)-1H-imidazol-4-yl)amino)-1-oxopropan-2-yl)-4,4-difluoropiperidin-3-yl)pyridine 1-oxide

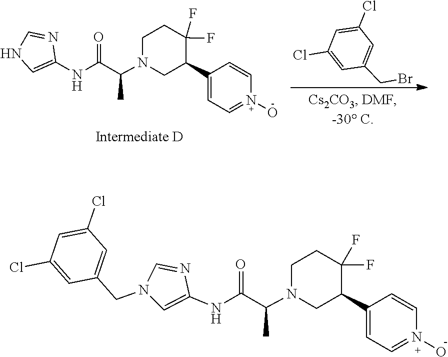

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol, 1 equiv) and Cs₂CO₃ (42 mg, 0.13 mmol, 1.5 equiv) in DMF (0.5 mL) was added 1-(bromomethyl)-3,5-dichlorobenzene (23 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-1-((S)-1-((1-(3,5-dichlorobenzyl)-1H-imidazol-4-yl)amino)-1-oxopropan-2-yl)-4,4-difluoropiperidin-3-yl)pyridine 1-oxide (Compound 50) (14.7 mg, 33% yield) as a white solid.

LCMS [M+H]⁺: 510.1

¹H NMR (400 MHz, DMSO-d₆) δ 10.21 (s, 1H), 8.16 (d, J=7.2 Hz, 2H), 7.66 (s, 1H), 7.58 (s, 1H), 7.41 (d, J=2.0 Hz, 2H), 7.36 (d, J=6.7 Hz, 2H), 7.31 (d, J=1.5 Hz, 1H), 5.15 (s, 2H), 3.55 (q, J=6.9 Hz, 1H), 3.48-3.41 (m, 2H), 3.03-2.88 (m, 3H), 2.26-2.03 (m, 2H), 1.17 (d, J=6.9 Hz, 3H).

### Example 34: 4-((S)-1-((S)-1-((1-(2-chloro-3-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2-chloro-3-fluorobenzyl)-1H-imidazol-4-yl)amino)-1-oxopropan-2-yl)-4,4-difluoropiperidin-3-yl)pyridine 1-oxide

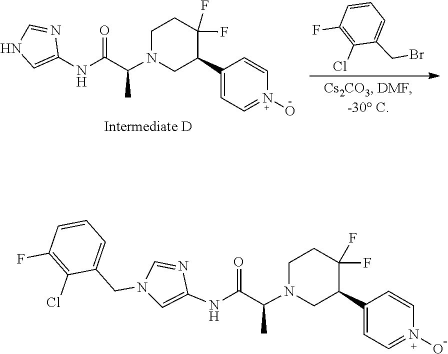

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs2CO3 (41 mg, 0.12 mmol, 1.5 equiv) in DMF (0.5 mL) was added 1-(bromomethyl)-2-chloro-3-fluorobenzene (23 mg, 0.10 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: Sunfire prep C18 column 30×150 mm, 5 μm; Mobile Phase A: Water (0.1% FA), Mobile Phase B: ACN; Flow rate: 60 mL/min; Gradient: 15% B to 35% B in 9 min; Wave Length: 254 nm/220 nm; RT1 (min): 9.65) to afford 4-((S)-1-((S)-1-((1-(2-chloro-3-fluorobenzyl)-1H-imidazol-4-yl)amino)-1-oxopropan-2-yl)-4,4-difluoropiperidin-3-yl)pyridine 1-oxide (Compound 51) (17.8 mg, 41% yield) as a light yellow solid.

LCMS [M+H]⁺: 494.2

¹H NMR (400 MHz, DMSO-d₆) δ 10.22 (s, 1H), 8.17 (d, J=7.0 Hz, 2H), 7.63 (d, J=1.5 Hz, 1H), 7.47-7.34 (m, 4H), 7.24 (d, J=1.6 Hz, 1H), 7.13-7.06 (m, 1H), 5.31 (s, 2H), 3.67-3.42 (m, 3H), 2.99-2.85 (m, 3H), 2.26-2.03 (m, 2H), 1.17 (d, J=6.9 Hz, 3H).

### Example 35: 4-((S)-1-((S)-1-((1-((3,5-difluorophenyl)methyl-d2)-1H-imidazol-4-yl)amino)-1-oxopropan-2-yl)-4,4-difluoropiperidin-3-yl)pyridine 1-oxide

### Step 1: (3,5-difluorophenyl)methan-d₂-ol-d

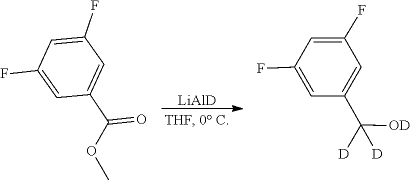

To a mixture of methyl 3,5-difluorobenzoate (500 mg, 2.90 mmol, 1 equiv) in THF (5 mL) was added lithium aluminum deuteride (2.9 mL, 1M in THF) dropwise at 0° C. under nitrogen atmosphere. The reaction was stirred at 0° C. for 1 h. To quench the reaction solid sodium sulfate decahydrate was slowly added until no bubbles were present. The resulting mixture was filtered, and the filter cake was washed with THF (5 mL). The filtrate was concentrated under reduced pressure to afford (3,5-difluorophenyl)methan-d₂-ol-d (270 mg, 63% yield) as a colorless oil. LCMS (ESI) [M+H]⁺: 148.

### Step 2: 1-(bromomethyl-d₂)-3,5-difluorobenzene

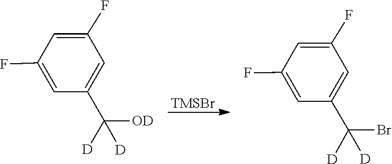

A mixture of (3,5-difluorophenyl)methan-d₂-ol-d (200 mg, 1.36 mmol, 1 equiv) and bromotrimethylsilane (312 mg, 2.04 mmol, 1.5 equiv) was stirred for 2 h at room temperature under nitrogen atmosphere. The reaction was quenched with water at 0° C. The resulting mixture was extracted with EtOAc (3×6 mL). The combined organic layers were washed with brine (3×8 mL), dried over anhydrous Na₂SO₄. The resulting mixture was concentrated under vacuum. This resulted in 1-(bromomethyl-d₂)-3,5-difluorobenzene (134 mg, 47% yield) as a colorless oil. LCMS (ESI) [M+H]⁺: 209.

### Step 3: 4-((S)-1-((S)-1-((1-((3,5-difluorophenyl)methyl-d2)-1H-imidazol-4-yl)amino)-1-oxopropan-2-yl)-4,4-difluoropiperidin-3-yl)pyridine 1-oxide

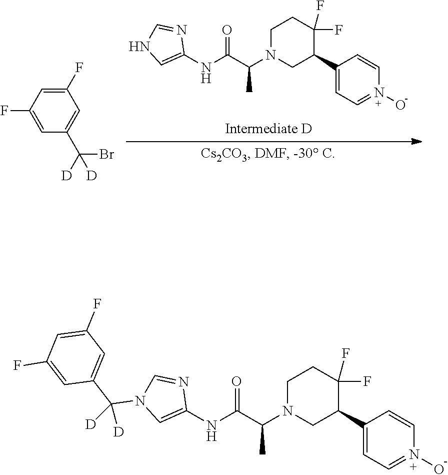

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol, 1 equiv) and Cs₂CO₃ (42 mg, 0.13 mmol, 1.5 equiv) in DMF (0.5 mL) was added 1-(bromomethyl-d₂)-3,5-difluorobenzene (19 mg, 0.09 mmol, 1.1 equiv) dropwise at −30° C. under nitrogen atmosphere. The resulting mixture was stirred for 2 h at −30° C. under nitrogen atmosphere. The resulting mixture was filtered, and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 um; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.65) to afford 4-((S)-1-((S)-1-((1-((3,5-difluorophenyl)methyl-d₂)-1H-imidazol-4-yl)amino)-1-oxopropan-2-yl)-4,4-difluoropiperidin-3-yl)pyridine 1-oxide (Compound 52) (8.5 mg, 20% yield assumed) as a white solid.

LCMS [M+H]⁺: 480.2

¹H NMR (400 MHz, DMSO-d₆) δ 10.19 (s, 1H), 8.17 (d, J=7.2 Hz, 2H), 7.64 (d, J=1.6 Hz, 1H), 7.36 (d, J=6.8 Hz, 2H), 7.30 (d, J=1.6 Hz, 1H), 7.24-7.16 (m, 1H), 7.10-7.03 (m, 2H), 3.66-3.48 (m, 3H), 3.03-2.81 (m, 3H), 2.26-2.08 (m, 2H), 1.17 (d, J=6.8 Hz, 3H).

### Example 36: 4-((S)-1-((S)-1-((1-(3-cyano-5-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3-cyano-5-fluorobenzyl)-1H-imidazol-4-yl)amino)-1-oxopropan-2-yl)-4,4-difluoropiperidin-3-yl)pyridine 1-oxide

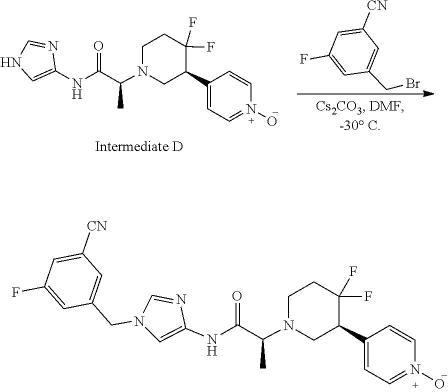

To a solution of 4-((S)-1-((S)-1-((1H-imidazol-4-yl)amino)-1-oxopropan-2-yl)-4,4-difluoropiperidin-3-yl)pyridine 1-oxide (Intermediate D) (30 mg, 0.08 mmol, 1 equiv) and Cs₂CO₃ (41 mg, 0.12 mmol, 1.5 equiv) in DMF (0.5 mL) was added 3-(bromomethyl)-5-fluorobenzonitrile (20 mg, 0.09 mmol, 1.1 equiv) dropwise at −30° C. under nitrogen atmosphere. The resulting mixture was stirred at −30° C. for 2 h. The resulting mixture was filtered, and the filter cake was washed with DMF (0.5 mL). The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-1-((S)-1-((1-(3-cyano-5-fluorobenzyl)-1H-imidazol-4-yl)amino)-1-oxopropan-2-yl)-4,4-difluoropiperidin-3-yl)pyridine 1-oxide (Compound 53) (7.1 mg, 16% yield) as a yellow solid.

LCMS [M+H]⁺: 485.1

¹H NMR (400 MHz, DMSO-d₆) δ 10.20 (s, 1H), 8.17 (d, J=6.7 Hz, 2H), 7.83 (d, J=8.6 Hz, 1H), 7.71 (s, 1H), 7.66 (d, J=1.6 Hz, 1H), 7.59 (d, J=9.1 Hz, 1H), 7.42-7.27 (m, 3H), 5.21 (s, 2H), 3.67-3.42 (m, 3H), 3.03-2.80 (m, 3H), 2.23-1.96 (m, 2H), 1.17 (d, J=6.8 Hz, 3H).

### Example 37: 2-((S)-4,4-difluoro-3-(6-oxo-1,6-dihydropyridin-3-yl)piperidin-1-yl)-N-(1-(3,5-difluorobenzyl)-1H-imidazol-4-yl)propanamide

### Step 1: tert-butyl 3-(6-methoxypyridin-3-yl)-4-oxopiperidine-1-carboxylate

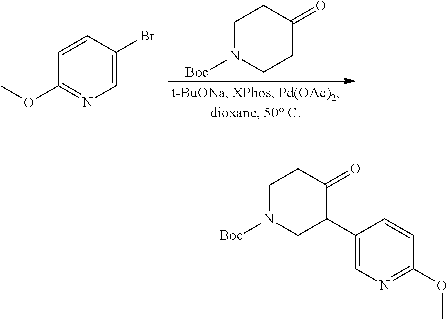

A mixture of 5-bromo-2-methoxypyridine (1.0 g, 5.31 mmol, 1 equiv), tert-butyl 4-oxopiperidine-1-carboxylate (1.3 g, 6.52 mmol, 1.2 equiv), t-BuONa (1.5 g, 15.60 mmol, 3 equiv), XPHOS (0.5 g, 1.04 mmol, 0.2 equiv) and Pd(OAc)₂ (0.1 g, 0.44 mmol, 0.1 equiv) in dioxane (20 mL) was stirred for 1 h at 50° C. under nitrogen atmosphere. The mixture was allowed to cool down to room temperature. The resulting mixture was filtered, and the filter cake was washed with EtOAc (3×25 mL). The resulting mixture was concentrated under reduced pressure. The residue was purified by silica gel column chromatography, eluted with CH₂Cl₂/EA (3:1) to afford tert-butyl 3-(6-methoxypyridin-3-yl)-4-oxopiperidine-1-carboxylate (500 mg, 30% yield) as a yellow oil. LCMS (ESI) [M+H]⁺: 307.

### Step 2: tert-butyl 4,4-difluoro-3-(6-methoxypyridin-3-yl)piperidine-1-carboxylate

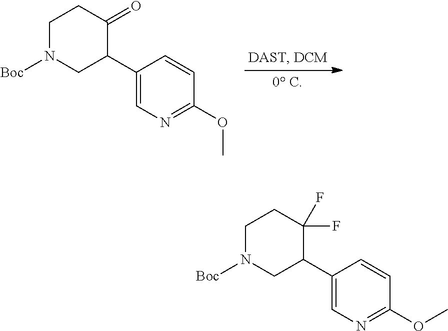 [SMILES: COc1ccc(C2CN(C(=O)OC(C)(C)C)CCC2=O)cn1 ; COc1ccc(C2CN(C(=O)OC(C)(C)C)CCC2(F)F)cn1]

To a mixture of tert-butyl 3-(6-methoxypyridin-3-yl)-4-oxopiperidine-1-carboxylate (490 mg, 1.60 mmol, 1 equiv) in DCM (5 mL) was added DAST (516 mg, 3.20 mmol, 2 equiv) dropwise at 0° C. under N₂ atmosphere. The final reaction mixture was stirred at room temperature for 3 h. The reaction was quenched by the addition of sat. sodium hyposulfite (aq.) (12 mL) at 0° C. The resulting mixture was extracted with EtOAc (3×15 mL). The combined organic layers were dried over anhydrous Na₂SO₄. The resulting mixture was concentrated under reduced pressure. The residue was purified by silica gel column chromatography, eluted with PE/EA (1:1) to afford tert-butyl 4,4-difluoro-3-(6-methoxypyridin-3-yl)piperidine-1-carboxylate (300 mg, 57% yield) as a white solid. LCMS (ESI) [M+H]⁺: 329.

### Step 3: 5-(4,4-difluoropiperidin-3-yl)pyridin-2(1H)-one

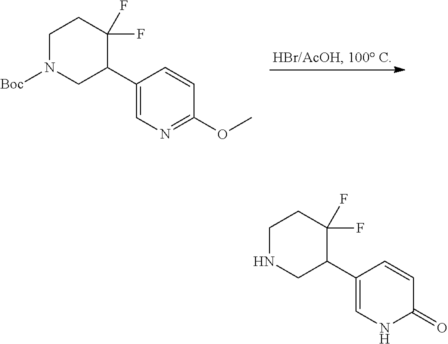 [SMILES: COc1ccc(C2CN(C(=O)OC(C)(C)C)CCC2(F)F)cn1 ; O=c1ccc(C2CNCCC2(F)F)c[nH]1]

A solution of tert-butyl 4,4-difluoro-3-(6-methoxypyridin-3-yl)piperidine-1-carboxylate (300 mg, 0.91 mmol, 1 equiv) in HBr/AcOH (1 mL) was stirred for 1 h at 100° C. under N₂ atmosphere. The mixture was allowed to cool down to room temperature. The resulting mixture was concentrated under vacuum. The residue was purified by silica gel column chromatography, eluted with CH₂Cl₂/MeOH (1:1) to 5-(4,4-difluoropiperidin-3-yl)pyridin-2(1H)-one (150 mg, 76% yield) as a yellow oil. LCMS (ESI) [M+H]⁺: 215

### Step 4: (S)-5-(4,4-difluoropiperidin-3-yl)pyridin-2(1H)-one

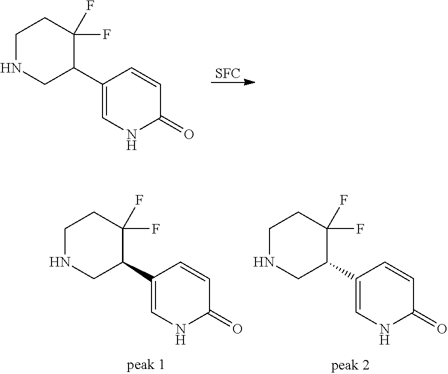

5-(4,4-difluoropiperidin-3-yl)pyridin-2(1H)-one (150 mg) was separated by prep-Chiral-HPLC (Column: CHIRALPAK IG, 3×25 cm, 5 μm; Mobile Phase A: Hex (10 mM NH₃-MeOH), Mobile Phase B: IPA; Flow rate: 40 mL/min; Gradient: isocratic 30% B; Wave Length: 214/303 nm; RT1 (min): 13.2; RT2 (min): 19.3; Sample Solvent: MeOH:DCM=1:1; Number Of Runs: 4) to afford (S)-5-(4,4-difluoropiperidin-3-yl)pyridin-2(1H)-one (60 mg, 100% e.e.) and (R)-5-(4,4-difluoropiperidin-3-yl)pyridin-2(1H)-one (70 mg, 100% e.e.). LCMS (ESI) [M+H]⁺: 215.

### Step 5: 2-((S)-4,4-difluoro-3-(6-oxo-1,6-dihydropyridin-3-yl)piperidin-1-yl)-N-(1-(3,5-difluorobenzyl)-1H-imidazol-4-yl)propanamide

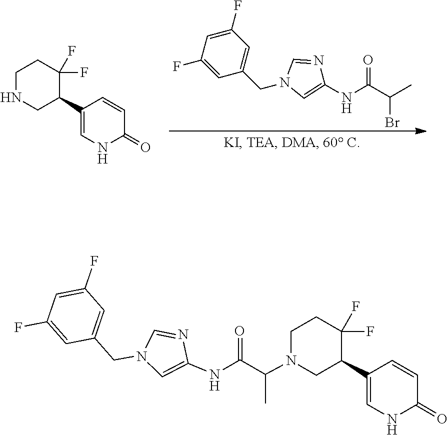 [SMILES: O=c1ccc([C@H]2CNCCC2(F)F)c[nH]1 ; CC(Br)C(=O)Nc1cn(Cc2cc(F)cc(F)c2)cn1 ; CC(C(=O)Nc1cn(Cc2cc(F)cc(F)c2)cn1)N1CCC(F)(F)[C@@H](c2ccc(=O)[nH]c2)C1]

A solution of (S)-5-(4,4-difluoropiperidin-3-yl)pyridin-2(1H)-one (60 mg, 0.28 mmol, 1 equiv), 2-bromo-N-(1-(3,5-difluorobenzyl)-1H-imidazol-4-yl)propanamide (116 mg, 0.33 mmol, 1.2 equiv), KI (56 mg, 0.33 mmol, 1.2 equiv) and TEA (85 mg, 0.84 mmol, 3 equiv) in DMA (1 mL) was stirred for 1 h at 60° C. under N₂ atmosphere. The mixture was allowed to cool down to room temperature. The residue was dissolved in water (3 mL). The resulting mixture was extracted with EtOAc (3×5 mL). The combined organic layers were washed with water (3×6 mL), dried over anhydrous Na₂SO₄. The resulting mixture was concentrated under vacuum. The crude product was purified by Prep-HPLC with the following conditions (Column: XBridge Shield RP18 OBD Column 30×150 mm, 5 μm; Mobile Phase A: Water (10 mmol/L NH₄HCO₃), Mobile Phase B: ACN; Flow rate: 60 mL/min; Gradient: 5% B to 5% B in 1.5 min, 5% B to 20% B in 2 min, 20% B to 41% B in 9 min; Wave Length: 254 nm/220 nm; RT1 (min): 10.07) to afford 2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (70 mg, 52% yield) as a white solid. LCMS (ESI) [M+H]⁺: 478.

### Step 6: (S)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide and (R)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide

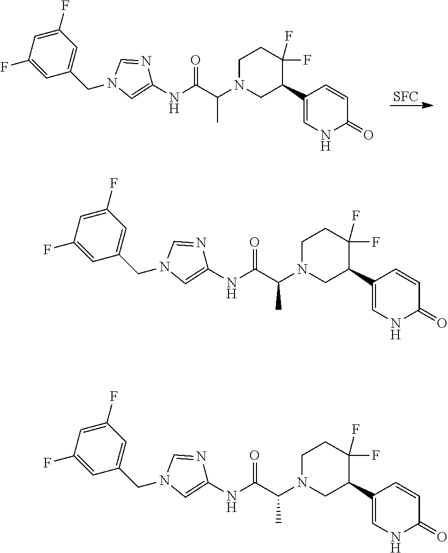

2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (70 mg) was separated by prep-Chiral-HPLC (Column: CHIRALPAK IA, 3×25 cm, 5 μm; Mobile Phase A: Hex (10 mM NH₃-MeOH), Mobile Phase B: EtOH; Flow rate: 40 mL/min; Gradient: isocratic 50% B; Wave Length: 210/220 nm; RT1 (min): 9.5; RT2 (min): 20.3; Sample Solvent: ETOH:DCM=1:1; Injection Volume: 2 mL; Number Of Runs: 2) to afford (S)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (Compound 54) (Method L, 1.01 min, peak 1, 30.2 mg, 100% e.e.) and (R)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (Compound 55) (Method L, 1.75 min, peak 2, 23.3 mg, 100% e.e.).

TABLE 15

| Compound# | Characterization Data |
|---|---|
| 54 | LCMS (ESI) [M + H]⁺: 478.3 |
| | ¹H NMR (400 MHz, DMSO-d₆) δ 11.54 (s, 1H), 10.17 (s, 1H), 7.63 (s, 1H), 7.38 (d, J = 9.5 |
| | Hz, 1H), 7.30 (d, J = 4.6 Hz, 2H), 7.23 − 7.16 (m, 1H), 7.06 (d, J = 7.3 Hz, 2H), 6.27 (d, J = |
| | 9.4 Hz, 1H), 5.16 (s, 2H), 3.52 − 3.49 (m, 1H), 3.23 − 3.13 (m, 1H), 3.01 − 2.82 (m, 2H), 2.63 − |
| | 58 (m, 2H), 2.16 − 1.89 (m, 2H), 1.17 (d, J = 6.8 Hz, 3H). |
| 55 | LCMS (ESI) [M + H]⁺: 478.2 |
| | ¹H NMR (400 MHz, DMSO-d₆) δ 11.57 (s, 1H), 10.16 (s, 1H), 7.64 (s, 1H), 7.39 (d, J = 9.5 |
| | Hz, 1H), 7.32 − 7.26 (m, 2H), 7.22 − 7.16 (m, 1H), 7.06 (d, J = 7.2 Hz, 2H), 6.29 (d, J = 9.5 |
| | Hz, 1H), 5.17 (s, 2H), 3.56 − 3.51 (m, 1H), 3.21 − 3.07 (m, 1H), 2.89 − 2.71 (m, 3H), 2.49 − |
| | 2.41 (s, 1H), 2.13 − 1.95 (m, 2H), 1.16 (d, J = 6.9 Hz, 3H). |

### Example 38: 4-((3S)-1-(1-((5-(3,5-difluorobenzyl)-1,2,4-oxadiazol-3-yl)amino)-1-oxopropan-2-yl)-4,4-difluoropiperidin-3-yl)pyridine 1-oxide

### Step 1: N-cyano-2-(3,5-difluorophenyl)acetamide

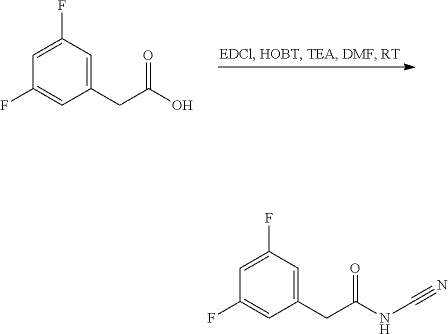 [SMILES: O=C(O)Cc1cc(F)cc(F)c1 ; N#CNC(=O)Cc1cc(F)cc(F)c1]

A solution of (3,5-difluorophenyl)acetic acid (2.0 g, 11.61 mmol, 1 equiv), hydroxybenzotriazole or HOBT (0.8 g, 5.80 mmol, 0.5 equiv), TEA (3.5 g, 34.85 mmol, 3 equiv) and 1-Ethyl-3-(3-dimethylaminopropyl)carbodiimide or EDCI (3.3 g, 17.42 mmol, 1.5 equiv) in DMF (20 mL) was stirred for 2 h at room temperature under nitrogen atmosphere. The reaction was quenched with water (25 mL) at room temperature. The resulting mixture was extracted with EtOAc (3×30 mL). The combined organic layers were washed with water (2×35 mL), dried over anhydrous Na₂SO₄. The resulting mixture was concentrated under reduced pressure. The residue was purified by reversed-phase flash chromatography with the following conditions: column, C18 silica gel; mobile phase, MeCN in Water (10 mmol/L NH₄HCO₃), 0% to 100% gradient in 30 min; detector, UV 254 nm and UV 220 nm to afford N-cyano-2-(3,5-difluorophenyl)acetamide (1.8 g, 78% yield) as a white solid. LCMS [M+H]⁺: 197.

### Step 2: 5-(3,5-difluorobenzyl)-1,2,4-oxadiazol-3-amine

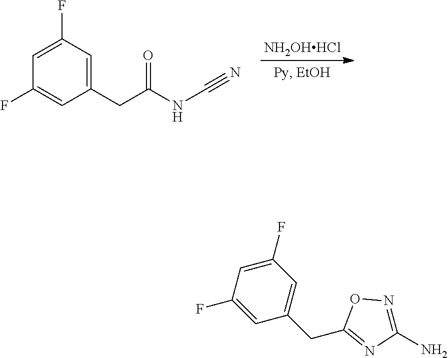

A solution of N-cyano-2-(3,5-difluorophenyl)acetamide (1.0 g, 5.09 mmol, 1 equiv), hydroxylamine hydrochloride (0.5 g, 7.65 mmol, 1.5 equiv) and pyridine (1.2 g, 15.29 mmol, 3 equiv) in EtOH (10 mL) was stirred for overnight at room temperature under nitrogen atmosphere. The reaction was quenched by the addition of water (20 mL) at room temperature. The resulting mixture was extracted with EtOAc (3×25 mL). The combined organic layers were dried over anhydrous Na₂SO₄. The resulting mixture was concentrated under reduced pressure. The residue was purified by reversed-phase flash chromatography with the following conditions: column, C18; mobile phase, MeCN in Water (10 mmol/L NH₄HCO₃), 0% to 100% gradient in 25 min; detector, UV 254 nm and UV 220 nm to afford 5-(3,5-difluorobenzyl)-1,2,4-oxadiazol-3-amine (260 mg, 24% yield) as a white oil. LCMS [M+H]⁺: 212.

### Step 3: 2-bromo-N-(5-(3,5-difluorobenzyl)-1,2,4-oxadiazol-3-yl)propanamide

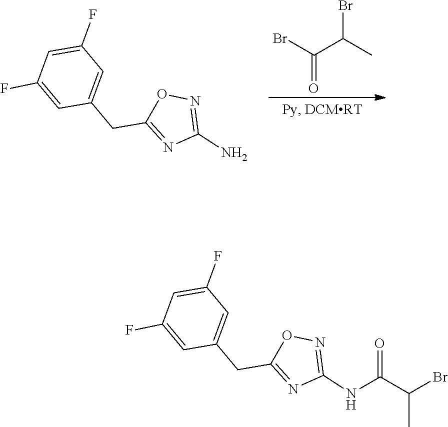

A solution of 5-(3,5-difluorobenzyl)-1,2,4-oxadiazol-3-amine (250 mg, 1.18 mmol, 1 equiv) and pyridine (281 mg, 3.55 mmol, 3 equiv) in DCM (3 mL) was treated with 2-bromopropanoyl bromide (383 mg, 1.77 mmol, 1.5 equiv) at 0° C. under nitrogen atmosphere. The final reaction mixture was stirred at room temperature for 1 h. The reaction was quenched by the addition of water (5 mL) at 0° C. The resulting mixture was extracted with EtOAc (3×8 mL). The combined organic layers were dried over anhydrous Na₂SO₄. The resulting mixture was concentrated under reduced pressure. The residue was purified by silica gel column chromatography, eluted with PE/EA (1:10) to afford 2-bromo-N-(5-(3,5-difluorobenzyl)-1,2,4-oxadiazol-3-yl)propanamide (190 mg, 46% yield) as a white solid. LCMS [M+H]⁺: 346.

### Step 4: 4-((3S)-1-(1-((5-(3,5-difluorobenzyl)-1,2,4-oxadiazol-3-yl)amino)-1-oxopropan-2-yl)-4,4-difluoropiperidin-3-yl)pyridine 1-oxide

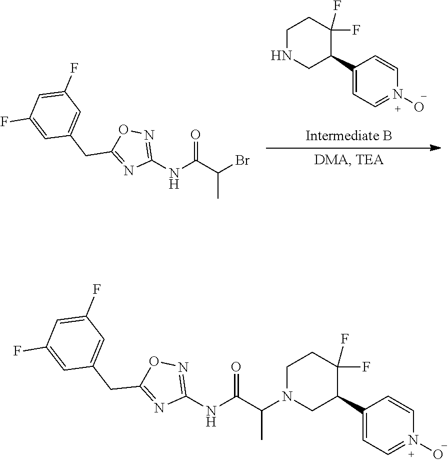

A solution of 2-bromo-N-(5-(3,5-difluorobenzyl)-1,2,4-oxadiazol-3-yl)propanamide (120 mg, 0.34 mmol, 1 equiv), TEA (175 mg, 1.73 mmol, 5 equiv) and (S)-4-(4,4-difluoropiperidin-3-yl)pyridine 1-oxide (Intermediate B) (74 mg, 0.34 mmol, 1 equiv) in DMA (2 mL) was stirred overnight at room temperature under nitrogen atmosphere. The crude product was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 17% B to 34% B in 10 min; Wave Length: 254 nm/220 nm; RT1 (min): 11.08) to afford 4-((3S)-1-(1-((5-(3,5-difluorobenzyl)-1,2,4-oxadiazol-3-yl)amino)-1-oxopropan-2-yl)-4,4-difluoropiperidin-3-yl)pyridine 1-oxide (50 mg, 30% yield) as a light yellow oil. LCMS [M+H]⁺: 480.

### Step 5: 4-((S)-1-((S)-1-((5-(3,5-difluorobenzyl)-1,2,4-oxadiazol-3-yl)amino)-1-oxopropan-2-yl)-4,4-difluoropiperidin-3-yl)pyridine 1-oxide and 4-((S)-1-((R)-1-((5-(3,5-difluorobenzyl)-1,2,4-oxadiazol-3-yl)amino)-1-oxopropan-2-yl)-4,4-difluoropiperidin-3-yl)pyridine 1-oxide

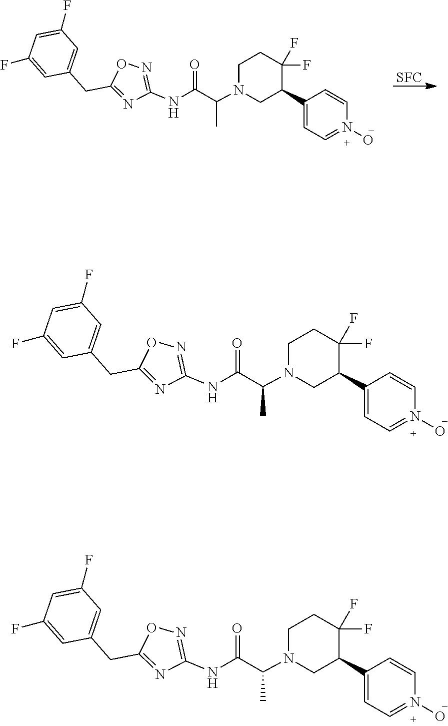

4-((3S)-1-(1-((5-(3,5-difluorobenzyl)-1,2,4-oxadiazol-3-yl)amino)-1-oxopropan-2-yl)-4,4-difluoropiperidin-3-yl)pyridine 1-oxide (50 mg) was separated by prep-Chiral-HPLC (Column: CHIRALPAK ID, 3×25 cm, 5 μm; Mobile Phase A: MtBE (0.1% FA), Mobile Phase B: MEOH; Flow rate: 40 mL/min; Gradient: isocratic 15% B; Wave Length: 212/274 nm; RT1 (min): 12.06; RT2 (min): 16.18; Sample Solvent: MEOH; Injection Volume: 1.3 mL; Number Of Runs: 1) to afford 4-((S)-1-((S)-1-((5-(3,5-difluorobenzyl)-1,2,4-oxadiazol-3-yl)amino)-1-oxopropan-2-yl)-4,4-difluoropiperidin-3-yl)pyridine 1-oxide (Compound 56) (Method M, 1.20 min, peak 1, 8.9 mg, 83.7% e.e.) and 4-((S)-1-((R)-1-((5-(3,5-difluorobenzyl)-1,2,4-oxadiazol-3-yl)amino)-1-oxopropan-2-yl)-4,4-difluoropiperidin-3-yl)pyridine 1-oxide (Compound 57) (Method M, 1.36 min, peak 2, 3.7 mg, 84.3% e.e.).

TABLE 16

| Compound# | Characterization Data |
|---|---|
| 56 | LCMS (ESI) [M + H]⁺: 480.1 |
| | ¹H NMR (400 MHz, Methanol-d₄) δ 8.29 (d, J = 7.2 Hz, 2H), 7.57 (d, J = 6.5 Hz, 2H), 7.03 − |
| | 6.94 (m, 2H), 6.92 − 6.83 (m, 1H), 4.36 − 4.31 (m, 1H), 3.80 (s, 2H), 3.50 − 3.40 (m, 1H), |
| | 3.17 − 3.13 (m, 1H), 3.02 − 2.99 (m, 1H), 2.87 − 2.73 (m, 2H), 2.20 − 2.06 (m, 2H), 1.56 (d, |
| | J = 7.1 Hz, 3H). |
| 57 | LCMS (ESI) [M + H]⁺: 480.2 |
| | ¹H NMR (400 MHz, Methanol-d₄) δ 8.29 (d, J = 6.3 Hz, 2H), 7.59 (d, J = 6.4 Hz, 2H), 6.97 |
| | (d, J = 7.5 Hz, 2H), 6.92 − 6.82 (m, 1H), 4.32 (q, J = 7.0 Hz, 1H), 3.80 (s, 2H), 3.59 − 3.40 |
| | (m, 1H), 3.15 − 2.96 (m, 3H), 2.69 − 2.46 (m, 1H), 2.25 − 2.09 (m, 2H), 1.56 (d, J = 7.0 Hz, |
| | 3H). |

### Example 39: 4-((3S)-1-(1-((3-(3,5-difluorobenzyl)-1,2,4-oxadiazol-5-yl)amino)-1-oxopropan-2-yl)-4,4-difluoropiperidin-3-yl)pyridine 1-oxide

### Step 1: 2-(3,5-difluorophenyl)acetonitrile

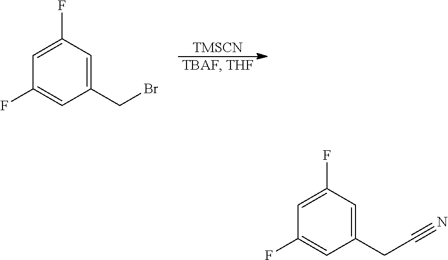

A solution of TMSCN (0.6 g, 5.79 mmol, 1.2 equiv) and TBAF (1.4 g, 5.31 mmol, 1.1 equiv) in THF (10 mL) was stirred for 1 h at 0° C. under N₂ atmosphere. To the above mixture was added 1-(bromomethyl)-3,5-difluorobenzene (1.0 g, 4.83 mmol, 1 equiv) dropwise at room temperature. The resulting mixture was stirred at 80° C. for an additional 1 h. The resulting mixture was concentrated under reduced pressure. The residue was purified by silica gel column chromatography, eluted with CH₂Cl₂/PE (1:1) to afford 2-(3,5-difluorophenyl)acetonitrile (530 mg, 71% yield) as a white solid. LCMS (ESI) [M+H]⁺: 154.

### Step 2: (Z)-2-(3,5-difluorophenyl)-N′-hydroxyacetimidamide

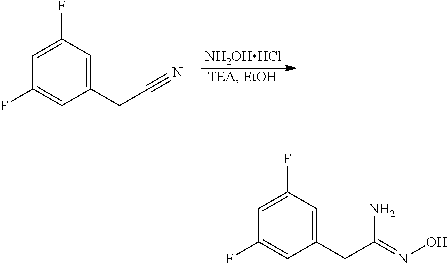

A solution of 2-(3,5-difluorophenyl)acetonitrile (520 mg, 3.39 mmol, 1 equiv) and hydroxylamine hydrochloride (283 mg, 4.07 mmol, 1.2 equiv) and TEA (859 mg, 8.48 mmol, 2.5 equiv) in EtOH (5 mL) was stirred at 60° C. overnight under nitrogen atmosphere. The resulting mixture was concentrated under reduced pressure. The residue was purified by silica gel column chromatography, eluted with CH₂Cl₂/EA (1:1) to afford (Z)-2-(3,5-difluorophenyl)-N-hydroxyacetimidamide (500 mg, 79% yield) as a white solid. LCMS (ESI) [M+H]⁺: 187.

### Step 3: 3-(3,5-difluorobenzyl)-1,2,4-oxadiazol-5-amine

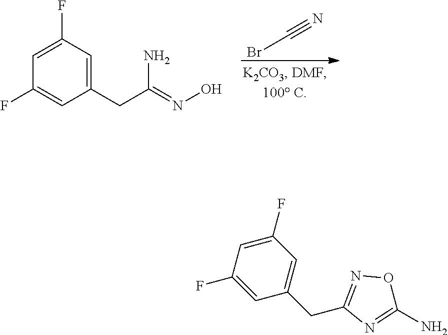

To a mixture of (Z)-2-(3,5-difluorophenyl)-N-hydroxyacetimidamide (500 mg, 2.69 mmol, 1 equiv) and K₂CO₃ (1.1 g, 8.05 mmol, 3 equiv) in DMF (5 mL) was added cyanogen bromide (341 mg, 3.21 mmol, 1.2 equiv) dropwise at 0° C. under N₂ atmosphere. The final reaction mixture was stirred at room temperature for 2 h. The solution was diluted with water (10 mL). The resulting mixture was extracted with EtOAc (3×15 mL). The combined organic layers were washed with water (3×20 mL), dried over anhydrous Na₂SO₄. The resulting mixture was concentrated under reduced pressure. The residue was purified by silica gel column chromatography, eluted with PE/EA (1:2) to afford 3-(3,5-difluorobenzyl)-1,2,4-oxadiazol-5-amine (145 mg, 25% yield) as a white solid. LCMS (ESI) [M+H]⁺: 212.

### Step 4: 2-bromo-N-(3-(3,5-difluorobenzyl)-1,2,4-oxadiazol-5-yl)propanamide

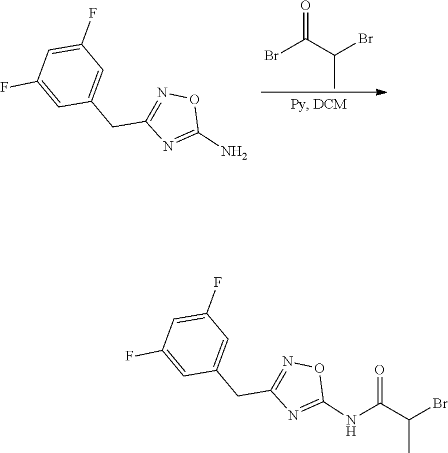

To a mixture of 3-(3,5-difluorobenzyl)-1,2,4-oxadiazol-5-amine (145 mg, 0.68 mmol, 1 equiv) and pyridine (163 mg, 2.06 mmol, 3 equiv) in DCM (2 mL) were added 2-bromopropanoyl bromide (296 mg, 1.37 mmol, 2 equiv) dropwise at 0° C. under N₂ atmosphere. The reaction was quenched with water at 0° C. The resulting mixture was extracted with EtOAc (3×5 mL). The combined organic layers were washed with water (3×7 mL), dried over anhydrous Na₂SO₄. The resulting mixture was concentrated under reduced pressure. The residue was purified by silica gel column chromatography, eluted with PE/EA (1:1) to afford 2-bromo-N-(3-(3,5-difluorobenzyl)-1,2,4-oxadiazol-5-yl)propanamide (150 mg, 63% yield) as a white solid. LCMS (ESI) [M+H]⁺: 346.

### Step 5: 4-((3S)-1-(1-((3-(3,5-difluorobenzyl)-1,2,4-oxadiazol-5-yl)amino)-1-oxopropan-2-yl)-4,4-difluoropiperidin-3-yl)pyridine 1-oxide

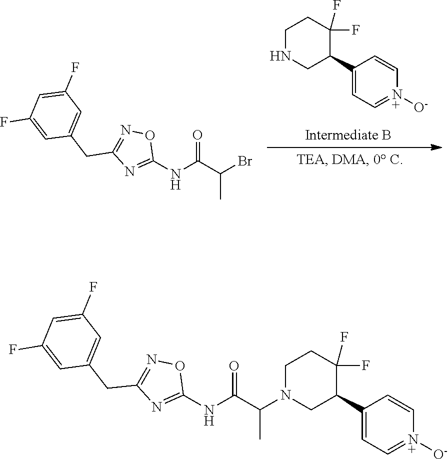 [SMILES: CC(Br)C(=O)Nc1nc(Cc2cc(F)cc(F)c2)no1 ; [O-][n+]1ccc([C@H]2CNCCC2(F)F)cc1 ; CC(C(=O)Nc1nc(Cc2cc(F)cc(F)c2)no1)N1CCC(F)(F)[C@@H](c2cc[n+]([O-])cc2)C1]

A solution of 2-bromo-N-(3-(3,5-difluorobenzyl)-1,2,4-oxadiazol-5-yl)propanamide (150 mg, 0.43 mmol, 1 equiv) and (S)-4-(4,4-difluoropiperidin-3-yl)pyridine 1-oxide (Intermediate B) (111 mg, 0.51 mmol, 1.2 equiv) and TEA (132 mg, 1.3 mmol, 3 equiv) in DMA (2 mL) was stirred at room temperature overnight under air atmosphere. The crude product was purified by Prep-HPLC with the following conditions (Column: XBridge Shield RP18 OBD Column 30×150 mm, 5 μm; Mobile Phase A: Water (10 mmol/L NH₄HCO₃), Mobile Phase B: ACN; Flow rate: 60 mL/min; Gradient: 5% B to 5% B in 1.5 min, 5% B to 20% B in 2 min, 20% B to 41% B in 9 min; Wave Length: 254 nm/220 nm; RT1 (min): 9.07) to afford 4-((3S)-1-(1-((3-(3,5-difluorobenzyl)-1,2,4-oxadiazol-5-yl)amino)-1-oxopropan-2-yl)-4,4-difluoropiperidin-3-yl)pyridine 1-oxide (75 mg, 36% yield) as a white solid. LCMS (ESI) [M+H]⁺: 480.

### Step 6: 4-((S)-1-((S)-1-((3-(3,5-difluorobenzyl)-1,2,4-oxadiazol-5-yl)amino)-1-oxopropan-2-yl)-4,4-difluoropiperidin-3-yl)pyridine 1-oxide and 4-((S)-1-((R)-1-((3-(3,5-difluorobenzyl)-1,2,4-oxadiazol-5-yl)amino)-1-oxopropan-2-yl)-4,4-difluoropiperidin-3-yl)pyridine 1-oxide

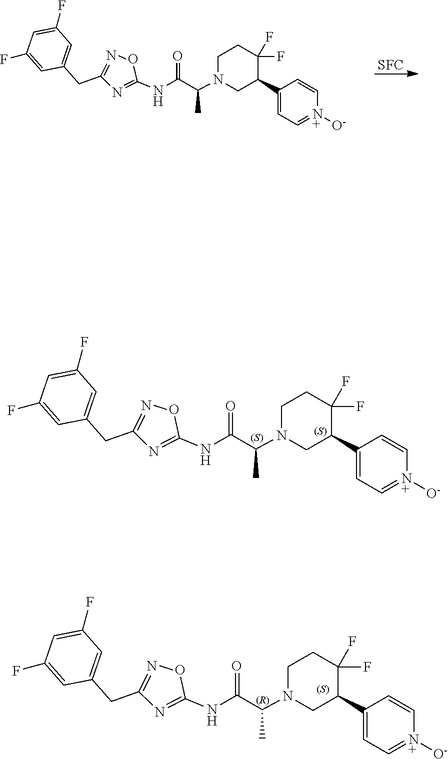

4-((3S)-1-(1-((3-(3,5-difluorobenzyl)-1,2,4-oxadiazol-5-yl)amino)-1-oxopropan-2-yl)-4,4-difluoropiperidin-3-yl)pyridine 1-oxide (75 mg) was separated by prep-Chiral-HPLC (Column: CHIRAL ART Cellulose-SB, 3×25 cm, 5 μm; Mobile Phase A: MtBE (0.1% FA), Mobile Phase B: MEOH; Flow rate: 20 mL/min; Gradient: isocratic 15% B; Wave Length: 214/274 nm; RT1 (min): 8.03; RT2 (min): 10.22; Sample Solvent: MEOH; Injection Volume: 0.5 mL; Number Of Runs: 4) to afford 4-((S)-1-((S)-1-((3-(3,5-difluorobenzyl)-1,2,4-oxadiazol-5-yl)amino)-1-oxopropan-2-yl)-4,4-difluoropiperidin-3-yl)pyridine 1-oxide (Compound 58) (Method N, 1.03 min, peak 1, 25.2 mg, 100% e.e.) and 4-((S)-1-((R)-1-((3-(3,5-difluorobenzyl)-1,2,4-oxadiazol-5-yl)amino)-1-oxopropan-2-yl)-4,4-difluoropiperidin-3-yl)pyridine 1-oxide (Compound 59) (Method N, 1.30 min, peak 2, 21.2 mg, 99% e.e.).

TABLE 17

| Compound# | Characterization Data |
|---|---|
| 58 | LCMS (ESI) [M + H]⁺: 480.2 |
| | ¹H NMR (400 MHz, Methanol-d₄) δ 8.27 (d, J = 7.2 Hz, 2H), 7.54 (d, J = 6.5 Hz, 2H), 6.96 (d, J = |
| | 7.4 Hz, 2H), 6.87 − 6.70 (m, 1H), 4.03 (s, 2H), 3.68 − 3.59 (m, 1H), 3.54 − 3.38 (m, 1H), 3.02 |
| | (m, 4H), 2.26 − 2.08 (m, 2H), 1.33 (d, J = 6.9 Hz, 3H). |
| 59 | LCMS (ESI) [M + H]⁺: 480.2 |
| | ¹H NMR (400 MHz, Methanol-d₄) δ 8.29 (d, J = 7.2 Hz, 2H), 7.56 (d, J = 6.5 Hz, 2H), 7.01 − |
| | 6.93 (m, 2H), 6.86 − 6.81 (m, 1H), 4.04 (s, 2H), 3.65 − 3.51 (m, 2H), 3.05 (d, J = 8.0 Hz, 2H), |
| | 2.89 − 2.83 (m, 1H), 2.72 − 2.66 (m, 1H), 2.19 − 2.11 (m, 2H), 1.32 (d, J = 6.9 Hz, 3H). |

### Example 40: 4-((3S)-1-(1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide

### Step 1: methyl 4-bromo-1-(3,5-difluorobenzyl)-1H-imidazole-2-carboxylate

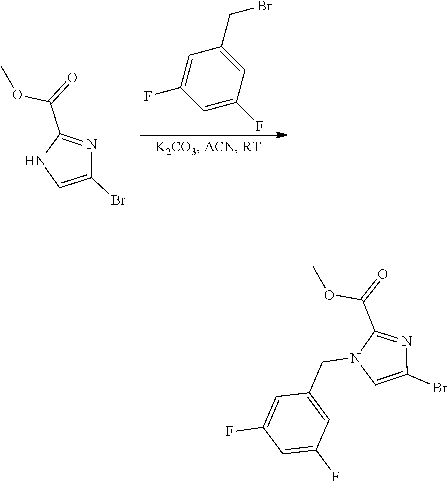

A mixture of methyl 4-bromo-1H-imidazole-2-carboxylate (5.0 g, 24.4 mmol), 1-(1-bromoethyl)-3,5-difluorobenzene (5.1 g, 24.4 mmol), K₂CO₃ (10.1 g, 72 mmol) in ACN (20 mL) was stirred for 2 hours at 80° C. The resulting mixture was concentrated under reduced pressure. The residue was purified by silica gel column chromatography, eluted with CH₂Cl₂/PE (9:1) to afford methyl 4-bromo-1-(3,5-difluorobenzyl)-1H-imidazole-2-carboxylate (6.2 g, 77% yield) as a white solid.

LCMS (ESI) [M+H]⁺: 332.12.

### Step 2: (4-bromo-1-(3,5-difluorobenzyl)-1H-imidazol-2-yl)methanol

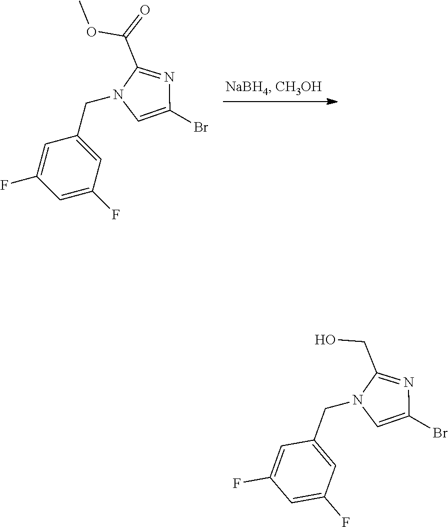 [SMILES: COC(=O)c1nc(Br)cn1Cc1cc(F)cc(F)c1 ; OCc1nc(Br)cn1Cc1cc(F)cc(F)c1]

A mixture of methyl 4-bromo-1-(3,5-difluorobenzyl)-1H-imidazole-2-carboxylate (6 g, 18.2 mmol) and sodium borohydride (2.2 g, 54 mmol) in methanol (20 mL) was stirred for 5 hours at room temperature. The resulting mixture was concentrated under reduced pressure. The residue was purified by silica gel column chromatography, eluted with PE/EA (7:3) to afford (4-bromo-1-(3,5-difluorobenzyl)-1H-imidazol-2-yl)methanol (4.7 g, 85% yield) as a white solid

LCMS (ESI) [M+H]⁺: 303.11

### Step 3: 4-bromo-1-(3,5-difluorobenzyl)-2-(fluoromethyl)-1H-imidazole

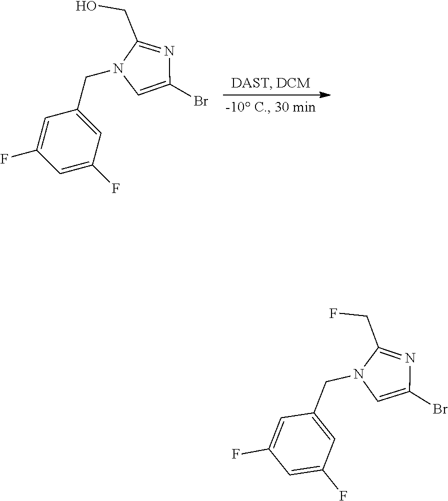 [SMILES: OCc1nc(Br)cn1Cc1cc(F)cc(F)c1 ; FCc1nc(Br)cn1Cc1cc(F)cc(F)c1]

A mixture of methyl (4-bromo-1-(3,5-difluorobenzyl)-1H-imidazol-2-yl)methanol (2 g, 6.6 mmol) and DAST (1.2 g, 7.3 mmol) in DCM (20 mL) was stirred for 5 hours at −10° C. The resulting mixture was concentrated under reduced pressure. The residue was purified by silica gel column chromatography, eluted with PE/EA (7:3) to afford 4-bromo-1-(3,5-difluorobenzyl)-2-(fluoromethyl)-1H-imidazole (630 mg, 31% yield) as a white solid

LCMS (ESI) [M+H]⁺: 306.10

### Step 4: N-(1-(3,5-difluorobenzyl)-2-(fluoromethyl)-1H-imidazol-4-yl)-2-hydroxypropanamide

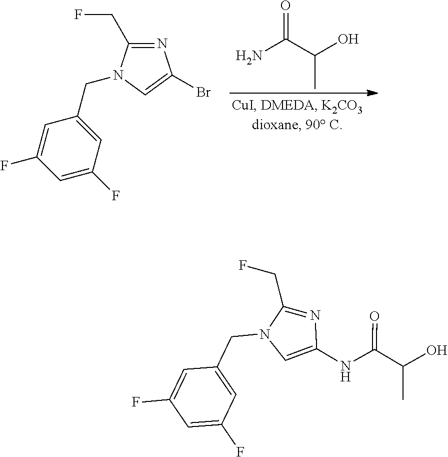

A mixture of 4-bromo-1-(3,5-difluorobenzyl)-2-(fluoromethyl)-1H-imidazole (400 mg, 1.3 mmol), lactamide (117 mg, 1.3 mmol), copper iodide (24.8 mg, 0.13 mmol), K₂CO₃ (361 mg, 2.62 mmol) and DMEDA (23 mg, 0.262 mmol) in dioxane (5 mL) was stirred for 2 hours at 90° C. under a nitrogen atmosphere. The resulting mixture was concentrated under vacuum. The crude residue was loaded onto a silica gel column and eluted with DCM/MeOH (98:2). This yielded N-(1-(3,5-difluorobenzyl)-2-(fluoromethyl)-1H-imidazol-4-yl)-2-hydroxypropanamide (170 mg, 41% yield) as a yellow oil. LCMS (ESI) [M+H]⁺: 314.28.

### Step 5: 1-((1-(3,5-difluorobenzyl)-2-(fluoromethyl)-1H-imidazol-4-yl)amino)-1-oxopropan-2-yl 4-methylbenzenesulfonate

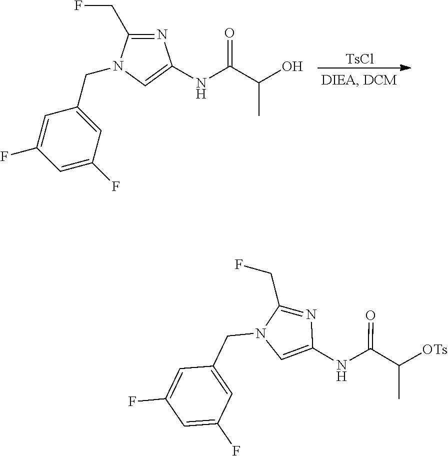 [SMILES: CC(O)C(=O)Nc1cn(Cc2cc(F)cc(F)c2)c(CF)n1 ; "TsCl" ; Cc1ccc(S(=O)(=O)OC(C)C(=O)Nc2cn(Cc3cc(F)cc(F)c3)c(CF)n2)cc1]

To a mixture of N-(1-(3,5-difluorobenzyl)-2-(fluoromethyl)-1H-imidazol-4-yl)-2-hydroxypropanamide (170 mg, 0.54 mmol) and DIEA (136 mg, 1.1 mmol) in DCM (2 mL) was added tosyl chloride (103.45 mg, 0.54 mmol) in portions at 0° C. under a nitrogen atmosphere. The resulting mixture was stirred at 25° C. for 24 hours. Upon completion of the reaction, the mixture was concentrated under reduced pressure and the crude residue was purified by silica gel column chromatography, eluted with DCM/MeOH (10:1) to afford 1-((1-(3,5-difluorobenzyl)-2-(fluoromethyl)-1H-imidazol-4-yl)amino)-1-oxopropan-2-yl 4-methylbenzenesulfonate (138 mg, 54.4% yield) as a yellow oil. LCMS (ESI) [M+H]⁺:468.46.

### Step 6: 4-((3S)-1-(1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide

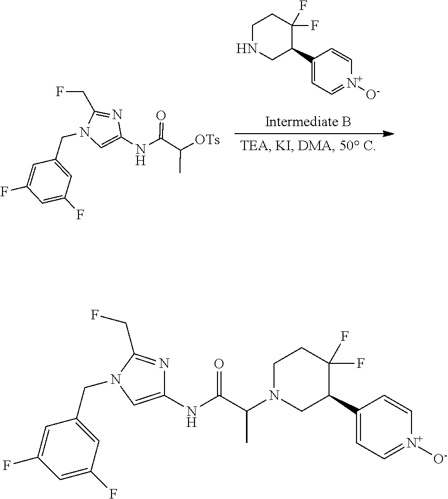 [SMILES: Cc1ccc(S(=O)(=O)OC(C)C(=O)Nc2cn(Cc3cc(F)cc(F)c3)c(CF)n2)cc1 ; [O-][n+]1ccc([C@H]2CNCCC2(F)F)cc1 ; CC(C(=O)Nc1cn(Cc2cc(F)cc(F)c2)c(CF)n1)N1CCC(F)(F)[C@@H](c2cc[n+]([O-])cc2)C1]

Into a 8-mL sample vial was placed 1-((1-(3,5-difluorobenzyl)-2-(fluoromethyl)-1H-imidazol-4-yl)amino)-1-oxopropan-2-yl 4-methylbenzenesulfonate (138 mg, 0.30 mmol), Intermediate B (63.3 mg, 0.30 mmol), TEA (96 uL), KI (cat) and DMA (2 mL). The mixture was stirred at 50° C. for 5 hours until LCMS suggested the completion of the reaction by complete consumption of the starting material. The resulting solution was allowed to cool down to room temperature, poured into water (10 mL) and extracted with EA (3×10 mL). Organic layers were combined, dried over MgSO₄, filtered and concentrated. The crude residue was purified by silica gel chromatography (methanol:dichloromethane=1:15) to give 4-((3S)-1-(1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide (27 mg, 18%) as a light yellow solid. LCMS (ESI) [M+H]⁺: 510.19.

### Step 7: 4-((S)-1-((S)-1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide and 4-((S)-1-((R)-1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide

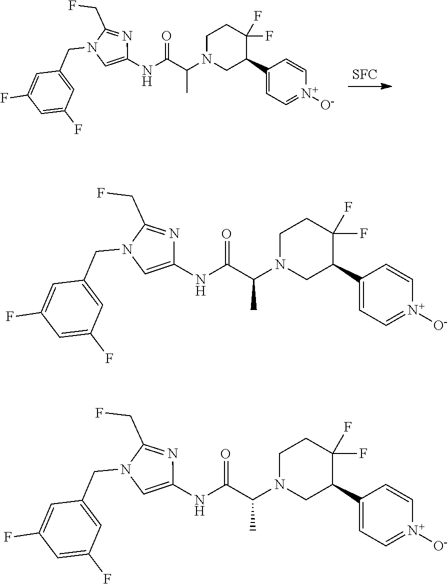

4-((3S)-1-(1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide (27.0 mg) was separated by prep-Chiral-HPLC Column: CHIRALPAK IF, 2×25 cm, 5 μm; Mobile Phase A: Hex (10 mM NH₃-MeOH), Mobile Phase B: EtOH; Flow rate: 40 mL/min; Gradient: isocratic 50% B; Wave Length: 212/268 nm; RT1 (min): 13.6; RT2 (min): 17.7; Sample Solvent: ETOH:DCM=1:1; Injection Volume: 0.8 mL; Number Of Runs: 5 to afford 4-((S)-1-((S)-1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide (Compound 60) (Method 0, 2.06 min, peak 1, 4.6 mg, 100% e.e.) and 4-((S)-1-((R)-1-((1-(3,5-difluorobenzyl)-2-(fluoromethyl)-1H-imidazol-4-yl)amino)-1-oxopropan-2-yl)-4,4-difluoropiperidin-3-yl)pyridine 1-oxide (Compound 61) (Method 0, 2.97 min, peak 2, 3.2 mg, 100% e.e.).

TABLE 18

| Compound# | Characterization Data |
|---|---|
| 60 | LCMS (ESI) [M + H]⁺: 510.2. |
| | ¹H NMR (400 MHz, Methanol-d₄) δ 8.28 (d, J = 7.0 Hz, 2H), 7.57 (d, J = 6.5 Hz, 2H), |
| | 7.40 (d, J = 2.9 Hz, 1H), 6.97 − 6.78 (m, 3H), 5.41 (d, J = 48.8 Hz, 2H), 5.31 (s, 2H), |
| | 3.71 − 3.56 (m, 1H), 3.51 (q, J = 7.1 Hz, 1H), 3.09 − 2.76 (m, 4H), 2.42 − 2.14 (m, 2H), |
| | 1.32 (d, J = 7.0 Hz, 3H). |
| 61 | LCMS (ESI) [M + H]⁺: 510.2. |
| | ¹H NMR (400 MHz, Methanol-d₄) δ 8.29 (d, J = 7.0 Hz, 2H), 7.58 (d, J = 6.6 Hz, 2H), |
| | 7.40 (d, J = 2.9 Hz, 1H), 7.04 − 6.75 (m, 3H), 5.42 (d, J = 48.8 Hz, 2H), 5.32 (s, 2H), |
| | 3.70 − 3.45 (m, 2H), 3.10 − 2.88 (m, 3H), 2.70 − 2.59 (m, 1H), 2.37 − 2.15 (m, 2H), |
| | 1.33 (d, J = 7.0 Hz, 3H). |

### Example 41: 4-((S)-4,4-difluoro-1-((S)-1-((1-(5-fluoro-2-(trifluoromethyl)benzyl)-1H-imidazol-4-yl)amino)-1-oxopropan-2-yl)piperidin-3-yl)pyridine 1-oxide

### Step 1: 4-((S)-4,4-difluoro-1-((S)-1-((1-(5-fluoro-2-(trifluoromethyl)benzyl)-1H-imidazol-4-yl)amino)-1-oxopropan-2-yl)piperidin-3-yl)pyridine 1-oxide

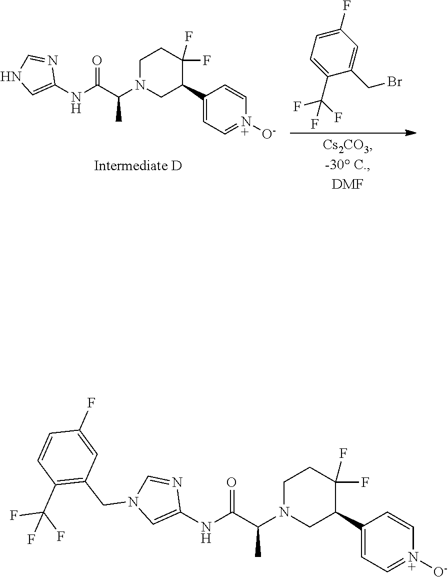 [SMILES: C[C@@H](C(=O)Nc1c[nH]cn1)N1CCC(F)(F)[C@@H](c2cc[n+]([O-])cc2)C1 ; Fc1ccc(C(F)(F)F)c(CBr)c1 ; C[C@@H](C(=O)Nc1cn(Cc2cc(F)ccc2C(F)(F)F)cn1)N1CCC(F)(F)[C@@H](c2cc[n+]([O-])cc2)C1]

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol) and Cs₂CO₃ (43 mg, 0.13 mmol) in DMF (0.5 mL) was added 2-(bromomethyl)-4-fluoro-1-(trifluoromethyl)benzene (24 mg, 0.09 mmol) at −30° C. under a nitrogen atmosphere. The resulting mixture was stirred for 2 hours at −30° C. under a nitrogen atmosphere. The resulting mixture was filtered and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 14% B to 34% B in 9 min; Wave Length: 254 nm/220 nm; RT1 (min): 9.43) to afford 4-((S)-4,4-difluoro-1-((S)-1-((1-(5-fluoro-2-(trifluoromethyl)benzyl)-1H-imidazol-4-yl)amino)-1-oxopropan-2-yl)piperidin-3-yl)pyridine 1-oxide (Compound 62) (19.8 mg, 44% yield) as a white solid.

LCMS [M+H]⁺: 528.1

¹H NMR (400 MHz, DMSO-d₆) δ 10.25 (s, 1H), 8.18 (d, J=6.8 Hz, 2H), 7.89 (dd, J=8.8, 5.4 Hz, 1H), 7.63 (d, J=1.5 Hz, 1H), 7.47-7.34 (m, 3H), 7.25 (d, J=1.5 Hz, 1H), 7.07-6.86 (m, 1H), 5.37 (s, 2H), 3.62-3.53 (m, 1H), 3.51-3.37 (m, 2H), 3.02-2.81 (m, 3H), 2.15-2.01 (m, 2H), 1.17 (d, J=6.9 Hz, 3H).

### Example 42: 4-((S)-1-((S)-1-((1-((3-chloropyridin-4-yl)methyl)-1H-imidazol-4-yl)amino)-1-oxopropan-2-yl)-4,4-difluoropiperidin-3-yl)pyridine 1-oxide

### Step 1: 4-((S)-1-((S)-1-((1-((3-chloropyridin-4-yl)methyl)-1H-imidazol-4-yl)amino)-1-oxopropan-2-yl)-4,4-difluoropiperidin-3-yl)pyridine 1-oxide

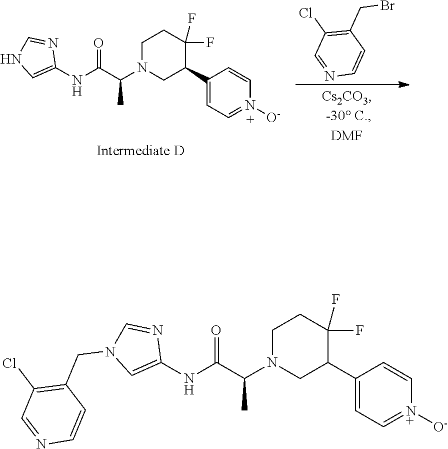

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol) and Cs₂CO₃ (42 mg, 0.13 mmol) in DMF (0.5 mL) was added 4-(bromomethyl)-3-chloropyridine (19 mg, 0.09 mmol) at −30° C. under a nitrogen atmosphere. The resulting mixture was stirred for 2 hours at −30° C. under a nitrogen atmosphere. The resulting mixture was filtered and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.64) to afford 4-((S)-1-((S)-1-((1-((3-chloropyridin-4-yl)methyl)-1H-imidazol-4-yl)amino)-1-oxopropan-2-yl)-4,4-difluoropiperidin-3-yl)pyridine 1-oxide (Compound 63) (16.7 mg, 40% yield) as a yellow solid.

LCMS [M+H]⁺: 477.1

¹H NMR (400 MHz, DMSO-d₆) δ 10.26 (s, 1H), 8.66 (s, 1H), 8.52 (d, J=4.9 Hz, 1H), 8.24-8.13 (m, 2H), 7.64 (d, J=1.6 Hz, 1H), 7.41-7.29 (m, 3H), 6.98 (d, J=5.0 Hz, 1H), 5.35 (s, 2H), 3.65-3.48 (m, 1H), 3.05-2.83 (m, 3H), 2.55 (d, J=4.7 Hz, 1H), 2.21-1.97 (m, 2H), 1.18 (d, J=6.8 Hz, 3H).

### Example 43: 4-((S)-1-((S)-1-((1-(3-chloro-2-fluorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3-chloro-2-fluorobenzyl)-1H-imidazol-4-yl)amino)-1-oxopropan-2-yl)-4,4-difluoropiperidin-3-yl)pyridine 1-oxide

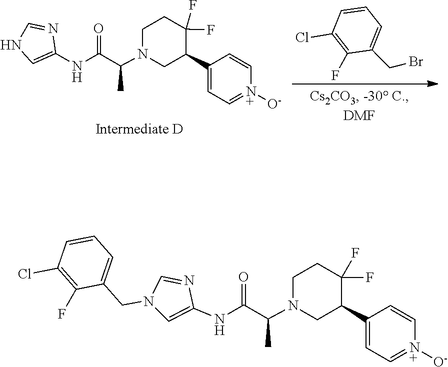 [SMILES: C[C@@H](C(=O)Nc1c[nH]cn1)N1CCC(F)(F)[C@@H](c2cc[n+]([O-])cc2)C1 ; Fc1c(Cl)cccc1CBr ; C[C@@H](C(=O)Nc1cn(Cc2cccc(Cl)c2F)cn1)N1CCC(F)(F)[C@@H](c2cc[n+]([O-])cc2)C1]

To a mixture of 4-[(3S)-4,4-difluoro-1-[(15)-1-[(1H-imidazol-4-yl)carbamoyl]ethyl]piperidin-3-yl]pyridin-1-ium-1-olate (Intermediate D) (30 mg, 0.09 mmol) and Cs₂CO₃ (42 mg, 0.13 mmol) in DMF (0.5 mL) was added 1-(bromomethyl)-3-chloro-2-fluorobenzene (25 mg, 0.11 mmol) at −30° C. under a nitrogen atmosphere. The resulting mixture was stirred for 2 hours at −30° C. under a nitrogen atmosphere. The resulting mixture was filtered and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.95) to afford 4-((S)-1-((S)-1-((1-(3-chloro-2-fluorobenzyl)-1H-imidazol-4-yl)amino)-1-oxopropan-2-yl)-4,4-difluoropiperidin-3-yl)pyridine 1-oxide (Compound 64) (13.7 mg, 32% yield) as a light yellow solid.

LCMS [M+H]⁺: 494.1

¹H NMR (400 MHz, DMSO-d₆) δ 10.60 (s, 1H), 8.15 (d, J=6.6 Hz, 2H), 7.80 (s, 1H), 7.58-7.49 (m, 1H), 7.33 (d, J=6.6 Hz, 2H), 7.25 (d, J=4.0 Hz, 2H), 7.22-7.14 (m, 1H), 5.26 (s, 2H), 3.77-3.48 (m, 3H), 3.22-3.05 (m, 2H), 2.79-2.72 (m, 1H), 2.30-2.13 (m, 2H), 1.27 (d, J=6.7 Hz, 3H).

### Example 44: 4-((S)-4,4-difluoro-1-((S)-1-oxo-1-((1-(2,4,5-trifluorobenzyl)-1H-imidazol-4-yl)amino)propan-2-yl)piperidin-3-yl)pyridine 1-oxide

### Step 1: 4-((S)-4,4-difluoro-1-((S)-1-oxo-1-((1-(2,4,5-trifluorobenzyl)-1H-imidazol-4-yl)amino)propan-2-yl)piperidin-3-yl)pyridine 1-oxide

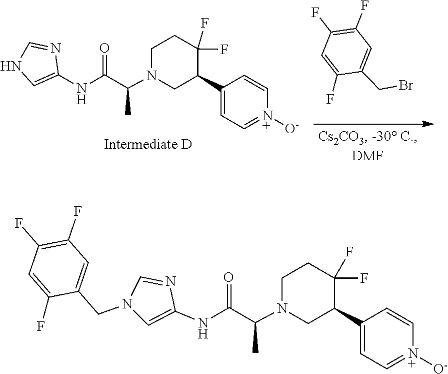 [SMILES: C[C@@H](C(=O)Nc1c[nH]cn1)N1CCC(F)(F)[C@@H](c2cc[n+]([O-])cc2)C1 ; Fc1cc(F)c(CBr)cc1F ; C[C@@H](C(=O)Nc1cn(Cc2cc(F)c(F)cc2F)cn1)N1CCC(F)(F)[C@@H](c2cc[n+]([O-])cc2)C1]

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.09 mmol) and Cs₂CO₃ (42 mg, 0.13 mmol) in DMF (0.5 mL) was added 1-(bromomethyl)-2,4,5-trifluorobenzene (25 mg, 0.11 mmol) at −30° C. under a nitrogen atmosphere. The resulting mixture was stirred for 2 hours at −30° C. under a nitrogen atmosphere. The resulting mixture was filtered and the filter cake was washed with DMF (0.5 mL). The filtrate was concentrated under reduced pressure. The crude product was purified by Prep-HPLC with the following conditions (Column: Kinetex EVO C18 Column, 30×150, Sum; Mobile Phase A: Water (0.1% FA), Mobile Phase B: ACN; Flow rate: 60 mL/min; Gradient: 5% B to 5% B in 1 min, 5% B to 15% B in 2 min, 15% to 35% B in 10 min; Wave Length: 254 nm/220 nm; RT1 (min): 8.9) to afford 4-((S)-4,4-difluoro-1-((S)-1-oxo-1-((1-(2,4,5-trifluorobenzyl)-1H-imidazol-4-yl)amino)propan-2-yl)piperidin-3-yl)pyridine 1-oxide (Compound 65) (11 mg, 26% yield) as a white solid.

LCMS (ESI) [M+H]⁺: 496.1

¹H NMR (400 MHz, Methanol-d₄) δ 8.38-8.23 (m, 2H), 7.67-7.53 (m, 3H), 7.43-7.19 (m, 3H), 5.21 (s, 2H), 3.62-3.47 (m, 2H), 3.05-2.94 (m, 3H), 2.66-2.61 (m, 1H), 2.21-2.11 (m, 2H), 1.32 (d, J=7.0 Hz, 3H).

### Example 45: 4-((S)-1-((S)-1-((1-(2,3-dichlorobenzyl)-1H-imidazol-4-yl)amino)-1-oxopropan-2-yl)-4,4-difluoropiperidin-3-yl)pyridine 1-oxide

### Step 1: 4-((S)-1-((S)-1-((1-(2,3-dichlorobenzyl)-1H-imidazol-4-yl)amino)-1-oxopropan-2-yl)-4,4-difluoropiperidin-3-yl)pyridine 1-oxide

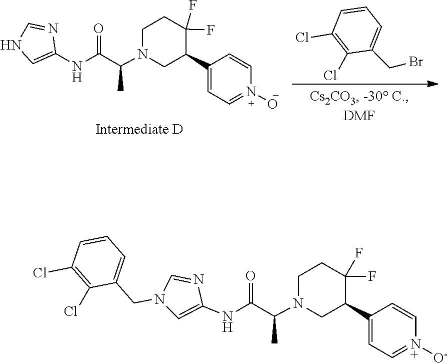

To a mixture of 4-[(3S)-4,4-difluoro-1-[(1S)-1-[(1H-imidazol-4-yl)carbamoyl]ethyl]piperidin-3-yl]pyridin-1-ium-1-olate (Intermediate D) (30 mg, 0.08 mmol) and Cs₂CO₃ (42 mg, 0.13 mmol) in DMF (0.5 mL) were added 1-(bromomethyl)-2,3-dichlorobenzene (22 mg, 0.09 mmol) at −30° C. under a nitrogen atmosphere. The resulting mixture was stirred for 2 hours at −30° C. under a nitrogen atmosphere. The resulting mixture was filtered and the filter cake was washed with DMF (0.5 mL). The filtrate was concentrated under reduced pressure. The crude product was purified by Prep-HPLC with the following conditions (Column: XSelect CSH Fluoro Phenyl 30×150 mm, 5 μm; Mobile Phase A: Water (0.1% FA), Mobile Phase B: ACN; Flow rate: 60 mL/min; Gradient: 23% B to 43% B in 9 min; Wave Length: 254 nm/220 nm; RT1 (min): 8.78) to afford 4-((S)-1-((S)-1-((1-(2,3-dichlorobenzyl)-1H-imidazol-4-yl)amino)-1-oxopropan-2-yl)-4,4-difluoropiperidin-3-yl)pyridine 1-oxide (Compound 66) (17.6 mg, 39% yield) as a light yellow solid.

LCMS [M+H]⁺: 510.2

¹H NMR (400 MHz, DMSO-d₆) δ 10.23 (s, 1H), 8.19-8.12 (m, 2H), 7.70-7.60 (m, 2H), 7.45-7.34 (m, 3H), 7.25 (d, J=1.5 Hz, 1H), 7.18-7.13 (m, 1H), 5.32 (s, 2H), 3.62-3.53 (m, 1H), 3.51-3.41 (m, 1H), 3.01-2.82 (m, 3H), 2.53-2.48 (m, 1H), 2.23-2.03 (m, 2H), 1.17 (d, J=6.9 Hz, 3H).

### Example 46: 2-(4,4-difluoro-3-(1-methyl-6-oxo-1,6-dihydropyridin-3-yl)piperidin-1-yl)-N-(1-(3,5-difluorobenzyl)-1H-imidazol-4-yl)propanamide

### Step 1: tert-butyl 3-(1-methyl-6-oxo-1,6-dihydropyridin-3-yl)-4-oxopiperidine-1-carboxylate

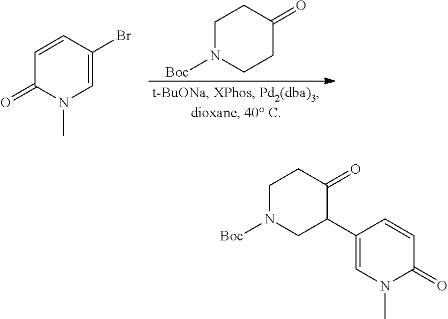

A solution of 5-bromo-1-methylpyridin-2-one (2.0 g, 10.64 mmol), tert-butyl 4-oxopiperidine-1-carboxylate (2.5 g, 12.76 mmol), XPhos (0.5 g, 1.06 mmol), Pd₂(dba)₃ (2.0 g, 2.13 mmol) and t-BuONa (3.1 g, 31.91 mmol) in dioxane (25 mL) was stirred for overnight at 40° C. under a nitrogen atmosphere. The mixture was allowed to cool down to room temperature. The reaction was quenched with water (30 mL) at 0° C. The aqueous layer was extracted with EtOAc (3×30 mL). The combined organic layers were dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% NH₄HCO₃), 0% to 100% gradient in 20 min; detector, UV 254 nm. This resulted in tert-butyl 3-(1-methyl-6-oxo-1,6-dihydropyridin-3-yl)-4-oxopiperidine-1-carboxylate (850 mg, 26% yield) as a light-yellow oil. LCMS (ESI) [M+H]⁺: 307.

### Step 2: tert-butyl 4,4-difluoro-3-(1-methyl-6-oxo-1,6-dihydropyridin-3-yl)piperidine-1-carboxylate

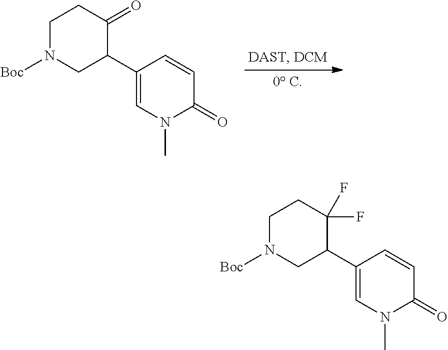 [SMILES: Cn1cc(C2CN(C(=O)OC(C)(C)C)CCC2=O)ccc1=O ; Cn1cc(C2CN(C(=O)OC(C)(C)C)CCC2(F)F)ccc1=O]

To a mixture of tert-butyl 3-(1-methyl-6-oxo-1,6-dihydropyridin-3-yl)-4-oxopiperidine-1-carboxylate (840 mg, 2.74 mmol) in DCM (10 mL) was added DAST (1.3 g, 8.23 mmol) dropwise at 0° C. The resulting mixture was stirred overnight at 0° C. under a nitrogen atmosphere. The reaction was quenched by the addition of sat. aq. NaHCO₃ (15 mL) at 0° C. The aqueous layer was extracted with CH₂Cl₂ (3×20 mL). The combined organic layers were dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% NH₄HCO₃), 0% to 100% gradient in 20 min; detector, UV 220 nm. This resulted in tert-butyl 4,4-difluoro-3-(1-methyl-6-oxo-1,6-dihydropyridin-3-yl)piperidine-1-carboxylate (350 mg, 39% yield) as a brown solid. LCMS (ESI) [M+H]⁺: 329.

### Step 3: 5-(4,4-difluoropiperidin-3-yl)-1-methylpyridin-2(1H)-one

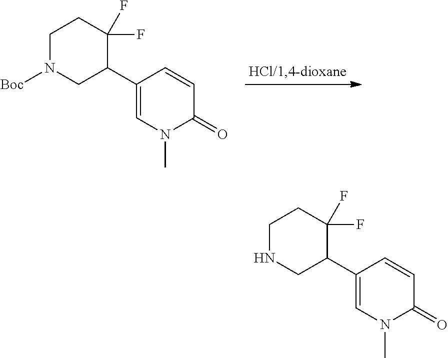 [SMILES: Cn1cc(C2CN(C(=O)OC(C)(C)C)CCC2(F)F)ccc1=O ; Cn1cc(C2CNCCC2(F)F)ccc1=O]

A solution of tert-butyl 4,4-difluoro-3-(1-methyl-6-oxo-1,6-dihydropyridin-3-yl)piperidine-1-carboxylate (340 mg, 1.04 mmol) in 4M HCl in 1,4-dioxane (10 mL) was stirred for 1 hour at room temperature under an ambient atmosphere. The resulting mixture was concentrated under vacuum. The residue was purified by reversed-phase flash chromatography with the following conditions: column, C18 silica gel; mobile phase, MeCN in Water (0.11% NH₄HCO₃), 0% to 100% gradient in 20 min; detector, UV 220 nm. This resulted 5-(4,4-difluoropiperidin-3-yl)-1-methylpyridin-2(1H)-one (185 mg, 78% yield) as a white solid. LCMS (ESI) [M+H]⁺: 229.

### Step 4: (S)-5-(4,4-difluoropiperidin-3-yl)-1-methylpyridin-2(1H)-one and (R)-5-(4,4-difluoropiperidin-3-yl)-1-methylpyridin-2(1H)-one

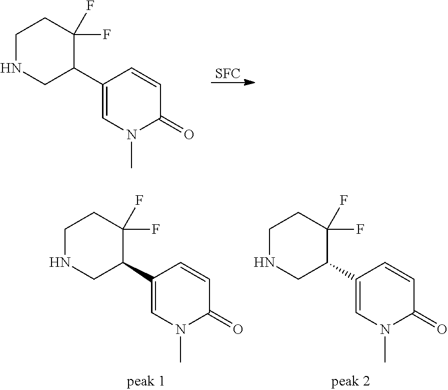

5-(4,4-difluoropiperidin-3-yl)-1-methylpyridin-2-one (180 mg) was purified by Prep-Chiral-HPLC with the following conditions (Column: CHIRALCEL AY-H, 2×25 cm, 5 μm; Mobile Phase A: Hex (10 mM NH₃-MeOH), Mobile Phase B: EtOH; Flow rate: 40 mL/min; Gradient: isocratic 30% B; Wave Length: 225/235 nm; RT1 (min): 7.2; RT2 (min): 11.4) to afford (S)-5-(4,4-difluoropiperidin-3-yl)-1-methylpyridin-2(1H)-one (73 mg, peak 1, 100% e.e.) and (R)-5-(4,4-difluoropiperidin-3-yl)-1-methylpyridin-2(1H)-one (70 mg, peak 2, 100% e.e.).

LCMS (ESI) [M+H]⁺: 229.

### Step 5: 2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide

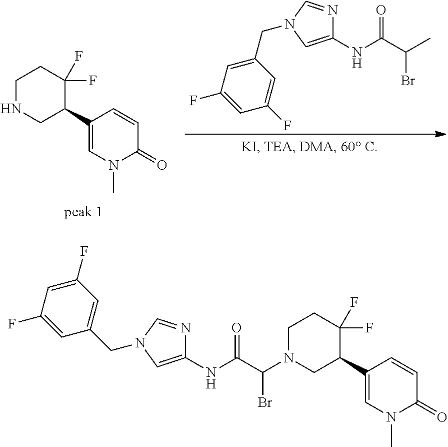 [SMILES: Cn1cc([C@H]2CNCCC2(F)F)ccc1=O ; CC(Br)C(=O)Nc1cn(Cc2cc(F)cc(F)c2)cn1 ; Cn1cc([C@H]2CN(C(Br)C(=O)Nc3cn(Cc4cc(F)cc(F)c4)cn3)CCC2(F)F)ccc1=O]

A solution of (S)-5-(4,4-difluoropiperidin-3-yl)-1-methylpyridin-2(1H)-one (73 mg, 0.32 mmol), 2-bromo-N-(1-(3,5-difluorobenzyl)-1H-imidazol-4-yl)propanamide (121 mg, 0.35 mmol), KI (58 mg, 0.35 mmol) and TEA (162 mg, 1.6 mmol) in DMA (5 mL) was stirred for overnight at 60° C. under a nitrogen atmosphere. The mixture was allowed to cool down to room temperature. The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (78 mg, 50% yield) as a white solid. LCMS (ESI) [M+H]⁺: 492.

### Step 6: (S)-2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide and (R)-2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide

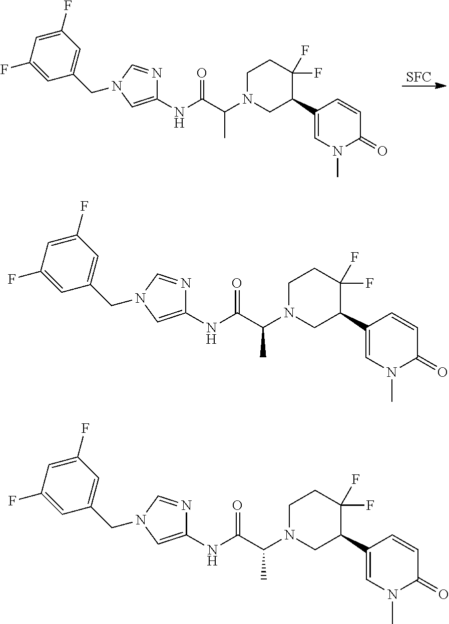 [SMILES: CC(C(=O)Nc1cn(Cc2cc(F)cc(F)c2)cn1)N1CCC(F)(F)[C@@H](c2ccc(=O)n(C)c2)C1 ; "SFC" ; C[C@@H](C(=O)Nc1cn(Cc2cc(F)cc(F)c2)cn1)N1CCC(F)(F)[C@@H](c2ccc(=O)n(C)c2)C1 ; C[C@H](C(=O)Nc1cn(Cc2cc(F)cc(F)c2)cn1)N1CCC(F)(F)[C@@H](c2ccc(=O)n(C)c2)C1]

2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (78 mg) was purified by Prep-Chiral-HPLC with the following conditions (Column: CHIRALPAK ID, 2×25 cm, 5 μm; Mobile Phase A: MtBE (10 mM NH₃-MeOH), Mobile Phase B: MEOH; Flow rate: 20 mL/min; Gradient: isocratic 30% B; Wave Length: 234/220 nm; RT1 (min): 3.99; RT2 (min): 7.47) to afford (S)-2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (Compound 67) (Method P, 0.57 min, peak 1, 25.8 mg, 100% e.e.) and (R)-2-((S)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (Compound 68) (Method P, 1.09 min, peak 2, 29.3 mg, 100% e.e.).

TABLE 19

| Compound# | Characterization Data |
|---|---|
| 67 | LCMS (ESI) [M + H]⁺: 492.2 |
| | ¹H NMR (400 MHz, DMSO-d₆) δ 10.23 (s, 1H), 7.81 − 7.61 (m, 2H), 7.37 − 7.17 (m, 3H), 7.14 − |
| | 6.93 (m, 2H), 6.33 (d, J = 9.3 Hz, 1H), 5.17 (s, 2H), 3.56 − 3.50 (m, 1H), 3.46 − 3.40 (m, 3H), |
| | 3.16 − 2.93 (m, 3H), 2.68 − 2.62 (m, 1H), 2.25 − 2.03 (m, 2H), 1.19 (d, J = 6.7 Hz, 3H). |
| 68 | LCMS (ESI) [M + H]⁺: 492.1 |
| | ¹H NMR (400 MHz, DMSO-d₆) δ 10.19 (s, 1H), 7.68 − 7.64 (m, 2H), 7.44 − 7.35 (m, 1H), 7.30 |
| | (d, J = 1.6 Hz, 1H), 7.27 − 7.15 (m, 1H), 7.09 − 7.01 (m, 2H), 6.34 (d, J = 9.4 Hz, 1H), 5.17 (s, |
| | 2H), 3.56 − 3.51 (m, 1H), 3.40 (s, 3H), 3.12 − 3.08 (m, 1H), 2.89 − 2.73 (m, 3H), 2.49 − 2.43 (m, |
| | 1H), 2.23 − 2.06 (m, 2H), 1.17 (d, J = 6.9 Hz, 3H). |

### Step 7: 2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide

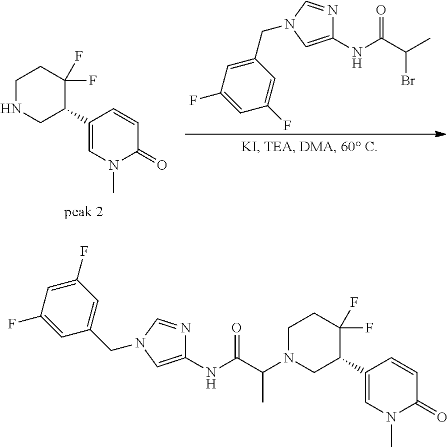 [SMILES: Cn1cc([C@@H]2CNCCC2(F)F)ccc1=O ; CC(Br)C(=O)Nc1cn(Cc2cc(F)cc(F)c2)cn1 ; CC(C(=O)Nc1cn(Cc2cc(F)cc(F)c2)cn1)N1CCC(F)(F)[C@H](c2ccc(=O)n(C)c2)C1]

A solution of (R)-5-(4,4-difluoropiperidin-3-yl)-1-methylpyridin-2(1H)-one (70 mg, 0.31 mmol), 2-bromo-N-(1-(3,5-difluorobenzyl)-1H-imidazol-4-yl)propanamide (116 mg, 0.34 mmol), KI (56 mg, 0.34 mmol) and TEA (155 mg, 1.5 mmol) in DMA (1.5 mL) was stirred for overnight at 60° C. under a nitrogen atmosphere. The mixture was allowed to cool down to room temperature. The residue was dissolved in water (3 mL). The resulting mixture was extracted with EtOAc (3×5 mL). The combined organic layers were washed with water (3×6 mL), dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in 2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (70 mg, 46% yield) as a white solid. LCMS (ESI) [M+H]⁺: 492.

### Step 8: (S)-2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide and (R)-2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide

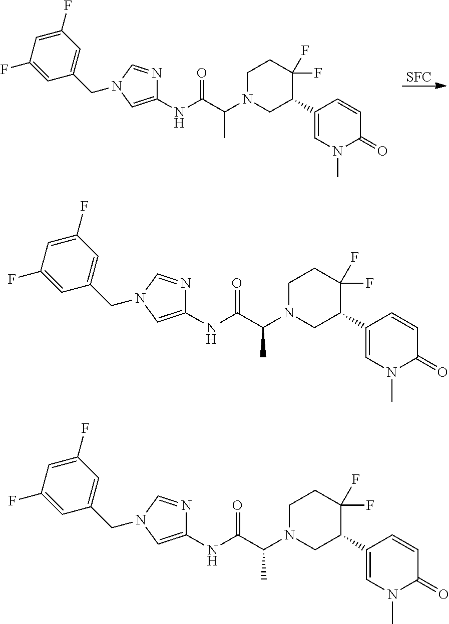 [SMILES: CC(C(=O)Nc1cn(Cc2cc(F)cc(F)c2)cn1)N1CCC(F)(F)[C@H](c2ccc(=O)n(C)c2)C1 ; "SFC" ; C[C@@H](C(=O)Nc1cn(Cc2cc(F)cc(F)c2)cn1)N1CCC(F)(F)[C@H](c2ccc(=O)n(C)c2)C1 ; C[C@H](C(=O)Nc1cn(Cc2cc(F)cc(F)c2)cn1)N1CCC(F)(F)[C@H](c2ccc(=O)n(C)c2)C1]

2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (73 mg) was purified by Prep-Chiral-HPLC with the following conditions (Column: CHIRAL ART Cellulose-SC, 3×25 cm, 5 μm; Mobile Phase A: MtBE (10 mM NH₃-MeOH), Mobile Phase B: MEOH; Flow rate: 40 mL/min; Gradient: isocratic 30% B; Wave Length: 234/220 nm; RT1 (min): 5.38; RT2 (min): 11.59) to afford: (S)-2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (Compound 69) (Method Q, 0.78 min, peak 1, 27.0 mg, >99% e.e.) and (R)-2-((R)-4,4-difluoro-3-(1-methyl-6-oxo-1,6-dihydropyridin-3-yl)piperidin-1-yl)-N-(1-(3,5-difluorobenzyl)-1H-imidazol-4-yl)propanamide (Compound 70) (Method Q, 1.81 min, peak 2, 21.8 mg, >99% e.e.).

TABLE 20

| Compound# | Characterization Data |
|---|---|
| 69 | LCMS (ESI) [M + H]⁺: 492.2 |
| | ¹H NMR (400 MHz, DMSO-d₆) δ 10.19 (s, 1H), 7.69 − 7.62 (m, 2H), 7.39 − 7.32 (s, 1H), 7.30 |
| | (d, J = 1.9 Hz, 1H), 7.26 − 7.17 (m, 1H), 7.10 − 6.95 (m, 2H), 6.33 (d, J = 9.3 Hz, 1H), 5.17 (s, |
| | 2H), 3.60 − 3.43 (m, 1H), 3.40 (s, 3H), 3.19 − 3.06 (m, 1H), 2.92 − 2.83 (m, 2H), 2.69 − 2.56 (m, |
| | 2H), 2.19 − 2.02 (m, 2H), 1.18 (d, J = 6.8 Hz, 3H). |
| 70 | LCMS (ESI) [M + H]⁺: 492.1 |
| | ¹H NMR (400 MHz, DMSO-d₆) δ 10.18 (s, 1H), 7.73 − 7.56 (m, 2H), 7.45 − 7.31 (m, 1H), 7.29 |
| | (d, J = 1.5 Hz, 1H), 7.24 − 7.14 (m, 1H), 7.11 − 7.00 (m, 2H), 6.34 (d, J = 9.4 Hz, 1H), 5.17 (s, |
| | 2H), 3.56 − 3.49 (m, 1H), 3.40 (s, 3H), 3.15 − 3.01 (m, 1H), 2.92 − 2.77 (m, 1H), 2.52 − 2.43 (m, |
| | 1H), 2.23 − 2.02 (m, 2H), 1.16 (d, J = 6.9 Hz, 3H). |

### Example 47: 2-(4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide

### Step 1: (5-bromo-2-methoxypyridin-3-yl)methanol

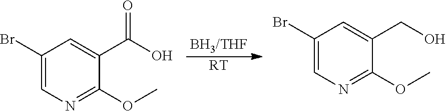

To a mixture of 5-bromo-2-methoxynicotinic acid (5.0 g, 21.54 mmol) in THF (50 mL) was added 1M BH₃-THF in THF (32.3 mL, 32.3 mmol) dropwise at 0° C. The resulting mixture was stirred at room temperature for 1 hour. The reaction was quenched with MeOH (50 mL) at 0° C. The resulting mixture was concentrated under reduced pressure. The residue was purified by silica gel column chromatography, eluted with PE/EA (5:1) to afford (5-bromo-2-methoxypyridin-3-yl)methanol (3.9 g, 83% yield) as a white solid. LCMS (ESI) [M+H]⁺: 218.

### Step 2: 5-bromo-3-(((tert-butyldimethylsilyl)oxy)methyl)-2-methoxypyridine

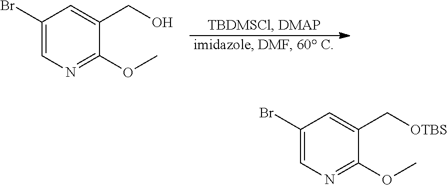

A mixture of (5-bromo-2-methoxypyridin-3-yl)methanol (3.9 g, 17.9 mmol), tert-butylchlorodimethylsilane (13.4 g, 89.4 mmol), imidazole (12.1 g, 178.9 mmol) and DMAP (218 mg, 1.78 mmol) in DMF (40 mL) was stirred at 60° C. for 2 hours. The mixture was allowed to cool down to room temperature. The reaction was quenched with water (80 mL). The resulting mixture was extracted with EtOAc (3×55 mL). The combined organic layers were washed with brine, dried over anhydrous Na₂SO₄. filtered and concentrated in vacuo. The residue was purified by silica gel column chromatography, eluted with PE/EA (10:1) to afford 5-bromo-3-(((tert-butyldimethylsilyl)oxy)methyl)-2-methoxypyridine (5.1 g, 85% yield) as a white solid. LCMS (ESI) [M+H]⁺: 332.

### Step 3: tert-butyl 3-(5-(((tert-butyldimethylsilyl)oxy)methyl)-6-methoxypyridin-3-yl)-4-oxopiperidine-1-carboxylate

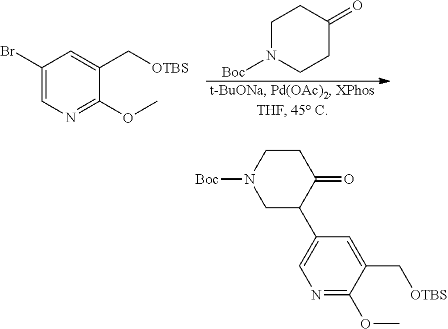

A mixture of 5-bromo-3-(((tert-butyldimethylsilyl)oxy)methyl)-2-methoxypyridine (5.1 g, 15.34 mmol), tert-butyl 4-oxopiperidine-1-carboxylate (3.6 g, 18.41 mmol, 1.2 equiv), t-BuONa (5.9 g, 61.38 mmol), Pd(OAc)₂ (344 mg, 1.53 mmol) and XPhos (1.4 g, 3.06 mmol) in THF (60 mL) was stirred at 45° C. for 2 hours under a nitrogen atmosphere. The mixture was allowed to cool down to room temperature. The resulting mixture was filtered and the filter cake was washed with EtOAc (3×70 mL). The filtrate was concentrated under vacuum. The residue was purified by silica gel column chromatography, eluted with PE/EA (1:1) to afford tert-butyl 3-(5-(((tert-butyldimethylsilyl)oxy)methyl)-6-methoxypyridin-3-yl)-4-oxopiperidine-1-carboxylate (4.2 g, 60% yield) as a yellow oil. LCMS (ESI) [M+H]⁺: 451.

### Step 4: tert-butyl 3-(5-(((tert-butyldimethylsilyl)oxy)methyl)-6-methoxypyridin-3-yl)-4,4-difluoropiperidine-1-carboxylate

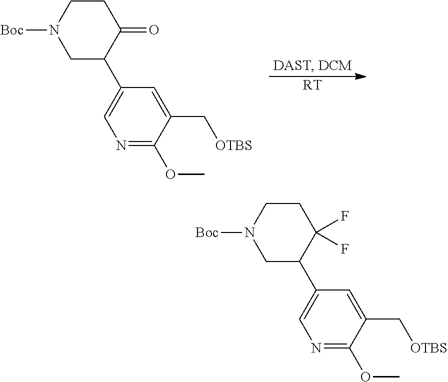

To a mixture of tert-butyl 3-(5-(((tert-butyldimethylsilyl)oxy)methyl)-6-methoxypyridin-3-yl)-4-oxopiperidine-1-carboxylate (4.2 g, 9.3 mmol) in DCM (50 mL) was added DAST (3.0 g, 18.6 mmol) dropwise at 0° C. The resulting mixture was stirred at room temperature overnight. The reaction was quenched with sat. aq. NaHCO₃ (30 mL) at 0° C. The resulting mixture was extracted with CH₂Cl₂ (3×60 mL). The combined organic layers were dried over anhydrous Na₂SO₄ and filtered. The filtrate was concentrated under vacuum. The residue was purified by silica gel column chromatography, eluted with PE/EA (1:1) to afford tert-butyl 3-(5-(((tert-butyldimethylsilyl)oxy)methyl)-6-methoxypyridin-3-yl)-4,4-difluoropiperidine-1-carboxylate (1.2 g, 27% yield) as a yellow oil. LCMS (ESI) [M+H]⁺: 473.

### Step 5: tert-butyl 4,4-difluoro-3-(5-(hydroxymethyl)-6-methoxypyridin-3-yl)piperidine-1-carboxylate

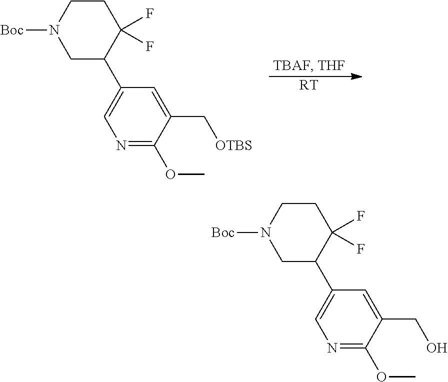

A solution of tert-butyl 3-(5-(((tert-butyldimethylsilyl)oxy)methyl)-6-methoxypyridin-3-yl)-4,4-difluoropiperidine-1-carboxylate (1.2 g, 2.5 mmol) and TBAF 1M in THF (12.7 mL, 12.7 mmol) in THF (20 mL) was stirred at room temperature for 1 hour. The reaction was quenched with water (30 mL). The resulting mixture was extracted with EtOAc (3×45 mL). The combined organic layers were washed with brine, dried over anhydrous Na₂SO₄, filtered and concentrated. The crude residue was purified by Prep-HPLC with the following conditions (Column: YMC-Actus Triart C18 ExRS 30×150 mm, 5 μm; Mobile Phase A: Water (10 mmol/L NH₄HCO_{3+0.05}% NH₃·H₂O), Mobile Phase B: ACN; Flow rate: 60 mL/min; Gradient: 10% B to 10% B in 1.5 min, 10% B to 10% B in 2 min, 34% B to 51% B in 10 min; Wave Length: 254 nm/220 nm; RT1 (min): 10.12) to afford tert-butyl 4,4-difluoro-3-(5-(hydroxymethyl)-6-methoxypyridin-3-yl)piperidine-1-carboxylate (510 mg, 56% yield) as an off-white solid. LCMS (ESI) [M+H]⁺: 359.

### Step 6: 5-(4,4-difluoropiperidin-3-yl)-3-(hydroxymethyl)pyridin-2(1H)-one

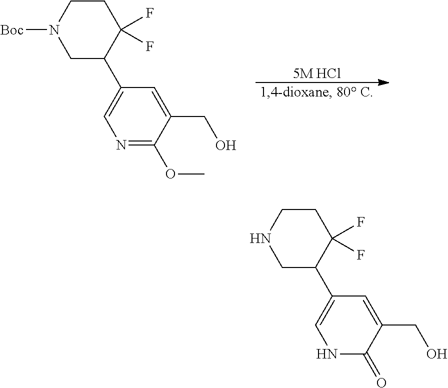 [SMILES: COc1ncc(C2CN(C(=O)OC(C)(C)C)CCC2(F)F)cc1CO ; "1,4-dioxane, 80° C." ; "5M HCl" ; O=c1[nH]cc(C2CNCCC2(F)F)cc1CO]

To a mixture of tert-butyl 4,4-difluoro-3-(5-(hydroxymethyl)-6-methoxypyridin-3-yl)piperidine-1-carboxylate (510 mg, 1.4 mmol) in 1,4-dioxane (20 mL) was added 5M HCl in water (14 mL, 71.2 mmol) dropwise at room temperature. The resulting mixture was stirred at 80° C. for 2 hours. The mixture was allowed to cool down to room temperature. The mixture was adjusted to pH 7 with 1M aq. NaOH. The resulting mixture was concentrated under reduced pressure. The residue was dissolved in MeOH (10 mL). The resulting mixture was filtered and the precipitate was washed with MeOH (3×5 mL). The resulting mixture was concentrated under vacuum. The residue was purified by reversed-phase flash chromatography with the following conditions: column, C18 silica gel; mobile phase, MeCN in Water (10 mmol/L NH₄HCO₃), 5% to 100% gradient in 30 min; detector, UV 254 nm and UV 220 nm. This resulted in 5-(4,4-difluoropiperidin-3-yl)-3-(hydroxymethyl)pyridin-2(1H)-one (160 mg, 46% yield) as a yellow oil. LCMS (ESI) [M+H]⁺: 245.

### Step 7: 2-(4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide

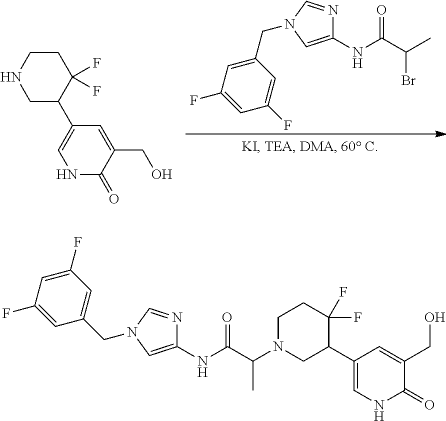 [SMILES: O=c1[nH]cc(C2CNCCC2(F)F)cc1CO ; CC(Br)C(=O)Nc1cn(Cc2cc(F)cc(F)c2)cn1 ; CC(C(=O)Nc1cn(Cc2cc(F)cc(F)c2)cn1)N1CCC(F)(F)C(c2c[nH]c(=O)c(CO)c2)C1]

A solution of 5-(4,4-difluoropiperidin-3-yl)-3-(hydroxymethyl)pyridin-2(1H)-one (160 mg, 0.65 mmol), 2-bromo-N-(1-(3,5-difluorobenzyl)-1H-imidazol-4-yl)propanamide (270 mg, 0.78 mmol), KI (130 mg, 0.78 mmol) and TEA (331 mg, 3.3 mmol) in DMA (5 mL) was stirred at 60° C. for 1 hour. The mixture was allowed to cool down to room temperature. The reaction was quenched with water (7 mL). The resulting mixture was extracted with EtOAc (3×10 mL). The combined organic layers were washed with brine, dried over anhydrous Na₂SO₄, filtered and concentrated in vacuo. The residue was purified by silica gel column chromatography, eluted with PE/EA (1:1) to afford 2-(4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (58 mg, 17% yield) as a white solid. LCMS (ESI) [M+H]⁺: 508.

### Step 8: (S)-2-((S)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide and (R)-2-((S)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide and (S)-2-((R)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide and (R)-2-((R)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide

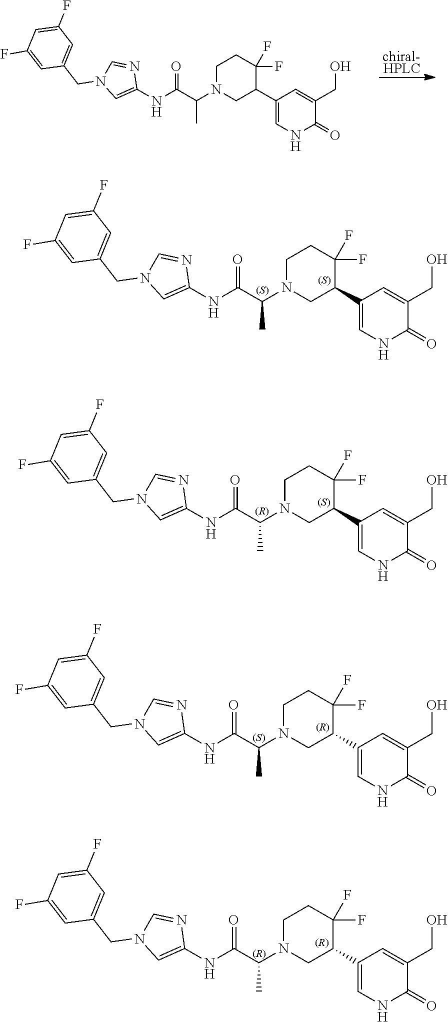

2-(4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (58 mg) was separated by prep-Chiral-HPLC with the following conditions Column: CHIRAL ART Cellulose-SC, 3×25 cm, 5 μm; Mobile Phase A: MtBE (10 mM NH₃-MeOH), Mobile Phase B: MEOH; Flow rate: 40 mL/min; Gradient: isocratic 30% B; Wave Length: 240/250 nm; RT1 (min): 5.68; RT2 (min): 7.4575; RT3 (min): 13.73; RT4 (min): 20.28) to afford (S)-2-((S)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (Compound 71) (Method R, 1.12 min, peak 1, 5.9 mg, 100% e.e.) as a white solid, (S)-2-((R)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (Compound 73) (Method R, 3.57 min, peak 3, 9.5 mg, 100% e.e.) as a white solid, (R)-2-((S)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (Compound 72) (Method R, 1.88 min, peak 3, 7.4 mg, 100% e.e.) as a white solid and (R)-2-((R)-4,4-difluoro-3-(5-(hydroxymethyl)-6-oxo-1,6-dihydropyridin-3-yl)piperidin-1-yl)-N-(1-(3,5-difluorobenzyl)-1H-imidazol-4-yl)propanamide (Method R, 7.23 min, peak 4, Compound 74) (9.8 mg, 100% e.e.) as a white solid.

TABLE 21

| Compound# | Characterization Data |
|---|---|
| 71 | LCMS (ESI) [M + H]⁺: 508.1 |
| | ¹H NMR (400 MHz, DMSO-d₆) δ 11.52 (s, 1H), 10.18 (s, 1H), 7.63 (d, J = 1.5 Hz, 1H), 7.40 (s, |
| | 1H), 7.30 (d, J = 1.5 Hz, 1H), 7.26 − 7.16 (m, 2H), 7.10 − 7.00 (m, 2H), 5.16 (s, 2H), 5.09 − 5.03 |
| | (m, 1H), 4.26 (d, J = 5.4 Hz, 2H), 3.53 − 3.46 (m, 1H), 3.24 − 3.11 (m, 1H), 3.01 − 2.86 (m, 2H), |
| | 2.69 − 2.56 (m, 2H), 2.16 − 1.93 (m, 2H), 1.17 (d, J = 6.9 Hz, 3H). |
| 72 | LCMS (ESI) [M + H]⁺: 508.12 |
| | ¹H NMR (400 MHz, DMSO-d₆) δ 11.54 (s, 1H), 10.19 (s, 1H), 7.64 (d, J = 1.5 Hz, 1H), 7.42 (s, |
| | 1H), 7.32 (d, J = 1.6 Hz, 1H), 7.25 − 7.16 (m, 2H), 7.10 − 7.04 (m, 2H), 5.16 (s, 2H), 5.13 − 5.03 |
| | (m, 1H), 4.27 (d, J = 5.4 Hz, 2H), 3.60 − 3.50 (m, 1H), 3.22 − 3.06 (m, 1H), 2.89 − 2.65 (m, 3H), |
| | 2.49 − 2.43 (m, 1H), 2.17 − 1.93 (m, 2H), 1.16 (d, J = 6.9 Hz, 3H). |
| 73 | LCMS (ESI) [M + H]⁺: 508.12 |
| | ¹H NMR (400 MHz, DMSO-d₆) δ 11.52 (s, 1H), 10.18 (s, 1H), 7.63 (d, J = 1.5 Hz, 1H), 7.40 (s, |
| | 1H), 7.30 (d, J = 1.5 Hz, 1H), 7.25 − 7.19 (m, 2H), 7.12 − 7.03 (m, 2H), 5.16 (s, 2H), 5.09 − 5.03 |
| | (m, 1H), 4.26 (d, J = 5.4 Hz, 2H), 3.57 − 3.46 (m, 1H), 3.18 − 3.05 (m, 1H), 2.98 − 2.87 (m, 2H), |
| | 2.72 − 2.56 (m, 2H), 2.16 − 1.92 (m, 2H), 1.17 (d, J = 6.8 Hz, 3H). |
| 74 | LCMS (ESI) [M + H]⁺: 508.12 |
| | ¹H NMR (400 MHz, DMSO-d₆) δ 11.54 (s, 1H), 10.19 (s, 1H), 7.64 (d, J = 1.6 Hz, 1H), 7.42 (s, |
| | 1H), 7.32 (d, J = 1.5 Hz, 1H), 7.24 − 7.15 (m, 2H), 7.10 − 7.03 (m, 2H), 5.16 (s, 2H), 5.13 − 5.03 |
| | (m, 1H), 4.27 (d, J = 5.4 Hz, 2H), 3.55 − 3.51 (m, 1H), 3.21 − 3.07 (m, 1H), 2.89 − 2.78 (m, 3H), |
| | 2.48 − 2.39 (m, 1H), 2.11 − 1.96 (m, 2H), 1.16 (d, J = 6.9 Hz, 3H). |

### Example 48: 4-((S)-1-((S)-1-((1-(3,5-difluorophenyl)-1H-imidazol-4-yl)amino)-1-oxopropan-2-yl)-4,4-difluoropiperidin-3-yl)pyridine 1-oxide

### Step 1: 4-((S)-1-((S)-1-((1-(3,5-difluorophenyl)-1H-imidazol-4-yl)amino)-1-oxopropan-2-yl)-4,4-difluoropiperidin-3-yl)pyridine 1-oxide

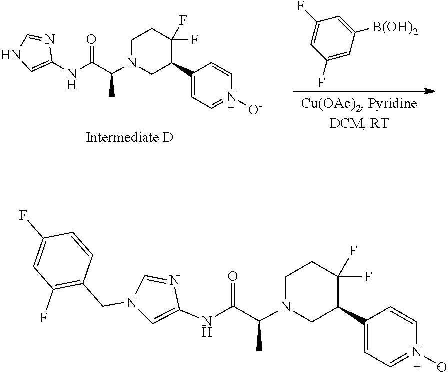

A solution of 4-((S)-1-((S)-1-((1H-imidazol-4-yl)amino)-1-oxopropan-2-yl)-4,4-difluoropiperidin-3-yl)pyridine 1-oxide (Intermediate D) (30 mg, 0.08 mmol), 3,5-difluorophenylboronic acid (137 mg, 0.86 mmol) and Cu(OAc)₂ (31 mg, 0.17 mmol) in DCM (0.5 mL) and pyridine (0.5 mL) was stirred at room temperature for 2 hours under a oxygen atmosphere. The resulting mixture was concentrated under reduced pressure. The crude product was purified by Prep-HPLC with the following conditions (Column: XSelect CSH Prep C18 OBD Column, 30×150 mm, 5 μm; Mobile Phase A: Water (0.1% FA), Mobile Phase B: ACN; Flow rate: 60 mL/min; Gradient: 16% B to 36% B in 9 min; Wave Length: 254 nm/220 nm; RT1 (min): 9.38) to afford 4-((S)-1-((S)-1-((1-(3,5-difluorophenyl)-1H-imidazol-4-yl)amino)-1-oxopropan-2-yl)-4,4-difluoropiperidin-3-yl)pyridine 1-oxide (Compound 75) (9.5 mg, 24% yield) as a white solid.

LCMS (ESI) [M+H]⁺: 464.1

¹H NMR (400 MHz, DMSO-d₆) δ 10.42 (s, 1H), 8.28 (d, J=1.6 Hz, 1H), 8.18 (d, J=7.0 Hz, 2H), 7.87 (d, J=1.7 Hz, 1H), 7.63-7.54 (m, 2H), 7.38 (d, J=6.7 Hz, 2H), 7.27-7.18 (m, 1H), 3.63-3.59 (m, 1H), 3.36-3.30 (m, 2H), 3.10-2.91 (m, 3H), 2.57-2.49 (m, 1H), 2.19-2.00 (m, 2H), 1.22 (d, J=6.9 Hz, 3H).

### Example 49: 2-(4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide

### Step 1: tert-butyl 3-(6-methoxypyridin-3-yl)-4-oxopiperidine-1-carboxylate

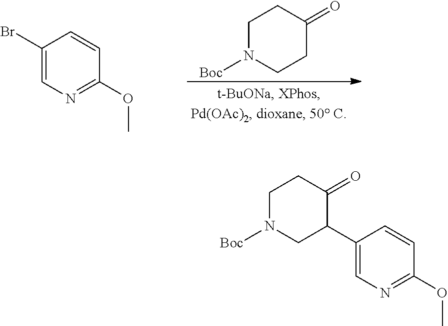

A mixture of 5-bromo-2-methoxypyridine (1.0 g, 5.31 mmol), tert-butyl 4-oxopiperidine-1-carboxylate (1.3 g, 6.52 mmol), t-BuONa (1.5 g, 15.60 mmol), XPHOS (0.5 g, 1.04 mmol) and Pd(OAc)₂ (0.1 g, 0.44 mmol) in dioxane (20 mL) was stirred for 1 hour at 50° C. under a nitrogen atmosphere. The mixture was allowed to cool down to room temperature. The resulting mixture was filtered, the filter cake was washed with EtOAc (3×25 mL). The resulting mixture was concentrated under reduced pressure. The residue was purified by silica gel column chromatography, eluted with CH₂Cl₂/EA (3:1) to afford tert-butyl 3-(6-methoxypyridin-3-yl)-4-oxopiperidine-1-carboxylate (500 mg, 30% yield) as a yellow oil. LCMS (ESI) [M+H]⁺: 307.

### Step 2: tert-butyl 4,4-difluoro-3-(6-methoxypyridin-3-yl)piperidine-1-carboxylate

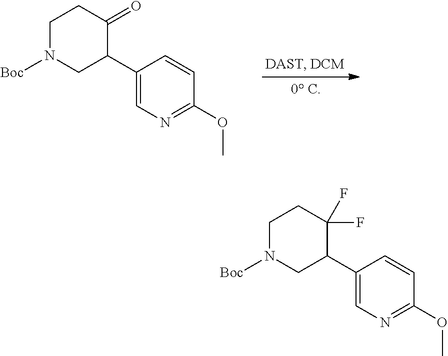 [SMILES: COc1ccc(C2CN(C(=O)OC(C)(C)C)CCC2=O)cn1 ; COc1ccc(C2CN(C(=O)OC(C)(C)C)CCC2(F)F)cn1]

To a mixture of tert-butyl 3-(6-methoxypyridin-3-yl)-4-oxopiperidine-1-carboxylate (490 mg, 1.60 mmol) in DCM (5 mL) was added DAST (516 mg, 3.20 mmol) dropwise at 0° C. under a nitrogen atmosphere. The final reaction mixture was stirred at room temperature for 3 hours. The reaction was quenched by the addition of sat. aq. sodium hyposulfite (12 mL) at 0° C. The resulting mixture was extracted with EtOAc (3×15 mL). The combined organic layers were dried over anhydrous Na₂SO₄ and filtered. The filtrate was concentrated under reduced pressure. The residue was purified by silica gel column chromatography, eluted with PE/EA (1:1) to afford tert-butyl 4,4-difluoro-3-(6-methoxypyridin-3-yl)piperidine-1-carboxylate (300 mg, 57% yield) as a white solid. LCMS (ESI) [M+H]⁺: 329.

### Step 3: 5-(4,4-difluoropiperidin-3-yl)pyridin-2(1H)-one

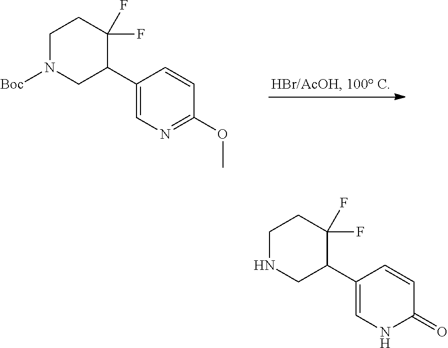 [SMILES: COc1ccc(C2CN(C(=O)OC(C)(C)C)CCC2(F)F)cn1 ; O=c1ccc(C2CNCCC2(F)F)c[nH]1]

A solution of tert-butyl 4,4-difluoro-3-(6-methoxypyridin-3-yl)piperidine-1-carboxylate (300 mg, 0.91 mmol) in HBr/AcOH (1 mL) was stirred for 1 hour at 100° C. under a nitrogen atmosphere. The mixture was allowed to cool down to room temperature. The mixture was concentrated under vacuum. The residue was purified by silica gel column chromatography, eluted with CH₂Cl₂/MeOH (1:1) to 5-(4,4-difluoropiperidin-3-yl)pyridin-2(1H)-one (150 mg, 76% yield) as a yellow oil. LCMS (ESI) [M+H]⁺: 215

### Step 4: 2-(4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide

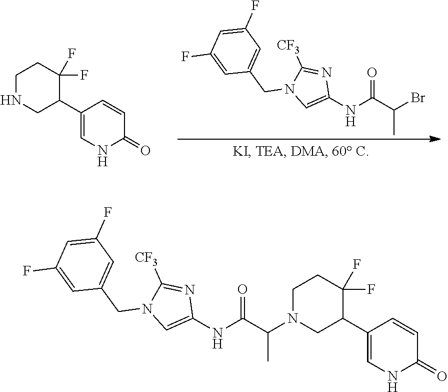 [SMILES: O=c1ccc(C2CNCCC2(F)F)c[nH]1 ; CC(Br)C(=O)Nc1cn(Cc2cc(F)cc(F)c2)c(C(F)(F)F)n1 ; CC(C(=O)Nc1cn(Cc2cc(F)cc(F)c2)c(C(F)(F)F)n1)N1CCC(F)(F)C(c2ccc(=O)[nH]c2)C1]

A solution of 5-(4,4-difluoropiperidin-3-yl)pyridin-2(1H)-one (60 mg, 0.28 mmol), 2-bromo-N-(1-(3,5-difluorobenzyl)-2-(trifluoromethyl)-1H-imidazol-4-yl)propanamide (116 mg, 0.33 mmol), KI (56 mg, 0.33 mmol) and TEA (85 mg, 0.84 mmol) in DMA (1 mL) was stirred for 1 hour at 60° C. under N₂ atmosphere. The mixture was allowed to cool down to room temperature. The residue was dissolved in water (3 mL). The resulting mixture was extracted with EtOAc (3×5 mL). The combined organic layers were washed with water (3×6 mL), dried over anhydrous Na₂SO₄ and filtered. The filtrate was concentrated under vacuum. The crude product was purified by Prep-HPLC with the following conditions (Column: XBridge Shield RP18 OBD Column 30×150 mm, 5 μm; Mobile Phase A: Water (10 mmol/L NH₄HCO₃), Mobile Phase B: ACN; Flow rate: 60 mL/min; Gradient: 5% B to 5% B in 1.5 min, 5% B to 20% B in 2 min, 20% B to 41% B in 9 min; Wave Length: 254 nm/220 nm; RT1 (min): 10.07) to afford 2-(4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide (64 mg, 52% yield) as a white solid. LCMS (ESI) [M+H]⁺: 546.

### Step 5: (S)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide, (R)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide, (2S)-2-[(3R)-4,4-difluoro-3-(1-methyl-6-oxopyridin-3-yl)piperidin-1-yl]-N-{1-[(3,5-difluorophenyl)methyl]imidazol-4-yl}propanamide, (R)-2-((R)-4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide

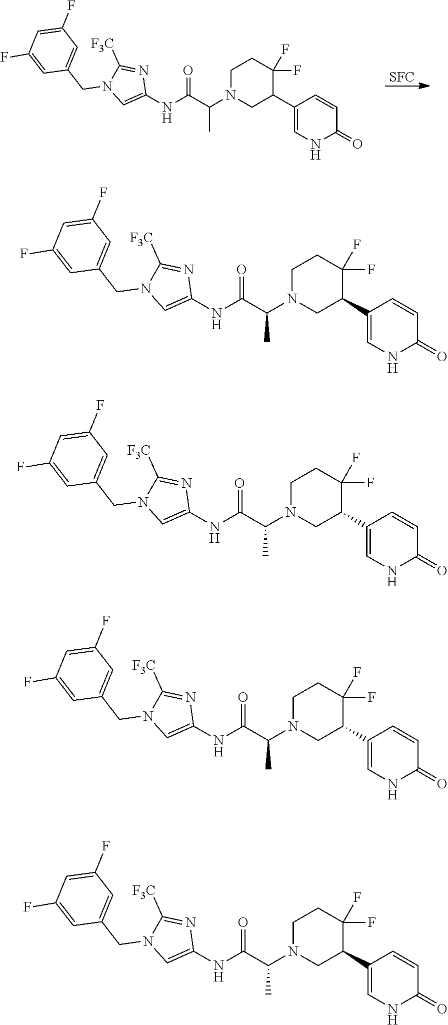 [SMILES: CC(C(=O)Nc1cn(Cc2cc(F)cc(F)c2)c(C(F)(F)F)n1)N1CCC(F)(F)C(c2ccc(=O)[nH]c2)C1 ; "SFC" ; C[C@@H](C(=O)Nc1cn(Cc2cc(F)cc(F)c2)c(C(F)(F)F)n1)N1CCC(F)(F)[C@@H](c2ccc(=O)[nH]c2)C1 ; C[C@H](C(=O)Nc1cn(Cc2cc(F)cc(F)c2)c(C(F)(F)F)n1)N1CCC(F)(F)[C@H](c2ccc(=O)[nH]c2)C1 ; C[C@@H](C(=O)Nc1cn(Cc2cc(F)cc(F)c2)c(C(F)(F)F)n1)N1CCC(F)(F)[C@H](c2ccc(=O)[nH]c2)C1 ; C[C@H](C(=O)Nc1cn(Cc2cc(F)cc(F)c2)c(C(F)(F)F)n1)N1CCC(F)(F)[C@@H](c2ccc(=O)[nH]c2)C1]

2-(4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide (64 mg) was separated by reversed-phase flash chromatography with the following conditions: column, C18; mobile phase, MeCN in water (10 mmol/L NH₄HCO₃), 5% to 100% gradient in 20 min to give two portions (28 mg, RT1: 8 min, 26 mg, RT2: 11 min), each as a mixture of diastereomers. The first portion was separated by prep-Chiral-HPLC Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 30% B; Wave Length: 207/231 nm; RT1 (min): 7.2; RT2 (min): 8.8; Sample Solvent: ETOH:DCM=2:1; Number Of Runs: 4 to afford (S)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (Method S, 0.82 min, peak 1, Compound 76) (12.4 mg, 100% e.e.) (RT1) and (R)-2-((R)-4,4-difluoro-3-(6-oxo-1,6-dihydropyridin-3-yl)piperidin-1-yl)-N-(1-(3,5-difluorobenzyl)-2-(trifluoromethyl)-1H-imidazol-4-yl)propanamide (Compound 77) (Method S, 1.03 min, peak 2, 10.7 mg, 100% e.e.) (RT2). The second portion was separated by Column: CHIRALPAK IE, 3*25 cm, 5 μm; Mobile Phase A: MtBE (10 mM NH₃-MeOH), Mobile Phase B: MeOH; Flow rate: 40 mL/min; Gradient: isocratic 20% B; Wave Length: 214/272 nm; RT1 (min): 9.87; RT2 (min): 16.12; Sample Solvent: MeOH:DCM=1:1; Injection Volume: 2.0 mL; Number Of Runs: 2 to yield (2S)-2-[(3R)-4,4-difluoro-3-(1-methyl-6-oxopyridin-3-yl)piperidin-1-yl]-N-{1-[(3,5-difluorophenyl)methyl]imidazol-4-yl}propanamide (Compound 78) (Method S, 1.88 min, peak 1, 10.5 mg, 100% e.e.) (RT1) and (R)-2-((S)-4,4-difluoro-3-(6-oxo-1,6-dihydropyridin-3-yl)piperidin-1-yl)-N-(1-(3,5-difluorobenzyl)-1H-imidazol-4-yl)propanamide (Compound 79) (Method S, 2.52 min, peak 2, 14.9 mg, 100% e.e.) (RT 2).

TABLE 22

| Compound# | Characterization Data |
|---|---|
| 76 | LCMS (ESI) [M + H]⁺: 546.3 |
| | ¹H NMR (400 MHz, Methanol-d₄) δ 7.67 − 7.60 (m, 1H), 7.60 (s, 1H), 7.43 (d, J = 2.5 Hz, 1H), |
| | 6.94 (tt, J = 9.1, 2.3 Hz, 1H), 6.85 − 6.78 (m, 2H), 6.52 (d, J = 9.4 Hz, 1H), 5.39 (s, 2H), 3.63 − |
| | 3.52 (m, 1H), 3.42 − 3.33 (m, 1H), 3.21 − 2.64 (m, 4H), 2.29 − 2.06 (m, 2H), 1.37 (d, J = 6.9 Hz, |
| | 3H) |
| 77 | LCMS (ESI) [M + H]⁺: 546.3 |
| | ¹H NMR (400 MHz, Methanol-d₄) δ 7.66 − 7.58 (m, 2H), 7.43 (d, J = 2.6 Hz, 1H), 7.01 − 6.88 |
| | (m, 1H), 6.84 − 6.75 (m, 2H), 6.53 (d, J = 9.4 Hz, 1H), 5.39 (s, 2H), 3.87 − 3.64 (m, 1H), 3.45 − |
| | 3.32 (m, 2H), 3.29 − 3.17 (m, 2H), 2.99 − 2.76 (m, 1H), 2.43 − 2.20 (m, 2H), 1.39 (s, 3H) |
| 78 | LCMS (ESI) [M + H]⁺: 546.4 |
| | ¹H NMR (400 MHz, Methanol-d₄) δ 7.65 − 7.58 (m, 2H), 7.44 (d, J = 2.6 Hz, 1H), 6.94 (tt, J = |
| | 9.0, 2.3 Hz, 1H), 6.85 − 6.75 (m, 2H), 6.53 (d, J = 9.5 Hz, 1H), 5.39 (s, 2H), 3.83 (s, 1H), 3.46 − |
| | 3.31 (m, 4H), 2.96 (s, 1H), 2.48 − 2.19 (m, 2H), 1.51 (s, 3H). |
| 79 | LCMS (ESI) [M + H]⁺: 546.3 |
| | ¹H NMR (400 MHz, Methanol-d₄) δ 7.69 − 7.56 (m, 2H), 7.45 (d, J = 2.6 Hz, 1H), 6.94 (tt, J = |
| | 9.1, 2.3 Hz, 1H), 6.84 − 6.78 (m, 2H), 6.53 (d, J = 9.5 Hz, 1H), 5.39 (s, 2H), 3.81 − 3.65 (m, 1H), |
| | 3.49 − 3.33 (m, 1H), 3.28 − 3.15 (m, 2H), 3.10 − 2.91 (m, 2H), 2.39 − 2.17 (m, 2H), 1.44 (d, J = |
| | 6.8 Hz, 3H). |

### Example 50: 4-((3S)-1-(1-((2-chloro-1-(3,5-difluorobenzyl)-1H-imidazol-4-yl)amino)-1-oxopropan-2-yl)-4,4-difluoropiperidin-3-yl)pyridine 1-oxide

### Step 1: ethyl 1-(3,5-difluorobenzyl)-1H-imidazole-4-carboxylate

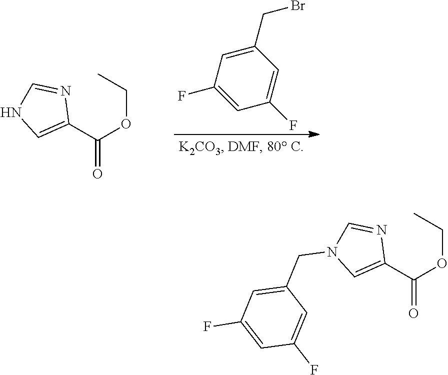

A mixture of ethyl 1H-imidazole-4-carboxylate (6.0 g, 42.1 mmol), 1-(bromomethyl)-3,5-difluorobenzene (10.0 g, 47.0 mmol) and K₂CO₃ (17.0 g, 128.4 mmol) in DMF (50 mL) was stirred for 2 hours at 80° C. under a nitrogen atmosphere. The mixture was allowed to cool down to room temperature. The reaction was quenched with water (50 mL) at room temperature. The resulting mixture was extracted with EtOAc (3×70 mL). The combined organic layers were washed with water (3×80 mL), dried over anhydrous Na₂SO₄ and filtered. The filtrate was concentrated under vacuum. The residue was purified by silica gel column chromatography, eluted with PE/EA (1:1) to afford ethyl 1-(3,5-difluorobenzyl)-1H-imidazole-4-carboxylate (1.9 g, 17% yield) as a white solid. LCMS (ESI) [M+H]⁺: 267.

### Step 2: ethyl 2-chloro-1-(3,5-difluorobenzyl)-1H-imidazole-4-carboxylate

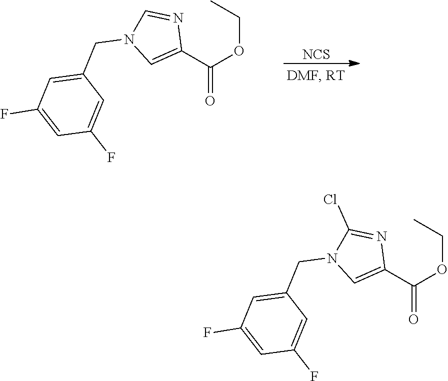 [SMILES: CCOC(=O)c1cn(Cc2cc(F)cc(F)c2)cn1 ; "NCS" ; CCOC(=O)c1cn(Cc2cc(F)cc(F)c2)c(Cl)n1]

A mixture of ethyl 1-(3,5-difluorobenzyl)-1H-imidazole-4-carboxylate (1.3 g, 4.81 mmol) and NCS (717 mg, 5.33 mmol) in DMF (8 mL) was stirred for 2 hours at 45° C. under a nitrogen atmosphere. The mixture was allowed to cool down to room temperature. The residue was purified by reversed-phase flash chromatography with the following conditions: column, C18 silica gel; mobile phase, MeCN in Water (10 mmol/L NH₄HCO₃), 0% to 100% gradient in 20 min; detector, UV 254 nm to afford ethyl 2-chloro-1-(3,5-difluorobenzyl)-1H-imidazole-4-carboxylate (890 mg, 61% yield) as a white solid. LCMS (ESI) [M+H]⁺: 301.

### Step 3: 2-chloro-1-(3,5-difluorobenzyl)-1H-imidazole-4-carboxylic acid

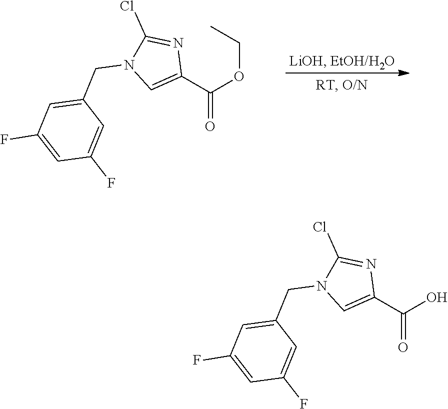 [SMILES: CCOC(=O)c1cn(Cc2cc(F)cc(F)c2)c(Cl)n1 ; "LiOH, EtOH/H2O" ; O=C(O)c1cn(Cc2cc(F)cc(F)c2)c(Cl)n1]

A mixture of ethyl 2-chloro-1-(3,5-difluorobenzyl)-1H-imidazole-4-carboxylate (890 mg, 2.93 mmol), LiOH (354 mg, 14.83 mmol) in 1:1 EtOH:H₂O (10 mL) was stirred for 2 hours at room temperature. The mixture was acidified to pH 4 with 2M aq. HCl. The resulting mixture was concentrated under vacuum. The residue was purified by reversed-phase flash chromatography with the following conditions: column, C18 silica gel; mobile phase, MeCN in Water (0.1% FA), 0% to 100% gradient in 20 min; detector, UV 254 nm to afford 2-chloro-1-(3,5-difluorobenzyl)-1H-imidazole-4-carboxylic acid (865 mg, crude) as a white solid. LCMS (ESI) [M+H]⁺: 273.

### Step 4: tert-butyl (2-chloro-1-(3,5-difluorobenzyl)-1H-imidazol-4-yl)carbamate

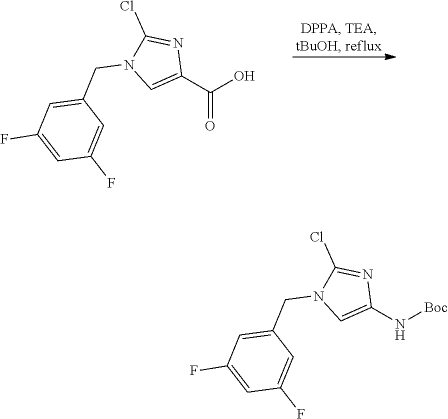 [SMILES: O=C(O)c1cn(Cc2cc(F)cc(F)c2)c(Cl)n1 ; "DPPA, TEA,tBuOH, reflux" ; CC(C)(C)OC(=O)Nc1cn(Cc2cc(F)cc(F)c2)c(Cl)n1]

A solution of 2-chloro-1-(3,5-difluorobenzyl)-1H-imidazole-4-carboxylic acid (860 mg, 3.1 mmol) in t-BuOH (6 mL) was treated with TEA (1.3 g, 12.6 mmol) for 1 hour at room temperature under a nitrogen atmosphere followed by the addition of DPPA (2.6 g, 9.4 mmol) dropwise at 0° C. The resulting mixture was stirred for 2 hours at 80° C. under a nitrogen atmosphere. The mixture was allowed to cool down to room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 0% to 100% gradient in 20 min; detector, UV 254 nm to afford tert-butyl (2-chloro-1-(3,5-difluorobenzyl)-1H-imidazol-4-yl)carbamate (300 mg, 28% yield) as a light yellow solid. LCMS (ESI) [M+H]⁺: 344.

### Step 5: tert-butyl (2-bromopropanoyl)(2-chloro-1-(3,5-difluorobenzyl)-1H-imidazol-4-yl)carbamate

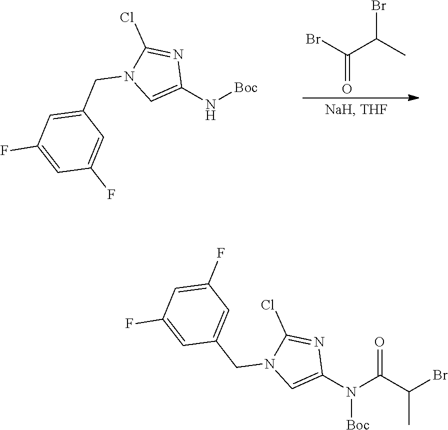

A solution of tert-butyl (2-chloro-1-(3,5-difluorobenzyl)-1H-imidazol-4-yl)carbamate (290 mg, 0.84 mmol) in THF (3 mL) was treated with NaH (60% in mineral oil, 40 mg, 1.7 mmol) at 0° C. The resulting mixture was stirred for 30 minutes at 0° C. To the reaction mixture was added 2-bromopropanoyl bromide (273 mg, 1.3 mmol) dropwise at 0° C. The resulting mixture was stirred for additional 2 hours at room temperature under a nitrogen atmosphere. The reaction was quenched with the addition of MeOH (10 mL) at 0° C. The resulting mixture was concentrated under vacuum. The residue was purified by reversed-phase flash chromatography with the following conditions: column, C18 silica gel; mobile phase, MeCN in Water (0.1% FA), 0% to 100% gradient in 30 min; detector, UV 254 nm to afford tert-butyl (2-bromopropanoyl)(2-chloro-1-(3,5-difluorobenzyl)-1H-imidazol-4-yl)carbamate (190 mg, 37% yield) as a light yellow solid. LCMS (ESI) [M+H]⁺: 478.

### Step 6: 4-((3S)-1-(1-((tert-butoxycarbonyl)(2-chloro-1-(3,5-difluorobenzyl)-1H-imidazol-4-yl)amino)-1-oxopropan-2-yl)-4,4-difluoropiperidin-3-yl)pyridine 1-oxide

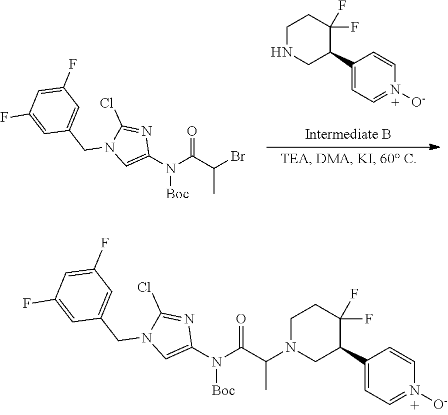

A mixture of tert-butyl (2-bromopropanoyl)(2-chloro-1-(3,5-difluorobenzyl)-1H-imidazol-4-yl)carbamate (185 mg, 0.38 mmol), (S)-4-(4,4-difluoropiperidin-3-yl)pyridine 1-oxide (Intermediate B) (82 mg, 0.38 mmol), KI (77 mg, 0.46 mmol) and TEA (117 mg, 1.15 mmol) in DMA (1.5 mL) was stirred for 4 hours at 60° C. under a nitrogen atmosphere. The mixture was allowed to cool down to room temperature. The residue was purified by reversed-phase flash chromatography with the following conditions: column, C18 silica gel; mobile phase, MeCN in Water (0.1% FA), 0% to 100% gradient in 30 min; detector, UV 254 nm to afford 4-((3S)-1-(1-((tert-butoxycarbonyl)(2-chloro-1-(3,5-difluorobenzyl)-1H-imidazol-4-yl)amino)-1-oxopropan-2-yl)-4,4-difluoropiperidin-3-yl)pyridine 1-oxide (110 mg, 47% yield) as a light yellow solid. LCMS (ESI) [M+H]⁺:612.

### Step 7: 4-((3S)-1-(1-((2-chloro-1-(3,5-difluorobenzyl)-1H-imidazol-4-yl)amino)-1-oxopropan-2-yl)-4,4-difluoropiperidin-3-yl)pyridine 1-oxide

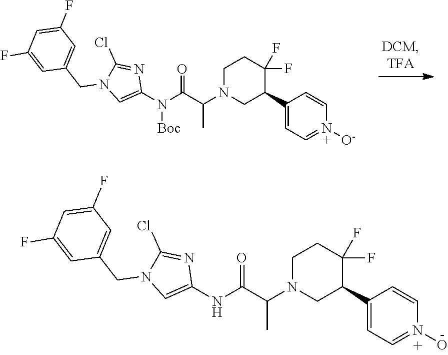

A mixture of 4-((3S)-1-(1-((tert-butoxycarbonyl)(2-chloro-1-(3,5-difluorobenzyl)-1H-imidazol-4-yl)amino)-1-oxopropan-2-yl)-4,4-difluoropiperidin-3-yl)pyridine 1-oxide (110 mg, 0.18 mmol, 1 equiv) in DCM (0.5 mL) and TFA (0.5 mL) was stirred for 24 h at room temperature. The resulting mixture was concentrated under vacuum. The residue was purified by reversed-phase flash chromatography with the following conditions: column, C18 silica gel; mobile phase, MeCN in Water (0.1% FA), 0% to 100% gradient in 30 min; detector, UV 254 nm to afford 4-((3S)-1-(1-((2-chloro-1-(3,5-difluorobenzyl)-1H-imidazol-4-yl)amino)-1-oxopropan-2-yl)-4,4-difluoropiperidin-3-yl)pyridine 1-oxide (54 mg, 35% yield) as a light yellow oil.

LCMS (ESI) [M+H]⁺:512.

### Step 8: 4-((S)-1-((S)-1-((2-chloro-1-(3,5-difluorobenzyl)-1H-imidazol-4-yl)amino)-1-oxopropan-2-yl)-4,4-difluoropiperidin-3-yl)pyridine 1-oxide and 4-((S)-1-((R)-1-((2-chloro-1-(3,5-difluorobenzyl)-1H-imidazol-4-yl)amino)-1-oxopropan-2-yl)-4,4-difluoropiperidin-3-yl)pyridine 1-oxide

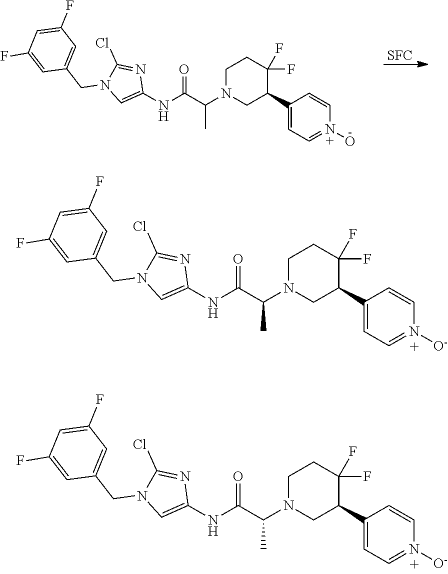

4-((3S)-1-(1-((2-chloro-1-(3,5-difluorobenzyl)-1H-imidazol-4-yl)amino)-1-oxopropan-2-yl)-4,4-difluoropiperidin-3-yl)pyridine 1-oxide (54 mg) was purified by SFC with the following conditions (Column: CHIRAL ART Cellulose-SC, 3×25 cm, 5 μm; Mobile Phase A: MtBE (10 mM NH₃-MeOH), Mobile Phase B: MEOH; Flow rate: 40 mL/min; Gradient: isocratic 50% B; Wave Length: 212/270 nm; RT1 (min): 7.21; RT2 (min): 14.27; Sample Solvent: MEOH; Injection Volume: 2.0 mL; Number Of Runs: 2) to afford 4-((S)-1-((S)-1-((2-chloro-1-(3,5-difluorobenzyl)-1H-imidazol-4-yl)amino)-1-oxopropan-2-yl)-4,4-difluoropiperidin-3-yl)pyridine 1-oxide (Compound 80) (Method B, 1.25 min, peak 1, 17.4 mg, 100% e.e.) as a white solid and 4-((S)-1-((R)-1-((2-chloro-1-(3,5-difluorobenzyl)-1H-imidazol-4-yl)amino)-1-oxopropan-2-yl)-4,4-difluoropiperidin-3-yl)pyridine 1-oxide (Compound 81) (Method B, 2.46 min, peak 2, 13.5 mg, 100% e.e.) as a white solid.

TABLE 23

| Compound# | Characterization Data |
|---|---|
| 80 | LCMS (ESI) [M + H]⁺: 512.1 |
| | ¹H NMR (400 MHz, DMSO-d₆) δ 9.59 (s, 1H), 8.18 (d, J = 7.2 Hz, 2H), 7.90 (s, 1H), 7.35 (d, J = |
| | 6.9 Hz, 2H), 7.29 − 7.16 (m, 1H), 7.00 − 6.82 (m, 2H), 5.26 (s, 2H), 3.65 − 3.44 (m, 2H), 2.94 − |
| | 2.62 (m, 4H), 2.23 − 2.06 (m, 2H), 1.19 (d, J = 6.9 Hz, 3H). |
| 81 | LCMS (ESI) [M + H]⁺: 512.1 |
| | ¹H NMR (400 MHz, DMSO-d₆) δ 9.62 (s, 1H), 8.18 (d, J = 7.2 Hz, 2H), 7.91 (s, 1H), 7.36 (d, J = |
| | 6.7 Hz, 2H), 7.28 − 7.17 (m, 1H), 6.93 (d, J = 4.6 Hz, 2H), 5.26 (s, 2H), 3.63 − 3.43 (m, 2H), |
| | 3.05 − 2.79 (m, 3H), 2.65 − 2.52 (m, 1H), 2.22 − 2.03 (m, 2H), 1.18 (d, J = 6.9 Hz, 3H). |

### Example 51: 4-((3S)-1-(1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide

### Step 1: methyl 4-bromo-1-(3,5-difluorobenzyl)-1H-imidazole-2-carboxylate

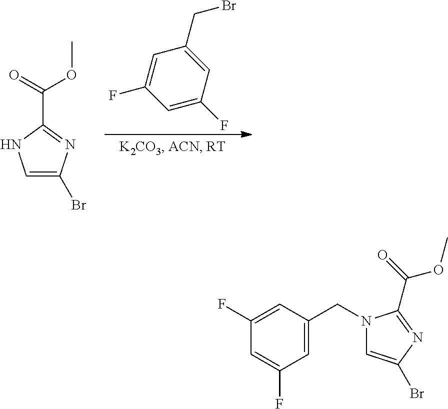

To a mixture of methyl 4-bromo-1H-imidazole-2-carboxylate (1.0 g, 4.9 mmol) and K₂CO₃ (2.0 g, 14.6 mmol) in ACN (15 mL) was added 1-(bromomethyl)-3,5-difluorobenzene (1.2 g, 5.9 mmol) in portions at room temperature. The resulting mixture was stirred for 1 hour at room temperature. The reaction was quenched with water (20 mL). The resulting mixture was extracted with EtOAc (3×20 mL), dried over anhydrous Na₂SO₄ and filtered. The filtrate was concentrated under vacuum. The residue was purified by silica gel column chromatography, eluted with PE/EA (1:1) to afford methyl 4-bromo-1-(3,5-difluorobenzyl)-1H-imidazole-2-carboxylate (1.4 g, 78% yield) as a yellow solid. LCMS (ESI) [M+H]⁺: 331.

### Step 2: 4-bromo-1-(3,5-difluorobenzyl)-1H-imidazole-2-carbaldehyde

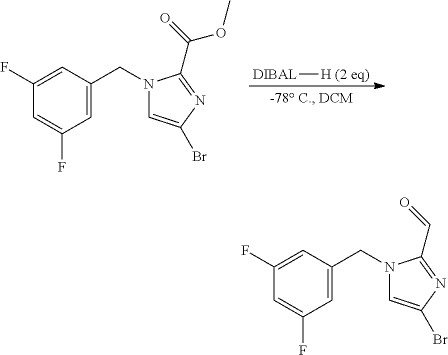

To a mixture of methyl 4-bromo-1-(3,5-difluorobenzyl)-1H-imidazole-2-carboxylate (1.3 g, 3.93 mmol) in DCM (25 mL) was added 1 M DIBAL-H in n-Hexane (7.86 mL, 7.86 mmol) dropwise at −78° C. under a nitrogen atmosphere. The resulting mixture was stirred for 1 hour at −78° C. under nitrogen atmosphere. The reaction was quenched with sat. aq. NH₄Cl (20 mL) at −78° C. The resulting mixture was extracted with EtOAc (3×30 mL), dried over anhydrous Na₂SO₄, and filtered. The filtrate was concentrated under vacuum. The residue was purified by silica gel column chromatography, eluted with PE/EA (1:2) to afford 4-bromo-1-(3,5-difluorobenzyl)-1H-imidazole-2-carbaldehyde (800 mg, 61% yield) as a yellow oil. LCMS (ESI) [M+H]⁺: 301.

### Step 3: 4-bromo-1-(3,5-difluorobenzyl)-2-(difluoromethyl)-1H-imidazole

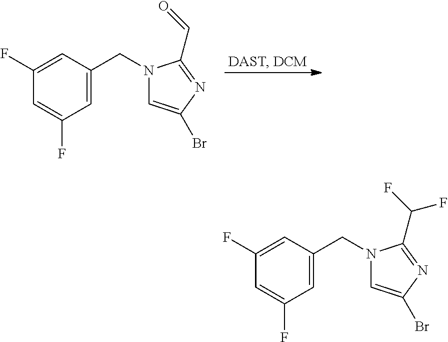

To a mixture of 4-bromo-1-(3,5-difluorobenzyl)-1H-imidazole-2-carbaldehyde (790 mg, 2.6 mmol) in DCM (10 mL) was added DAST (846 mg, 5.3 mmol) in portions at 0° C. under a nitrogen atmosphere. The resulting mixture was stirred for 1 hour at room temperature under a nitrogen atmosphere. The reaction was quenched with sat. aq. Na₂S₂O₃ (10 mL) at 0° C. The resulting mixture was extracted with EtOAc (3×20 mL), dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% FA), 0% to 100% gradient in 25 min; detector, UV 254 nm. This resulted in 4-bromo-1-(3,5-difluorobenzyl)-2-(difluoromethyl)-1H-imidazole (500 mg, 46% yield) as a light yellow solid. LCMS (ESI) [M+H]⁺: 323.

### Step 4: N-(1-(3,5-difluorobenzyl)-2-(difluoromethyl)-1H-imidazol-4-yl)-2-hydroxypropanamide

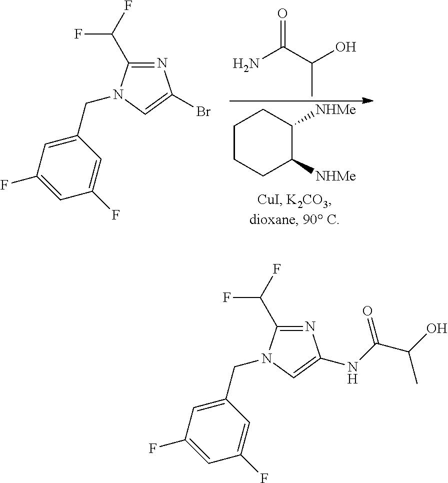

A mixture of 4-bromo-1-(3,5-difluorobenzyl)-2-(difluoromethyl)-1H-imidazole (490 mg, 1.3 mmol), lactamide (177 mg, 2.0 mmol), CuI (252 mg, 1.3 mmol), (1S,2S)—N1,N2-dimethylcyclohexane-1,2-diamine (187 mg, 1.32 mmol) and K₂CO₃ (549 mg, 3.97 mmol) in dioxane (10 mL) was stirred for 2 hours at 90° C. under a nitrogen atmosphere. The mixture was cooled to room temperature. The reaction was quenched with water (20 mL). The resulting mixture was extracted with EtOAc (3×15 mL), dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% FA), 0% to 100% gradient in 25 min; detector, UV 254 nm. This resulted in N-(1-(3,5-difluorobenzyl)-2-(difluoromethyl)-1H-imidazol-4-yl)-2-hydroxypropanamide (400 mg, 82% yield) as a yellow solid. LCMS (ESI) [M+H]⁺: 332.

### Step 5: 1-((1-(3,5-difluorobenzyl)-2-(difluoromethyl)-1H-imidazol-4-yl)amino)-1-oxopropan-2-yl 4-methylbenzenesulfonate

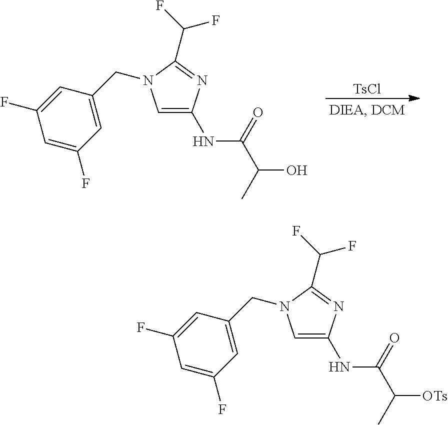 [SMILES: CC(O)C(=O)Nc1cn(Cc2cc(F)cc(F)c2)c(C(F)F)n1 ; "TsCl" ; Cc1ccc(S(=O)(=O)OC(C)C(=O)Nc2cn(Cc3cc(F)cc(F)c3)c(C(F)F)n2)cc1]

To a mixture of N-(1-(3,5-difluorobenzyl)-2-(difluoromethyl)-1H-imidazol-4-yl)-2-hydroxypropanamide (390 mg, 1.2 mmol) and DIEA (457 mg, 3.5 mmol) in DCM (10 mL) was added 4-methylbenzene-1-sulfonyl chloride (337 mg, 1.8 mmol) in portions at 0° C. The resulting mixture was stirred for 1 hour at room temperature. The reaction was quenched with water (10 mL). The resulting mixture was extracted with EtOAc (3×15 mL), dried over anhydrous Na₂SO₄ and filtered. The filtrate was concentrated under vacuum. The residue was purified by silica gel column chromatography, eluted with PE/EA (1:1) to afford 1-((1-(3,5-difluorobenzyl)-2-(difluoromethyl)-1H-imidazol-4-yl)amino)-1-oxopropan-2-yl 4-methylbenzenesulfonate (300 mg, 52% yield) as a light yellow solid. LCMS (ESI) [M+H]⁺: 486.

### Step 6: 4-((3S)-1-(1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide

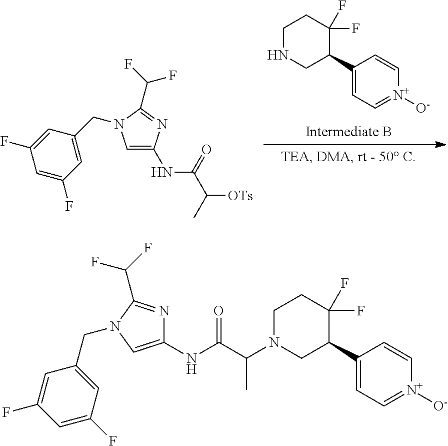 [SMILES: Cc1ccc(S(=O)(=O)OC(C)C(=O)Nc2cn(Cc3cc(F)cc(F)c3)c(C(F)F)n2)cc1 ; [O-][n+]1ccc([C@H]2CNCCC2(F)F)cc1 ; CC(C(=O)Nc1cn(Cc2cc(F)cc(F)c2)c(C(F)F)n1)N1CCC(F)(F)[C@@H](c2cc[n+]([O-])cc2)C1]

To a mixture of 1-((1-(3,5-difluorobenzyl)-2-(difluoromethyl)-1H-imidazol-4-yl)amino)-1-oxopropan-2-yl 4-methylbenzenesulfonate (100 mg, 0.21 mmol) and (S)-4-(4,4-difluoropiperidin-3-yl)pyridine 1-oxide (Intermediate B) (66 mg, 0.31 mmol) in DMA (5 mL) was added TEA (63 mg, 0.62 mmol) dropwise at room temperature. The resulting mixture was stirred for 2 hours at 50° C. The mixture was cooled to room temperature. The resulting mixture was concentrated under vacuum. The crude product was purified by Prep-HPLC with the following conditions (Column: XBridge Shield RP18 OBD Column, 5 um, 19×150 mm; Mobile Phase A: Water (10 MMOL/L NH₄HCO₃), Mobile Phase B: ACN; Flow rate: 25 mL/min; Gradient: 50% B to 80% B in 8 min; 220 nm; Rt: 8.2 min) to afford 4-((3S)-1-(1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide (35 mg, 32% yield) as a white solid. LCMS (ESI) [M+H]⁺: 528.

### Step 7: 4-((S)-1-((S)-1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide and 4-((S)-1-((R)-1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide

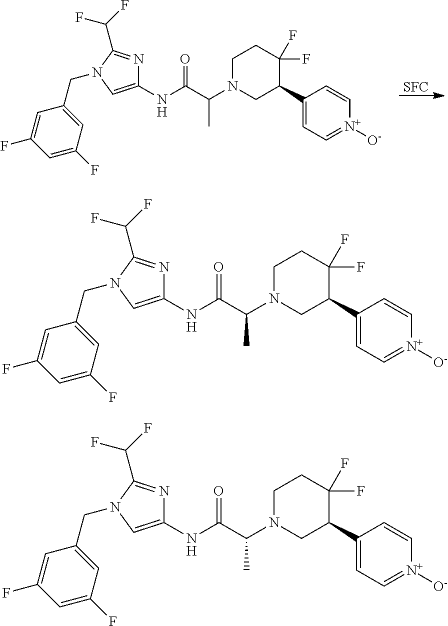

4-((3S)-1-(1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide (35 mg) was separated by prep-Chiral-HPLC (Column: CHIRAL ART Cellulose-SB, 3×25 cm, 5 μm; Mobile Phase A: MtBE (10 mM NH₃-MeOH), Mobile Phase B: MEOH; Flow rate: 40 mL/min; Gradient: isocratic 15% B; Wave Length: 272/216 nm; RT1 (min): 6.28; RT2 (min): 8.30) to afford 4-((S)-1-((S)-1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide (Compound 82) (Method T, 0.82 min, peak 1, 12.6 mg, 100% e.e.) and 4-((S)-1-((R)-1-((1-(3,5-difluorobenzyl)-2-(difluoromethyl)-1H-imidazol-4-yl)amino)-1-oxopropan-2-yl)-4,4-difluoropiperidin-3-yl)pyridine 1-oxide (Compound 83) (Method T, 1.07 min, peak 2, 10.7 mg, 99.7% e.e.). LCMS (ESI) [M+H]⁺: 528.15.

TABLE 24

| Compound# | Characterization Data |
|---|---|
| 82 | LCMS (ESI) [M + H]⁺: 528.2 |
| | ¹H NMR (400 MHz, DMSO-d₆) δ 10.45 (s, 1H), 8.15 (d, J = 6.8 Hz, 2H), 7.53 (s, 1H), 7.37 (d, J = |
| | 6.6 Hz, 2H), 7.26 − 7.16 (m, 2H), 7.00 − 6.89 (m, 2H), 5.36 (s, 2H), 3.62 − 3.40 (m, 2H), 2.99 − |
| | 2.91 (m, 2H), 2.77 − 2.66 (m, 2H), 2.21 − 1.96 (m, 2H), 1.20 (d, J = 6.9 Hz, 3H). |
| 83 | LCMS (ESI) [M + H]⁺: 528.1 |
| | ¹H NMR (400 MHz, DMSO-d₆) δ 10.44 (s, 1H), 8.17 (d, J = 6.8 Hz, 2H), 7.53 (s, 1H), 7.36 (d, J = |
| | 6.8 Hz, 2H), 7.27 − 7.17 (m, 2H), 7.01 − 6.89 (m, 2H), 5.36 (s, 2H), 3.66 − 3.42 (m, 2H), 3.01 − |
| | 2.81 (m, 3H), 2.54 − 2.50(m, 1H), 2.18 − 1.95 (m, 2H), 1.19 (d, J = 6.9 Hz, 3H). |

### Example 52: 4-((3S)-1-(1-(benzo[d]thiazol-2-ylamino)-1-oxopropan-2-yl)-4,4-difluoropiperidin-3-yl)pyridine 1-oxide

### Step 1: N-(benzo[d]thiazol-2-yl)-2-bromopropanamide

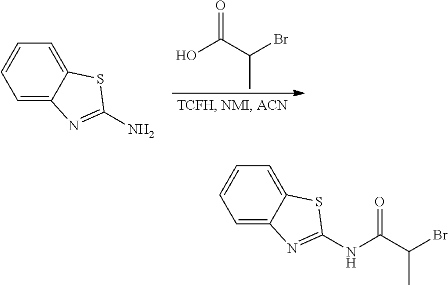

A mixture of benzo[d]thiazol-2-amine (150 mg, 998.7 μmol), 2-bromopropanoic acid (153 mg, 998.7 μmol), 1-methyl-1H-imidazole (130 mg, 1.6 mmol) and N-(chloro(dimethylamino)methylene)-N-methylmethanaminium hexafluorophosphate(V) (604 mg, 1.6 mmol) in ACN (3 mL) was stirred for overnight at room temperature. LCMS suggested complete consumption of the starting material. The resulting mixture was directly purified by reverse phase column chromatography, eluted with MeCN in water (0.1% formic acid), 5% to 50% gradient in 20 min; detector, UV 254 nm to afford N-(benzo[d]thiazol-2-yl)-2-bromopropanamide (177 mg, 62% yield) as a white solid. LCMS (ESI) [M+H]⁺: 284.96/286.96.

### Step 3: 4-((3S)-1-(1-(benzo[d]thiazol-2-ylamino)-1-oxopropan-2-yl)-4,4-difluoropiperidin-3-yl)pyridine 1-oxide

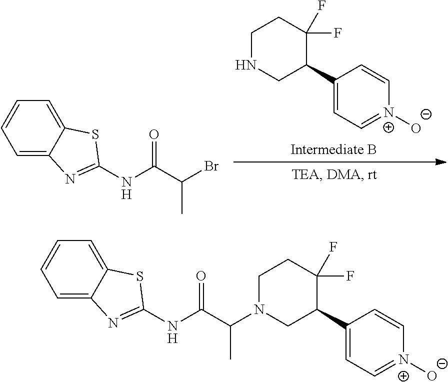 [SMILES: CC(Br)C(=O)Nc1nc2ccccc2s1 ; [O-][n+]1ccc([C@H]2CNCCC2(F)F)cc1 ; CC(C(=O)Nc1nc2ccccc2s1)N1CCC(F)(F)[C@@H](c2cc[n+]([O-])cc2)C1]

A mixture of N-(benzo[d]thiazol-2-yl)-2-bromopropanamide (80 mg, 227.9 μmol), (S)-4-(4,4-difluoropiperidin-3-yl)pyridine 1-oxide (Intermediate B) (49 mg, 227.9 μmol) and TEA (70 mg, 684.2 μmol) in DMA (1 mL) was stirred for 2 days at room temperature. The resulting mixture was poured into water (20 mL) and extracted with DCM (3×20 mL). Organic layers were separated, combined, dried over MgSO₄, filtered and concentrated. The crude product was purified by silica gel column chromatography, eluted with DCM/MeOH (90:10) to afford 4-((3S)-1-(1-(benzo[d]thiazol-2-ylamino)-1-oxopropan-2-yl)-4,4-difluoropiperidin-3-yl)pyridine 1-oxide (73.0 mg, 62% yield) as a white solid. LCMS (ESI) [M+H]⁺: 419.13.

### Step 4: 4-((S)-1-((S)-1-(benzo[d]thiazol-2-ylamino)-1-oxopropan-2-yl)-4,4-difluoropiperidin-3-yl)pyridine 1-oxide and 4-((S)-1-((R)-1-(benzo[d]thiazol-2-ylamino)-1-oxopropan-2-yl)-4,4-difluoropiperidin-3-yl)pyridine 1-oxide

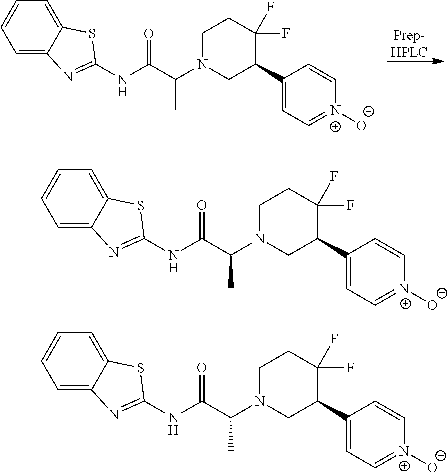 [SMILES: CC(C(=O)Nc1nc2ccccc2s1)N1CCC(F)(F)[C@@H](c2cc[n+]([O-])cc2)C1 ; "Prep-HPLC" ; C[C@@H](C(=O)Nc1nc2ccccc2s1)N1CCC(F)(F)[C@@H](c2cc[n+]([O-])cc2)C1 ; C[C@H](C(=O)Nc1nc2ccccc2s1)N1CCC(F)(F)[C@@H](c2cc[n+]([O-])cc2)C1]

4-((3S)-1-(1-(benzo[d]thiazol-2-ylamino)-1-oxopropan-2-yl)-4,4-difluoropiperidin-3-yl)pyridine 1-oxide (73 mg) was separated by Prep-HPLC with the following conditions Column: XSelect CSH Prep C18 OBD Column, 30×150 mm, 5 μm; Mobile Phase A: Water (0.1% FA), Mobile Phase B: ACN; Flow rate: 60 mL/min; Gradient: 23% B to 39% B in 9 min; Wave Length: 254 nm/220 nm; RT1 (min): 9.72/10.65 to afford first peak 4-((S)-1-((S)-1-(benzo[d]thiazol-2-ylamino)-1-oxopropan-2-yl)-4,4-difluoropiperidin-3-yl)pyridine 1-oxide (Compound 84) (Method U, 1.64 min, peak 2, 17.2 mg, 98.9% e.e.) as a white solid and second peak 4-((S)-1-((R)-1-(benzo[d]thiazol-2-ylamino)-1-oxopropan-2-yl)-4,4-difluoropiperidin-3-yl)pyridine 1-oxide (Compound 85) (Method U, 2.03 min, peak 2, 14.5 mg, 98.5% e.e.) as a white solid.

TABLE 25

| Compound# | Characterization Data |
|---|---|
| 84 | LCMS (ESI) [M + H]⁺: 419.2 |
| | ¹H NMR (400 MHz, DMSO-d₆) δ 12.27 (s, 1H), 8.15 (d, J = 7.0 Hz, 2H), 7.98 (d, J = 7.6 Hz, |
| | 1H), 7.75 (d, J = 7.7 Hz, 1H), 7.45 (ddd, J = 8.3, 7.2, 1.3 Hz, 1H), 7.38 (d, J = 7.1 Hz, 2H), |
| | 7.32 (td, J = 7.6, 1.2 Hz, 1H), 3.75 (q, J = 6.9 Hz, 1H), 3.59-3.43 (m, 1H), 3.10-2.61 (m, |
| | 4H), 2.26-1.98 (m, 2H), 1.29 (d, J = 6.9 Hz, 3H). |
| 85 | LCMS (ESI) [M + H]⁺: 419.3 |
| | ¹H NMR (400 MHz, DMSO-d₆) δ 12.25 (s, 1H), 8.18 (d, J = 6.9 Hz, 2H), 7.99 (d, J = 7.9 Hz, |
| | 1H), 7.76 (d, J = 8.1 Hz, 1H), 7.50-7.42 (m, 1H), 7.40 (d, J = 6.6 Hz, 2H), 7.35-7.27 (m, |
| | 1H), 3.79 (q, J = 6.8 Hz, 1H), 3.66-3.47 (m, 1H), 3.05-2.81 (m, 4H), 2.65-2.54 (m, 1H), |
| | 2.20-2.03 (m, 2H), 1.28 (d, J = 6.9 Hz, 3H). |

### Example 53: 4-((3S)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

### Step 1: 5-(3,5-difluorophenyl)pyrrolidin-2-one

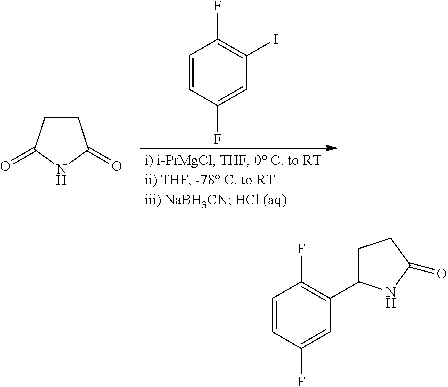 [SMILES: O=C1CCC(=O)N1 ; Fc1ccc(F)c(I)c1 ; O=C1CCC(c2cc(F)ccc2F)N1]

Isopropylmagnesium chloride lithium chloride complex (1.3 M in THF, 85.4 mL, 111.0 mmol) was added to a solution of 1,3-difluoro-5-iodobenzene (26.64 g, 111.0 mmol) in THF (250 mL) at −30° C. under nitrogen atmosphere. The mixture was warmed to room temperature and stirred for 0.5 h. Then the Grignard-exchange mixture was cooled to −78° C. and transferred to a mixture of succinimide (5.00 g, 50.5 mmol) in THF (250 mL) at −78° C. under nitrogen atmosphere. The mixture was slowly warmed and stirred at room temperature for 1.5 h. After that, the reaction mixture was cooled to 0° C. and NaBH₃CN (3.81 g, 60.6 mmol) was added in portions to the resulting mixture and stirred for 5 min. After that, the reaction mixture was acidified to pH=3-4 with aqueous HCl (6 M) at 0° C., stirred for 30 min at room temperature, and neutralized with ammonium hydroxide to pH 6-7. The reaction mixture diluted with water (300 mL) and extracted with ethyl acetate (3×350 mL). The combined organic layers were dried over anhydrous Na₂SO₄, filtered, and concentrated under reduced pressure. The crude residue was purified by silica gel chromatography (ethyl acetate/hexanes, 0-100%) to afford 5-(3,5-difluorophenyl)pyrrolidin-2-one (6.20 g, 62% yield) as a yellow solid.

LCMS (ESI) [M+H]⁺: 198.

¹H NMR (400 MHz, DMSO-d₆) δ 8.13 (s, 1H), 7.13 (tt, J=9.3, 2.4 Hz, 1H), 7.03 (d, J=4.3 Hz, 2H), 4.70 (t, J=7.1 Hz, 1H), 2.47-2.44 (m, 1H), 2.30-2.15 (m, 2H), 1.80-1.71 (m, 1H).

### Step 2: 2-(2-(3,5-difluorophenyl)-5-oxopyrrolidin-1-yl)acetamide

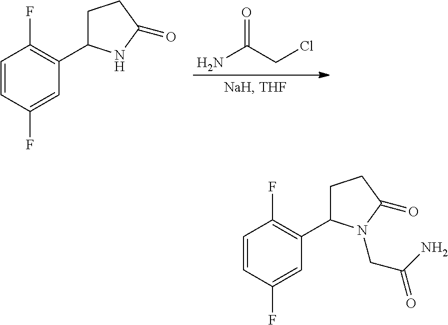 [SMILES: O=C1CCC(c2cc(F)ccc2F)N1 ; NC(=O)CCl ; NC(=O)CN1C(=O)CCC1c1cc(F)ccc1F]

To a solution of 5-(3,5-difluorophenyl)pyrrolidin-2-one (5.00 g, 25.4 mmol) and 2-chloroacetamide (4.74 g, 50.7 mmol) in anhydrous THF (50 mL) was added NaH (60% in mineral oil, 4.06 g, 101.4 mmol) at −20° C. The mixture was allowed to warm to room temperature and stirred for 18 h. After completion, the reaction mixture was diluted with DCM (100 mL) and quenched with saturated aqueous NH₄Cl solution (80 mL) at 0° C. The organic layer was combined and dried over anhydrous Na₂SO₄, filtered, and concentrated under reduced pressure. The crude residue was purified by silica gel chromatography (MeOH/DCM 0-8%) to afford 2-(2-(3,5-difluorophenyl)-5-oxopyrrolidin-1-yl)acetamide (3.60 g, 56% yield) as a light yellow solid.

LCMS (ESI) [M+H]⁺: 255.

### Step 3: 2-bromo-5-(3,5-difluorophenyl)-6,7-dihydro-5H-pyrrolo[1,2-a]imidazole

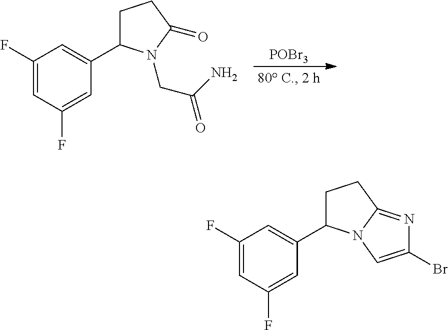

Phosphoryl bromide (11.28 g, 39.3 mmol) was added to 2-(2-(3,5-difluorophenyl)-5-oxopyrrolidin-1-yl)acetamide (2.50 g, 9.8 mmol) at room temperature. The reaction mixture was heated to 80° C. and stirred for 2 h. The mixture was allowed to cool down to room temperature. The resulting mixture was diluted with acetonitrile (50 mL). The reaction mixture was added dropwise to stirring warm water. The mixture was neutralized to pH 6-7 with ammonium hydroxide. The resulting mixture was extracted with EtOAc (3×80 mL). The combined organic layers were washed with brine, dried over anhydrous Na₂SO₄, filtered, and concentrated under reduced pressure. The crude residue was purified by silica gel column chromatography (ethyl acetate/petroleum ether 10%-70%) to afford 2-bromo-5-(3,5-difluorophenyl)-6,7-dihydro-5H-pyrrolo[1,2-a]imidazole (2.00 g, 68% yield) as a brown solid.

LCMS (ESI) [M+H]⁺: 299.

¹H NMR (400 MHz, DMSO-d₆) δ 7.27-7.20 (m, 1H), 7.17 (s, 1H), 6.93 (d, J=6.7, 3.4 Hz, 2H), 5.46 (s, 1H), 3.06-2.90 (m, 2H), 2.87-2.78 (m, 1H), 2.42-2.31 (m, 1H).

### Step 4: N-(5-(3,5-difluorophenyl)-6,7-dihydro-5H-pyrrolo[1,2-a]imidazol-2-yl)-2-hydroxypropanamide

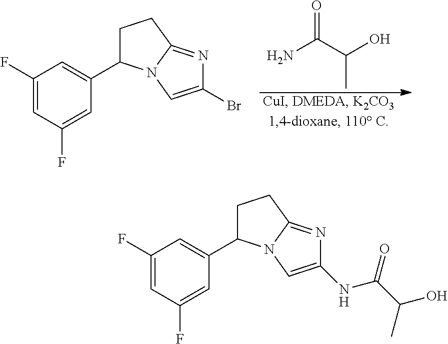

2-Bromo-5-(3,5-difluorophenyl)-6,7-dihydro-5H-pyrrolo[1,2-a]imidazole (1.20 g, 4.0 mmol), lactamide (715 mg, 8.0 mmol), CuI (153 mg, 0.8 mmol), DMEDA (142 mg, 1.6 mmol), and K₂CO₃ (1.11 g, 8.0 mmol) were suspended in 1,4-dioxane (20 mL) at room temperature under nitrogen atmosphere. The reaction mixture was heated to 110° C. and stirred overnight. The mixture was allowed to cool down to room temperature. The reaction mixture was diluted with DCM (25 mL) and filtrated. The filtrate was concentrated under reduced pressure. The crude residue was purified by silica gel chromatography (MeOH/DCM 0-5%) to afford N-(5-(3,5-difluorophenyl)-6,7-dihydro-5H-pyrrolo[1,2-a]imidazol-2-yl)-2-hydroxypropanamide (1.00 g, 81% yield) as a light yellow oil.

LCMS (ESI) [M+H]⁺: 308.

### Step 5: 1-((5-(3,5-difluorophenyl)-6,7-dihydro-5H-pyrrolo[1,2-a]imidazol-2-yl)amino)-1-oxopropan-2-yl 2-nitrobenzenesulfonate

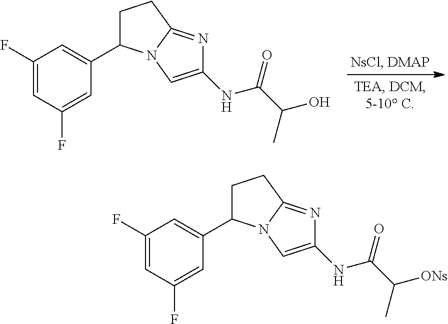

Et₃N (109 mg, 1.1 mmol) and DMAP (4 mg, 40 μmol) were added to a mixture of N-(5-(3,5-difluorophenyl)-6,7-dihydro-5H-pyrrolo[1,2-a]imidazol-2-yl)-2-hydroxypropanamide (110 mg, 0.4 mmol) in DCM (5 mL) at 0° C. under nitrogen atmosphere. After 5 min, 2-nitrobenzenesulfonyl chloride (87 mg, 0.4 mmol) was added at 0° C. The reaction mixture was stirred for 1 h at 5-10° C. After completion, the reaction mixture was directly purified through silica gel chromatography (MeOH/DCM 0-5%) to afford 1-((5-(3,5-difluorophenyl)-6,7-dihydro-5H-pyrrolo[1,2-a]imidazol-2-yl)amino)-1-oxopropan-2-yl 2-nitrobenzenesulfonate (108 mg, 61% yield) as a yellow oil.

LCMS (ESI) [M+H]⁺: 493.

### Step 6: 4-((3S)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

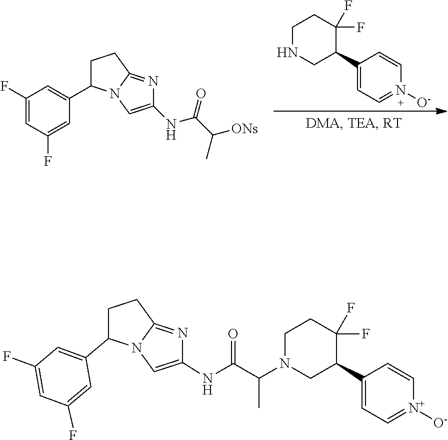

A mixture of 1-((5-(3,5-difluorophenyl)-6,7-dihydro-5H-pyrrolo[1,2-a]imidazol-2-yl)amino)-1-oxopropan-2-yl 2-nitrobenzenesulfonate (108 mg, 0.2 mmol), (S)-4-(4,4-difluoropiperidin-3-yl)pyridine 1-oxide (Intermediate B, 56 mg, 0.3 mmol) and TEA (67 mg, 0.7 mmol) in N,N-dimethylacetamide (5 mL) was stirred at room temperature for 12 h under nitrogen atmosphere. The resulting mixture was concentrated under vacuum. The crude residue was purified by reverse-phase flash chromatography (column, C18 silica gel; mobile phase, MeCN in Water (10 mmol/L NH₄HCO₃), 0% to 100% gradient in 20 min; detector, UV 254 nm and UV 220 nm) to afford 4-((3S)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (70 mg, 63% yield) as a white solid.

LCMS (ESI) [M+H]⁺: 504.

### Step 7: 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide and 4-((S)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide and 4-((S)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide and 4-((S)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

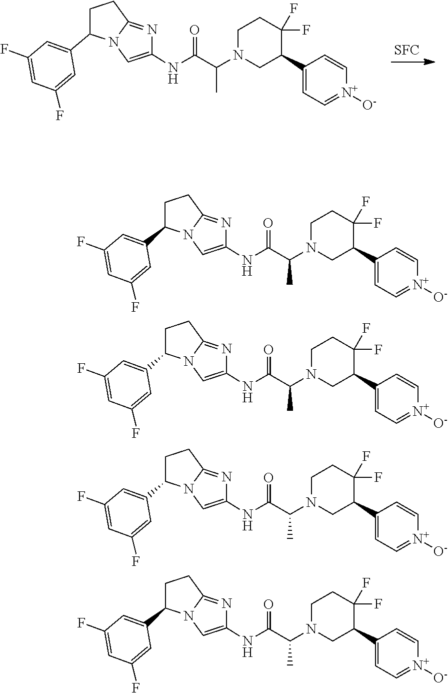 [SMILES: CC(C(=O)Nc1cn2c(n1)CCC2c1cc(F)cc(F)c1)N1CCC(F)(F)[C@@H](c2cc[n+]([O-])cc2)C1 ; "SFC" ; C[C@@H](C(=O)Nc1cn2c(n1)CC[C@@H]2c1cc(F)cc(F)c1)N1CCC(F)(F)[C@@H](c2cc[n+]([O-])cc2)C1 ; C[C@@H](C(=O)Nc1cn2c(n1)CC[C@H]2c1cc(F)cc(F)c1)N1CCC(F)(F)[C@@H](c2cc[n+]([O-])cc2)C1 ; C[C@H](C(=O)Nc1cn2c(n1)CC[C@H]2c1cc(F)cc(F)c1)N1CCC(F)(F)[C@@H](c2cc[n+]([O-])cc2)C1 ; C[C@H](C(=O)Nc1cn2c(n1)CC[C@@H]2c1cc(F)cc(F)c1)N1CCC(F)(F)[C@@H](c2cc[n+]([O-])cc2)C1]

4-((3S)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (70 mg) was purified by SFC (Column: CHIRAL ART Cellulose-SZ, 3*25 cm, 5 μm; Mobile Phase A: Hex (10 mM NH₃-MeOH), Mobile Phase B: EtOH; Flow rate: 40 mL/min; Gradient: isocratic 50% B; Wave Length: 200/273 nm; to afford 4-((S)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (Compound 486) (RT1: 17 min; Method V, 1.62 min, Peak 1, 3.0 mg, 100% e.e.) as a white solid, 4-((S)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (Compound 488) (RT2: 25 min; Method V, 2.99 min, peak 2, 18.0 mg, 100% e.e.) as a white solid and a mixture (Compound 487 and Compound 489) (RT3: 28 min, 35 mg). The mixture (Compound 487 and Compound 489) was further separated by Prep-Chiral HPLC (Column: CHIRALPAK IE, 3*25 cm, 5 μm; Mobile Phase A: MTBE:DCM=1:1 (10 mM NH₃), Mobile Phase B: MEOH; Flow rate: 40 mL/min; Gradient: isocratic 50% B; Wave Length: 302/232 nm) to afford 4-((S)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (Compound 487) (RT1: 10.5 min; Method W, 3.59 min, peak 1, 4.7 mg, 100% e.e.) as a white solid and 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide (Compound 489) (RT2: 13 min; Method W, 4.17 min, peak 2, 17.4 mg, 100% e.e.) as a white solid.

TABLE 26

| Compound# | Characterization Data |
|---|---|
| 486 | LCMS [M + H]⁺: 504.3 |
| | ¹H NMR (400 MHz, DMSO-d₆) δ 10.19 (s, 1H), 8.16 (d, J = 6.8 Hz, 2H), 7.37 (d, J = 6.6 Hz, |
| | 2H), 7.24-7.18 (m, 1H), 6.99 (s, 1H), 6.96-6.87 (m, 2H), 5.40-5.36 (m, 1H), 3.56-3.39 |
| | (m, 2H), 3.09-2.86 (m, 4H), 2.83-2.73 (m, 1H), 2.70-2.62 (m, 2H), 2.41-2.36 (m, 1H), |
| | 2.15-1.98 (m, 2H), 1.17 (d, J = 6.8 Hz, 3H) |
| 487 | LCMS [M + H]⁺: 504.2 |
| | ¹H NMR (400 MHz, DMSO-d₆) δ 10.18 (s, 1H), 8.16 (d, J = 7.2 Hz, 2H), 7.36 (d, J = 7.2 Hz, |
| | 2H), 7.23-7.17 (m, 1H), 6.99 (s, 1H), 6.96-6.88 (m, 2H), 5.42-5.36 (m, 1H), 3.59-3.37 |
| | (m, 2H), 3.06-2.64 (m, 6H), 2.54-2.46 (m, 1H), 2.39-2.33 (m, 1H), 2.19-1.96 (m, 2H), |
| | 1.16 (d, J = 6.8 Hz, 3H) |
| 488 | LCMS [M + H]⁺: 504.2 |
| | ¹H NMR (400 MHz, DMSO-d₆) δ 10.13 (s, 1H), 8.16 (d, J = 7.2 Hz, 2H), 7.38 (d, J = 7.2 Hz, |
| | 2H), 7.24-7.19 (m, 1H), 7.00-6.90 (m, 3H), 5.37 (dd, J = 7.8, 5.3 Hz, 1H), 3.57-3.40 (m, |
| | 2H), 3.01-2.88 (m, 4H), 2.83-2.59 (m, 3H), 2.44-2.31 (m, 1H), 2.18-1.93 (m, 2H), 1.18 |
| | (d, J = 7.2 Hz, 3H) |
| 489 | LCMS [M + H]⁺: 504.2 |
| | ¹H NMR (400 MHz, DMSO-d₆) δ 10.12 (s, 1H), 8.16 (d, J = 6.8 Hz, 2H), 7.37 (d, J = 6.8 Hz, |
| | 2H), 7.25-7.20 (m, 1H), 6.99-6.90 (m, 3H), 5.38 (dd, J = 7.8, 5.3 Hz, 1H), 3.60-3.39 (m, |
| | 2H), 3.04-2.72 (m, 6H), 2.39-2.33 (m, 2H), 2.19-1.92 (m, 2H), 1.16 (d, J = 6.8 Hz, 3H) |

### Step 8: Compound 489 Absolute Crystal Structure Determination

The Compound 489 crystalline sample for MicroED test was obtained by vapor diffusion in ACN/DIPE. The XRPD pattern is shown in FIG. 1A, and the corresponding characteristic peaks are listed in the Table in FIG. 1B. The crystals obtained were rod-like, and the particle size of crystals was typically less than 20 μm.

The absolute crystal structure of Compound 489 shown below and in FIG. 2A was successfully determined by the MicroED method. The crystal belongs to the hexagonal system and P6₅ (No. 170) space group. The unit-cell parameters are a=37.2(3) Å, b=37.2(3) Å, c=6.67(5) Å, α=90°, β=90°, γ=120°, with a volume of V=8013(130) Å³. The Z′ for the system is 2 and the asymmetric unit of the crystal is comprised of 2 Compound 489 molecules and some highly disordered solvent molecules. The crystal system of Compound 489 is hexagonal, with a space group of P6₅ (No. 170) and a Z′ of 2. The molecular weight is 548.54 g·mol⁻¹. The crystallographic data and refinement parameters are listed in the Table in FIG. 2B.

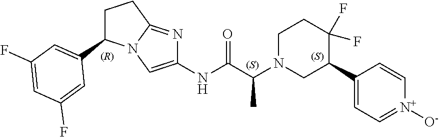

### Example 53b: Prophetic Synthetic Method for 4-((3R)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

### Step 1: 4-((3R)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

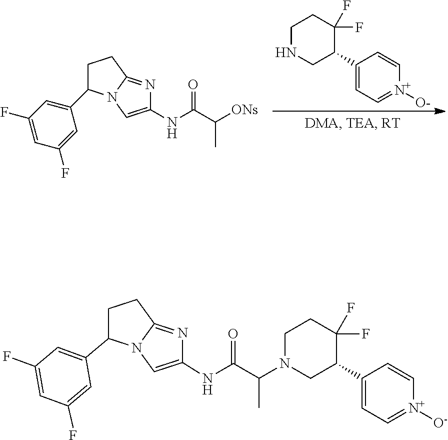

A mixture of 1-((5-(3,5-difluorophenyl)-6,7-dihydro-5H-pyrrolo[1,2-a]imidazol-2-yl)amino)-1-oxopropan-2-yl 2-nitrobenzenesulfonate (1 equiv), (R)-4-(4,4-difluoropiperidin-3-yl)pyridine 1-oxide (Intermediate C, 1.5 equiv) and TEA (3.5 equiv) in N,N-dimethylacetamide (0.01-0.1 M) is stirred at room temperature for 12-24 h under nitrogen atmosphere. The resulting mixture is concentrated under vacuum. The resulting crude residue is purified by reverse-phase flash chromatography to afford 4-((3R)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide.

### Step 2: 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, 4-((R)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, 4-((R)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, and 4-((R)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide

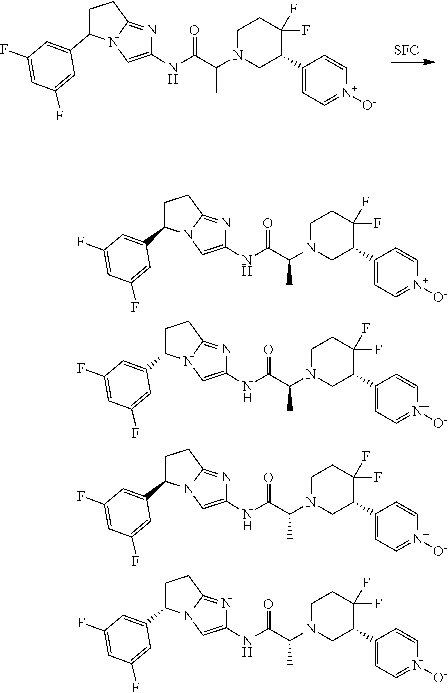 [SMILES: CC(C(=O)Nc1cn2c(n1)CCC2c1cc(F)cc(F)c1)N1CCC(F)(F)[C@H](c2cc[n+]([O-])cc2)C1 ; "SFC" ; C[C@@H](C(=O)Nc1cn2c(n1)CC[C@@H]2c1cc(F)cc(F)c1)N1CCC(F)(F)[C@H](c2cc[n+]([O-])cc2)C1 ; C[C@@H](C(=O)Nc1cn2c(n1)CC[C@H]2c1cc(F)cc(F)c1)N1CCC(F)(F)[C@H](c2cc[n+]([O-])cc2)C1 ; C[C@H](C(=O)Nc1cn2c(n1)CC[C@@H]2c1cc(F)cc(F)c1)N1CCC(F)(F)[C@H](c2cc[n+]([O-])cc2)C1 ; C[C@H](C(=O)Nc1cn2c(n1)CC[C@H]2c1cc(F)cc(F)c1)N1CCC(F)(F)[C@H](c2cc[n+]([O-])cc2)C1]

4-((3R)-1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide is purified by SFC to afford 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, 4-((R)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, 4-((R)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, and 4-((R)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide.

### Example 54: (2S)-2-(4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide

### Step 1: benzyl 3-(5-(1,3-dioxolan-2-yl)-6-methoxypyridin-3-yl)-4,4-difluoropiperidine-1-carboxylate

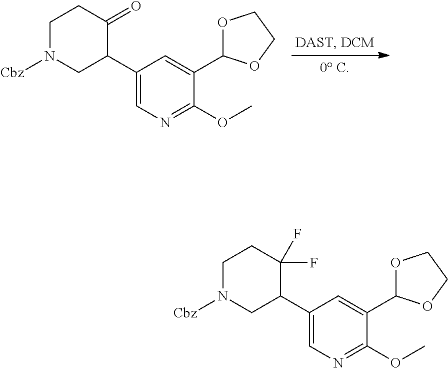 [SMILES: COc1ncc(C2CN(C(=O)OCc3ccccc3)CCC2=O)cc1C1OCCO1 ; COc1ncc(C2CN(C(=O)OCc3ccccc3)CCC2(F)F)cc1C1OCCO1]

DAST (7.04 g, 43.6 mmol) was added dropwise, over 5 min, to a solution of benzyl 3-(5-(1,3-dioxolan-2-yl)-6-methoxypyridin-3-yl)-4-oxopiperidine-1-carboxylate (6.00 g, 14.6 mmol) in DCM (60 mL) at 0° C. The resulting mixture was stirred at room temperature for additional 2 h. The reaction was quenched by the addition of sat. sodium bicarbonate (aq.) at 0° C. The aqueous layer was extracted with EtOAc (3×100 mL). The combined organic layers were washed with brine, dried over anhydrous Na₂SO₄, filtered, and concentrated under reduced pressure. The crude residue was purified by silica gel column chromatography, eluted with PE/EA (1:1) to afford benzyl 3-(5-(1,3-dioxolan-2-yl)-6-methoxypyridin-3-yl)-4,4-difluoropiperidine-1-carboxylate (4.00 g, 63% yield) as a yellow oil.

LCMS (ESI) [M+H]⁺: 435.

### Step 2: benzyl 4,4-difluoro-3-(5-formyl-6-methoxypyridin-3-yl)piperidine-1-carboxylate

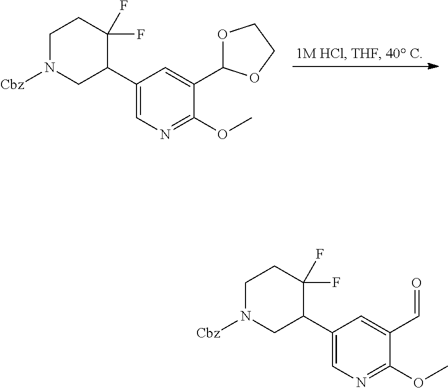 [SMILES: COc1ncc(C2CN(C(=O)OCc3ccccc3)CCC2(F)F)cc1C1OCCO1 ; COc1ncc(C2CN(C(=O)OCc3ccccc3)CCC2(F)F)cc1C=O]

Benzyl 3-(5-(1,3-dioxolan-2-yl)-6-methoxypyridin-3-yl)-4,4-difluoropiperidine-1-carboxylate (4.00 g, 9.2 mmol) in 1M HCl in H₂O (20 mL) and THF (20 mL) was stirred at 40° C. for 2 h under nitrogen atmosphere. The mixture was allowed to cool down to room temperature. The aqueous layer was extracted with CH₂Cl₂ (3×100 mL). The combined organic layers were washed brine, dried over anhydrous Na₂SO₄, filtered and concentrated under reduced pressure. The crude residue was purified by silica gel column chromatography, eluted with PE/EA (1:1) to afford benzyl 4,4-difluoro-3-(5-formyl-6-methoxypyridin-3-yl)piperidine-1-carboxylate (3.00 g, 84% yield) as a light yellow oil.

LCMS (ESI) [M+H]⁺: 391.

### Step 3: benzyl 4,4-difluoro-3-(5-(hydroxymethyl)-6-methoxypyridin-3-yl)piperidine-1-carboxylate

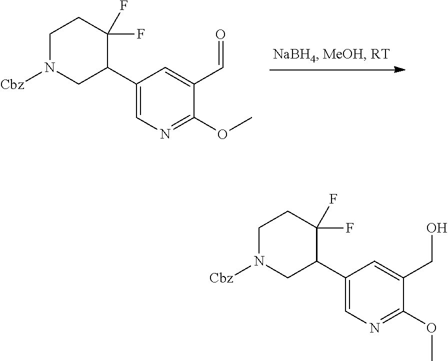 [SMILES: COc1ncc(C2CN(C(=O)OCc3ccccc3)CCC2(F)F)cc1C=O ; COc1ncc(C2CN(C(=O)OCc3ccccc3)CCC2(F)F)cc1CO]

NaBH₄ (349 mg, 9.2 mmol) was added portion-wise, over 5 min, to a solution of benzyl 4,4-difluoro-3-(5-formyl-6-methoxypyridin-3-yl)piperidine-1-carboxylate (1.20 g, 3.1 mmol) in MeOH (12 mL) at 0° C. The resulting mixture was stirred at room temperature for additional 2 h. The reaction was quenched by the addition of sat. NH₄Cl (aq.) at 0° C. The resulting mixture was concentrated under reduced pressure. The crude residue was purified by silica gel column chromatography, eluted with PE/EA (1:1) to afford benzyl 4,4-difluoro-3-(5-(hydroxymethyl)-6-methoxypyridin-3-yl)piperidine-1-carboxylate (1.00 g, 83% yield) as a colorless oil.

LCMS (ESI) [M+H]⁺: 393.

### Step 4: (5-(4,4-difluoropiperidin-3-yl)-2-methoxypyridin-3-yl)methanol

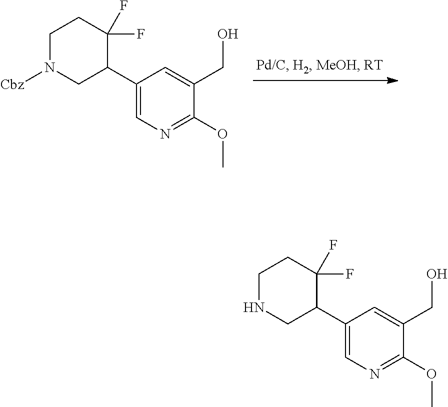 [SMILES: COc1ncc(C2CN(C(=O)OCc3ccccc3)CCC2(F)F)cc1CO ; COc1ncc(C2CNCCC2(F)F)cc1CO]

To a mixture of benzyl 4,4-difluoro-3-(5-(hydroxymethyl)-6-methoxypyridin-3-yl)piperidine-1-carboxylate (1.00 g, 2.5 mmol) in MeOH (12 mL) was added Pd/C (81 mg, 0.8 mmol) under hydrogen atmosphere. Upon completion, the resulting mixture was filtered through celite. The filter cake was washed with MeOH (3×20 mL). The filtrate was concentrated under reduced pressure. The crude residue was purified by reversed-phase flash chromatography (column, C18 silica gel; mobile phase, MeCN in water (0.1% formic acid), 0% to 100% gradient in 20 min; detector, UV 254 nm and UV 220 nm) to give (5-(4,4-difluoropiperidin-3-yl)-2-methoxypyridin-3-yl)methanol (600 mg, 91% yield) as a white solid.

LCMS (ESI) [M+H]⁺: 259.

### Step 5: (2S)-2-(4,4-difluoro-3-(5-(hydroxymethyl)-6-methoxypyridin-3-yl)piperidin-1-yl)-N-(1-(3,5-difluorobenzyl)-2-(difluoromethyl)-1H-imidazol-4-yl)propanamide

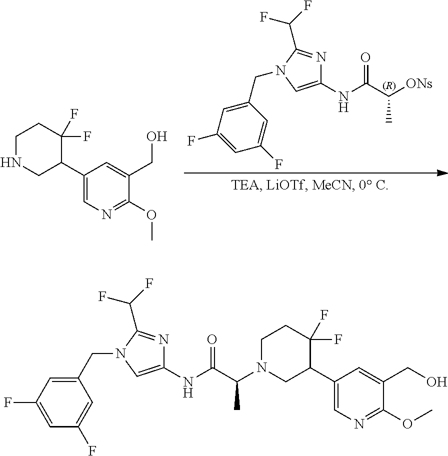

A solution of (5-(4,4-difluoropiperidin-3-yl)-2-methoxypyridin-3-yl)methanol (200 mg, 770.0 μmol), (R)-1-((1-(3,5-difluorobenzyl)-2-(difluoromethyl)-1H-imidazol-4-yl)amino)-1-oxopropan-2-yl 2-nitrobenzenesulfonate (490 mg, 930.0 μmol), Et₃N (196 mg, 1.9 mmol) and lithium trifluoromethanesulfonate (302 mg, 1.9 mmol) in ACN (10 mL) was stirred at 0° C. for overnight under nitrogen atmosphere. The resulting mixture was concentrated under reduced pressure. The crude residue was purified by reversed-phase flash chromatography (column, C18 silica gel; mobile phase, MeCN in water (0.1% formic acid), 0% to 100% gradient in 20 min; detector, UV 254 nm) to give (2S)-2-(4,4-difluoro-3-(5-(hydroxymethyl)-6-methoxypyridin-3-yl)piperidin-1-yl)-N-(1-(3,5-difluorobenzyl)-2-(difluoromethyl)-1H-imidazol-4-yl)propanamide (200 mg, 45% yield) as a light yellow solid.

LCMS (ESI) [M+H]⁺: 572.

### Step 6: (2S)-2-(4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide

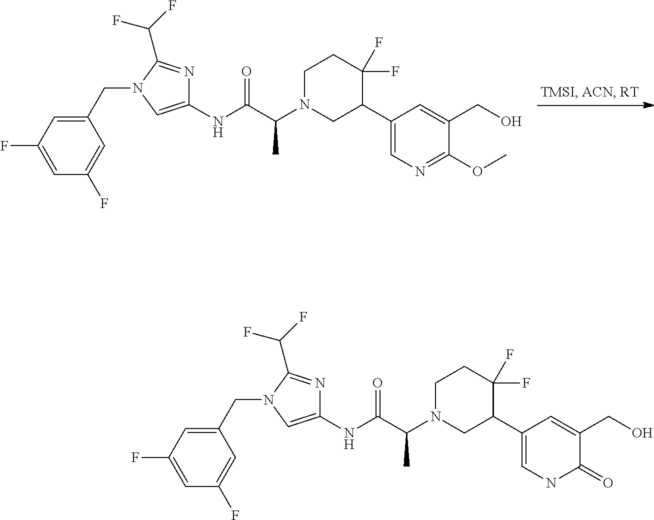 [SMILES: COc1ncc(C2CN([C@@H](C)C(=O)Nc3cn(Cc4cc(F)cc(F)c4)c(C(F)F)n3)CCC2(F)F)cc1CO ; C[C@@H](C(=O)Nc1cn(Cc2cc(F)cc(F)c2)c(C(F)F)n1)N1CCC(F)(F)C(c2c[nH]c(=O)c(CO)c2)C1]

Iodotrimethylsilane (350 mg, 1.8 mmol) was added dropwise over 10 min to a solution of (2S)-2-(4,4-difluoro-3-(5-(hydroxymethyl)-6-methoxypyridin-3-yl)piperidin-1-yl)-N-(1-(3,5-difluorobenzyl)-2-(difluoromethyl)-1H-imidazol-4-yl)propanamide (200 mg, 350.0 μmol) in ACN (6 mL) at 0° C. The resulting mixture was stirred at room temperature for additional 6 h and then was concentrated under reduced pressure. The crude residue was purified by reversed-phase flash chromatography (column, C18 silica gel; mobile phase, MeCN in water (0.1% formic acid), 0% to 100% gradient in 20 min; detector, UV 254 nm) to give (2S)-2-(4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide (60 mg, 31% yield) as a brown solid.

LCMS (ESI) [M+H]⁺: 558.

### Step 7: (S)-2-((S)-4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide and (S)-2-((R)-4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide

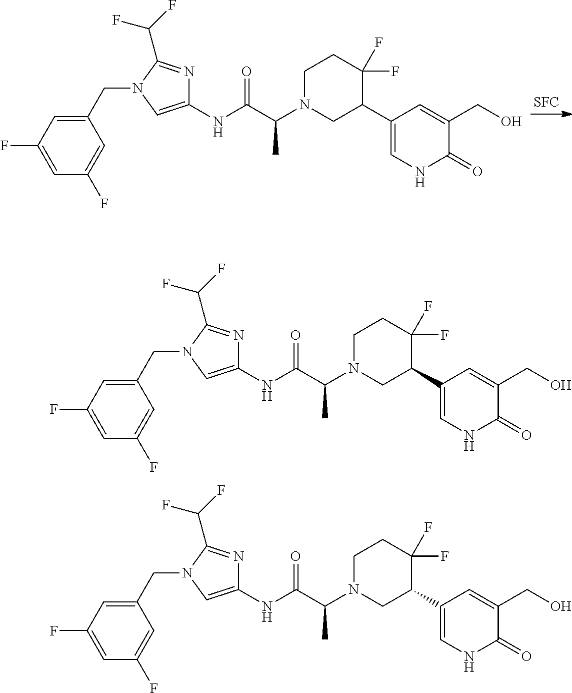

(2S)-2-(4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide (50 mg) was purified by Prep-Chiral HPLC (Column: Lux 5 μm Cellulose-2, 30*250 mm, 5.0 μm; Mobile Phase A: Hex (10 mM NH₃-MeOH), Mobile Phase B: EtOH; Flow rate: 40 mL/min; Gradient: isocratic 50% B; Wave Length: 234/300 nm) to afford (S)-2-((S)-4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide (Compound 490) (RT1: 10.3 min; Method X, 1.79 min, peak 1, 15.9 mg, 100% e.e.) as a white solid and second peak (S)-2-((R)-4,4-difluoro-3-(5-(hydroxymethyl)-6-oxo-1,6-dihydropyridin-3-yl)piperidin-1-yl)-N-(1-(3,5-difluorobenzyl)-2-(difluoromethyl)-1H-imidazol-4-yl)propanamide (Compound 491) (RT2: 13.4 min; Method X, 2.45 min, peak 2, 16.1 mg, 98% e.e.) as a white solid.

TABLE 27

| Compound# | Characterization Data |
|---|---|
| 490 | LCMS [M + H]⁺: 558.2 |
| | ¹H NMR (400 MHz, DMSO-d₆) δ 11.56 (s, 1H), 10.46 (s, 1H), 7.56 (s, 1H), 7.42 (s, 1H), 7.34- |
| | 7.03 (m, 3H), 6.97-6.95 (m, 2H), 5.36 (s, 2H), 5.13-5.06 (m, 1H), 4.28 (d, J = 5.0 Hz, 2H), |
| | 3.57-3.53 (m, 1H), 3.27-3.06 (m, 1H), 2.97-2.69 (m, 3H), 2.49-2.42 (m, 1H), 2.19- |
| | 1.96 (m, 2H), 1.18 (d, J = 6.8 Hz, 3H) |
| 491 | LCMS [M + H]⁺: 558.2 |
| | ¹H NMR (400 MHz, DMSO-d₆) δ 11.53 (s, 1H), 10.46 (s, 1H), 7.53 (s, 1H), 7.40 (s, 1H), 7.34- |
| | 7.03 (m, 3H), 6.97-6.95 (m, 2H), 5.36 (s, 2H), 5.07-5.04 (m, 1H), 4.26 (d, J = 5.0 Hz, 2H), |
| | 3.52-3.47 (m, 1H), 3.22-3.16 (m, 2H), 2.99-2.76 (m, 2H), 2.65-2.60 (m, 2H), 2.17-1.96 |
| | (m, 2H), 1.19 (d, J = 6.8 Hz, 3H) |

### Example 55: (S)—N-(1-(3,5-difluorobenzyl)-2-(difluoromethyl)-1H-imidazol-4-yl)-2-(4,4-difluoropiperidin-1-yl)propanamide

### Step 1: (S)—N-(1-(3,5-difluorobenzyl)-2-(difluoromethyl)-1H-imidazol-4-yl)-2-(4,4-difluoropiperidin-1-yl)propanamide

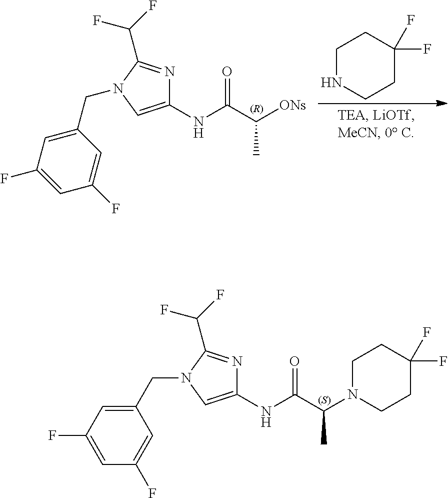

A mixture of (R)-1-((1-(3,5-difluorobenzyl)-2-(difluoromethyl)-1H-imidazol-4-yl)amino)-1-oxopropan-2-yl 2-nitrobenzenesulfonate (50 mg, 0.1 mmol), 4,4-difluoropiperidine (14 mg, 0.1 mmol), TEA (29 mg, 0.3 mmol) and lithium trifluoromethanesulfonate (48 mg, 0.3 mmol) in acetonitrile (2 mL) was stirred for 12 hours at 0° C. The resulting mixture was poured into water (5 mL) and extracted with DCM (3×10 mL). The combined organic layers were dried over MgSO₄, filtered and concentrated. The crude residue was purified by Prep-HPLC (Column: XSelect CSH Fluoro Phenyl 30*150 mm, 5 μm; Mobile Phase A: Water (0.1% FA), Mobile Phase B: ACN; Flow rate: 60 mL/min; Gradient: 18% B to 38% B in 10 min; Wave Length: 254 nm/220 nm) to afford (S)—N-(1-(3,5-difluorobenzyl)-2-(difluoromethyl)-1H-imidazol-4-yl)-2-(4,4-difluoropiperidin-1-yl)propanamide (Compound 492) (RT1: 6.2 min; 19.9 mg, 47.3% yield) as a white solid.

TABLE 28

| Compound# | Characterization Data |
|---|---|
| 492 | LCMS [M + H]⁺: 435.1 |
| | ¹H NMR (400 MHz, DMSO-d₆) δ 10.36 (s, 1H), |
| | 7.52 (s, 1H), 7.27-7.15 (m, 2H), 6.95 (d, J = 6.0 |
| | Hz, 2H), 5.36 (s, 2H), 3.52-3.40 (m, 1H), 2.68-2.56 (m, |
| | 4H), 2.09-1.86 (m, 4H), 1.15 (d, J = 6.8 Hz, 3H) |

### Example 56: (2S)-2-(4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide

### Step 1: (2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide

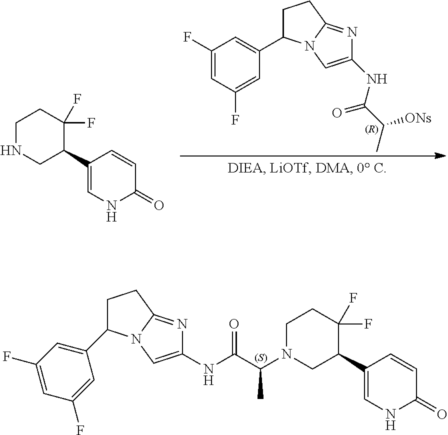

To a mixture of (2R)-1-((5-(3,5-difluorophenyl)-6,7-dihydro-5H-pyrrolo[1,2-a]imidazol-2-yl)amino)-1-oxopropan-2-yl 2-nitrobenzenesulfonate (200 mg, 0.4 mmol) and (S)-5-(4,4-difluoropiperidin-3-yl)pyridin-2(1H)-one (87 mg, 0.4 mmol) in N,N-dimethylacetamide (6 mL) was added lithium trifluoromethanesulfonate (127 mg, 0.8 mmol) and DIEA (105 mg, 0.8 mmol) dropwise at 0° C. The resulting mixture was stirred at 0° C. for 20 h under nitrogen atmosphere. The resulting mixture was concentrated under reduced pressure and the crude residue was purified by reversed-phase flash chromatography (column, C18 silica gel; mobile phase, MeCN in water (0.1% NH₄HCO₃), 0% to 100% gradient in 20 min; detector, UV 220 nm) to give (2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide (50 mg, 24% yield) as a yellow solid.

LCMS (ESI) [M+H]⁺: 504.

### Step 2: (S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide

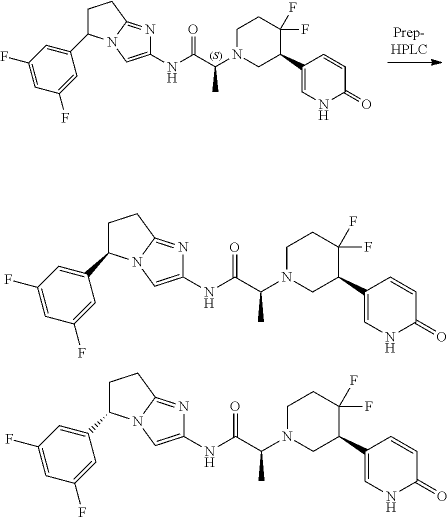

(2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide (50 mg) was purified by Prep-HPLC (Column: Xselect CSH Prep C18, 30*150 mm 5 μm; Mobile Phase A: Water (0.1% FA), Mobile Phase B: ACN; Flow rate: 60 mL/min; Gradient: 5% B to 5% B in 1.5 min, 5% B to 12% B in 2 min, 12% to 26% B in 15 min; Wave Length: 254 nm/220 nm) to afford (S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 493) (RT1: 9.62 min; Method Y, 1.77 min, peak 2, 5.0 mg, 100% e.e.) as an off white solid and (S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide (Compound 494) (RT2: 10.45 min; Method Y, 1.37 min, peak 1, 4.5 mg, 100% e.e.) as an off white solid.

TABLE 29

| Compound# | Characterization Data |
|---|---|
| 493 | LCMS 504.2 |
| | ¹H NMR (300 MHz, DMSO-d₆) δ 11.55 (s, 1H), 10.16 (s, 1H), 7.42-7.35 (m, 1H), 7.29- |
| | 7.17 (m, 2H), 6.99 (s, 1H), 6.95-6.89 (m, 2H), 6.29 (d, J = 9.4 Hz, 1H), 5.50-5.29 (m, 1H), |
| | 3.58-3.51 (m, 1H), 3.06 (m, 7H), 2.46-2.29 (m, 2H), 2.18-1.96 (m, 2H), 1.15 (d, J = 6.8 |
| | Hz, 3H) |
| 494 | LCMS 504.2 |
| | ¹H NMR (300 MHz, DMSO-d₆) δ 11.55 (s, 1H), 10.09 (s, 1H), 7.41 (d, J = 2.5 Hz, 1H), 7.24 |
| | (d, J = 17.5, 7.9 Hz, 2H), 7.09-6.92 (m, 3H), 6.28 (d, J = 9.4 Hz, 1H), 5.53-5.27 (m, 1H), |
| | 3.56-3.50 (m, 1H), 3.05-2.61 (m, 7H), 2.46-2.38 (m, 2H), 2.08-1.96 (m, 2H), 1.16 (d, |
| | J = 6.8 Hz, 3H) |

### Step 3: (2S)-2-((R)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide

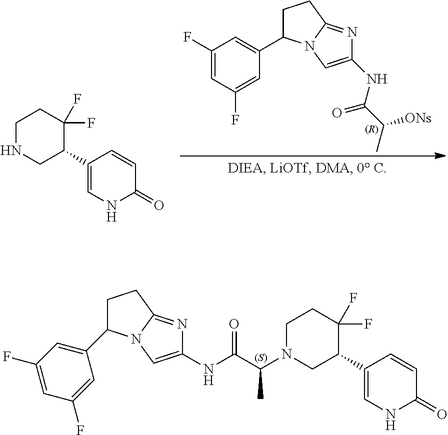

To a mixture of (2R)-1-((5-(3,5-difluorophenyl)-6,7-dihydro-5H-pyrrolo[1,2-a]imidazol-2-yl)amino)-1-oxopropan-2-yl 2-nitrobenzenesulfonate (200 mg, 0.4 mmol) and (R)-5-(4,4-difluoropiperidin-3-yl)pyridin-2(1H)-one (87 mg, 0.4 mmol) in N,N-dimethylacetamide (5 mL) was added lithium trifluoromethanesulfonate (127 mg, 0.8 mmol) and DIEA (105 mg, 0.8 mmol) dropwise at 0° C. under nitrogen atmosphere. The resulting mixture was stirred at 0° C. for 20 h. The mixture was concentrated under reduced pressure and the crude residue was purified by reversed-phase flash chromatography (column, C18 silica gel; mobile phase, MeCN in Water (0.1% NH₄HCO₃), 0% to 100% gradient in 20 min; detector, UV 220 nm) to give (2S)-2-((R)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide (80 mg, 39% yield) as a yellow solid.

LCMS (ESI) [M+H]⁺: 504.

### Step 4: (S)-2-((R)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide and (S)-2-((R)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide

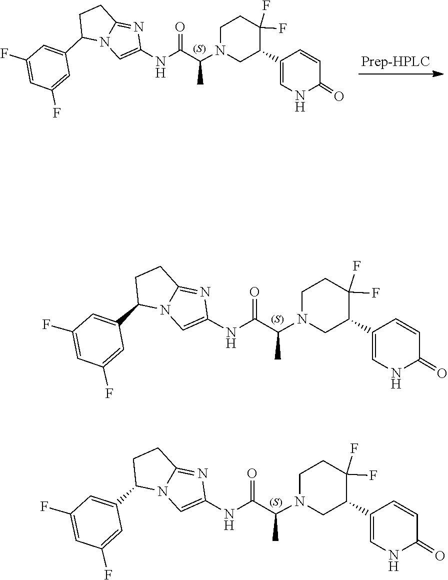

(2S)-2-((R)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide (80 mg) was purified by Prep-HPLC (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 26% B to 36% B in 15 min; Wave Length: 254 nm/220 nm) to afford (S)-2-((R)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 495) (RT1: 12.88 min; Method Z, 2.94 min, peak 1, 7.8 mg, 99.1% e.e.) as a white solid and (S)-2-((R)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide (Compound 496) (RT2: 15.38 min; Method Z, 3.56 min, peak 2, 4.2 mg, 100% e.e.) as a white solid.

TABLE 30

| Compound# | Characterization Data |
|---|---|
| 495 | LCMS 504.2 |
| | ¹H NMR (300 MHz, DMSO-d₆) δ 11.55 (s, 1H), 10.16 (s, 1H), 7.59-7.48 (m, 1H), 7.36- |
| | 7.28 (m, 1H), 7.27-7.17 (m, 1H), 7.09-6.93 (m, 3H), 6.29 (d, J = 9.4 Hz, 1H), 5.50-5.31 |
| | (m, 1H), 3.09-2.99 (m, 3H), 2.92-2.87 (m, 3H), 2.82-2.78 (m, 1H), 2.62-2.57 (m, 1H), |
| | 2.46-2.29 (m, 2H), 2.16-1.96 (m, 2H), 1.15 (d, J = 6.8 Hz, 3H). |
| 496 | LCMS 504.2 |
| | ¹H NMR (300 MHz, DMSO-d₆) δ 11.55 (s, 1H), 10.16 (s, 1H), 7.40-7.30 (m, 1H), 7.30- |
| | 7.21 (m, 1H), 7.20-7.17 (m, 1H), 7.01 (s, 1H), 6.99-6.93 (m, 2H), 6.29 (d, J = 9.4 Hz, 1H), |
| | 5.50-5.31 (m, 1H), 3.06 (s, 2H), 2.92-2.87 (m, 3H), 2.90-2.86 (m, 1H), 2.80-2.76 (m, |
| | 1H), 2.60-2.56 (m, 1H), 2.46-2.29 (m, 2H), 2.16-2.03 (s, 2H), 1.15 (d, J = 6.8 Hz, 3H) |

### Example 57: N-(5-(3,5-difluorophenyl)-6,7-dihydro-5H-pyrrolo[1,2-a]imidazol-2-yl)-2-(piperidin-1-yl)propanamide

### Step 1: N-(5-(3,5-difluorophenyl)-6,7-dihydro-5H-pyrrolo[1,2-a]imidazol-2-yl)-2-(piperidin-1-yl)propanamide

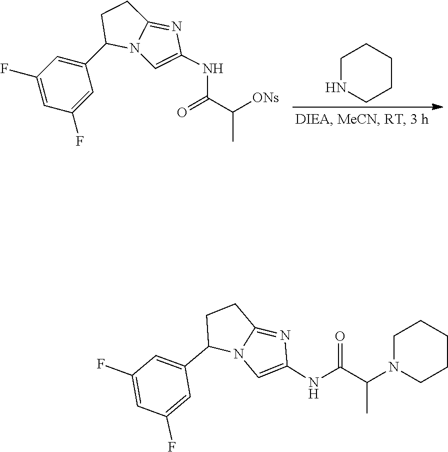

DIEA (157 mg, 1.2 mmol) was added dropwise to a mixture of piperidine (42 mg, 487 μmol) and 1-((5-(3,5-difluorophenyl)-6,7-dihydro-5H-pyrrolo[1,2-a]imidazol-2-yl)amino)-1-oxopropan-2-yl 2-nitrobenzenesulfonate (200 mg, 0.4 mmol) in ACN (3 mL) at room temperature under nitrogen atmosphere. The resulting mixture was stirred at room temperature for 3 h. The mixture was concentrated under reduced pressure and the crude residue was purified by reversed-phase flash chromatography (column, C18 silica gel; mobile phase, MeCN in water (10 mmol/L NH₄HCO₃), 5% to 60% gradient in 30 min; detector, UV 254 nm) to give N-(5-(3,5-difluorophenyl)-6,7-dihydro-5H-pyrrolo[1,2-a]imidazol-2-yl)-2-(piperidin-1-yl)propanamide (95 mg, 62% yield) as a light yellow semi-solid.

LCMS (ESI) [M+H]⁺: 375.

### Step 2: (S)—N—((R)-5-(3,5-difluorophenyl)-6,7-dihydro-5H-pyrrolo[1,2-a]imidazol-2-yl)-2-(piperidin-1-yl)propanamide and (R)—N—((R)-5-(3,5-difluorophenyl)-6,7-dihydro-5H-pyrrolo[1,2-a]imidazol-2-yl)-2-(piperidin-1-yl)propanamide and (R)—N—((S)-5-(3,5-difluorophenyl)-6,7-dihydro-5H-pyrrolo[1,2-a]imidazol-2-yl)-2-(piperidin-1-yl)propanamide and (S)—N—((S)-5-(3,5-difluorophenyl)-6,7-dihydro-5H-pyrrolo[1,2-a]imidazol-2-yl)-2-(piperidin-1-yl)propanamide

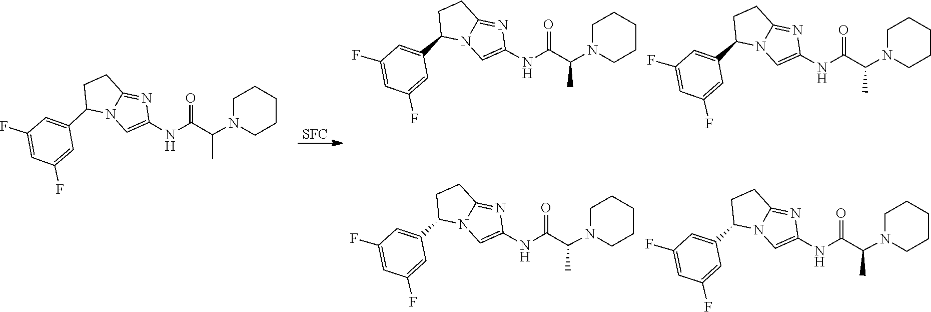

N-(5-(3,5-difluorophenyl)-6,7-dihydro-5H-pyrrolo[1,2-a]imidazol-2-yl)-2-(piperidin-1-yl)propanamide (95 mg) was purified by prep-Chiral-HPLC (Column: CHIRAL ART Cellulose-SC, 3*25 cm, 5 μm; Mobile Phase A: Hex (10 mM NH₃-MeOH), Mobile Phase B: EtOH; Flow rate: 40 mL/min; Gradient: isocratic 20% B; Wave Length: 212/242 nm) to afford (S)—N—((R)-5-(3,5-difluorophenyl)-6,7-dihydro-5H-pyrrolo[1,2-a]imidazol-2-yl)-2-(piperidin-1-yl)propanamide (Compound 497) (RT1: 7.2 min; Method AA, 1.82 min, peak 1, 6.6 mg, 97.5% e.e.) as a white solid, (R)—N—((R)-5-(3,5-difluorophenyl)-6,7-dihydro-5H-pyrrolo[1,2-a]imidazol-2-yl)-2-(piperidin-1-yl)propanamide (Compound 498) (RT2: 8.7 min; Method AA, 2.24 min, peak 2, 16.6 mg, 99.7% e.e.) as a white solid, (R)—N—((S)-5-(3,5-difluorophenyl)-6,7-dihydro-5H-pyrrolo[1,2-a]imidazol-2-yl)-2-(piperidin-1-yl)propanamide (Compound 499) (RT3: 10.7 min; Method AA, 3.05 min, peak 3, 8.4 mg, 98.7% e.e.) as a white solid and (S)—N—((S)-5-(3,5-difluorophenyl)-6,7-dihydro-5H-pyrrolo[1,2-a]imidazol-2-yl)-2-(piperidin-1-yl)propanamide (Compound 500) (RT4: 12.5 min; Method AA, 4.43 min, peak 4, 15.3 mg, 99.0% e.e.) as a white solid.

TABLE 31

| Compound# | Characterization Data |
|---|---|
| 497 | LCMS [M + H]⁺: 375.3 |
| | ¹H NMR (400 MHz, DMSO-d₆) δ 9.81 (s, 1H), 7.28-7.16 (m, 1H), 7.07-6.86 (m, 3H), 5.39 (d, |
| | J = 6.7 Hz, 1H), 3.29-3.18 (m, 1H), 3.09-2.93 (m, 2H), 2.82-2.76 (m, 1H), 2.47-2.24 (m, |
| | 5H), 1.63-1.47 (m, 4H), 1.38 (d, J = 7.4 Hz, 2H), 1.09 (d, J = 6.8 Hz, 3H) |
| 498 | LCMS [M + H]⁺: 375.2 |
| | ¹H NMR (400 MHz, DMSO-d₆) δ 9.76 (s, 1H), 7.24-7.15 (m, 1H), 6.99-6.84 (m, 3H), 5.40- |
| | 5.37 (m, 1H), 3.27-3.25 (m, 1H), 2.96-2.94 (m, 2H), 2.83-2.73 (m, 1H), 2.48-2.32 (m, 5H), |
| | 1.52-1.50 (m, 4H), 1.39-1.38 (m, 2H), 1.10 (d, J = 6.9 Hz, 3H) |
| 499 | LCMS [M + H]⁺: 375.2 |
| | ¹H NMR (400 MHz, DMSO-d₆) δ 9.80 (s, 1H), 7.27-7.14 (m, 1H), 6.96 (s, 1H), 6.93-6.87 (m, |
| | 2H), 5.46-5.30 (m, 1H), 3.29-3.20 (m, 1H), 3.04-2.84 (m, 2H), 2.83-2.72 (m, 1H), 2.47- |
| | 2.28 (m, 5H), 1.56-1.48 (m, 4H), 1.40-1.36 (m, 2H), 1.09 (d, J = 6.9 Hz, 3H) |
| 500 | LCMS [M + H]⁺: 375.2 |
| | ¹H NMR (400 MHz, DMSO-d₆) δ 9.76 (s, 1H), 7.22 -7.19(m, 1H), 6.97-6.94 (m, 3H), 5.39-5.37 |
| | (m, 1H), 3.29-3.23 (m, 1H), 2.99-2.88 (m, 2H), 2.77-2.74 (m, 1H), 2.48-2.32 (m, 5H), 1.51- |
| | 1.50 (m, 4H), 1.39-1.37 (m, 2H), 1.10 (d, J = 6.9 Hz, 3H) |

### Example 58: (2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-b][1,2,4]triazol-2-yl)propanamide

### Step 1: methyl 4-(3,5-difluorophenyl)-4-oxobutanoate

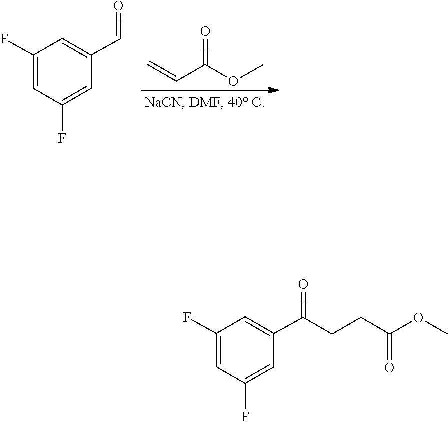

To a mixture of 3,5-difluorobenzaldehyde (10.00 g, 70.4 mmol) in DMF (80 mL) were added methyl acrylate (6.36 g, 73.9 mmol) and sodium cyanide (0.34 g, 6.9 mmol). The resulting mixture was stirred at 40° C. for 2 h under nitrogen atmosphere. After completion, the reaction mixture was cooled to room temperature and poured to a solution of saturated FeSO₄. The mixture was extracted with EtOAc (3×100 mL). The combined organic layers were dried over anhydrous Na₂SO₄ filtered and evaporated under reduced pressure. The crude residue was purified by silica gel chromatography PE/EA (9:1) to afford methyl 4-(3,5-difluorophenyl)-4-oxobutanoate (12.20 g, 76% yield) as a light yellow oil.

LCMS (ESI) [M+H]⁺: 229.

### Step 2: tert-butyl (2-(3,5-difluorophenyl)-5-oxopyrrolidin-1-yl)carbamate

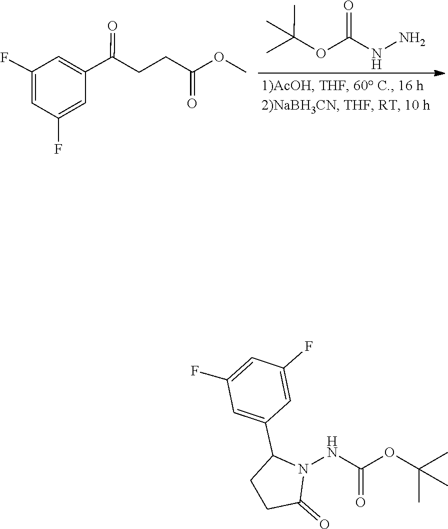

To a mixture of methyl 4-(3,5-difluorophenyl)-4-oxobutanoate (12.20 g, 53.5 mmol) in THF (100 mL) and AcOH (50 mL) was added tert-butoxycarbohydrazide (14.13 g, 106.9 mmol) at room temperature. The resulting mixture was heated to 60° C. and stirred for 16 h. The mixture was cooled to 0° C. and the sodium cyanoborohydride (10.08 g, 160.4 mmol) was added in portions at 0° C. The resulting mixture was stirred at room temperature for 10 h. After completion, the mixture was concentrated under reduced pressure to a viscous oil, then neutralized with ammonium hydroxide. The aqueous layer was extracted with EtOAc (3×200 mL). The combined organic layers were dried over anhydrous Na₂SO₄ filtered and concentrated under reduced pressure. The crude residue was purified by silica gel chromatography PE/EA (1:1) to afford tert-butyl (2-(3,5-difluorophenyl)-5-oxopyrrolidin-1-yl)carbamate (11.70 g, 70% yield) as a white solid.

LCMS (ESI) [M+H]⁺: 313.

### Step 3: 1-amino-5-(3,5-difluorophenyl)pyrrolidin-2-one

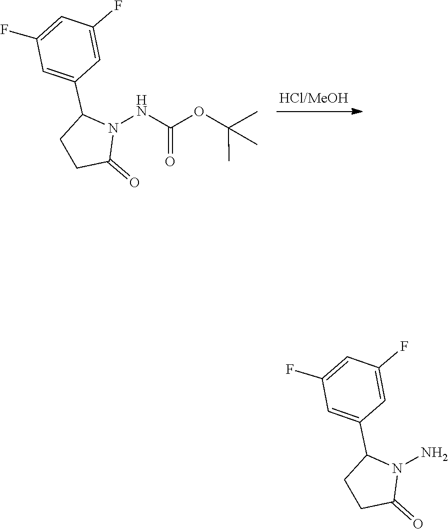 [SMILES: CC(C)(C)OC(=O)NN1C(=O)CCC1c1cc(F)cc(F)c1 ; NN1C(=O)CCC1c1cc(F)cc(F)c1]

A solution of tert-butyl (2-(3,5-difluorophenyl)-5-oxopyrrolidin-1-yl)carbamate (7.50 g, 24.0 mmol) in 4 M HCl in methanol (60 mL) was stirred for 6 h at room temperature under an ambient atmosphere. The resulting mixture was concentrated under reduced pressure to give 1-amino-5-(3,5-difluorophenyl)pyrrolidin-2-one (5.20 g, crude) as a white solid, which was used directly in the next step without further purification.

LCMS (ESI) [M+H]⁺: 213.

### Step 4: ethyl (E)-2-amino-2-((2-(3,5-difluorophenyl)-5-oxopyrrolidin-1-yl)imino)acetate

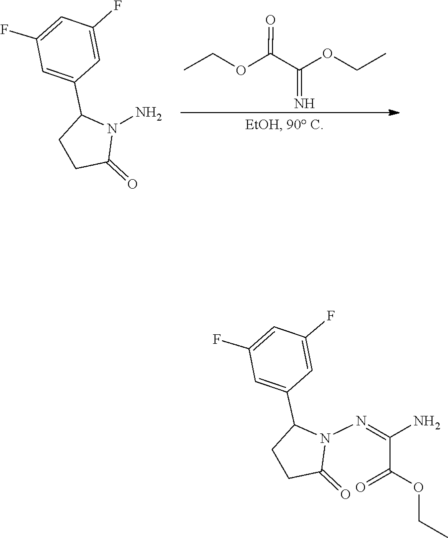

To a mixture of 1-amino-5-(3,5-difluorophenyl)pyrrolidin-2-one (5.20 g, 24.5 mmol) in EtOH (50 mL) was added ethyl 2-ethoxy-2-iminoacetate (10.67 g, 73.5 mmol) at room temperature. The resulting mixture was stirred at 90° C. for 8 h. After completion, the mixture was cooled to room temperature and concentrated under reduced pressure. The crude residue was purified by flash column chromatography DCM/MeOH (25:1) to afford ethyl (E)-2-amino-2-((2-(3,5-difluorophenyl)-5-oxopyrrolidin-1-yl)imino)acetate (6.90 g, 90% yield) as a yellow oil.

LCMS (ESI) [M+H]⁺: 312.

### Step 5: ethyl 5-(3,5-difluorophenyl)-6,7-dihydro-5H-pyrrolo[1,2-b][1,2,4]triazole-2-carboxylate

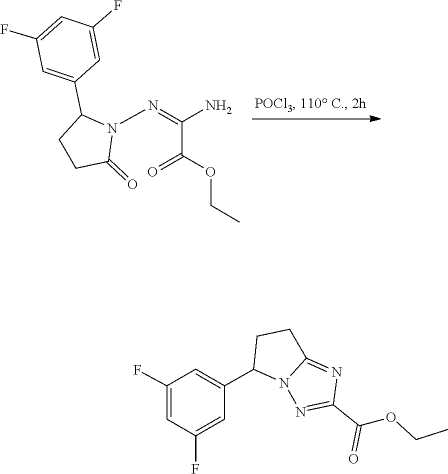 [SMILES: CCOC(=O)/C(N)=N\N1C(=O)CCC1c1cc(F)cc(F)c1 ; CCOC(=O)c1nc2n(n1)C(c1cc(F)cc(F)c1)CC2]

A mixture of ethyl (E)-2-amino-2-((2-(3,5-difluorophenyl)-5-oxopyrrolidin-1-yl)imino)acetate (6.00 g, 19.3 mmol) in POCl₃ (40 mL) was stirred at 110° C. for 3 h. After completion, the reaction mixture was cooled to room temperature, quenched with warm water and was allowed to stir for 10 min at room temperature. Ammonium hydroxide was added to the solution to neutralize pH to 6-7. The reaction mixture was extracted with EtOAc (3×80 mL). The combined organic layers were dried over anhydrous Na₂SO₄, filtered, and concentrated under reduced pressure. The crude residue was purified by silica gel chromatography DCM/MeOH (19:1) to afford ethyl 5-(3,5-difluorophenyl)-6,7-dihydro-5H-pyrrolo[1,2-b][1,2,4]triazole-2-carboxylate (3.66 g, 65% yield) as a dark green solid.

LCMS (ESI) [M+H]⁺: 294.

### Step 6: 5-(3,5-difluorophenyl)-6,7-dihydro-5H-pyrrolo[1,2-b][1,2,4]triazole-2-carboxylic acid

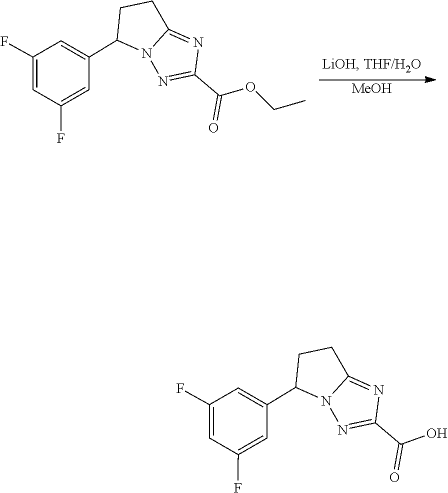 [SMILES: "MeOH" ; CCOC(=O)c1nc2n(n1)C(c1cc(F)cc(F)c1)CC2 ; O=C(O)c1nc2n(n1)C(c1cc(F)cc(F)c1)CC2]

To a mixture of ethyl 5-(3,5-difluorophenyl)-6,7-dihydro-5H-pyrrolo[1,2-b][1,2,4]triazole-2-carboxylate (3.60 g, 12.3 mmol) in THF (12 mL) were added MeOH (12 mL), H₂O (12 mL) and LiOH (1.47 g, 61.4 mmol). The resulting mixture was stirred at room temperature for 5 h. After completion, the reaction mixture was acidified with 1 M HCl to pH 3-4. The aqueous layer was extracted with EtOAc (3×50 mL). The combined organic layer were dried over anhydrous Na₂SO₄, filtered, and concentrated under reduced pressure to afford the 5-(3,5-difluorophenyl)-6,7-dihydro-5H-pyrrolo[1,2-b][1,2,4]triazole-2-carboxylic acid (2.00 g, crude) as an orange solid, which was used directly in the next step without further purification.

LCMS (ESI) [M+H]⁺: 266.

### Step 7: tert-butyl (5-(3,5-difluorophenyl)-6,7-dihydro-5H-pyrrolo[1,2-b][1,2,4]triazol-2-yl)carbamate

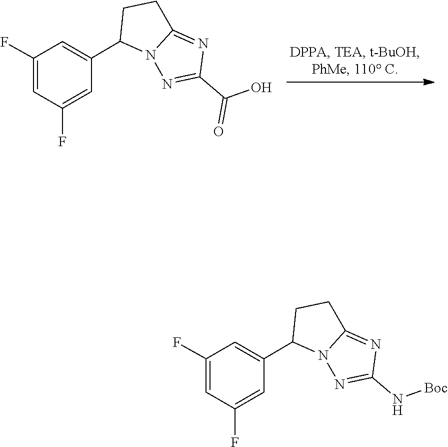 [SMILES: O=C(O)c1nc2n(n1)C(c1cc(F)cc(F)c1)CC2 ; CC(C)(C)OC(=O)Nc1nc2n(n1)C(c1cc(F)cc(F)c1)CC2]

To a mixture of 5-(3,5-difluorophenyl)-6,7-dihydro-5H-pyrrolo[1,2-b][1,2,4]triazole-2-carboxylic acid (1.95 g, 7.4 mmol) and TEA (2.23 g, 22.0 mmol) in toluene (50 mL) was added t-BuOH (10 mL) and DPPA (3.57 g, 9.6 mmol) at room temperature under nitrogen atmosphere. The resulting mixture was stirred at 110° C. for 3 h. After completion, the reaction mixture was cooled to room temperature and the solvent was removed under reduced pressure. The crude residue was purified by silica gel chromatography DCM/MeOH (19:1) to afford tert-butyl (5-(3,5-difluorophenyl)-6,7-dihydro-5H-pyrrolo[1,2-b][1,2,4]triazol-2-yl)carbamate (2.30 g, 93% yield) as a white solid.

LCMS (ESI) [M+H]⁺: 337.

### Step 8: (2R)-1-((tert-butoxycarbonyl)(5-(3,5-difluorophenyl)-6,7-dihydro-5H-pyrrolo[1,2-b][1,2,4]triazol-2-yl)amino)-1-oxopropan-2-yl acetate

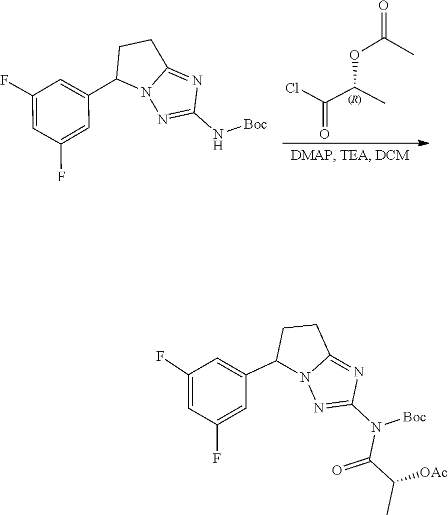

To a mixture of tert-butyl (5-(3,5-difluorophenyl)-6,7-dihydro-5H-pyrrolo[1,2-b][1,2,4]triazol-2-yl)carbamate (2.30 g, 6.8 mmol) in DCM (40 mL) were added DMAP (84 mg, 0.7 mmol), TEA (1.38 g, 13.7 mmol) and (2R)-1-chloro-1-oxopropan-2-yl acetate (1.54 g, 10.3 mmol) in portions at 0° C. under nitrogen atmosphere. The resulting mixture was stirred at 0° C. for 6 h. After completion, the solvent was removed under reduced pressure. The crude residue was purified by silica gel chromatograph PE/EA (1:1) to afford (2R)-1-((tert-butoxycarbonyl)(5-(3,5-difluorophenyl)-6,7-dihydro-5H-pyrrolo[1,2-b][1,2,4]triazol-2-yl)amino)-1-oxopropan-2-yl acetate (2.00 g, 65% yield) as a light yellow solid.

LCMS (ESI) [M+H]⁺: 451.

### Step 9: (2R)-1-((5-(3,5-difluorophenyl)-6,7-dihydro-5H-pyrrolo[1,2-b][1,2,4]triazol-2-yl)amino)-1-oxopropan-2-yl acetate

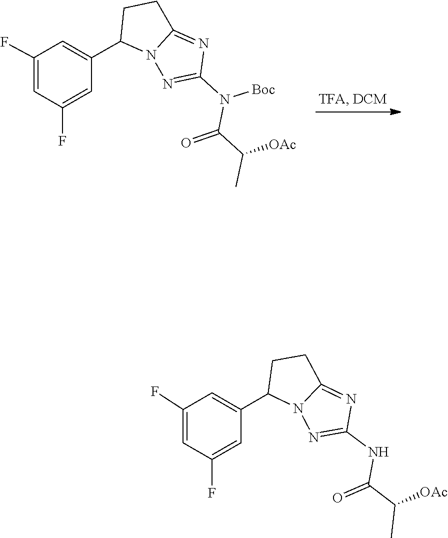

To a mixture of (2R)-1-((tert-butoxycarbonyl)(5-(3,5-difluorophenyl)-6,7-dihydro-5H-pyrrolo[1,2-b][1,2,4]triazol-2-yl)amino)-1-oxopropan-2-yl acetate (1.85 g, 5.5 mmol) in DCM (20 mL) was added trifluoroacetic acid (1.87 g, 16.4 mmol) dropwise at 0° C. The resulting mixture was stirred at room temperature for 2 h. After completion, the solvent was removed under reduced pressure. The crude residue was purified by reversed-phase flash chromatography (conditions: column, C18 silica gel; mobile phase, MeCN in Water (10 mmol/L NH₄HCO₃), 0% to 60% gradient in 25 min; detector, UV 240 nm) to give (2R)-1-((5-(3,5-difluorophenyl)-6,7-dihydro-5H-pyrrolo[1,2-b][1,2,4]triazol-2-yl)amino)-1-oxopropan-2-yl acetate (1.40 g, 57% yield) as a white solid.

LCMS (ESI) [M+H]⁺: 351.

### Step 10: (2R)—N-(5-(3,5-difluorophenyl)-6,7-dihydro-5H-pyrrolo[1,2-b][1,2,4]triazol-2-yl)-2-hydroxypropanamide

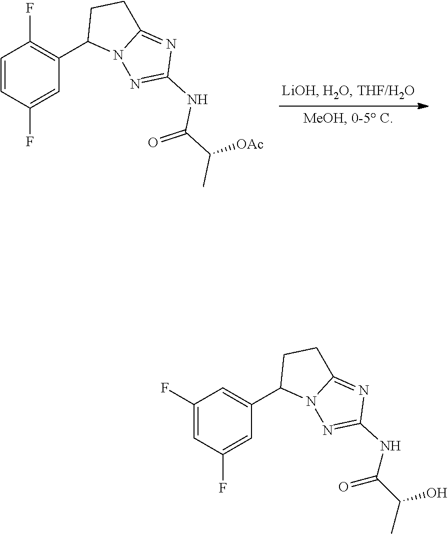 [SMILES: CC(=O)O[C@H](C)C(=O)Nc1nc2n(n1)C(c1cc(F)ccc1F)CC2 ; C[C@@H](O)C(=O)Nc1nc2n(n1)C(c1cc(F)cc(F)c1)CC2]

To a mixture of (2R)-1-((5-(3,5-difluorophenyl)-6,7-dihydro-5H-pyrrolo[1,2-b][1,2,4]triazol-2-yl)amino)-1-oxopropan-2-yl acetate (1.40 g, 4.0 mmol) in THF (6 mL) were added MeOH (6 mL), H₂O (6 mL) and LiOH monohydrate (293 mg, 7.0 mmol) at 0° C. under nitrogen atmosphere. The resulting mixture was stirred at room temperature for 2 h. After completion, the solvent was removed under reduced pressure. The crude residue was purified by reversed-phase flash chromatography (conditions: column, C18 silica gel; mobile phase, ACN in water (0.1% formic acid), 0% to 100% gradient in 25 min; detector, UV 240 nm) to give (2R)—N-(5-(3,5-difluorophenyl)-6,7-dihydro-5H-pyrrolo[1,2-b][1,2,4]triazol-2-yl)-2-hydroxypropanamide (1.13 g, 92% yield) as a white solid.

LCMS (ESI) [M+H]⁺: 309.

### Step 11: (2R)-1-((5-(3,5-difluorophenyl)-6,7-dihydro-5H-pyrrolo[1,2-b][1,2,4]triazol-2-yl)amino)-1-oxopropan-2-yl 2-nitrobenzenesulfonate

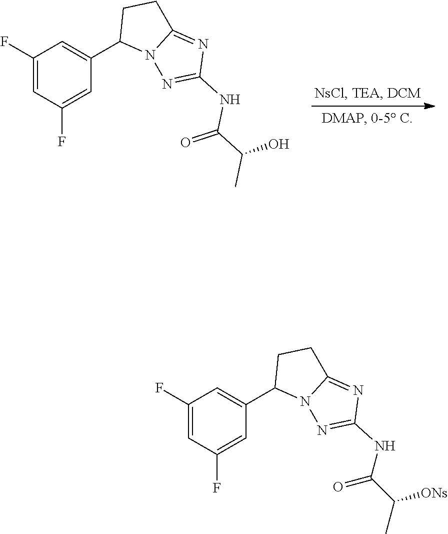

To a mixture of (2R)—N-(5-(3,5-difluorophenyl)-6,7-dihydro-5H-pyrrolo[1,2-b][1,2,4]triazol-2-yl)-2-hydroxypropanamide (400 mg, 1.3 mmol) in DCM (10 mL) were added DMAP (16 mg, 130 μmol), TEA (394 mg, 3.8 mmol) and 2-nitrobenzenesulfonyl chloride (288 mg, 1.3 mmol) at 0° C. under nitrogen atmosphere. The resulting mixture was stirred at 0° C. for 3 h. After completion, the solvent was removed under reduced pressure. The crude residue was purified by silica gel column chromatography, eluted with CH₂Cl₂/EA (1:1) to afford (2R)-1-((5-(3,5-difluorophenyl)-6,7-dihydro-5H-pyrrolo[1,2-b][1,2,4]triazol-2-yl)amino)-1-oxopropan-2-yl 2-nitrobenzenesulfonate (301 mg, 47% yield) as a white solid.

LCMS (ESI) [M+H]⁺: 494.

### Step 12: (2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-b][1,2,4]triazol-2-yl)propanamide

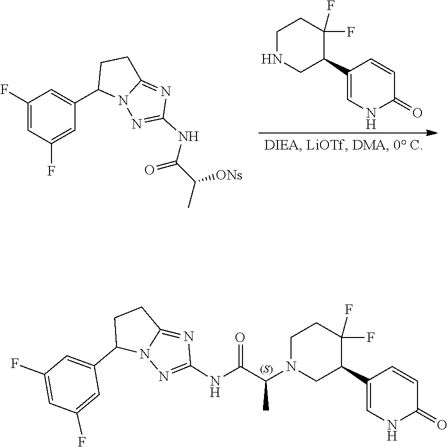

To a mixture of (2R)-1-((5-(3,5-difluorophenyl)-6,7-dihydro-5H-pyrrolo[1,2-b][1,2,4]triazol-2-yl)amino)-1-oxopropan-2-yl 2-nitrobenzenesulfonate (301 mg, 0.6 mmol) in N,N-dimethylacetamide (15 mL) were added DIEA (236 mg, 1.8 mmol), lithium trifluoromethanesulfonate (305 mg, 1.9 mmol) and (S)-5-(4,4-difluoropiperidin-3-yl)pyridin-2(1H)-one (131 mg, 0.6 mmol) at 0° C. under nitrogen atmosphere. The resulting mixture was stirred at 0° C. for 4 days. After completion, the reaction mixture was directly purified by reversed-phase flash chromatography (conditions: column, C18 silica gel; mobile phase, ACN in water (0.1% formic acid), 0% to 100% gradient in 30 min; detector, UV 254 nm) to give (2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-b][1,2,4]triazol-2-yl)propanamide (145 mg, 47% yield) as a white solid.

LCMS (ESI) [M+H]⁺: 505.

### Step 13: (S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-b][1,2,4]triazol-2-yl)propanamide and (S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-b][1,2,4]triazol-2-yl)propanamide

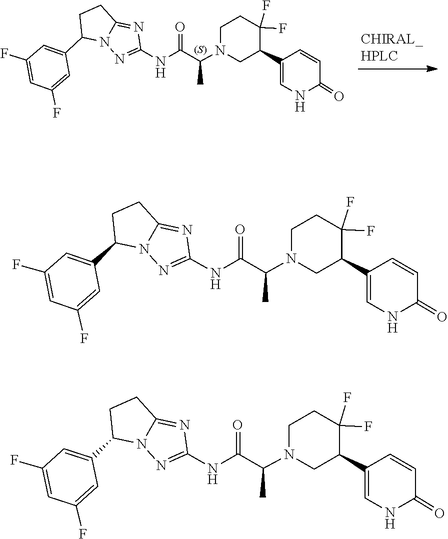

(2S)-2-((S)-4,4-difluoro-3-(6-oxo-1,6-dihydropyridin-3-yl)piperidin-1-yl)-N-(5-(3,5-difluorophenyl)-6,7-dihydro-5H-pyrrolo[1,2-b][1,2,4]triazol-2-yl)propanamide (145 mg) was purified by Prep-Chiral HPLC with the following conditions (Column: Lux Cellulose-2 4.6*50 cm 3 μm; Mobile Phase A: Hex (10 mM NH₃-MeOH), Mobile Phase B: EtOH:ACN=5:1; Flow rate: 20 mL/min; Gradient: isocratic 50% B; Wave Length: 220/254 nm; RT1 (min): 13.06; RT2 (min): 19.83) to afford the first peak (S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-b][1,2,4]triazol-2-yl)propanamide (Method Y, Peak 1, 2.01 min; 38.4 mg, 100% d.e. Compound 501) as a white solid and second peak (S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-b][1,2,4]triazol-2-yl)propanamide (Method Y, Peak 2, 3.72 min; 40.3 mg, 100% d.e. Compound 502) as a white solid.

TABLE 32

| Compound# | Characterization Data |
|---|---|
| 501 | LCMS (ESI) [M + H]⁺: 505.2 |
| | ¹H NMR (400 MHz, DMSO-d₆) δ 11.56 (bs, 1H), 10.22 (s, 1H), 7.39 (dd, J = 9.5, 2.6 Hz, 1H), |
| | 7.29-7.17 (m, 2H), 7.05-6.95 (m, 2H), 6.29 (d, J = 9.5 Hz, 1H), 5.50 (dd, J = 8.7, 5.6 Hz, |
| | 1H), 3.49 (bs, 1H), 3.24-2.98 (m, 3H), 2.98-2.70 (m, 4H), 2.49-2.41 (m, 2H), 2.14-1.90 |
| | (m, 2H), 1.16 (d, J = 6.9 Hz, 3H). |
| 502 | LCMS (ESI) [M + H]⁺: 505.2 |
| | ¹H NMR (400 MHz, DMSO-d₆) δ 11.56 (bs, 1H), 10.27 (s, 1H), 7.39 (dd, J = 9.4, 2.6 Hz, 1H), |
| | 7.33-7.17 (m, 2H), 7.06-6.98 (m, 2H), 6.29 (d, J = 9.4 Hz, 1H), 5.50 (dd, J = 8.7, 5.7 Hz, |
| | 1H), 3.51 (bs, 1H), 3.24-2.99 (m, 3H), 2.99-2.70 (m, 4H), 2.46 (m, 2H), 2.14-1.91 (m, |
| | 2H), 1.16 (d, J = 6.8 Hz, 3H). |

### Example 59: (2S)-2-((S)-4,4-difluoro-3-(6-oxo-1,6-dihydropyridin-3-yl)piperidin-1-yl)-N-(5-(2,5-difluorophenyl)-6,7-dihydro-5H-pyrrolo[1,2-a]imidazol-2-yl)propanamide

### Step 1: 5-(2,5-difluorophenyl)pyrrolidin-2-one

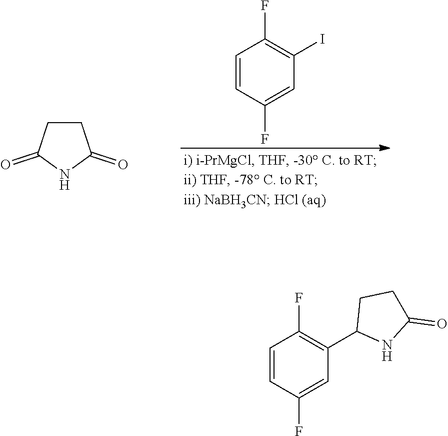 [SMILES: O=C1CCC(=O)N1 ; Fc1ccc(F)c(I)c1 ; O=C1CCC(c2cc(F)ccc2F)N1]

To a mixture of 1,4-difluoro-2-iodobenzene (26.60 g, 111.0 mmol) in THF (250 mL) was added isopropylmagnesium chloride-lithium chloride complex (1.3 M in THF, 85.5 mL) dropwise over 12 min at −30° C. under nitrogen atmosphere. The resulting mixture was stirred at room temperature for 1 h under nitrogen atmosphere. To the above mixture was added succinimide (5.00 g, 50.5 mmol) dropwise over 15 min at −78° C. The resulting mixture was stirred at room temperature for additional 2 h. To the above mixture was added NaBH₃CN (3.8 g, 60.6 mmol) in portions over 12 min at 0° C. The resulting mixture was stirred at room temperature for additional 30 min. The mixture was acidified to pH 3 with aqueous HCl (1 M) at 0° C. The resulting mixture was stirred at room temperature for 1.5 h under nitrogen atmosphere. The mixture was neutralized to pH 7 with ammonium hydroxide. The resulting mixture was extracted with EtOAc (3×300 mL). The combined organic layers were dried over anhydrous Na₂SO₄, filtered and concentrated under reduced pressure. The crude residue was purified by silica gel column chromatography, eluted with CH₂Cl₂/MeOH (10:1) to afford 5-(2,5-difluorophenyl)pyrrolidin-2-one (3.30 g, 33% yield) as a yellow solid.

LCMS (ESI) [M+H]⁺: 198.

### Step 2: 2-(2-(2,5-difluorophenyl)-5-oxopyrrolidin-1-yl)acetamide

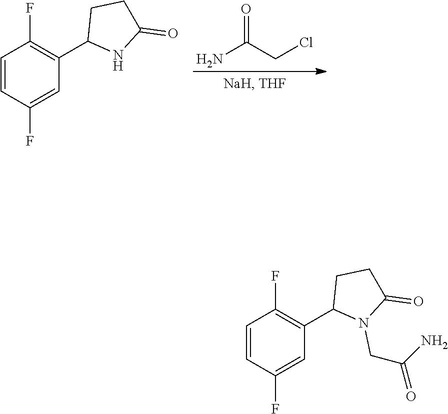 [SMILES: O=C1CCC(c2cc(F)ccc2F)N1 ; NC(=O)CCl ; NC(=O)CN1C(=O)CCC1c1cc(F)ccc1F]

To a mixture of 5-(2,5-difluorophenyl)pyrrolidin-2-one (3.30 g, 16.7 mmol) in THF (35 mL) was added NaH (60% in mineral oil, 1.20 g, 50.2 mmol) in portions at 0° C. under nitrogen atmosphere. The resulting mixture was stirred at room temperature for 30 min under nitrogen atmosphere. To the above mixture was added 2-chloroacetamide (1.72 g, 18.4 mmol) dropwise over 5 min at 0° C. The resulting mixture was stirred at room temperature for additional 3 h. The reaction was quenched with water at 0° C. The resulting mixture was extracted with EtOAc (3×40 mL). The combined organic layers were dried over anhydrous Na₂SO₄, filtered, and concentrated under reduced pressure. The crude residue was purified by silica gel column chromatography, eluted with CH₂Cl₂/MeOH (15:1) to afford 2-(2-(2,5-difluorophenyl)-5-oxopyrrolidin-1-yl)acetamide (2.10 g, 67% yield) as a yellow oil.

LCMS (ESI) [M+H]⁺: 255.

### Step 3: 2-bromo-5-(2,5-difluorophenyl)-6,7-dihydro-5H-pyrrolo[1,2-a]imidazole

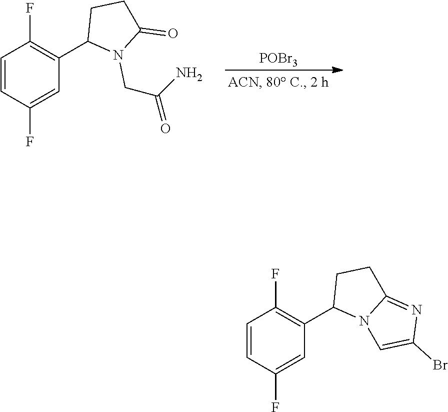 [SMILES: NC(=O)CN1C(=O)CCC1c1cc(F)ccc1F ; "POBr3" ; Fc1ccc(F)c(C2CCc3nc(Br)cn32)c1]

To a mixture of 2-(2-(2,5-difluorophenyl)-5-oxopyrrolidin-1-yl)acetamide (2.00 g, 7.9 mmol) in ACN (40 mL) was added phosphoroyl bromide (9.00 g, 31.5 mmol) at 0° C. under nitrogen atmosphere. The resulting mixture was stirred at 80° C. for 2 h under nitrogen atmosphere. The mixture was allowed to cool down to room temperature. The mixture was neutralized to pH 7 with ammonium hydroxide solution. The resulting mixture was extracted with EtOAc (3×50 mL). The combined organic layers were dried over anhydrous Na₂SO₄, filtered, and concentrated under reduced pressure. The crude residue was purified by silica gel column chromatography, eluted with CH₂Cl₂/MeOH (13:1) to afford 2-bromo-5-(2,5-difluorophenyl)-6,7-dihydro-5H-pyrrolo[1,2-a]imidazole (1.80 g, 76% yield) as a yellow solid.

LCMS (ESI) [M+H]⁺: 299.

### Step 4: (2R)—N-(5-(2,5-difluorophenyl)-6,7-dihydro-5H-pyrrolo[1,2-a]imidazol-2-yl)-2-hydroxypropanamide

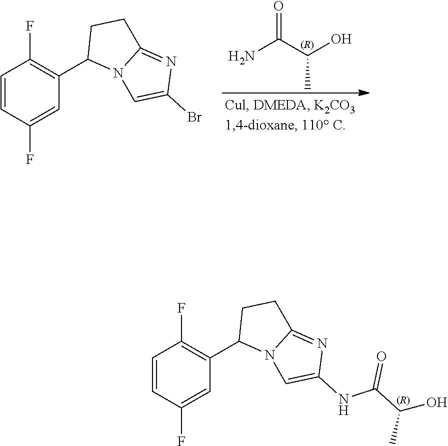

To a mixture of (R)-2-hydroxypropanamide (1.00 g, 12.0 mmol) in 1,4-dioxane (15 mL) were added CuI (229 mg, 1.2 mmol), K₂CO₃ (1.66 g, 12.0 mmol), 1,2-bis(dimethylamino)ethane (212 mg, 2.4 mmol) and 2-bromo-5-(2,5-difluorophenyl)-6,7-dihydro-5H-pyrrolo[1,2-a]imidazole (1.80 g, 6.0 mmol). The resulting mixture was stirred at 110° C. overnight under nitrogen atmosphere. The mixture was allowed to cool down to room temperature. The reaction was quenched with water at 0° C. The resulting mixture was extracted with EtOAc (3×20 mL). The combined organic layers were dried over anhydrous Na₂SO₄, filtered, and concentrated under reduced pressure. The crude residue was purified by silica gel column chromatography, eluted with DCM/MeOH (18:1) to afford (2R)—N-(5-(2,5-difluorophenyl)-6,7-dihydro-5H-pyrrolo[1,2-a]imidazol-2-yl)-2-hydroxypropanamide (1.20 g, 64% yield) as a yellow oil.

LCMS (ESI) [M+H]⁺: 308.

### Step 5: (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate

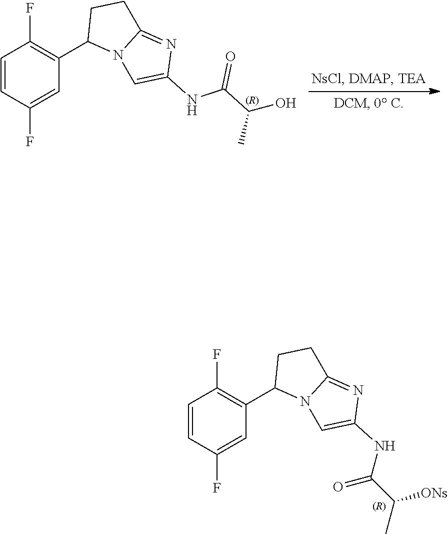

To a mixture of (2R)—N-(5-(2,5-difluorophenyl)-6,7-dihydro-5H-pyrrolo[1,2-a]imidazol-2-yl)-2-hydroxypropanamide (400 mg, 1.3 mmol) in DCM (6 mL) were added DMAP (31 mg, 0.3 mmol), TEA (395 mg, 3.9 mmol) and 2-nitrobenzene-1-sulfonyl chloride (317 mg, 1.4 mmol) in portions at 0° C. under nitrogen atmosphere. The resulting mixture was stirred at 0° C. for 4 h under nitrogen atmosphere. The reaction was quenched with water at 0° C. The resulting mixture was extracted with EtOAc (3×8 mL). The combined organic layers were dried over anhydrous Na₂SO₄, filtered, and concentrated under reduced pressure. The crude residue was purified by silica gel column chromatography, eluted with DCM/EA (1:1) to afford (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate (160 mg, 25% yield) as a yellow solid.

LCMS (ESI) [M+H]⁺: 493.

### Step 6: (2S)-2-((S)-4,4-difluoro-3-(6-oxo-1,6-dihydropyridin-3-yl)piperidin-1-yl)-N-(5-(2,5-difluorophenyl)-6,7-dihydro-5H-pyrrolo[1,2-a]imidazol-2-yl)propanamide

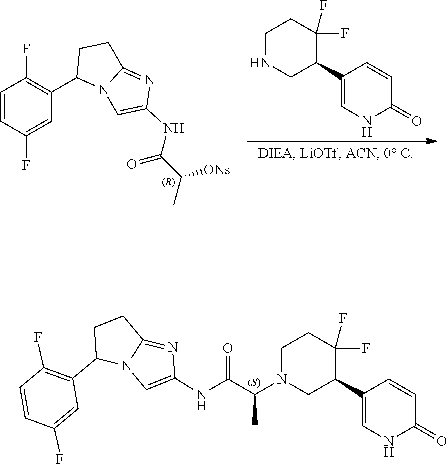

To a mixture of (S)-5-(4,4-difluoropiperidin-3-yl)pyridin-2(1H)-one (48 mg, 0.2 mmol) in ACN (5 mL) were added DIEA (78 mg, 0.6 mmol), lithium trifluoromethanesulfonate (95 mg, 0.6 mmol) and (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate (100 mg, 0.2 mmol) dropwise at 0° C. under nitrogen atmosphere. The resulting mixture was stirred at 0° C. overnight under nitrogen atmosphere. The resulting mixture was concentrated under reduced pressure. The crude residue was purified by reversed-phase flash chromatography (conditions: column, C18 silica gel; mobile phase, ACN in water (0.1% formic acid), 0% to 100% gradient in 30 min; detector, UV 254 nm) to give (2S)-2-((S)-4,4-difluoro-3-(6-oxo-1,6-dihydropyridin-3-yl)piperidin-1-yl)-N-(5-(2,5-difluorophenyl)-6,7-dihydro-5H-pyrrolo[1,2-a]imidazol-2-yl)propanamide (40 mg, 39% yield) as a white solid.

LCMS (ESI) [M+H]⁺: 504.

### Step 7: (S)-2-((S)-4,4-difluoro-3-(6-oxo-1,6-dihydropyridin-3-yl)piperidin-1-yl)-N—((R)-5-(2,5-difluorophenyl)-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N—((S)-5-(2,5-difluorophenyl)-6,7-dihydro-5H-pyrrolo[1,2-a]imidazol-2-yl)propanamide

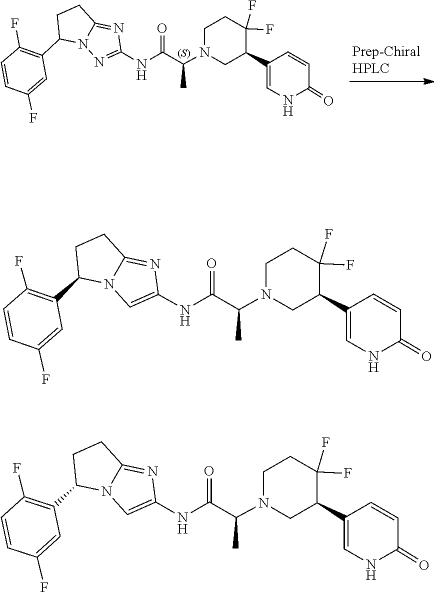

(2S)-2-((S)-4,4-difluoro-3-(6-oxo-1,6-dihydropyridin-3-yl)piperidin-1-yl)-N-(5-(2,5-difluorophenyl)-6,7-dihydro-5H-pyrrolo[1,2-a]imidazol-2-yl)propanamide (40 mg) was separated by Prep-Chiral HPLC with the following conditions (Column: Lux 5 μm Cellulose-4 3*25 cm, 5 μm; Mobile Phase A: Hex (10 mM NH₃-MeOH), Mobile Phase B: EtOH:ACN=5:1; Flow rate: 40 mL/min; Gradient: isocratic 40% B; Wave Length: 220/254 nm; RT1 (min): 10.06; RT2 (min): 12.88) to afford first peak (S)-2-((S)-4,4-difluoro-3-(6-oxo-1,6-dihydropyridin-3-yl)piperidin-1-yl)-N—((R)-5-(2,5-difluorophenyl)-6,7-dihydro-5H-pyrrolo[1,2-a]imidazol-2-yl)propanamide (Method Y, Peak 2, 1.62 min; 8.0 mg, 100% d.e.) (Compound 503) as a white solid and second peak (S)-2-((S)-4,4-difluoro-3-(6-oxo-1,6-dihydropyridin-3-yl)piperidin-1-yl)-N—((S)-5-(2,5-difluorophenyl)-6,7-dihydro-5H-pyrrolo[1,2-a]imidazol-2-yl)propanamide (Method Y, Peak 1, 1.44 min; 8.0 mg, 100% d.e.) (Compound 504) as a white solid.

TABLE 33

| Compound# | Characterization Data |
|---|---|
| 503 | LCMS (ESI) [M + H]⁺: 504.2 |
| | ¹H NMR (400 MHz, DMSO-d₆) δ 11.56 (bs, 1H), 10.15 (s, 1H), 7.42-7.33 (m, 2H), 7.25 (d, |
| | J = 12.9, 4.3 Hz, 2H), 7.02 (s, 1H), 6.87-6.76 (m, 1H), 6.29 (d, J = 9.5 Hz, 1H), 5.57 (dd, |
| | J = 8.3, 4.8 Hz, 1H), 3.52 (d, J = 6.8 Hz, 1H), 3.21-3.04 (m, 1H), 3.04-2.94 (m, 1H), 2.93- |
| | 2.78 (m, 5H), 2.51-2.40 (m, 2H), 2.12-1.89 (m, 2H), 1.15 (d, J = 6.9 Hz, 3H). |
| 504 | LCMS (ESI) [M + H]⁺: 504.3 |
| | ¹H NMR (400 MHz, DMSO-d₆) δ 11.57 (bs, 1H), 10.08 (s, 1H), 7.42-7.22 (m, 4H), 7.00 (s, |
| | 1H), 6.91-6.84 (m, 1H), 6.29 (d, J = 9.5 Hz, 1H), 5.57 (dd, J = 8.3, 4.8 Hz, 1H), 3.61-3.47 |
| | (m, 1H), 3.25-3.08 (m, 1H), 3.06-2.75 (m, 6H), 2.50-2.38 (m, 2H), 2.14-1.91 (m, 2H), |
| | 1.16 (d, J = 6.9 Hz, 3H). |

### Example 60: (2S)-2-((S)-4,4-difluoro-3-(6-oxo-1,6-dihydropyridin-3-yl)piperidin-1-yl)-N-(5-(2,4-difluorophenyl)-6,7-dihydro-5H-pyrrolo[1,2-a]imidazol-2-yl)propanamide

### Step 1: 5-(2,4-difluorophenyl)pyrrolidin-2-one

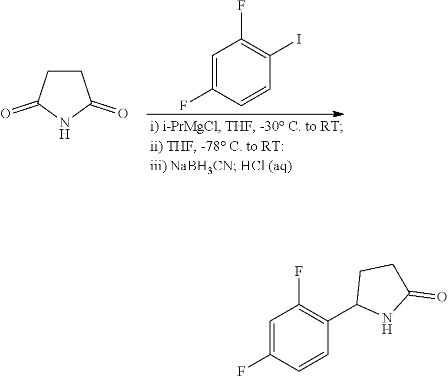 [SMILES: O=C1CCC(=O)N1 ; Fc1ccc(I)c(F)c1 ; O=C1CCC(c2ccc(F)cc2F)N1]

To a mixture of 2,4-difluoro-1-iodobenzene (12.10 g, 50.0 mmol) in THF (120 mL) was added isopropylmagnesium chloride-lithium chloride complex (1.3 M in THF, 7.30 g, 50.0 mmol) dropwise over 10 min at −30° C. under nitrogen atmosphere. The resulting mixture was stirred at room temperature for 1 h under nitrogen atmosphere. To the above mixture was added succinimide (5.00 g, 50.2 mmol) dropwise over 15 min at −78° C. The resulting mixture was stirred at room temperature for additional 2 h. To the above mixture was added NaBH₃CN (6.30 g, 100.9 mmol) in portions over 10 min at 0° C. The resulting mixture was stirred at room temperature for additional 30 min. The mixture was acidified to pH 3 with aqueous HCl (1 M) at 0° C. The resulting mixture was stirred at room temperature for 1.5 h under nitrogen atmosphere. The mixture was neutralized to pH 7 with ammonium hydroxide. The resulting mixture was extracted with EtOAc (3×200 mL). The combined organic layers were dried over anhydrous Na₂SO₄, filtered, and concentrated under reduced pressure. The crude residue was purified by silica gel column chromatography, eluted with PE/EA (1:1) to afford 5-(2,4-difluorophenyl)pyrrolidin-2-one (3.00 g, 30% yield) as an off-white solid.

LCMS (ESI) [M+H]⁺: 198.

### Step 2: 2-(2-(2,4-difluorophenyl)-5-oxopyrrolidin-1-yl)acetamide

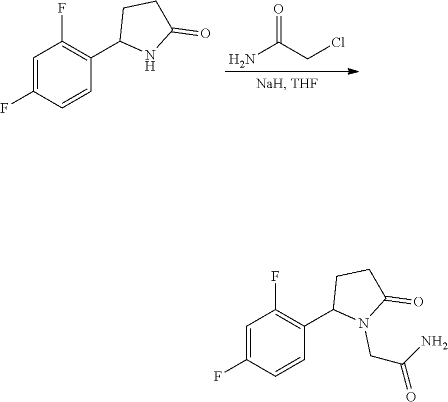 [SMILES: O=C1CCC(c2ccc(F)cc2F)N1 ; NC(=O)CCl ; NC(=O)CN1C(=O)CCC1c1ccc(F)cc1F]

To a mixture of 5-(2,4-difluorophenyl)pyrrolidin-2-one (3.00 g, 15.2 mmol) in THF (25 mL) was added NaH (60% in mineral oil, 1.20 g, 30.4 mmol) in portions at 0° C. under nitrogen atmosphere. The resulting mixture was stirred at room temperature for 30 min under nitrogen atmosphere. To the above mixture was added chloroacetamide (1.70 g, 18.2 mmol) dropwise over 5 min at 0° C. The resulting mixture was stirred at room temperature for additional 3 h. The reaction was quenched with water at 0° C. The resulting mixture was extracted with EtOAc (3×30 mL). The combined organic layers were dried over anhydrous Na₂SO₄, filtered and concentrated under reduced pressure. The crude residue was purified by silica gel column chromatography, eluted with PE/EA (1:1) to afford 2-(2-(2,4-difluorophenyl)-5-oxopyrrolidin-1-yl)acetamide (1.90 g, 45% yield) as a light yellow solid.

LCMS (ESI) [M+H]⁺: 255.

### Step 3: 2-bromo-5-(2,4-difluorophenyl)-6,7-dihydro-5H-pyrrolo[1,2-a]imidazole

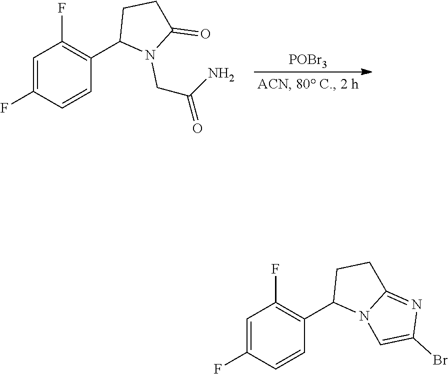 [SMILES: NC(=O)CN1C(=O)CCC1c1ccc(F)cc1F ; "POBr3" ; Fc1ccc(C2CCc3nc(Br)cn32)c(F)c1]

To a mixture of 2-(2-(2,4-difluorophenyl)-5-oxopyrrolidin-1-yl)acetamide (1.90 g, 7.4 mmol) in ACN (40 mL) was added phosphoroyl bromide (8.4 g, 29.6 mmol) at 0° C. under nitrogen atmosphere. The resulting mixture was stirred at 80° C. for 2 h under nitrogen atmosphere. The mixture was allowed to cool down to room temperature. The mixture was neutralized to pH 7 with ammonium hydroxide solution. The resulting mixture was extracted with EtOAc (3×50 mL). The combined organic layers were dried over anhydrous Na₂SO₄, filtered and concentrated under reduced pressure. The crude residue was purified by silica gel column chromatography, eluted with CH₂Cl₂/MeOH (10:1) to afford 2-bromo-5-(2,4-difluorophenyl)-6,7-dihydro-5H-pyrrolo[1,2-a]imidazole (2.10 g, 93% yield) as a yellow solid.

LCMS (ESI) [M+H]⁺: 299.

### Step 4: (2R)—N-(5-(2,4-difluorophenyl)-6,7-dihydro-5H-pyrrolo[1,2-a]imidazol-2-yl)-2-hydroxypropanamide

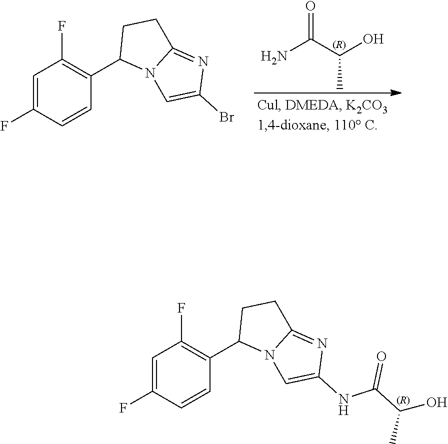

To a mixture of (R)-2-hydroxypropanamide (149 mg, 1.7 mmol) in 1,4-dioxane (8 mL) were added CuI (64 mg, 0.3 mmol), 2-bromo-5-(2,4-difluorophenyl)-6,7-dihydro-5H-pyrrolo[1,2-a]imidazole (500 mg, 1.7 mmol), K₂CO₃ (462 mg, 3.3 mmol) and 1,2-bis(dimethylamino)ethane (59 mg, 0.7 mmol). The resulting mixture was stirred at 110° C. for 16 h under nitrogen atmosphere. The mixture was allowed to cool down to room temperature. The reaction was quenched with water at 0° C. The resulting mixture was extracted with EtOAc (3×10 mL). The combined organic layers were dried over anhydrous Na₂SO₄, filtered and concentrated under reduced pressure. The crude residue was purified by silica gel column chromatography, eluted with DCM/MeOH (12:1) to afford (2R)—N-(5-(2,4-difluorophenyl)-6,7-dihydro-5H-pyrrolo[1,2-a]imidazol-2-yl)-2-hydroxypropanamide (300 mg, 58% yield) as a yellow solid.

LCMS (ESI) [M+H]⁺: 308.

### Step 5: (2R)-1-((5-(2,4-difluorophenyl)-6,7-dihydro-5H-pyrrolo[1,2-a]imidazol-2-yl)amino)-1-oxopropan-2-yl 2-nitrobenzenesulfonate

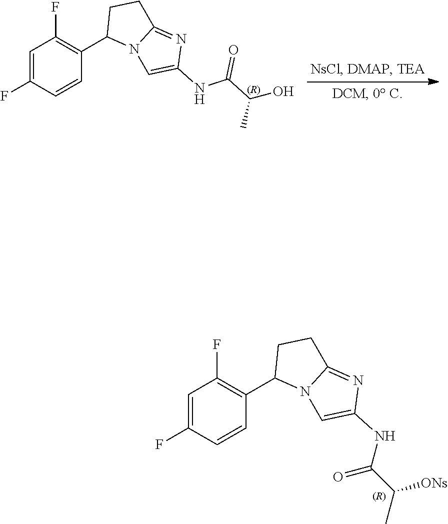

To a mixture of (2R)—N-(5-(2,4-difluorophenyl)-6,7-dihydro-5H-pyrrolo[1,2-a]imidazol-2-yl)-2-hydroxypropanamide (300 mg, 0.9 mmol) in DCM (6 mL) were added DMAP (24 mg, 0.2 mmol), TEA (198 mg, 2.0 mmol) and 2-nitrobenzene-1-sulfonyl chloride (238 mg, 1.1 mmol) in portions at 0° C. under nitrogen atmosphere. The resulting mixture was stirred at 0° C. for 4 h under nitrogen atmosphere. The reaction was quenched with water at 0° C. The resulting mixture was extracted with EtOAc (3×8 mL). The combined organic layers were dried over anhydrous Na₂SO₄, filtered and concentrated under reduced pressure. The crude residue was purified by silica gel column chromatography, eluted with DCM/EA (1:1) to afford (2R)-1-((5-(2,4-difluorophenyl)-6,7-dihydro-5H-pyrrolo[1,2-a]imidazol-2-yl)amino)-1-oxopropan-2-yl 2-nitrobenzenesulfonate (180 mg, 37% yield) as a yellow solid.

LCMS (ESI) [M+H]⁺: 493.

### Step 6: (2S)-2-((S)-4,4-difluoro-3-(6-oxo-1,6-dihydropyridin-3-yl)piperidin-1-yl)-N-(5-(2,4-difluorophenyl)-6,7-dihydro-5H-pyrrolo[1,2-a]imidazol-2-yl)propanamide

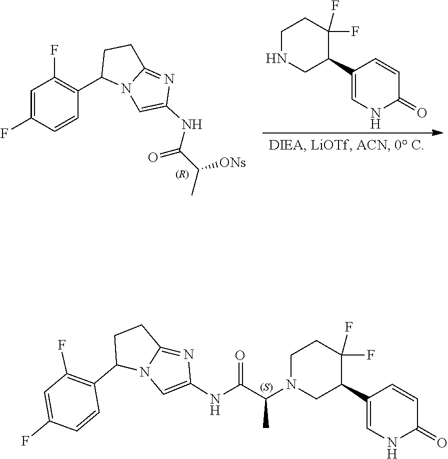

To a mixture of (S)-5-(4,4-difluoropiperidin-3-yl)pyridin-2(1H)-one (35 mg, 162 μmol) in ACN (5 mL) were added DIEA (73 mg, 567 μmol), lithium trifluoromethanesulfonate (88 mg, 567 μmol) and (2R)-1-((5-(2,4-difluorophenyl)-6,7-dihydro-5H-pyrrolo[1,2-a]imidazol-2-yl)amino)-1-oxopropan-2-yl 2-nitrobenzenesulfonate (80 mg, 162 μmol) dropwise at 0° C. under nitrogen atmosphere. The resulting mixture was stirred at 0° C. overnight under nitrogen atmosphere. The resulting mixture was concentrated under reduced pressure. The crude residue was purified by reversed-phase flash chromatography (conditions: column, C18 silica gel; mobile phase, ACN in water (0.1% formic acid), 0% to 100% gradient in 30 min; detector, UV 254 nm) to give (2S)-2-((S)-4,4-difluoro-3-(6-oxo-1,6-dihydropyridin-3-yl)piperidin-1-yl)-N-(5-(2,4-difluorophenyl)-6,7-dihydro-5H-pyrrolo[1,2-a]imidazol-2-yl)propanamide (50 mg, 61% yield) as an off-white solid.

LCMS (ESI) [M+H]⁺: 504.

### Step 7: (S)-2-((S)-4,4-difluoro-3-(6-oxo-1,6-dihydropyridin-3-yl)piperidin-1-yl)-N—((R)-5-(2,4-difluorophenyl)-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N—((S)-5-(2,4-difluorophenyl)-6,7-dihydro-5H-pyrrolo[1,2-a]imidazol-2-yl)propanamide

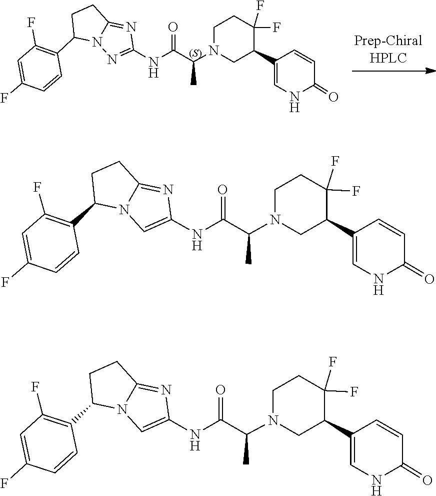

(2S)-2-((S)-4,4-difluoro-3-(6-oxo-1,6-dihydropyridin-3-yl)piperidin-1-yl)-N-(5-(2,4-difluorophenyl)-6,7-dihydro-5H-pyrrolo[1,2-a]imidazol-2-yl)propanamide (50 mg) was separated by Prep-Chiral HPLC with the following conditions (Column: Lux 5 μm Cellulose-4 3*25 cm, 5 μm; Mobile Phase A: Hex (10 mM NH₃-MeOH), Mobile Phase B: EtOH:ACN=5:1; Flow rate: 40 mL/min; Gradient: isocratic 30% B; Wave Length: 232/212 nm; RT1 (min): 14.83; RT2 (min): 18.49) to afford first peak (S)-2-((S)-4,4-difluoro-3-(6-oxo-1,6-dihydropyridin-3-yl)piperidin-1-yl)-N—((R)-5-(2,4-difluorophenyl)-6,7-dihydro-5H-pyrrolo[1,2-a]imidazol-2-yl)propanamide (Method Y, Peak 2, 1.59 min; 15.1 mg, 100% d.e.) (Compound 505) as a white solid and second peak (S)-2-((S)-4,4-difluoro-3-(6-oxo-1,6-dihydropyridin-3-yl)piperidin-1-yl)-N—((S)-5-(2,4-difluorophenyl)-6,7-dihydro-5H-pyrrolo[1,2-a]imidazol-2-yl)propanamide (Method Y, Peak 1, 1.45 min; 15.8 mg, 100% d.e.) (Compound 506) as a white solid.

TABLE 34

| Compound# | Characterization Data |
|---|---|
| 505 | LCMS (ESI) [M + H]⁺: 504.2 |
| | ¹H NMR (400 MHz, DMSO-d₆) δ 11.58 (bs, 1H), 10.22 (s, 1H), 7.46-7.26 (m, 3H), 7.17- |
| | 7.06 (m, 2H), 7.00 (d, J = 1.4 Hz, 1H), 6.29 (d, J = 9.4 Hz, 1H), 5.58 (dd, J = 8.2, 5.0 Hz, |
| | 1H), 3.58-3.46 (m, 1H), 3.20-3.05 (m, 1H), 3.04-2.72 (m, 6H), 2.52-2.33 (m, 2H), 2.14- |
| | 1.89 (m, 2H), 1.18 (s, 3H). |
| 506 | LCMS (ESI) [M + H]⁺: 504.2 |
| | ¹H NMR (400 MHz, DMSO-d₆) δ 11.60 (bs, 1H), 10.25 (s, 1H), 7.47-7.27 (m, 3H), 7.17- |
| | 7.06 (m, 2H), 7.00 (s, 1H), 6.29 (d, J = 9.5 Hz, 1H), 5.61-5.53 (m, 1H), 3.56-3.48 (m, 1H), |
| | 3.21-3.09 (m, 1H), 3.05-2.76 (m, 6H), 2.52-2.31 (m, 2H), 2.16-1.98 (m, 2H), 1.20 (d, |
| | J = 8.5 Hz, 3H). |

### Example 61: (2S)-2-((S)-4,4-difluoro-3-(6-oxo-1,6-dihydropyridin-3-yl)piperidin-1-yl)-N-(5-(3,5-difluorophenyl)-5,6,7,8-tetrahydroimidazo[1,2-a]pyridin-2-yl)propanamide

### Step 1: 5-(3,5-difluorophenyl)imidazo[1,2-a]pyridine

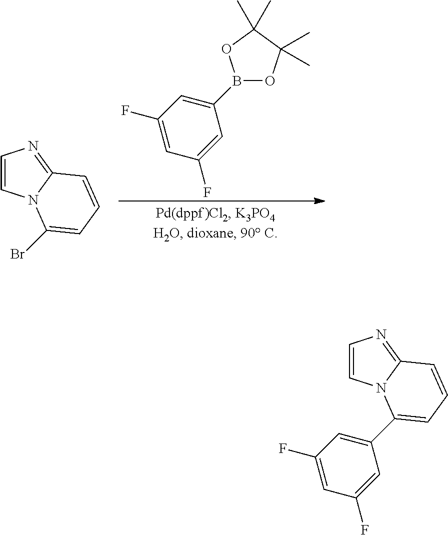 [SMILES: Brc1cccc2nccn12 ; CC1(C)OB(c2cc(F)cc(F)c2)OC1(C)C ; Fc1cc(F)cc(-c2cccc3nccn23)c1]

A solution of 5-bromoimidazo[1,2-a]pyridine (2.00 g, 10.2 mmol), 2-(3,5-difluorophenyl)-4,4,5,5-tetramethyl-1,3,2-dioxaborolane (3.66 g, 15.2 mmol), Pd(dppf)Cl₂ (743 mg, 1.0 mmol) and K₃PO₄ (6.46 g, 30.5 mmol) in dioxane (30 mL)/H₂O (6 mL) was stirred at 90° C. for 3 h under nitrogen atmosphere. The mixture was allowed to cool down to room temperature. The reaction was diluted with water at 0° C. The aqueous layer was extracted with EtOAc (3×50 mL). The combined organic layers were dried over anhydrous Na₂SO₄, filtered and concentrated under reduced pressure. The crude residue was purified by silica gel column chromatography, eluted with PE/EA (1:1) to afford 5-(3,5-difluorophenyl)imidazo[1,2-a]pyridine (2.00 g, 86% yield) as a light yellow solid.

LCMS (ESI) [M+H]⁺: 231.

### Step 2: 5-(3,5-difluorophenyl)-5,6,7,8-tetrahydroimidazo[1,2-a]pyridine

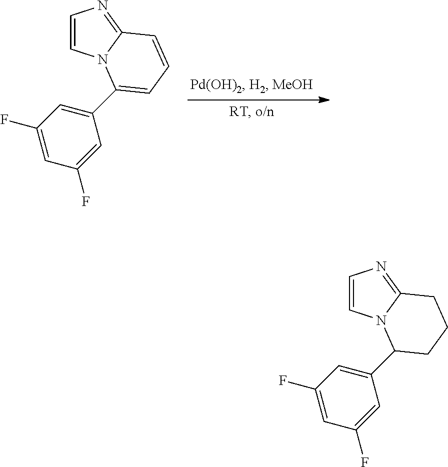 [SMILES: Fc1cc(F)cc(-c2cccc3nccn23)c1 ; Fc1cc(F)cc(C2CCCc3nccn32)c1]

A solution of 5-(3,5-difluorophenyl)imidazo[1,2-a]pyridine (2.00 g, 8.7 mmol) and palladium hydroxide (244 mg, 1.7 mmol) in MeOH (30 mL) was stirred at room temperature overnight under hydrogen atmosphere. The resulting mixture was filtered through a short plug of celite using methanol. The filter cake was washed with MeOH (3×50 mL). The filtrate was concentrated under reduced pressure. The crude residue was purified by reversed-phase flash chromatography (conditions: column, C18 silica gel; mobile phase, ACN in water (0.1% formic acid), 0% to 100% gradient in 25 min; detector, UV 254 nm) to give 5-(3,5-difluorophenyl)-5,6,7,8-tetrahydroimidazo[1,2-a]pyridine (1.20 g, 59% yield) as a light yellow oil.

LCMS (ESI) [M+H]⁺: 235.

### Step 3: 2,3-dibromo-5-(3,5-difluorophenyl)-5,6,7,8-tetrahydroimidazo[1,2-a]pyridine

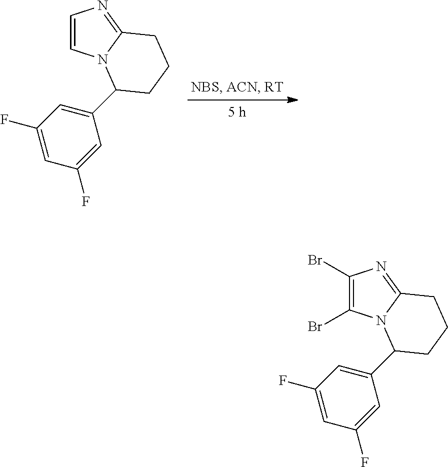 [SMILES: Fc1cc(F)cc(C2CCCc3nccn32)c1 ; Fc1cc(F)cc(C2CCCc3nc(Br)c(Br)n32)c1]

A solution of 5-(3,5-difluorophenyl)-5,6,7,8-tetrahydroimidazo[1,2-a]pyridine (1.20 g, 5.1 mmol) and NBS (2.28 g, 12.8 mmol) in ACN (20 mL) was stirred at room temperature for 5 h under nitrogen atmosphere. The reaction was quenched with water at 0° C. The aqueous layer was extracted with CH₂Cl₂ (3×30 mL). The combined organic layers were dried over anhydrous Na₂SO₄, filtered and concentrated under reduced pressure. The crude residue was purified by silica gel column chromatography, eluted with PE/EA (1:2) to afford 2,3-dibromo-5-(3,5-difluorophenyl)-5,6,7,8-tetrahydroimidazo[1,2-a]pyridine (600 mg, 30% yield) as a light yellow solid.

LCMS (ESI) [M+H]⁺: 391.

### Step 4: 2-bromo-5-(3,5-difluorophenyl)-5,6,7,8-tetrahydroimidazo[1,2-a]pyridine

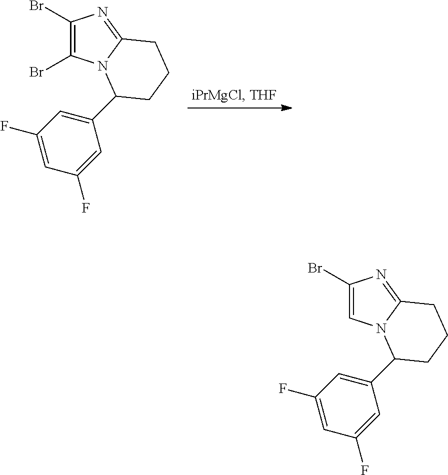 [SMILES: Fc1cc(F)cc(C2CCCc3nc(Br)c(Br)n32)c1 ; Fc1cc(F)cc(C2CCCc3nc(Br)cn32)c1]

To a mixture of 2,3-dibromo-5-(3,5-difluorophenyl)-5,6,7,8-tetrahydroimidazo[1,2-a]pyridine (600 mg, 1.5 mmol) in THF (10 mL) was added isopropylmagnesium chloride-lithium chloride complex (1.4 mL, 1.8 mmol, 1.3 M in THF) dropwise over 5 min at 0° C. The resulting mixture was stirred at room temperature for 3 h under nitrogen atmosphere. The reaction mixture was quenched with NH₄Cl solution (12 mL) at 0° C. The aqueous layer was extracted with CH₂Cl₂ (3×15 mL). The combined organic layers were dried over anhydrous Na₂SO₄, filtered and concentrated under reduced pressure. The crude residue was purified by silica gel column chromatography, eluted with PE/EA (1:2) to afford 2-bromo-5-(3,5-difluorophenyl)-5,6,7,8-tetrahydroimidazo[1,2-a]pyridine (400 mg, 83% yield) as a light yellow solid.

LCMS (ESI) [M+H]⁺: 313.

### Step 5: (2R)—N-(5-(3,5-difluorophenyl)-5,6,7,8-tetrahydroimidazo[1,2-a]pyridin-2-yl)-2-hydroxypropanamide

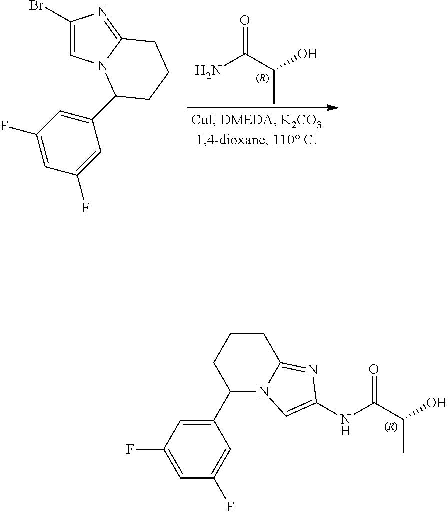

A solution of 2-bromo-5-(3,5-difluorophenyl)-5,6,7,8-tetrahydroimidazo[1,2-a]pyridine (400 mg, 1.3 mmol), (2R)-2-hydroxypropanamide (171 mg, 1.9 mmol), CuI (49 mg, 0.3 mmol), K₂CO₃ (353 mg, 2.6 mmol) and 1,2-bis(dimethylamino)ethane (45 mg, 0.5 mmol) in 1,4-dioxane (8 mL) was stirred at 110° C. overnight under nitrogen atmosphere. The mixture was allowed to cool down to room temperature. The reaction was quenched with water at 0° C. The aqueous layer was extracted with EtOAc (3×10 mL). The combined organic layers were dried over anhydrous Na₂SO₄, filtered and concentrated under reduced pressure. The crude residue was purified by silica gel column chromatography, eluted with PE/EA (1:1) to afford (2R)—N-(5-(3,5-difluorophenyl)-5,6,7,8-tetrahydroimidazo[1,2-a]pyridin-2-yl)-2-hydroxypropanamide (200 mg, 49% yield) as a yellow solid.

LCMS (ESI) [M+H]⁺: 322.

### Step 6: (2R)-1-((5-(3,5-difluorophenyl)-5,6,7,8-tetrahydroimidazo[1,2-a]pyridin-2-yl)amino)-1-oxopropan-2-yl 2-nitrobenzenesulfonate

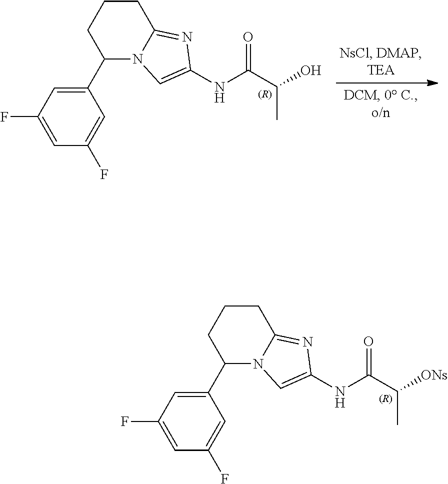

A solution of (2R)—N-(5-(3,5-difluorophenyl)-5,6,7,8-tetrahydroimidazo[1,2-a]pyridin-2-yl)-2-hydroxypropanamide (200 mg, 0.6 mmol), DMAP (7 mg, 60.0 μmol), TEA (126 mg, 1.2 mmol) and 2-nitrobenzene-1-sulfonyl chloride (152 mg, 0.7 mmol) in DCM (8 mL) was stirred at 0° C. overnight under nitrogen atmosphere. The reaction was quenched with water (10 mL) at 0° C. The aqueous layer was extracted with CH₂Cl₂ (3×12 mL). The combined organic layers were dried over anhydrous Na₂SO₄, filtered and concentrated under reduced pressure. The crude residue was purified by silica gel column chromatography, eluted with PE/EA (1:1) to afford (2R)-1-((5-(3,5-difluorophenyl)-5,6,7,8-tetrahydroimidazo[1,2-a]pyridin-2-yl)amino)-1-oxopropan-2-yl 2-nitrobenzenesulfonate (200 mg, 33% yield) as a yellow solid.

LCMS (ESI) [M+H]⁺: 507.

### Step 7: (2S)-2-((S)-4,4-difluoro-3-(6-oxo-1,6-dihydropyridin-3-yl)piperidin-1-yl)-N-(5-(3,5-difluorophenyl)-5,6,7,8-tetrahydroimidazo[1,2-a]pyridin-2-yl)propanamide

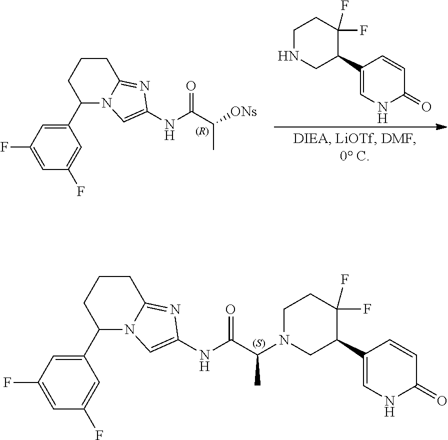

A solution of (2R)-1-((5-(3,5-difluorophenyl)-5,6,7,8-tetrahydroimidazo[1,2-a]pyridin-2-yl)amino)-1-oxopropan-2-yl 2-nitrobenzenesulfonate (70 mg, 0.1 mmol), (S)-5-(4,4-difluoropiperidin-3-yl)pyridin-2(1H)-one (36 mg, 0.2 mmol), DIEA (45 mg, 0.4 mmol) and lithium trifluoromethanesulfonate (54 mg, 0.4 mmol) in DMF (3 mL) was stirred at 0° C. overnight under nitrogen atmosphere. The resulting mixture was concentrated under reduced pressure. The crude residue was purified by reversed-phase flash chromatography (conditions: column, C18 silica gel; mobile phase, ACN in water (0.1% formic acid), 0% to 100% gradient in 25 min; detector, UV 254 nm) to give (2S)-2-((S)-4,4-difluoro-3-(6-oxo-1,6-dihydropyridin-3-yl)piperidin-1-yl)-N-(5-(3,5-difluorophenyl)-5,6,7,8-tetrahydroimidazo[1,2-a]pyridin-2-yl)propanamide (50 mg, 65% yield) as a colorless oil.

LCMS (ESI) [M+H]⁺: 518.

### Step 8: (S)-2-((S)-4,4-difluoro-3-(6-oxo-1,6-dihydropyridin-3-yl)piperidin-1-yl)-N—((R)-5-(3,5-difluorophenyl)-5,6,7,8-tetrahydroimidazo[1,2-a]pyridin-2-yl)propanamide and (S)-2-((S)-4,4-difluoro-3-(6-oxo-1,6-dihydropyridin-3-yl)piperidin-1-yl)-N—((S)-5-(3,5-difluorophenyl)-5,6,7,8-tetrahydroimidazo[1,2-a]pyridin-2-yl)propanamide

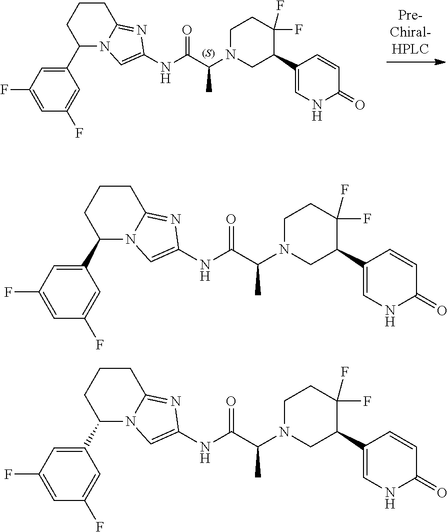

(2S)-2-((S)-4,4-difluoro-3-(6-oxo-1,6-dihydropyridin-3-yl)piperidin-1-yl)-N-(5-(3,5-difluorophenyl)-5,6,7,8-tetrahydroimidazo[1,2-a]pyridin-2-yl)propanamide (45 mg) was separated by Prep-Chiral HPLC with the following conditions (Column: Lux 5 μm Cellulose-4 3*25 cm, 5 μm; Mobile Phase A: Hex (10 mM NH₃-MeOH), Mobile Phase B: EtOH:ACN=5:1; Flow rate: 40 mL/min; Gradient: isocratic 50% B; Wave Length: 232/254 nm; RT1 (min): 6.032; RT2 (min): 8.17) to afford first peak (S)-2-((S)-4,4-difluoro-3-(6-oxo-1,6-dihydropyridin-3-yl)piperidin-1-yl)-N—((R)-5-(3,5-difluorophenyl)-5,6,7,8-tetrahydroimidazo[1,2-a]pyridin-2-yl)propanamide (Method Y, Peak 2, 1.48 min; 13.7 mg, 100% d.e. Compound 507) as a white solid and second peak (S)-2-((S)-4,4-difluoro-3-(6-oxo-1,6-dihydropyridin-3-yl)piperidin-1-yl)-N—((S)-5-(3,5-difluorophenyl)-5,6,7,8-tetrahydroimidazo[1,2-a]pyridin-2-yl)propanamide (Method Y, Peak 1, 1.27 min; 15.5 mg, 100% d.e. Compound 508) as a white solid.

TABLE 35

| Compound# | Characterization Data |
|---|---|
| 507 | LCMS (ESI) [M + H]⁺: 518.2 |
| | ¹H NMR (400 MHz, DMSO-d₆) δ 11.55 (bs, 1H), 10.10 (s, 1H), 7.41-7.35 (m, 1H), 7.29- |
| | 7.13 (m, 2H), 6.95-6.84 (m, 2H), 6.71 (s, 1H), 6.28 (d, J = 9.5 Hz, 1H), 5.46-5.08 (m, 1H), |
| | 3.49 (d, J = 6.8 Hz, 1H), 3.21-3.06 (m, 1H), 2.90-2.64 (m, 5H), 2.48-2.40 (m, 1H), 2.29- |
| | 2.20 (m, 1H), 2.12-1.92 (m, 3H), 1.87-1.71 (m, 2H), 1.13 (d, J = 6.9 Hz, 3H). |
| 508 | LCMS (ESI) [M + H]⁺: 518.3 |
| | ¹H NMR (400 MHz, DMSO-d₆) δ 11.54 (bs, 1H), 10.01 (s, 1H), 7.38 (d, J = 10.0 Hz, 1H), |
| | 7.32-7.17 (m, 2H), 7.00-6.90 (m, 2H), 6.68 (s, 1H), 6.28 (d, J = 9.4 Hz, 1H), 5.37-5.22 |
| | (m, 1H), 3.52-3.54 (m, 1H), 3.28-3.10 (m, 1H), 2.91-2.66 (m, 5H), 2.41 (dd, J = 11.8 Hz, |
| | 1H), 2.28-2.19 (m, 1H), 2.12-1.94 (m, 3H), 1.93-1.69 (m, 2H), 1.13 (d, J = 6.9 Hz, 3H). |

### Example 62: (2S)—N-(5-(3,5-difluorophenyl)-6,7-dihydro-5H-pyrrolo[1,2-a]imidazol-2-yl)-2-(4,4-difluoropiperidin-1-yl)propanamide

### Step 1: (2S)—N-(5-(3,5-difluorophenyl)-6,7-dihydro-5H-pyrrolo[1,2-a]imidazol-2-yl)-2-(4,4-difluoropiperidin-1-yl)propanamide

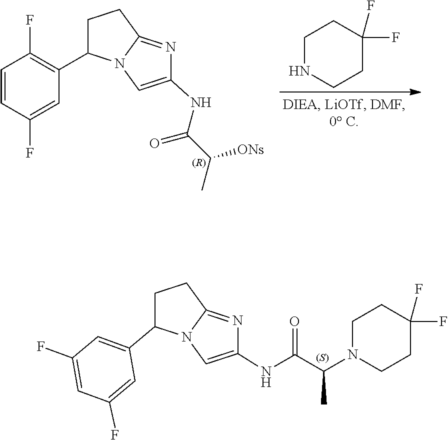

To a mixture of 4,4-difluoropiperidine (242 mg, 2.0 mmol) in N,N-dimethylacetamide (5 mL) were added DIEA (387 mg, 3.0 mmol), lithium trifluoromethanesulfonate (499 mg, 3.2 mmol) and (2R)-1-((5-(3,5-difluorophenyl)-6,7-dihydro-5H-pyrrolo[1,2-a]imidazol-2-yl)amino)-1-oxopropan-2-yl 2-nitrobenzenesulfonate (500 mg, 1.0 mmol) at 0° C. under nitrogen atmosphere. The resulting mixture was stirred at 0° C. overnight under nitrogen atmosphere. The resulting mixture was concentrated under reduced pressure. The crude reaction mixture was purified by reverse phase flash chromatography (conditions: column, C18 silica gel; mobile phase, ACN in water (0.1% formic acid), 0% to 100% gradient in 25 min; detector, UV 254 nm and UV 220 nm) to give (2S)—N-(5-(3,5-difluorophenyl)-6,7-dihydro-5H-pyrrolo[1,2-a]imidazol-2-yl)-2-(4,4-difluoropiperidin-1-yl)propanamide (334 mg, 80% yield) as a colorless oil.

LCMS (ESI) [M+H]⁺: 411.

### Step 2: (S)—N—((R)-5-(3,5-difluorophenyl)-6,7-dihydro-5H-pyrrolo[1,2-a]imidazol-2-yl)-2-(4,4-difluoropiperidin-1-yl)propanamide and (S)—N—((S)-5-(3,5-difluorophenyl)-6,7-dihydro-5H-pyrrolo[1,2-a]imidazol-2-yl)-2-(4,4-difluoropiperidin-1-yl)propanamide

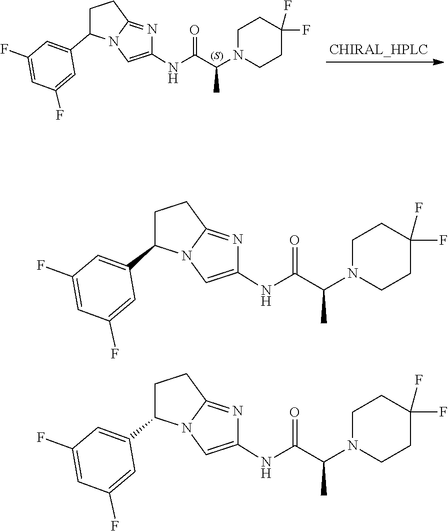

(2S)—N-(5-(3,5-difluorophenyl)-6,7-dihydro-5H-pyrrolo[1,2-a]imidazol-2-yl)-2-(4,4-difluoropiperidin-1-yl)propanamide (142 mg) was purified by Prep-Chiral HPLC with the following conditions (Column: CHIRALPAK-IK, 3*25 cm, 5 μm; Mobile Phase A: Hex (10 mM NH₃-MeOH), Mobile Phase B: EtOH; Flow rate: 40 mL/min; Gradient: isocratic 10% B; Wave Length: 216/225 nm; RT1 (min): 16.4; RT2 (min): 25.8) to afford first peak (S)—N—((R)-5-(3,5-difluorophenyl)-6,7-dihydro-5H-pyrrolo[1,2-a]imidazol-2-yl)-2-(4,4-difluoropiperidin-1-yl)propanamide (Method AB, Peak 1, 1.70 min; 43.5 mg, 100% d.e. Compound 509) as a white solid and second peak (S)—N—((S)-5-(3,5-difluorophenyl)-6,7-dihydro-5H-pyrrolo[1,2-a]imidazol-2-yl)-2-(4,4-difluoropiperidin-1-yl)propanamide (Method AB, Peak 2, 2.61 min; 42.8 mg, 100% d.e. Compound 510) as a white solid.

TABLE 36

| Compound# | Characterization Data |
|---|---|
| 509 | LCMS (ESI) [M + H]⁺: 411.2 |
| | ¹H NMR (400 MHz, DMSO-d₆) δ 10.03 (s, 1H), 7.22 (m, 1H), 7.00-6.83 (m, 3H), 5.38 (dd, |
| | J = 7.9, 5.3 Hz, 1H), 3.48-3.41 (m, 1H), 3.04-2.86 (m, 2H), 2.82-2.71 (m, 1H), 2.69- |
| | 2.50 (m, 4H), 2.41-2.32 (m, 1H), 2.04-1.86 (m, 4H), 1.13 (d, J = 6.9 Hz, 3H). |
| 510 | LCMS (ESI) [M + H]⁺: 411.2 |
| | ¹H NMR (400 MHz, DMSO-d₆) δ 10.09 (s, 1H), 7.28-7.19 (m, 1H), 6.98 (s, 1H), 6.96-6.83 |
| | (m, 2H), 5.39 (dd, J = 8.2, 5.2 Hz, 1H), 3.49-3.42 (m, 1H), 3.03-2.83 (m, 2H), 2.83-2.72 |
| | (m, 1H), 2.70-2.50 (m, 4H), 2.42-2.30 (m, 1H), 2.03-1.85 (m, 4H), 1.13 (d, J = 6.9 Hz, |
| | 3H). |

### Example 63: N-(5-(3,5-difluorophenyl)-6,7-dihydro-5H-pyrrolo[1,2-a]imidazol-2-yl)-2-morpholinopropanamide

### Step 1: N-(5-(3,5-difluorophenyl)-6,7-dihydro-5H-pyrrolo[1,2-a]imidazol-2-yl)-2-morpholinopropanamide

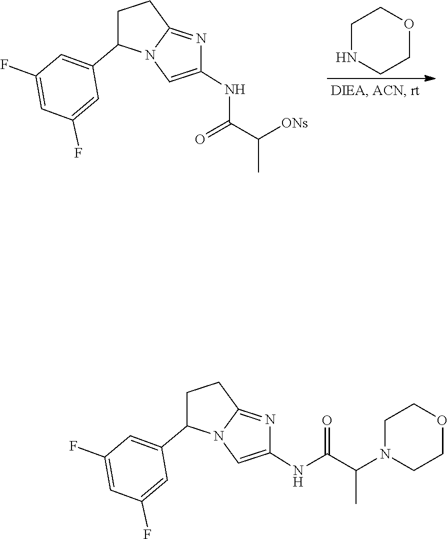

To a mixture of morpholine (42 mg, 483 μmol) in ACN (8 mL) was added 1-((5-(3,5-difluorophenyl)-6,7-dihydro-5H-pyrrolo[1,2-a]imidazol-2-yl)amino)-1-oxopropan-2-yl 2-nitrobenzenesulfonate (237 mg, 483 μmol) and DIEA (187 mg, 1.4 mmol) dropwise at room temperature under nitrogen atmosphere. The resulting mixture was stirred at room temperature for 3 h under nitrogen atmosphere. The resulting mixture was concentrated under reduced pressure and the crude residue was directly purified by reversed-phase flash chromatography (conditions: column, C18 silica gel; mobile phase, MeCN in water (10 mmol/L NH₄HCO₃), 0% to 100% gradient in 30 min; detector, UV 254 nm) to give N-(5-(3,5-difluorophenyl)-6,7-dihydro-5H-pyrrolo[1,2-a]imidazol-2-yl)-2-morpholinopropanamide (120 mg, 65% yield) as a light yellow solid.

LCMS (ESI) [M+H]⁺: 377.

### Step 2: (S)—N—((R)-5-(3,5-difluorophenyl)-6,7-dihydro-5H-pyrrolo[1,2-a]imidazol-2-yl)-2-morpholinopropanamide and (R)—N—((R)-5-(3,5-difluorophenyl)-6,7-dihydro-5H-pyrrolo[1,2-a]imidazol-2-yl)-2-morpholinopropanamide and (R)—N—((S)-5-(3,5-difluorophenyl)-6,7-dihydro-5H-pyrrolo[1,2-a]imidazol-2-yl)-2-morpholinopropanamide and (S)—N—((S)-5-(3,5-difluorophenyl)-6,7-dihydro-5H-pyrrolo[1,2-a]imidazol-2-yl)-2-morpholinopropanamide

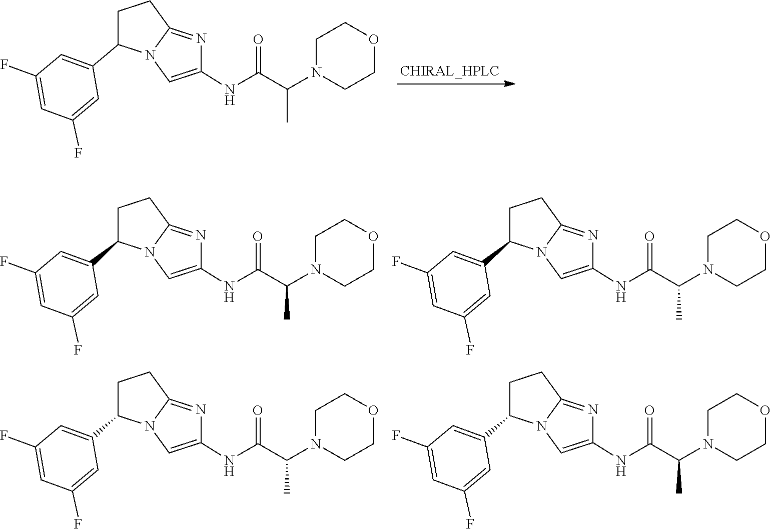

N-(5-(3,5-difluorophenyl)-6,7-dihydro-5H-pyrrolo[1,2-a]imidazol-2-yl)-2-morpholinopropanamide (120 mg) was purified by Prep-Chiral HPLC with the following conditions (Column: Lux 5 μm Cellulose-2 3*15 cm, 5 μm; Mobile Phase A: Hex (10 mM NH₃-MeOH), Mobile Phase B: EtOH:ACN=5:1; Flow rate: 40 mL/min; Gradient: isocratic 50% B; Wave Length: 210/242 nm) to afford two portions (45 mg, RT1 (min): 6.8, 65 mg, RT2 (min): 11.2), each as a mixture of diastereomers. The first portion was further separated by Prep-Chiral HPLC with the following conditions (Column: Lux 5 μm Cellulose-2, 30*250 mm, 5.0 μm; Mobile Phase A: Hex (10 mM NH₃-MeOH), Mobile Phase B: EtOH:ACN=5:1; Flow rate: 40 mL/min; Gradient: isocratic 50% B; Wave Length: 220/270 nm; RT1 (min): 6.07; RT2 (min): 7.064) to afford first peak (S)—N—((R)-5-(3,5-difluorophenyl)-6,7-dihydro-5H-pyrrolo[1,2-a]imidazol-2-yl)-2-morpholinopropanamide (Method AC, Peak 1, 3.10 min; 8.4 mg, 100% d.e. Compound 511) as a white solid and second peak (R)—N—((R)-5-(3,5-difluorophenyl)-6,7-dihydro-5H-pyrrolo[1,2-a]imidazol-2-yl)-2-morpholinopropanamide (Method AC, Peak 2, 3.51 min; 19.6 mg, 100% d.e. Compound 512) as a white solid.

The second portion was further separated by Prep-Chiral HPLC with the following conditions (Column: CHIRALPAK-IK, 3*25 cm, 5 μm; Mobile Phase A: Hex (10 mM NH₃-MeOH), Mobile Phase B: EtOH; Flow rate: 40 mL/min; Gradient: isocratic 10% B; Wave Length: 220/254 nm; RT1 (min): 25.64; RT2 (min): 30.04) to afford first peak (R)—N—((S)-5-(3,5-difluorophenyl)-6,7-dihydro-5H-pyrrolo[1,2-a]imidazol-2-yl)-2-morpholinopropanamide (Method AD, Peak 1, 1.68 min; 10.9 mg, 100% d.e. Compound 513) as a white solid and second peak (S)—N—((S)-5-(3,5-difluorophenyl)-6,7-dihydro-5H-pyrrolo[1,2-a]imidazol-2-yl)-2-morpholinopropanamide (Method AD, Peak 2, 2.84 min; 25.3 mg, 99% d.e. Compound 514) as a white solid.

TABLE 37

| Compound# | Characterization Data |
|---|---|
| 511 | LCMS (ESI) [M + H]⁺: 377.2 |
| | ¹H NMR (400 MHz, DMSO-d₆) δ 9.99 (s, 1H), 7.28-7.18 (m, 1H), 6.98 (s, 1H), 6.95-6.87 |
| | (m, 2H), 5.54-5.46 (m, 1H), 3.57 (dd, J = 4.6 Hz, 4H), 3.27 (d, J = 6.9 Hz, 1H), 3.04-2.87 |
| | (m, 2H), 2.83-2.73 (m, 1H), 2.54-2.30 (m, 5H), 1.12 (d, J = 6.9 Hz, 3H). |
| 512 | LCMS (ESI) [M + H]⁺: 377.2 |
| | ¹H NMR (400 MHz, DMSO-d₆) δ 9.94 (d, J = 1.8 Hz, 1H), 7.28-7.19 (m, 1H), 7.01-6.85 |
| | (m, 3H), 5.44-5.35 (m, 1H), 3.57 (dd, J = 4.6 Hz, 4H), 3.26 (d, J = 6.9 Hz, 1H), 3.04-2.88 |
| | (m, 2H), 2.85-2.74 (m, 1H), 2.56-2.32 (m, 5H), 1.12 (d, J = 6.9 Hz, 3H). |
| 513 | LCMS (ESI) [M + H]⁺: 377.2 |
| | ¹H NMR (400 MHz, DMSO-d₆) δ 9.99 (s, 1H), 7.28-7.18 (m, 1H), 6.98 (s, 1H), 6.96-6.84 |
| | (m, 2H), 5.44-5.36 (m, 1H), 3.57 (dd, J = 4.6 Hz, 4H), 3.27 (d, J = 6.9 Hz, 1H), 3.03-2.87 |
| | (m, 2H), 2.86-2.70 (m, 1H), 2.55-2.27 (m, 5H), 1.12 (d, J = 6.9 Hz, 3H). |
| 514 | LCMS (ESI) [M + H]⁺: 377.2 |
| | ¹H NMR (400 MHz, DMSO-d₆) δ 9.94 (bs, 1H), 7.26-7.19 (m, 1H), 7.01-6.85 (m, 3H), |
| | 5.44-5.37 (m, 1H), 3.60 (bs 4H), 3.30-3.05 (m, 1H), 3.03-2.74 (m, 3H), 2.60- 2.32 (m, |
| | 5H), 1.12 (d, J = 6.9 Hz, 3H). |

### Example 64: (2S)-2-((S)-4,4-difluoro-3-(6-oxo-1,6-dihydropyridin-3-yl)piperidin-1-yl)-N-(5-(3,5-difluorophenyl)-7-hydroxy-6,7-dihydro-5H-pyrrolo[1,2-a]imidazol-2-yl)propanamide

### Step 1: (E)-3-(3,5-difluorophenyl)acrylaldehyde

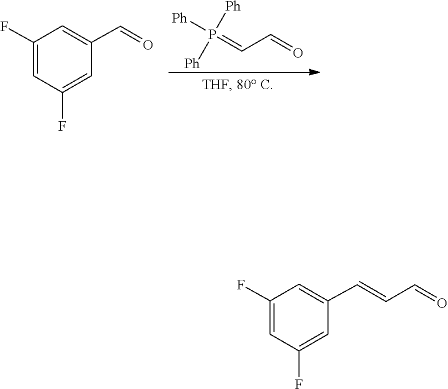

A solution of 3,5-difluorobenzaldehyde (30.00 g, 211.1 mmol) and 2-(triphenyl-15-phosphaneylidene)acetaldehyde (77.10 g, 253.3 mmol) in THF (500 mL) was stirred at 80° C. for 16 h under nitrogen atmosphere. The mixture was allowed to cool down to room temperature. The resulting mixture was concentrated under reduced pressure. The crude residue was directly purified by silica gel column chromatography, eluted with PE/EA (3:1) to afford (E)-3-(3,5-difluorophenyl)acrylaldehyde (30.00 g, 85% yield) as a yellow solid.

LCMS (ESI) [M+H]⁺: 169.

### Step 2: 5-(3,5-difluorophenyl)-6,7-dihydro-5H-pyrrolo[1,2-a]imidazol-7-ol

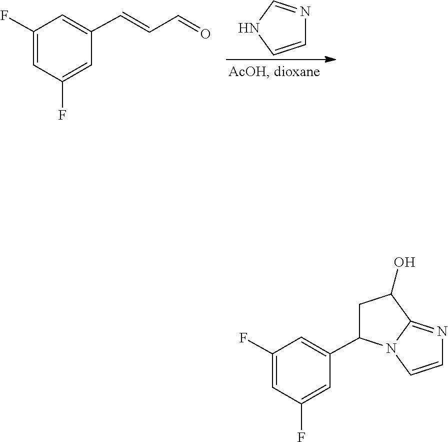 [SMILES: O=C/C=C/c1cc(F)cc(F)c1 ; c1c[nH]cn1 ; OC1CC(c2cc(F)cc(F)c2)n2ccnc21]

A solution of (E)-3-(3,5-difluorophenyl)acrylaldehyde (30.00 g, 178.4 mmol), AcOH (5.35 g, 89.2 mmol) and imidazole (36.44 g, 535.3 mmol) in dioxane (500 mL) was stirred at 100° C. for 72 h under nitrogen atmosphere. The mixture was allowed to cool down to room temperature. The resulting mixture was concentrated under reduced pressure. The residue was directly purified by reversed-phase flash chromatography (conditions: column, C18 silica gel; mobile phase, MeCN in water (0.1% formic acid), 0% to 100% gradient in 20 min; detector, UV 254 nm) to give 5-(3,5-difluorophenyl)-6,7-dihydro-5H-pyrrolo[1,2-a]imidazol-7-ol (10.00 g, 24% yield) as a yellow solid.

LCMS (ESI) [M+H]⁺: 237.

### Step 3: 7-((tert-butyldiphenylsilyl)oxy)-5-(3,5-difluorophenyl)-6,7-dihydro-5H-pyrrolo[1,2-a]imidazole

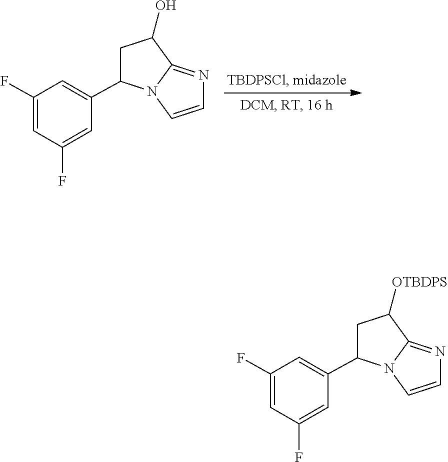

A solution of 5-(3,5-difluorophenyl)-6,7-dihydro-5H-pyrrolo[1,2-a]imidazol-7-ol (10.00 g, 42.3 mmol) and tert-butyl(chloro)diphenylsilane (17.45 g, 63.5 mmol), imidazole (8.64 g, 127.1 mmol) in DCM (150 mL) was stirred at room temperature for 16 h under nitrogen atmosphere. The reaction was quenched with water (200 mL) at 0° C. The resulting mixture was extracted with DCM (3×250 mL). The combined organic layers were dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 0% to 100% gradient in 20 min; detector, UV 254 nm) to give 7-((tert-butyldiphenylsilyl)oxy)-5-(3,5-difluorophenyl)-6,7-dihydro-5H-pyrrolo[1,2-a]imidazole (11.00 g, 55% yield) as a yellow oil.

LCMS (ESI) [M+H]⁺: 475.

### Step 4: 2,3-dibromo-7-((tert-butyldiphenylsilyl)oxy)-5-(3,5-difluorophenyl)-6,7-dihydro-5H-pyrrolo[1,2-a]imidazole

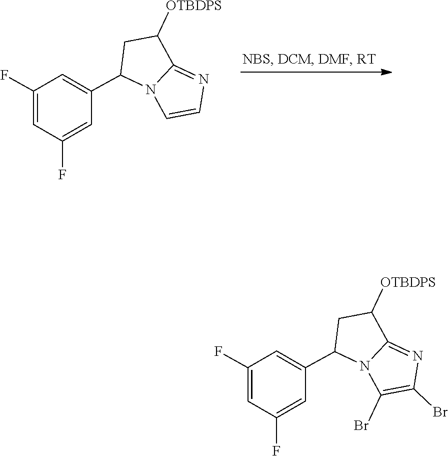

To a mixture of 7-((tert-butyldiphenylsilyl)oxy)-5-(3,5-difluorophenyl)-6,7-dihydro-5H-pyrrolo[1,2-a]imidazole (11.00 g, 23.2 mmol) in DCM (55 mL) were added DMF (55 mL) and NBS (9.07 g, 51.0 mmol) at 0° C. The resulting mixture was stirred at room temperature for 3 h under nitrogen atmosphere. The reaction was diluted with water (150 mL) at 0° C. The resulting mixture was extracted with DCM (3×200 mL). The combined organic layers were dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 0% to 100% gradient in 20 min; detector, UV 254 nm) to give 2,3-dibromo-7-((tert-butyldiphenylsilyl)oxy)-5-(3,5-difluorophenyl)-6,7-dihydro-5H-pyrrolo[1,2-a]imidazole (12.00 g, 82% yield) as a yellow solid.

LCMS (ESI) [M+H]⁺: 631.

### Step 5: 2-bromo-7-((tert-butyldiphenylsilyl)oxy)-5-(3,5-difluorophenyl)-6,7-dihydro-5H-pyrrolo[1,2-a]imidazole

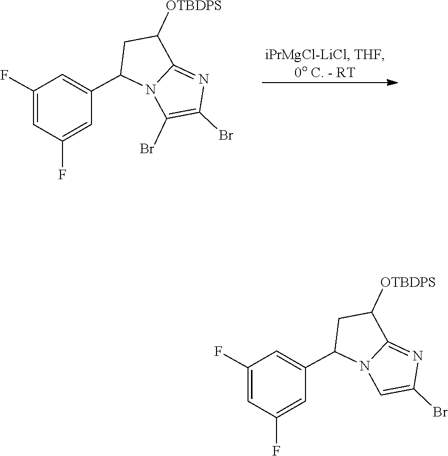

To a mixture of 2,3-dibromo-7-((tert-butyldiphenylsilyl)oxy)-5-(3,5-difluorophenyl)-6,7-dihydro-5H-pyrrolo[1,2-a]imidazole (12.00 g, 19.0 mmol) in THF (150 mL) was added isopropyl magnesium chloride-lithium chloride complex (17.5 mL, 22.8 mmol, 1.3 M in THF) dropwise over 8 min at 0° C. The resulting mixture was stirred at room temperature for 3 h under nitrogen atmosphere. The reaction mixture was quenched with NH₄Cl solution (180 mL) at 0° C. The aqueous layer was extracted with CH₂Cl₂ (3×200 mL). The combined organic layers were dried over anhydrous Na₂SO₄, filtered and concentrated under reduced pressure. The crude residue was purified by silica gel column chromatography, eluted with PE/EA (5:1) to afford 2-bromo-7-((tert-butyldiphenylsilyl)oxy)-5-(3,5-difluorophenyl)-6,7-dihydro-5H-pyrrolo[1,2-a]imidazole (9.00 g, 86% yield) as a yellow oil.

LCMS (ESI) [M+H]⁺: 553.

### Step 6: (2R)—N-(7-((tert-butyldiphenylsilyl)oxy)-5-(3,5-difluorophenyl)-6,7-dihydro-5H-pyrrolo[1,2-a]imidazol-2-yl)-2-hydroxypropanamide

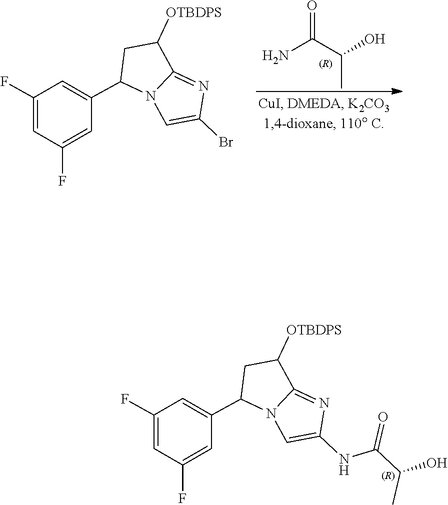

A solution of 2-bromo-7-((tert-butyldiphenylsilyl)oxy)-5-(3,5-difluorophenyl)-6,7-dihydro-5H-pyrrolo[1,2-a]imidazole (3.00 g, 5.4 mmol), (R)-2-hydroxypropanamide (965 mg, 10.8 mmol), CuI (206 mg, 1.1 mmol), K₂CO₃ (1.50 g, 10.8 mmol) and 1,2-bis(dimethylamino)ethane (191 mg, 2.2 mmol) in 1,4-dioxane (35 mL) was stirred at 110° C. overnight under nitrogen atmosphere. The mixture was allowed to cool down to room temperature. The reaction was diluted with water at 0° C. The aqueous layer was extracted with EtOAc (3×40 mL). The combined organic layers were dried over anhydrous Na₂SO₄, filtered and concentrated under reduced pressure. The crude residue was purified by silica gel column chromatography, eluted with PE/EA (1:1) to afford (2R)—N-(7-((tert-butyldiphenylsilyl)oxy)-5-(3,5-difluorophenyl)-6,7-dihydro-5H-pyrrolo[1,2-a]imidazol-2-yl)-2-hydroxypropanamide (2.50 g, 82% yield) as a yellow solid.

LCMS (ESI) [M+H]⁺: 562.

### Step 7: (2R)-1-((7-((tert-butyldiphenylsilyl)oxy)-5-(3,5-difluorophenyl)-6,7-dihydro-5H-pyrrolo[1,2-a]imidazol-2-yl)amino)-1-oxopropan-2-yl 2-nitrobenzenesulfonate

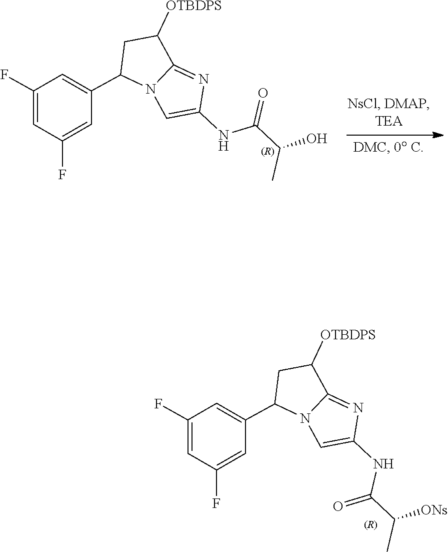

A solution of (2R)—N-(7-((tert-butyldiphenylsilyl)oxy)-5-(3,5-difluorophenyl)-6,7-dihydro-5H-pyrrolo[1,2-a]imidazol-2-yl)-2-hydroxypropanamide (1.80 g, 3.2 mmol), DMAP (78 mg, 0.6 mmol), TEA (648 mg, 6.4 mmol) and 2-nitrobenzene-1-sulfonyl chloride (1.42 g, 6.4 mmol) in DCM (20 mL) was stirred at 0° C. overnight under nitrogen atmosphere. The reaction was quenched with water (25 mL) at 0° C. The aqueous layer was extracted with CH₂Cl₂ (3×30 mL). The combined organic layers were dried over anhydrous Na₂SO₄, filtered and concentrated under reduced pressure. The crude residue was purified by silica gel column chromatography, eluted with CH₂Cl₂/EA (1:2) to afford (2R)-1-((7-((tert-butyldiphenylsilyl)oxy)-5-(3,5-difluorophenyl)-6,7-dihydro-5H-pyrrolo[1,2-a]imidazol-2-yl)amino)-1-oxopropan-2-yl 2-nitrobenzenesulfonate (1.45 g, 61% yield) as a yellow solid.

LCMS (ESI) [M+H]⁺: 747.

### Step 8: (2S)—N-(7-((tert-butyldiphenylsilyl)oxy)-5-(3,5-difluorophenyl)-6,7-dihydro-5H-pyrrolo[1,2-a]imidazol-2-yl)-2-((S)-4,4-difluoro-3-(6-oxo-1,6-dihydropyridin-3-yl)piperidin-1-yl)propanamide

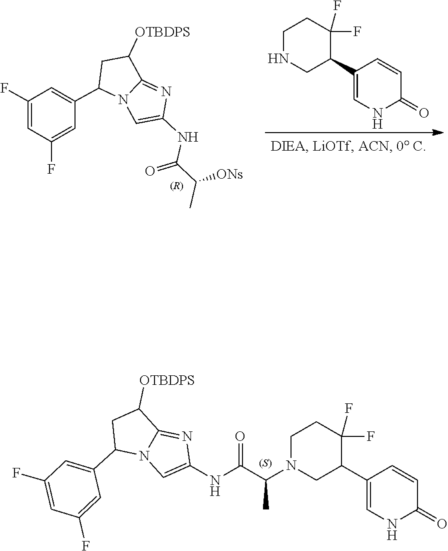

A solution of (2R)-1-((7-((tert-butyldiphenylsilyl)oxy)-5-(3,5-difluorophenyl)-6,7-dihydro-5H-pyrrolo[1,2-a]imidazol-2-yl)amino)-1-oxopropan-2-yl 2-nitrobenzenesulfonate (1.40 g, 1.9 mmol), (S)-5-(4,4-difluoropiperidin-3-yl)pyridin-2(1H)-one (481 mg, 2.3 mmol), DIEA (726 mg, 5.6 mmol) and lithium trifluoromethanesulfonate (877 mg, 5.6 mmol) in ACN (15 mL) was stirred at 0° C. for 4 days under nitrogen atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 25 min; detector, UV 254 nm) to give (2S)—N-(7-((tert-butyldiphenylsilyl)oxy)-5-(3,5-difluorophenyl)-6,7-dihydro-5H-pyrrolo[1,2-a]imidazol-2-yl)-2-((S)-4,4-difluoro-3-(6-oxo-1,6-dihydropyridin-3-yl)piperidin-1-yl)propanamide (750 mg, 53% yield) as a white solid.

LCMS (ESI) [M+H]⁺: 758.

### Step 9: (2S)-2-((S)-4,4-difluoro-3-(6-oxo-1,6-dihydropyridin-3-yl)piperidin-1-yl)-N-(5-(3,5-difluorophenyl)-7-hydroxy-6,7-dihydro-5H-pyrrolo[1,2-a]imidazol-2-yl)propanamide

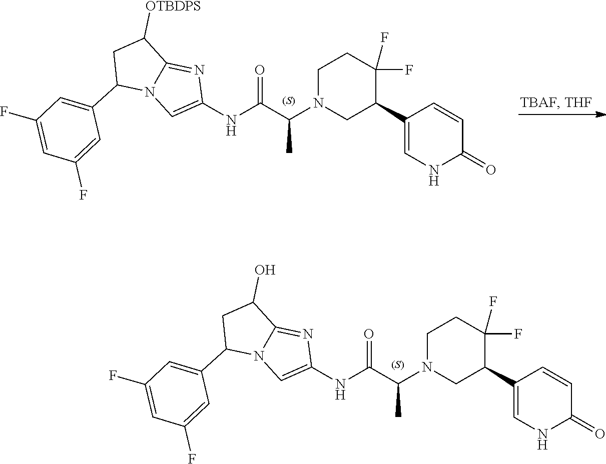

To a mixture of (2S)—N-(7-((tert-butyldiphenylsilyl)oxy)-5-(3,5-difluorophenyl)-6,7-dihydro-5H-pyrrolo[1,2-a]imidazol-2-yl)-2-((S)-4,4-difluoro-3-(6-oxo-1,6-dihydropyridin-3-yl)piperidin-1-yl)propanamide (500 mg, 0.7 mmol) in THF (8 mL) was added TBAF (0.8 mL, 0.8 mmol, 1M in THF) dropwise at 0° C. under nitrogen atmosphere. The resulting mixture was stirred at room temperature for 16 h under nitrogen atmosphere. The reaction was quenched with water (10 mL) at 0° C. The aqueous layer was extracted with CH₂Cl₂ (3×15 mL). The combined organic layers were dried over anhydrous Na₂SO₄, filtered and concentrated under reduced pressure. The crude residue was purified by silica gel column chromatography, eluted with PE/EA (1:1) to afford (2S)-2-((S)-4,4-difluoro-3-(6-oxo-1,6-dihydropyridin-3-yl)piperidin-1-yl)-N-(5-(3,5-difluorophenyl)-7-hydroxy-6,7-dihydro-5H-pyrrolo[1,2-a]imidazol-2-yl)propanamide (214 mg, 62% yield) as a white solid.

LCMS (ESI) [M+H]⁺: 520.

### Step 10: (S)-2-((S)-4,4-difluoro-3-(6-oxo-1,6-dihydropyridin-3-yl)piperidin-1-yl)-N-((5R,7R)-5-(3,5-difluorophenyl)-7-hydroxy-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N-((5R,7S)-5-(3,5-difluorophenyl)-7-hydroxy-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N-((5S,7S)-5-(3,5-difluorophenyl)-7-hydroxy-6,7-dihydro-5H-pyrrolo[1,2-a]imidazol-2-yl)propanamide and (S)-2-((S)-4,4-difluoro-3-(6-oxo-1,6-dihydropyridin-3-yl)piperidin-1-yl)-N-((5S,7R)-5-(3,5-difluorophenyl)-7-hydroxy-6,7-dihydro-5H-pyrrolo[1,2-a]imidazol-2-yl)propanamide

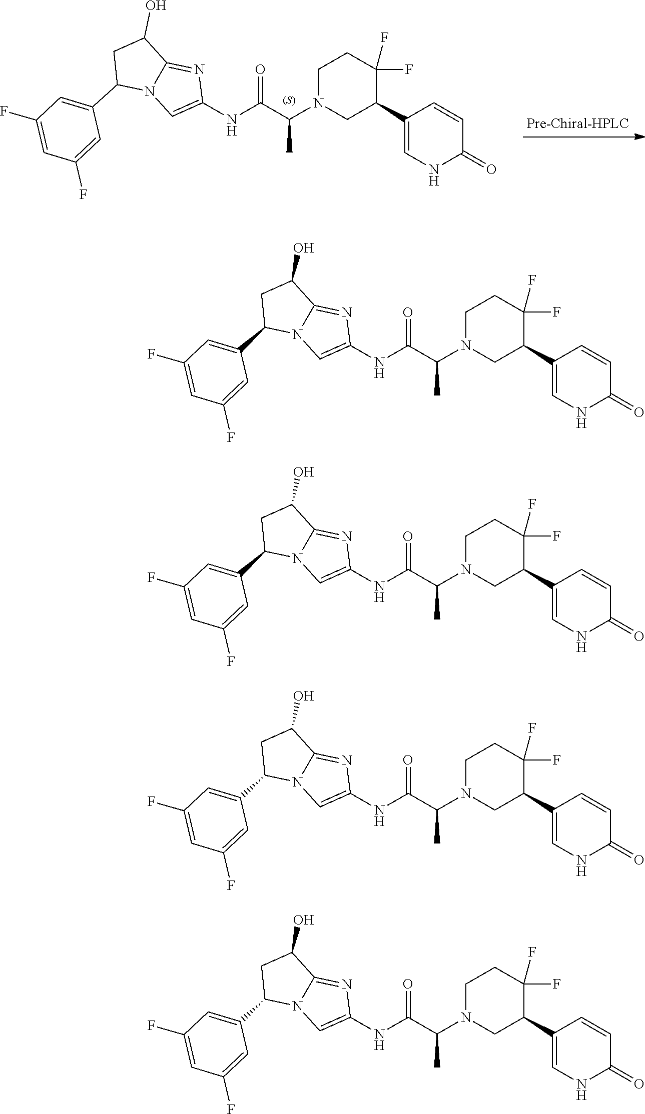

(2S)-2-((S)-4,4-difluoro-3-(6-oxo-1,6-dihydropyridin-3-yl)piperidin-1-yl)-N-(5-(3,5-difluorophenyl)-7-hydroxy-6,7-dihydro-5H-pyrrolo[1,2-a]imidazol-2-yl)propanamide (62 mg) was separated by Prep-Chiral HPLC with the following conditions (Column: Lux 5 μm Cellulose-2, 30*250 mm, 5.0 μm; Mobile Phase A: Hex (10 mM NH₃-MeOH), Mobile Phase B: EtOH; Flow rate: 40 mL/min; Gradient: isocratic 30% B; Wave Length: 230/240 nm; RT1 (min): 16.5; RT2 (min): 27), each is a mixture of diastereomers. The first portion was further separated by Prep-Chiral HPLC with the following conditions (Column: CHIRALPAK IA, 3×25 cm, 5 μm; Mobile Phase A: MtBE (10 mM NH₃-MeOH), Mobile Phase B: EtOH; Flow rate: 40 mL/min; Gradient: isocratic 50% B; Wave Length: 212/268 nm; RT1 (min): 5.89; RT2 (min): 10.08) to afford first peak (S)-2-((S)-4,4-difluoro-3-(6-oxo-1,6-dihydropyridin-3-yl)piperidin-1-yl)-N-((5R,7R)-5-(3,5-difluorophenyl)-7-hydroxy-6,7-dihydro-5H-pyrrolo[1,2-a]imidazol-2-yl)propanamide (Method AE, Peak 1, 0.88 min; 9.0 mg, 100% d.e.) (Compound 515) as a white solid and second peak (S)-2-((S)-4,4-difluoro-3-(6-oxo-1,6-dihydropyridin-3-yl)piperidin-1-yl)-N-((5R,7S)-5-(3,5-difluorophenyl)-7-hydroxy-6,7-dihydro-5H-pyrrolo[1,2-a]imidazol-2-yl)propanamide (Method AE, Peak 2, 1.45 min; 8.0 mg, 100% d.e.) (Compound 516) as a white solid.

The second portion was further separated by Prep-Chiral HPLC with the following conditions (Column: CHIRAL ART Cellulose-SC, 3*25 cm, 5 μm; Mobile Phase A: Hex (10 mM NH₃-MeOH), Mobile Phase B: EtOH; Flow rate: 40 mL/min; Gradient: isocratic 20% B; Wave Length: 212/242 nm; RT1 (min): 7.2; RT2 (min): 8.7; RT3 (min): 10.7; RT4 (min): 12.5) to afford first peak (S)-2-((S)-4,4-difluoro-3-(6-oxo-1,6-dihydropyridin-3-yl)piperidin-1-yl)-N-((5S,7S)-5-(3,5-difluorophenyl)-7-hydroxy-6,7-dihydro-5H-pyrrolo[1,2-a]imidazol-2-yl)propanamide (Method AF, Peak 1, 0.94 min; 9.3 mg, 1000 d.e.) (Compound 517) as a white solid and second peak (S)-2-((S)-4,4-difluoro-3-(6-oxo-1,6-dihydropyridin-3-yl)piperidin-1-yl)-N-((5S,7R)-5-(3,5-difluorophenyl)-7-hydroxy-6,7-dihydro-5H-pyrrolo[1,2-a]imidazol-2-yl)propanamide (Method AF, Peak 2, 1.35 min; 13.7 mg, 100) d.e.) (Compound 518) as a white solid.

TABLE 38

| Compound# | Characterization Data |
|---|---|
| 515 | LCMS (ESI) [M + H]⁺: 520.3 |
| | ¹H NMR (400 MHz, DMSO-d₆) δ 11.59 (bs, 1H), 10.16 (bs, 1H), 7.44-7.38 (m, 1H), 7.29 |
| | (d, J = 2.6 Hz, 1H), 7.26-7.19 (m, 1H), 7.16-7.10 (m, 2H), 6.99 (s, 1H), 6.30 (d, J = 9.5 |
| | Hz, 1H), 5.85 (bs, 1H), 5.40-5.30 (m, 1H), 5.00 (d, J = 7.0 Hz, 1H), 3.55 (bs, 1H), 3.33- |
| | 3.26 (m, 1H), 3.26-3.07 (m, 1H), 2.94-2.70 (m, 3H), 2.50-2.39 (m, 1H), 2.18 (dd, J = |
| | 13.9, 3.7 Hz, 1H), 2.15-1.94 (m, 2H), 1.18 (d, J = 6.8 Hz, 3H). |
| 516 | LCMS (ESI) [M + H]⁺: 520.3 |
| | ¹H NMR (400 MHz, DMSO-d₆) δ 11.55 (bs, 1H), 10.14 (s, 1H), 7.40 (d, J = 9.6 Hz, 1H), 7.28 |
| | (s, 1H), 7.26-7.16 (m, 1H), 7.02-6.98 (m, 2H), 6.93 (d, J = 2.6 Hz, 1H), 6.40-6.16 (m, |
| | 1H), 5.68 (dd, J = 6.2, 2.4 Hz, 1H), 5.55-5.49 (m, 1H), 5.08-5.02 (m, 1H), 3.56-3.49 (m, |
| | 1H), 3.25-3.06 (m, 1H), 2.90-2.64 (m, 5H), 2.52-2.41 (m, 1H), 2.14-1.92 (m, 2H), 1.18- |
| | 1.14 (m, 3H). |
| 517 | LCMS (ESI) [M + H]⁺: 520.3 |
| | ¹H NMR (400 MHz, DMSO-d₆) δ 11.57 (bs, 1H), 10.21 (s, 1H), 7.42-7.38 (m, 1H), 7.28 (s, |
| | 1H), 7.26-7.19 (m, 1H), 7.00-6.94 (m, 3H), 6.29 (d, J = 9.5 Hz, 1H), 5.68 (d, J = 6.2 Hz, |
| | 1H), 5.53 (dd, J = 6.6 Hz, 1H), 5.08-5.00 (m, 1H), 3.53 (d, J = 6.9 Hz, 1H), 3.22-3.08 (m, |
| | 1H), 2.94-2.85 (m, 2H), 2.78-2.66 (m, 3H), 2.52-2.41 (m, 1H), 2.12-1.90 (m, 2H), 1.19- |
| | 1.14 (m, 3H). |
| 518 | LCMS (ESI) [M + H]⁺: 520.3 |
| | ¹H NMR (400 MHz, DMSO-d₆) δ 11.56 (bs, 1H), 10.20 (s, 1H), 7.42-7.37 (m, 1H), 7.27 (s, |
| | 1H), 7.25-7.19 (m, 1H), 7.12-7.08 (m, 2H), 7.00 (s, 1H), 6.29 (d, J = 9.5 Hz, 1H), 5.81 (d, |
| | J = 5.7 Hz, 1H), 5.31 (bd, J = 8.3, 4.1 Hz, 1H), 5.03-4.96 (m, 1H), 3.59-3.46 (m, 1H), 3.31- |
| | 3.24 (m, 1H), 3.13 (dd, J = 27.4, 11.1 Hz, 1H), 2.94-2.85 (m, 2H), 2.77-2.70 (m, 1H), |
| | 2.50-2.42 (m, 1H), 2.21-2.14 (m, 1H), 2.14-1.90 (m, 2H), 1.15 (d, J = 6.8 Hz, 3H). |

### Example 65: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide

### Step 1: tert-butyl 4-oxo-3-(pyridin-4-yl)piperidine-1-carboxylate

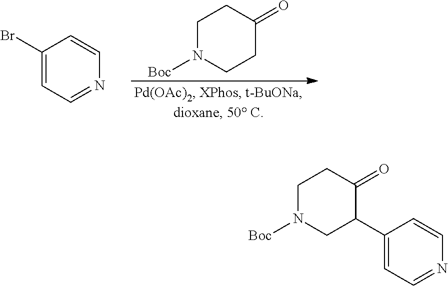 [SMILES: Brc1ccncc1 ; CC(C)(C)OC(=O)N1CCC(=O)CC1 ; CC(C)(C)OC(=O)N1CCC(=O)C(c2ccncc2)C1]

To a mixture of 4-bromopyridine (30.00 g, 189.9 mmol) in dioxane (300 mL) were added tert-butyl 4-oxopiperidine-1-carboxylate (490.43 g, 246.9 mmol), t-BuONa (54.70 g, 569.6 mmol), Pd(OAc)₂ (4.30 g, 19.0 mmol) and XPhos (18.10 g, 38.0 mmol). The resulting mixture was stirred at 50° C. overnight under nitrogen atmosphere. The mixture was allowed to cool down to room temperature. The reaction was diluted with saturated aqueous NH₄Cl solution (320 mL) at 0° C. The aqueous layer was extracted with CH₂Cl₂ (3×350 mL). The combined organic layers were dried over anhydrous Na₂SO₄, filtered and concentrated under reduced pressure. The crude residue was purified by silica gel column chromatography, eluted with PE/EA (1:1) to afford tert-butyl 4-oxo-3-(pyridin-4-yl)piperidine-1-carboxylate (31.50 g, 60% yield) as a yellow solid.

LCMS (ESI) [M+H]⁺: 277.

### Step 2: tert-butyl 3-methyl-4-oxo-3-(pyridin-4-yl)piperidine-1-carboxylate

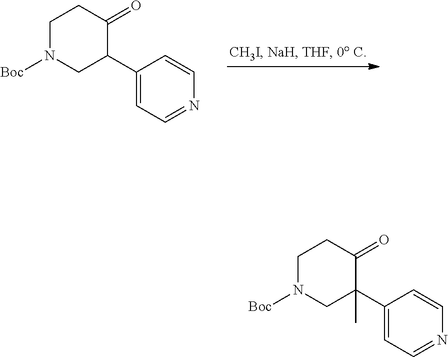 [SMILES: CC(C)(C)OC(=O)N1CCC(=O)C(c2ccncc2)C1 ; CC(C)(C)OC(=O)N1CCC(=O)C(C)(c2ccncc2)C1]

To a mixture of tert-butyl 4-oxo-3-(pyridin-4-yl)piperidine-1-carboxylate (5.00 g, 18.1 mmol) and iodomethane (2.83 g, 23.6 mmol) in THF (50 mL) was added NaH (60% in mineral oil, 0.90 g, 36.2 mmol) in portions at 0° C. The resulting mixture was stirred at room temperature for 2 h under nitrogen atmosphere. The reaction was quenched by the addition of water (60 mL) at 0° C. The resulting mixture was extracted with EtOAc (3×70 mL). The combined organic layers were dried over anhydrous Na₂SO₄, filtered and concentrated under reduced pressure. The crude residue was purified by silica gel column chromatography, eluted with PE/EA (1:1) to afford tert-butyl 3-methyl-4-oxo-3-(pyridin-4-yl)piperidine-1-carboxylate (3.80 g, 72% yield) as a light yellow solid.

LCMS (ESI) [M+H]⁺: 291.

### Step 3: tert-butyl 4,4-difluoro-3-methyl-3-(pyridin-4-yl)piperidine-1-carboxylate

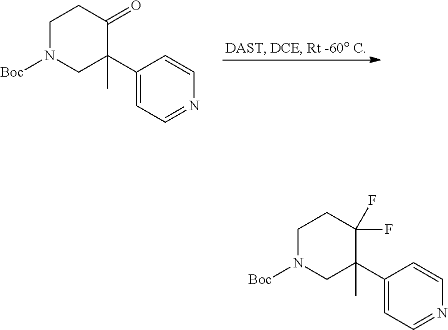 [SMILES: CC(C)(C)OC(=O)N1CCC(=O)C(C)(c2ccncc2)C1 ; CC(C)(C)OC(=O)N1CCC(F)(F)C(C)(c2ccncc2)C1]

To a mixture of tert-butyl 3-methyl-4-oxo-3-(pyridin-4-yl)piperidine-1-carboxylate (2.00 g, 6.9 mmol) in DCE (20 mL) was added DAST (5.50 g, 34.4 mmol) dropwise at 0° C. under nitrogen atmosphere. The resulting mixture was stirred at 60° C. for 5 h under nitrogen atmosphere. The mixture was allowed to cool down to room temperature. The reaction was quenched with saturated aqueous sodium bicarbonate solution (25 mL) at 0° C. The resulting mixture was extracted with EtOAc (3×30 mL). The combined organic layers were dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 0% to 100% gradient in 20 min; detector, UV 254 nm) to give tert-butyl 4,4-difluoro-3-methyl-3-(pyridin-4-yl)piperidine-1-carboxylate (312 mg, 15% yield) as a brick red semi-solid.

LCMS (ESI) [M+H]⁺: 313.

### Step 4: 4-(1-(tert-butoxycarbonyl)-4,4-difluoro-3-methylpiperidin-3-yl)pyridine 1-oxide

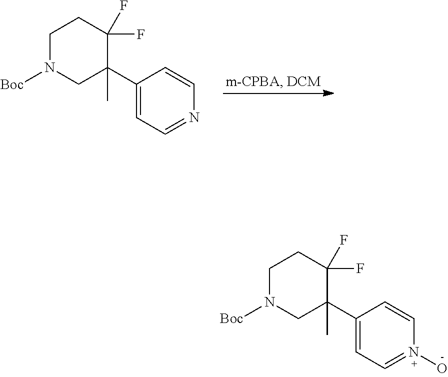

A solution of tert-butyl 4,4-difluoro-3-methyl-3-(pyridin-4-yl)piperidine-1-carboxylate (250 mg, 0.8 mmol) in DCM (8 mL) was added m-CPBA (207 mg, 1.2 mmol, 85% purity) in portions over 5 min at 0° C. The resulting mixture was stirred at 50° C. overnight under nitrogen atmosphere. The mixture was allowed to cool down to room temperature. The reaction was quenched with saturated aqueous Na₂S₂O₃ solution (10 mL) at 0° C. The aqueous layer was extracted with EtOAc (3×20 mL). The combined organic layers were dried over anhydrous Na₂SO₄, filtered and concentrated under reduced pressure. The crude residue was purified by reversed-phase flash chromatography (conditions: column, C18 silica gel; mobile phase, ACN in water (10 mmol/L NH₄HCO₃), 0% to 100% gradient in 20 min; detector, UV 254 nm) to give 4-(1-(tert-butoxycarbonyl)-4,4-difluoro-3-methylpiperidin-3-yl)pyridine 1-oxide (150 mg, 57% yield) as a yellow solid.

LCMS (ESI) [M+H]⁺: 329.

### Step 5: 4-(4,4-difluoro-3-methylpiperidin-3-yl)pyridine 1-oxide

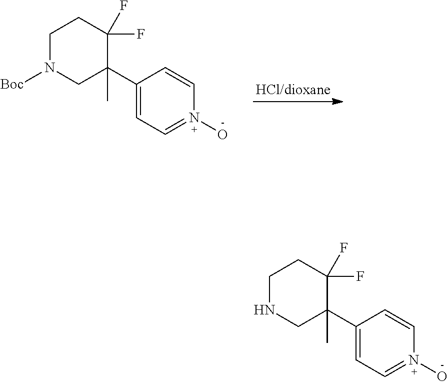

A solution of 4-(1-(tert-butoxycarbonyl)-4,4-difluoro-3-methylpiperidin-3-yl)pyridine 1-oxide (150 mg, 457 μmol) in 4 M HCl in 1,4-dioxane (6 mL) was stirred at room temperature for 2 h under an ambient atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to give 4-(4,4-difluoro-3-methylpiperidin-3-yl)pyridine 1-oxide (80 mg, 76% yield) as a white solid.

LCMS (ESI) [M+H]⁺: 229.

### Step 6: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide

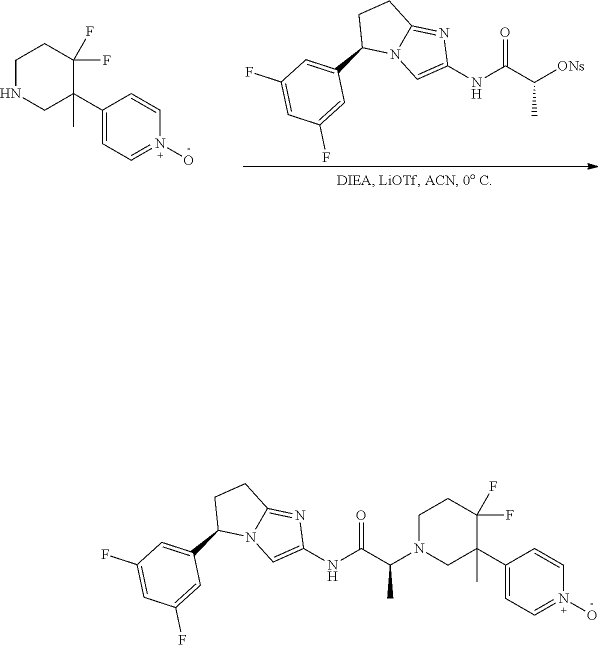

A solution of 4-(4,4-difluoro-3-methylpiperidin-3-yl)pyridine 1-oxide (70 mg, 0.3 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (181 mg, 0.4 mmol), DIEA (99 mg, 0.8 mmol) and lithium trifluoromethanesulfonate (124 mg, 0.8 mmol) in ACN (5 mL) was stirred at 0° C. for 4 days under nitrogen atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to give 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide (25 mg, 16% yield) as a light yellow oil.

LCMS (ESI) [M+H]⁺: 518.

### Step 7: 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide and 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide

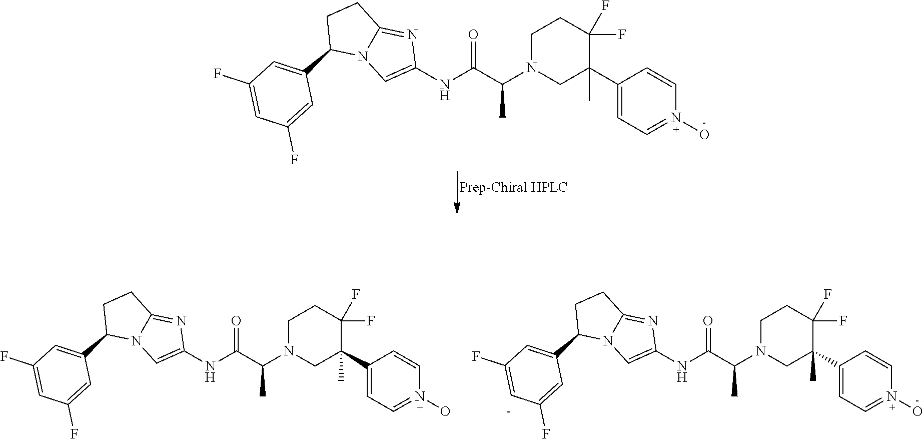

4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide (25 mg) was separated by Prep-Chiral HPLC with the following conditions (Column: NB-CHIRALCEL OD-H, 3*25 cm, 5 μm; Mobile Phase A: Hex (10 mM NH₃-MeOH), Mobile Phase B: EtOH; Flow rate: 40 mL/min; Gradient: isocratic 30% B; Wave Length: 212/268 nm; RT1 (min): 13.938; RT2 (min): 19.758) to afford first peak 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide (Method AG, Peak 1, 2.38 min; 4.8 mg, 100% d.e.) (Compound 519) as a white solid and second peak 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoro-3-methylpiperidin-3-yl)pyridine 1-oxide (Method AG, Peak 2, 3.35 min; 4.0 mg, 100% d.e.) (Compound 520) as an off white solid.

TABLE 39

| Compound# | Characterization Data |
|---|---|
| 519 | LCMS (ESI) [M + H]⁺: 518.2 |
| | ¹H NMR (400 MHz, DMSO-d₆) δ 10.08 (s, 1H), 8.21-8.08 (m, 2H), 7.57 (d, J = 6.6 Hz, 2H), |
| | 7.24-7.19 (m, 1H), 7.03-6.85 (m, 3H), 5.47-5.33 (m, 1H), 3.51 (d, J = 6.9 Hz, 1H), 3.08- |
| | 2.89 (m, 2H), 2.82-2.68 (m, 4H), 2.42-2.30 (m, 1H), 2.21-1.81 (m, 3H), 1.42 (s, 3H), |
| | 1.23 (d, J = 7.7 Hz, 3H). |
| 520 | LCMS (ESI) [M + H]⁺: 518.3 |
| | ¹H NMR (400 MHz, DMSO-d₆) δ 10.13 (s, 1H), 8.25-8.03 (m, 2H), 7.48 (d, J = 6.7 Hz, 2H), |
| | 7.22 (dd, J = 9.3, 2.3 Hz, 1H), 7.00-6.91 (m, 3H), 5.52-5.22 (m, 1H), 3.64-3.49 (m, 1H), |
| | 3.07 (d, J = 11.9 Hz, 1H), 3.00-2.64 (m, 5H), 2.60-2.33 (m, 2H), 2.22-2.05 (m, 1H), |
| | 1.95-1.85 (m, 1H), 1.43 (s, 3H), 1.19 (d, J = 6.9 Hz, 3H). |

### Example 66: 4-(6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide

### Step 1: tert-butyl 4-hydroxy-6-azaspiro[2.5]octane-6-carboxylate

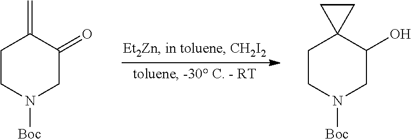 [SMILES: C=C1CCN(C(=O)OC(C)(C)C)CC1=O ; CC(C)(C)OC(=O)N1CCC2(CC2)C(O)C1]

To a mixture of tert-butyl 3-hydroxy-4-methylenepiperidine-1-carboxylate (5.00 g, 23.4 mmol) in toluene (80 mL) was added diethylzinc in toluene (3.07 g, 46.9 mmol, 2 M) dropwise over 8 min at −30° C. under nitrogen atmosphere. The resulting mixture was stirred at −30° C. for 30 min. To the above mixture was added diiodomethane (18.84 g, 70.3 mmol) dropwise over 10 min at −30° C. The resulting mixture was stirred at room temperature for an additional 3 h. The reaction was quenched with saturated aqueous NH₄Cl solution (85 mL) at 0° C. The aqueous layer was extracted with EtOAc (3×90 mL). The combined organic layers were dried over anhydrous Na₂SO₄, filtered and concentrated under reduced pressure. The crude residue was purified by silica gel column chromatography, eluted with PE/EA (1:1.5) to afford tert-butyl 4-hydroxy-6-azaspiro[2.5]octane-6-carboxylate (3.60 g, 68% yield) as a white solid.

LCMS (ESI) [M+H]⁺: 228.

### Step 2: tert-butyl 4-oxo-6-azaspiro[2.5]octane-6-carboxylate

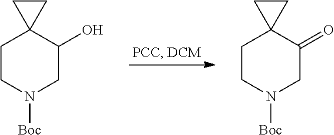 [SMILES: CC(C)(C)OC(=O)N1CCC2(CC2)C(O)C1 ; CC(C)(C)OC(=O)N1CCC2(CC2)C(=O)C1]

To a mixture of tert-butyl 4-hydroxy-6-azaspiro[2.5]octane-6-carboxylate (3.6 g, 15.8 mmol) in DCM (40 mL) was added PCC (10.24 g, 47.5 mmol) at 0° C. The resulting mixture was stirred at room temperature for 3 h under nitrogen atmosphere. The resulting mixture was filtered through a plug of celite. The filter cake was washed with CH₂Cl₂ (3×50 mL). The filtrate was concentrated under reduced pressure to afford tert-butyl 4-oxo-6-azaspiro[2.5]octane-6-carboxylate (2.50 g, 70% yield) as a yellow solid, which was used directly in the next reaction without further purification

LCMS (ESI) [M+H]⁺: 226.

### Step 3: tert-butyl 4-(((trifluoromethyl)sulfonyl)oxy)-6-azaspiro[2.5]oct-4-ene-6-carboxylate

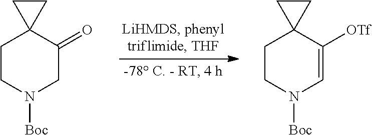 [SMILES: CC(C)(C)OC(=O)N1CCC2(CC2)C(=O)C1 ; CC(C)(C)OC(=O)N1C=C(OS(=O)(=O)C(F)(F)F)C2(CC1)CC2]

To a mixture of tert-butyl 4-oxo-6-azaspiro[2.5]octane-6-carboxylate (2.50 g, 11.1 mmol) in THF (45 mL) was added LiHMDS (2.04 g, 12.2 mmol, 1 M in THF) dropwise over 8 min at −78° C. under nitrogen atmosphere. The resulting mixture was stirred at −78° C. for 2 h. Phenyl triflimide (4.16 g, 11.7 mmol) was added in portions over 10 min at −78° C. The resulting mixture was stirred at room temperature for additional 4 h. The reaction was quenched with saturated aqueous NH₄Cl solution (50 mL) at 0° C. The aqueous layer was extracted with EtOAc (3×55 mL). The combined organic layers were dried over anhydrous Na₂SO₄, filtered and concentrated under reduced pressure. The crude residue was purified by silica gel column chromatography, eluted with PE/EA (3:1) to afford tert-butyl 4-(((trifluoromethyl)sulfonyl)oxy)-6-azaspiro[2.5]oct-4-ene-6-carboxylate (2.10 g, 53% yield) as a brown solid.

LCMS (ESI) [M+H]⁺: 358.

### Step 4: tert-butyl 4-(pyridin-4-yl)-6-azaspiro[2.5]oct-4-ene-6-carboxylate

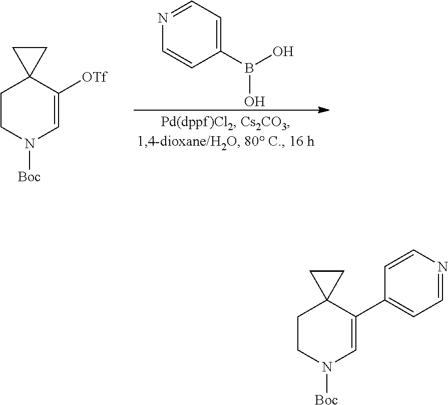 [SMILES: CC(C)(C)OC(=O)N1C=C(OS(=O)(=O)C(F)(F)F)C2(CC1)CC2 ; OB(O)c1ccncc1 ; CC(C)(C)OC(=O)N1C=C(c2ccncc2)C2(CC1)CC2]

To a mixture of tert-butyl 4-(((trifluoromethyl)sulfonyl)oxy)-6-azaspiro[2.5]oct-4-ene-6-carboxylate (2.10 g, 5.9 mmol) in dioxane (24 mL) were added H₂O (6 mL), Cs₂CO₃ (5.74 g, 17.6 mmol), pyridin-4-ylboronic acid (1.44 g, 11.8 mmol) and Pd(dppf)Cl₂ (0.86 g, 1.2 mmol). The resulting mixture was stirred at 80° C. for 16 h. The mixture was allowed to cool down to room temperature. The reaction mixture was diluted with water (35 mL) at 0° C. The aqueous layer was extracted with EtOAc (3×40 mL). The combined organic layers were dried over anhydrous Na₂SO₄, filtered and concentrated under reduced pressure. The crude residue was purified by silica gel column chromatography, eluted with PE/EA (1:1) to afford tert-butyl 4-(pyridin-4-yl)-6-azaspiro[2.5]oct-4-ene-6-carboxylate (350 mg, 21% yield) as a brown oil.

LCMS (ESI) [M+H]⁺: 287.

### Step 5: tert-butyl 4-(pyridin-4-yl)-6-azaspiro[2.5]octane-6-carboxylate

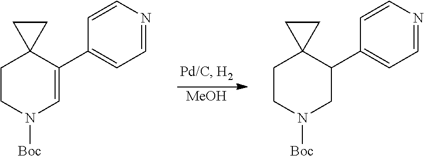 [SMILES: "MeOH" ; CC(C)(C)OC(=O)N1C=C(c2ccncc2)C2(CC1)CC2 ; CC(C)(C)OC(=O)N1CCC2(CC2)C(c2ccncc2)C1]

A solution of tert-butyl 4-(pyridin-4-yl)-6-azaspiro[2.5]oct-4-ene-6-carboxylate (340 mg, 1.2 mmol) and Pd/C (126 mg, 1.2 mmol) in MeOH (10 mL) was stirred at room temperature for 1 h under hydrogen atmosphere. The resulting mixture was filtered through a plug of celite. The filter cake was washed with MeOH (3×20 mL). The filtrate was concentrated under reduced pressure. The crude residue was purified by reversed-phase flash chromatography (conditions: column, C18 silica gel; mobile phase, MeCN in water (10 mmol/L NH₄HCO₃), 0% to 100% gradient in 30 min; detector, UV 254 nm) to give tert-butyl 4-(pyridin-4-yl)-6-azaspiro[2.5]octane-6-carboxylate (170 mg, 50% yield) as a yellow oil.

LCMS (ESI) [M+H]⁺: 289.

### Step 6: 4-(6-(tert-butoxycarbonyl)-6-azaspiro[2.5]octan-4-yl)pyridine 1-oxide

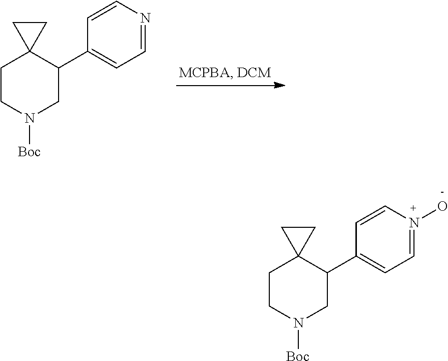

To a mixture of tert-butyl 4-(pyridin-4-yl)-6-azaspiro[2.5]octane-6-carboxylate (160 mg, 0.6 mmol) in DCM (6 mL) were added MCPBA (239 mg, 1.4 mmol, 85% purity) in portions at 0° C. The resulting mixture was stirred at room temperature for 1 h under nitrogen atmosphere. The reaction mixture was quenched with sodium thiosulfate solution (10 mL) at 0° C. The aqueous layer was extracted with EtOAc (3×15 mL). The combined organic layers were dried over anhydrous Na₂SO₄, filtered and concentrated under reduced pressure. The crude residue was purified by reversed-phase flash chromatography (conditions: column, C18 silica gel; mobile phase, MeCN in water (10 mmol/L NH₄HCO₃), 0% to 100% gradient in 30 min; detector, UV 254 nm) to give 4-(6-(tert-butoxycarbonyl)-6-azaspiro[2.5]octan-4-yl)pyridine 1-oxide (110 mg, 65% yield) as a yellow oil.

LCMS (ESI) [M+H]⁺: 305.

### Step 7: 4-(6-azaspiro[2.5]octan-4-yl)pyridine 1-oxide

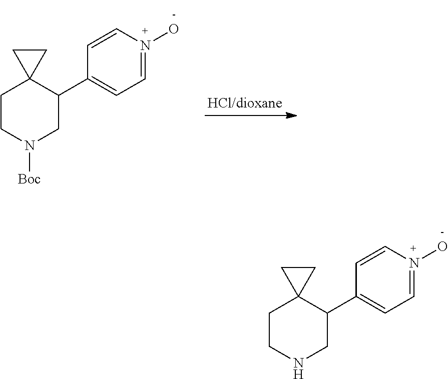

A solution of 4-(6-(tert-butoxycarbonyl)-6-azaspiro[2.5]octan-4-yl)pyridine 1-oxide (100 mg, 0.33 mmol) in 4 M HCl in 1,4-dioxane (6 mL) was stirred for 2 h at room temperature under an ambient atmosphere. The resulting mixture was concentrated under vacuum to give 4-(6-azaspiro[2.5]octan-4-yl)pyridine 1-oxide (70 mg, crude) as a yellow solid, which was used in the next step without further purification.

LCMS (ESI) [M+H]⁺: 205.

### Step 8: 4-(6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide

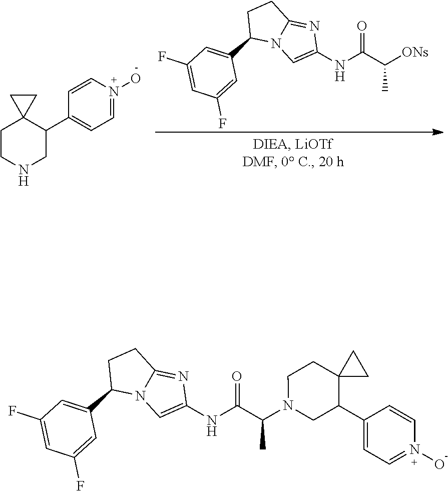

A solution of 4-(6-azaspiro[2.5]octan-4-yl)pyridine 1-oxide (70 mg, 0.4 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (208 mg, 0.4 mmol), DIEA (136 mg, 1.1 mmol) and lithium trifluoromethanesulfonate (164 mg, 1.1 mmol) in DMF (6 mL) was stirred at 0° C. for 3 days under nitrogen atmosphere. The resulting mixture was concentrated under vacuum. The crude residue was directly purified by reversed-phase flash chromatography (conditions: column, C18 silica gel; mobile phase, ACN in water (0.1% formic acid), 0% to 100% gradient in 20 min; detector, UV 254 nm and UV 220 nm) to give 4-(6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide (50 mg, 29.5% yield) as a yellow oil.

LCMS (ESI) [M+H]⁺: 494.

### Step 9: 4-((S)-6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide and 4-((R)-6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide

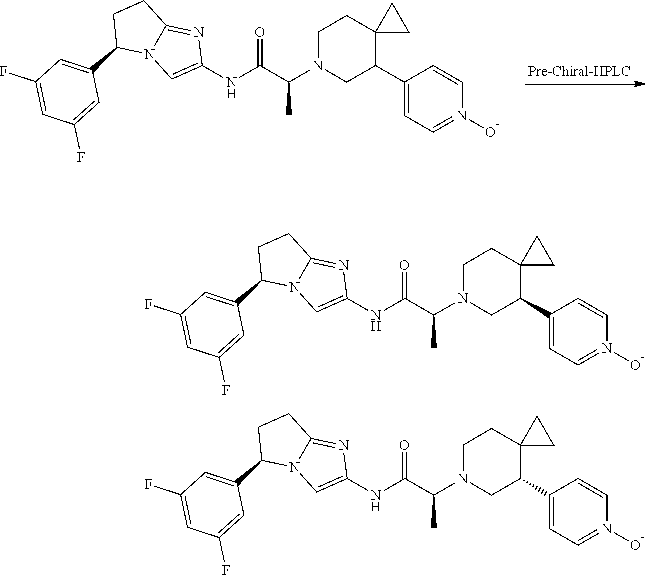

4-(6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide (49 mg) was separated by Prep-Chiral HPLC with the following conditions (Column: CHIRAL ART Cellulose-SZ, 3*25 cm, 5 μm; Mobile Phase A: MtBE (10 mM NH₃-MeOH), Mobile Phase B: MeOH; Flow rate: 40 mL/min; Gradient: isocratic 50% B; Wave Length: 212/266 nm; RT1 (min): 6.57; RT2 (min): 10.64) to afford first peak 4-((S)-6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide (Compound 521) (Method AH, Peak 1, 1.16 min; 19.7 mg, 100% d.e.) as a white solid (RT1) and second peak 4-((R)-6-((S)-1-(((R)-5-(3,5-difluorophenyl)-6,7-dihydro-5H-pyrrolo[1,2-a]imidazol-2-yl)amino)-1-oxopropan-2-yl)-6-azaspiro[2.5]octan-4-yl)pyridine 1-oxide (Compound 522) (Method AH, Peak 2, 1.76 min; 17.4 mg, 100% d.e.) as a white solid (RT2).

TABLE 40

| Compound# | Characterization Data |
|---|---|
| 521 | LCMS (ESI) [M + H]⁺: 494.10 |
| | ¹H NMR (400 MHz, DMSO-d₆) δ 9.91 (s, 1H), 8.08 (d, J = 7.2 Hz, 2H), 7.49 (d, J = 6.4 Hz, |
| | 2H), 7.24-7.19 (m, 1H), 6.96-6.94 (m, 3H), 5.39-5.35 (m, 1H), 3.40-3.38 (m, 1H), |
| | 3.00-2.89 (m, 3H), 2.80-2.72 (m, 3H), 2.49-2.31 (m, 3H), 1.78-1.66 (m, 1H), 1.15 (d, J = 6.8 |
| | Hz, 3H), 1.11-1.02 (m, 1H), 0.49-0.22 (m, 4H). |
| 522 | LCMS (ESI) [M + H]⁺: 494.10 |
| | ¹H NMR (400 MHz, DMSO-d₆) δ 9.88 (s, 1H), 8.03 (d, J = 6.9 Hz, 2H), 7.44 (d, J = 6.6 Hz, |
| | 2H), 7.24-7.19 (m, 1H), 6.95-6.93 (m, 3H), 5.39-5.36 (m, 1H), 3.44-3.40 (m, 2H), |
| | 3.32-3.30 (m, 1H), 2.99-2.93 (m, 2H), 2.81-2.51 (m, 4H), 2.39-2.31 (m, 2H), 1.78-1.50 (m, |
| | 1H), 1.14 (d, J = 6.9 Hz, 3H), 0.35-0.20 (m, 4H). |

### Example 66: (2S)-2-(4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

### Step 1: benzyl 4,4-difluoro-3-(5-(hydroxymethyl)-6-methoxypyridin-3-yl)piperidine-1-carboxylate

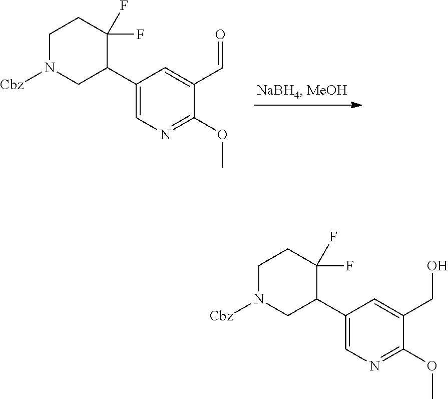 [SMILES: COc1ncc(C2CN(C(=O)OCc3ccccc3)CCC2(F)F)cc1C=O ; COc1ncc(C2CN(C(=O)OCc3ccccc3)CCC2(F)F)cc1CO]

A solution of benzyl 4,4-difluoro-3-(5-formyl-6-methoxypyridin-3-yl)piperidine-1-carboxylate (500 mg, 1.3 mmol) in MeOH (10 mL) was stirred at 0° C. for 10 min under nitrogen atmosphere. To the above mixture was added NaBH₄ (73 mg, 1.9 mmol) in portions over 5 min at 0° C. The resulting mixture was stirred at room temperature for additional 2 h. The reaction was quenched with water solution (12 mL) at 0° C. The aqueous layer was extracted with EtOAc (3×15 mL). The combined organic layers were dried over anhydrous Na₂SO₄, filtered and concentrated under reduced pressure. The crude residue was purified by silica gel column chromatography, eluted with PE/EA (1:1) to afford benzyl 4,4-difluoro-3-(5-(hydroxymethyl)-6-methoxypyridin-3-yl)piperidine-1-carboxylate (350 mg, 70% yield) as a colorless oil.

LCMS (ESI) [M+H]⁺: 393.

### Step 2: (5-(4,4-difluoropiperidin-3-yl)-2-methoxypyridin-3-yl)methanol

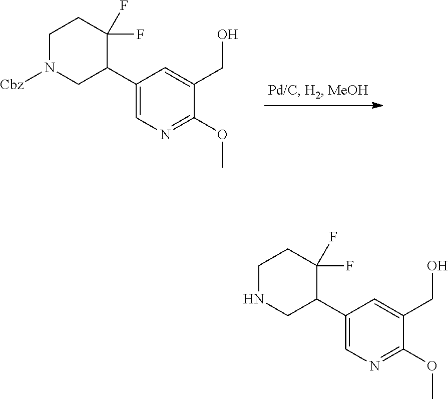 [SMILES: COc1ncc(C2CN(C(=O)OCc3ccccc3)CCC2(F)F)cc1CO ; COc1ncc(C2CNCCC2(F)F)cc1CO]

To a mixture of benzyl 4,4-difluoro-3-(5-(hydroxymethyl)-6-methoxypyridin-3-yl)piperidine-1-carboxylate (350 mg, 0.9 mmol) in MeOH (5 mL) was added Pd/C (95 mg, 90.0 μmol) at room temperature under hydrogen atmosphere. The resulting mixture was stirred at room temperature for 2 h under hydrogen atmosphere. The resulting mixture was filtered through celite. The filter cake was washed with MeOH (3×10 mL). The filtrate was concentrated under reduced pressure. The crude residue was purified by reversed-phase flash chromatography (conditions: column, C18 silica gel; mobile phase, ACN in water (0.1% formic acid), 0% to 100% gradient in 20 min; detector, UV 254 nm) to afford (5-(4,4-difluoropiperidin-3-yl)-2-methoxypyridin-3-yl)methanol (200 mg, 87% yield) as a colorless oil.

LCMS (ESI) [M+H]⁺: 259.

### Step 3: (2S)-2-(4,4-difluoro-3-(5-(hydroxymethyl)-6-methoxypyridin-3-yl)piperidin-1-yl)-N—((R)-5-(3,5-difluorophenyl)-6,7-dihydro-5H-pyrrolo[1,2-a]imidazol-2-yl)propanamide

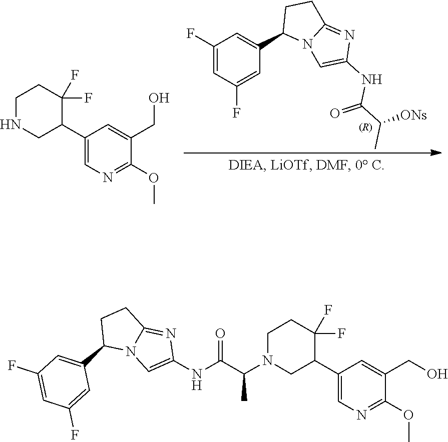

A solution of (5-(4,4-difluoropiperidin-3-yl)-2-methoxypyridin-3-yl)methanol (190 mg, 0.7 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (435 mg, 0.9 mmol), DIEA (238 mg, 1.8 mmol) and lithium trifluoromethanesulfonate (298 mg, 1.9 mmol) in DMF (8 mL) was stirred at 0° C. for 4 days under nitrogen atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to give (2S)-2-(4,4-difluoro-3-(5-(hydroxymethyl)-6-methoxypyridin-3-yl)piperidin-1-yl)-N—((R)-5-(3,5-difluorophenyl)-6,7-dihydro-5H-pyrrolo[1,2-a]imidazol-2-yl)propanamide (120 mg, 30% yield) as a yellow solid.

LCMS (ESI) [M+H]⁺: 548.

### Step 4: (2S)-2-(4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

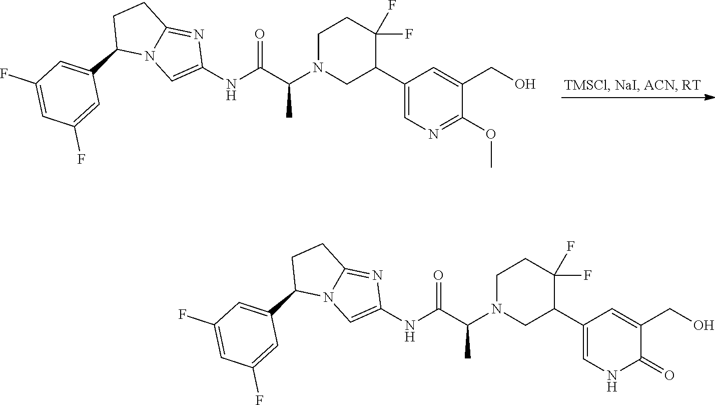 [SMILES: COc1ncc(C2CN([C@@H](C)C(=O)Nc3cn4c(n3)CC[C@@H]4c3cc(F)cc(F)c3)CCC2(F)F)cc1CO ; C[C@@H](C(=O)Nc1cn2c(n1)CC[C@@H]2c1cc(F)cc(F)c1)N1CCC(F)(F)C(c2c[nH]c(=O)c(CO)c2)C1]

To a mixture of (2S)-2-(4,4-difluoro-3-(5-(hydroxymethyl)-6-methoxypyridin-3-yl)piperidin-1-yl)-N—((R)-5-(3,5-difluorophenyl)-6,7-dihydro-5H-pyrrolo[1,2-a]imidazol-2-yl)propanamide (110 mg, 0.2 mmol) in ACN (5 mL) were added NaI (151 mg, 1.0 mmol) and TMSCl (109 mg, 1.0 mmol) dropwise over 2 min at 0° C. under nitrogen atmosphere. The reaction was stirred at room temperature for 3 h. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to give (2S)-2-(4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (50 mg, 47% yield) as a light yellow solid.

LCMS (ESI) [M+H]⁺: 534.

### Step 5: (S)-2-((S)-4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide and (S)-2-((R)-4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

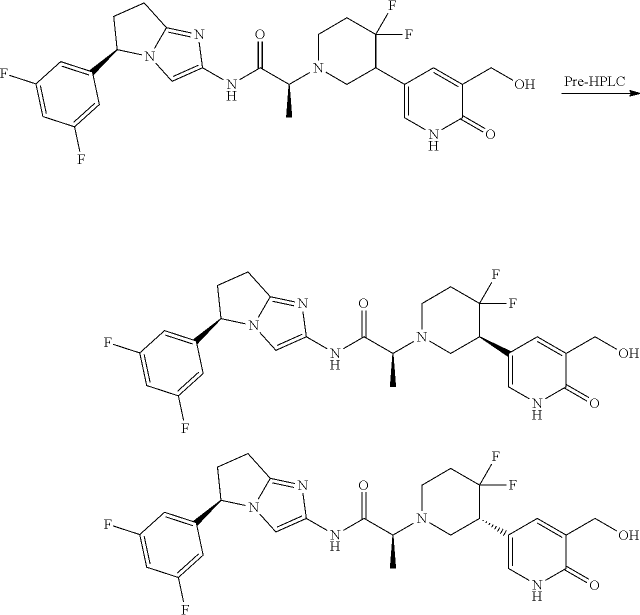 [SMILES: C[C@@H](C(=O)Nc1cn2c(n1)CC[C@@H]2c1cc(F)cc(F)c1)N1CCC(F)(F)C(c2c[nH]c(=O)c(CO)c2)C1 ; C[C@@H](C(=O)Nc1cn2c(n1)CC[C@@H]2c1cc(F)cc(F)c1)N1CCC(F)(F)[C@@H](c2c[nH]c(=O)c(CO)c2)C1 ; C[C@@H](C(=O)Nc1cn2c(n1)CC[C@@H]2c1cc(F)cc(F)c1)N1CCC(F)(F)[C@H](c2c[nH]c(=O)c(CO)c2)C1]

(2S)-2-(4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (50 mg) was separated by Prep-HPLC (Column: Xselect CSH Prep C18, 30*150 mm 5 μm; Mobile Phase A: Water (0.1% FA), Mobile Phase B: MeOH; Flow rate: 60 mL/min; Gradient: 27% to 46% B in 15 min; Wave Length: 254 nm/220 nm) RT1 (min): 11.6, RT2 (min): 14.76) to afford first peak (S)-2-((S)-4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 523) (Method AI, Peak 1, 2.29 min; 5.9 mg, 99% d.e.) as a white solid and second peak (S)-2-((R)-4,4-difluoro-3-(5-(hydroxymethyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 524) (5.3 mg, 96% d.e.) as a white solid.

TABLE 41

| Compound# | Characterization Data |
|---|---|
| 523 | LCMS (ESI) [M + H]⁺: 534.2 |
| | ¹H NMR (400 MHz, DMSO-d₆) δ 11.52 (bs, 1H), 10.09 (s, 1H), 7.41 (s, 1H), 7.25-7.15 (m, |
| | 2H), 6.99-6.95 (m, 3H), 5.39-5.35 (m, 1H), 5.09-5.02 (m, 1H), 4.26 (d, J = 5.1 Hz, 2H), |
| | 3.52-3.42 (m, 1H), 3.25-3.13 (m, 1H), 3.04-2.78 (m, 5H), 2.68-2.57 (m, 2H), |
| | 2.42-2.30 (m, 1H), 2.10-1.89 (m, 2H), 1.17 (d, J = 6.8 Hz, 3H). |
| 524 | LCMS (ESI) [M + H]⁺: 534.2 |
| | ¹H NMR (400 MHz, DMSO-d₆) δ 11.53 (bs, 1H), 10.09 (s, 1H), 7.42 (s, 1H), 7.24-7.18 (m, |
| | 2H), 6.97-6.95 (m, 3H), 5.39-5.36 (m, 1H), 5.07 (bs, 1H), 4.27 (s, 2H), 3.52-3.50 (m, |
| | 1H), 3.23-3.10 (m, 1H), 3.02-2.82 (m, 4H), 2.80-2.71 (m, 2H), 2.45-2.35 (m, 2H), |
| | 2.12-2.09 (m, 2H), 1.16 (d, J = 6.8 Hz, 3H). |

### Example 67: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide

### Step 1: tert-butyl 4,4-difluoro-3-(2-formylpyridin-4-yl)piperidine-1-carboxylate

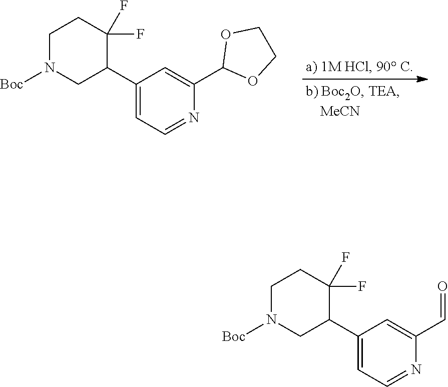 [SMILES: CC(C)(C)OC(=O)N1CCC(F)(F)C(c2ccnc(C3OCCO3)c2)C1 ; CC(C)(C)OC(=O)N1CCC(F)(F)C(c2ccnc(C=O)c2)C1]

A solution of tert-butyl 3-(2-(1,3-dioxolan-2-yl)pyridin-4-yl)-4,4-difluoropiperidine-1-carboxylate (2.00 g, 5.4 mmol) in 1 M HCl in water (25 mL) was stirred at 90° C. for 3 h under nitrogen atmosphere. The mixture was allowed to cool down to room temperature. The resulting mixture was concentrated under vacuum. The residue was dissolved in MeCN (35 mL). TEA (2.73 g, 27.0 mmol) and Boc₂O (3.54 g, 16.2 mmol) were added to the solution at 0° C. The resulting mixture was stirred at room temperature for additional 2 h. The resulting mixture was concentrated under reduced pressure. The crude residue was directly purified by silica gel column chromatography, eluted with PE/EA (3:1) to afford tert-butyl 4,4-difluoro-3-(2-formylpyridin-4-yl)piperidine-1-carboxylate (1.00 g, 57% yield) as a yellow oil.

LCMS (ESI) [M+H]⁺: 327.

### Step 2: tert-butyl 4,4-difluoro-3-(2-(hydroxymethyl)pyridin-4-yl)piperidine-1-carboxylate

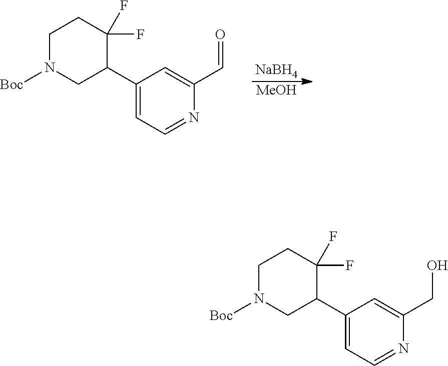

To a mixture of tert-butyl 4,4-difluoro-3-(2-formylpyridin-4-yl)piperidine-1-carboxylate (1.00 g, 3.1 mmol) in MeOH (15 mL) were added NaBH₄ (348 mg, 9.2 mmol) in portions at 0° C. under nitrogen atmosphere. The resulting mixture was stirred at room temperature for 2 h under nitrogen atmosphere. The reaction was quenched with water at 0° C. The resulting mixture was extracted with EtOAc (3×30 mL). The combined organic layers were dried over anhydrous Na₂SO₄, filtered and concentrated under reduced pressure. The crude residue was purified by silica gel column chromatography, eluted with PE/EA (2:1) to afford tert-butyl 4,4-difluoro-3-(2-(hydroxymethyl)pyridin-4-yl)piperidine-1-carboxylate (650 mg, 65% yield) as a colorless oil.

LCMS (ESI) [M+H]⁺: 329.

### Step 3: 4-(1-(tert-butoxycarbonyl)-4,4-difluoropiperidin-3-yl)-2-(hydroxymethyl)pyridine 1-oxide

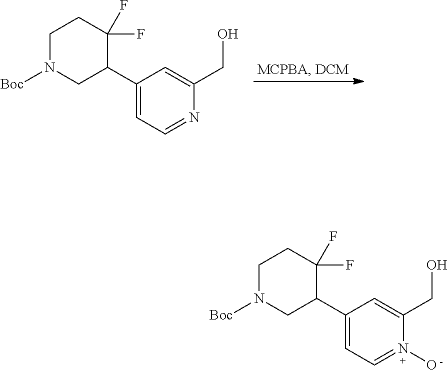

To a mixture of tert-butyl 4,4-difluoro-3-(2-(hydroxymethyl)pyridin-4-yl)piperidine-1-carboxylate (620 mg, 1.9 mmol) in DCM (8 mL) was added MCPBA (978 mg, 5.7 mmol, 85% purity) in portions at 0° C. under nitrogen atmosphere. The resulting mixture was stirred at room temperature for 1 h under nitrogen atmosphere. The reaction was quenched with saturated aqueous Na₂S₂O₃ solution (10 mL) at 0° C. The resulting mixture was extracted with EtOAc (3×20 mL). The combined organic layers were dried over anhydrous Na₂SO₄, filtered and concentrated under reduced pressure. The crude residue was purified by silica gel column chromatography, eluted with PE/EA (3:2) to afford 4-(1-(tert-butoxycarbonyl)-4,4-difluoropiperidin-3-yl)-2-(hydroxymethyl)pyridine 1-oxide (420 mg, 65% yield) as a yellow solid.

LCMS (ESI) [M+H]⁺: 345.

### Step 4: 4-(4,4-difluoropiperidin-3-yl)-2-(hydroxymethyl)pyridine 1-oxide

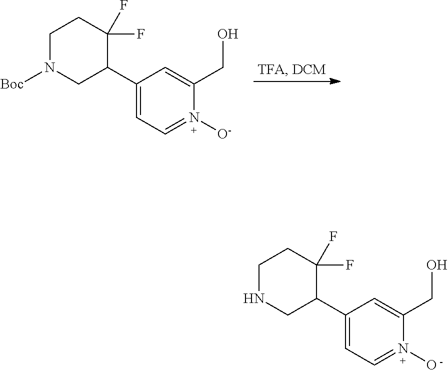

To a mixture of 4-(1-(tert-butoxycarbonyl)-4,4-difluoropiperidin-3-yl)-2-(hydroxymethyl)pyridine 1-oxide (200 mg, 0.6 mmol) in DCM (6 mL) was added TFA (3 mL) dropwise at 0° C. The resulting mixture was stirred at room temperature for 1 h under nitrogen atmosphere. The resulting mixture was concentrated under vacuum to afford 4-(4,4-difluoropiperidin-3-yl)-2-(hydroxymethyl)pyridine 1-oxide (200 mg, crude) as a yellow oil, which was used in the next step without further purification.

LCMS (ESI) [M+H]⁺: 245.

### Step 5: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide

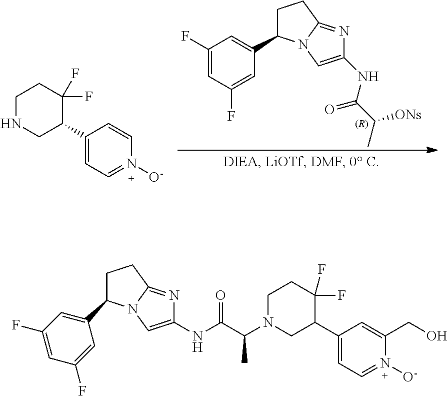

To a mixture of (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (100 mg, 0.2 mmol) in DMF (4 mL) were added 4-(4,4-difluoropiperidin-3-yl)-2-(hydroxymethyl)pyridine 1-oxide (60 mg, 0.2 mmol), lithium trifluoromethanesulfonate (158 mg, 1.0 mmol) and DIEA (131 mg, 1.0 mmol) dropwise at 0° C. The resulting mixture was stirred at 0° C. for 5 days under nitrogen atmosphere. The reaction mixture was directly purified by reversed-phase flash chromatography (conditions: column, C18 silica gel; mobile phase, ACN in water (0.1% formic acid), 0% to 100% gradient in 20 min; detector, UV 254 nm and UV 220 nm) to give 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide (40 mg, 37% yield) as a yellow solid.

LCMS (ESI) [M+H]⁺: 534.

### Step 6: 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide and 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide

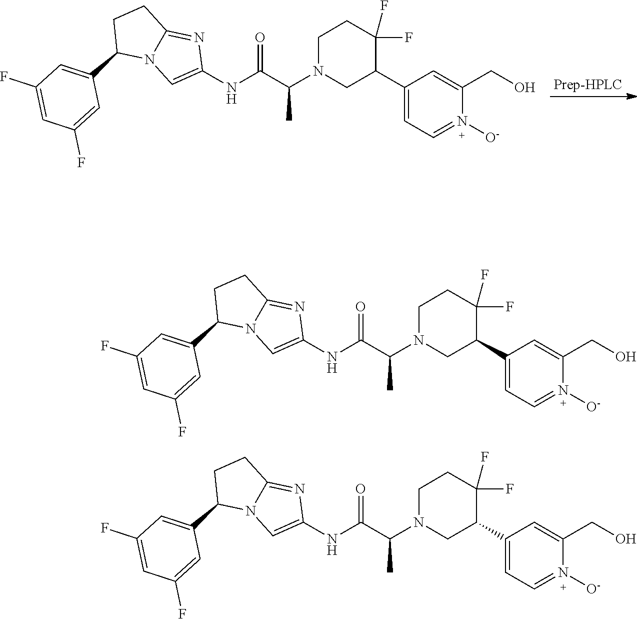

4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide (39 mg) was separated by Prep-HPLC with the following conditions (Column: Xselect CSH Prep C18, 30*150 mm 5 μm; Mobile Phase A: Water (0.1% FA), Mobile Phase B: ACN; Flow rate: 60 mL/min; Gradient: 6% to 24% B in 15 min; Wave Length: 254 nm/220 nm; RT1 (min): 16.02; RT2 (min): 17.56) to afford first peak 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide (Compound 525) (Method AJ, Peak 2, 2.72 min; 9.5 mg, 95% d.e.) as a white solid and second peak 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(hydroxymethyl)pyridine 1-oxide (Compound 526) (Method AJ, Peak 1, 1.08 min; 8.1 mg, 100% d.e.) as a white solid.

TABLE 42

| Compound# | Characterization Data |
|---|---|
| 525 | LCMS (ESI) [M + H]⁺: 534.1 |
| | ¹H NMR (300 MHz, DMSO-d₆) δ 10.13 (s, 1H), 8.18 (d, J = 6.7 Hz, 1H), 7.51 (s, 1H), 7.30 |
| | (d, J = 6.2 Hz, 1H), 7.29-7.22 (m, 1H), 6.99-6.95 (m, 3H), 5.67-5.61 (m, 1H), |
| | 5.41-5.34 (m, 1H), 4.56 (d, J = 5.3 Hz, 2H), 3.56-3.45 (m, 2H), 3.01-2.88 (m, 4H), |
| | 2.84-2.63 (m, 3H), 2.43-2.31 (m, 1H), 2.19-1.97 (m, 2H), 1.18 (d, J = 6.9 Hz, 3H). |
| 526 | LCMS (ESI) [M + H]⁺: 534.1 |
| | ¹H NMR (400 MHz, DMSO-d₆) δ 10.13 (s, 1H), 8.19 (d, J = 6.7 Hz, 1H), 7.53-7.47 (m, 1H), |
| | 7.33-7.29 (m, 1H), 7.28-7.19 (m, 1H), 7.02-6.86 (m, 3H), 5.69-5.61 (m, 1H), |
| | 5.42-5.36 (m, 1H), 4.56 (d, J = 4.7 Hz, 2H), 3.60-3.42 (m, 2H), 3.02-2.72 (m, 6H), |
| | 2.41-2.32 (m, 2H), 2.19-1.92 (m, 2H), 1.17 (d, J = 6.9 Hz, 3H). |

### Example 68: (2S)-2-(4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide

### Step 1: 5-(4,4-difluoropiperidin-3-yl)pyrazin-2(1H)-one

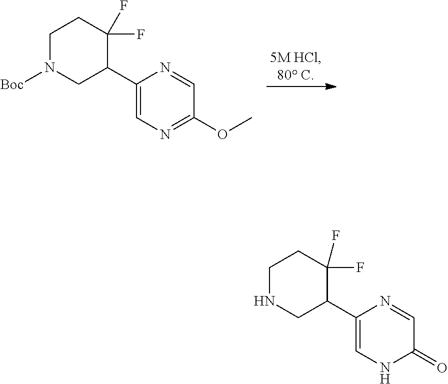 [SMILES: COc1cnc(C2CN(C(=O)OC(C)(C)C)CCC2(F)F)cn1 ; O=c1cnc(C2CNCCC2(F)F)c[nH]1]

A solution of tert-butyl 4,4-difluoro-3-(5-methoxypyrazin-2-yl)piperidine-1-carboxylate (500 mg, 1.5 mmol) in 5 M HCl in water (10 mL) was stirred at 80° C. for 48 h. The mixture was allowed to cool down to room temperature. The resulting mixture was concentrated under vacuum to afford 5-(4,4-difluoropiperidin-3-yl)pyrazin-2(1H)-one (330 mg, crude) as a white solid, which was used directly in the next step without further purification.

LCMS (ESI) [M+H]⁺. 216.

### Step 2: (2S)-2-(4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide

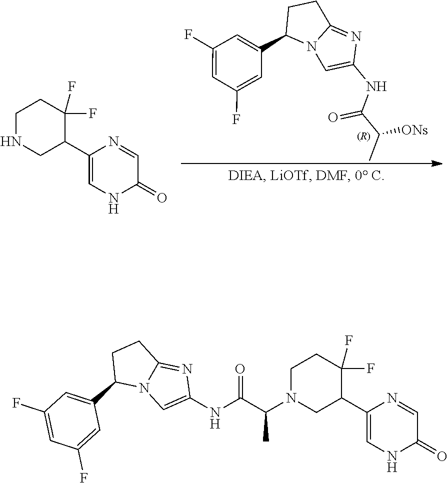

To a mixture of (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (100 mg, 0.2 mmol) in DMF (5 mL) were added 5-(4,4-difluoropiperidin-3-yl)pyrazin-2(1H)-one (52 mg, 0.2 mmol), lithium trifluoromethanesulfonate (158 mg, 1.0 mmol) and DIEA (131 mg, 1.0 mmol) dropwise at 0° C. The resulting mixture was stirred at 0° C. for 5 days under nitrogen atmosphere. The reaction mixture was directly purified by reversed-phase flash chromatography (conditions: column, C18 silica gel; mobile phase, ACN in water (0.1% formic acid), 0% to 100% gradient in 25 min; detector, UV 254 nm) to give (2S)-2-(4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide (40 mg, 39% yield) as a yellow solid.

LCMS (ESI) [M+H]⁺: 505.

### Step 3: (S)-2-((R)-4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide and (S)-2-((S)-4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide

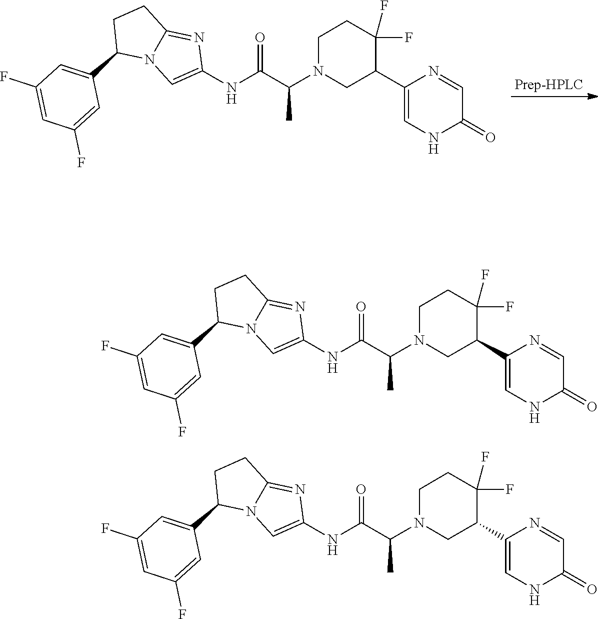 [SMILES: C[C@@H](C(=O)Nc1cn2c(n1)CC[C@@H]2c1cc(F)cc(F)c1)N1CCC(F)(F)C(c2c[nH]c(=O)cn2)C1 ; C[C@@H](C(=O)Nc1cn2c(n1)CC[C@@H]2c1cc(F)cc(F)c1)N1CCC(F)(F)[C@@H](c2c[nH]c(=O)cn2)C1 ; C[C@@H](C(=O)Nc1cn2c(n1)CC[C@@H]2c1cc(F)cc(F)c1)N1CCC(F)(F)[C@H](c2c[nH]c(=O)cn2)C1]

(2S)-2-(4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide (40 mg) was separated by Prep-HPLC with the following conditions (Column: Xselect CSH Prep C18, 30*150 mm 5 μm; Mobile Phase A: Water (0.1% FA), Mobile Phase B: ACN; Flow rate: 60 mL/min; Gradient: 2% to 23% B in 14 min; Wave Length: 254 nm/220 nm; RT1 (min): 10.27; RT2 (min): 13.98) to afford first peak (S)-2-((R)-4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide (Compound 527) (Method AK, Peak 2, 1.75 min; 4.4 mg, 100% d.e.) as a white solid and second peak (S)-2-((S)-4,4-difluoro-3-(5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide (Compound 528) (Method AK, Peak 1, 1.55 min; 9.2 mg, 100% d.e.) as a white solid.

TABLE 43

| Compound# | Characterization Data |
|---|---|
| 527 | LCMS (ESI) [M + H]⁺: 505.2 |
| | ¹H NMR (300 MHz, DMSO-d₆) δ 12.25 (bs, 1H), 10.25 (s, 1H), 8.09 (s, 1H), 7.47 (s, 1H), |
| | 7.25-7.22 (m, 1H), 7.19-6.97 (m, 3H), 5.50-5.29 (m, 1H), 3.52-3.47 (m, 2H), 2.96- |
| | 2.93 (m, 3H), 2.83-2.69 (m, 3H), 2.64-2.61 (m, 1H), 2.43-2.38 (m, 1H), 2.28-1.92 (m, |
| | 2H), 1.17-1.12 (m, 3H). |
| 528 | LCMS (ESI) [M + H]⁺: 505.2 |
| | ¹H NMR (400 MHz, DMSO-d₆) δ 12.25 (bs, 1H), 10.13 (s, 1H), 8.03 (s, 1H), 7.41-7.38 (m, |
| | 1H), 7.24-7.21 (m, 1H), 6.96-6.93 (m, 3H), 5.75-5.36 (m, 1H), 3.52-3.51 (m, 2H), |
| | 3.07-2.67 (m, 7H), 2.35-2.33 (m, 1H), 2.12-1.95 (m, 2H), 1.17-1.12 (m, 3H). |

### Example 69: (2S)-2-(4,4-difluoro-3-(5-((methylamino)methyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

### Step 1: benzyl 4,4-difluoro-3-(6-methoxy-5-((methylamino)methyl)pyridin-3-yl)piperidine-1-carboxylate

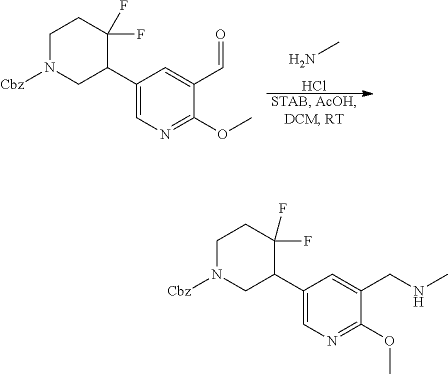

To a mixture of benzyl 4,4-difluoro-3-(5-formyl-6-methoxypyridin-3-yl)piperidine-1-carboxylate (400 mg, 1.0 mmol) in DCM (8 mL) were added methylamine hydrochloride (83 mg, 1.2 mmol) and AcOH (6 mg, 0.1 mmol) dropwise at 0° C. under nitrogen atmosphere. The resulting mixture was stirred at room temperature for 1 h under nitrogen atmosphere. To the above mixture was added STAB (326 mg, 1.5 mmol) in portions over 5 min at 0° C. The resulting mixture was stirred at room temperature for additional 2 h. The reaction was quenched with water (10 mL) at 0° C. The aqueous layer was extracted with EtOAc (3×15 mL). The combined organic layers were dried over anhydrous Na₂SO₄, filtered and concentrated under reduced pressure. The crude residue was purified by silica gel column chromatography, eluted with PE/EA (1:1) to afford benzyl 4,4-difluoro-3-(6-methoxy-5-((methylamino)methyl)pyridin-3-yl)piperidine-1-carboxylate (200 mg, 48% yield) as a white solid.

LCMS (ESI) [M+H]⁺: 406.

### Step 2: benzyl 3-(5-(((tert-butoxycarbonyl)(methyl)amino)methyl)-6-methoxypyridin-3-yl)-4,4-difluoropiperidine-1-carboxylate

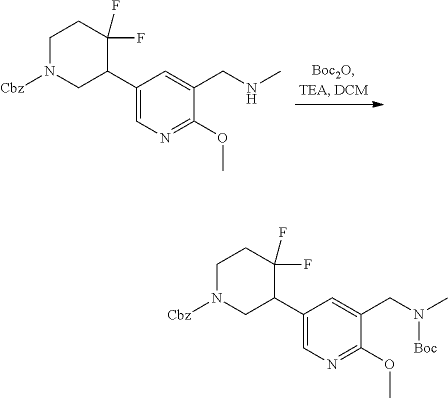 [SMILES: CNCc1cc(C2CN(C(=O)OCc3ccccc3)CCC2(F)F)cnc1OC ; COc1ncc(C2CN(C(=O)OCc3ccccc3)CCC2(F)F)cc1CN(C)C(=O)OC(C)(C)C]

To a mixture of benzyl 4,4-difluoro-3-(6-methoxy-5-((methylamino)methyl)pyridin-3-yl)piperidine-1-carboxylate (200 mg, 0.5 mmol) in DCM (6 mL) were added TEA (150 mg, 1.5 mmol) and Boc₂O (161 mg, 0.7 mmol) dropwise at 0° C. under nitrogen atmosphere. The resulting mixture was stirred at room temperature for 2 h under nitrogen atmosphere. The resulting mixture was concentrated under vacuum. The crude residue was directly purified by silica gel column chromatography, eluted with PE/EA (4:1) to afford benzyl 3-(5-(((tert-butoxycarbonyl)(methyl)amino)methyl)-6-methoxypyridin-3-yl)-4,4-difluoropiperidine-1-carboxylate (200 mg, 80% yield) as a white solid.

LCMS (ESI) [M+H]⁺: 506.

### Step 3: tert-butyl ((5-(4,4-difluoropiperidin-3-yl)-2-methoxypyridin-3-yl)methyl)(methyl)carbamate

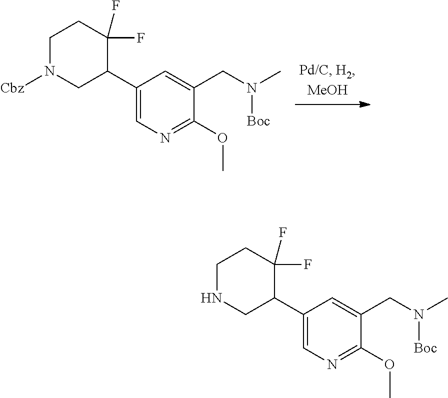 [SMILES: COc1ncc(C2CN(C(=O)OCc3ccccc3)CCC2(F)F)cc1CN(C)C(=O)OC(C)(C)C ; COc1ncc(C2CNCCC2(F)F)cc1CN(C)C(=O)OC(C)(C)C]

A solution of benzyl 3-(5-(((tert-butoxycarbonyl)(methyl)amino)methyl)-6-methoxypyridin-3-yl)-4,4-difluoropiperidine-1-carboxylate (200 mg, 0.4 mmol) and 10% Pd/C (42 mg, anhydrous) in MeOH (8 mL) was stirred at room temperature for 2 h under hydrogen atmosphere. The resulting mixture was filtered through celite. The filter cake was washed with MeOH (3×10 mL). The filtrate was concentrated under reduced pressure. The crude residue was purified by reversed-phase flash chromatography (conditions: column, C18 silica gel; mobile phase, ACN in water (0.1% formic acid), 0% to 100% gradient in 20 min; detector, UV 254 nm) to give tert-butyl ((5-(4,4-difluoropiperidin-3-yl)-2-methoxypyridin-3-yl)methyl)(methyl)carbamate (120 mg, 82% yield) as a colorless oil.

LCMS (ESI) [M+H]⁺: 372.

### Step 4: tert-butyl ((5-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-methoxypyridin-3-yl)methyl)(methyl)carbamate

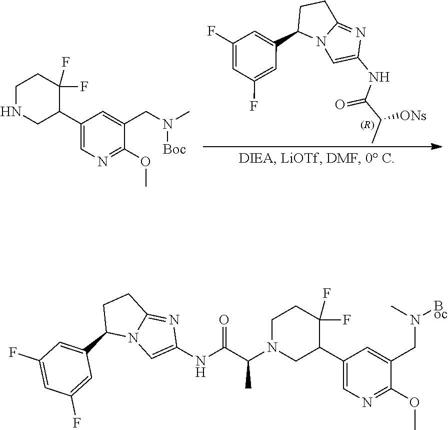

A solution of tert-butyl ((5-(4,4-difluoropiperidin-3-yl)-2-methoxypyridin-3-yl)methyl)(methyl)carbamate (110 mg, 0.3 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (175 mg, 0.4 mmol), DIEA (96 mg, 0.7 mmol) and lithium trifluoromethanesulfonate (120 mg, 0.8 mmol) in DMF (5 mL) was stirred at 0° C. for 5 days under nitrogen atmosphere. The reaction mixture was directly purified by reversed-phase flash chromatography (conditions: column, C18 silica gel; mobile phase, ACN in water (0.1% formic acid), 0% to 100% gradient in 20 min; detector, UV 254 nm) to give tert-butyl ((5-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-methoxypyridin-3-yl)methyl)(methyl)carbamate (50 mg, 26% yield) as a yellow solid.

LCMS (ESI) [M+H]⁺: 661.

### Step 5: (2S)-2-(4,4-difluoro-3-(5-((methylamino)methyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

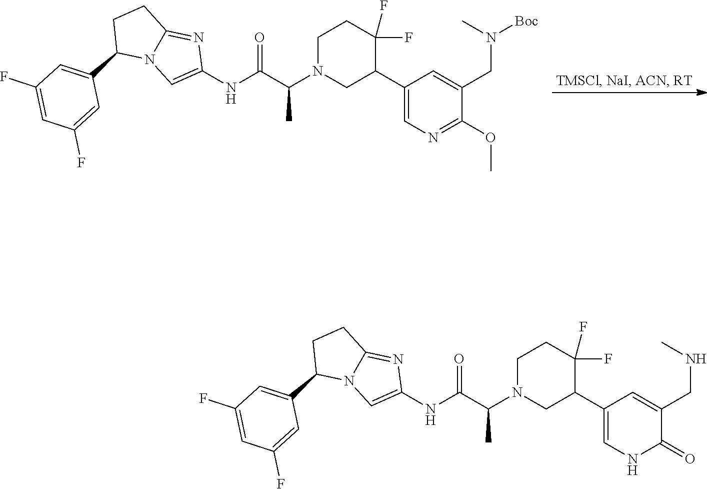 [SMILES: COc1ncc(C2CN([C@@H](C)C(=O)Nc3cn4c(n3)CC[C@@H]4c3cc(F)cc(F)c3)CCC2(F)F)cc1CN(C)C(=O)OC(C)(C)C ; CNCc1cc(C2CN([C@@H](C)C(=O)Nc3cn4c(n3)CC[C@@H]4c3cc(F)cc(F)c3)CCC2(F)F)c[nH]c1=O]

To a mixture of tert-butyl ((5-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-methoxypyridin-3-yl)methyl)(methyl)carbamate (50 mg, 80.0 μmol) in ACN (3 mL) were added NaI (57 mg, 0.4 mmol) and TMSCl (41 mg, 0.4 mmol) dropwise over 1 min at 0° C. The resulting mixture was stirred at room temperature for an additional 3 h. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 22 min; detector, UV 254 nm) to give (2S)-2-(4,4-difluoro-3-(5-((methylamino)methyl)-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 529) (7.8 mg, 17% yield) as a white solid.

TABLE 44

| Compound# | Characterization Data |
|---|---|
| 529 | LCMS (ESI) [M + H]⁺: 547.4 |
| | ¹H NMR (400 MHz, DMSO-d₆) δ 10.10 (s, 1H), 7.48 (s, 1H), 7.33-7.18 (m, 2H), 6.96-6.94 |
| | (m, 3H), 5.38 (dd, J = 6.6 Hz, 1H), 3.57 (s, 3H), 3.46-3.32 (m, 2H), 2.98-2.72 (m, 5H), |
| | 2.68-2.57 (m, 1H), 2.39-2.33 (m, 5H), 2.12-1.88 (m, 2H), 1.16-1.15 (m, 3H). |

### Example 70: (2S)-2-((S)-4,4-difluoro-3-(6-oxo-1,6-dihydropyridin-3-yl)piperidin-1-yl)-N-(4-(3,5-difluorophenyl)-5,6-dihydro-4H-pyrrolo[1,2-b]pyrazol-2-yl)propanamide

### Step 1: 3,5-dibromo-1-(tetrahydro-2H-pyran-2-yl)-1H-pyrazole

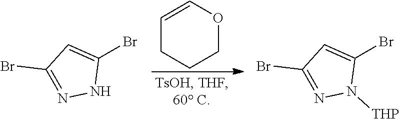

A solution of 3,5-dibromo-1H-pyrazole (5.00 g, 22.1 mmol), 3,4-dihydro-2H-pyran (2.23 g, 26.6 mmol) and TsOH (381 mg, 2.2 mmol) in THF (60 mL) was stirred at 60° C. for 2 h under nitrogen atmosphere. The mixture was allowed to cool down to room temperature. The resulting mixture was concentrated under reduced pressure. The crude residue was directly purified by silica gel column chromatography, eluted with PE/EA (1:1) to afford 3,5-dibromo-1-(tetrahydro-2H-pyran-2-yl)-1H-pyrazole (5.30 g, 77% yield) as a white solid.

LCMS (ESI) [M+H]⁺: 309.

### Step 2: 3-bromo-5-(3,5-difluorobenzyl)-1-(tetrahydro-2H-pyran-2-yl)-1H-pyrazole

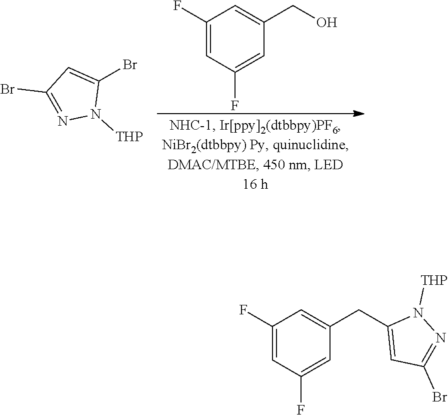

An oven-dried 50 mL vial was charged with (3,5-difluorophenyl)methanol (1.22 g, 8.5 mmol), 5,7-di-tert-butyl-3-phenylbenzo[d]oxazol-3-ium tetrafluoroborate (NHC-1) (3.06 g, 7.7 mmol) and a magnetic stir bar. After the vial was vacuumed and refilled with nitrogen gas twice, MTBE (37 mL) was added and the reaction stirred at 0° C. for 10 min. Then, a pyridine solution (0.61 g, 7.7 mmol in 0.5 mL 2-methoxy-2-methylpropane) was added dropwise at 0° C. over the course of 2 min. The resulting solution stirred at room temperature for 10 min. A white solid precipitated out during this time. In a separate oven-dried 40 mL vial was charged with [Ir(ppy)₂(dtbbpy)]PF₆ (0.07 g, 73.0 μmol), [4,4′-Bis(tert-butyl)-2,2′-bipyridine]nickel dibromide (0.18 g, 0.4 mmol), 1-azabicyclo[2.2.2]octane (0.94 g, 8.5 mmol), 3,5-dibromo-1-(tetrahydro-2H-pyran-2-yl)-1H-pyrazole (1.50 g, 4.8 mmol) and a magnetic stir bar. N,N-Dimethylacetamide (37.5 mL) was added to this vial under an atmosphere of nitrogen. The MTBE suspension was transferred to a 50 mL syringe under air. Then, a syringe filter and new needle were installed on the syringe, before the MTBE solution was injected through the syringe filter into the dimethylacetamide solution. The reaction mixture was sparged with nitrogen for 15 minutes before sealing with parafilm. The vial was stirred at 1500 rpm stir rate and irradiated under 450 nm LED modules at 100% light intensity for 2 hours. The reaction was quenched with water. The resulting mixture was extracted with EtOAc (3×90 mL). The combined organic layers were washed with water (3×95 mL), dried over anhydrous Na₂SO₄, filtered and concentrated under reduced pressure. The crude residue was purified by reversed-phase flash chromatography (conditions: column, C18 silica gel; mobile phase, ACN in water (0.1% formic acid), 0% to 100% gradient in 25 min; detector, UV 254 nm) to give 3-bromo-5-(3,5-difluorobenzyl)-1-(tetrahydro-2H-pyran-2-yl)-1H-pyrazole (800 mg, 46% yield) as a light yellow oil.

LCMS (ESI) [M+H]⁺: 357.

### Step 3: 3-bromo-5-(3-((tert-butyldimethylsilyl)oxy)-1-(3,5-difluorophenyl)propyl)-1-(tetrahydro-2H-pyran-2-yl)-1H-pyrazole

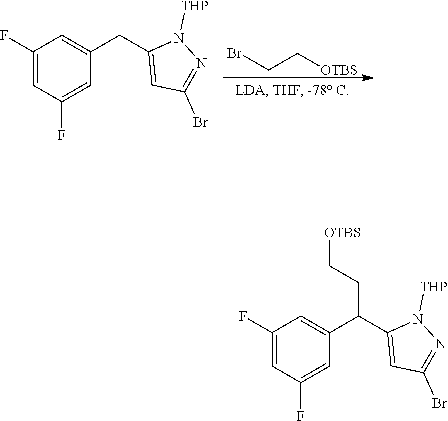

LDA (1.6 mL, 2.4 mmol) was added dropwise over 10 min to a solution of 3-bromo-5-(3,5-difluorobenzyl)-1-(tetrahydro-2H-pyran-2-yl)-1H-pyrazole (700 mg, 2.0 mmol) in THF (12 mL) at −78° C. under nitrogen atmosphere. The resulting mixture was stirred at −78° C. for 15 min. To the above mixture was added (2-bromoethoxy)(tert-butyl)dimethylsilane (562 mg, 2.4 mmol) dropwise over 10 min at −78° C. The resulting mixture was stirred at −78° C. for additional 1 h. The reaction mixture was quenched with saturated aqueous NH₄Cl solution (15 mL) at 0° C. The aqueous layer was extracted with EtOAc (3×20 mL). The combined organic layers were dried over anhydrous Na₂SO₄, filtered and concentrated under reduced pressure. The crude residue was purified by silica gel column chromatography, eluted with PE/EA (2:1) to afford 3-bromo-5-(3-((tert-butyldimethylsilyl)oxy)-1-(3,5-difluorophenyl)propyl)-1-(tetrahydro-2H-pyran-2-yl)-1H-pyrazole (600 mg, 59% yield) as a colorless oil.

LCMS (ESI) [M+H]⁺: 515.

### Step 4: 3-(3-bromo-1H-pyrazol-5-yl)-3-(3,5-difluorophenyl)propan-1-ol

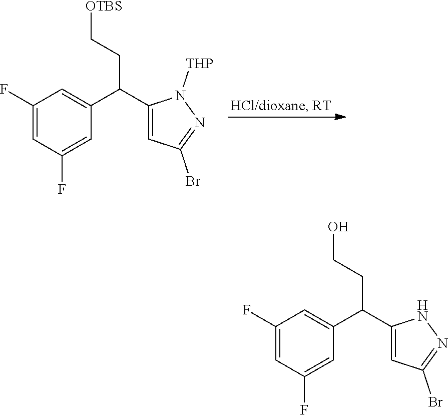

A solution of 3-bromo-5-(3-((tert-butyldimethylsilyl)oxy)-1-(3,5-difluorophenyl)propyl)-1-(tetrahydro-2H-pyran-2-yl)-1H-pyrazole (600 mg, 1.2 mmol) in 4M HCl in 1,4-dioxane (8 mL) was stirred for 2 h at room temperature under an ambient atmosphere. The resulting mixture was concentrated under reduced pressure. The crude residue was directly purified by reversed-phase flash (conditions: column, C18 silica gel; mobile phase, ACN in water (0.1% formic acid), 0% to 100% gradient in 20 min; detector, UV 254 nm) to give 3-(3-bromo-1H-pyrazol-5-yl)-3-(3,5-difluorophenyl)propan-1-ol (210 mg, 57% yield) as a colorless oil.

LCMS (ESI) [M+H]⁺: 317.

### Step 5: 2-bromo-4-(3,5-difluorophenyl)-5,6-dihydro-4H-pyrrolo[1,2-b]pyrazole

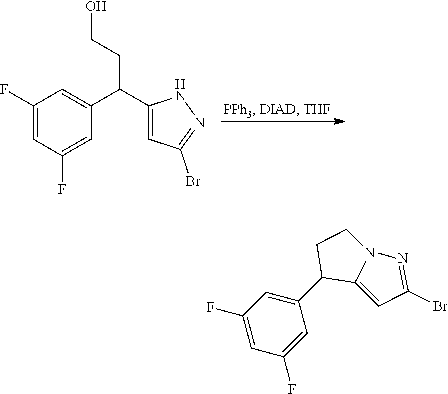

A solution of 3-(3-bromo-1H-pyrazol-5-yl)-3-(3,5-difluorophenyl)propan-1-ol (200 mg, 0.6 mmol) and PPh₃ (198 mg, 0.8 mmol) in THF (6 mL) was stirred at 0° C. for 10 min under nitrogen atmosphere. To the above mixture was added DIAD (153 mg, 0.8 mmol) dropwise over 5 min at 0° C. The resulting mixture was stirred at room temperature for additional 2 h. The reaction mixture was quenched with water (10 mL) at 0° C. The resulting mixture was extracted with EtOAc (3×12 mL). The combined organic layers were washed with brine, dried over anhydrous Na₂SO₄, filtered and concentrated under reduced pressure. The crude residue was purified by reversed-phase flash chromatography (conditions: column, C18 silica gel; mobile phase, ACN in water (0.1% formic acid), 0% to 100% gradient in 20 min; detector, UV 254 nm) to give 2-bromo-4-(3,5-difluorophenyl)-5,6-dihydro-4H-pyrrolo[1,2-b]pyrazole (120 mg, 64% yield) as a light yellow oil.

LCMS (ESI) [M+H]⁺: 299.

### Step 6: (2R)—N-(4-(3,5-difluorophenyl)-5,6-dihydro-4H-pyrrolo[1,2-b]pyrazol-2-yl)-2-hydroxypropanamide

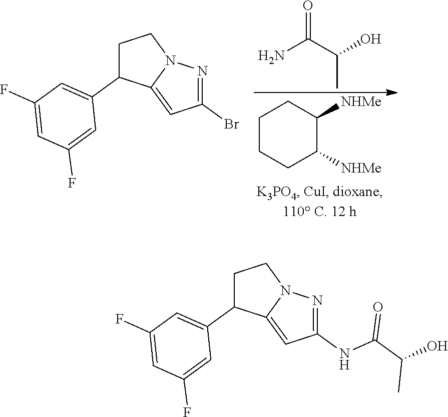

A solution of 2-bromo-4-(3,5-difluorophenyl)-5,6-dihydro-4H-pyrrolo[1,2-b]pyrazole (120 mg, 0.4 mmol), (R)-2-hydroxypropanamide (42 mg, 0.5 mmol), (1R,2R)—N1,N2-dimethylcyclohexane-1,2-diamine (22 mg, 0.2 mmol), CuI (15 mg, 80.0 μmol) and K₃PO₄ (170 mg, 0.8 mmol) in dioxane (6 mL) was stirred at 110° C. for 12 h under nitrogen atmosphere. The mixture was allowed to cool down to room temperature. The reaction mixture was diluted with water (10 mL) at 0° C. The resulting mixture was extracted with EtOAc (3×12 mL). The combined organic layers were dried over anhydrous Na₂SO₄, filtered and concentrated under reduced pressure. The crude residue was purified by reversed-phase flash chromatography (conditions: column, C18 silica gel; mobile phase, ACN in water (0.1% formic acid), 0% to 100% gradient in 20 min; detector, UV 254 nm) to give (2R)—N-(4-(3,5-difluorophenyl)-5,6-dihydro-4H-pyrrolo[1,2-b]pyrazol-2-yl)-2-hydroxypropanamide (100 mg, 81% yield) as a light yellow solid.

LCMS (ESI) [M+H]⁺: 308.

### Step 7: (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate

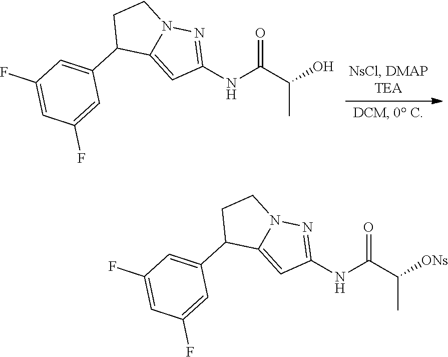

A solution of (2R)—N-(4-(3,5-difluorophenyl)-5,6-dihydro-4H-pyrrolo[1,2-b]pyrazol-2-yl)-2-hydroxypropanamide (100 mg, 0.3 mmol), DMAP (8 mg, 60.0 μmol) and 2-nitrobenzenesulfonyl chloride (28 mg, 0.1 mmol) in DCM (6 mL) was stirred at 0° C. for 3 min under nitrogen atmosphere. Triethylamine (98 mg, 1.0 mmol) was added dropwise over 5 min at 0° C. The resulting mixture was stirred at 0° C. for an additional 5 h. The reaction mixture was diluted with ice water (10 mL) at 0° C. The resulting mixture was extracted with CH₂Cl₂ (3×12 mL). The combined organic layers were dried over anhydrous Na₂SO₄, filtered and concentrated under reduced pressure. The crude residue was purified by silica gel column chromatography, eluted with CH₂Cl₂/EA (1:1) to afford (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (70 mg, 44% yield) as a yellow solid.

LCMS (ESI) [M+H]⁺: 493.

### Step 8: (2S)-2-((S)-4,4-difluoro-3-(6-oxo-1,6-dihydropyridin-3-yl)piperidin-1-yl)-N-(4-(3,5-difluorophenyl)-5,6-dihydro-4H-pyrrolo[1,2-b]pyrazol-2-yl)propanamide

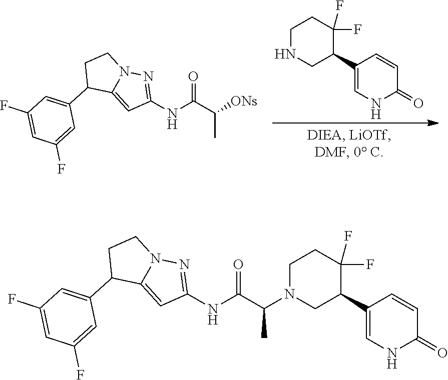

A solution of (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (50 mg, 0.1 mmol), (S)-5-(4,4-difluoropiperidin-3-yl)pyridin-2(1H)-one (26 mg, 0.1 mmol), DIEA (32 mg, 0.3 mmol) and lithium trifluoromethanesulfonate (41 mg, 0.3 mmol) in DMF (2 mL) was stirred at 0° C. for 3 days under nitrogen atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to give (2S)-2-((S)-4,4-difluoro-3-(6-oxo-1,6-dihydropyridin-3-yl)piperidin-1-yl)-N-(4-(3,5-difluorophenyl)-5,6-dihydro-4H-pyrrolo[1,2-b]pyrazol-2-yl)propanamide (30 mg, 57% yield) as a light yellow solid.

LCMS (ESI) [M+H]⁺: 504.

### Step 9: (S)-2-((S)-4,4-difluoro-3-(6-oxo-1,6-dihydropyridin-3-yl)piperidin-1-yl)-N—((R)-4-(3,5-difluorophenyl)-5,6-dihydro-4H-pyrrolo[1,2-b]pyrazol-2-yl)propanamide and (S)-2-((S)-4,4-difluoro-3-(6-oxo-1,6-dihydropyridin-3-yl)piperidin-1-yl)-N—((S)-4-(3,5-difluorophenyl)-5,6-dihydro-4H-pyrrolo[1,2-b]pyrazol-2-yl)propanamide

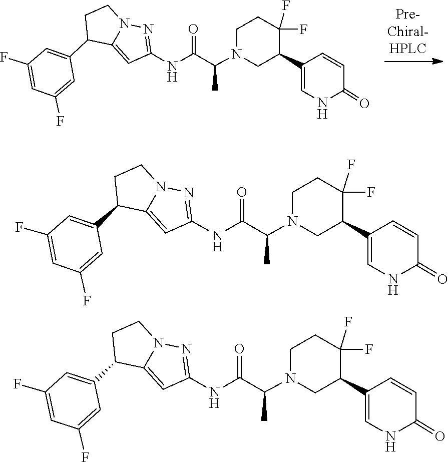 [SMILES: C[C@@H](C(=O)Nc1cc2n(n1)CCC2c1cc(F)cc(F)c1)N1CCC(F)(F)[C@@H](c2ccc(=O)[nH]c2)C1 ; C[C@@H](C(=O)Nc1cc2n(n1)CC[C@@H]2c1cc(F)cc(F)c1)N1CCC(F)(F)[C@@H](c2ccc(=O)[nH]c2)C1 ; C[C@@H](C(=O)Nc1cc2n(n1)CC[C@H]2c1cc(F)cc(F)c1)N1CCC(F)(F)[C@@H](c2ccc(=O)[nH]c2)C1]

(2S)-2-((S)-4,4-difluoro-3-(6-oxo-1,6-dihydropyridin-3-yl)piperidin-1-yl)-N-(4-(3,5-difluorophenyl)-5,6-dihydro-4H-pyrrolo[1,2-b]pyrazol-2-yl)propanamide (30 mg) was separated by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 50% B; Wave Length: 206/232 nm; RT1 (min): 11.5; RT2 (min): 16.5) to afford first peak (S)-2-((S)-4,4-difluoro-3-(6-oxo-1,6-dihydropyridin-3-yl)piperidin-1-yl)-N—((R)-4-(3,5-difluorophenyl)-5,6-dihydro-4H-pyrrolo[1,2-b]pyrazol-2-yl)propanamide Compound 530) (Method Y, Peak 1, 1.39 min; 12.2 mg, 100% d.e.) as a white solid and second peak (S)-2-((S)-4,4-difluoro-3-(6-oxo-1,6-dihydropyridin-3-yl)piperidin-1-yl)-N—((S)-4-(3,5-difluorophenyl)-5,6-dihydro-4H-pyrrolo[1,2-b]pyrazol-2-yl)propanamide (Compound 531) (Method Y, Peak 2, 1.87 min; 10.5 mg, 100% d.e.) as a white solid.

TABLE 45

| Compound# | Characterization Data |
|---|---|
| 530 | LCMS (ESI) [M + H]⁺: 504.3 |
| | ¹H NMR (400 MHz, DMSO-d₆) δ 11.48 (bs, 1H), 10.17 (s, 1H), 7.41-7.40 (m, 1H), 7.28 (d, |
| | J = 2.6 Hz, 1H), 7.13-7.12 (m, 1H), 7.06-6.97 (m, 2H), 6.28 (d, J = 9.5 Hz, 1H), 6.24 (s, |
| | 1H), 4.55-4.45 (m, 1H), 4.26-4.17 (m, 1H), 4.09-3.92 (m, 1H), 3.54-3.47 (m, 1H), |
| | 3.24-3.09 (m, 1H), 3.08-2.97 (m, 1H), 2.89-2.72 (m, 3H), 2.48-2.38 (m, 2H), |
| | 2.13-1.91 (m, 2H), 1.17 (d, J = 7.0 Hz, 3H). |
| 531 | LCMS (ESI) [M + H]⁺: 504.3 |
| | ¹H NMR (400 MHz, DMSO-d₆) δ 11.52 (bs, 1H), 10.20 (s, 1H), 7.42-7.35 (m, 1H), 7.27 (s, |
| | 1H), 7.18-7.09 (m, 1H), 7.04-6.96 (m, 2H), 6.21-6.30 (m, 2H), 4.53-4.47 (m, 1H), |
| | 4.23-4.15 (m, 1H), 4.08-3.92 (m, 1H), 3.52-3.47 (m, 1H), 3.24-3.09 (m, 1H), |
| | 3.02-2.92 (m, 1H), 2.90-2.82 (m, 2H), 2.81-2.75 (m, 1H), 2.43-2.51 (m, 2H), |
| | 2.10-1.92 (m, 2H), 1.17 (d, J = 6.9 Hz, 3H). |

### Example 71: (2S)-2-(4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

### Step 1: tert-butyl 4,4-difluoro-3-(6-oxo-1,6-dihydropyridin-3-yl)piperidine-1-carboxylate

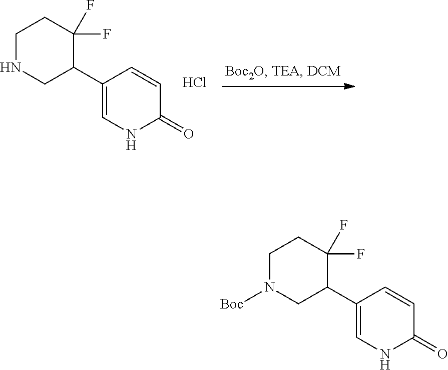 [SMILES: O=c1ccc(C2CNCCC2(F)F)c[nH]1 ; Cl ; CC(C)(C)OC(=O)N1CCC(F)(F)C(c2ccc(=O)[nH]c2)C1]

To a mixture of 5-(4,4-difluoropiperidin-3-yl)pyridin-2(1H)-one hydrochloride (500 mg, 2.3 mmol) and Et₃N (0.71 g, 7.0 mmol) in DCM (10 mL) was added Boc₂O (1.53 g, 7.0 mmol) dropwise at 0° C. under nitrogen atmosphere. The reaction was stirred at room temperature for 2 h. The resulting mixture was concentrated under reduced pressure. The crude residue was directly purified by silica gel column chromatography, eluted with PE/EA (3:2) to afford tert-butyl 4,4-difluoro-3-(6-oxo-1,6-dihydropyridin-3-yl)piperidine-1-carboxylate (600 mg, 82% yield) as a yellow solid.

LCMS (ESI) [M+H]⁺: 315.

### Step 2: tert-butyl 4,4-difluoro-3-(1-methyl-6-oxo-1,6-dihydropyridin-3-yl)piperidine-1-carboxylate

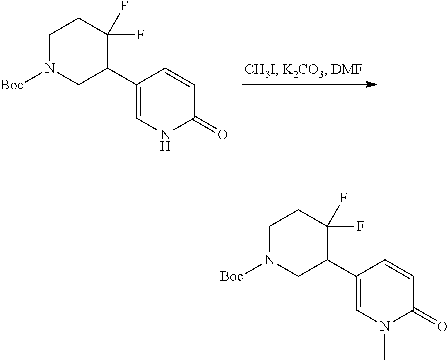 [SMILES: CC(C)(C)OC(=O)N1CCC(F)(F)C(c2ccc(=O)[nH]c2)C1 ; Cn1cc(C2CN(C(=O)OC(C)(C)C)CCC2(F)F)ccc1=O]

To a mixture of tert-butyl 4,4-difluoro-3-(6-oxo-1,6-dihydropyridin-3-yl)piperidine-1-carboxylate (600 mg, 1.9 mmol) and K₂CO₃ (1.32 g, 9.5 mmol) in DMF (8 mL) was added CH₃I (0.81 g, 5.7 mmol) dropwise at 0° C. under nitrogen atmosphere. The reaction was stirred at room temperature for 2 h. The reaction was quenched with water at 0° C. The resulting mixture was extracted with EtOAc (3×10 mL). The combined organic layers were washed with brine, dried over anhydrous Na₂SO₄, filtered and concentrated under reduced pressure. The crude residue was purified by silica gel column chromatography, eluted with PE/EA (1:1) to afford tert-butyl 4,4-difluoro-3-(1-methyl-6-oxo-1,6-dihydropyridin-3-yl)piperidine-1-carboxylate (240 mg, 38% yield) as a yellow solid.

LCMS (ESI) [M+H]⁺: 329.

### Step 3: 5-(4,4-difluoropiperidin-3-yl)-1-methylpyridin-2(1H)-one

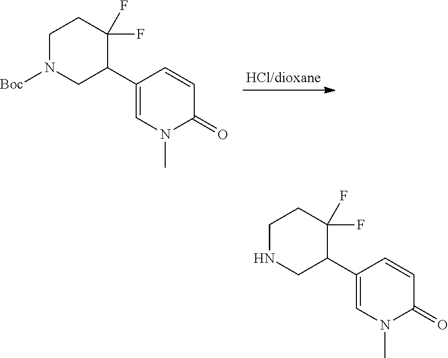 [SMILES: Cn1cc(C2CN(C(=O)OC(C)(C)C)CCC2(F)F)ccc1=O ; Cn1cc(C2CNCCC2(F)F)ccc1=O]

A solution of tert-butyl 4,4-difluoro-3-(1-methyl-6-oxo-1,6-dihydropyridin-3-yl)piperidine-1-carboxylate (240 mg, 0.7 mmol) in 4 M HCl in 1,4-dioxane (10 mL) was stirred for 1 hour at room temperature under an ambient atmosphere. The resulting mixture was concentrated under reduced pressure to give 5-(4,4-difluoropiperidin-3-yl)-1-methylpyridin-2(1H)-one (200 mg, crude) as a yellow solid, which was used directly in the next step without further purification.

LCMS (ESI) [M+H]⁺: 229.

### Step 4: (2S)-2-(4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

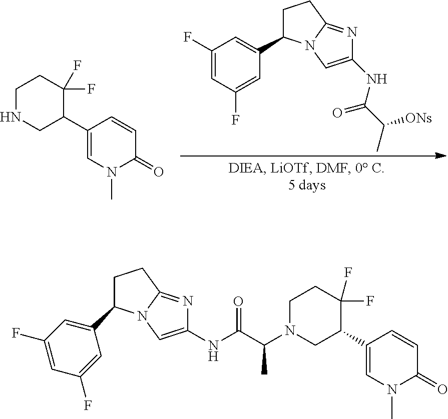

To a mixture of (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (150 mg, 0.3 mmol) and 5-(4,4-difluoropiperidin-3-yl)-1-methylpyridin-2(1H)-one (83 mg, 0.3 mmol) in DMF (6 mL) were added lithium trifluoromethanesulfonate (238 mg, 1.5 mmol) and DIEA (197 mg, 1.5 mmol) dropwise at 0° C. The reaction was stirred at 0° C. for 5 days under nitrogen atmosphere. The reaction mixture was directly purified by reversed-phase flash chromatography (conditions: column, C18 silica gel; mobile phase A: Water (10 mmol/L NH₄HCO₃), mobile phase B: ACN; Flow rate: 40 mL/min; gradient: 45% B to 55% B in 15 min; Wave Length: 254 nm/220 nm) to afford (2S)-2-(4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (70 mg, 44% yield) as a yellow solid.

LCMS (ESI) [M+H]⁺: 518.

### Step 5: (S)-2-((S)-4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide and (S)-2-((R)-4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide

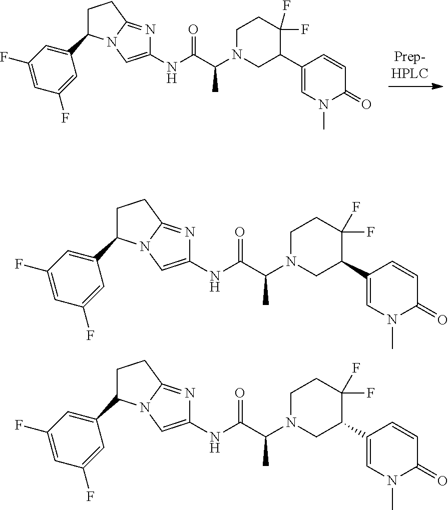

(2S)-2-(4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (70 mg) was separated by Prep-HPLC (conditions: Column, Xselect CSH Prep C18, 30*150 mm 5 μm; Mobile Phase A: Water (0.1% FA), Mobile Phase B: MeOH; Flow rate: 60 mL/min; Gradient: 31% to 50% B in 15 min; Wave Length: 254 nm/220 nm; RT1 (min): 13.70; RT2 (min): 16.62 min) to afford first peak (S)-2-((S)-4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 532) (Method AL, Peak 1, 2.22 min; 12.4 mg, 100% d.e.) as a white solid and second peak (S)-2-((R)-4,4-difluoro-3-(1-methyl-6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide (Compound 533) (Method AL, Peak 2, 2.42 min; 7.5 mg, 97% d.e.) as a white solid.

TABLE 46

| Compound# | Characterization Data |
|---|---|
| 532 | LCMS (ESI) [M + H]⁺: 518.2 |
| | ¹H NMR (400 MHz, DMSO-d₆) δ 10.11 (s, 1H), 7.71-7.62 (m, 1H), 7.41-7.38 (m, 1H), |
| | 7.22-7.18 (m, 1H), 7.03-6.87 (m, 3H), 6.33 (d, J = 9.4 Hz, 1H), 5.41-5.38 (m, 1H), |
| | 3.52-3.45 (m, 1H), 3.40 (s, 3H), 3.18-3.03 (m, 1H), 3.02-2.73 (m, 5H), 2.54-2.64 (m, 2H), |
| | 2.43-2.31 (m, 1H), 2.18-1.93 (m, 2H), 1.17 (d, J = 6.9 Hz, 3H). |
| 533 | LCMS (ESI) [M + H]⁺: 518.2 |
| | ¹H NMR (400 MHz, DMSO-d₆) δ 10.11 (s, 1H), 7.68-7.62 (m, 1H), 7.42-7.31 (m, 1H), |
| | 7.26-7.18 (m, 1H), 6.99-6.91 (m, 3H), 6.34 (d, J = 9.4 Hz, 1H), 5.35-5.42 (m, 1H), |
| | 3.52-3.42 (m, 4H), 3.21-3.07 (m, 1H), 2.99-2.84 (m, 4H), 2.82-2.73 (m, 2H), |
| | 2.50-2.30 (m, 2H), 2.18-1.92 (m, 2H), 1.16 (d, J = 6.9 Hz, 3H). |

### Example 72: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide

### Step 1: tert-butyl 3-[2-(1,3-dioxolan-2-yl)pyridin-4-yl]-4,4-difluoropiperidine-1-carboxylate

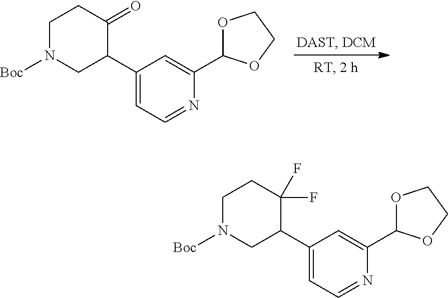 [SMILES: CC(C)(C)OC(=O)N1CCC(=O)C(c2ccnc(C3OCCO3)c2)C1 ; CC(C)(C)OC(=O)N1CCC(F)(F)C(c2ccnc(C3OCCO3)c2)C1]

To a stirred mixture of tert-butyl 3-[2-(1,3-dioxolan-2-yl) pyridin-4-yl]-4-oxopiperidine-1-carboxylate (3.50 g, 10.0 mmol) in DCM (100 mL) was added DAST (3.22 g, 20.0 mmol) dropwise at 0° C. under nitrogen atmosphere. The resulting mixture was stirred at room temperature for additional 2 h. The reaction was quenched with sat. NaHCO₃ (aq.) at 0° C. The resulting mixture was extracted with CH₂Cl₂ (3×200 mL). The combined organic layers were dried over anhydrous Na₂SO₄, filtered and concentrated under reduced pressure. The crude residue was purified by silica gel column chromatography, eluted with PE/EA (1:1) to afford tert-butyl 3-[2-(1,3-dioxolan-2-yl)pyridin-4-yl]-4,4-difluoropiperidine-1-carboxylate (2.7 g, 72% yield) as a light yellow oil.

LCMS [M+H]⁺: 371.

### Step 2: tert-butyl 4,4-difluoro-3-(2-formylpyridin-4-yl) piperidine-1-carboxylate

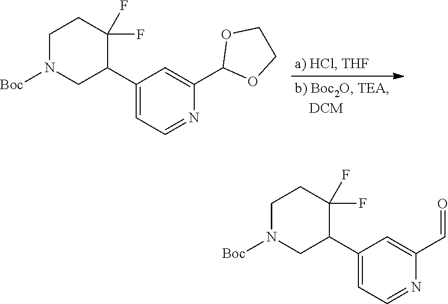 [SMILES: CC(C)(C)OC(=O)N1CCC(F)(F)C(c2ccnc(C3OCCO3)c2)C1 ; CC(C)(C)OC(=O)N1CCC(F)(F)C(c2ccnc(C=O)c2)C1]

A solution of tert-butyl 3-[2-(1,3-dioxolan-2-yl)pyridin-4-yl]-4,4-difluoropiperidine-1-carboxylate (2.7 g, 7.2 mmol) in THF (45 mL) and HCl (aq) (45 mL, 3 M) was stirred at 60° C. for 1 h under nitrogen atmosphere. The resulting mixture was concentrated under reduced pressure. The crude residue was dissolved in DCM (30 mL). Then, Boc₂O (3.18 g, 14.5 mmol) and TEA (7.37 g, 72.8 mmol) were added in portions at 0° C. The resulting mixture was stirred at room temperature for an additional 2 h. The reaction was diluted with water. The resulting mixture was extracted with CH₂Cl₂ (3×200 mL). The combined organic layers were dried over anhydrous Na₂SO₄, filtered and concentrated under reduced pressure. The crude residue was purified by silica gel column chromatography, eluted with PE/EA (2:1) to afford tert-butyl 4,4-difluoro-3-(2-formylpyridin-4-yl) piperidine-1-carboxylate (1.4 g, 59% yield) as a light yellow oil.

LCMS [M+H]⁺:327.

### Step 3: tert-butyl 4,4-difluoro-3-[2-(hydroxymethyl) pyridin-4-yl]piperidine-1-carboxylate

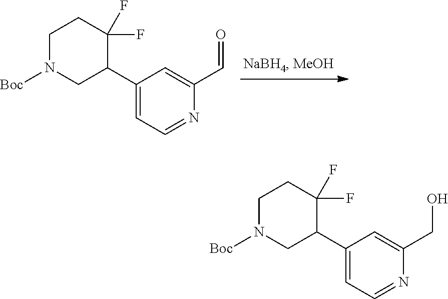 [SMILES: CC(C)(C)OC(=O)N1CCC(F)(F)C(c2ccnc(C=O)c2)C1 ; CC(C)(C)OC(=O)N1CCC(F)(F)C(c2ccnc(CO)c2)C1]

To a stirred solution of tert-butyl 4,4-difluoro-3-(2-formylpyridin-4-yl) piperidine-1-carboxylate (1.35 g, 4.1 mmol) in MeOH (20 mL) was added NaBH₄ (312 mg, 8.2 mmol) in portions at 0° C. The resulting mixture was stirred at room temperature for an additional 1 h. The reaction was quenched with water at 0° C. The resulting mixture was concentrated under reduced pressure. The crude residue was directly purified by reversed-phase flash chromatography (conditions: column, C18 silica gel; mobile phase, MeCN in water (10 mmol/L NH₄HCO₃) 30% to 70% gradient in 15 min; detector, UV 254 nm) to give tert-butyl 4,4-difluoro-3-[2-(hydroxymethyl) pyridin-4-yl]piperidine-1-carboxylate (850 mg, 62% yield) as an off-white oil.

LCMS [M+H]⁺: 329.

### Step 4: tert-butyl 4,4-difluoro-3-(2-((tosyloxy)methyl)pyridin-4-yl)piperidine-1-carboxylate

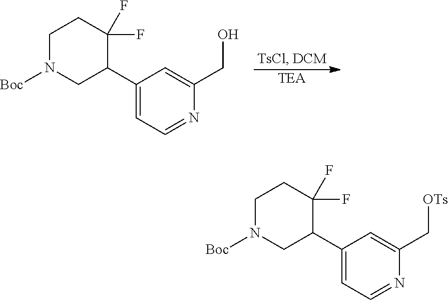

To a stirred solution of tert-butyl 4,4-difluoro-3-[2-(hydroxymethyl) pyridin-4-yl]piperidine-1-carboxylate (400 mg, 1.2 mmol) and TEA (369 mg, 3.6 mmol) in DCM (5 mL) was added TsCl (464 mg, 2.4 mmol) dropwise at 0° C. under nitrogen atmosphere. The reaction was stirred at room temperature for an additional 1 h. The resulting mixture was concentrated under reduced pressure. The crude residue was directly purified by silica gel column chromatography, eluted with PE/EA (1:5) to afford tert-butyl 4,4-difluoro-3-(2-((tosyloxy)methyl)pyridin-4-yl)piperidine-1-carboxylate (200 mg, 34% yield) as a light purple oil.

LCMS [M+H]⁺: 483.

### Step 5: 4-(1-(tert-butoxycarbonyl)-4,4-difluoropiperidin-3-yl)-2-((tosyloxy)methyl)pyridine 1-oxide

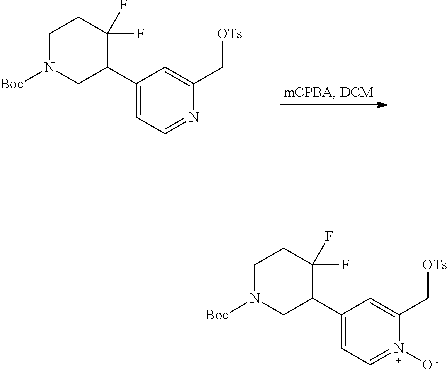

To a stirred solution of tert-butyl 4,4-difluoro-3-(2-((tosyloxy)methyl)pyridin-4-yl)piperidine-1-carboxylate (190 mg, 0.3 mmol) in DCM (4 mL) was added m-CPBA (141 mg, 0.7 mmol, 85% purity) in portions at 0° C. The resulting mixture was stirred at room temperature for an additional 1 h. The reaction was quenched with sat. Na₂S₂O₃ (aq.) at 0° C. The resulting mixture was extracted with EtOAc (3×30 mL). The combined organic layers were dried over anhydrous Na₂SO₄, filtered and concentrated under reduced pressure. The crude residue was purified by silica gel column chromatography, eluted with PE/EA (1:5) to afford 4-(1-(tert-butoxycarbonyl)-4,4-difluoropiperidin-3-yl)-2-((tosyloxy)methyl)pyridine 1-oxide (120 mg, 61% yield) as a yellow oil.

LCMS [M+H]⁺: 499.

### Step 6: 4-(1-(tert-butoxycarbonyl)-4,4-difluoropiperidin-3-yl)-2-((4,4-difluoropiperidin-1-yl)methyl)pyridine 1-oxide

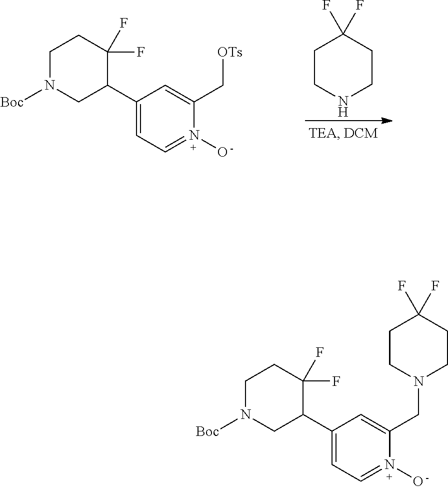

A solution of 4-(1-(tert-butoxycarbonyl)-4,4-difluoropiperidin-3-yl)-2-((tosyloxy)methyl)pyridine 1-oxide (110 mg, 0.2 mmol), TEA (111 mg, 1.1 mmol) and 4,4-difluoropiperidine (133 mg, 1.1 mmol) in DCM (3 mL) was stirred at room temperature overnight. The reaction was diluted with water. The resulting mixture was extracted with EtOAc (3×30 mL). The combined organic layers were dried over anhydrous Na₂SO₄, filtered and concentrated under reduced pressure. The crude residue was purified by silica gel column chromatography, eluted with PE/EA (1:5) to afford 4-(1-(tert-butoxycarbonyl)-4,4-difluoropiperidin-3-yl)-2-((4,4-difluoropiperidin-1-yl)methyl)pyridine 1-oxide (40 mg, 40% yield) as a light yellow oil.

LCMS [M+H]⁺: 448.

### Step 7: 2-((4,4-difluoropiperidin-1-yl)methyl)-4-(4,4-difluoropiperidin-3-yl)pyridine 1-oxide

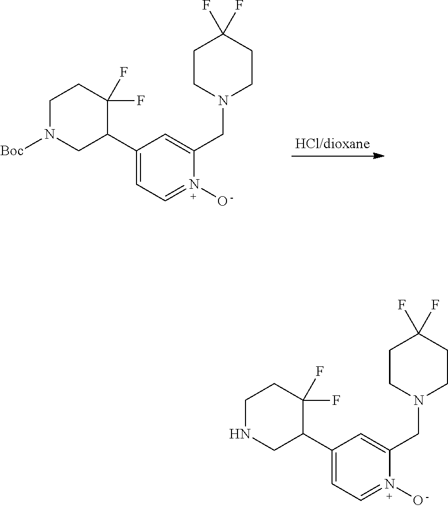

A solution of 4-(1-(tert-butoxycarbonyl)-4,4-difluoropiperidin-3-yl)-2-((4,4-difluoropiperidin-1-yl)methyl)pyridine 1-oxide (130 mg, 0.2 mmol) in HCl in 1,4-dioxane (4.0 M) (1 mL) was stirred at room temperature for 30 min. The resulting mixture was concentrated under reduced pressure. The crude residue was directly purified by reversed-phase flash chromatography (conditions: column, C18 silica gel; mobile phase, MeCN in water (0.1% formic acid), 0% to 10% gradient in 10 min; detector, UV 254 nm) to give 2-((4,4-difluoropiperidin-1-yl)methyl)-4-(4,4-difluoropiperidin-3-yl)pyridine 1-oxide (60 mg, 59% yield) as a colorless oil.

LCMS [M+H]⁺: 348.

### Step 8: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide

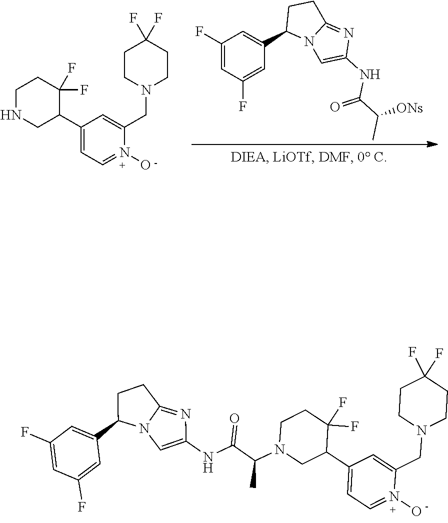

To a stirred solution of 2-((4,4-difluoropiperidin-1-yl)methyl)-4-(4,4-difluoropiperidin-3-yl)pyridine 1-oxide (50 mg, 0.1 mmol) and DIEA (55 mg, 0.4 mmol) in DMF (3 mL) was added (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (78 mg, 0.1 mmol) in portions at 0° C. under nitrogen atmosphere. The resulting mixture was stirred at 0° C. for an additional 3 days. The reaction was concentrated under reduced pressure. The crude residue was directly purified by reversed-phase flash chromatography (conditions: column, C18 silica gel; mobile phase, MeCN in water (0.1% formic acid), 30% to 60% gradient in 15 min; detector, UV 254 nm) to give 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide (30 mg, 33% yield) as a light yellow solid.

LCMS [M+H]⁺: 637.

### Step 9: 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide and 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide

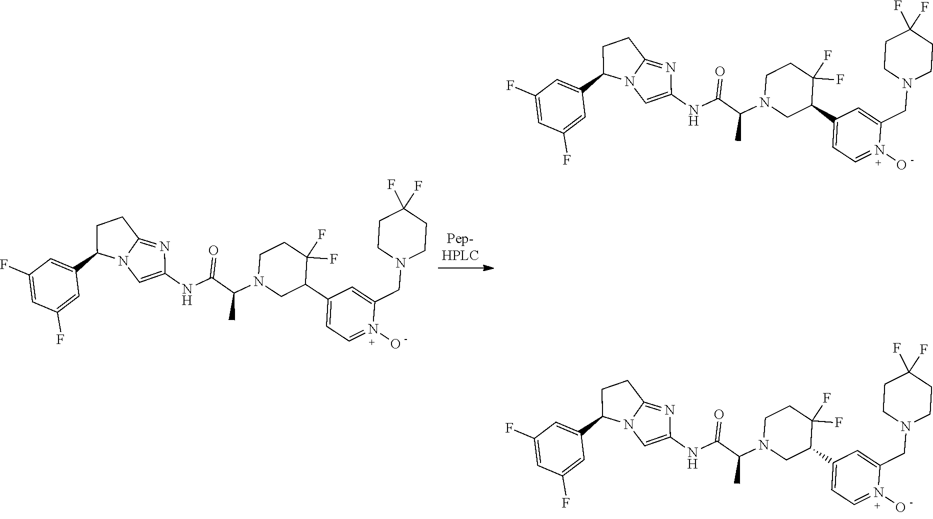

4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide (30 mg, 47 mol) was purified by Prep-HPLC (conditions: Column, Xselect CSH Prep C18, 30*150 mm 5 μm; Mobile Phase A: Water (0.1% FA), Mobile Phase B: MeOH; Flow rate: 60 mL/min; Gradient: 40% to 61% B in 12 min; Wave Length: 254 nm/220 nm; RT1 (min): 10.23 min, RT2 (min): 11.58 min) to afford first peak 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide (Compound 534) (Method AM, Peak 1, 0.95 min; 4.7 mg, 100% d.e.) as a white solid and second peak 4-((R)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-((4,4-difluoropiperidin-1-yl)methyl)pyridine 1-oxide (Compound 535) (Method AM, Peak 2, 1.07 min; 5.9 mg, 94.1% d.e.) as a white solid.

TABLE 47

| Compound# | Characterization Data |
|---|---|
| 534 | LCMS (ESI) [M + H]⁺: 637.2 |
| | ¹H NMR (400 MHz, DMSO-d₆) δ 10.16 (s, 1H), 8.21 (d, J = 6.7 Hz, 1H), 7.55 (s, 1H), 7.32- |
| | 7.26 (m, 1H), 7.25-7.17 (m, 1H), 7.01-6.89 (m, 3H), 5.41-5.34 (m, 1H), 3.72 (s, 2H), |
| | 3.61-3.47 (m, 2H), 3.03-2.91 (m, 3H), 2.90-2.86 (m, 1H), 2.83-2.74 (m, 1H), 2.73- |
| | 2.64 (m, 1H), 2.62-2.59 (m, 4H), 2.42-2.32 (m, 1H), 2.15-1.93 (m, 6H), 1.37-1.25 |
| | (m, 1H), 1.19 (d, J = 6.8 Hz, 3H). |
| 535 | LCMS (ESI) [M + H]⁺: 637.2 |
| | ¹H NMR (400 MHz, DMSO-d₆) δ 10.14 (s, 1H), 8.23 (d, J = 6.7 Hz, 1H), 7.55 (s, 1H), 7.31 |
| | (d, J = 6.7 Hz, 1H), 7.27-7.19 (m, 1H), 6.99-6.92 (m, 3H), 5.43-5.35 (m, 1H), 3.77 (bs, |
| | 2H), 3.57-3.55 (m, 2H), 2.97-2.70 (m, 6H), 2.68-2.60 (m, 2H), 2.43-2.30 (m, 2H), |
| | 2.22-1.91 (m, 7H), 1.24 (s, 1H), 1.18 (d, J = 6.8 Hz, 3H) |

### Step 1: benzyl 3-(5-(1-((tert-butylsulfinyl)amino)-2,2-difluoroethyl)-6-methoxypyridin-3-yl)-4,4-difluoropiperidine-1-carboxylate

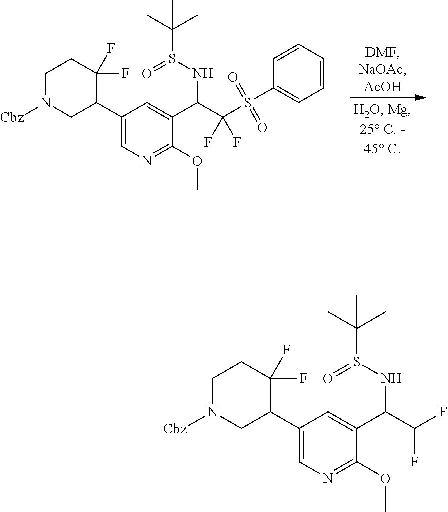 [SMILES: COc1ncc(C2CN(C(=O)OCc3ccccc3)CCC2(F)F)cc1C(NS(=O)C(C)(C)C)C(F)(F)S(=O)(=O)c1ccccc1 ; COc1ncc(C2CN(C(=O)OCc3ccccc3)CCC2(F)F)cc1C(NS(=O)C(C)(C)C)C(F)F]

To a mixture of benzyl 3-(5-(1-((tert-butylsulfinyl)amino)-2,2-difluoro-2-(phenylsulfonyl)ethyl)-6-methoxypyridin-3-yl)-4,4-difluoropiperidine-1-carboxylate (500 mg, 0.7 mmol) in DMF (10 mL) was added AcOH (437 mg, 7.3 mmol), NaOAc (598 mg, 7.3 mmol) and H₂O (1.58 g, 87.5 mmol) at room temperature under nitrogen atmosphere. The resulting mixture was stirred for 20 min. Then, magnesium (287 mg, 10.9 mmol) was added in portions over 5 min at room temperature under nitrogen atmosphere. The resulting mixture was stirred at 45° C. for an additional 1 h. The mixture was allowed to cool down to room temperature. The reaction was quenched with water (12 mL) at 0° C. The aqueous layer was extracted with EtOAc (3×20 mL). The combined organic layers were dried over anhydrous Na₂SO₄, filtered and concentrated under reduced pressure. The crude residue was purified by silica gel column chromatography, eluted with PE/EA (1:2) to afford benzyl 3-(5-(1-((tert-butylsulfinyl)amino)-2,2-difluoroethyl)-6-methoxypyridin-3-yl)-4,4-difluoropiperidine-1-carboxylate (266 mg, 66% yield) as a white solid.

LCMS (ESI) [M+H]⁺: 546.

### Step 2: 1-(5-(4,4-difluoropiperidin-3-yl)-2-methoxypyridin-3-yl)-2,2-difluoroethan-1-amine

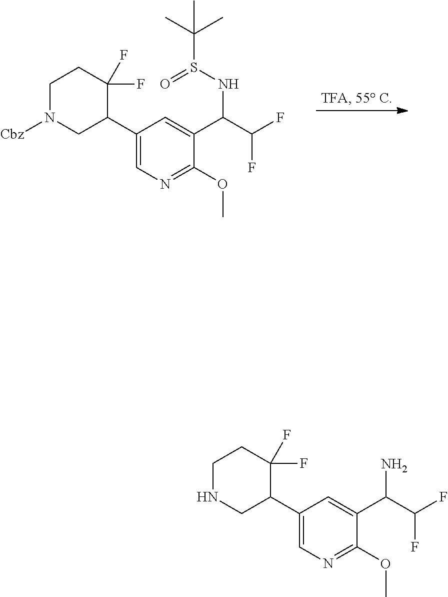 [SMILES: COc1ncc(C2CN(C(=O)OCc3ccccc3)CCC2(F)F)cc1C(NS(=O)C(C)(C)C)C(F)F ; COc1ncc(C2CNCCC2(F)F)cc1C(N)C(F)F]

A solution of benzyl 3-(5-(1-((tert-butylsulfinyl)amino)-2,2-difluoroethyl)-6-methoxypyridin-3-yl)-4,4-difluoropiperidine-1-carboxylate (250 mg, 0.5 mmol) in TFA (6 mL) was stirred at 55° C. for 6 h under nitrogen atmosphere. The mixture was allowed to cool down to room temperature. The resulting mixture was concentrated under reduced pressure. The crude residue was directly purified by reversed-phase flash chromatography (conditions: column, C18 silica gel; mobile phase, MeCN in water (10 mmol/L NH₄HCO₃), 0% to 100% gradient in 20 min; detector, UV 254 nm) to give 1-(5-(4,4-difluoropiperidin-3-yl)-2-methoxypyridin-3-yl)-2,2-difluoroethan-1-amine (108 mg, 76% yield) as a yellow solid.

LCMS (ESI) [M+H]⁺: 308.

### Step 3: (2S)-2-(3-(5-(1-amino-2,2-difluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

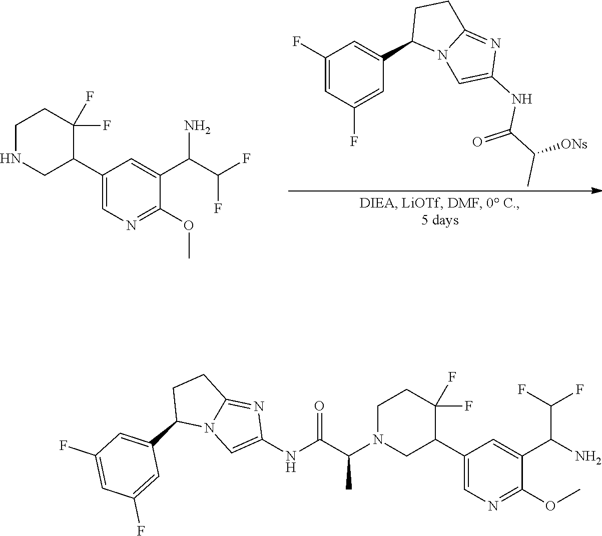

To a mixture of 1-(5-(4,4-difluoropiperidin-3-yl)-2-methoxypyridin-3-yl)-2,2-difluoroethan-1-amine (68 mg, 0.2 mmol) in DMF (5 mL) was added DIEA (85 mg, 0.7 mmol), lithium trifluoromethanesulfonate (103 mg, 0.7 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (108 mg, 0.2 mmol) dropwise at 0° C. under nitrogen atmosphere. The resulting mixture was stirred at 0° C. for 5 days under nitrogen atmosphere. The reaction mixture was directly purified by reversed-phase flash chromatography (conditions: column, C18 silica gel; mobile phase, ACN in water (0.1% NH₄HCO₃), 5% to 100% gradient in 25 min; detector, UV 254 nm) to give (2S)-2-(3-(5-(1-amino-2,2-difluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (42 mg, 31% yield) as a yellow solid.

LCMS (ESI) [M+H]⁺: 597.

### Step 4: (2S)-2-(3-(5-(1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

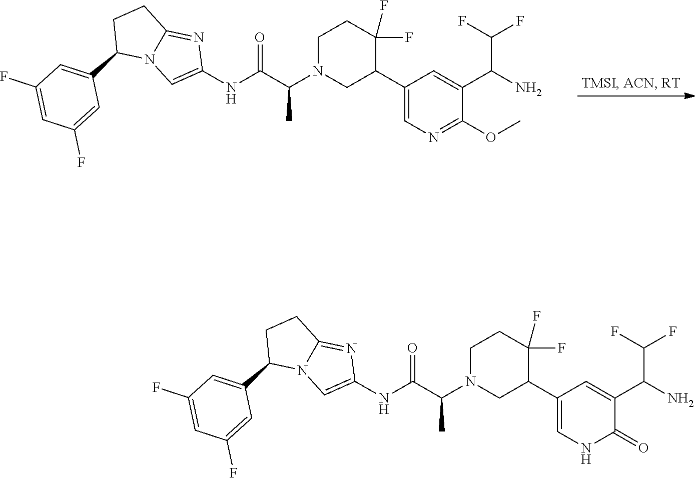 [SMILES: COc1ncc(C2CN([C@@H](C)C(=O)Nc3cn4c(n3)CC[C@@H]4c3cc(F)cc(F)c3)CCC2(F)F)cc1C(N)C(F)F ; C[C@@H](C(=O)Nc1cn2c(n1)CC[C@@H]2c1cc(F)cc(F)c1)N1CCC(F)(F)C(c2c[nH]c(=O)c(C(N)C(F)F)c2)C1]

To a mixture of (2S)-2-(3-(5-(1-amino-2,2-difluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (37 mg, 60.0 μmol) in ACN (3 mL) was added iodotrimethylsilane (62 mg, 0.3 mmol) dropwise at 0° C. under nitrogen atmosphere. The reaction was stirred at room temperature for 4 h. The resulting mixture was concentrated under reduced pressure. The crude residue was directly purified by reversed-phase flash chromatography (conditions: column, C18 silica gel; mobile phase, MeCN in water (10 mmol/L NH₄HCO₃), 5% to 100% gradient in 25 min; detector, UV 254 nm) to give (2S)-2-(3-(5-(1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (29 mg, 80% yield) as a white solid.

LCMS (ESI) [M+H]⁺: 583.

### Step 4: (S)-2-((S)-3-(5-((S)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide and (S)-2-((R)-3-(5-((S)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide and (S)-2-((S)-3-(5-((R)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide and (S)-2-((R)-3-(5-((R)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

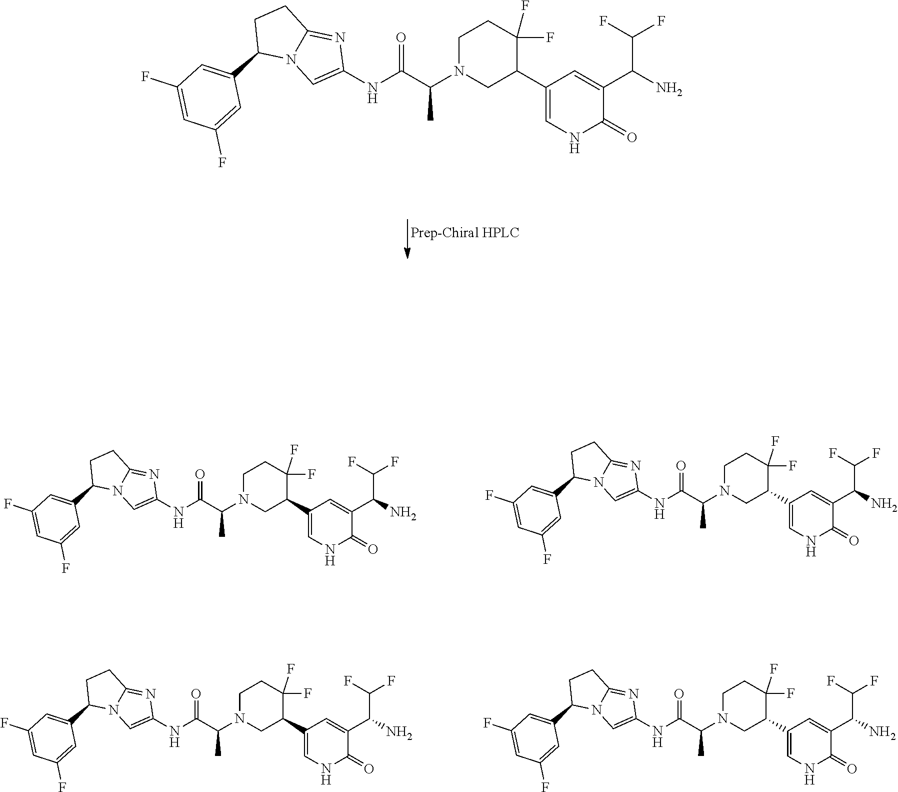 [SMILES: C[C@@H](C(=O)Nc1cn2c(n1)CC[C@@H]2c1cc(F)cc(F)c1)N1CCC(F)(F)C(c2c[nH]c(=O)c(C(N)C(F)F)c2)C1 ; C[C@@H](C(=O)Nc1cn2c(n1)CC[C@@H]2c1cc(F)cc(F)c1)N1CCC(F)(F)[C@@H](c2c[nH]c(=O)c([C@H](N)C(F)F)c2)C1 ; C[C@@H](C(=O)Nc1cn2c(n1)CC[C@@H]2c1cc(F)cc(F)c1)N1CCC(F)(F)[C@H](c2c[nH]c(=O)c([C@H](N)C(F)F)c2)C1 ; C[C@@H](C(=O)Nc1cn2c(n1)CC[C@@H]2c1cc(F)cc(F)c1)N1CCC(F)(F)[C@@H](c2c[nH]c(=O)c([C@@H](N)C(F)F)c2)C1 ; C[C@@H](C(=O)Nc1cn2c(n1)CC[C@@H]2c1cc(F)cc(F)c1)N1CCC(F)(F)[C@H](c2c[nH]c(=O)c([C@@H](N)C(F)F)c2)C1]

(2S)-2-(3-(5-(1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (29 mg) was separated by Prep-Chiral HPLC with the following conditions (Column: (S, S)-WHELK-O1, 2*25 cm, 5 μm; Mobile Phase A: Hex (10 mM NH₃-MeOH), Mobile Phase B: EtOH; Flow rate: 20 mL/min; Gradient: isocratic 50% B; Wave Length: 234/208 nm; RT1 (min): 11.79; RT2 (min): 14.80), each as a mixture of diastereomers. The first portion was separated by Prep-Chiral HPLC with the following conditions (Column: Lux 5 μm Cellulose-4 3*25 cm, 5 μm; Mobile Phase A: Hex (10 mM NH₃-MeOH), Mobile Phase B: EtOH; Flow rate: 40 mL/min; Gradient: isocratic 30% B; Wave Length: 308/234 nm; RT1 (min): 17.54; RT2 (min): 22.72) to afford first peak (S)-2-((S)-3-(5-((S)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 536) (Method AN, Peak 1, 2.36 min; 3.2 mg, 100% d.e.) as a white solid and second peak (S)-2-((R)-3-(5-((S)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 537) (Method AN, Peak 2, 3.17 min; 2.6 mg, 100% d.e.) as a white solid.

The second portion was separated by Prep-Chiral HPLC with the following conditions (Column: Lux 5 μm Cellulose-2, 30*250 mm, 5.0 μm; Mobile Phase A: Hex (10 mM NH₃-MeOH), Mobile Phase B: EtOH; Flow rate: 40 mL/min; Gradient: isocratic 30% B; Wave Length: 334/306 nm; RT1 (min): 17.91; RT2 (min): 23.39) to afford first peak (S)-2-((S)-3-(5-((R)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 538) (Method AO, Peak 1, 2.27 min; 3.2 mg, 100% d.e.) as a white solid and second peak (S)-2-((R)-3-(5-((R)-1-amino-2,2-difluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 539) (Method AO, Peak 2, 3.09 min; 1.3 mg, 96% d.e.) as a white solid.

TABLE 48

| Compound# | Characterization Data |
|---|---|
| 536 | LCMS (ESI) [M + H]⁺: 583.1 |
| | ¹H NMR (400 MHz, DMSO-d₆) δ 11.74 (bs, 1H), 10.10 (s, 1H), 7.55 (d, J = 2.4 Hz, 1H), 7.31 |
| | (d, J = 2.4 Hz, 1H), 7.22-7.18 (m, 1H), 6.99-6.92 (m, 3H), 6.18-5.91 (m, 1H), 5.41-5.34 |
| | (m, 1H), 4.09-3.98 (m, 1H), 3.52-3.41 (m, 1H), 3.24-3.12 (m, 1H), 3.01-2.75 (m, 6H), |
| | 2.61-2.59 (m, 2H), 2.41-2.31 (m, 1H), 2.15-1.92 (m, 3H), 1.17 (d, J = 6.8 Hz, 3H). |
| 537 | LCMS (ESI) [M + H]⁺: 583.1 |
| | ¹H NMR (400 MHz, DMSO-d₆) δ 11.75 (bs, 1H), 10.07 (s, 1H), 7.54 (d, J = 2.4 Hz, 1H), |
| | 7.27-7.13 (m, 2H), 6.99-6.92 (m, 3H), 6.21-5.91 (m, 1H), 5.42-5.34 (m, 1H), 4.11-3.99 (m, |
| | 1H), 3.53-3.45 (m, 1H), 3.25-3.14 (m, 1H), 3.06-2.71 (m, 7H), 2.47-2.35 (m, 2H), |
| | 2.15-1.92 (m, 3H), 1.17 (d, J = 6.8 Hz, 3H). |
| 538 | LCMS (ESI) [M + H]⁺: 583.1 |
| | ¹H NMR (400 MHz, DMSO-d₆) δ 11.75 (bs, 1H), 10.07 (s, 1H), 7.54 (s, 1H), 7.31 (s, 1H), |
| | 7.27-7.17 (m, 1H), 6.99-6.92 (m, 3H), 6.19-5.92 (m, 1H), 5.39-5.32 (m, 1H), 4.12- |
| | 4.01 (m, 1H), 3.53-3.44 (m, 1H), 3.25-3.13 (m, 1H), 3.06-2.72 (m, 6H), 2.67-2.52 (m, |
| | 2H), 2.41-2.32 (m, 1H), 2.19-1.92 (m, 3H), 1.17 (d, J = 6.8 Hz, 3H). |
| 539 | LCMS (ESI) [M + H]⁺: 583.2 |
| | ¹H NMR (400 MHz, DMSO-d₆) δ 11.76 (bs, 1H), 10.07 (s, 1H), 7.54 (s, 1H), 7.31-7.17 (m, |
| | 2H), 6.99-6.92 (m, 3H), 6.21-5.93 (m, 1H), 5.39-5.32 (m, 1H), 4.12-3.97 (m, 1H), |
| | 3.54-3.45 (m, 1H), 3.25-3.13 (m, 1H), 3.06-2.72 (m, 7H), 2.41-2.32 (m, 1H), |
| | 2.19-1.92 (m, 4H), 1.17 (d, J = 6.8 Hz, 3H). |

### Example 74: (2S)-2-(3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

### Step 1: benzyl (E)-3-(5-(((tert-butylsulfinyl)imino)methyl)-6-methoxypyridin-3-yl)-4,4-difluoropiperidine-1-carboxylate

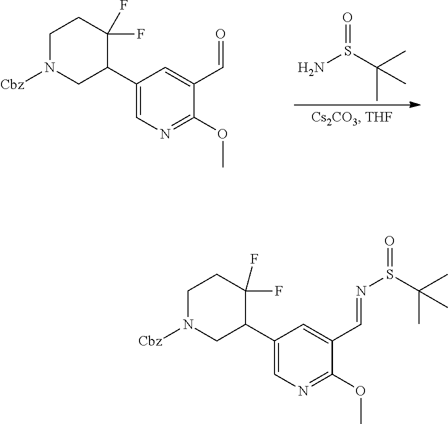

To a mixture of benzyl 4,4-difluoro-3-(5-formyl-6-methoxypyridin-3-yl)piperidine-1-carboxylate (2.00 g, 5.1 mmol) in THF (30 mL) were added tert-butanesulfinamide (620 mg, 5.1 mmol) and Cs₂CO₃ (5.01 g, 15.3 mmol). The reaction was stirred at room temperature overnight under nitrogen atmosphere. The resulting mixture was concentrated under reduced pressure. The crude residue was purified by silica gel column chromatography, eluted with CH₂Cl₂/EA (5:1) to afford benzyl (E)-3-(5-(((tert-butylsulfinyl)imino)methyl)-6-methoxypyridin-3-yl)-4,4-difluoropiperidine-1-carboxylate (1.10 g, 43% yield) as a yellow solid. LCMS (ESI) [M+H]⁺: 494.

### Step 2: benzyl 3-(5-(1-((tert-butylsulfinyl)amino)-2,2,2-trifluoroethyl)-6-methoxypyridin-3-yl)-4,4-difluoropiperidine-1-carboxylate

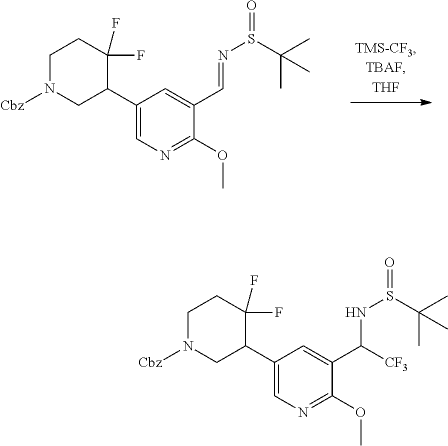 [SMILES: COc1ncc(C2CN(C(=O)OCc3ccccc3)CCC2(F)F)cc1/C=N/S(=O)C(C)(C)C ; COc1ncc(C2CN(C(=O)OCc3ccccc3)CCC2(F)F)cc1C(NS(=O)C(C)(C)C)C(F)(F)F]

A solution of benzyl (E)-3-(5-(((tert-butylsulfinyl)imino)methyl)-6-methoxypyridin-3-yl)-4,4-difluoropiperidine-1-carboxylate (1.10 g, 2.2 mmol), tetra-n-butylammonium fluoride in THF (116 mg, 0.5 mmol, 1 M in THF) and trimethyl(trifluoromethyl)silane (950 mg, 6.7 mmol) in THF (15 mL) was stirred at room temperature for 2 h under nitrogen atmosphere. The resulting mixture was concentrated under reduced pressure. The crude residue was purified by reversed-phase flash chromatography (conditions: column, C18 silica gel; mobile phase, MeCN in water (10 mmol/L NH₄HCO₃), 0% to 100% gradient in 20 min; detector, UV 254 nm) to afford benzyl 3-(5-(1-((tert-butylsulfinyl)amino)-2,2,2-trifluoroethyl)-6-methoxypyridin-3-yl)-4,4-difluoropiperidine-1-carboxylate (780 mg, 62% yield) as a yellow solid. LCMS (ESI) [M+H]⁺: 564.

### Step 3: 1-(5-(4,4-difluoropiperidin-3-yl)-2-methoxypyridin-3-yl)-2,2,2-trifluoroethan-1-amine

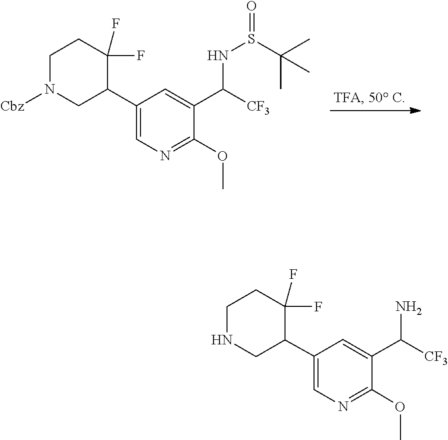 [SMILES: COc1ncc(C2CN(C(=O)OCc3ccccc3)CCC2(F)F)cc1C(NS(=O)C(C)(C)C)C(F)(F)F ; COc1ncc(C2CNCCC2(F)F)cc1C(N)C(F)(F)F]

A solution of benzyl 3-(5-(1-((tert-butylsulfinyl)amino)-2,2,2-trifluoroethyl)-6-methoxypyridin-3-yl)-4,4-difluoropiperidine-1-carboxylate (780 mg, 1.4 mmol) in TFA (10 mL) was stirred at 55° C. for 4 h under nitrogen atmosphere. The mixture was allowed to cool down to room temperature. The resulting mixture was concentrated under reduced pressure. The crude residue was purified by reversed-phase flash chromatography (conditions: column, C18 silica gel; mobile phase, MeCN in water (10 mmol/L NH₄HCO₃), 0% to 100% gradient in 20 min; detector, UV 254 nm and UV 220 nm) to afford 1-(5-(4,4-difluoropiperidin-3-yl)-2-methoxypyridin-3-yl)-2,2,2-trifluoroethan-1-amine (332 mg, 73% yield) as a yellow oil. LCMS (ESI) [M+H]⁺: 326.

### Step 4: (2S)-2-(3-(5-(1-amino-2,2,2-trifluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

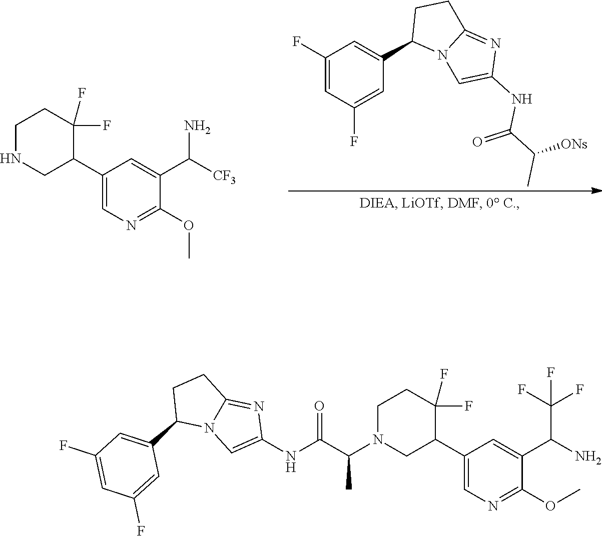

To a mixture of 1-(5-(4,4-difluoropiperidin-3-yl)-2-methoxypyridin-3-yl)-2,2,2-trifluoroethan-1-amine (120 mg, 0.4 mmol) in DMF (8 mL) were added DIEA (143 mg, 1.1 mmol), lithium trifluoromethanesulfonate (172 mg, 1.1 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (181 mg, 0.4 mmol) dropwise at 0° C. under nitrogen atmosphere. The resulting mixture was stirred at 0° C. for 5 days. The reaction mixture was directly purified by reversed-phase flash chromatography (conditions: column, C18 silica gel; mobile phase, MeCN in water (10 mmol/L NH₄HCO₃), 0% to 100% gradient in 20 min; detector, UV 254 nm) to afford (2S)-2-(3-(5-(1-amino-2,2,2-trifluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (118 mg, 52% yield) as a white solid. LCMS (ESI) [M+H]⁺: 615.

### Step 5: (2S)-2-(3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

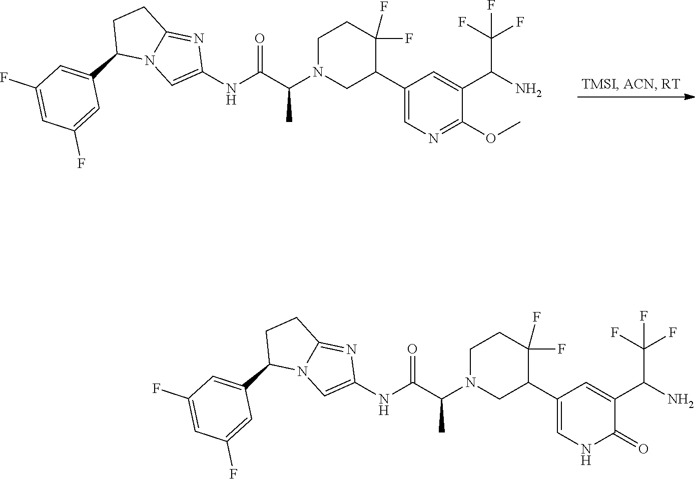 [SMILES: COc1ncc(C2CN([C@@H](C)C(=O)Nc3cn4c(n3)CC[C@@H]4c3cc(F)cc(F)c3)CCC2(F)F)cc1C(N)C(F)(F)F ; C[C@@H](C(=O)Nc1cn2c(n1)CC[C@@H]2c1cc(F)cc(F)c1)N1CCC(F)(F)C(c2c[nH]c(=O)c(C(N)C(F)(F)F)c2)C1]

To a mixture of (2S)-2-(3-(5-(1-amino-2,2,2-trifluoroethyl)-6-methoxypyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (110 mg, 0.2 mmol) in ACN (5 mL) was added iodotrimethylsilane (179 mg, 0.9 mmol) dropwise at 0° C. under nitrogen atmosphere. The resulting mixture was stirred at room temperature for 1 h. The resulting mixture was concentrated under reduced pressure. The crude residue was purified by reversed-phase flash chromatography (conditions: column, C18 silica gel; mobile phase, ACN in water (0.1% formic acid), 0% to 100% gradient in 20 min; detector, UV 254 nm) to afford (2S)-2-(3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (40 mg, 37% yield) as a white solid. LCMS (ESI) [M+H]⁺: 601.

### Step 6: (2S)-2-((3S)-3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide and (2S)-2-((3R)-3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide

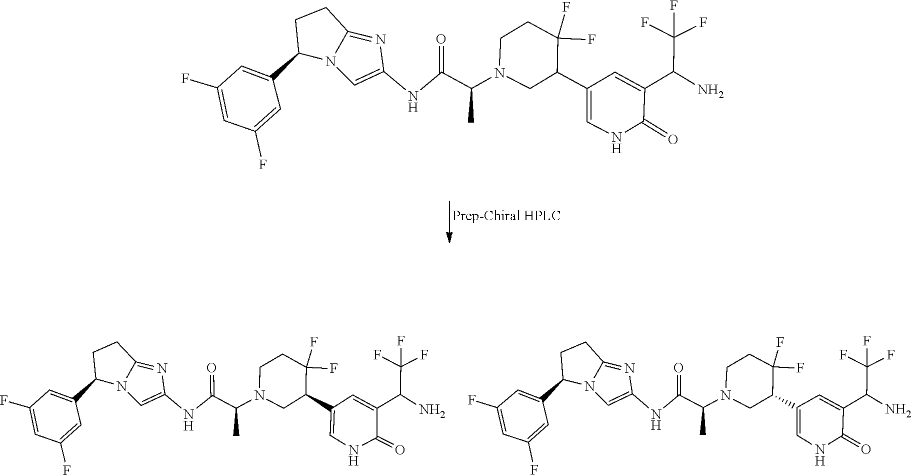 [SMILES: C[C@@H](C(=O)Nc1cn2c(n1)CC[C@@H]2c1cc(F)cc(F)c1)N1CCC(F)(F)C(c2c[nH]c(=O)c(C(N)C(F)(F)F)c2)C1 ; C[C@@H](C(=O)Nc1cn2c(n1)CC[C@@H]2c1cc(F)cc(F)c1)N1CCC(F)(F)[C@@H](c2c[nH]c(=O)c(C(N)C(F)(F)F)c2)C1 ; C[C@@H](C(=O)Nc1cn2c(n1)CC[C@@H]2c1cc(F)cc(F)c1)N1CCC(F)(F)[C@H](c2c[nH]c(=O)c(C(N)C(F)(F)F)c2)C1]

(2S)-2-(3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (40 mg) was separated by Prep-HPLC with the following conditions (Column: Xselect CSH Prep C18, 30*150 mm 5 μm; Mobile Phase A: Water (0.1% FA), Mobile Phase B: MeOH; Flow rate: 60 mL/min; Gradient (B %): 35% to 55% in 12 min; Wave Length: 254 nm/220 nm; RT (min): 10.43/12.60 min) to afford first peak (2S)-2-((3S)-3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 540) (14.3 mg, 36% yield, 95.3% purity, as a mixture of diastereomers) as a white solid and second peak (2S)-2-((3R)-3-(5-(1-amino-2,2,2-trifluoroethyl)-6-oxo-1,6-dihydropyridin-3-yl)-4,4-difluoropiperidin-1-yl)-N—((R)-5-(3,5-difluorophenyl)-6,7-dihydro-5H-pyrrolo[1,2-a]imidazol-2-yl)propanamide (Compound 541) (14.3 mg, 32% yield, 98.4% purity, as a mixture of diastereomers) as a white solid.

TABLE 49

| Compound# | Characterization Data |
|---|---|
| 540 | LCMS (ESI) [M + H]⁺: 601.2 |
| | ¹H NMR (400 MHz, DMSO-d₆) δ 11.78 (bs, 1H), 10.11 (d, J = 8.8 Hz, 1H), 7.68-7.65 (m, |
| | 1H), 7.34 (s, 1H), 7.26-7.17 (m, 1H), 6.98-6.90 (m, 3H), 5.41-5.34 (m, 1H), 4.59-4.53 |
| | (m, 1H), 3.51-3.42 (m, 1H), 3.22-3.10 (m, 1H), 2.99-2.76 (m, 5H), 2.64-2.55 (m, 2H), |
| | 2.43-2.38 (m, 1H), 2.09-1.92 (m, 2H), 1.17 (d, J = 6.8 Hz, 3H). |
| 541 | LCMS (ESI) [M + H]⁺: 601.2 |
| | ¹H NMR (400 MHz, DMSO-d₆) δ 11.81 (bs, 1H), 10.07 (d, J = 11.7 Hz, 1H), 7.68 (s, 1H), |
| | 7.32 (s, 1H), 7.27-7.18 (m, 1H), 7.00-6.91 (m, 3H), 5.45-5.31 (m, 1H), 4.65-4.55 (m, |
| | 1H), 3.54-3.51 (m, 1H), 3.23-3.14 (m, 1H), 3.02-2.69 (m, 7H), 2.47-2.31 (m, 1H), |
| | 2.12-1.92 (m, 2H), 1.17 (d, J = 6.9 Hz, 3H). |

### Example 75: (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-oxo-1,6-dihydropyridin-3-yl)-4,4-difluoropiperidin-1-yl)propanamide

### Step 1: benzyl 3-(5-((4,4-difluoropiperidin-1-yl)methyl)-6-methoxypyridin-3-yl)-4,4-difluoropiperidine-1-carboxylate

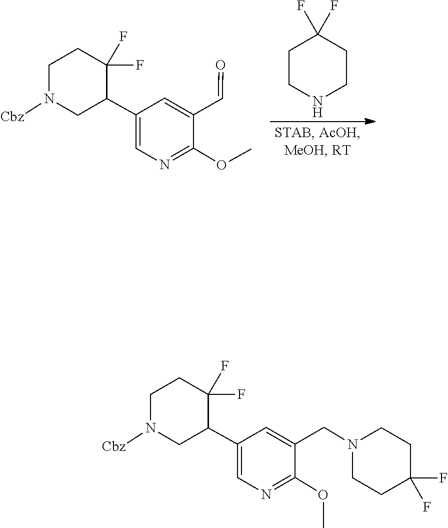 [SMILES: COc1ncc(C2CN(C(=O)OCc3ccccc3)CCC2(F)F)cc1C=O ; FC1(F)CCNCC1 ; COc1ncc(C2CN(C(=O)OCc3ccccc3)CCC2(F)F)cc1CN1CCC(F)(F)CC1]

To a mixture of benzyl 4,4-difluoro-3-(5-formyl-6-methoxypyridin-3-yl)piperidine-1-carboxylate (400 mg, 1.0 mmol) in MeOH (10 mL) were added 4,4-difluoropiperidine (149 mg, 1.2 mmol) and AcOH (6 mg, 0.1 mmol) dropwise at 0° C. under nitrogen atmosphere. The resulting mixture was stirred at room temperature for 1 h. To the above mixture was added STAB (326 mg, 1.5 mmol) in portions over 5 min at 0° C. The resulting mixture was stirred at room temperature for an additional 2 h. The reaction was quenched with water (12 mL) at 0° C. The aqueous layer was extracted with EtOAc (3×20 mL). The combined organic layers were dried over anhydrous Na₂SO₄, filtered and concentrated under reduced pressure. The crude residue was purified by silica gel column chromatography, eluted with PE/EA (1:1) to afford benzyl 3-(5-((4,4-difluoropiperidin-1-yl)methyl)-6-methoxypyridin-3-yl)-4,4-difluoropiperidine-1-carboxylate (420 mg, 83% yield) as a colorless oil. LCMS (ESI) [M+H]⁺: 496.

### Step 2: 3-((4,4-difluoropiperidin-1-yl)methyl)-5-(4,4-difluoropiperidin-3-yl)-2-methoxypyridine

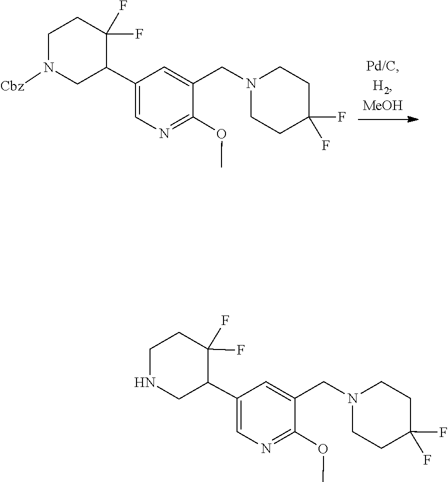 [SMILES: COc1ncc(C2CN(C(=O)OCc3ccccc3)CCC2(F)F)cc1CN1CCC(F)(F)CC1 ; COc1ncc(C2CNCCC2(F)F)cc1CN1CCC(F)(F)CC1]

A solution of benzyl 3-(5-((4,4-difluoropiperidin-1-yl)methyl)-6-methoxypyridin-3-yl)-4,4-difluoropiperidine-1-carboxylate (420 mg, 0.9 mmol) and 10% Pd/C (50 mg, anhydrous) in MeOH (8 mL) was stirred at room temperature for 2 h under hydrogen atmosphere. The resulting mixture was filtered through celite. The filter cake was washed with MeOH. The filtrate was concentrated under reduced pressure. The crude residue was purified by reversed-phase flash chromatography (conditions: column, C18 silica gel; mobile phase, ACN in water (0.1% NH₄HCO₃), 0% to 100% gradient in 20 min; detector, UV 254 nm) to afford 3-((4,4-difluoropiperidin-1-yl)methyl)-5-(4,4-difluoropiperidin-3-yl)-2-methoxypyridine (220 mg, 72% yield) as a colorless oil. LCMS (ESI) [M+H]⁺: 362.

### Step 3: (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-methoxypyridin-3-yl)-4,4-difluoropiperidin-1-yl)propanamide

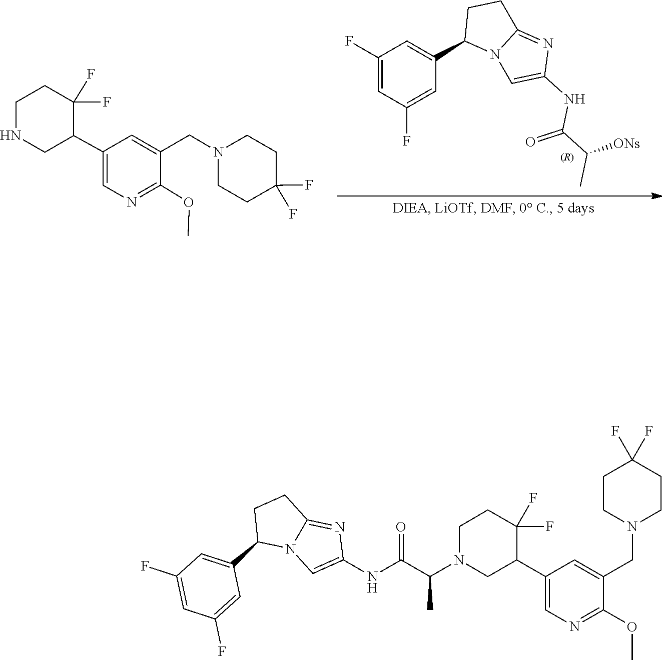

A solution of 3-((4,4-difluoropiperidin-1-yl)methyl)-5-(4,4-difluoropiperidin-3-yl)-2-methoxypyridine (220 mg, 0.6 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (359 mg, 0.7 mmol), DIEA (196 mg, 1.5 mmol) and lithium trifluoromethanesulfonate (246 mg, 1.6 mmol) in DMF (8 mL) was stirred at 0° C. for 5 days under nitrogen atmosphere. The resulting mixture was directly purified by reversed-phase flash chromatography (conditions: column, C18 silica gel; mobile phase, ACN in water (0.1% NH₄HCO₃), 0% to 100% gradient in 20 min; detector, UV 254 nm) to afford (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-methoxypyridin-3-yl)-4,4-difluoropiperidin-1-yl)propanamide (250 mg, 63% yield) as a light yellow solid. LCMS (ESI) [M+H]⁺: 651.

### Step 4: (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-oxo-1,6-dihydropyridin-3-yl)-4,4-difluoropiperidin-1-yl)propanamide

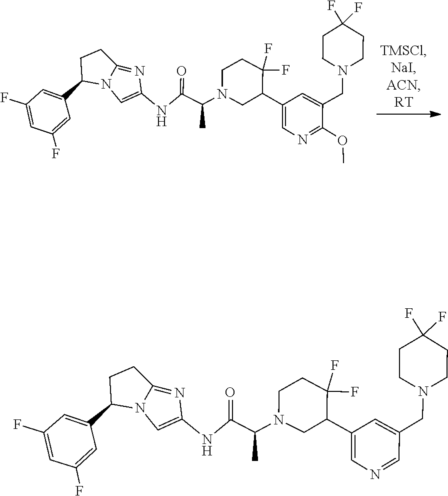 [SMILES: COc1ncc(C2CN([C@@H](C)C(=O)Nc3cn4c(n3)CC[C@@H]4c3cc(F)cc(F)c3)CCC2(F)F)cc1CN1CCC(F)(F)CC1 ; C[C@@H](C(=O)Nc1cn2c(n1)CC[C@@H]2c1cc(F)cc(F)c1)N1CCC(F)(F)C(c2cncc(CN3CCC(F)(F)CC3)c2)C1]

To a mixture of (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-methoxypyridin-3-yl)-4,4-difluoropiperidin-1-yl)propanamide (200 mg, 0.3 mmol) in ACN (5 mL) were added NaI (230 mg, 1.5 mmol) and TMSCl (166 mg, 1.5 mmol) dropwise over 1 min at 0° C. The reaction was stirred at room temperature for an additional 3 h. The resulting mixture was concentrated under reduced pressure. The crude residue was purified by reversed-phase flash chromatography (conditions: column, C18 silica gel; mobile phase, ACN in water (0.1% formic acid), 0% to 100% gradient in 22 min; detector, UV 254 nm) to afford (2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-oxo-1,6-dihydropyridin-3-yl)-4,4-difluoropiperidin-1-yl)propanamide (120 mg, 61% yield) as a colorless oil. LCMS (ESI) [M+H]⁺: 637.

### Step 5: (S)—N—((R)-5-(3,5-difluorophenyl)-6,7-dihydro-5H-pyrrolo[1,2-a]imidazol-2-yl)-2-((S)-3-(5-((4,4-difluoropiperidin-1-yl)methyl)-6-oxo-1,6-dihydropyridin-3-yl)-4,4-difluoropiperidin-1-yl)propanamide and (S)—N—((R)-5-(3,5-difluorophenyl)-6,7-dihydro-5H-pyrrolo[1,2-a]imidazol-2-yl)-2-((R)-3-(5-((4,4-difluoropiperidin-1-yl)methyl)-6-oxo-1,6-dihydropyridin-3-yl)-4,4-difluoropiperidin-1-yl)propanamide

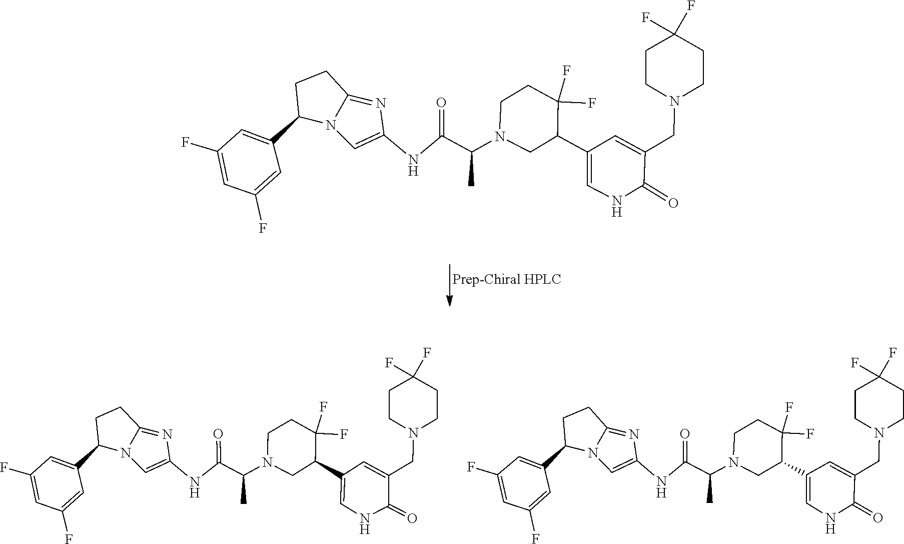 [SMILES: C[C@@H](C(=O)Nc1cn2c(n1)CC[C@@H]2c1cc(F)cc(F)c1)N1CCC(F)(F)C(c2c[nH]c(=O)c(CN3CCC(F)(F)CC3)c2)C1 ; C[C@@H](C(=O)Nc1cn2c(n1)CC[C@@H]2c1cc(F)cc(F)c1)N1CCC(F)(F)[C@@H](c2c[nH]c(=O)c(CN3CCC(F)(F)CC3)c2)C1 ; C[C@@H](C(=O)Nc1cn2c(n1)CC[C@@H]2c1cc(F)cc(F)c1)N1CCC(F)(F)[C@H](c2c[nH]c(=O)c(CN3CCC(F)(F)CC3)c2)C1]

(2S)—N—((R)-5-(3,5-difluorophenyl)-6,7-dihydro-5H-pyrrolo[1,2-a]imidazol-2-yl)-2-(3-(5-((4,4-difluoropiperidin-1-yl)methyl)-6-oxo-1,6-dihydropyridin-3-yl)-4,4-difluoropiperidin-1-yl)propanamide (120 mg) was separated by Prep-HPLC with the following conditions (Column: Xselect CSH Prep C18, 30*150 mm 5 μm; Mobile Phase A: Water (0.1% FA), Mobile Phase B: MeOH; Flow rate: 60 mL/min; Gradient (B %): 21% to 41% in 12 min; Wave Length: 254 nm/220 nm; RT1 (min): 10.92; RT2 (min): 12.85) to afford first peak (S)—N—((R)-5-(3,5-difluorophenyl)-6,7-dihydro-5H-pyrrolo[1,2-a]imidazol-2-yl)-2-((S)-3-(5-((4,4-difluoropiperidin-1-yl)methyl)-6-oxo-1,6-dihydropyridin-3-yl)-4,4-difluoropiperidin-1-yl)propanamide (Compound 542) (Method AP, Peak 1, 1.07 min; 45.8 mg, 100% d.e.) as a white solid and second peak (S)—N—((R)-5-(3,5-difluorophenyl)-6,7-dihydro-5H-pyrrolo[1,2-a]imidazol-2-yl)-2-((R)-3-(5-((4,4-difluoropiperidin-1-yl)methyl)-6-oxo-1,6-dihydropyridin-3-yl)-4,4-difluoropiperidin-1-yl)propanamide (Compound 543) (Method AP, Peak 2, 1.61 min; 31.4 mg, 96.3% d.e.) as a white solid.

TABLE 50

| Compound# | Characterization Data |
|---|---|
| 542 | LCMS (ESI) [M + H]⁺: 637.2 |
| | ¹H NMR (400 MHz, DMSO-d₆) δ 11.53 (bs, 1H), 10.08 (s, 1H), 7.40 (s, 1H), 7.25-7.15 (m, |
| | 2H), 6.99-6.89 (m, 3H), 5.41-5.32 (m, 1H), 3.51-3.42 (m, 1H), 3.33 (s, 2H), 3.23-3.12 |
| | (m, 1H), 3.00-2.85 (m, 4H), 2.82-2.73 (m, 1H), 2.70-2.54 (m, 2H), 2.49-2.47 (m, 4H), |
| | 2.41-2.26 (m, 1H), 2.12-1.83 (m, 6H), 1.17 (d, J = 6.9 Hz, 3H). |
| 543 | LCMS (ESI) [M + H]⁺: 637. 30 |
| | ¹H NMR (400 MHz, DMSO-d₆) δ 11.54 (bs, 1H), 10.04 (s, 1H), 7.42 (s, 1H), 7.28-7.17 (m, |
| | 2H), 7.03-6.89 (m, 3H), 5.43-5.31 (m, 1H), 3.51-3.47 (m, 1H), 3.35 (s, 2H), 3.21-3.12 |
| | (m, 1H), 3.03-2.85 (m, 4H), 2.83-2.66 (m, 2H), 2.53-2.50 (m, 5H), 2.38-2.30 (m, 1H), |
| | 2.10-1.86 (m, 6H), 1.16 (d, J = 6.9 Hz, 3H). |

### Example 76: (2S)-2-(4,4-difluoro-3-(6-(hydroxymethyl)-5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide

### Step 1: 5-bromo-2-methoxy-3-[(methoxymethoxy)methyl]pyrazine

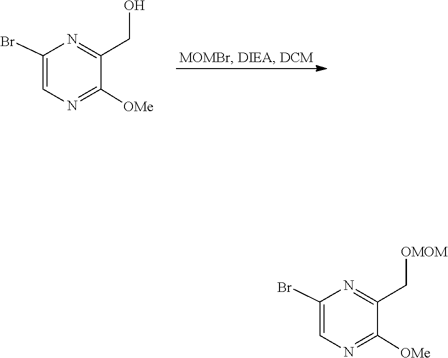

To a stirred solution of (6-bromo-3-methoxypyrazin-2-yl)methanol (4 g, 18.3 mmol) and DIEA (7.08 g, 54.8 mmol) in DCM (40 mL) was added bromo(methoxy)methane (2.97 g, 23.7 mmol) dropwise at 0° C. under nitrogen atmosphere. The mixture was stirred at 0° C. for 1 h. The mixture was concentrated under reduced pressure. The crude residue was purified by flash chromatography on silica gel eluting with EA/PE (2:3) to afford 5-bromo-2-methoxy-3-[(methoxymethoxy)methyl]pyrazine (4.2 g, 87% yield) as an off-white solid. LCMS [M+H]⁺: 264.

### Step 2: Benzyl 3-(5-methoxy-6-((methoxymethoxy)methyl)pyrazin-2-yl)-4-oxopiperidine-1-carboxylate

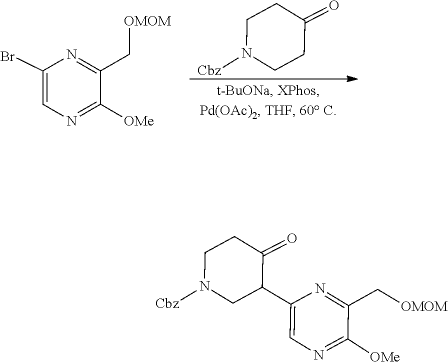

A solution of 5-bromo-2-methoxy-3-[(methoxymethoxy)methyl]pyrazine (4.2 g, 16.0 mmol), t-BuONa (4.6 g, 47.9 mmol), XPhos (1.5 g, 3.2 mmol), Pd(OAc)₂ (720 mg, 3.2 mmol) and benzyl 4-oxopiperidine-1-carboxylate (5.60 g, 23.9 mmol) in THF (100 mL) was stirred at 60° C. for 2 h under nitrogen atmosphere. The mixture was allowed to cool down to room temperature. The resulting mixture was diluted with water and extracted with EtOAc (2×100 mL). The combined organic layers were washed with brine, dried over anhydrous Na₂SO₄, filtered and concentrated under reduced pressure. The crude residue was purified by silica gel column chromatography, eluted with PE/EA (3:2) to afford benzyl 3-(5-methoxy-6-((methoxymethoxy)methyl)pyrazin-2-yl)-4-oxopiperidine-1-carboxylate (850 mg, 13% yield) as a yellow oil. LCMS [M+H]⁺: 416.

### Step 3: Benzyl 4,4-difluoro-3-(5-methoxy-6-((methoxymethoxy)methyl)pyrazin-2-yl)piperidine-1-carboxylate

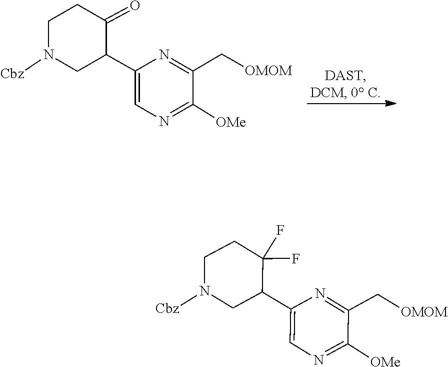

To a stirred solution of benzyl 3-(5-methoxy-6-((methoxymethoxy)methyl)pyrazin-2-yl)-4-oxopiperidine-1-carboxylate (850 mg, 1.6 mmol) in DCM (10 mL) was added DAST (526 mg, 3.3 mmol) dropwise at 0° C. under nitrogen atmosphere. The resulting mixture was stirred at 0° C. for 2 h. The reaction was quenched by the addition of water (10 mL) at 0° C. The resulting mixture was diluted with sat. NaHCO₃ (aq.) (40 mL) and extracted with DCM (2×50 mL). The combined organic layers were washed with brine, dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 40% to 60% gradient in 10 min; detector, UV 254 nm) to afford benzyl 4,4-difluoro-3-(5-methoxy-6-((methoxymethoxy)methyl)pyrazin-2-yl)piperidine-1-carboxylate (210 mg, 29% yield) as a yellow oil. LCMS [M+H]⁺: 438.

### Step 4: [6-(4,4-difluoropiperidin-3-yl)-3-methoxypyrazin-2-yl]methanol

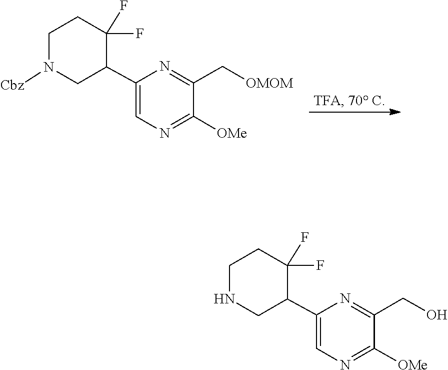

A solution of benzyl 4,4-difluoro-3-(5-methoxy-6-((methoxymethoxy)methyl)pyrazin-2-yl)piperidine-1-carboxylate (210 mg, 0.48 mmol) in TFA (3 mL) was stirred at 70° C. for 1 h under nitrogen atmosphere. The mixture was allowed to cool down to room temperature. The resulting mixture was concentrated under reduced pressure. The crude residue was purified by reversed-phase flash chromatography (conditions: column, C18 silica gel; mobile phase, MeCN in water (0.1% formic acid), 0% to 20% gradient in 10 min; detector, UV 254 nm) to give [6-(4,4-difluoropiperidin-3-yl)-3-methoxypyrazin-2-yl]methanol (110 mg, 88% yield) as a yellow oil. LCMS [M+H]⁺: 260.

### Step 5: (2S)-2-(4,4-difluoro-3-(6-(hydroxymethyl)-5-methoxypyrazin-2-yl)piperidin-1-yl)-N—((R)-5-(3,5-difluorophenyl)-6,7-dihydro-5H-pyrrolo[1,2-a]imidazol-2-yl)propanamide

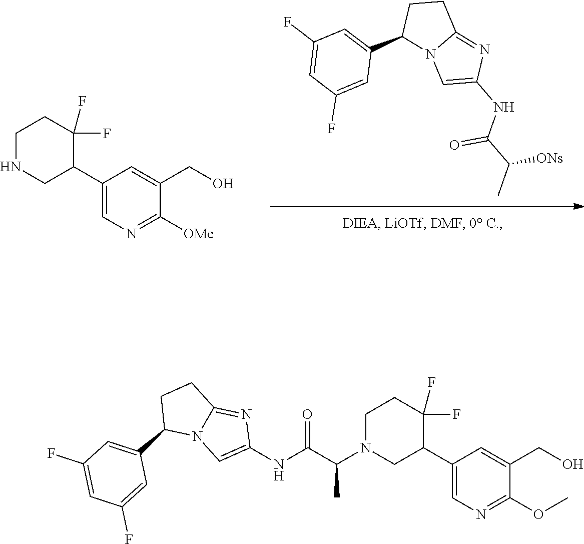

A solution of [6-(4,4-difluoropiperidin-3-yl)-3-methoxypyrazin-2-yl]methanol (110 mg, 0.4 mmol), lithium trifluoromethanesulfonate (165 mg, 1.1 mmol), DIEA (137 mg, 1.1 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (209 mg, 0.4 mmol) in DMF (2 mL) was stirred at 0° C. for 2 days under nitrogen atmosphere. The mixture was directly purified by reversed-phase flash chromatography (conditions: column, C18 silica gel; mobile phase, MeCN in water (0.1% formic acid), 30% to 60% gradient in 10 min; detector, UV 254 nm) to afford (2S)-2-(4,4-difluoro-3-(6-(hydroxymethyl)-5-methoxypyrazin-2-yl)piperidin-1-yl)-N—((R)-5-(3,5-difluorophenyl)-6,7-dihydro-5H-pyrrolo[1,2-a]imidazol-2-yl)propanamide (80 mg, 34% yield) as a yellow oil. LCMS [M+H]⁺: 549.

### Step 6: (2S)-2-(4,4-difluoro-3-(6-(hydroxymethyl)-5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide

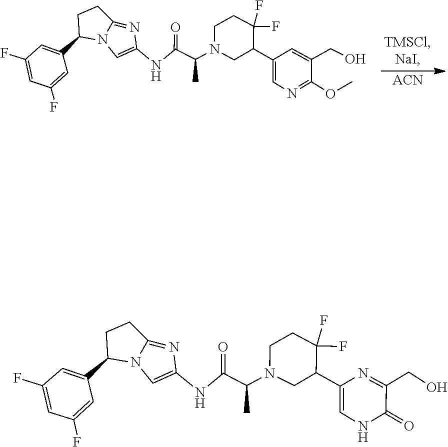

A solution of (2S)-2-(4,4-difluoro-3-(6-(hydroxymethyl)-5-methoxypyrazin-2-yl)piperidin-1-yl)-N—((R)-5-(3,5-difluorophenyl)-6,7-dihydro-5H-pyrrolo[1,2-a]imidazol-2-yl)propanamide (80 mg, 0.15 mmol) in ACN (10 mL) was treated with NaI (109 mg, 0.7 mmol) at 0° C. for 5 min under nitrogen atmosphere. Then, TMSCl (79 mg, 0.7 mmol) was added dropwise at 0° C. The reaction was stirred at 0° C. overnight. The resulting mixture was diluted with water and extracted with EtOAc (2×100 mL). The combined organic layers were washed with brine, dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% formic acid), Mobile Phase B: ACN; Flow rate: 60 mL/min; Gradient (B %): 6% to 25% in 30 min; Wave Length: 254 nm/220 nm; RT1 (min): 11.37 min) to afford (2S)-2-(4,4-difluoro-3-(6-(hydroxymethyl)-5-oxo-4,5-dihydropyrazin-2-yl)piperidin-1-yl)-N—((R)-5-(3,5-difluorophenyl)-6,7-dihydro-5H-pyrrolo[1,2-a]imidazol-2-yl)propanamide (Compound 544) (8.4 mg, 11% yield) as a white solid.

TABLE 51

| Compound# | Characterization Data |
|---|---|
| 544 | LCMS (ESI) [M + H]⁺: 535.4 |
| | ¹H NMR (400 MHz, DMSO-d₆) δ 10.03 (s, 1H), 7.38-7.17 (m, 2H), 6.96-6.92 |
| | (m, 3H), 5.38 (dd, J = 6.6 Hz, 1H), 4.50-4.37 (m, 2H), 3.56-3.45 (m, 2H), |
| | 3.05-2.71 (m, 6H), 2.48-2.26 (m, 2H), 2.20-1.96 (m, 2H), 1.15 (d, J = |
| | 6.9 Hz, 3H). |

### Example 77: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(1,2-dihydroxyethyl)pyridine 1-oxide

### Step 1: tert-butyl 3-(2-chloropyridin-4-yl)-4-oxopiperidine-1-carboxylate

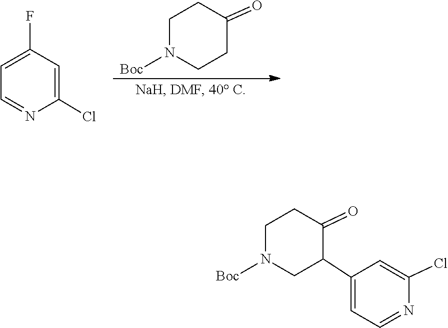 [SMILES: Fc1ccnc(Cl)c1 ; CC(C)(C)OC(=O)N1CCC(=O)CC1 ; CC(C)(C)OC(=O)N1CCC(=O)C(c2ccnc(Cl)c2)C1]

To a mixture of tert-butyl 4-oxopiperidine-1-carboxylate (37.87 g, 190.0 mmol) in DMF (300 mL) was added NaH (15.20 g, 380.1 mmol, 60% in mineral oil) in portions at 0° C. under nitrogen atmosphere. The resulting mixture was stirred at room temperature for 30 min. Then, 2-chloro-4-fluoropyridine (25 g, 190.1 mmol) was added dropwise over 2 min at room temperature. The resulting mixture was stirred at 40° C. for an additional 2 h. The reaction was quenched with sat. NH₄Cl (aq.) at 0° C. The resulting mixture was extracted with EtOAc (3×500 mL). The combined organic layers were washed with brine, dried over anhydrous Na₂SO₄, filtered and concentrated under reduced pressure. The crude residue was purified by silica gel column chromatography, eluted with PE/EA (5:1) to afford tert-butyl 3-(2-chloropyridin-4-yl)-4-oxopiperidine-1-carboxylate (10.1 g, 17% yield) as a light green oil. LCMS [M+H]⁺: 311.

### Step 2: tert-butyl 3-(2-chloropyridin-4-yl)-4,4-difluoropiperidine-1-carboxylate

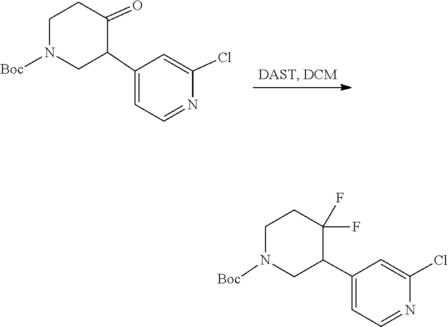 [SMILES: CC(C)(C)OC(=O)N1CCC(=O)C(c2ccnc(Cl)c2)C1 ; CC(C)(C)OC(=O)N1CCC(F)(F)C(c2ccnc(Cl)c2)C1]

To a mixture of tert-butyl 3-(2-chloropyridin-4-yl)-4-oxopiperidine-1-carboxylate (10.1 g, 32.5 mmol) in DCM (100 mL) was added DAST (10.48 g, 65.1 mmol) dropwise at 0° C. under nitrogen atmosphere. The resulting mixture was stirred at room temperature for 3 h. The reaction was quenched by the addition of sat. NaHCO₃ (aq.) (100 mL) at 0° C. The resulting mixture was extracted with CH₂Cl₂ (3×200 mL). The combined organic layers were washed with brine, dried over anhydrous Na₂SO₄, filtered and concentrated under reduced pressure. The crude residue was purified by silica gel column chromatography, eluted with PE/EA (5:1) to afford tert-butyl 3-(2-chloropyridin-4-yl)-4,4-difluoropiperidine-1-carboxylate (4.1 g, 37% yield) as a light yellow oil. LCMS [M+H]⁺: 333.

### Step 3: tert-butyl 4,4-difluoro-3-(2-vinylpyridin-4-yl)piperidine-1-carboxylate

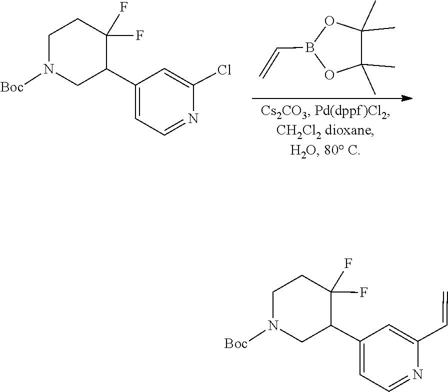

To a mixture of tert-butyl 3-(2-chloropyridin-4-yl)-4,4-difluoropiperidine-1-carboxylate (4.1 g, 12.2 mmol), 2-ethenyl-4,4,5,5-tetramethyl-1,3,2-dioxaborolane (2.09 g, 13.9 mmol) and Cs₂CO₃ (12.09 g, 36.8 mmol) in dioxane (50 mL) and H₂O (10 mL) was added Pd(dppf)Cl₂·CH₂Cl₂ (1.01 g, 1.2 mmol) in portions at room temperature under nitrogen atmosphere. The resulting mixture was stirred at 80° C. for 2 h. The mixture was allowed to cool down to room temperature. The resulting mixture was concentrated under reduced pressure. The crude residue was purified by reversed-phase flash chromatography (conditions: column, C18 silica gel; mobile phase, MeCN in water (0.1% formic acid), 10% to 40% gradient in 20 min; detector, UV 254 nm) to afford tert-butyl 4,4-difluoro-3-(2-vinylpyridin-4-yl)piperidine-1-carboxylate (2.5 g, 62% yield) as a dark green solid. LCMS [M+H]⁺: 325.

### Step 4: tert-butyl 3-(2-(1,2-dihydroxyethyl)pyridin-4-yl)-4,4-difluoropiperidine-1-carboxylate

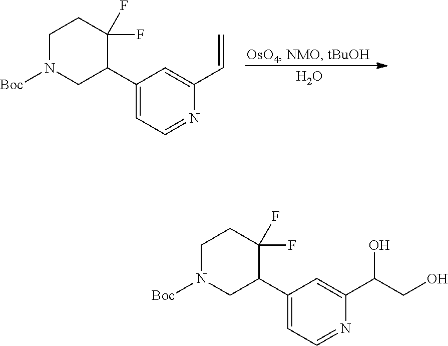 [SMILES: C=Cc1cc(C2CN(C(=O)OC(C)(C)C)CCC2(F)F)ccn1 ; O ; CC(C)(C)OC(=O)N1CCC(F)(F)C(c2ccnc(C(O)CO)c2)C1]

To a mixture of tert-butyl 4,4-difluoro-3-(2-vinylpyridin-4-yl)piperidine-1-carboxylate (2.50 g, 7.7 mmol) and NMO (2.71 g, 23 mmol) in t-BuOH (50 mL) and H₂O (10 mL) was added osmium tetroxide (200 mg, 0.8 mmol) in t-BuOH (1 mL) dropwise at room temperature. The resulting mixture was stirred at room temperature for 3 h. The reaction was quenched by the addition of sat. Na₂S₂O₈ (aq.) (10 mL) at room temperature. The resulting mixture was extracted with EtOAc (3×50 mL). The combined organic layers were washed with brine, dried over anhydrous Na₂SO₄, filtered and concentrated under reduced pressure. The crude residue was purified by silica gel column chromatography, eluted with PE/EA (1:1) to afford tert-butyl 3-(2-(1,2-dihydroxyethyl)pyridin-4-yl)-4,4-difluoropiperidine-1-carboxylate (510 mg, 18% yield).

LCMS [M+H]⁺: 359.

### Step 5: 4-(1-(tert-butoxycarbonyl)-4,4-difluoropiperidin-3-yl)-2-(1,2-dihydroxyethyl)pyridine 1-oxide

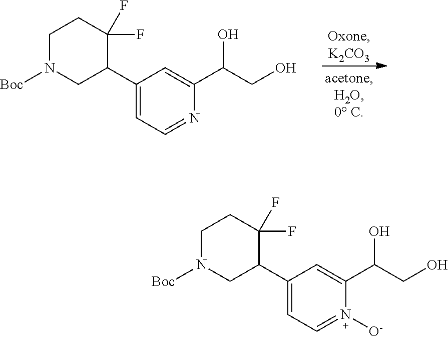

To a mixture of tert-butyl 3-(2-(1,2-dihydroxyethyl)pyridin-4-yl)-4,4-difluoropiperidine-1-carboxylate (510 mg, 1.4 mmol) and K₂CO₃ (393 mg, 2.8 mmol) in acetone (2 mL) and H₂O (2 mL) was added Oxone (1.7 g, 2.8 mmol) in portions at 0° C. under nitrogen atmosphere. The resulting mixture was stirred at 0° C. for 2 h. The mixture was diluted with water (20 mL). The resulting mixture was extracted with EtOAc (3×20 mL). The combined organic layers were washed with brine, dried over anhydrous Na₂SO₄, filtered and concentrated under reduced pressure. The crude residue was purified by silica gel column chromatography, eluted with PE/EA (1:1) to afford 4-(1-(tert-butoxycarbonyl)-4,4-difluoropiperidin-3-yl)-2-(1,2-dihydroxyethyl)pyridine 1-oxide (320 mg, 60% yield). LCMS [M+H]⁺: 375.

### Step 6: 4-(4,4-difluoropiperidin-3-yl)-2-(1,2-dihydroxyethyl)pyridine 1-oxide

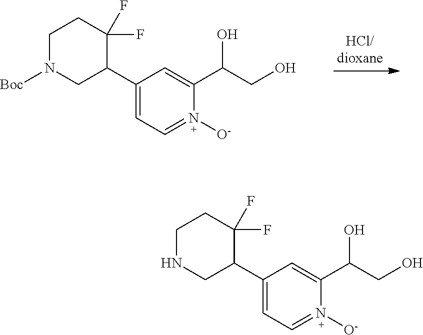

A mixture of 4-(1-(tert-butoxycarbonyl)-4,4-difluoropiperidin-3-yl)-2-(1,2-dihydroxyethyl)pyridine 1-oxide (320 mg, 0.8 mmol) in HCl (5 mL, 4 M in 1,4-dioxane) was stirred at room temperature for 1 h. The resulting mixture was concentrated under reduced pressure. The crude residue was purified by reversed-phase flash chromatography (conditions: column, C18 silica gel; mobile phase, MeCN in water (10 mmol/L NH₄HCO₃), 2% to 5% gradient in 10 min; detector, UV 254 nm) to afford 4-(4,4-difluoropiperidin-3-yl)-2-(1,2-dihydroxyethyl)pyridine 1-oxide (60 mg, 25% yield) as a light yellow solid. LCMS [M+H]⁺: 275.

### Step 7: 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(1,2-dihydroxyethyl)pyridine 1-oxide

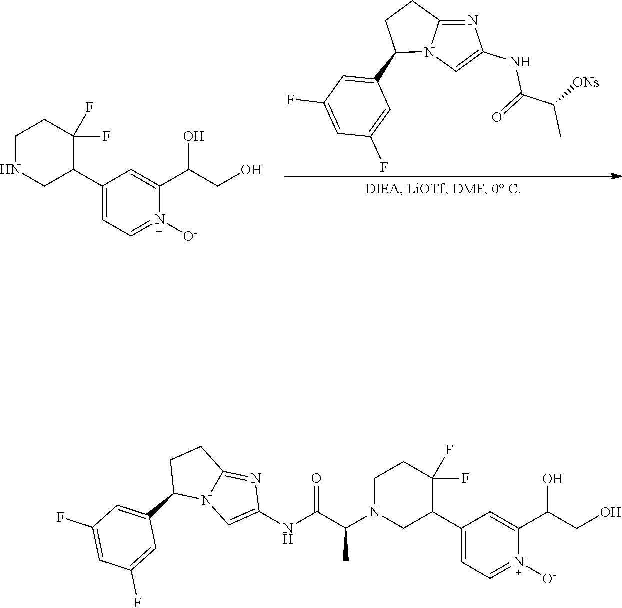

To a mixture of 4-(4,4-difluoropiperidin-3-yl)-2-(1,2-dihydroxyethyl)pyridine 1-oxide (50 mg, 0.2 mmol), DIEA (71 mg, 0.5 mmol) and lithium trifluoromethanesulfonate (85 mg, 0.5 mmol) in DMF (1 mL) was added (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (90 mg, 0.2 mmol) in portions at 0° C. under nitrogen atmosphere. The resulting mixture was stirred at 0° C. for 16 h. The mixture was directly purified by Prep-HPLC with the following conditions (column, C18 silica gel; mobile phase, MeCN in water (10 mmol/L NH₄HCO₃), 10% to 35% gradient in 15 min; detector, UV 254 nm) to afford 4-(1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)-2-(1,2-dihydroxyethyl)pyridine 1-oxide (Compound 545) (2.3 mg, 2.2% yield) as a white solid.

TABLE 52

| Compound# | Characterization Data |
|---|---|
| 545 | LCMS (ESI) [M + H]⁺: 564.2 |
| | ¹H NMR (400 MHz, DMSO) δ 10.11 (s, 1H), 8.17 (d, J = 7.0 Hz, 1H), 7.59-7.50 |
| | (m, 1H), 7.32-7.29 (m, 1H), 7.27-7.20 (m, 1H), 7.00-6.92 (m, 3H), 5.84- |
| | 5.70 (m, 1H), 5.41-5.35 (m, 1H), 5.04 (bs, 1H), 4.95-4.82 (m, 1H), 3.72-3.64 |
| | (m, 1H), 3.05-2.87 (m, 6H), 2.82-2.68 (m, 3H), 2.44-2.35 (m, 2H), 2.17- |
| | 2.01 (m, 2H), 1.22-1.14 (m, 3H). |

### Example 78: (2S)—N—((R)-5-(3,5-difluorophenyl)-6,7-dihydro-5H-pyrrolo[1,2-a]imidazol-2-yl)-2-(3-(5-(1,2-dihydroxyethyl)-6-oxo-1,6-dihydropyridin-3-yl)-4,4-difluoropiperidin-1-yl)propanamide

### Step 1: 1-(5-bromo-2-methoxypyridin-3-yl)-2-((tert-butyldimethylsilyl)oxy)ethan-1-ol

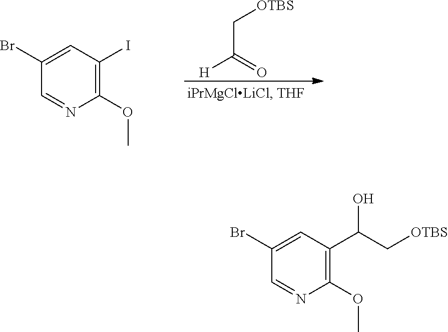

A solution of 5-bromo-3-iodo-2-methoxypyridine (10 g, 31.8 mmol) in THF (200 mL) was stirred at −60° C. for 5 min under nitrogen atmosphere. iPrMgCl·LiCl (28.9 mL, 37.6 mmol, 1.3 M) was added dropwise over 5 min at −60° C. The resulting mixture was stirred at 0° C. for an additional 30 min. Then, 2-[(tert-butyldimethylsilyl)oxy]acetaldehyde (6.61 g, 37.9 mmol) was added dropwise over 10 min at −60° C. The resulting mixture was stirred at room temperature overnight. The reaction was quenched with sat. NH₄Cl (aq.) at room temperature. The resulting mixture was extracted with EtOAc (3×100 mL). The combined organic layers were washed with acetic acid (1×20 mL), dried over anhydrous Na₂SO₄, filtered and concentrated under reduced pressure to afford 1-(5-bromo-2-methoxypyridin-3-yl)-2-((tert-butyldimethylsilyl)oxy)ethan-1-ol (6.7 g, 58% yield) as a yellow liquid, which was used in the next step without further purification. LCMS [M+H]⁺: 362.

### Step 2: 1-(5-bromo-2-methoxypyridin-3-yl)ethane-1,2-diol

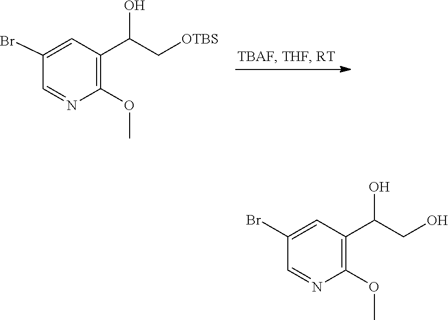 [SMILES: COc1ncc(Br)cc1C(O)CO[Si](C)(C)C(C)(C)C ; COc1ncc(Br)cc1C(O)CO]

A solution of 1-(5-bromo-2-methoxypyridin-3-yl)-2-((tert-butyldimethylsilyl)oxy)ethan-1-ol (6.65 g, 18.3 mmol) and TBAF (36.6 mL, 36.6 mmol, 1 M in THF) in THF (100 mL) was stirred at room temperature for 2 h. The reaction was diluted with water at room temperature. The resulting mixture was extracted with EtOAc (3×100 mL). The combined organic layers were washed with brine, dried over anhydrous Na₂SO₄, filtered and concentrated under reduced pressure. The crude residue was purified by silica gel column chromatography, eluted with PE/EA (1:1) to afford 1-(5-bromo-2-methoxypyridin-3-yl)ethane-1,2-diol (2.1 g, 46% yield) as a yellow oil. LCMS [M+H]⁺: 248.

### Step 3: 5-bromo-3-(2,4,7,9-tetraoxadecan-5-yl)-2-methoxypyridine

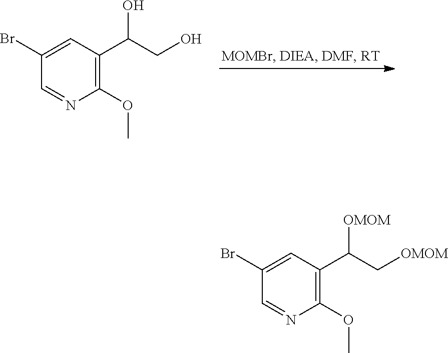

A solution of 1-(5-bromo-2-methoxypyridin-3-yl)ethane-1,2-diol (2.08 g, 8.4 mmol) in DCM (30 mL) was treated with DIEA (7.6 g, 58.8 mmol) at 0° C. for 5 min under nitrogen atmosphere. Bromo(methoxy)methane (5.2 g, 41.6 mmol) was added dropwise at 0° C. The resulting mixture was stirred at room temperature overnight. The reaction was quenched by the addition of water (100 mL) at room temperature. The resulting mixture was extracted with EtOAc (3×100 mL). The combined organic layers were washed with brine, dried over anhydrous Na₂SO₄, filtered and concentrated under reduced pressure. The crude residue was purified by silica gel column chromatography, eluted with PE/EA (1:1) to afford 5-bromo-3-(2,4,7,9-tetraoxadecan-5-yl)-2-methoxypyridine (1.5 g, 53% yield) as a yellow solid. LCMS [M+H]⁺: 336.

### Step 4: tert-butyl 3-(5-(2,4,7,9-tetraoxadecan-5-yl)-6-methoxypyridin-3-yl)-4-oxopiperidine-1-carboxylate

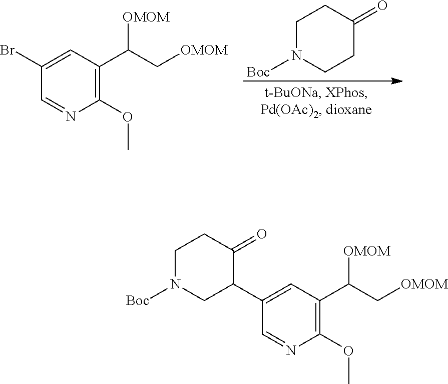

A solution of 5-bromo-3-(2,4,7,9-tetraoxadecan-5-yl)-2-methoxypyridine (1.49 g, 4.4 mmol), tert-butyl 4-oxopiperidine-1-carboxylate (1.05 g, 5.2 mmol), t-BuONa (1.27 g, 13.2 mmol), XPhos (440 mg, 0.9 mmol) and Pd(OAc)₂ (69 mg, 0.3 mmol) in dioxane (20 mL) was stirred at 50° C. for 2 h under nitrogen atmosphere. The mixture was allowed to cool down to room temperature. The reaction was diluted with water. The resulting mixture was extracted with EtOAc (3×50 mL). The combined organic layers were washed with brine, dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 50% to 70% gradient in 10 min; detector, UV 254 nm) to afford tert-butyl 3-(5-(2,4,7,9-tetraoxadecan-5-yl)-6-methoxypyridin-3-yl)-4-oxopiperidine-1-carboxylate (1.32 g, 65% yield) as a yellow oil. LCMS [M+H]⁺: 455.

### Step 5: tert-butyl 3-(5-(2,4,7,9-tetraoxadecan-5-yl)-6-methoxypyridin-3-yl)-4,4-difluoropiperidine-1-carboxylate

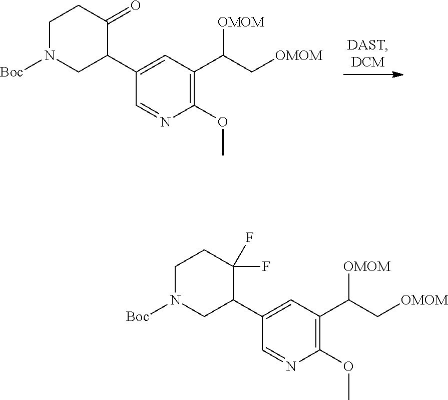

DAST (2.34 g, 14.5 mmol) was added to a solution of tert-butyl 3-(5-(2,4,7,9-tetraoxadecan-5-yl)-6-methoxypyridin-3-yl)-4-oxopiperidine-1-carboxylate (1.32 g, 2.9 mmol) in DCM (20 mL) at 0° C. for 5 min under nitrogen atmosphere. The resulting mixture was stirred at 0° C. for 1 h. The reaction was quenched with sat. Na₂CO₃ (aq.) at 0° C. The resulting mixture was extracted with EtOAc (3×50 mL). The combined organic layers were washed with brine, dried over anhydrous Na₂SO₄, filtered and concentrated under reduced pressure. The crude residue was purified by reversed-phase flash chromatography (conditions: column, C18 silica gel; mobile phase, MeCN in water (0.1% formic acid), 55% to 70% gradient in 10 min; detector, UV 254 nm) to afford tert-butyl 3-(5-(2,4,7,9-tetraoxadecan-5-yl)-6-methoxypyridin-3-yl)-4,4-difluoropiperidine-1-carboxylate (450 mg, 32% yield) as a yellow oil. LCMS [M+H]⁺: 499.

### Step 6: 1-(5-(4,4-difluoropiperidin-3-yl)-2-methoxypyridin-3-yl)ethane-1,2-diol

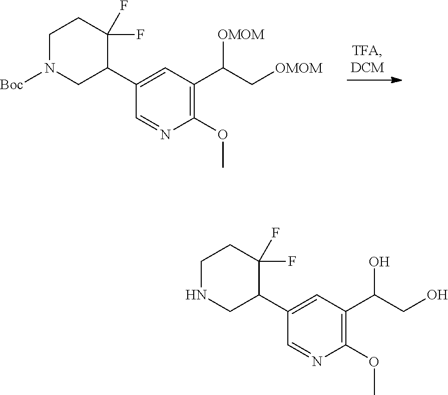

A solution of tert-butyl 3-(5-(2,4,7,9-tetraoxadecan-5-yl)-6-methoxypyridin-3-yl)-4,4-difluoropiperidine-1-carboxylate (400 mg, 0.8 mmol) in DCM (3 mL) and TFA (3 mL) was stirred at room temperature overnight. The resulting mixture was concentrated under reduced pressure. The crude residue was purified by reversed-phase flash chromatography (conditions: column, C18 silica gel; mobile phase, MeCN in water (10 mmol/L NH₄HCO₃), 10% to 20% gradient in 10 min; detector, UV 254 nm) to afford 1-(5-(4,4-difluoropiperidin-3-yl)-2-methoxypyridin-3-yl)ethane-1,2-diol (170 mg, 70% yield) as a white oil. LCMS [M+H]⁺: 289.

### Step 7: (2S)—N—((R)-5-(3,5-difluorophenyl)-6,7-dihydro-5H-pyrrolo[1,2-a]imidazol-2-yl)-2-(3-(5-(1,2-dihydroxyethyl)-6-methoxypyridin-3-yl)-4,4-difluoropiperidin-1-yl)propanamide

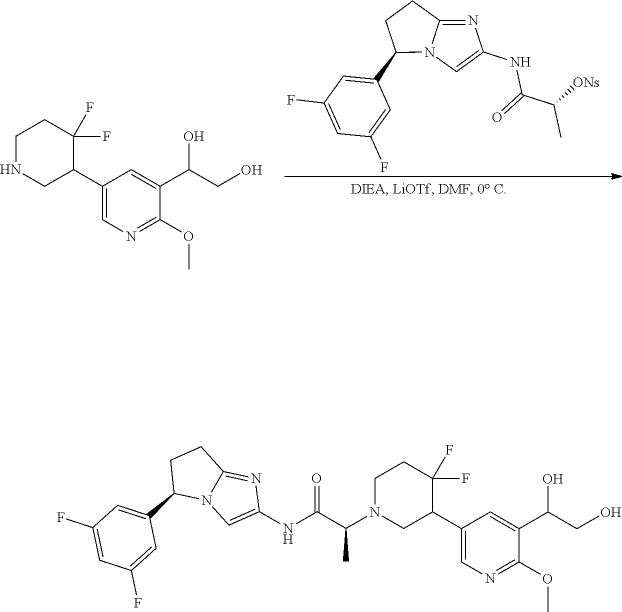

To a stirred solution of 1-(5-(4,4-difluoropiperidin-3-yl)-2-methoxypyridin-3-yl)ethane-1,2-diol (170 mg, 0.6 mmol), DIEA (227 mg, 1.8 mmol) and lithium trifluoromethanesulfonate (272 mg, 1.7 mmol) in DMF (5 mL) was added (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (354 mg, 0.7 mmol) dropwise at 0° C. under nitrogen atmosphere. The resulting mixture was stirred at 0° C. for 2 days. The mixture was directly purified by reversed-phase flash chromatography (conditions: column, C18 silica gel; mobile phase, MeCN in water (0.1% formic acid), 30% to 50% gradient in 10 min; detector, UV 254 nm) to afford (2S)—N—((R)-5-(3,5-difluorophenyl)-6,7-dihydro-5H-pyrrolo[1,2-a]imidazol-2-yl)-2-(3-(5-(1,2-dihydroxyethyl)-6-methoxypyridin-3-yl)-4,4-difluoropiperidin-1-yl)propanamide (65 mg, 19% yield) as a white oil. LCMS [M+H]⁺: 578.

### Step 8: (2S)—N—((R)-5-(3,5-difluorophenyl)-6,7-dihydro-5H-pyrrolo[1,2-a]imidazol-2-yl)-2-(3-(5-(1,2-dihydroxyethyl)-6-oxo-1,6-dihydropyridin-3-yl)-4,4-difluoropiperidin-1-yl)propanamide

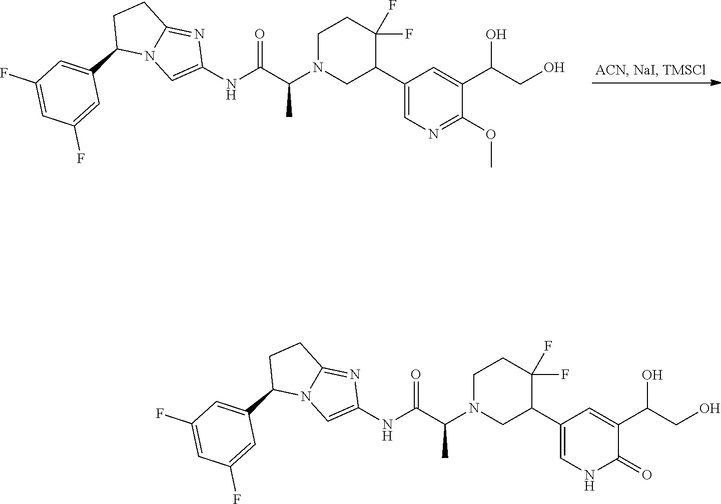 [SMILES: COc1ncc(C2CN([C@@H](C)C(=O)Nc3cn4c(n3)CC[C@@H]4c3cc(F)cc(F)c3)CCC2(F)F)cc1C(O)CO ; C[C@@H](C(=O)Nc1cn2c(n1)CC[C@@H]2c1cc(F)cc(F)c1)N1CCC(F)(F)C(c2c[nH]c(=O)c(C(O)CO)c2)C1]

A solution of (2S)—N—((R)-5-(3,5-difluorophenyl)-6,7-dihydro-5H-pyrrolo[1,2-a]imidazol-2-yl)-2-(3-(5-(1,2-dihydroxyethyl)-6-methoxypyridin-3-yl)-4,4-difluoropiperidin-1-yl)propanamide (65 mg, 0.1 mmol) in ACN (2 mL) was treated with NaI (78 mg, 0.5 mmol) at 0° C. for 5 min under nitrogen atmosphere. Then, TMSCl (65 mg, 0.6 mmol) was added dropwise at 0° C. The resulting mixture was stirred at room temperature for 4 h. The reaction was quenched with water at 0° C. The resulting mixture was washed with EtOAc (3×20 mL). The aqueous layer was concentrated under reduced pressure. The crude residue was purified by Prep-HPLC with the following conditions (Column: Xselect CSH Prep C18, 30*150 mm 5 μm; Mobile Phase A: Water (0.1% formic acid), Mobile Phase B: ACN; Flow rate: 60 mL/min; Gradient (B %): 7% to 27% in 14 min; Wave Length: 254 nm/220 nm; RT1 (min): 12.67 min) to afford (2S)—N—((R)-5-(3,5-difluorophenyl)-6,7-dihydro-5H-pyrrolo[1,2-a]imidazol-2-yl)-2-(3-(5-(1,2-dihydroxyethyl)-6-oxo-1,6-dihydropyridin-3-yl)-4,4-difluoropiperidin-1-yl)propanamide (Compound 546) (27.1 mg, 43% yield) as a white solid.

TABLE 53

| Compound# | Characterization Data |
|---|---|
| 546 | LCMS (ESI) [M + H]⁺: 564.2 |
| | ¹H NMR (400 MHz, DMSO) δ 11.62 (bs, 1H), 10.29 (bs, 1H), 7.45 (s, 1H), 7.30- |
| | 7.19 (m, 2H), 7.09-6.90 (m, 3H), 5.43 (s, 1H), 4.60 (s, 1H), 3.61-3.51 (m, 2H), |
| | 3.24-3.16 (m, 3H), 3.10-2.90 (m, 4H), 2.90-2.70 (m, 3H), 2.49-2.35 (m, |
| | 2H), 2.30-2.00 (m, 2H), 1.48-1.05 (m, 3H). |

### Example 79: 4-((3S)-1-(1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide

### Step 1: 4-((3S)-1-((2S)-1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide

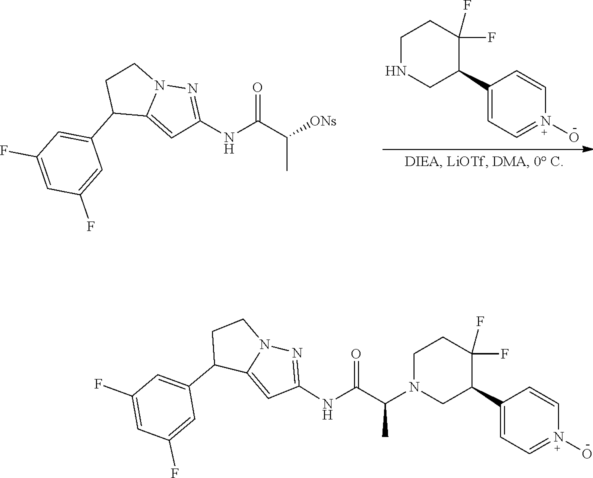

A solution of (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (80 mg, 0.2 mmol), (S)-4-(4,4-difluoropiperidin-3-yl)pyridine 1-oxide (42 mg, 0.2 mmol), DIEA (52 mg, 0.4 mmol) and lithium trifluoromethanesulfonate (65 mg, 0.4 mmol) in DMF (3 mL) was stirred at 0° C. for 3 days under nitrogen atmosphere. The resulting mixture was concentrated under reduced pressure. The crude residue was directly purified by reversed-phase flash chromatography (conditions: column, C18 silica gel; mobile phase, ACN in water (0.1% formic acid), 0% to 100% gradient in 20 min; detector, UV 254 nm) to afford 4-((3S)-1-((2S)-1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (40 mg, 49% yield) as a light yellow solid. LCMS (ESI) [M+H]⁺: 504.

### Step 2: 4-((S)-1-((R)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide and 4-((S)-1-((R)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide and 4-((S)-1-((S)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide and 4-((S)-1-((S)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide

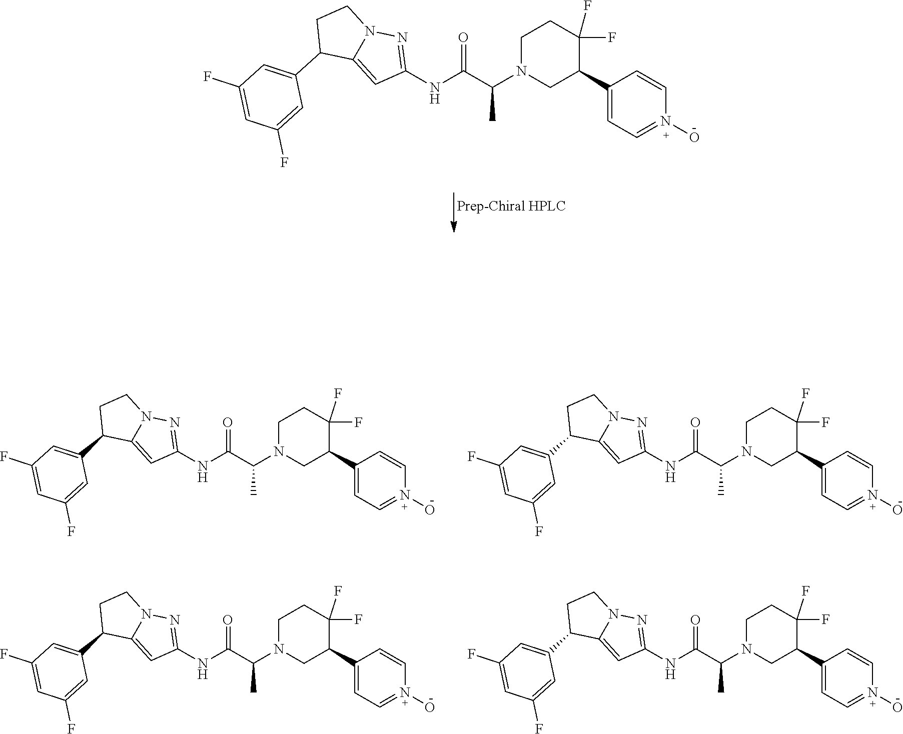

4-((3S)-1-((2S)-1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (40 mg) was separated by Prep-Chiral HPLC with the following conditions (Column: Lux 5 μM Cellulose-2, 30*250 mm, 5.0 μm; Mobile Phase A: Hex (10 mM NH₃-MeOH), Mobile Phase B: EtOH:ACN=5:1; Flow rate: 40 mL/min; Gradient (B %): isocratic 50% B; Wave Length: 260/280 nm; RT1 (min): 11.1; RT2 (min): 19), to provide two fractions each as a mixture of diastereomers as partial racemization occurred during the purification process. The first fraction was separated by Prep-Chiral HPLC with the following conditions (Column: CHIRALPAK IF 3*25 cm, 5 μm; Mobile Phase A: MtBE (10 mM NH₃-MeOH), Mobile Phase B: MEOH; Flow rate: 40 mL/min; Gradient (B %): isocratic 45% B; Wave Length: 212/270 nm; RT1 (min): 4.921; RT2 (min): 5.644) to afford first peak 4-((S)-1-((R)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (Compound 547) (Method AQ, Peak 2, 2.64 min; 1.1 mg, 100% d.e.) as a light yellow oil and second peak 4-((S)-1-((R)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (Compound 548) (Method AQ, Peak 1, 1.15 min; 1.6 mg, 100% d.e.) as a light yellow oil.

The second fraction was separated by Prep-Chiral HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient (B %): isocratic 50% B; Wave Length: 206/232 nm; RT1 (min): 11.5; RT2 (min): 16.5) to afford first peak 4-((S)-1-((S)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (Compound 549) (Method AQ, Peak 2, 2.48 min; 4.4 mg, 100% d e.) as a light yellow oil and second peak 4-((S)-1-((S)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide (Compound 550) (Method AQ, Peak 1, 2.06 min; 6.9 mg, 100% d e.) as a light yellow oil.

TABLE 54

| Compound# | Characterization Data |
|---|---|
| 547 | LCMS (ESI) [M + H]⁺: 504.2 |
| | ¹H NMR (400 MHz, DMSO-d₆) δ 10.26 (s, 1H), 8.16 (d, J = 7.2 Hz, 2H), 7.39 (d, J = 6.6 Hz, |
| | 2H), 7.17-7.10 (m, 1H), 7.02-6.97 (m, 2H), 6.24 (s, 1H), 4.57-4.48 (m, 1H), 4.27-4.15 |
| | (m, 1H), 4.08-3.95 (m, 1H), 3.58-3.44 (m, 2H), 3.08-2.90 (m, 3H), 2.77-2.62 (m, 2H), |
| | 2.46-2.37 (m, 1H), 2.17-2.01 (m, 2H), 1.20 (d, J = 6.9 Hz, 3H). |
| 548 | LCMS (ESI) [M + H]⁺: 504.2 |
| | ¹H NMR (400 MHz, DMSO-d₆) δ 10.29 (s, 1H), 8.16 (d, J = 7.2 Hz, 2H), 7.38 (d, J = 6.7 Hz, |
| | 2H), 7.14-7.11 (m, 1H), 7.02-6.96 (m, 2H), 6.26 (s, 1H), 4.59-4.51 (m, 1H), 4.25-4.16 |
| | (m, 1H), 4.09-3.96 (m, 1H), 3.57-3.45 (m, 2H), 3.07-2.89 (m, 3H), 2.77-2.59 (m, 2H), |
| | 2.48-2.32 (m, 1H), 2.19-2.03 (m, 2H), 1.20 (d, J = 6.9 Hz, 3H). |
| 549 | LCMS (ESI) [M + H]⁺: 504.2 |
| | ¹H NMR (400 MHz, DMSO-d₆) δ 10.28 (s, 1H), 8.16 (d, J = 7.2 Hz, 2H), 7.38-7.34 (m, 2H), |
| | 7.18-7.10 (m, 1H), 7.05-6.95 (m, 2H), 6.26 (s, 1H), 4.58-4.49 (m, 1H), 4.27-4.16 (m, |
| | 1H), 4.10-3.98 (m, 1H), 3.62-3.41 (m, 2H), 3.11-2.95 (m, 2H), 2.94-2.86 (m, 2H), |
| | 2.49-2.30 (m, 2H), 2.17-1.97 (m, 2H), 1.18 (d, J = 6.9 Hz, 3H). |
| 550 | LCMS (ESI) [M + H]⁺: 504.2 |
| | ¹H NMR (400 MHz, DMSO-d₆) δ 10.26 (s, 1H), 8.16 (d, J = 7.2 Hz, 2H), 7.37 (d, J = 6.8 Hz, |
| | 2H), 7.18-7.09 (m, 1H), 7.06-6.97 (m, 2H), 6.24 (s, 1H), 4.53-4.49 (m, 1H), 4.27-4.18 |
| | (m, 1H), 4.09-4.00 (m, 1H), 3.63-3.40 (m, 2H), 3.10-2.93 (m, 2H), 2.92-2.88 (m, 2H), |
| | 2.48-2.38 (m, 2H), 2.16-1.98 (m, 2H), 1.18 (d, J = 6.9 Hz, 3H). |

### Biological Examples

The utility of the compounds of the present invention and the salts of such compounds as medical agents in the treatment of the above described disease/conditions in mammals (e.g. humans, male or female) is demonstrated by the activity of the compounds of the present invention in one or more of the conventional assays and in vivo assays described and noted below. The in vivo assays (with appropriate modifications within the skill in the art) can be used to determine the activity of other agents as well as the compounds of the present invention. Thus, the protocols described below can also be used to demonstrate the utility of the combinations of the compounds of the present invention. The assays and models may also demonstrate particular other property advantages e.g., side effect profile; half-life. In addition, such assays provide a means whereby the activities of the compounds of the present invention and the salts of such compounds (or the other agents described herein) can be compared to each other and with the activities of other known compounds. The results of these comparisons are useful for determining dosage levels in mammals, including humans, for the treatment of such diseases.

Absorption, Distribution, Metabolism and Excretion (ADME) and pharmacokinetics (PK) of compounds and exemplary assays are discussed in the on-line publication by Thomas D. Y. Chung, David B. Terry and Layton H. Smith “In Vitro and In Vivo Assessment of ADME and PK Properties During Lead Selection and Lead Optimization-Guidelines, Benchmarks and Rules of Thumb-(https://www.ncbi.nlm.nih.gov/books/NBK326710/) Exemplary in vivo animal models of atopic dermatitis are included in J. Invest Dermal. 2009 January: 129(1): 31-40. Exemplary pruritus mouse models are described in Front Med (Lausanne) 2021 Feb. 23; 8:630237 “Comparative Study on Different Skin Pruritus Mouse Models.” Exemplary in vivo animal models of inflammation are included in Int. J. Mol. Si 2019 September; 20(18): 4367. Exemplary in vivo models of autoimmune disease/conditions are included in Autoimmun Rev. 2018 May; 17(5): 473-479. Exemplary in vitro assays of cancer are described in Front Bioeng Biotechnol 2016; 4:12 “In Vitro Tumore Models: Advantages, Disadvantages, Variables, and Selecting the Right Platform.” Exemplary in vivo animal models of cancer are described in Front Oncol, 2019; 8: 429 “Next-Generation in vivo Modeling of Human Cancers. Exemplary in vivo animal models of pain are described in Journal of Neuroscience Methods Volume 348, January 2021, 108997, “Animal models of pain: Diversity and benefits” and J. Pain. 2013 November: 14(11): 10.1016/j.jpain.2013.06.008 “An overview of animal models of pain: disease models and outcome measures”.

The following protocols may of course be varied by those skilled in the art.

### MRGPRX2 Activity Assay:

FlpIn TRex-MRGPRX2-HEK293 cells (herein TRex-MRPGRX2-HEK) stably transfected to express human MRGPRX2 were maintained at 37° C. with 5% CO₂ and grown in DMEM supplemented with 10% FBS, 1% L-Glutamate, 1% penicillin/streptomycin, 100 ug/mL Hygromycin B, and 15 ug/mL Blasticidin.

GOI (MRGPRX2) expression was induced with 100 ng/mL of Doxycycline 18-24 hours prior to experiment, in flask. On the day of experiment, cells were lifted with TripLE Express and seeded in 384-well white walled opaque bottomed, assay plates, at 8,000 cells per well in 5 uL freshly made assay buffer (HBSS, 20 mM HEPES, 0.1% BSA and 10 mM LiCl). Compounds solubilized in 100% DMSO at 30 mM were serially diluted (1:4) first in 100% DMSO, and an intermediate plate of assay buffer was used to establish a final top concentration of 30 uM (in assay plate). Final DMSO concentration was kept constant at 1% across assay plates. Cortistatin-14, at 30 mM stock concentration was solubilized in HBSS+0.1% BSA, stored at −20° C. in 5 μL aliquots and discarded after one freeze-thaw cycle. Final concentration of agonist used in antagonist mode assay was 1 μM, estimated EC₈₀ (EC₅₀ of Cortistatin-14˜2.5e-7 M).

Antagonist equilibrium was established with 30 minutes preincubation, followed by no wash, direct addition, and stimulation of agonist for 1 hour at 37° C./5% CO₂. IP-1 standards and HTRF detection reagents were added according to the IP-one Gq kit protocol purchased from CisBio (part number 62IPAPEJ). The plate was read on an EnVision XCite plate reader. The HTRF ratio was then calculated from raw data (channel1/channel2×10,000) and analyzed using either GraphPad Prism or a custom Python script to calculate IC₅₀ using Log[Antagonist] vs. response (4 parameter) nonlinear regression.

Activity data for selected MRGPRX2 antagonist (versus 1 μM Cortistatin-14 agonist) are displayed in the table below.

### LAD2 Degranulation Assay (Protocol 1):

LAD-2 cells were maintained at 37° C. with 5% CO₂ and grown in complete StemPro-34 media supplemented with 1% L-Glutamate, 1% penicillin/streptomycin, and 250 ng/mL human SCF.

On the day of experiment, cells were collected by centrifugation and seeded in 384-well assay plates, at 4,000 cells per well in 10 μl freshly made assay buffer (10 mM HEPES, 130 mM NaCl, 5 mM KCl, 1.8 mM CaCl₂), 1.0 mM MgCl₂, 5.6 mM glucose, 0.1% BSA, pH=7.40). For dose-response assays, compounds solubilized in 100% DMSO at 30 mM were serially diluted (1:3) first in 100% DMSO, and 2 intermediate dilutions with assay buffer were used to establish a final top concentration of 3 μM (in assay plate). Final DMSO concentration was kept constant at 0.1% across plates for all assays. Cortistatin-14 at 30 mM stock concentration was solubilized in HBSS+0.1% BSA, stored at −20° C. in 5 μl aliquots and discarded after one freeze-thaw cycle. Final concentration of agonist used in both dose-response and single point assays was 1 μM, prepared in assay buffer.

Antagonist equilibrium was established with 30 minute preincubation, followed by no wash, direct addition, and stimulation of agonist for 30 minutes at 37° C. For detection of β-hexosaminidase activity, assay plates were centrifuged and 5 μl of cell-free supernatant from each assay well was transferred to a black-walled black-bottomed 384-well plate. Substrate solution (1 mM 4-Methylumbelliferyl N-acetyl-β-D-glucosaminide in citrate buffer, pH=4.5, 15 μl) was added to each well and plates were incubated for 1 hour at 37° C. The plate was read on an EnVision XCite plate reader for umbelliferone fluorescence intensity (λex=355 nm, λem=460 nm). Background-subtracted data was analyzed using either GraphPad Prism or a custom Python script to calculate IC₅₀ using either Log[Antagonist] vs. response (4 parameter) nonlinear regression (dose-response assays).

### LAD2 Degranulation Assay (Protocol 2):

LAD-2 cells were maintained at 37° C. with 5% CO₂ and grown in complete StemPro-34 media supplemented with 1% L-Glutamate, 1% penicillin/streptomycin, and 250 ng/mL human SCF.

18-24 hours prior to experiment, LAD-2 cells were collected and transferred to growth media without SCF. On the day of experiment, cells were collected by centrifugation and seeded in 384-well assay plates, at 4,000 cells per well in 17 μl freshly made assay buffer (10 mM HEPES, 137 mM NaCl, 2.7 mM KCl, 1.8 mM CaCl₂), 1.0 mM MgCl₂, 5.6 mM glucose, 0.4 mM NaH₂PO₄, 0.04% BSA, pH=7.40). For dose-response assays, compounds solubilized in 100% DMSO at 30 mM were serially diluted (1:3) first in 100% DMSO, and 2 intermediate dilutions with assay buffer were used to establish a final top concentration of 3 μM (in assay plate). For single point assays, dilution of DMSO stocks to 250 nM (in assay plate) was performed in the same manner as described for dose-response assays. Final DMSO concentration was kept constant at 0.1% across plates for all assays. Cortistatin-14 at 30 mM stock concentration was solubilized in HBSS+0.1% BSA, stored at −20° C. in 5 μl aliquots and discarded after one freeze-thaw cycle. Final concentration of agonist used in both dose-response and single point assays was 0.5 μM, prepared in assay buffer.

Antagonist equilibrium was established with 2 h preincubation, followed by no wash, direct addition, and stimulation of agonist for 30 minutes at 37° C. For detection of β-hexosaminidase activity, assay plates were centrifuged and 5 μl of cell-free supernatant from each assay well was transferred to a black-walled black-bottomed 384-well plate. Substrate solution (1 mM 4-Methylumbelliferyl N-acetyl-β-D-glucosaminide in citrate buffer, pH=4.5, 15 Ml) was added to each well and plates were incubated for 1 hour at 37° C. The plate was read on an EnVision XCite plate reader for umbelliferone fluorescence intensity (λex=355 nm, λem=460 nm). Background-subtracted data was analyzed using either GraphPad Prism or a custom Python script to calculate IC₅₀ using either Log[Antagonist] vs. response (4 parameter) nonlinear regression (dose-response assays) or % control β-hexosaminidase activity (single point assays).

TABLE 55

| | | LAD2 | LAD2 | LAD2 |
|---|---|---|---|---|
| | MRGPRX2 | Degranulation | Degranulation | Degranulation |
| Compound | Activity Assay | Assay (Protocol 1) | Assay (Protocol 2) | Assay (Protocol 2) |
| # | IC50 (nM) | IC50 (nM) | IC50 (nM) | % Control |
| 1 | 58 | 1940 | | |
| 2 | 2950 | | | |
| 3 | >10000 | | | |
| 4 | >10000 | | | |
| 5 | 9 | 14 | | |
| 6 | 1780 | | | |
| 7 | 45 | >3000 | | |
| 8 | 9290 | | | |
| 9 | 17 | 28 | | |
| 10 | 1,610 | | | |
| 11 | 3.7 | 12 | | |
| 12 | 196 | | | |
| 13 | 233 | | | |
| 14 | 465 | | | |
| 15 | >10000 | | | |
| 16 | >10000 | | | |
| 17 | 125 | | | |
| 18 | >10000 | | | |
| 19 | 2.1 | 1.6 | | |
| 20 | 193 | | | |
| 21 | 3.3 | 0.7 | | |
| 22 | 124 | | | |
| 23 | 700 | | | |
| 24 | 3910 | | | |
| 25 | >10000 | | | |
| 26 | 13 | 40 | | |
| 27 | 587 | | | |
| 28 | 2.1 | 19 | | |
| 29 | 220 | 212 | | |
| 30 | 258 | | | |
| 31 | 361 | | | |
| 32 | 104 | | | |
| 33 | 30 | | | |
| 34 | 11 | 25 | | |
| 35 | 7.9 | 57 | | |
| 36 | 209 | | | |
| 37 | 54 | | | |
| 38 | 93 | | | |
| 39 | 4.1 | 3.3 | | |
| 40 | 25 | | | |
| 41 | 269 | | | |
| 42 | 169 | | | |
| 43 | 131 | | | |
| 44 | 241 | | | |
| 45 | 885 | | | |
| 46 | 210 | | | |
| 47 | 775 | | | |
| 48 | 214 | | | |
| 49 | 53 | | | |
| 50 | 45 | | | |
| 51 | 34 | | | |
| 52 | 22 | | | |
| 53 | 783 | 1020 | | |
| 54 | 432 | | | |
| 55 | 4.0 | 22 | | |
| 56 | 76 | 976 | | |
| 57 | >10000 | | | |
| 58 | >10000 | | | |
| 59 | 432 | | | |
| 60 | >30000 | | | |
| 61 | 477 | | | |
| 62 | 267 | | | |
| 63 | 114 | | | |
| 64 | 78 | | | |
| 65 | 504 | | | |
| 66 | 71 | | | |
| 67 | 4330 | | | |
| 68 | 23 | 30 | | |
| 69 | >30000 | | | |
| 70 | >30000 | | | |
| 71 | 2360 | | | |
| 72 | 12 | 20 | | |
| 73 | 8560 | | | |
| 74 | >30000 | | | |
| 75 | 513 | | | |
| 76 | 2220 | | | |
| 77 | 18 | 51 | | |
| 78 | >30000 | | | |
| 79 | 18100 | | | |
| 80 | 755 | >3000 | | |
| 81 | >30000 | | | |
| 82 | 1170 | | | |
| 83 | 8 | 13 | 12 | |
| 84 | 12700 | | | |
| 85 | 28 | 100 | | |
| 486 | 1180 | | | |
| 487 | 13800 | | | |
| 488 | 8810 | | | |
| 489 | 13 | | 9.5 | 14 |
| 490 | 3.5 | | 34 | |
| 491 | 1640 | | | |
| 492 | 35 | | | |
| 493 | >30000 | | >3000 | |
| 494 | 20 | | 6.9 | 13 |
| 495 | >30000 | | | |
| 496 | 3470 | | | |
| 497 | >30000 | | | |
| 498 | 686 | | 79 | |
| 499 | >30000 | | >3000 | |
| 500 | >30000 | | >3000 | |
| 501 | | | | 101 |
| 502 | | | | 44 |
| 503 | | | | 105 |
| 504 | | | 31 | 13 |
| 505 | | | >3000 | 106 |
| 506 | | | 30 | |
| 507 | | | | 130 |
| 508 | | | | 110 |
| 509 | | | | 11 |
| 510 | | | | 87 |
| 511 | >30000 | | | |
| 512 | 4080 | | | |
| 513 | >30000 | | | |
| 514 | >30000 | | | |
| 515 | 4600 | | | |
| 516 | >30000 | | | |
| 517 | >30000 | | | |
| 518 | >30000 | | | |
| 519 | >30000 | | | |
| 520 | 129 | | | |
| 521 | 4750 | | | |
| 522 | 229 | | | |
| 523 | >30000 | | | |
| 524 | 50 | | | |
| 525 | 5480 | | | |
| 526 | 9.8 | | | |
| 527 | 102 | | | |
| 528 | 6.8 | | | |
| 529 | 80 | | | |
| 530 | | | 0.81 | |
| 531 | 592 | | 614 | |
| 532 | 5200 | | | |
| 533 | 18 | | | |
| 534 | >30000 | | | |
| 535 | 31 | | | |
| 536 | | | | 89 |
| 537 | | | | 10 |
| 538 | | | | 81 |
| 539 | | | | 12 |
| 540 | | | | 124 |
| 541 | | | | 11 |
| 542 | | | | 126 |
| 543 | | | | 59 |
| 544 | | | | 81 |
| 545 | | | | 16 |
| 546 | | | | 34 |
| 549 | 439 | | | |
| 550 | 6 | | | |

hERG Safety Evaluation by Manual Patch-Clamp System:

A stable BIEK 293 cell line with inducible hERG channel expression (hERG-T-Rex 293, Invitrogen cat #K1236) was cultured in medium containing 85% DMVEM, 10% dialyzed FBS, 0.1 mM NEAA, 25 mM HEPES, 100 U/mL Penicillin-Streptomycin, 5 μg/mL Blasticidin and 400 μg/mL Geneticin. Before the assay, cells were induced with doxycycline at 1 μg/mL for 48 hours. On the experiment day, the induced cells were resuspended and plated onto coverslips at 5×10⁵ cells/per 3.5 cm cell culture dish prior to use and cultured in medium without Blasticidin and Geneticin.

Electrophysiology recordings were performed in extracellular solution (ECS) containing (in mM): 132 NaCl, 4 KCl, 3 CaCl₂), 0.5 MgCl₂, 11.1 D-(+) glucose, and 10 HEPES (pH adjusted to 7.35 with NaOH, osmolarity 285-295 mOsm). Recording pipets contained (in mM) 10 NaCl, 10 KCl, 110 KF, 10 EGTA, and 10 HEPES (pH adjusted to 7.2 with KOH, osmolarity 285-295 mOsm).

Test and positive control (dofetilide) compounds were solubilized in 100% DMSO at 10 mM or 75 mM, respectively. Intermediate dilutions of compounds were prepared by serial dilution in 100% DMSO, after which working solutions were prepared in ECS (final DMSO concentration=0.1-1% v/v for test compounds, and 0.1% for dofetilide). Final concentrations for IC₅₀ determination in hERG inhibition dose-response assays were 20 and 1 mM (test compounds) or 150, 50, 16.67, 5.56 and 1.85 nM (dofetilide).

Whole cell recordings of voltage-clamped cells were performed at 35° C., with all working solutions heated to 34° C. in a water bath before administration. Current recordings were collected at 20 kHz with a 10 kHz filter and were compensated for capacitance. Cells were clamped at −60 mV, and leak current was tested at −80 mV for 500 ms. The hERG current was elicited by depolarizing at +30 mV for 4.8 seconds and then the voltage was taken back to −50 mV for 5.2 seconds to remove the inactivation and observe the deactivating tail current. Recordings were continued for 120 seconds to assess current stability. Only stable cells with recording parameters above threshold were used in compound administration experiments. Vehicle control was applied to cells to establish baseline. Once the hERG current was found to be stabilized for 5 minutes, working compound solution was applied. hERG current in the presence of test compound was recorded for approximately 5 minutes to reach steady state and then 5 sweeps were captured. For dose response testing, test compound was applied to the cells cumulatively from low to high concentrations. Dofetilide at concentration of 450 nM was also applied to each cell post-hERG current measurement at the highest concentration of test compound as the internal low control for normalization of percentage inhibition. Dofetilide (5 doses) response was performed as positive hERG inhibition control for each batch of cells.

The following criteria were used to determine data acceptability: 1) Initial seal resistance>1 GΩ; 2) Leak currents<50% of the control peak tail currents at any time; 3) Normal test pulse current waveform (e.g., hERG peak tail) current amplitude greater than prepulse current amplitude and the peak tail amplitude>250 pA; 4) Membrane resistance Rm>500 MΩ; 5) Access resistance (Ra)<15 MΩ; 6) Apparent run-down of peak current<2.5% per min. Percent current inhibition was calculated as (1−(Peak tail current_{compound}−Peak tail current_{positive control})/(Peak tail current_{Blank vehicle}−Peak tail current_{positive control}))×100. PatchMaster software was used to extract the peak current from the original data. The dose response curve of test compounds was plotted with % inhibition against the concentration of test compounds using Graphpad Prism 8.0, and the data fit to a sigmoid dose-response curve with a variable slope.

TABLE 56

| | hERG Inhibition (%) | hERG Inhibition (%) |
|---|---|---|
| Compound # | @ 1 μM | @ 20 μM |
| 1 | 10 | 35 |
| 5 | 3 | 35 |
| 11 | 11 | 62 |
| 489 | 12 | 47 |
| 494 | 9 | 54 |

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula (I) or a pharmaceutically acceptable salt thereof: 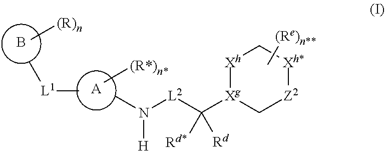
wherein the “*” at the carbon to which R^{d} and R^{d*} are bonded is a first stereo center when R^{d} and R^{d*} are different;
Z² is CH₂ or 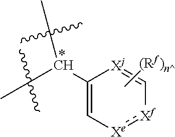
wherein the wavy lines represent the points of attachment of the Z² group to the X^{h*} and the —CH₂ of the 6-membered ring;
wherein the “*” at the carbon atom in Z² designates a second stereocenter;
with the proviso that when Z² is —CH₂, then Ring A is not a thiazolyl;
Ring B is a 6-membered aryl or 5- or 6-membered heteroaryl group;
each R is independently selected from hydrogen, CN, SF₅, halo, (C₁-C₆)alkyl, —(C₀-C₆)alkyl-NH₂, —(C₀-C₆)alkyl-NH((C₁-C₆)alkyl), —(C₀-C₆)alkyl-N((C₁-C₆)alkyl)((C₁-C₆)alkyl), —(C₀-C₆)alkyl-(C₃-C₈)cycloalkyl, —(C₀-C₆)alkyl-aryl, 5- or 6-membered heteroaryl(C₁-C₄)alkyl-, (C₂-C₆)alkenyl, —(C₂-C₆)alkenyl-(C₃-C₈)cycloalkyl, —OH, (C₁-C₄)alkoxy, —O—(C₁-C₆)alkyl-(C₃-C₈)cycloalkyl, —O—(C₁-C₆)alkyl-aryl, —O—(C₁-C₆)alkyl-5-6 membered heteroaryl, —O—(C₂-C₆)alkenyl, —O—(C₃-C₈)cycloalkyl, —O-aryl, —O-heteroaryl, —C(O)-aryl, —CO₂H, —CO₂(C₁-C₆)alkyl, —CO₂(C₃-C₈)cycloalkyl, —O₂C(C₁-C₆)alkyl, —O₂C(C₃-C₈)cycloalkyl, or 5-6 membered heteroaryl, wherein any said (C₁-C₆)alkyl, —(C₀-C₆)alkyl-(C₃-C₈)cycloalkyl, —(C₀-C₆)alkyl-aryl, (C₁-C₄)alkoxy, —O—(C₁-C₆)alkyl-(C₃-C₈)cycloalkyl, —O—(C₃-C₈)cycloalkyl, —O-aryl, 5-6 membered heteroaryl, —C(O)-aryl, or 5-6 membered heteroaryl is optionally substituted one, two, or three times by R¹ wherein each R¹ is independently halo, —CN, —SF₅, —OH, (C₁-C₆)alkyl, (C₁-C₄)alkoxy, (C₃-C₈)cycloalkyl, —(C₁-C₆)—OH, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), —O—(C₃-C₈)cycloalkyl, -aryl, 5-6 membered heteroaryl, wherein (C₁-C₆)alkyl, (C₃-C₈)cycloalkyl, aryl, or 5-6 membered heteroaryl is further optionally substituted by one, two, or three substituents independently selected from halo, (C₁-C₆)alkyl, —(C₁-C₆)alkyl-OH, —(C₁-C₆)alkyl-O—(C₁-C₆)alkyl, or (C₁-C₄)alkoxy;
n is 0 or an integer from 1 to 5;
L¹ is C(R^{c})(R^{c*}), O, SO₂ or C(O);
each of R^{c} and R^{c*} is independently H, D, halo, (C₁-C₆)alkyl, or (C₂-C₆)alkenyl; or R^{c} and R^{c*} taken together with the atom to which they are attached, form a 3-, 4-, 5-, 6-, or 7-membered ring optionally containing one, two, or three heteroatoms independently selected from oxygen, nitrogen, and sulfur; wherein said ring is optionally substituted by one or two substituents independently selected from halo, (C₁-C₆)alkyl, halo(C₁-C₆)alkyl, and (C₃-C₈)cycloalkyl;
Ring A is a 5-membered arylene or a 5-membered heteroarylene group;
each R* is independently selected from hydrogen, OH, halo, (C₁-C₆)alkyl, halo(C₁-C₆)alkyl, (C₂-C₆)alkenyl, —(C₁-C₆)alkyl-NH₂, —(C₁-C₆)alkyl-NH((C₁-C₆)alkyl), —(C₁-C₆)alkyl-N((C₁-C₆)alkyl)((C₁-C₆)alkyl), (C₂-C₆)alkenyl, —O—(C₁-C₆)alkyl, —NH₂, —NH((C₁-C₆)alkyl), —N((C₁-C₆)alkyl)((C₁-C₆)alkyl), —NCH₂, or —CHNH; or two R* groups taken together with the atoms to which they are attached, form a 5-, 6-, or 7-membered ring optionally containing one, two, or three heteroatoms independently selected from oxygen, nitrogen, and sulfur, wherein said ring is optionally substituted by one or two substituents independently selected from halo, (C₁-C₆)alkyl, —(C₁-C₆)alkyl-(C₃-C₈)cycloalkyl, halo(C₁-C₆)alkyl, and (C₃-C₈)cycloalkyl;
n* is 0 or an integer from 1 to 3;
or one of R^{c} or R^{c*} and R* taken together with the atoms to which they are bonded form a 3- to 6-membered ring fused to Ring A;
L² is —C(O)— or 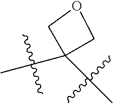 [SMILES: *C1(*)COC1]
wherein the wavy line represents the points of attachment to the NH and the C(R^{d})(R^{d*}) groups;
R^{d} and R^{d*} are each independently H or (C₁-C₃)alkyl, or (C₁-C₃)alkoxy, or R^{d} and R^{d*} cyclize to form a 3 to 6 membered cycloalkyl or heterocycloalkyl ring;
X^{h} and X^{h*} are each independently C(R^{e})₂, N(R^{e}), or O;
X^{g} is CH or N;
each R^{e} is independently H, halo, (C₁-C₃)alkyl, halo(C₁-C₃)alkyl, or (C₁-C₃)alkoxy, or any two R^{e} groups, on the same ring atom or on adjacent ring atoms, cyclize to form a 3 to 6 membered fused or spiro ring;
n** is 0 or an integer from 1 to 8;
X^{j} is N, NR^{f}, C(R^{f})₂, C(R^{f}) or CH;
X^{e} is N, NR^{f}, C(R^{f})₂ or C(R^{f});
X^{f} is SO₂, N, C-(halo(C₁-C₃)alkyl), C—(C₁-C₃)alkyl, C═O or N⁺—O—; [symbol]
represents a double or single bond as needed to satisfy atom valences;
each R^{f} is independently H or halo, (C₁-C₆)alkyl, (C₃-C₈)cycloalkyl, (C₁-C₄)alkoxy, —(C₁-C₆)alkyl-OH, —(C₁-C₆)alkyl-O—(C₁-C₆)alkyl, —(C₁-C₆)alkyl-NH₂, —(C₁-C₆)alkyl-NH((C₁-C₆)alkyl), —(C₁-C₆)alkyl-N—((C₁-C₆)alkyl)((C₁-C₆)alkyl), —(C₁-C₆)alkyl-NH((C₁-C₆)alkyl)-OH, —(C₁-C₆)alkyl-NH((C₁-C₆)alkyl)-O—(C₁-C₆)alkyl), —(C₁-C₆)alkyl-N—((C₁-C₆)alkyl)((C₁-C₆)alkyl)-OH, —(C₁-C₆)alkyl-N—((C₁-C₆)alkyl)((C₁-C₆)alkyl)-O—(C₁-C₆)alkyl), —(C₁-C₆)alkyl-NH—(C₃-C₈)cycloalkyl, —(C₁-C₆)alkyl-NH-4- to 6-membered heterocycloalkyl, —(C₁-C₆)alkyl-4- to 6-membered heterocycloalkyl, —(C₁-C₆)alkyl-NHC(O)—((C₁-C₆)alkyl), —(C₁-C₆)alkyl-SO₂—(C₁-C₆)alkyl, —(C₁-C₆)alkyl-SO₂—NH—(C₁-C₆)alkyl, —CN, —C(O)NH₂, —C(O)NH(C₁-C₆)alkyl), —C(O)N(C₁-C₆)alkyl) (C₁-C₆)alkyl), —NH₂, —NH(C₁-C₆)alkyl, —N((C₁-C₆)alkyl)((C₁-C₆)alkyl), —SO₂((C₁-C₆)alkyl), —SO₂—NH((C₁-C₆)alkyl), or aryl, wherein any said (C₁-C₆)alkyl, (C₃-C₈)cycloalkyl, —(C₁-C₆)alkyl-OH, —(C₁-C₆)alkyl-NH₂, —(C₁-C₆)alkyl-NH((C₁-C₆)alkyl), —(C₁-C₆)alkyl-NH—(C₃-C₈)cycloalkyl, —(C₁-C₆)alkyl-NH-4- to 6-membered heterocycloalkyl, or —(C₁-C₆)alkyl-4- to 6-membered heterocycloalkyl is optionally substituted one, two, or three times by halo, OH, or any two R^{f} groups, on the same ring atom or on adjacent ring atoms, cyclize to form a 3 to 6 membered fused or spiro ring;
n\hat{ } is 0 or an integer from 1 to 4.

2. (canceled)

3. The compound of claim 1, wherein X^{g} is N.

4.-6. (canceled)

7. The compound of claim 1, wherein each R^{d} and R^{d*} are independently chosen from H and CH₃.

8. The compound of claim 1, wherein R^{d} and R^{d*} are different, Z² is 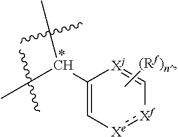
and the first and second stereocenters are both S.

9. The compound of claim 1, wherein R^{d} and R^{d*} are different, Z² is 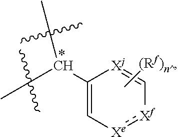
and wherein the first and second stereocenters are both R, or wherein the first stereocenter is S and second stereocenter is R, or wherein the first stereocenter is R and second stereocenter is S.

10. (canceled)

11. The compound of claim 1, wherein at least one R^{e} is halo, (C₁-C₃)alkyl, or (C₁-C₃)alkoxy.

12. The compound of claim 1, wherein each R^{e} is independently chosen from H, F, CH₃ and OCH₃.

13.-14. (canceled)

15. The compound of claim 1, wherein X^{h} is CH₂.

16. (canceled)

17. The compound of claim 1, wherein X^{h*} is O; or is C(R^{e})₂ and each R^{e} is F or two R^{e} groups bonded to a single carbon atom cyclize to form a 3 membered ring.

18. The compound of claim 1, wherein L² is C═O.

19. (canceled)

20. The compound of claim 1, wherein n* is 1.

21. (canceled)

22. The compound of claim 1, wherein Ring B is phenyl, pyridinyl, pyridazinyl, or pyrimidinyl.

23. (canceled)

24. The compound of claim 1, wherein each R is independently CN, SF₅, F, Cl, CH₃, OCH₃, O-phenyl, CF₃ or OCF₃.

25. (canceled)

26. The compound of claim 1, wherein n is 1, 2 or 3.

27. (canceled)

28. (canceled)

29. The compound of claim 1, wherein n\hat{ } is 0.

30. (canceled)

31. The compound of claim 1, wherein, L¹ is C(R^{c})(R^{c*}).

32. (canceled)

33. The compound of claim 31, wherein one of R^{c} or R^{c*} and R* taken together with the atoms to which they are bonded form an optionally substituted 3- to 6-membered ring fused to Ring A.

34. (canceled)

35. The compound of claim 31, wherein Ring A is imidazolylene, triazolylene, pyrazolylene, or thiazolylene; n* is 1 or 2; L¹ is C(R^{c})(R^{c*}) and one of R^{c} or R^{c*} and R* taken together with the atoms to which they are bonded form an optionally substituted 3- to 6-membered ring fused to Ring A.

36. (canceled)

37. The compound of claim 35, wherein the optionally substituted 3- to 6-membered ring fused to Ring A has the structure 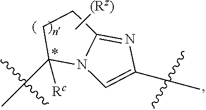
wherein R^{z} is absent or is OH, n′ is 0, 1 or 2, and the “*” at the ring carbon bonded to Ring B represents a third stereocenter.

38. The compound of claim 37, wherein n′ is 1.

39. The compound of claim 37, wherein R^{c} is H.

40. The compound of claim 35, wherein the 3- to 6-membered ring fused to Ring A has a structure selected from: 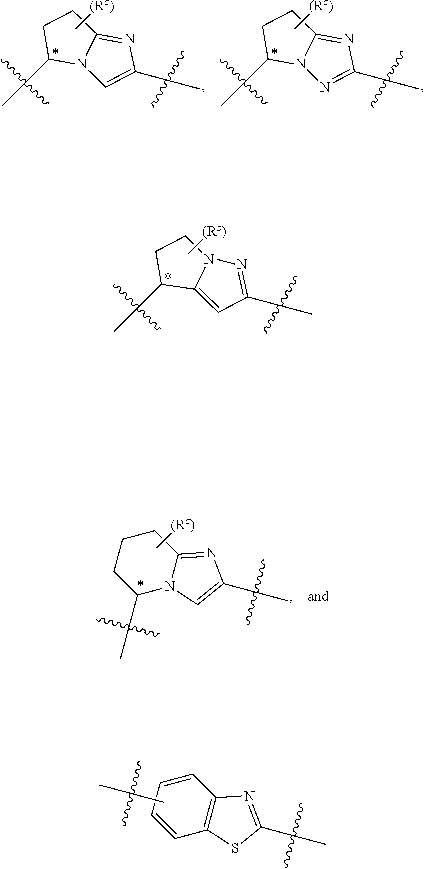
wherein R^{z} is absent or is OH, and the “*” at the ring carbon bonded to Ring B represents a third stereocenter.

41. (canceled)

42. The compound of claim 35, wherein the 3- to 6-membered ring fused to Ring A forms 6,7-dihydro-5H-pyrrolo[1,2-a]imidazol-2-ylene.

43.-44. (canceled)

45. The compound of any one of claims 1 to 42, wherein X^{e} is CH and X^{f} is N⁺—O⁻ and the bond between X^{e} and X^{f} is a double bond.

46.-81. (canceled)

82. The compound of claim 1 or a pharmaceutically acceptable salt thereof, wherein the compound of Formula (I) is Formula (Ie): 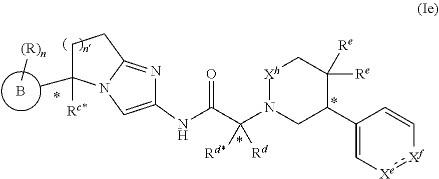
wherein n′ is 0, 1 or 2 and the “*” at the carbon bonded to both Ring B and R^{c*} represents a third stereocenter, R^{d} is H and R^{d*} is Me, Ring B is phenyl or pyridinyl, n is 1, 2 or 3, and each R^{e} is independently halo, (C₁-C₃)alkyl, or (C₁-C₃)alkoxy.

83.-86. (canceled)

87. The compound of claim 82, wherein each R is independently CN, SF₅, F, methyl, OCH₃, O-phenyl, or CF₃.

88.-90. (canceled)

91. The compound of claim 82, wherein each R^{e} is independently F, Me, or MeO.

92.-95. (canceled)

96. The compound of claim 82, wherein X^{e} is CH and X^{f} is N⁺—O⁻ and the bond between X^{e} and X^{f} is a double bond.

97. (canceled)

98. The compound of claim 1 having a structure chosen from: 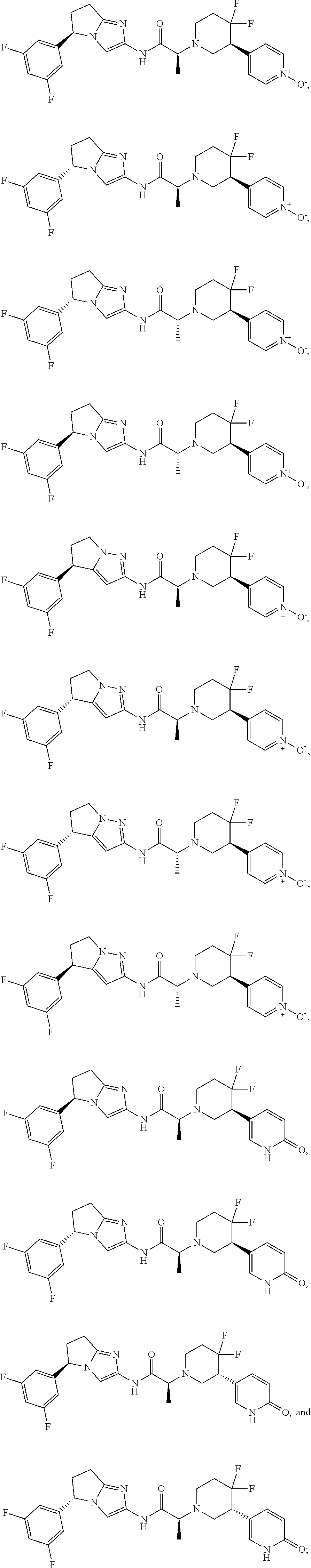
or a pharmaceutically acceptable salt thereof.

99. A compound having the structure: 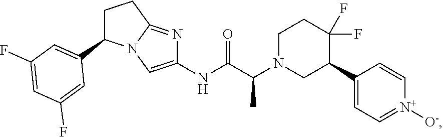
or a pharmaceutically acceptable salt thereof.

100. A compound having the structure 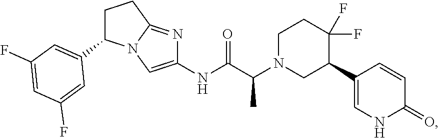 [SMILES: C[C@@H](C(=O)Nc1cn2c(n1)CC[C@H]2c1cc(F)cc(F)c1)N1CCC(F)(F)[C@@H](c2ccc(=O)[nH]c2)C1]
or a pharmaceutically acceptable salt thereof.

101. (canceled)

102. A compound chosen from
2-(4,4-difluoro-3-(6-oxo-1,6-dihydropyridin-3-yl)piperidin-1-yl)-N-(5-(3,5-difluorophenyl)-6,7-dihydro-5H-pyrrolo[1,2-a]imidazol-2-yl)propanamide;
(S)-2-((S)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide;
(S)-2-((S)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide;
(S)-2-((R)-4,4-difluoro-3-(6-oxo-1,6-dihydropyridin-3-yl)piperidin-1-yl)-N—((R)-5-(3,5-difluorophenyl)-6,7-dihydro-5H-pyrrolo[1,2-a]imidazol-2-yl)propanamide;
(S)-2-((R)-4,4-difluoro-3-(6-oxo-1,6-dihydropyridin-3-yl)piperidin-1-yl)-N—((S)-5-(3,5-difluorophenyl)-6,7-dihydro-5H-pyrrolo[1,2-a]imidazol-2-yl)propanamide;
4-(1-(1-((5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((S)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-((S)-1-((R)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide; and
4-((S)-1-((R)-1-(((S)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide;
4-(1-(1-((4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((S)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((S)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((R)-1-(((S)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
4-((S)-1-((R)-1-(((R)-4-(3,5-difluorophenyl)-5,6-dihydro-4H-pyrrolo[1,2-b]pyrazol-2-yl)amino)-1-oxopropan-2-yl)-4,4-difluoropiperidin-3-yl)pyridine 1-oxide;
or a pharmaceutically acceptable salt thereof.

103. A compound 4-((S)-1-((S)-1-(((R)-5-(3,5-difluorophenyl)-6,7-dihydro-5H-pyrrolo[1,2-a]imidazol-2-yl)amino)-1-oxopropan-2-yl)-4,4-difluoropiperidin-3-yl)pyridine 1-oxide, or a pharmaceutically acceptable salt thereof.

104.-105. (canceled)

106. A pharmaceutical composition comprising a compound and/or a pharmaceutically acceptable salt of claim 1 and a pharmaceutically acceptable excipient.

107. A method of modulating MRGPRX2 activity, the method comprising administering an effective amount of a compound of claim 1.

108. A method of modulating mast cell degranulation comprising administering an effective amount of a compound of claim 1.

109. A method of treating, preventing or ameliorating an MRGPRX2-mediated disease or disorder in a subject in need thereof comprising administering to the subject an effective amount of a compound of claim 1.

110. The method of claim 109, wherein the disease is chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain.

111. A method of treating a condition chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain comprising administering to the subject an effective amount of a compound of claim 1.

112. The method of claim 111, wherein the condition is atopic dermatitis.
